(19) United States
(12) Patent Application Publication
(10) Pub. No.: US 2024/0199571 A1
(43) Pub. Date: Jun. 20, 2024

(54) THIOPHENE COMPOUND AND APPLICATION THEREOF

(71) Applicant: MEDSHINE DISCOVERY INC., Jiangsu (CN)

(72) Inventors: Lingyun WU, Shanghai (CN); Zhen ZHAN, Shanghai (CN); Lele ZHAO, Shanghai (CN); Jianjun SUN, Shanghai (CN); Yi QIAN, Shanghai (CN); Shuhui CHEN, Shanghai (CN)

(21) Appl. No.: 18/549,188

(22) PCT Filed: Mar. 4, 2022
(86) PCT No.: PCT/CN2022/079277

§ 371 Date: Sep. 6, 2023

(30) Foreign Application Priority Data
Mar. 11, 2021 (CN) 202110268287.6
May 25, 2021 (CN) 202110573989.5
Oct. 19, 2021 (CN) 202111217911.6
Feb. 24, 2022 (CN) 202210172993.5

(51) Int. Cl. C07D 333/22; A61K 31/381; A61K 31/407; A61K 31/438; A61K 31/439; A61K 31/495; A61K 31/551; C07D 403/04; C07D 413/04

(52) U.S. Cl. CPC C07D 333/22; A61K 31/381; A61K 31/407; A61K 31/438; A61K 31/439; A61K 31/495; A61K 31/551; C07D 403/04; C07D 413/04

(57) ABSTRACT

The present invention relates to a thiophene compound and an application thereof. The present invention specifically relates to a compound represented by formula (II) and a pharmaceutically acceptable salt thereof, wherein L₁ is —C(═O)— or —C(═O)—NH—; R₁ is R₁ₐ or aa; ring A is bb or cc; R₁ₐ is C₁₋₃ alkyl substituted with 1, 2 or 3 Rₐ; and R₃ is independently H, halogen or C₁₋₃ alkoxy. The compound represented by formula (II) and the pharmaceutically acceptable salt thereof can better inhibit the activity of LSD1, further shows significant inhibitory activity against NCI-H1417 cell proliferation, and has good pharmacokinetic properties (including good oral bioavailability, oral exposure, half-life, and clearance rate, etc.).

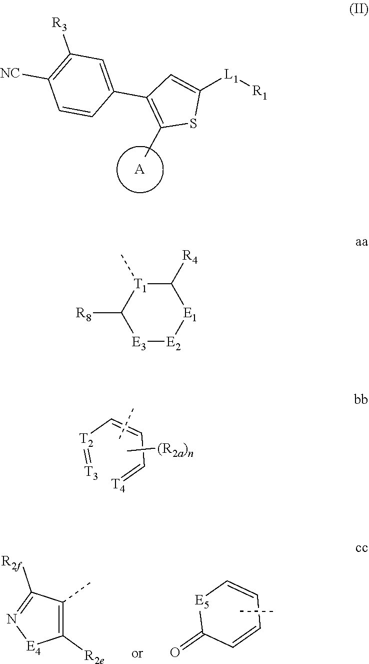

The present application claims the right of the following priorities:
CN202110268287.6, Mar. 11, 2021;
CN202110573989.5, May 25, 2021;
CN202111217911.6, Oct. 19, 2021;
CN202210172993.5, Feb. 24, 2022.

### TECHNICAL FIELD

The present disclosure relates to a class of thiophene compounds and a use thereof. The present disclosure specifically relates to a compound of formula (I) and a pharmaceutically acceptable salt thereof.

### BACKGROUND

Post-translational modifications of histones, which comprise processes such as methylation, acetylation, phosphorylation, and ubiquitination, are important regulatory mechanisms in epigenetics, affecting gene expression by altering chromatin structure. [Xueshun Wang, Boshi Huang, Takayoshi Suzuki et al. Epigenomics, 2015, 1379-1396]. Although these modifications do not alter the basic DNA sequence, such epigenetic alteration may persist through cell division throughout the cell life cycle or iteration process [Adrian Bird, Nature, 2007, 396-398]. Therefore, abnormalities in epigenetic function are closely associated with the pathological processes of various diseases [James T Lynch, William J Harris & Tim C P Somervaille, Expert Opin. Ther. Targets, 2012, 1239-1249], such as a variety of solid tumors, hematological tumors, viral infections, and neurological abnormalities. As a result, epigenetics has now become a research hotspot in the field of drug research and development. The methylation state of histones is jointly regulated by histone methyltransferases and histone demethylases. Lysine specific demethylase 1 (LSD1, also known as KDM1A) is the first reported histone lysine demethylase, which is widely involved in transcriptional regulation and affects many physiological processes such as cell proliferation and differentiation and embryonic stem cell pluripotency, by regulating the methylation state of histone lysine. [Yujiang Shi, Fei Lan, Caitlin Matson et al., Cell, 2004, 941-953] [Daniel P. Mould, Alison E. McGonagle, Daniel H. Wiseman et al., Medicinal Research Reviews, 2015, 586-618]. The LSD1 structure comprises three main parts: the N-terminal SWIRM domain, the C-terminal amine oxidase-like (AOL) domain, and the central Tower domain [Ruchi Anand, Ronen Marmorstein, Journal of Biological Chemistry, 2007, 35425-35429]. The C-terminal amine oxidase-like domain comprises two active pockets, one is the FAD binding site and the other is a site used to recognize and bind to substrates [Pete Stavropoulos, Ginter Blobel, Andre Hoelz, Nature Structural & Molecular Biology, 2006, 626-632]. The function of the SWIRM domain is not yet clear. It does not directly participate in FAD or substrate binding, but mutations or deletions in this domain will reduce LSD1 activity. Therefore, it is speculated that this domain may affect the active region by adjusting the conformation [Yong Chen, Yuting Yang, Feng Wang et al., Proceedings of the National Academy of Sciences, 2006, 13956-13961]. The Tower domain is the binding domain of LSD1 with other protein factors. LSD1 binds to different protein factors and acts on different substrates, thereby exerting different regulatory effects on histones as well as gene expression. For example, when LSD1 binds to CoREST, it acts preferentially on histone H3K4, removing activation-related histone marks and repressing gene transcription through demethylation; whereas, when bound to androgen receptor proteins, recombinant LSD1 acts preferentially on H3K9, activating androgen receptor-related gene transcription through demethylation [Ruchi Anand, Ronen Marmorstein, Journal of Biological Chemistry, 2007, 35425-35429; Eric Metzger, Melanie Wissmann, Na Yin et al. Nature, 2005, 436-439]. In addition, LSD1 also regulates the methylation state of some non-histone substrates, including oncogene p53, DNA methyltransferase 1 (DNMT1), etc. [Yi Chao Zheng, Jinlian Ma, Zhiru Wang, Medicinal Research Reviews, 2015, 1032-1071].

LSD1 is a FAD-dependent amine oxidase in which proton transfer is considered as its most likely oxidation mechanism [Zheng Y C, Yu B, Chen Z S, et al. Epigenomics, 2016, 8, 651-666]. First, the N—CH3 bond of the substrate is converted to an imine bond by proton transfer, and this imine ion intermediate undergoes a hydrolysis reaction to produce a demethylated amine on one side and formaldehyde on the other. During this catalytic cycle process, FAD is reduced to FADH2, which is subsequently oxidized back to FAD by a molecule of oxygen while generating a molecule of H₂O₂ [Yujiang Shi, Fei Lan, Caitlin Matson, Cell, 2004, 941-953].

LSD1 is abnormally expressed in various types of tumors. LSD1 is highly expressed in acute myeloid leukemia (AML) subtypes and is an important factor in maintaining the potential of leukemia stem cell (LSC). LSD1 is highly expressed in a variety of solid tumors such as lung cancer, breast cancer, prostate cancer, liver cancer, and pancreatic cancer, and is closely associated with poor tumor prognosis. LSD1 inhibits cadherin expression and is closely associated with tumor invasion and epithelial-mesenchymal transition (EMT). [Hosseini A, Minucci S. Epigenomics, 2017, 9, 1123-1142].

Currently, no drugs for LSD1 inhibitors are approved in the market. There are 8 drugs in clinical research, mainly used for the treatment of diseases such as hematological tumors, small cell lung cancer, and Ewing's sarcoma. However, in the face of a huge unmet market, candidate compounds with better activity and pharmacokinetic parameters are still needed in the field for advancing clinical trials to meet treatment needs.

### CONTENT OF THE PRESENT INVENTION

The present disclosure provides a compound of formula (II) or a pharmaceutically acceptable salt thereof,

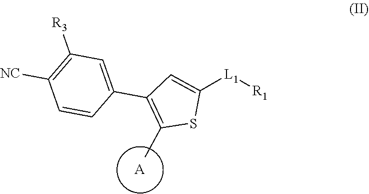

wherein L₁ is —C(═O)— or —C(═O)—NH—;
R₁ is R₁ₐ or

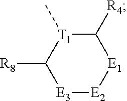

ring A is

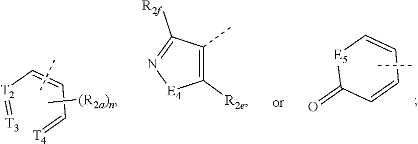

T₁ is CH or N;
T₂ is CR_{2c} or N;
T₃ is CR₂ or N;
T₄ is CR_{2b} or N;
E₁ is a single bond, —C(R₅)₂—, or —C(R₅)₂C(R₅)₂—;
E₂ is —NR₆— or —C(R₆₁)₂—;
E₃ is a single bond, —C(R₇)₂—, or —C(R₇)₂C(R₇)₂—;
E₄ is O, S, CH₂, or NR_{2g};
E₅ is O, CH₂, or NR_{2g};
R₁ₐ is C₁₋₃ alkyl substituted by 1, 2, or 3 Rₐ;
R₂ₐ is H or halogen;
R₂ and R_{2b} are each independently H, halogen, NH₂, C₁₋₃ alkyl, C₁₋₃ alkoxy, —S(═O)₂C₁₋₃ alkyl, —NHC₁₋₃ alkyl, 4- to 6-membered heterocycloalkyl, —C(═O)-4- to 6-membered heterocycloalkyl, or —NHC₁₋₃ alkyl-C₃₋₆ cycloalkyl, wherein the C₁₋₃ alkyl, C₁₋₃ alkoxy, —S(═O)₂C₁₋₃ alkyl, —NHC₁₋₃ alkyl, 4- to 6-membered heterocycloalkyl, —C(═O)-4- to 6-membered heterocycloalkyl, and —NHC₁₋₃ alkyl-C₃₋₆ cycloalkyl are each independently and optionally substituted by 1, 2, or 3 R_{b};
R_{2c} is H;
or, R₂ and R_{2c}, together with the C atom to which they are attached, make the structural moiety

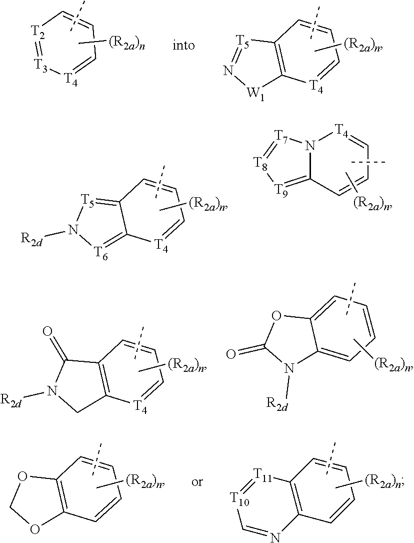

T₅, T₆, T₇, T₈, T₉, T₁₀, and T₁₁ are each independently N or CRₜ;
W₁ is O or NR_{2d};
R_{2d} is H, C₁₋₄ alkyl, C₃₋₆ cycloalkyl, 4- to 6-membered heterocycloalkyl, —C₁₋₃ alkyl-C₃₋₆ cycloalkyl, or —C₁₋₃ alkyl-4- to 6-membered heterocycloalkyl, wherein the C₁₋₄ alkyl, C₃₋₆ cycloalkyl, 4- to 6-membered heterocycloalkyl, —C₁₋₃ alkyl-C₃₋₆ cycloalkyl, and —C₁₋₃ alkyl-4- to 6-membered heterocycloalkyl are each independently and optionally substituted by 1, 2, or 3 R_{c};
R₂ₑ, R_{2f}, and R_{2g} are each independently H or C₁₋₃ alkyl;
each R₃ is independently H, halogen, or C₁₋₃ alkoxy;
R₄ and R₈ are each independently H or C₁₋₃ alkyl;
each R₅ is independently H, halogen, OH, NH₂, C₁₋₃ alkyl, —NHC₁₋₃ alkyl, or —N(C₁₋₃ alkyl)₂, wherein the C₁₋₃ alkyl, —NHC₁₋₃ alkyl, and —N(C₁₋₃ alkyl)₂ are each independently and optionally substituted by 1, 2, or 3 R_{d};
R₆ is H or C₁₋₃ alkyl, wherein the C₁₋₃ alkyl is optionally substituted by 1, 2, or 3 Rₑ;
each R₆₁ is independently H, halogen, OH, NH₂, or C₁₋₃ alkyl, wherein the C₁₋₃ alkyl is optionally substituted by 1, 2, or 3 R_{f};
each R₇ is independently H, halogen, OH, NH₂, C₁₋₃ alkyl, —NHC₁₋₃ alkyl, or —N(C₁₋₃ alkyl)₂, wherein the C₁₋₃ alkyl, —NHC₁₋₃ alkyl, and —N(C₁₋₃ alkyl)₂ are each independently and optionally substituted by 1, 2, or 3 R_{g};
or, when E₁ is —C(R₅)₂—, E₂ is —C(R₆₁)₂—, and E₃ is a single bond, R₅ and R₆₁, together with the C atom to which they are attached, make the structural moiety

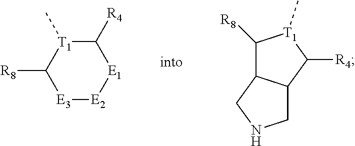

or, when E₂ is —C(R₆₁)₂—, two R₆₁, together with the C atom to which they are attached, make the structural moiety

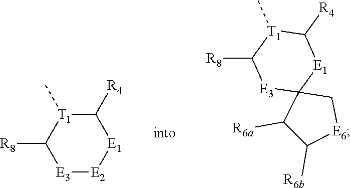

or, when E₃ is a single bond, R₄ and R₈, together with the C atom to which they are attached, make the structural moiety

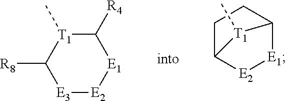

or, when T₁ is CH and E₁ is —C(R₅)₂—, R₅ and R₈, together with the C atom to which they are attached, make the structural moiety

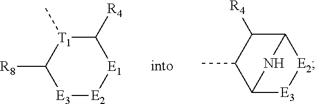

E₆ is NR_{6c} or O;
R₆ₐ and R_{6b} are each independently H, halogen, OH, ═O, NH₂, or C₁₋₃ alkyl;
R_{6c} is H or C₁₋₃ alkyl;
Rₐ, R_{b}, R_{c}, R_{d}, Rₑ, R_{f}, and R_{g} are each independently F, Cl, Br, OH, CN, COOH, NH₂, C₁₋₃ alkyl, or C₁₋₃ alkoxy;
each Rₜ is independently H, F, Cl, Br, OH, CN, COOH, NH₂, C₁₋₃ alkyl, or C₁₋₃ alkoxy;
n is 1 or 2;
the 4- to 6-membered heterocycloalkyl contains 1, 2, 3, or 4 heteroatoms or heteroatom groups independently selected from —O—, —NH—, —S—, and N.

The present disclosure provides a compound of formula (II) or a pharmaceutically acceptable salt thereof,

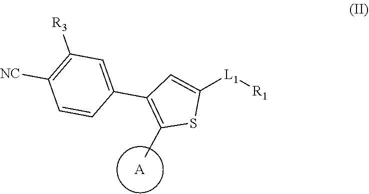

wherein L₁ is —C(═O)— or —C(═O)—NH—;
R₁ is R₁ₐ or

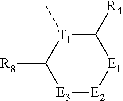

ring A is

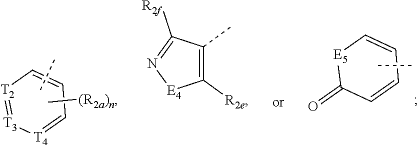

T₁ is CH or N;
T₂ is CR_{2c} or N;
T₃ is CR₂ or N;
T₄ is CR_{2b} or N;
E₁ is a single bond, —C(R₅)₂—, or —C(R₅)₂C(R₅)₂—;
E₂ is —NR₆— or —C(R₆₁)₂—;
E₃ is a single bond, —C(R₇)₂—, or —C(R₇)₂C(R₇)₂—;
E₄ is O, S, CH₂, or NR_{2g};
E₅ is O, CH₂, or NR_{2g};
R₁ₐ is C₁₋₃ alkyl substituted by 1, 2, or 3 Rₐ;
R₂ₐ is H or halogen;
R₂ and R_{2b} are each independently H, halogen, NH₂, C₁₋₃ alkyl, C₁₋₃ alkoxy, —S(═O)₂C₁₋₃ alkyl, —NHC₁₋₃ alkyl, 4- to 6-membered heterocycloalkyl, —C(═O)-4- to 6-membered heterocycloalkyl, or —NHC₁₋₃ alkyl-C₃₋₆ cycloalkyl, wherein the C₁₋₃ alkyl, C₁₋₃ alkoxy, —S(═O)₂C₁₋₃ alkyl, —NHC₁₋₃ alkyl, 4- to 6-membered heterocycloalkyl, —C(═O)-4- to 6-membered heterocycloalkyl, and —NHC₁₋₃ alkyl-C₃₋₆ cycloalkyl are each independently and optionally substituted by 1, 2, or 3 R_{b};
R_{2c} is H;
or, R₂ and R_{2c}, together with the C atom to which they are attached, make the structural moiety

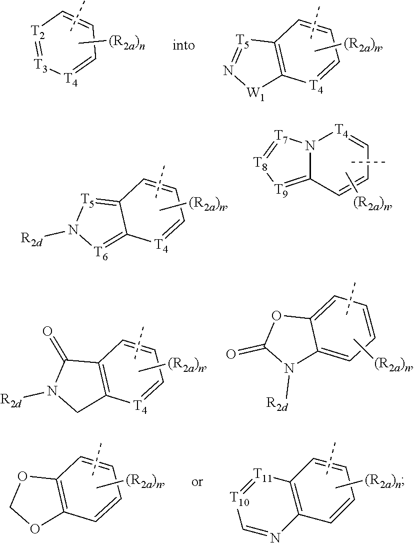

T₅, T₆, T₇, T₈, T₉, T₁₀, and T₁₁ are each independently N or CRₜ;
W₁ is O or NR_{2d};
R_{2d} is H, C₁₋₄ alkyl, C₃₋₆ cycloalkyl, 4- to 6-membered heterocycloalkyl, —C₁₋₃ alkyl-C₃₋₆ cycloalkyl, or —C₁₋₃ alkyl-4- to 6-membered heterocycloalkyl, wherein the C₁₋₄ alkyl, C₃₋₆ cycloalkyl, 4- to 6-membered heterocycloalkyl, —C₁₋₃ alkyl-C₃₋₆ cycloalkyl, and —C₁₋₃ alkyl-4- to 6-membered heterocycloalkyl are each independently and optionally substituted by 1, 2, or 3 R_{c};
R₂ₑ, R_{2f}, and R_{2g} are each independently H or C₁₋₃ alkyl;
each R₃ is independently H, halogen, or C₁₋₃ alkoxy;
R₄ and R₈ are each independently H or C₁₋₃ alkyl;
each R₅ is independently H, halogen, OH, NH₂, C₁₋₃ alkyl, —NHC₁₋₃ alkyl, or —N(C₁₋₃ alkyl)₂, wherein the C₁₋₃ alkyl, —NHC₁₋₃ alkyl, and —N(C₁₋₃ alkyl)₂ are each independently and optionally substituted by 1, 2, or 3 R_{d};
R₆ is H or C₁₋₃ alkyl, wherein the C₁₋₃ alkyl is optionally substituted by 1, 2, or 3 Rₑ;
each R₆₁ is independently H, halogen, OH, NH₂, or C₁₋₃ alkyl, wherein the C₁₋₃ alkyl is optionally substituted by 1, 2, or 3 R_{f};
each R₇ is independently H, halogen, OH, NH₂, C₁₋₃ alkyl, —NHC₁₋₃ alkyl, or —N(C₁₋₃ alkyl)₂, wherein the C₁₋₃ alkyl, —NHC₁₋₃ alkyl, and —N(C₁₋₃ alkyl)₂ are each independently and optionally substituted by 1, 2, or 3 R_{g};
or, when E₁ is —C(R₅)₂—, E₂ is —C(R₆₁)₂—, and E₃ is a single bond, R₅ and R₆₁, together
with the C atom to which they are attached, make the structural moiety

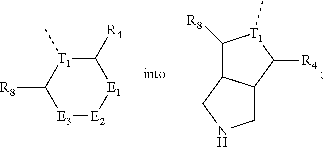

or, when E₂ is —C(R₆₁)₂—, two R₆₁, together with the C atom to which they are attached, make the structural moiety

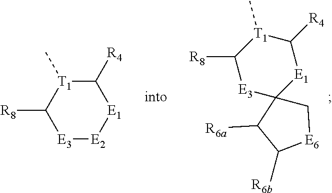

or, when E₃ is a single bond, R₄ and R₈, together with the C atom to which they are attached, make the structural moiety

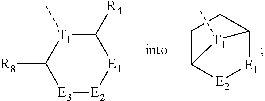

or, when T₁ is CH and E₁ is —C(R₅)₂—, R₅ and R₈, together with the C atom to which they are attached, make the structural moiety

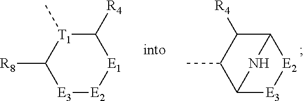

E₆ is NR_{6c} or O;
R₆ₐ and R_{6b} are each independently H, halogen, OH, ═O, NH₂, or C₁₋₃ alkyl;
R_{6c} is H or C₁₋₃ alkyl;
Rₐ, R_{b}, R_{c}, R_{d}, Rₑ, R_{f}, R_{g}, and Rₜ are each independently F, Cl, Br, OH, CN, COOH, NH₂, C₁₋₃ alkyl, or C₁₋₃ alkoxy;
n is 1 or 2;
the 4- to 6-membered heterocycloalkyl contains 1, 2, 3, or 4 heteroatoms or heteroatom groups independently selected from —O—, —NH—, —S—, and N.

The present disclosure provides a compound of formula (II) or a pharmaceutically acceptable salt thereof,

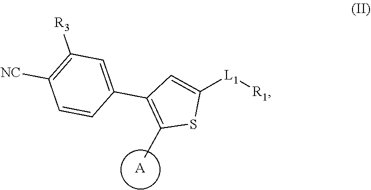

wherein L₁ is —C(═O)— or —C(═O)—NH—;
R₁ is R₁ₐ or

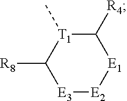

ring A is

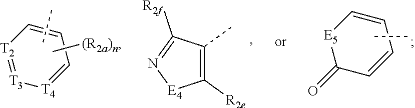

T₁ is CH or N;
T₂ is CR_{2c} or N;
T₃ is CR₂ or N;
T₄ is CR_{2b} or N;
E₁ is a single bond, —C(R₅)₂—, or —C(R₅)₂C(R₅)₂—;
E₂ is —NR₆— or —C(R₆₁)₂—;
E₃ is a single bond, —C(R₇)₂—, or —C(R₇)₂C(R₇)₂—;
E₄ is O, S, CH₂, or NR_{2g};
E₅ is O, CH₂, or NR_{2g};
R₁ₐ is C₁₋₃ alkyl substituted by 1, 2, or 3 Rₐ;
R₂ₐ is H or halogen;
R₂ and R_{2b} are each independently H, halogen, NH₂, C₁₋₃ alkyl, C₁₋₃ alkoxy, —S(═O)₂C₁₋₃ alkyl, 4- to 6-membered heterocycloalkyl, or —C(═O)-4- to 6-membered heterocycloalkyl, wherein the C₁₋₃ alkyl, C₁₋₃ alkoxy, —S(═O)₂C₁₋₃ alkyl, 4- to 6-membered heterocycloalkyl, and —C(═O)-4- to 6-membered heterocycloalkyl are each independently and optionally substituted by 1, 2, or 3 R_{b};
R_{2c} is H;
or, R₂ and R_{2c}, together with the C atom to which they are attached, make the structural moiety

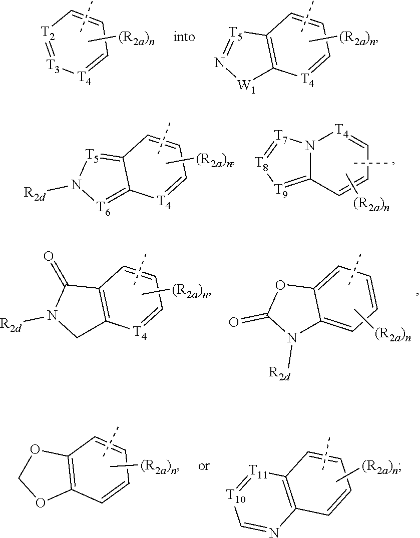

T₅, T₆, T₇, T₈, T₉, T₁₀, and T₁₁ are each independently N or CRₜ;
W₁ is O or NR_{2d};
R_{2d} is H, C₁₋₄ alkyl, C₃₋₆ cycloalkyl, 4- to 6-membered heterocycloalkyl, —C₁₋₃ alkyl-C₃₋₆ cycloalkyl, or —C₁₋₃ alkyl-4- to 6-membered heterocycloalkyl, wherein the C₁₋₄ alkyl, C₃₋₆ cycloalkyl, 4- to 6-membered heterocycloalkyl, —C₁₋₃ alkyl-C₃₋₆ cycloalkyl, and —C₁₋₃ alkyl-4- to 6-membered heterocycloalkyl are optionally substituted by 1, 2, or 3 R_{c};
R₂ₑ, R_{2f}, and R_{2g} are each independently H or C₁₋₃ alkyl;
each R₃ is independently H, halogen, or C₁₋₃ alkoxy;
R₄ and R₈ are each independently H or C₁₋₃ alkyl;
each R₅ is independently H, halogen, OH, NH₂, C₁₋₃ alkyl, —NHC₁₋₃ alkyl, or —N(C₁₋₃ alkyl)₂, wherein the C₁₋₃ alkyl, —NHC₁₋₃ alkyl, and —N(C₁₋₃ alkyl)₂ are each independently and optionally substituted by 1, 2, or 3 R_{d};
R₆ is H or C₁₋₃ alkyl, wherein the C₁₋₃ alkyl is optionally substituted by 1, 2, or 3 Rₑ;
each R₆₁ is independently H, halogen, OH, NH₂, or C₁₋₃ alkyl, wherein the C₁₋₃ alkyl is optionally substituted by 1, 2, or 3 R_{f};
each R₇ is independently H, halogen, OH, NH₂, C₁₋₃ alkyl, —NHC₁₋₃ alkyl, or —N(C₁₋₃ alkyl)₂, wherein the C₁₋₃ alkyl, —NHC₁₋₃ alkyl, and —N(C₁₋₃ alkyl)₂ are each independently and optionally substituted by 1, 2, or 3 R_{g};
or, when E₁ is —C(R₅)₂—, E₂ is —C(R₆₁)₂—, and E₃ is a single bond, R₅ and R₆₁, together with the C atom to which they are attached, make the structural moiety

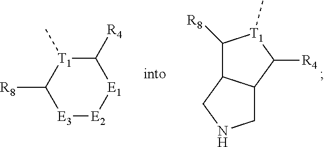

or, when E₂ is —C(R₆₁)₂—, two R₆₁, together with the C atom to which they are attached, make the structural moiety

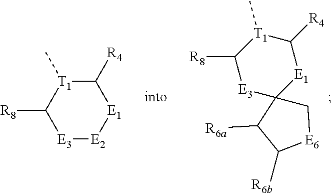

or, when E₃ is a single bond, R₄ and R₈, together with the C atom to which they are attached, make the structural moiety

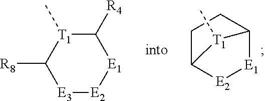

or, when T₁ is CH and E₁ is —C(R₅)₂—, R₅ and R₈, together with the C atom to which they are attached, make the structural moiety

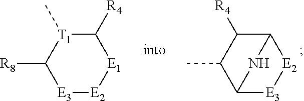

E₆ is NR_{6c} or O;
R₆ₐ and R_{6b} are each independently H, halogen, OH, ═O, NH₂, or C₁₋₃ alkyl;
R_{6c} is H or C₁₋₃ alkyl;
Rₐ, R_{b}, R_{c}, R_{d}, Rₑ, R_{f}, R_{g}, and Rₜ are each independently F, Cl, Br, OH, CN, COOH, NH₂, C₁₋₃ alkyl, or C₁₋₃ alkoxy;
n is 1 or 2;
the 4- to 6-membered heterocycloalkyl contains 1, 2, 3, or 4 heteroatoms or heteroatom groups independently selected from —O—, —NH—, —S—, and N.

The present disclosure provides a compound of formula (II) or a pharmaceutically acceptable salt thereof,

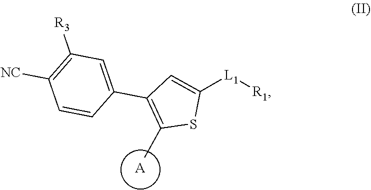

wherein L₁ is —C(═O)— or —C(═O)—NH—;
R₁ is R₁ₐ or

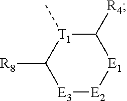

ring A is

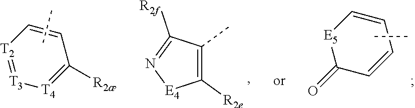

T₁ is CH or N;
T₂ is CR_{2c} or N;
T₃ is CR₂ or N;
T₄ is CR_{2b} or N;
E₁ is a single bond, —C(R₅)₂—, or —C(R₅)₂C(R₅)₂—;
E₂ is —NR₆— or —C(R₆₁)₂—;
E₃ is a single bond, —C(R₇)₂—, or —C(R₇)₂C(R₇)₂—;
E₄ is O, S, CH₂, or NR_{2g};
E₅ is O, CH₂, or NR_{2g};
R₁ₐ is C₁₋₃ alkyl substituted by 1, 2, or 3 Rₐ;
R₂ₐ is H or halogen;
R₂ and R_{2b} are each independently H, halogen, NH₂, C₁₋₃ alkyl, C₁₋₃ alkoxy, or —S(═O)₂C₁₋₃ alkyl, wherein the C₁₋₃ alkyl, C₁₋₃ alkoxy, and —S(═O)₂C₁₋₃ alkyl are each independently and optionally substituted by 1, 2, or 3 R_{b};
R_{2c} is H;
or, R₂ and R_{2c}, together with the C atom to which they are attached, make the structural moiety

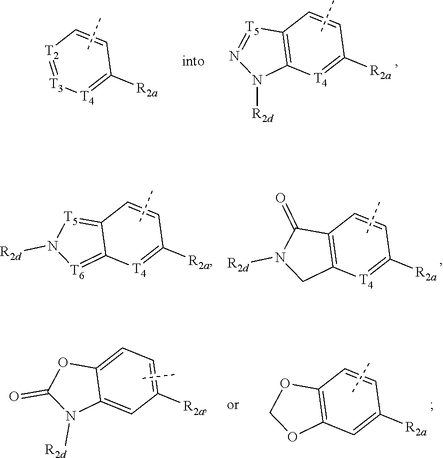

R_{2d} is H or C₁₋₄ alkyl, wherein the C₁₋₄ alkyl is optionally substituted by 1, 2, or 3 Rₑ;
R₂ₑ, R_{2f}, and R_{2g} are each independently H or C₁₋₃ alkyl;
T₅ and T₆ are each independently N or CH;
each R₃ is independently H, halogen, or C₁₋₃ alkoxy;
R₄ and R₈ are each independently H or C₁₋₃ alkyl;
each R₅ is independently H, halogen, OH, NH₂, C₁₋₃ alkyl, —NHC₁₋₃ alkyl, or —N(C₁₋₃ alkyl)₂, wherein the C₁₋₃ alkyl, —NHC₁₋₃ alkyl, and —N(C₁₋₃ alkyl)₂ are each independently and optionally substituted by 1, 2, or 3 R_{d};
R₆ is H or C₁₋₃ alkyl, wherein the C₁₋₃ alkyl is optionally substituted by 1, 2, or 3 Rₑ;
each R₆₁ is independently H, halogen, OH, NH₂, or C₁₋₃ alkyl, wherein the C₁₋₃ alkyl is optionally substituted by 1, 2, or 3 R_{f};
each R₇ is independently H, halogen, OH, NH₂, C₁₋₃ alkyl, —NHC₁₋₃ alkyl, or —N(C₁₋₃ alkyl)₂, wherein the C₁₋₃ alkyl, —NHC₁₋₃ alkyl, and —N(C₁₋₃ alkyl)₂ are each independently and optionally substituted by 1, 2, or 3 R_{g};
or, when E₁ is —C(R₅)₂—, E₂ is —C(R₆₁)₂—, and E₃ is a single bond, R₅ and R₆₁, together with the C atom to which they are attached, make the structural moiety

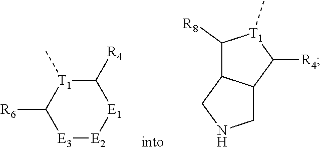

or, when E₂ is —C(R₆₁)₂—, two R₆₁, together with the C atom to which they are attached, make the structural moiety

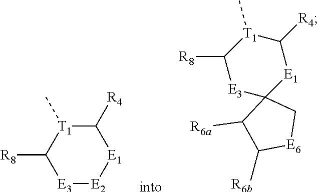

or, when E₃ is a single bond, R₄ and R₈, together with the C atom to which they are attached, make the structural moiety

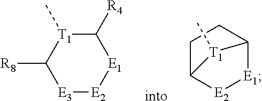

or, when T₁ is CH and E₁ is —C(R₅)₂—, R₅ and R₈, together with the C atom to which they are attached, make the structural moiety

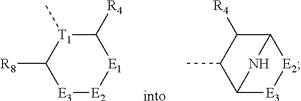

E₆ is NR_{6c} or O;
R₆ₐ and R_{6b} are each independently H, halogen, OH, ═O, NH₂, or C₁₋₃ alkyl;
R_{6c} is H or C₁₋₃ alkyl;
Rₐ, R_{b}, R_{c}, R_{d}, Rₑ, R_{f}, and R_{g} are each independently F, Cl, Br, OH, CN, COOH, or NH₂.

The present disclosure provides a compound of formula (I) or a pharmaceutically acceptable salt thereof,

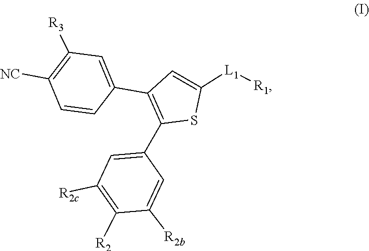

wherein
L₁ is —C(═O)— or —C(═O)—NH—;
R₁ is R₁ₐ or

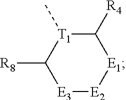

T₁ is CH or N;
E₁ is a single bond, —C(R₅)₂—, or —C(R₅)₂C(R₅)₂—;
E₂ is —NR₆— or —C(R₆₁)₂—;
E₃ is a single bond, —C(R₇)₂—, or —C(R₇)₂C(R₇)₂—;
R₁ₐ is C₁₋₃ alkyl substituted by 1, 2, or 3 Rₐ;
R₂ and R_{2b} are each independently H, halogen, C₁₋₃ alkyl, or C₁₋₃ alkoxy, wherein the C₁₋₃ alkyl and C₁₋₃ alkoxy are optionally substituted by 1, 2, or 3 R_{b};
R_{2c} is H;
or, R₂ and R_{2c}, together with the C atom to which they are attached, make the structural moiety

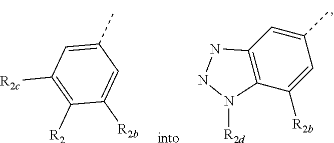

R₂ₐ is H or C₁₋₄ alkyl, wherein the C₁₋₄ alkyl is optionally substituted by 1, 2, or 3 R_{c};
each R₃ is independently H, halogen, or C₁₋₃ alkoxy;
R₄ and R₈ are each H;
each R₅ is independently H, halogen, OH, NH₂, or C₁₋₃ alkyl, wherein the C₁₋₃ alkyl is optionally substituted by 1, 2, or 3 R_{d};
R₆ is H or C₁₋₃ alkyl, wherein the C₁₋₃ alkyl is optionally substituted by 1, 2, or 3 Rₑ;
each R₆₁ is independently H, halogen, OH, NH₂, or C₁₋₃ alkyl, wherein the C₁₋₃ alkyl is optionally substituted by 1, 2, or 3 R_{f};
each R₇ is independently H, halogen, OH, NH₂, or C₁₋₃ alkyl, wherein the C₁₋₃ alkyl is optionally substituted by 1, 2, or 3 R_{g};
or, when E₁ is —C(R₅)₂—, E₂ is —C(R₆₁)₂—, and E₃ is a single bond, R₅ and R₆₁, together with the C atom to which they are attached, make the structural moiety

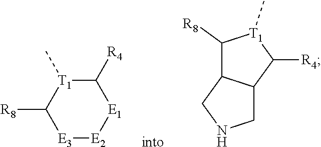

or, when E₂ is —C(R₆₁)₂—, two R₆₁, together with the C atom to which they are attached, make the structural moiety

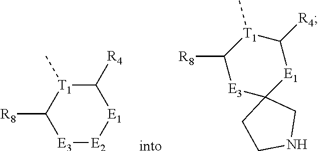

or, when E₃ is a single bond, R₄ and R₈, together with the C atom to which they are attached, make the structural moiety

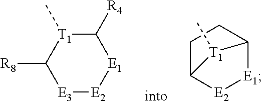

or, when T₁ is CH and E₁ is —C(R₅)₂—, R₅ and R₈, together with the C atom to which they are attached, make the structural moiety

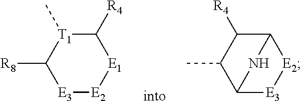

Rₐ, R_{b}, R_{c}, R_{d}, Rₑ, R_{f}, and R_{g} are each independently F, Cl, Br, OH, or NH₂.

In some embodiments of the present disclosure, the compound or the pharmaceutically acceptable salt thereof, wherein the compound is

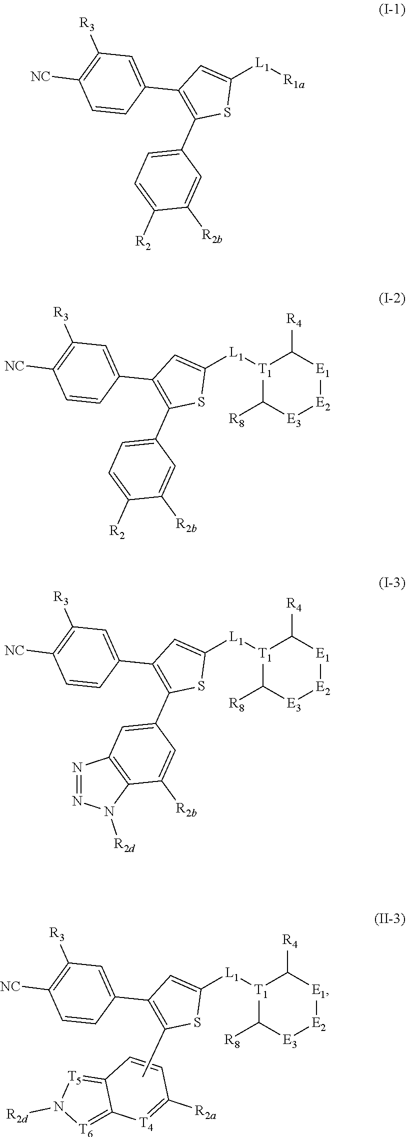

wherein
R₂ is H, halogen, NH₂, C₁₋₃ alkyl, C₁₋₃ alkoxy, —S(═O)₂C₁₋₃ alkyl, —NHC₁₋₃ alkyl, 4- to 6-membered heterocycloalkyl, —C(═O)-4- to 6-membered heterocycloalkyl, or —NHC₁₋₃ alkyl-C₃₋₆ cycloalkyl, wherein the C₁₋₃ alkyl, C₁₋₃ alkoxy, —S(═O)₂C₁₋₃ alkyl, —NHC₁₋₃ alkyl, 4- to 6-membered heterocycloalkyl, —C(═O)-4- to 6-membered heterocycloalkyl, and —NHC₁₋₃ alkyl-C₃₋₆ cycloalkyl are each independently and optionally substituted by 1, 2, or 3 R_{b};
R_{b}, R₁ₐ, R₂ₐ, R_{2b}, R₂ₐ, R₃, R₄, R₈, L₁, T₁, T₄, T₅, T₆, E₁, E₂, and E₃ are as defined in the present disclosure.

In some embodiments of the present disclosure, the compound or the pharmaceutically acceptable salt thereof, wherein the compound is

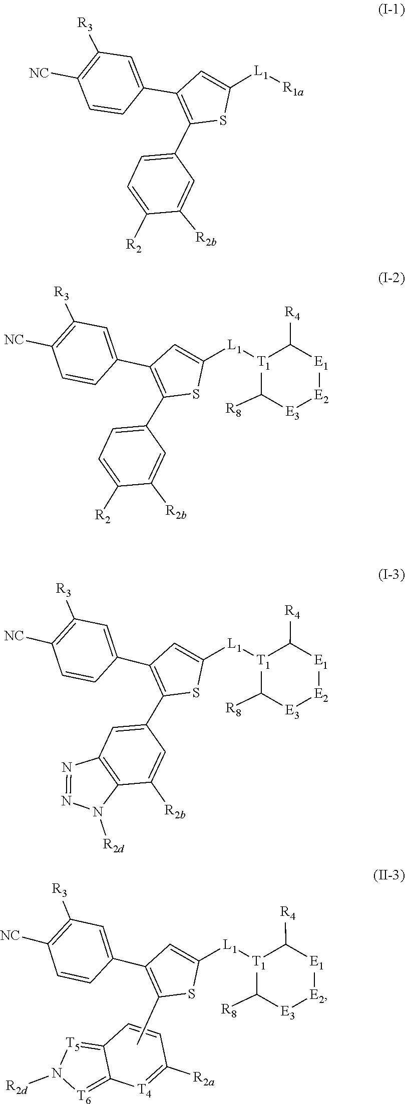

wherein
R₂ and R_{2b} are each independently H, halogen, NH₂, C₁₋₃ alkyl, C₁₋₃ alkoxy, —S(═O)₂C₁₋₃ alkyl, 4- to 6-membered heterocycloalkyl, or —C(═O)-4- to 6-membered heterocycloalkyl, wherein the C₁₋₃ alkyl, C₁₋₃ alkoxy, —S(═O)₂C₁₋₃ alkyl, 4- to 6-membered heterocycloalkyl, and —C(═O)-4- to 6-membered heterocycloalkyl are each independently and optionally substituted by 1, 2, or 3 R_{b};
R_{b}, R₁ₐ, R₂ₐ, R_{2d}, R₃, R₄, R₈, L₁, T₁, T₄, T₅, T₆, E₁, E₂, and E₃ are as defined in the present disclosure.

In some embodiments of the present disclosure, the compound or the pharmaceutically acceptable salt thereof, wherein the compound is

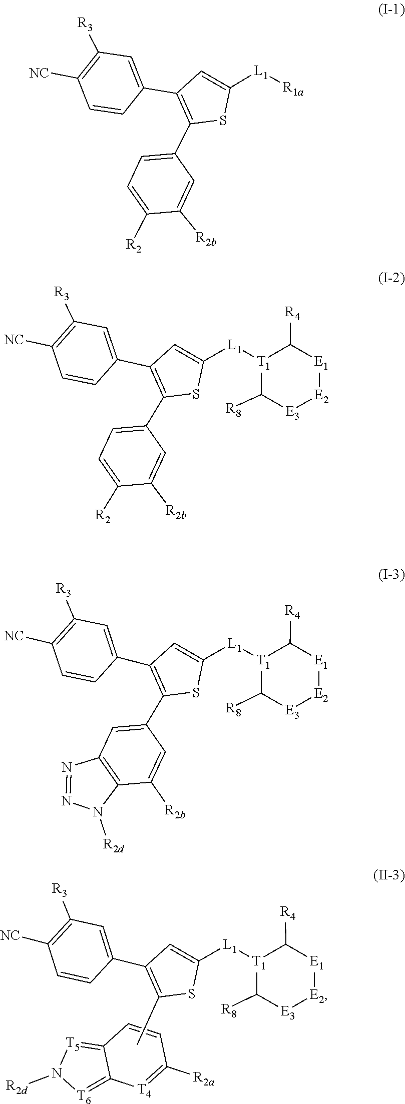

wherein
R₂ and R_{2b} are each independently H, halogen, NH₂, C₁₋₃ alkyl, C₁₋₃ alkoxy, or —S(═O)₂C₁₋₃ alkyl, wherein the C₁₋₃ alkyl, C₁₋₃ alkoxy, and —S(═O)₂C₁₋₃ alkyl are each independently and optionally substituted by 1, 2, or 3 R_{b};
R_{b}, R₁ₐ, R₂ₐ, R_{2d}, R₃, R₄, R₈, L₁, T₁, T₄, T₅, T₆, E₁, E₂, and E₃ are as defined in the present disclosure.

In some embodiments of the present disclosure, the compound or the pharmaceutically acceptable salt thereof, wherein the compound is

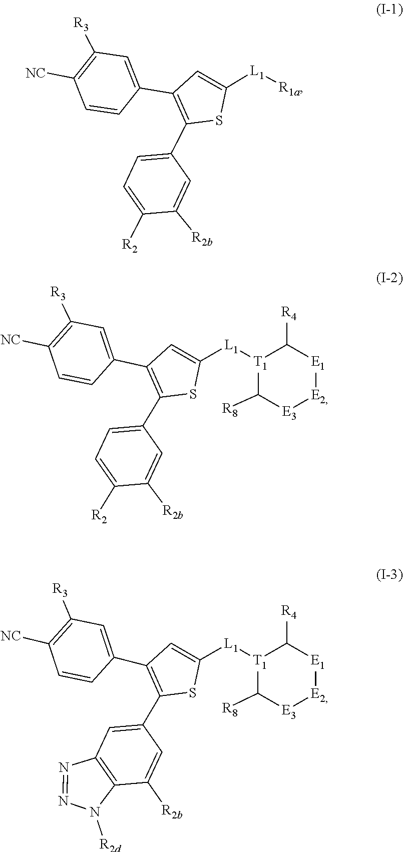

wherein
R₂ and R_{2b} are each independently H, halogen, C₁₋₃ alkyl, or C₁₋₃ alkoxy, wherein the C₁₋₃ alkyl and C₁₋₃ alkoxy are optionally substituted by 1, 2, or 3 R_{b};
R_{b}, R₁ₐ, R₂ₐ, R₃, R₄, R₈, L₁, T₁, E₁, E₂, and E₃ are as defined in the present disclosure.

In some embodiments of the present disclosure, the Rₐ, R_{b}, R_{c}, R_{d}, Rₑ, R_{f}, R_{g}, and Rₜ are each independently F, Cl, Br, OH, CN, COOH, NH₂, CH₃, or OCH₃, and other variables are as defined in the present disclosure.

In some embodiments of the present disclosure, the Rₐ, R_{b}, R_{c}, R_{d}, Rₑ, R_{f}, and R_{g} are each independently F, Cl, Br, OH, CN, COOH, NH₂, CH₃, or OCH₃, and other variables are as defined in the present disclosure.

In some embodiments of the present disclosure, each Rₜ is independently H, F, Cl, or CH₃, and other variables are as defined in the present disclosure.

In some embodiments of the present disclosure, the R₁ₐ is

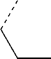

substituted by 1, 2, or 3 Rₐ, and Rₐ and other variables are as defined in the present disclosure.

In some embodiments of the present disclosure, the R₁ₐ is

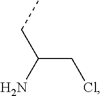

and other variables are as defined in the present disclosure.

In some embodiments of the present disclosure, the R₂ₐ is H or F, and other variables are as defined in the present disclosure.

In some embodiments of the present disclosure, the R₂ and R_{2b} are each independently H, F, Cl, NH₂, —OCH₃,

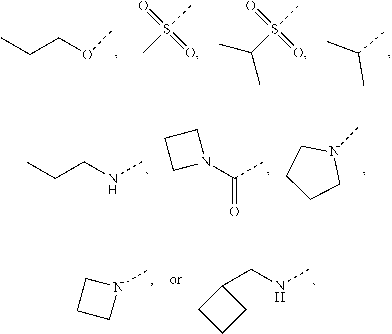

wherein the —OCH₃,

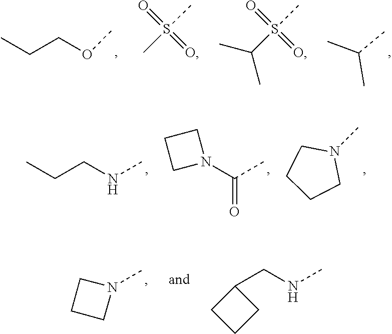

are each independently and optionally substituted by 1, 2, or 3 R_{b}, and R_{b} and other variables are as defined in the present disclosure.

In some embodiments of the present disclosure, the R₂ and R_{2b} are each independently H, F, Cl, NH₂, —OCH₃,

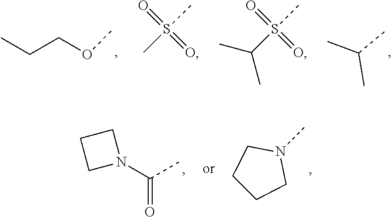

wherein the —OCH₃,

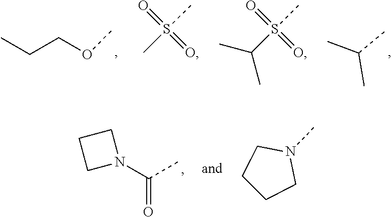

are each independently and optionally substituted by 1, 2, or 3 R_{b}, and R_{b} and other variables are as defined in the present disclosure.

In some embodiments of the present disclosure, the R₂ and R_{2b} are each independently H, F, Cl, NH₂, —OCH₃,

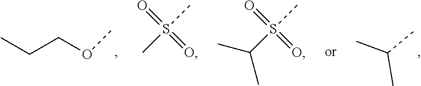

wherein the —OCH₃,

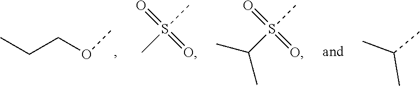

are each independently and optionally substituted by 1, 2, or 3 R_{b}, and R_{b} and other variables are as defined in the present disclosure.

In some embodiments of the present disclosure, the R₂ and R_{2b} are each independently H, F, Cl, or —OCH₃, wherein the —OCH₃ is optionally substituted by 1, 2, or 3 R_{b}, and R_{b} and other variables are as defined in the present disclosure.

In some embodiments of the present disclosure, the R₂ and R_{2b} are each independently H, F, Cl, NH₂, —OCH₃,

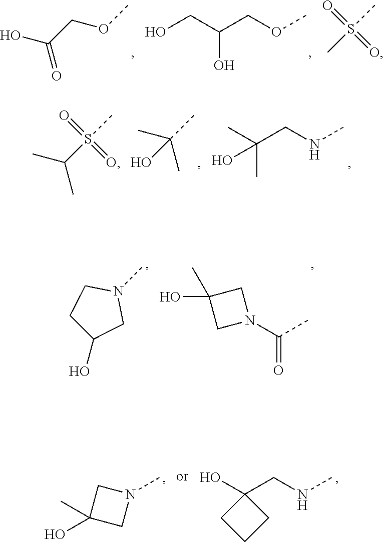

and other variables are as defined in the present disclosure.

In some embodiments of the present disclosure, the R₂ and R_{2b} are each independently H, F, Cl, NH₂, —OCH₃,

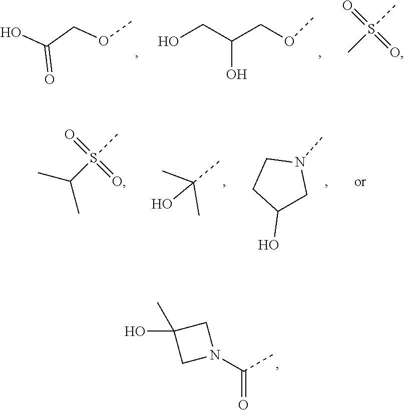

and other variables are as defined in the present disclosure.

In some embodiments of the present disclosure, the R₂ and R_{2b} are each independently H, F, Cl, NH₂, —OCH₃,

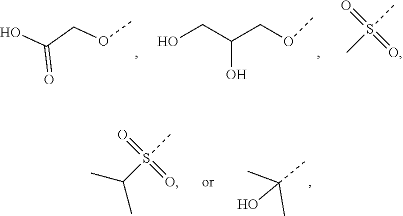

and other variables are as defined in the present disclosure.

In some embodiments of the present disclosure, the R₂ and R_{2b} are each independently F, Cl, or —OCH₃, and other variables are as defined in the present disclosure.

In some embodiments of the present disclosure, the R₂ₐ is H, CH₃,

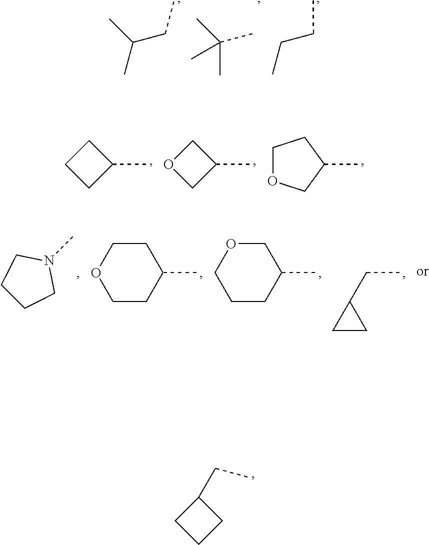

wherein the —CH₃,

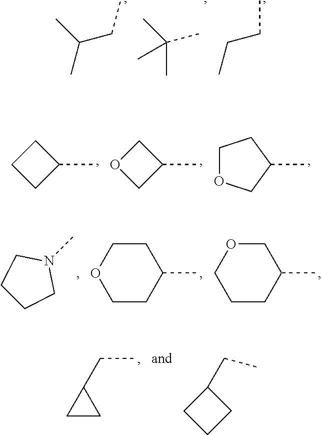

are optionally substituted by 1, 2, or 3 R_{c}, and R_{c} and other variables are as defined in the present disclosure.

In some embodiments of the present disclosure, the R₂ is H, CH₃,

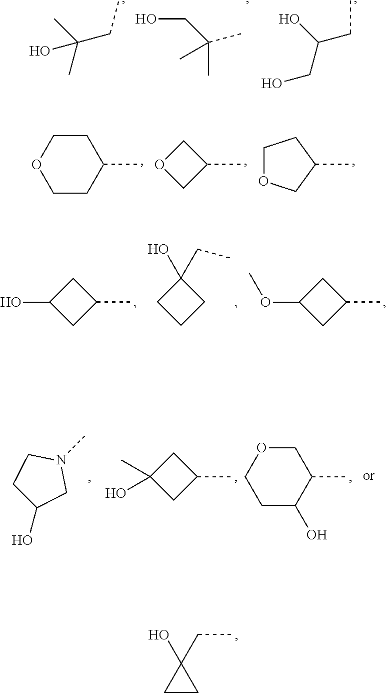

and other variables are as defined in the present disclosure.

In some embodiments of the present disclosure, the R₂ₐ is H, CH₃, or

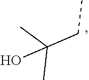

and other variables are as defined in the present disclosure.

In some embodiments of the present disclosure, the R_{2d} is CH₃ or

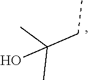

and other variables are as defined in the present disclosure.

In some embodiments of the present disclosure, the R₂ₑ, R_{2f}, and R_{2g} are each independently H or CH₃, and other variables are as defined in the present disclosure.

In some embodiments of the present disclosure, each R₃ is independently F or OCH₃, and other variables are as defined in the present disclosure.

In some embodiments of the present disclosure, each R₃ is independently F, and other variables are as defined in the present disclosure.

In some embodiments of the present disclosure, the R₄ and R₈ are each independently H or CH₃, and other variables are as defined in the present disclosure.

In some embodiments of the present disclosure, each R₅ is independently H, F, Cl, Br, I, OH, NH₂, CH₃, —NH—CH₃, or

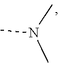

wherein the CH₃, —NH—CH₃, and

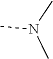

are each independently and optionally substituted by 1, 2, or 3 R_{d}, and R_{d} and other variables are as defined in the present disclosure.

In some embodiments of the present disclosure, each R₅ is independently H, F, Cl, Br, I, OH, NH₂, or CH₃, wherein the CH₃ is optionally substituted by 1, 2, or 3 R_{d}, and R_{d} and other variables are as defined in the present disclosure.

In some embodiments of the present disclosure, each R₅ is independently H, OH, NH₂, CH₃, or

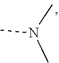

and other variables are as defined in the present disclosure.

In some embodiments of the present disclosure, each R₅ is independently H or NH₂, and other variables are as defined in the present disclosure.

In some embodiments of the present disclosure, the R₆ is H, CH₃, or CH₂—CH₃, wherein the CH₃ and CH₂—CH₃ are each independently and optionally substituted by 1, 2, or 3 Rₑ, and Rₑ and other variables are as defined in the present disclosure.

In some embodiments of the present disclosure, the R₆ is H or CH₃, wherein the CH₃ is optionally substituted by 1, 2, or 3 Rₑ, and other variables are as defined in the present disclosure.

In some embodiments of the present disclosure, the R₆ is H, CH₂—CN, or CH₂—CF₃, and other variables are as defined in the present disclosure.

In some embodiments of the present disclosure, the R₆ is H, and other variables are as defined in the present disclosure.

In some embodiments of the present disclosure, each R₆₁ is independently H, F, Cl, Br, I, OH, NH₂, or CH₃, wherein the CH₃ is optionally substituted by 1, 2, or 3 R_{f}, and R_{f} and other variables are as defined in the present disclosure.

In some embodiments of the present disclosure, each R₆₁ is independently H, F, OH, or NH₂, and other variables are as defined in the present disclosure.

In some embodiments of the present disclosure, each R₆₁ is independently H or NH₂, and other variables are as defined in the present disclosure.

In some embodiments of the present disclosure, the R₆ₐ and R_{6b} are each independently H, OH, ═O, NH₂, or CH₃, and other variables are as defined in the present disclosure.

In some embodiments of the present disclosure, each R₇ is independently H, F, Cl, Br, I, OH, NH₂, CH₃, —NH—CH₃, or

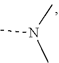

wherein the CH₃, —NH—CH₃, and

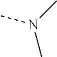

are optionally substituted by 1, 2, or 3 R_{g}, and R_{g} and other variables are as defined in the present disclosure.

In some embodiments of the present disclosure, each R₇ is independently H, F, Cl, Br, I, OH, NH₂, or CH₃, wherein the —CH₃ is optionally substituted by 1, 2, or 3 R_{g}, and R_{g} and other variables are as defined in the present disclosure.

In some embodiments of the present disclosure, each R₇ is independently H, OH, NH₂, CH₃, or

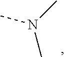

and other variables are as defined in the present disclosure.

In some embodiments of the present disclosure, each R₇ is independently H or NH₂, and other variables are as defined in the present disclosure.

In some embodiments of the present disclosure, the structural moiety

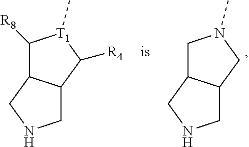

and other variables are as defined in the present disclosure.

In some embodiments of the present disclosure, the structural moiety

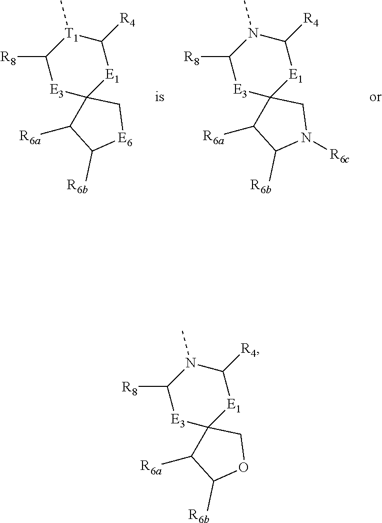

and other variables are as defined in the present disclosure.

In some embodiments of the present disclosure, the structural moiety

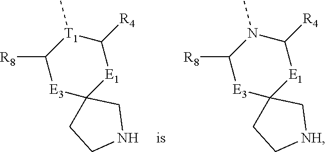

and other variables are as defined in the present disclosure.

In some embodiments of the present disclosure, the structural moiety

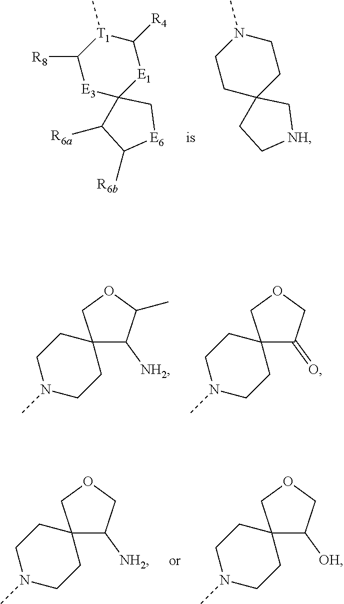

and other variables are as defined in the present disclosure.

In some embodiments of the present disclosure, the structural moiety

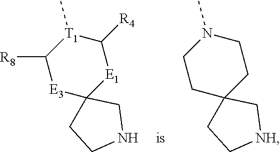

and other variables are as defined in the present disclosure.

In some embodiments of the present disclosure, the structural moiety

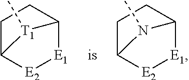

and other variables are as defined in the present disclosure.

In some embodiments of the present disclosure, the structural moiety

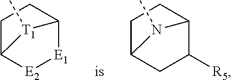

and other variables are as defined in the present disclosure.

In some embodiments of the present disclosure, the structural moiety

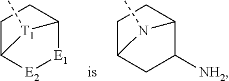

and other variables are as defined in the present disclosure.

In some embodiments of the present disclosure, the structural moiety

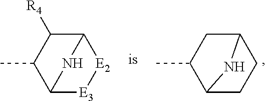

and other variables are as defined in the present disclosure.

In some embodiments of the present disclosure, the R₁ is

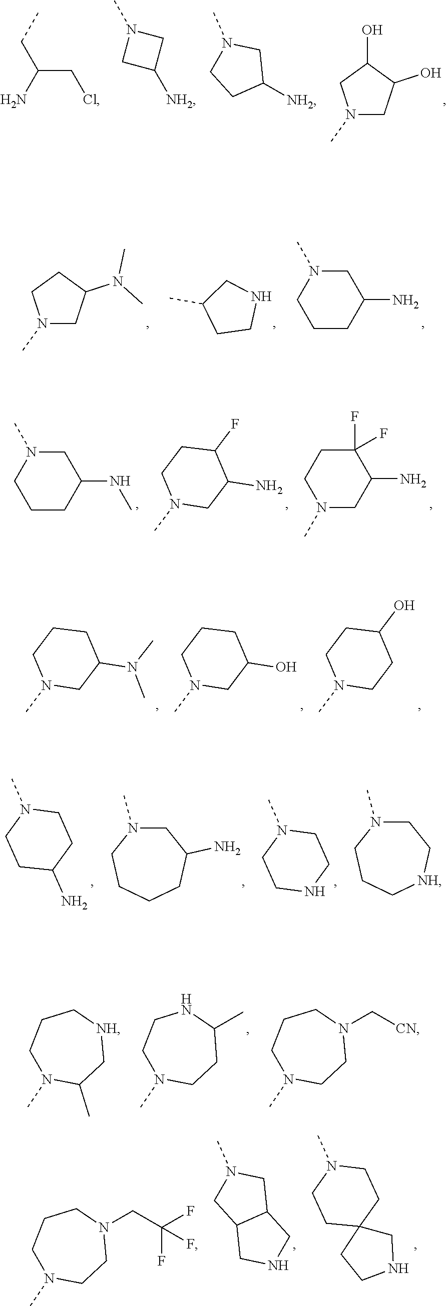

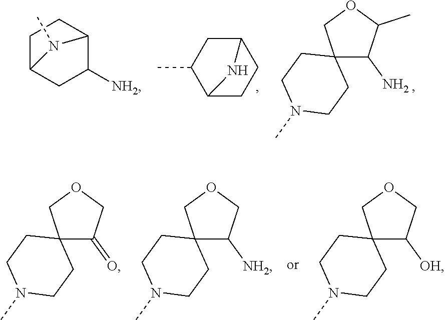

and other variables are as defined in the present disclosure.

In some embodiments of the present disclosure, the R₁ is

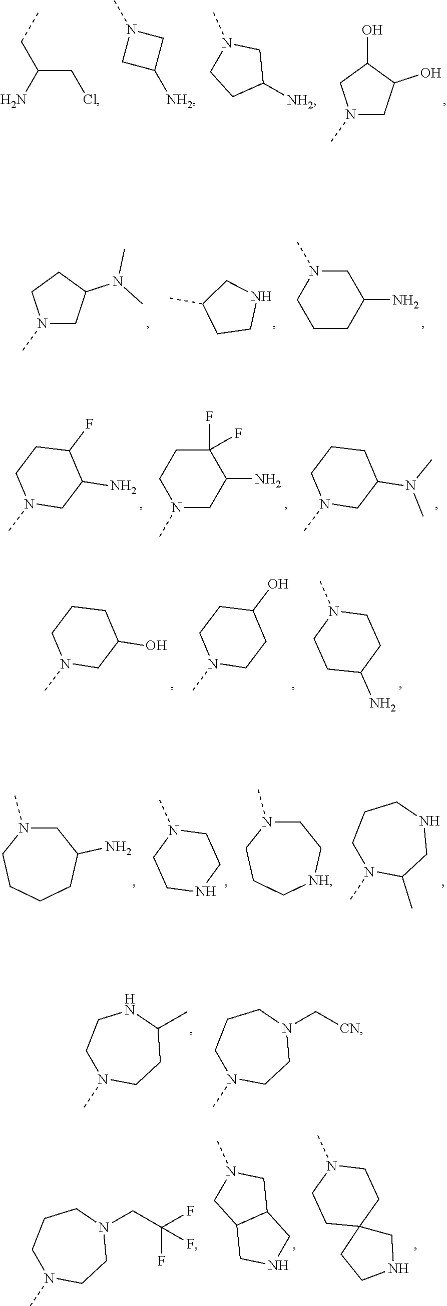

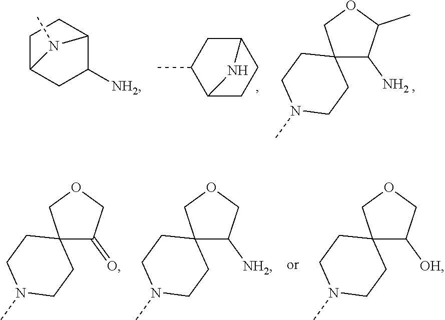

and other variables are as defined in the present disclosure.

In some embodiments of the present disclosure, the R₁ is

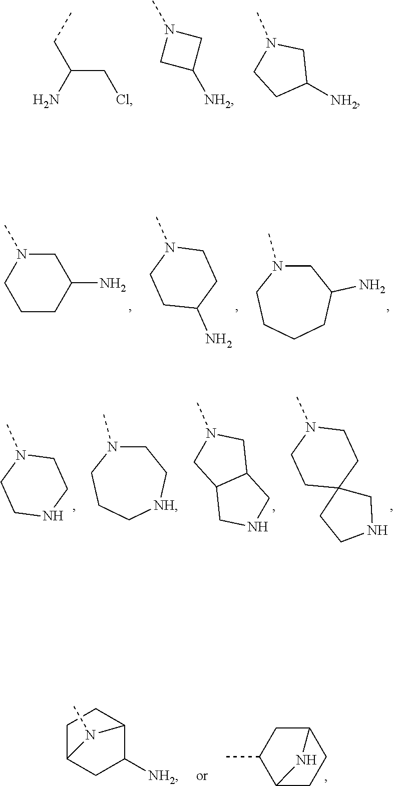

and other variables are as defined in the present disclosure.

In some embodiments of the present disclosure, the ring A is

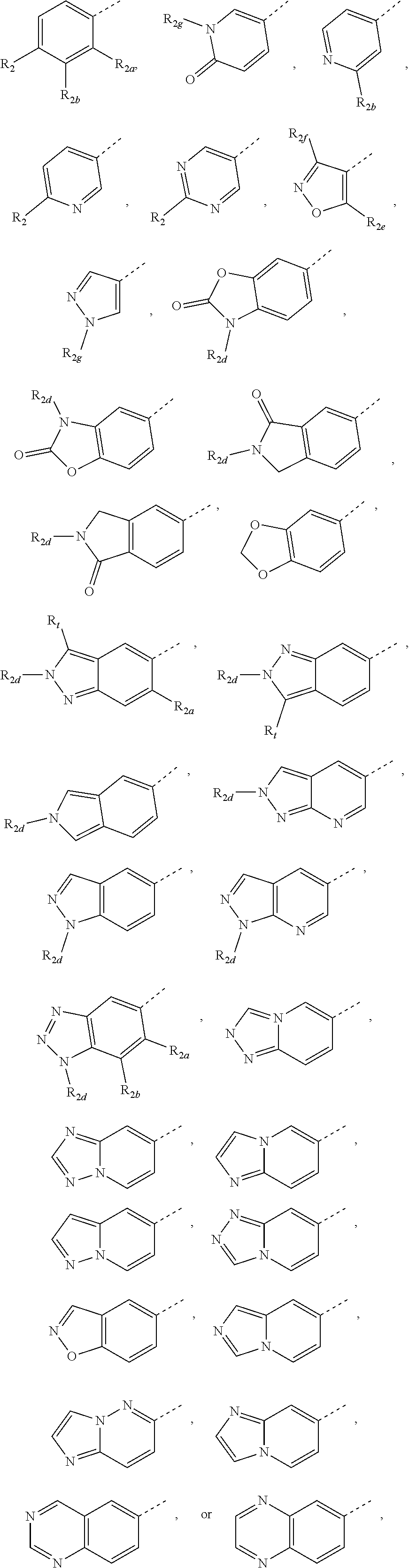

and R₂, R₂ₐ, R_{2b}, R_{2d}, R₂ₑ, R_{2f}, R_{2g}, Rₜ, and other variables are as defined in the present disclosure.

In some embodiments of the present disclosure, the ring A is

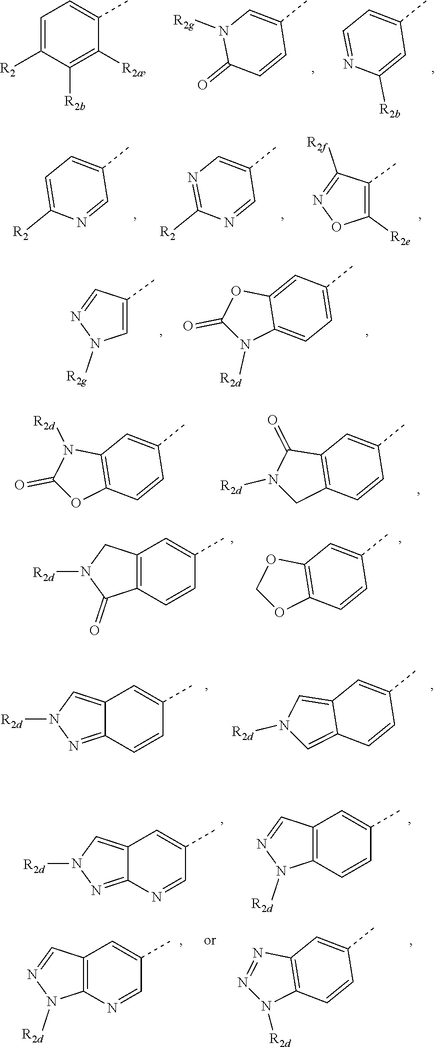

and R₂, R₂ₐ, R_{2b}, R₂ₐ, R₂ₑ, R_{2f}, R_{2g}, Rₜ, and other variables are as defined in the present disclosure.

In some embodiments of the present disclosure, the ring A is

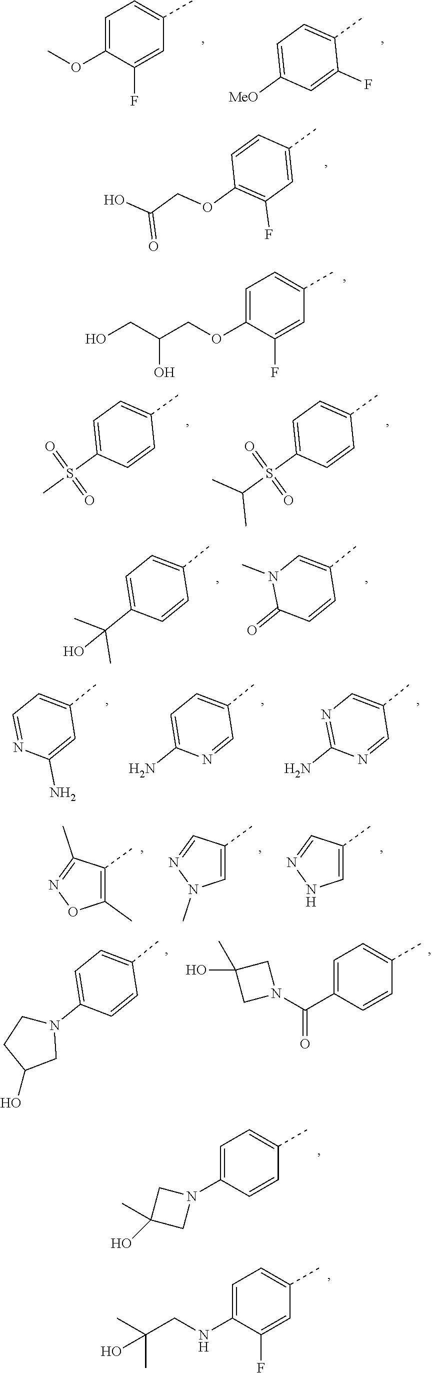

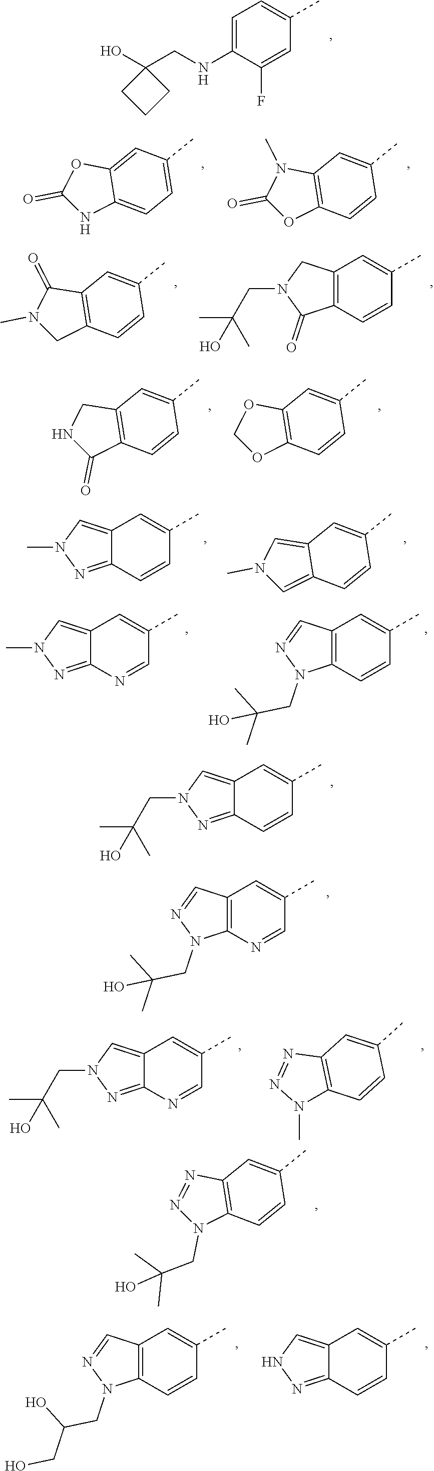

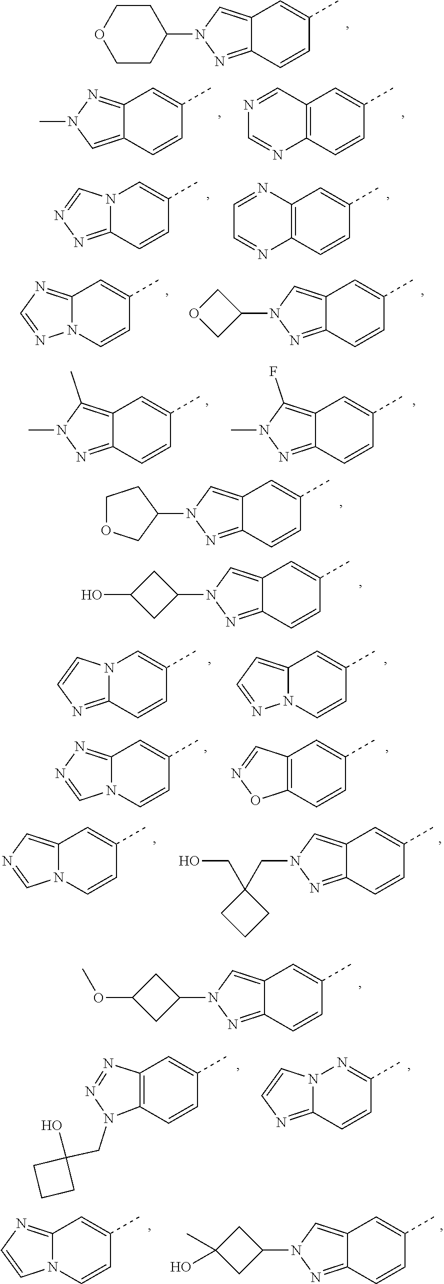

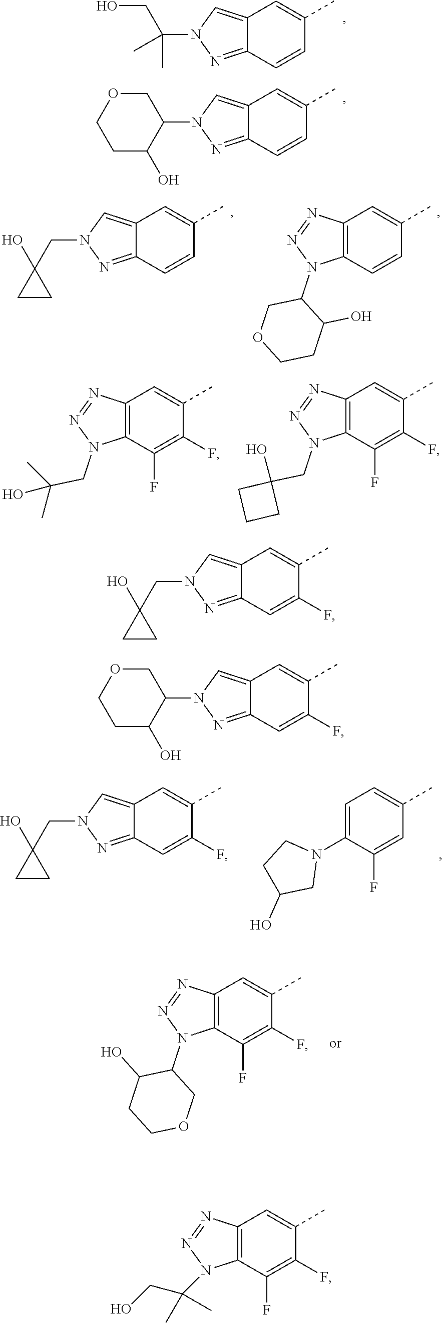

and other variables are as defined in the present disclosure.

In some embodiments of the present disclosure, the ring A is

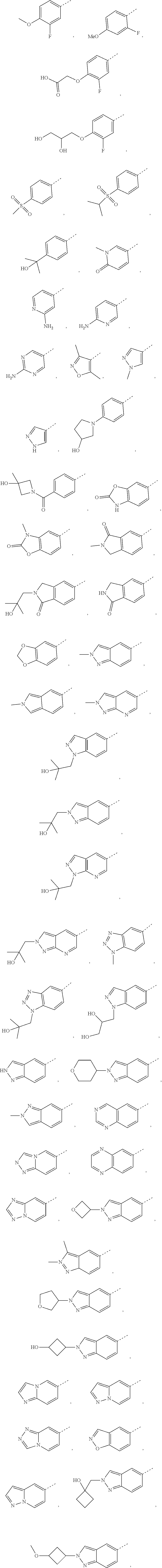

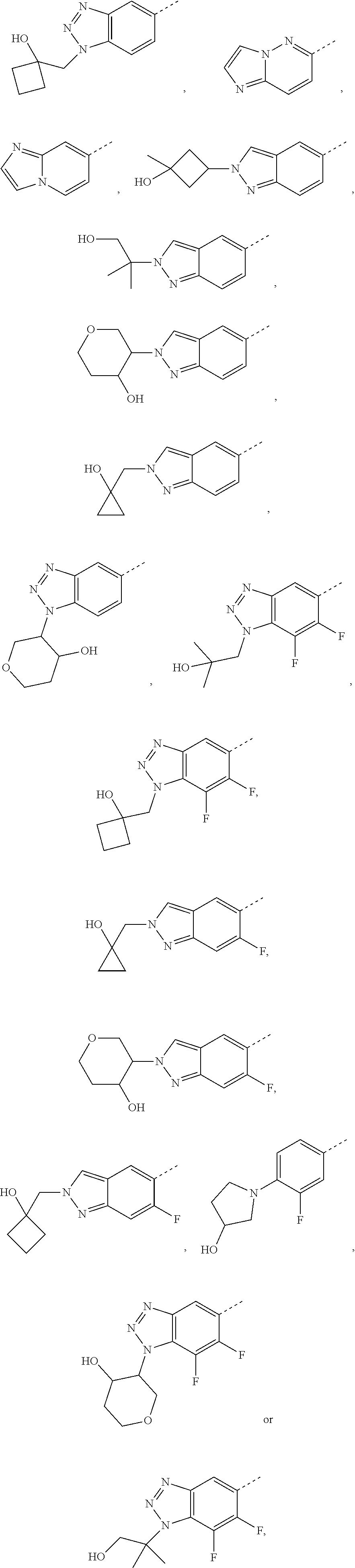

and other variables are as defined in the present disclosure.

In some embodiments of the present disclosure, the ring A is

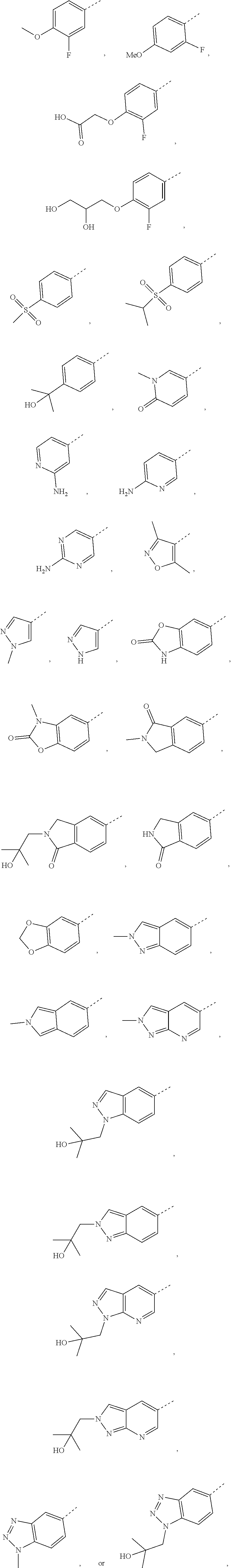

and other variables are as defined in the present disclosure.

In some embodiments of the present disclosure, the compound or the pharmaceutically acceptable salt thereof, wherein the compound is

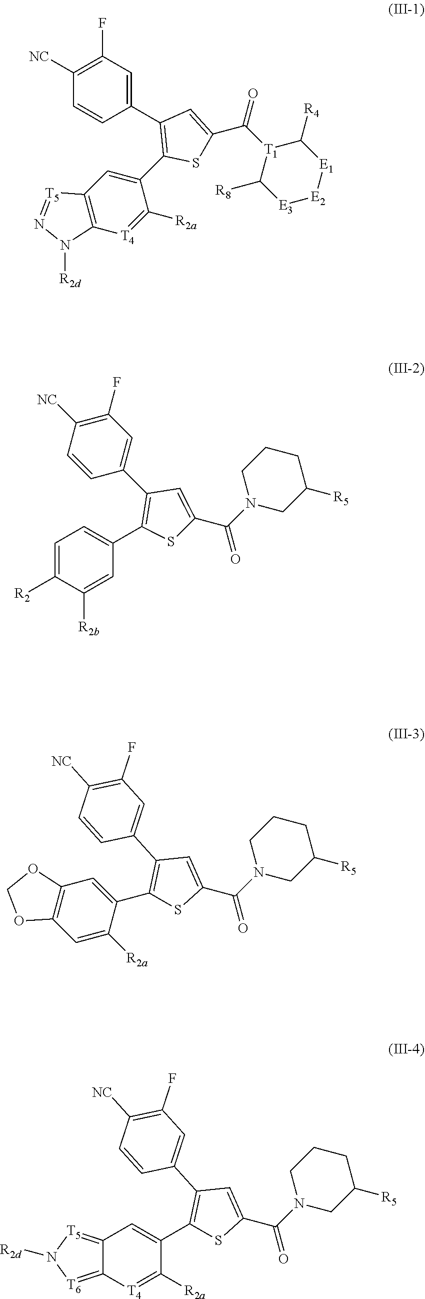

wherein
R₂ and R_{2b} are each independently H, halogen, NH₂, C₁₋₃ alkyl, C₁₋₃ alkoxy, —S(═O)₂C₁₋₃ alkyl, 4- to 6-membered heterocycloalkyl, or —C(═O)-4- to 6-membered heterocycloalkyl, wherein the C₁₋₃ alkyl, C₁₋₃ alkoxy, —S(═O)₂C₁₋₃ alkyl, 4- to 6-membered heterocycloalkyl, and —C(═O)-4- to 6-membered heterocycloalkyl are each independently and optionally substituted by 1, 2, or 3 R_{b};
R_{b}, R₂ₐ, R_{2d}, R₄, R₅, R₈, T₁, T₄, T₅, T₆, E₁, E₂, and E₃ are as defined in the present disclosure.

In some embodiments of the present disclosure, the compound or the pharmaceutically acceptable salt thereof, wherein the compound is

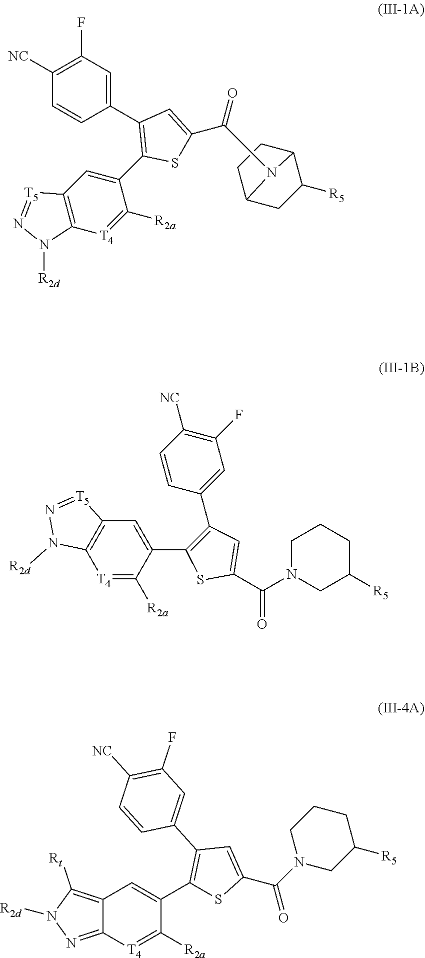

wherein R₂ₐ, R_{2d}, Rₜ, R₈, T₄, and T₅ are as defined in the present disclosure.

In some embodiments of the present disclosure, the compound or the pharmaceutically acceptable salt thereof, wherein the compound is

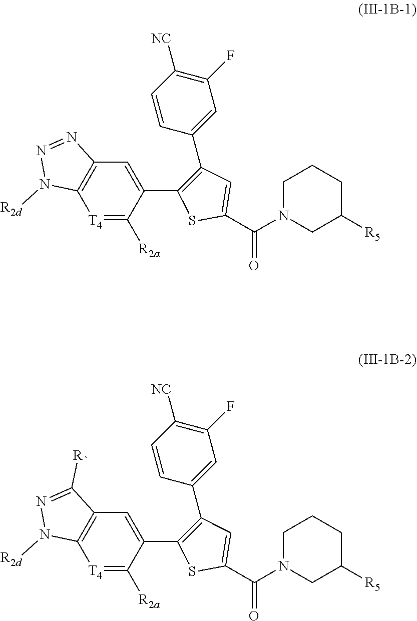

wherein R₂ₐ, R_{2d}, Rₜ, T₄, and R₅ are as defined in the present disclosure.

There are still some embodiments of the present disclosure which are obtained by any combination of the above variables.

The present disclosure also provides a compound of the following formula or a pharmaceutically acceptable salt thereof,

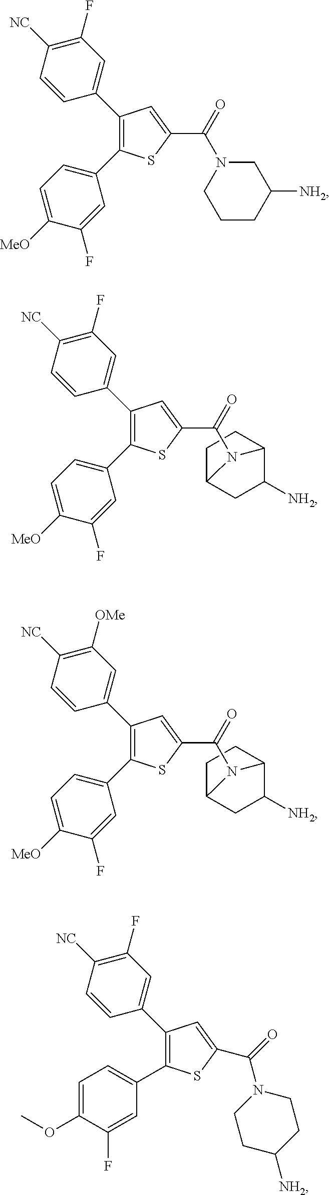

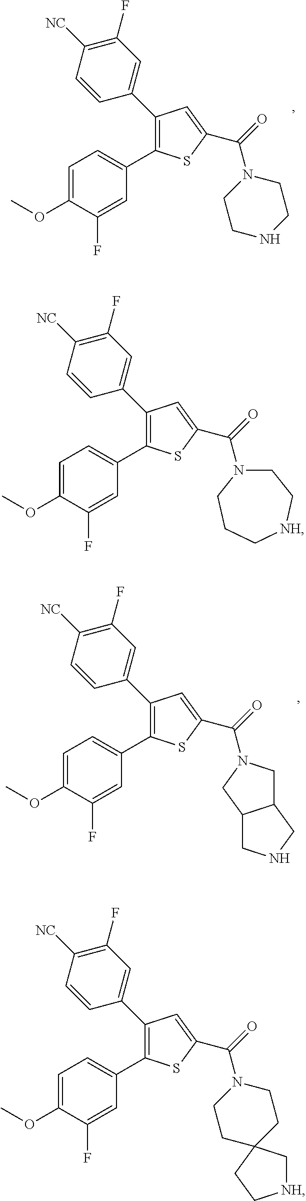 [SMILES: COc1ccc(-c2sc(C(=O)N3CCNCC3)cc2-c2ccc(C#N)c(F)c2)cc1F ; COc1ccc(-c2sc(C(=O)N3CCCNCC3)cc2-c2ccc(C#N)c(F)c2)cc1F ; COc1ccc(-c2sc(C(=O)N3CC4CNCC4C3)cc2-c2ccc(C#N)c(F)c2)cc1F ; COc1ccc(-c2sc(C(=O)N3CCC4(CCNC4)CC3)cc2-c2ccc(C#N)c(F)c2)cc1F]

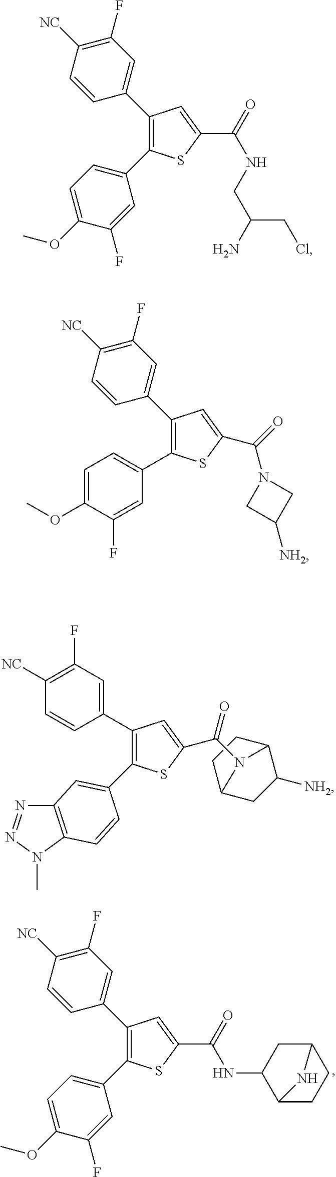

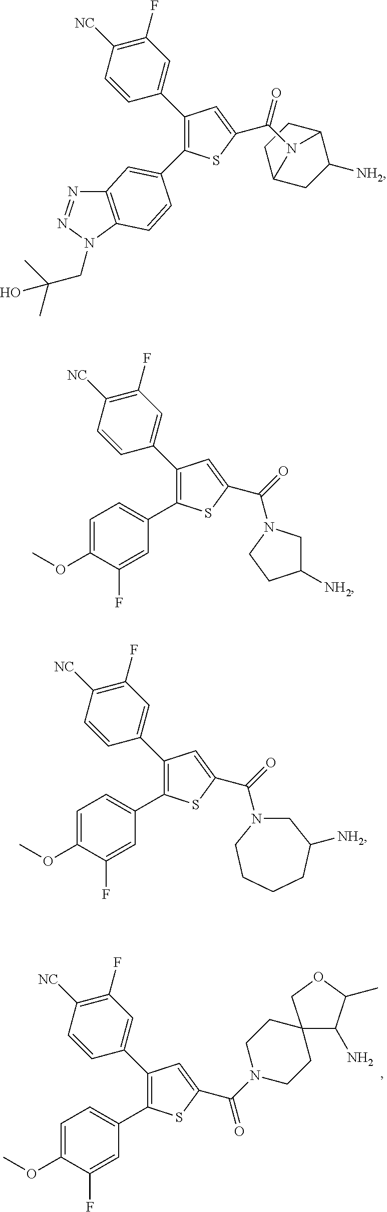

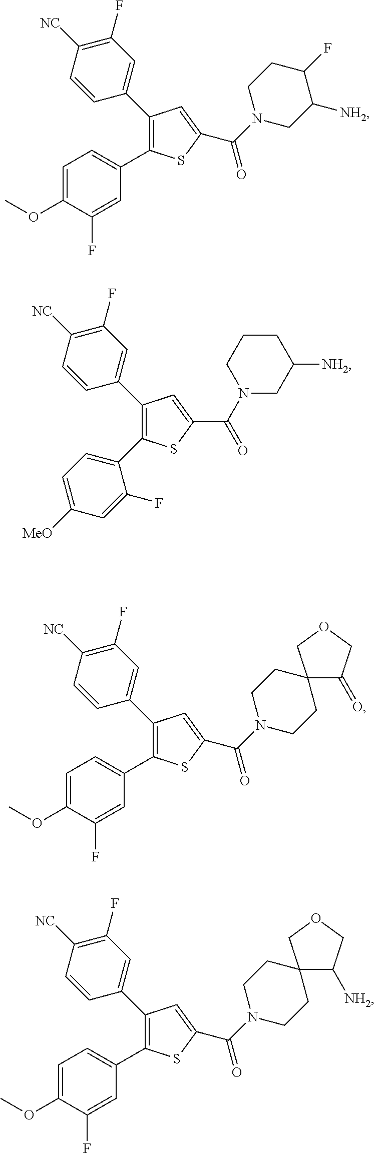 [SMILES: COc1ccc(-c2sc(C(=O)N3CCC(F)C(N)C3)cc2-c2ccc(C#N)c(F)c2)cc1F ; COc1ccc(-c2sc(C(=O)N3CCCC(N)C3)cc2-c2ccc(C#N)c(F)c2)c(F)c1 ; COc1ccc(-c2sc(C(=O)N3CCC4(CC3)COCC4=O)cc2-c2ccc(C#N)c(F)c2)cc1F ; COc1ccc(-c2sc(C(=O)N3CCC4(CC3)COCC4N)cc2-c2ccc(C#N)c(F)c2)cc1F]

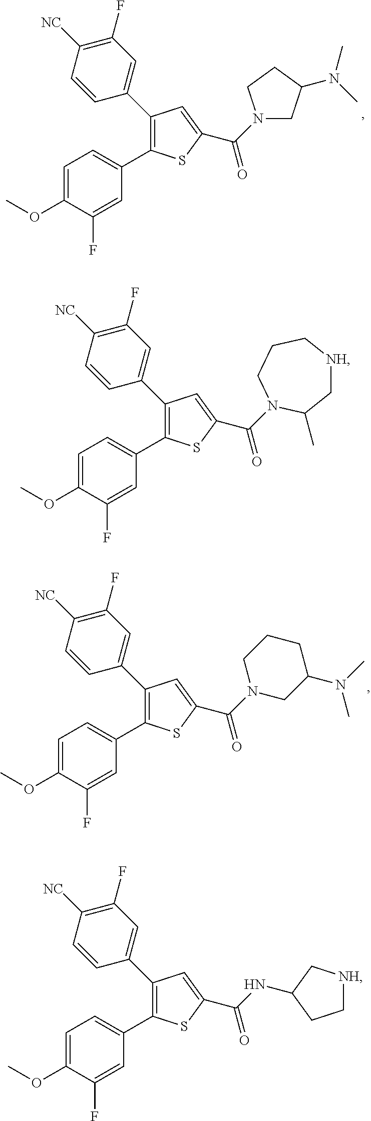

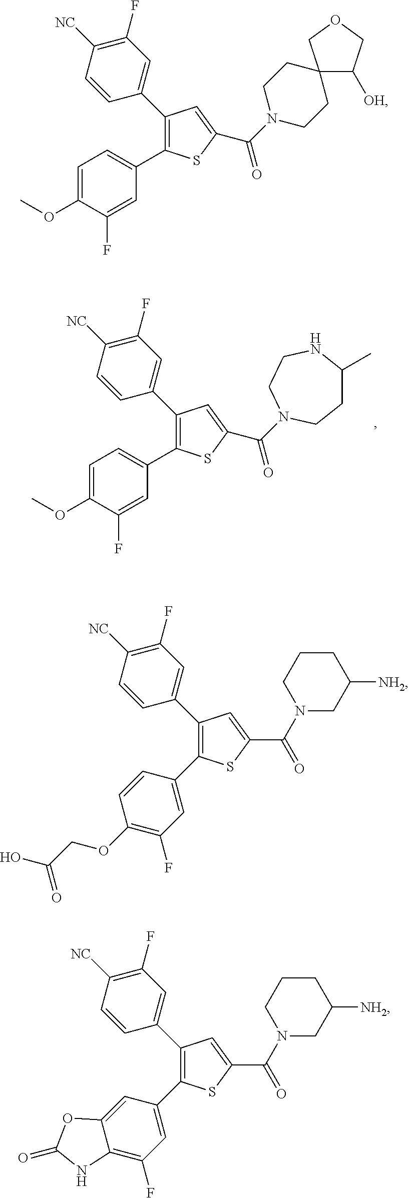

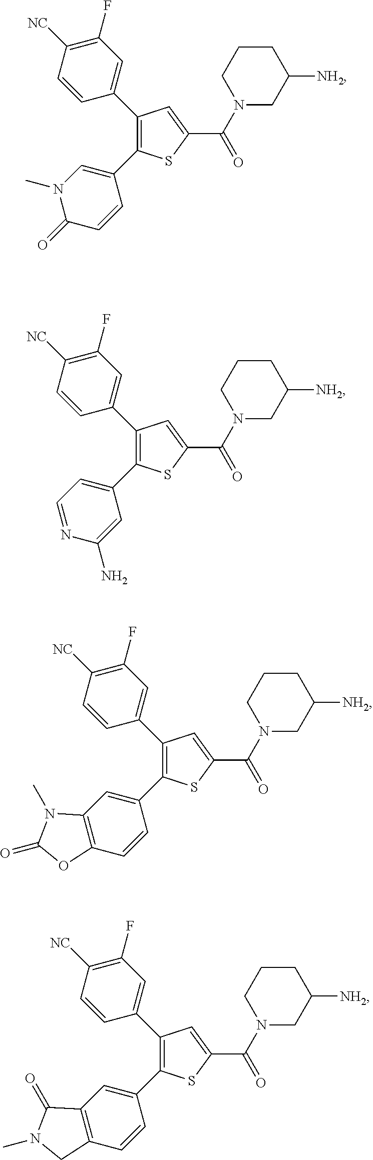 [SMILES: Cn1cc(-c2sc(C(=O)N3CCCC(N)C3)cc2-c2ccc(C#N)c(F)c2)ccc1=O ; N#Cc1ccc(-c2cc(C(=O)N3CCCC(N)C3)sc2-c2ccnc(N)c2)cc1F ; Cn1c(=O)oc2ccc(-c3sc(C(=O)N4CCCC(N)C4)cc3-c3ccc(C#N)c(F)c3)cc21 ; CN1Cc2ccc(-c3sc(C(=O)N4CCCC(N)C4)cc3-c3ccc(C#N)c(F)c3)cc2C1=O]

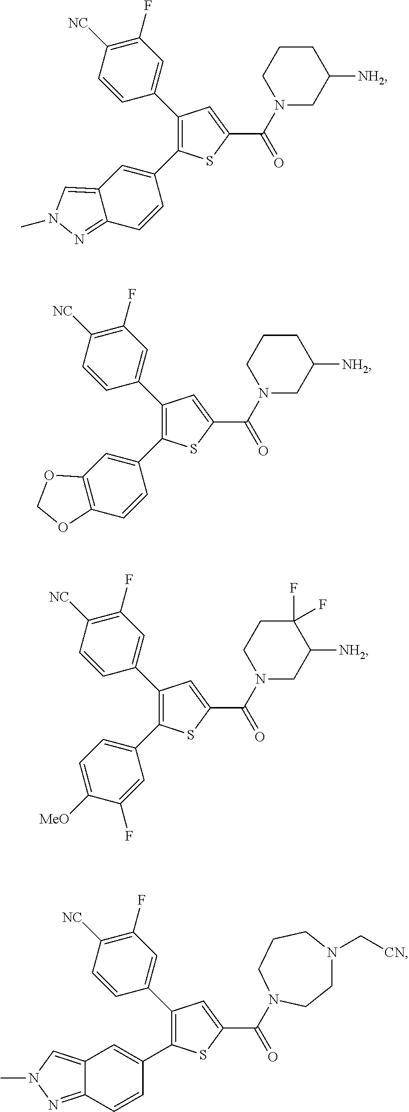 [SMILES: Cn1cc2cc(-c3sc(C(=O)N4CCCC(N)C4)cc3-c3ccc(C#N)c(F)c3)ccc2n1 ; N#Cc1ccc(-c2cc(C(=O)N3CCCC(N)C3)sc2-c2ccc3c(c2)OCO3)cc1F ; COc1ccc(-c2sc(C(=O)N3CCC(F)(F)C(N)C3)cc2-c2ccc(C#N)c(F)c2)cc1F ; Cn1cc2cc(-c3sc(C(=O)N4CCCN(CC#N)CC4)cc3-c3ccc(C#N)c(F)c3)ccc2n1]

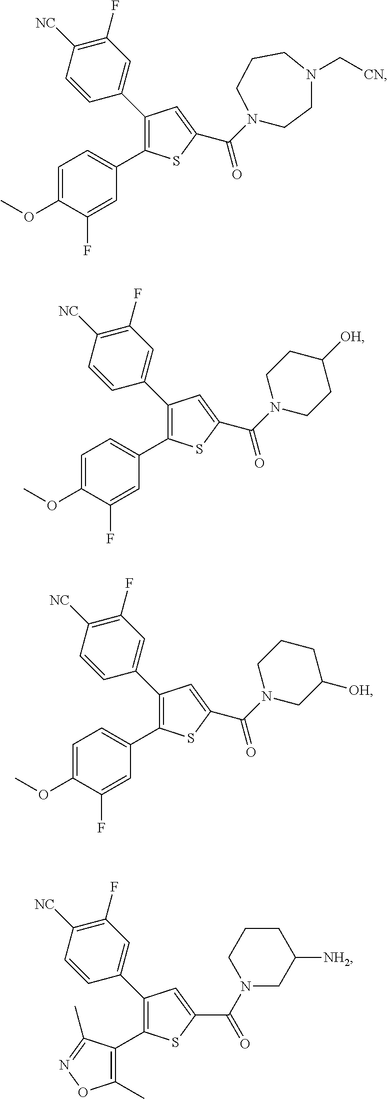 [SMILES: COc1ccc(-c2sc(C(=O)N3CCCN(CC#N)CC3)cc2-c2ccc(C#N)c(F)c2)cc1F ; COc1ccc(-c2sc(C(=O)N3CCC(O)CC3)cc2-c2ccc(C#N)c(F)c2)cc1F ; COc1ccc(-c2sc(C(=O)N3CCCC(O)C3)cc2-c2ccc(C#N)c(F)c2)cc1F ; Cc1noc(C)c1-c1sc(C(=O)N2CCCC(N)C2)cc1-c1ccc(C#N)c(F)c1]

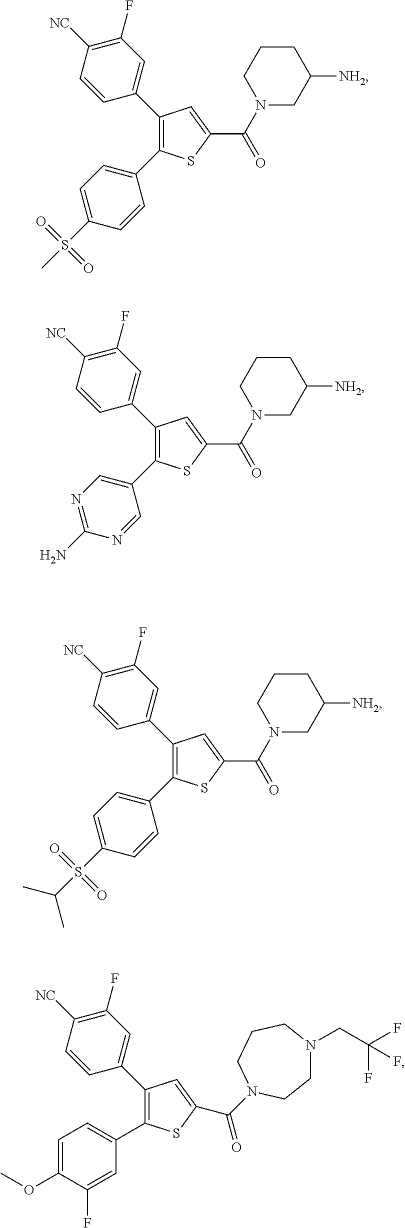 [SMILES: CS(=O)(=O)c1ccc(-c2sc(C(=O)N3CCCC(N)C3)cc2-c2ccc(C#N)c(F)c2)cc1 ; N#Cc1ccc(-c2cc(C(=O)N3CCCC(N)C3)sc2-c2cnc(N)nc2)cc1F ; CC(C)S(=O)(=O)c1ccc(-c2sc(C(=O)N3CCCC(N)C3)cc2-c2ccc(C#N)c(F)c2)cc1 ; COc1ccc(-c2sc(C(=O)N3CCCN(CC(F)(F)F)CC3)cc2-c2ccc(C#N)c(F)c2)cc1F]

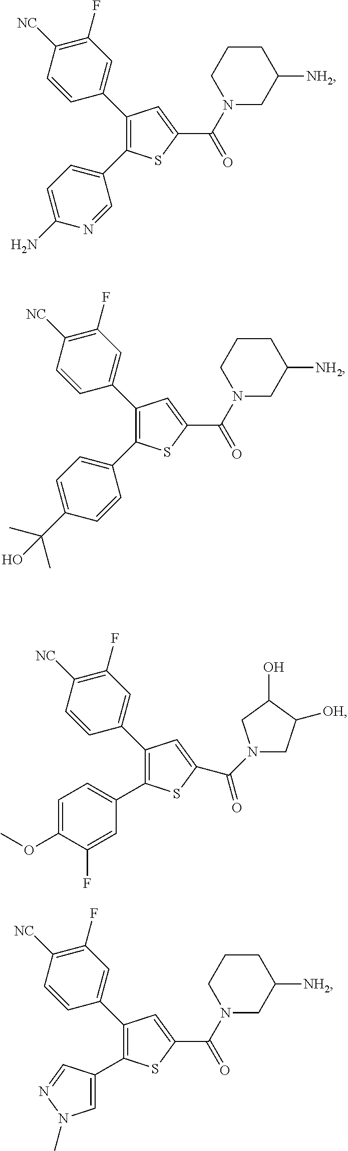

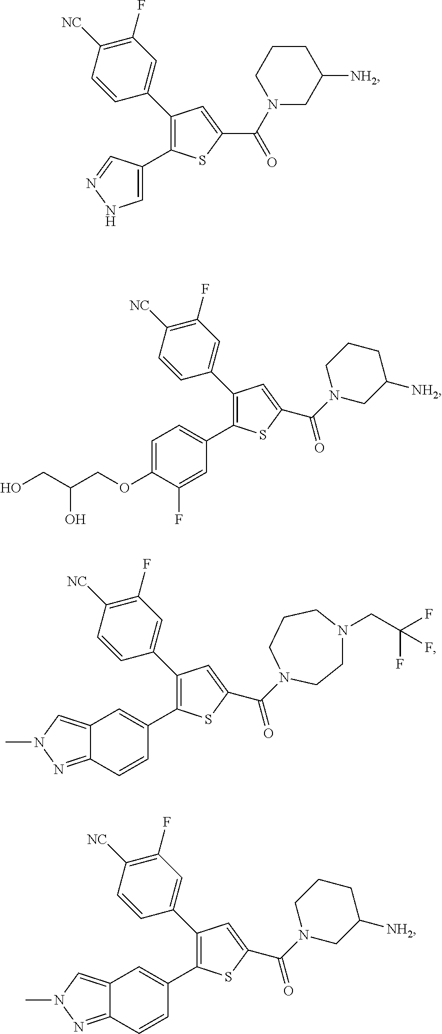 [SMILES: N#Cc1ccc(-c2cc(C(=O)N3CCCC(N)C3)sc2-c2cn[nH]c2)cc1F ; N#Cc1ccc(-c2cc(C(=O)N3CCCC(N)C3)sc2-c2ccc(OCC(O)CO)c(F)c2)cc1F ; Cn1cc2cc(-c3sc(C(=O)N4CCCN(CC(F)(F)F)CC4)cc3-c3ccc(C#N)c(F)c3)ccc2n1 ; Cn1cc2cc(-c3sc(C(=O)N4CCCC(N)C4)cc3-c3ccc(C#N)c(F)c3)ccc2n1]

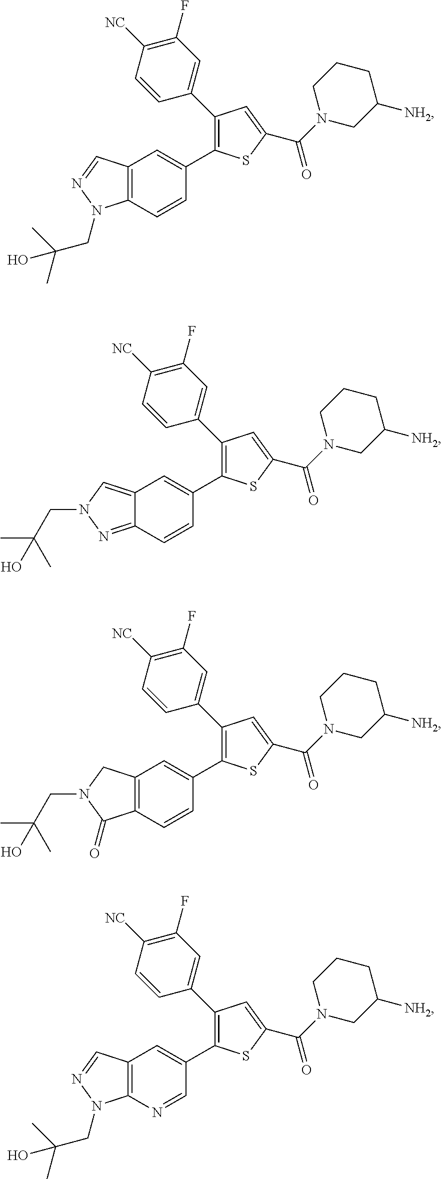 [SMILES: CC(C)(O)Cn1ncc2cc(-c3sc(C(=O)N4CCCC(N)C4)cc3-c3ccc(C#N)c(F)c3)ccc21 ; CC(C)(O)Cn1cc2cc(-c3sc(C(=O)N4CCCC(N)C4)cc3-c3ccc(C#N)c(F)c3)ccc2n1 ; CC(C)(O)CN1Cc2cc(-c3sc(C(=O)N4CCCC(N)C4)cc3-c3ccc(C#N)c(F)c3)ccc2C1=O ; CC(C)(O)Cn1ncc2cc(-c3sc(C(=O)N4CCCC(N)C4)cc3-c3ccc(C#N)c(F)c3)cnc21]

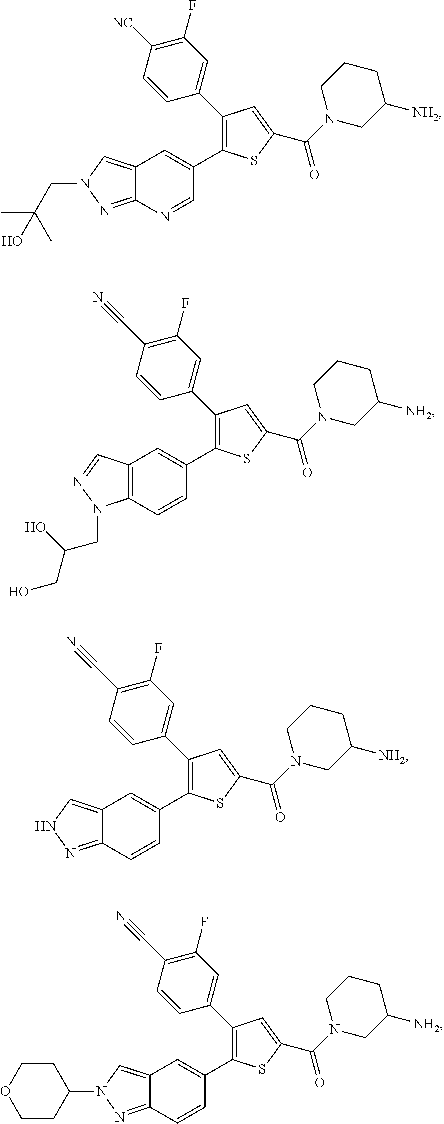 [SMILES: CC(C)(O)Cn1cc2cc(-c3sc(C(=O)N4CCCC(N)C4)cc3-c3ccc(C#N)c(F)c3)cnc2n1 ; N#Cc1ccc(-c2cc(C(=O)N3CCCC(N)C3)sc2-c2ccc3c(cnn3CC(O)CO)c2)cc1F ; N#Cc1ccc(-c2cc(C(=O)N3CCCC(N)C3)sc2-c2ccc3n[nH]cc3c2)cc1F ; N#Cc1ccc(-c2cc(C(=O)N3CCCC(N)C3)sc2-c2ccc3nn(C4CCOCC4)cc3c2)cc1F]

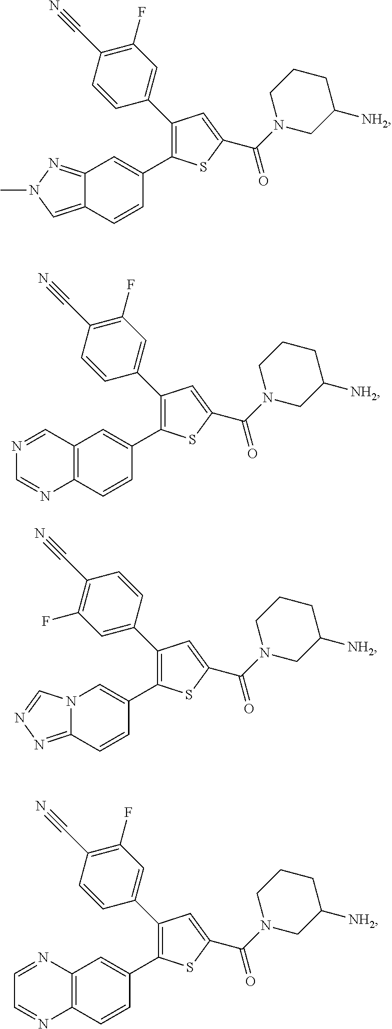

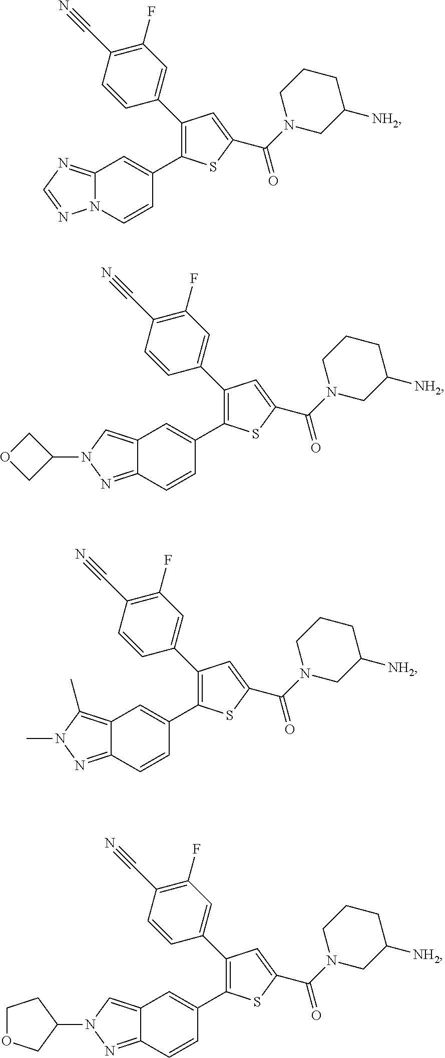 [SMILES: N#Cc1ccc(-c2cc(C(=O)N3CCCC(N)C3)sc2-c2ccn3ncnc3c2)cc1F ; N#Cc1ccc(-c2cc(C(=O)N3CCCC(N)C3)sc2-c2ccc3nn(C4COC4)cc3c2)cc1F ; Cc1c2cc(-c3sc(C(=O)N4CCCC(N)C4)cc3-c3ccc(C#N)c(F)c3)ccc2nn1C ; N#Cc1ccc(-c2cc(C(=O)N3CCCC(N)C3)sc2-c2ccc3nn(C4CCOC4)cc3c2)cc1F]

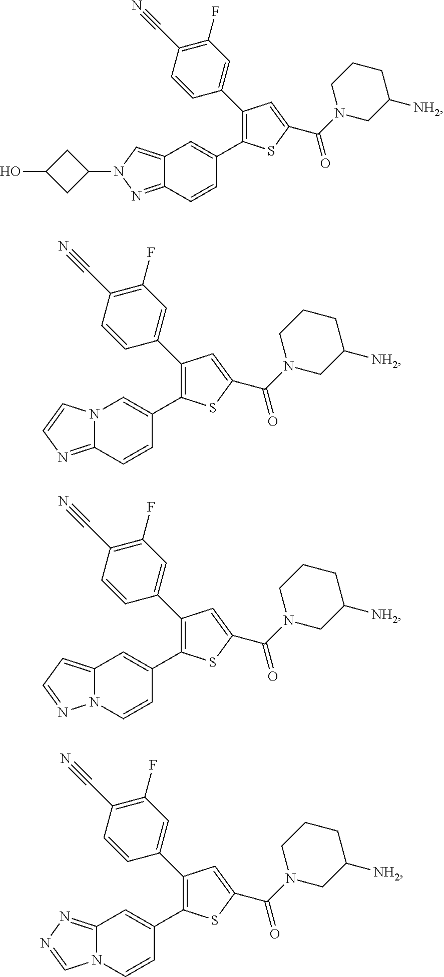

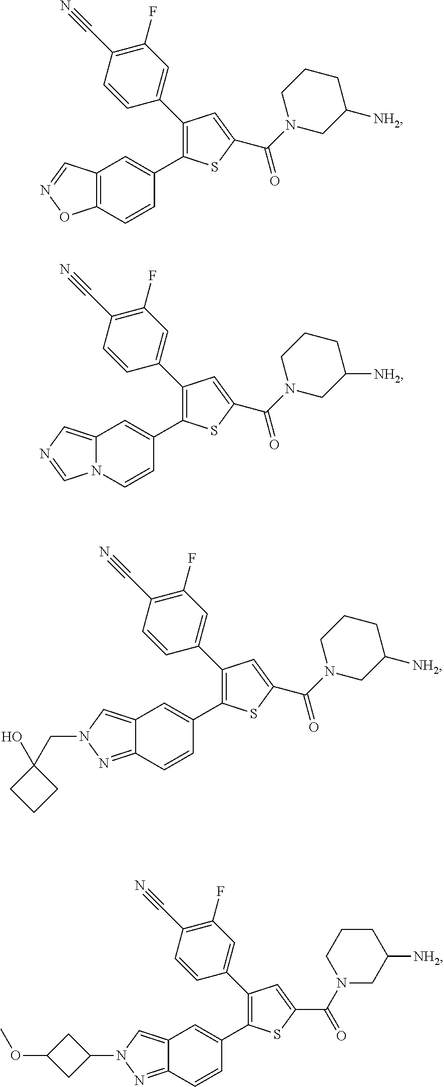 [SMILES: N#Cc1ccc(-c2cc(C(=O)N3CCCC(N)C3)sc2-c2ccc3oncc3c2)cc1F ; N#Cc1ccc(-c2cc(C(=O)N3CCCC(N)C3)sc2-c2ccn3cncc3c2)cc1F ; N#Cc1ccc(-c2cc(C(=O)N3CCCC(N)C3)sc2-c2ccc3nn(CC4(O)CCC4)cc3c2)cc1F ; COC1CC(n2cc3cc(-c4sc(C(=O)N5CCCC(N)C5)cc4-c4ccc(C#N)c(F)c4)ccc3n2)C1]

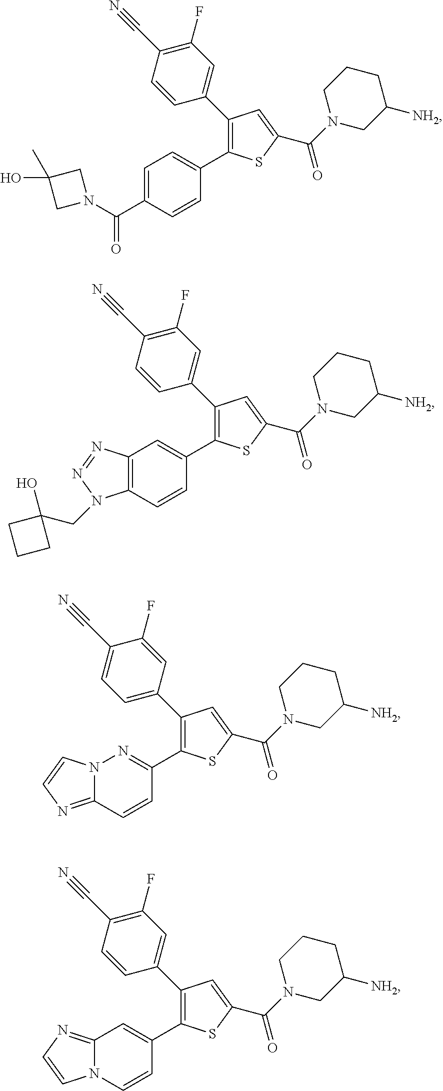

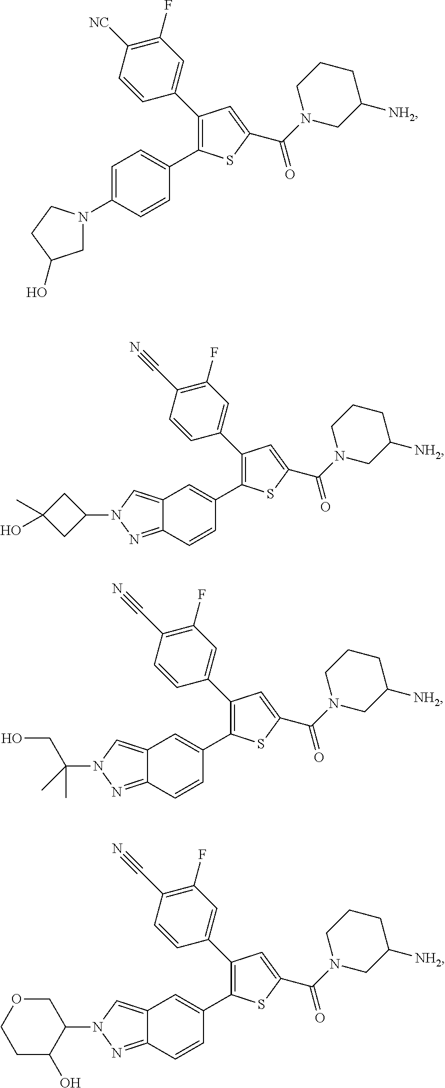 [SMILES: N#Cc1ccc(-c2cc(C(=O)N3CCCC(N)C3)sc2-c2ccc(N3CCC(O)C3)cc2)cc1F ; CC1(O)CC(n2cc3cc(-c4sc(C(=O)N5CCCC(N)C5)cc4-c4ccc(C#N)c(F)c4)ccc3n2)C1 ; CC(C)(CO)n1cc2cc(-c3sc(C(=O)N4CCCC(N)C4)cc3-c3ccc(C#N)c(F)c3)ccc2n1 ; N#Cc1ccc(-c2cc(C(=O)N3CCCC(N)C3)sc2-c2ccc3nn(C4COCCC4O)cc3c2)cc1F]

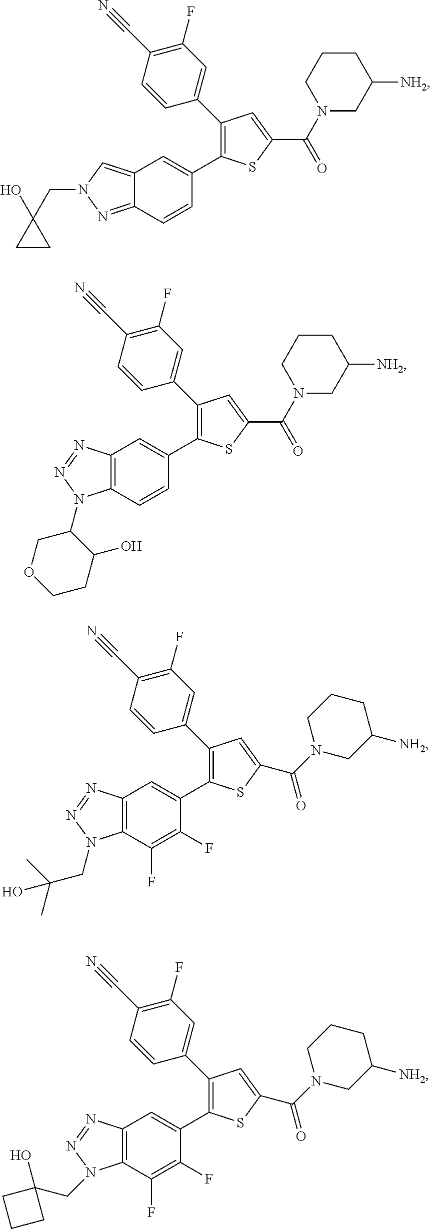

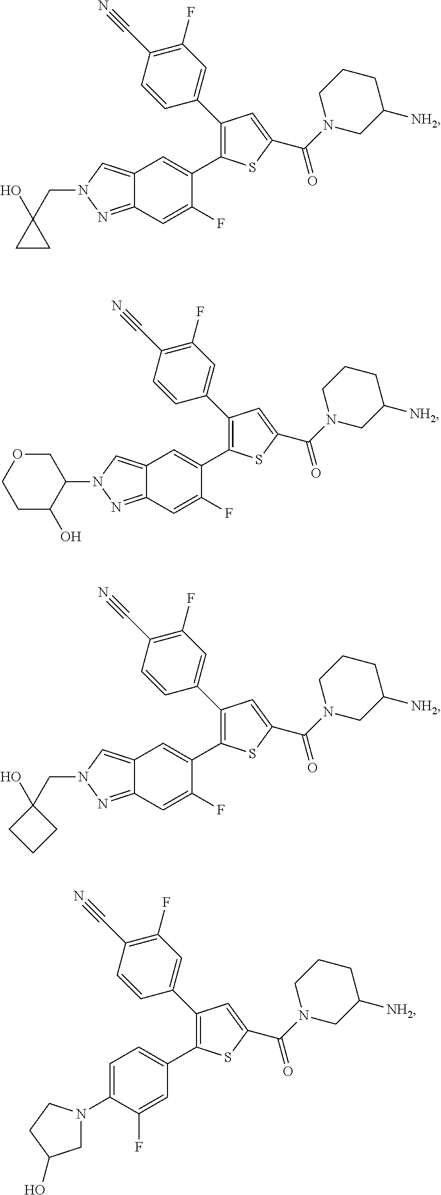 [SMILES: N#Cc1ccc(-c2cc(C(=O)N3CCCC(N)C3)sc2-c2cc3cn(CC4(O)CC4)nc3cc2F)cc1F ; N#Cc1ccc(-c2cc(C(=O)N3CCCC(N)C3)sc2-c2cc3cn(C4COCCC4O)nc3cc2F)cc1F ; N#Cc1ccc(-c2cc(C(=O)N3CCCC(N)C3)sc2-c2cc3cn(CC4(O)CCC4)nc3cc2F)cc1F ; N#Cc1ccc(-c2cc(C(=O)N3CCCC(N)C3)sc2-c2ccc(N3CCC(O)C3)c(F)c2)cc1F]

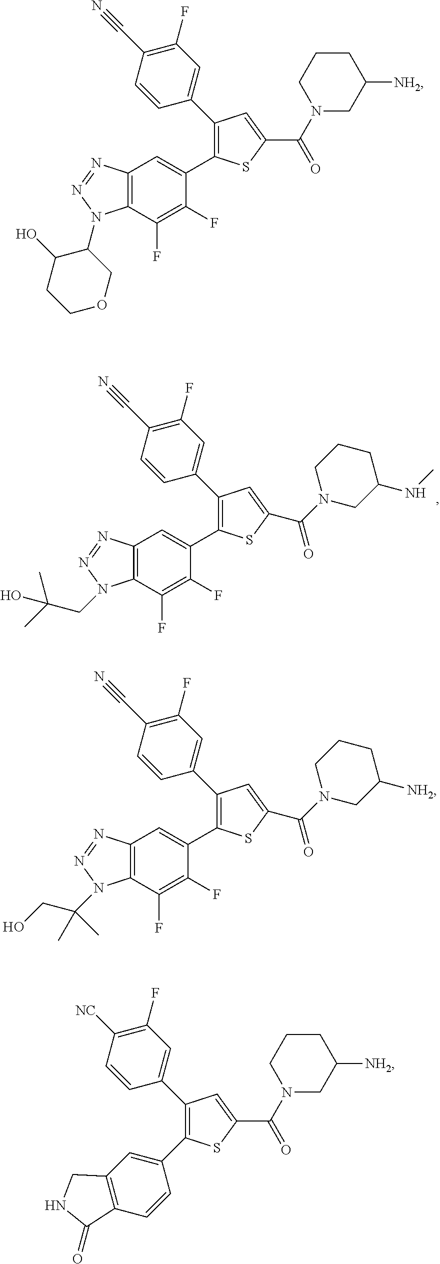 [SMILES: N#Cc1ccc(-c2cc(C(=O)N3CCCC(N)C3)sc2-c2cc3nnn(C4COCCC4O)c3c(F)c2F)cc1F ; CNC1CCCN(C(=O)c2cc(-c3ccc(C#N)c(F)c3)c(-c3cc4nnn(CC(C)(C)O)c4c(F)c3F)s2)C1 ; CC(C)(CO)n1nnc2cc(-c3sc(C(=O)N4CCCC(N)C4)cc3-c3ccc(C#N)c(F)c3)c(F)c(F)c21 ; N#Cc1ccc(-c2cc(C(=O)N3CCCC(N)C3)sc2-c2ccc3c(c2)CNC3=O)cc1F]

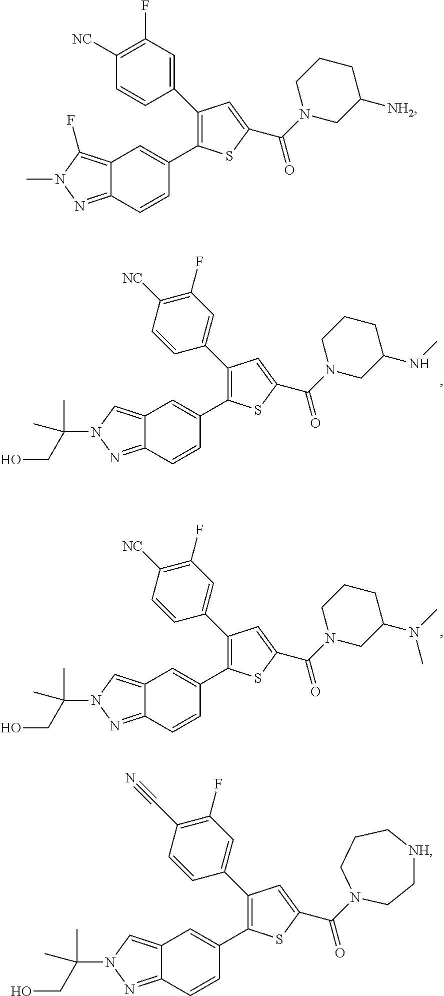 [SMILES: Cn1nc2ccc(-c3sc(C(=O)N4CCCC(N)C4)cc3-c3ccc(C#N)c(F)c3)cc2c1F ; CNC1CCCN(C(=O)c2cc(-c3ccc(C#N)c(F)c3)c(-c3ccc4nn(C(C)(C)CO)cc4c3)s2)C1 ; CN(C)C1CCCN(C(=O)c2cc(-c3ccc(C#N)c(F)c3)c(-c3ccc4nn(C(C)(C)CO)cc4c3)s2)C1 ; CC(C)(CO)n1cc2cc(-c3sc(C(=O)N4CCCNCC4)cc3-c3ccc(C#N)c(F)c3)ccc2n1]

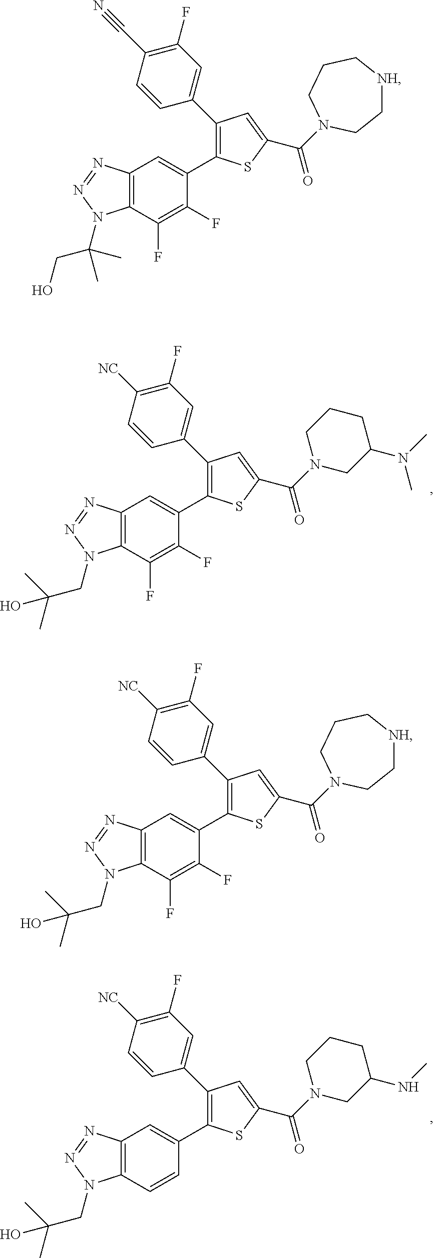 [SMILES: CC(C)(CO)n1nnc2cc(-c3sc(C(=O)N4CCCNCC4)cc3-c3ccc(C#N)c(F)c3)c(F)c(F)c21 ; CN(C)C1CCCN(C(=O)c2cc(-c3ccc(C#N)c(F)c3)c(-c3cc4nnn(CC(C)(C)O)c4c(F)c3F)s2)C1 ; CC(C)(O)Cn1nnc2cc(-c3sc(C(=O)N4CCCNCC4)cc3-c3ccc(C#N)c(F)c3)c(F)c(F)c21 ; CNC1CCCN(C(=O)c2cc(-c3ccc(C#N)c(F)c3)c(-c3ccc4c(c3)nnn4CC(C)(C)O)s2)C1]

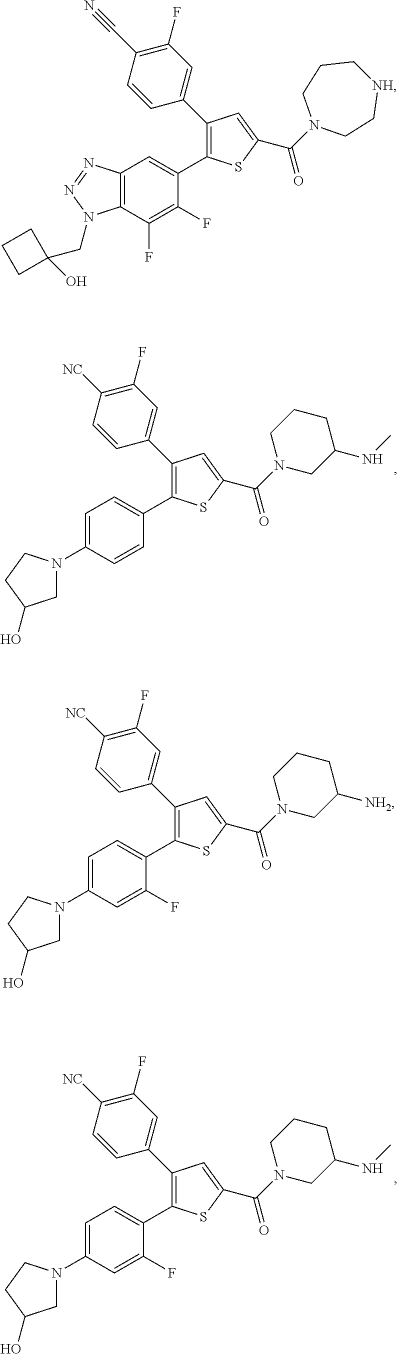 [SMILES: N#Cc1ccc(-c2cc(C(=O)N3CCCNCC3)sc2-c2cc3nnn(CC4(O)CCC4)c3c(F)c2F)cc1F ; CNC1CCCN(C(=O)c2cc(-c3ccc(C#N)c(F)c3)c(-c3ccc(N4CCC(O)C4)cc3)s2)C1 ; N#Cc1ccc(-c2cc(C(=O)N3CCCC(N)C3)sc2-c2ccc(N3CCC(O)C3)cc2F)cc1F ; CNC1CCCN(C(=O)c2cc(-c3ccc(C#N)c(F)c3)c(-c3ccc(N4CCC(O)C4)cc3F)s2)C1]

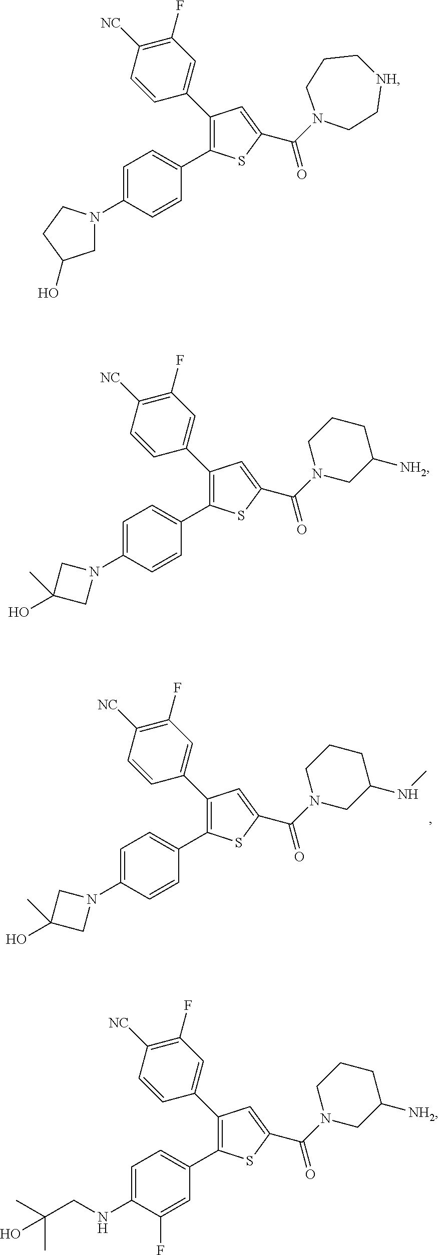

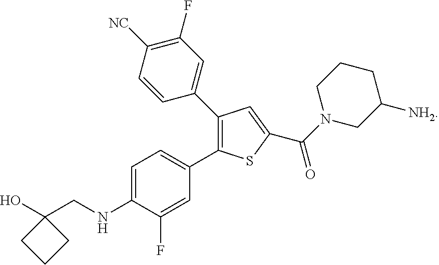 [SMILES: N#Cc1ccc(-c2cc(C(=O)N3CCCC(N)C3)sc2-c2ccc(NCC3(O)CCC3)c(F)c2)cc1F]

In some embodiments of the present disclosure, the compound or the pharmaceutically acceptable salt thereof is selected from:

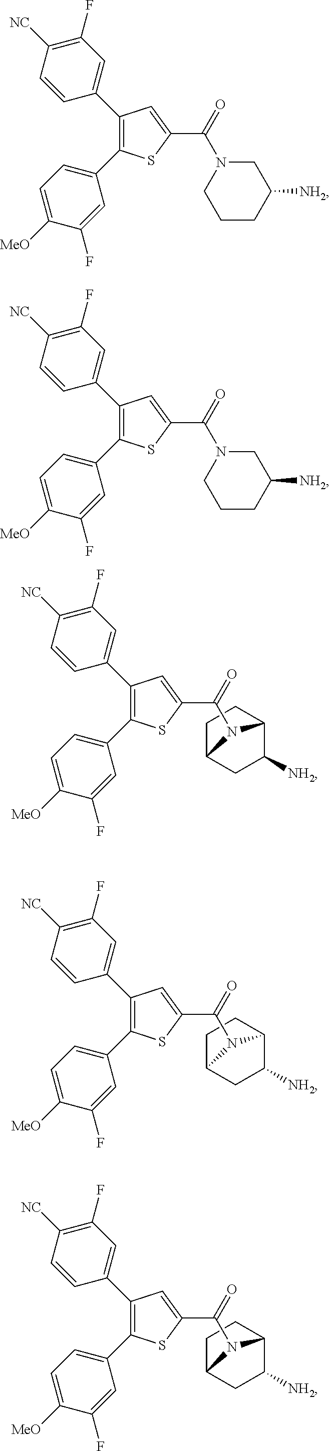

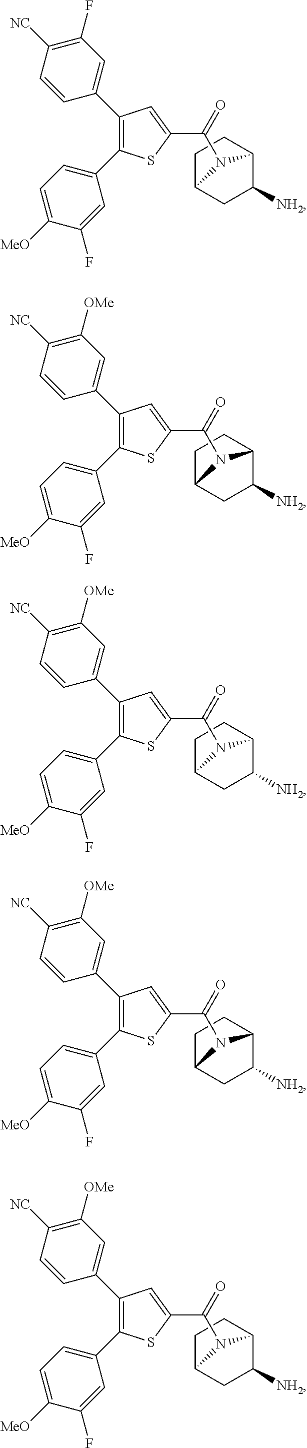

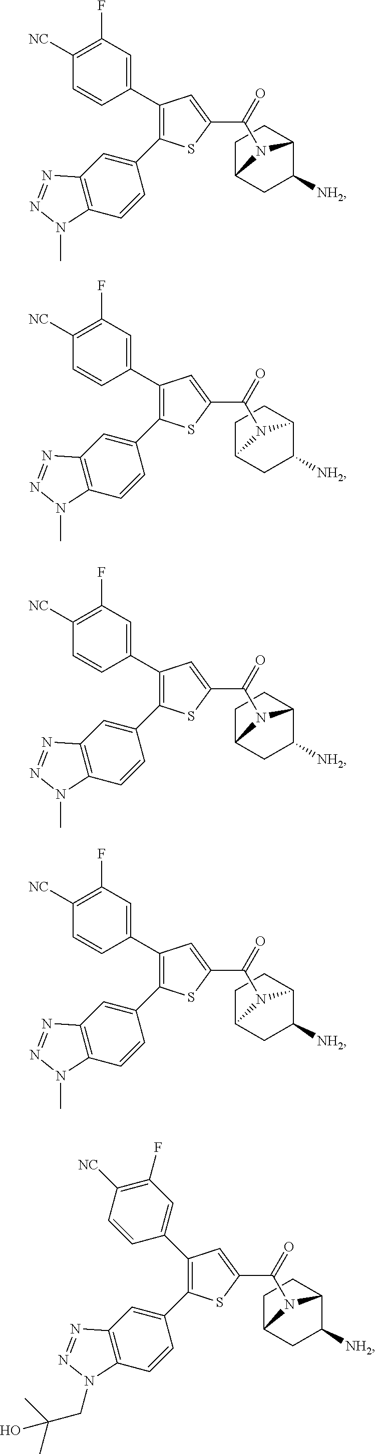 [SMILES: Cn1nnc2cc(-c3sc(C(=O)N4[C@H]5CC[C@@H]4[C@@H](N)C5)cc3-c3ccc(C#N)c(F)c3)ccc21 ; Cn1nnc2cc(-c3sc(C(=O)N4[C@@H]5CC[C@H]4[C@H](N)C5)cc3-c3ccc(C#N)c(F)c3)ccc21 ; Cn1nnc2cc(-c3sc(C(=O)N4[C@H]5CC[C@@H]4[C@H](N)C5)cc3-c3ccc(C#N)c(F)c3)ccc21 ; Cn1nnc2cc(-c3sc(C(=O)N4[C@@H]5CC[C@H]4[C@@H](N)C5)cc3-c3ccc(C#N)c(F)c3)ccc21 ; CC(C)(O)Cn1nnc2cc(-c3sc(C(=O)N4[C@H]5CC[C@@H]4[C@@H](N)C5)cc3-c3ccc(C#N)c(F)c3)ccc21]

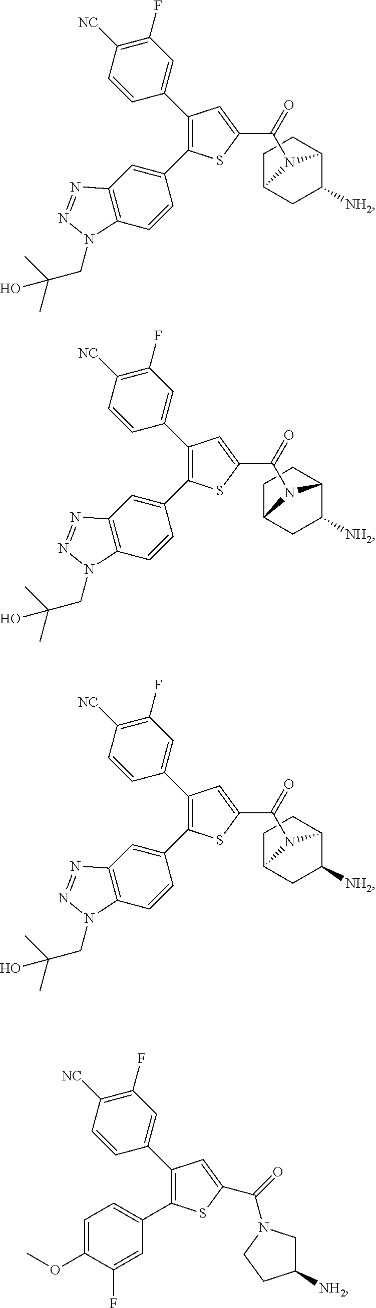 [SMILES: CC(C)(O)Cn1nnc2cc(-c3sc(C(=O)N4[C@@H]5CC[C@H]4[C@H](N)C5)cc3-c3ccc(C#N)c(F)c3)ccc21 ; CC(C)(O)Cn1nnc2cc(-c3sc(C(=O)N4[C@H]5CC[C@@H]4[C@H](N)C5)cc3-c3ccc(C#N)c(F)c3)ccc21 ; CC(C)(O)Cn1nnc2cc(-c3sc(C(=O)N4[C@@H]5CC[C@H]4[C@@H](N)C5)cc3-c3ccc(C#N)c(F)c3)ccc21 ; COc1ccc(-c2sc(C(=O)N3CC[C@H](N)C3)cc2-c2ccc(C#N)c(F)c2)cc1F]

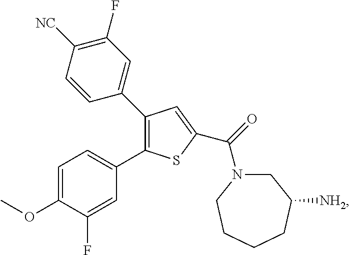 [SMILES: COc1ccc(-c2sc(C(=O)N3CCCC[C@@H](N)C3)cc2-c2ccc(C#N)c(F)c2)cc1F]

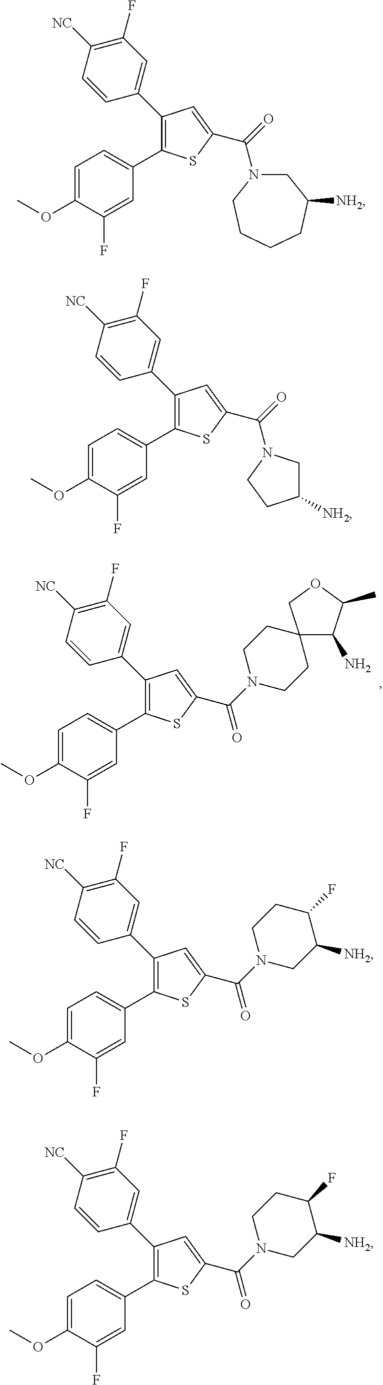

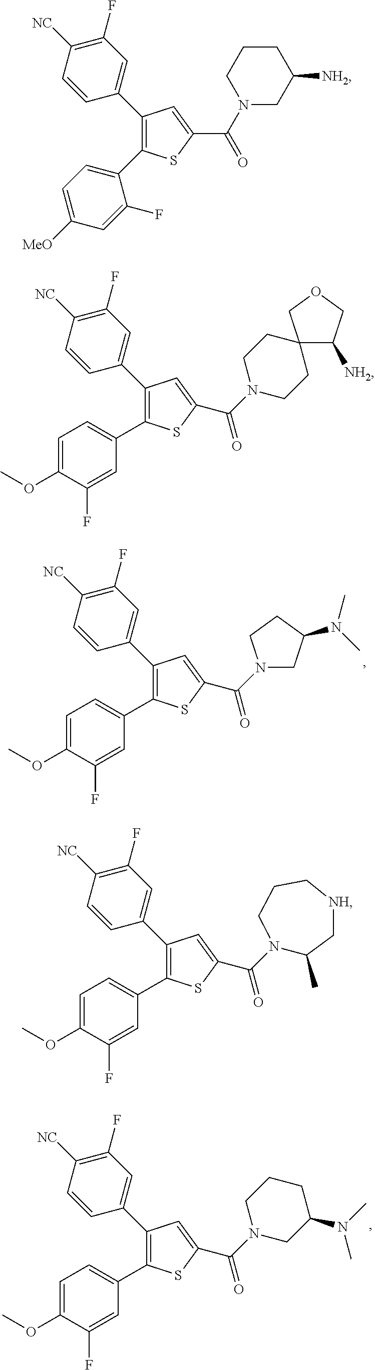

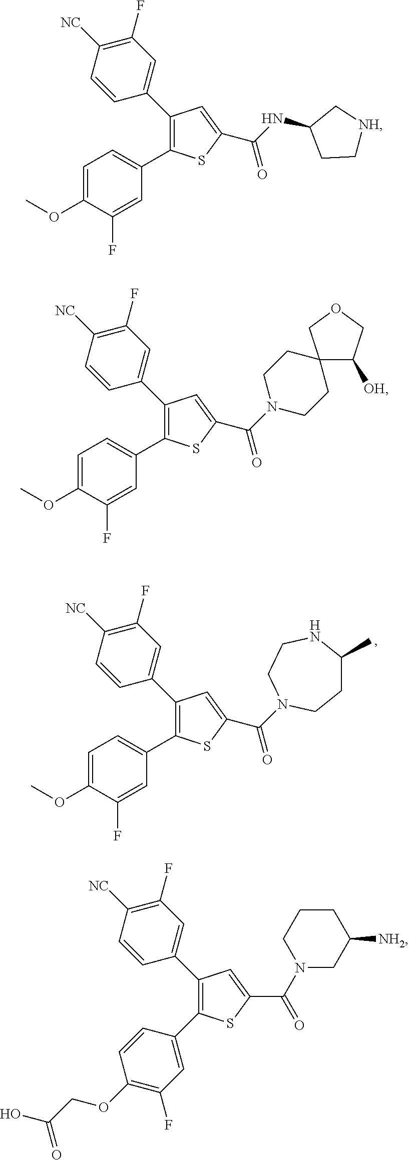

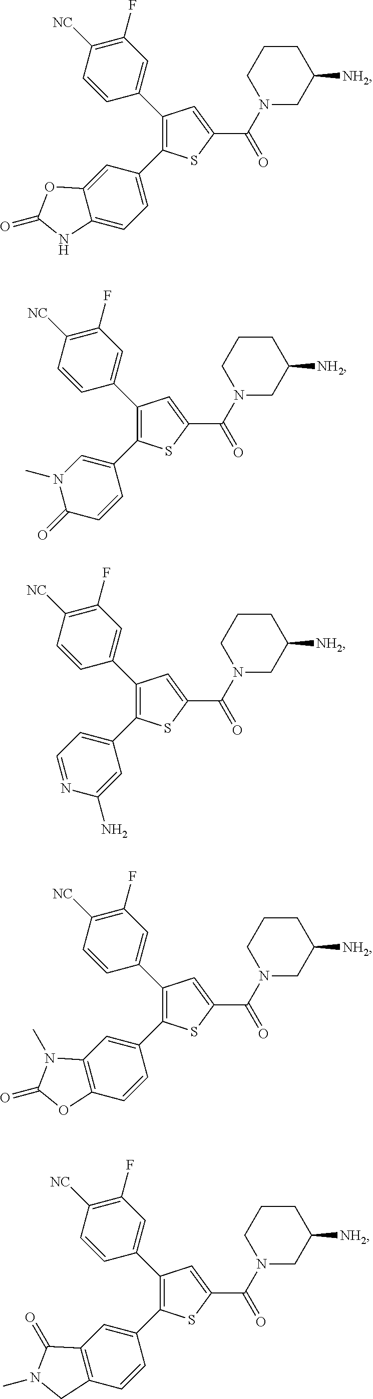 [SMILES: N#Cc1ccc(-c2cc(C(=O)N3CCC[C@@H](N)C3)sc2-c2ccc3[nH]c(=O)oc3c2)cc1F ; Cn1cc(-c2sc(C(=O)N3CCC[C@@H](N)C3)cc2-c2ccc(C#N)c(F)c2)ccc1=O ; N#Cc1ccc(-c2cc(C(=O)N3CCC[C@@H](N)C3)sc2-c2ccnc(N)c2)cc1F ; Cn1c(=O)oc2ccc(-c3sc(C(=O)N4CCC[C@@H](N)C4)cc3-c3ccc(C#N)c(F)c3)cc21 ; CN1Cc2ccc(-c3sc(C(=O)N4CCC[C@@H](N)C4)cc3-c3ccc(C#N)c(F)c3)cc2C1=O]

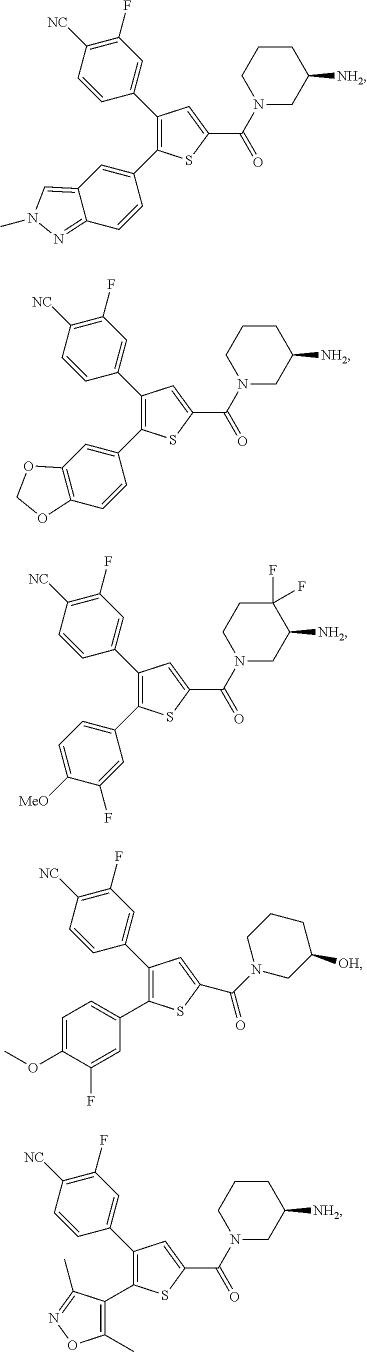

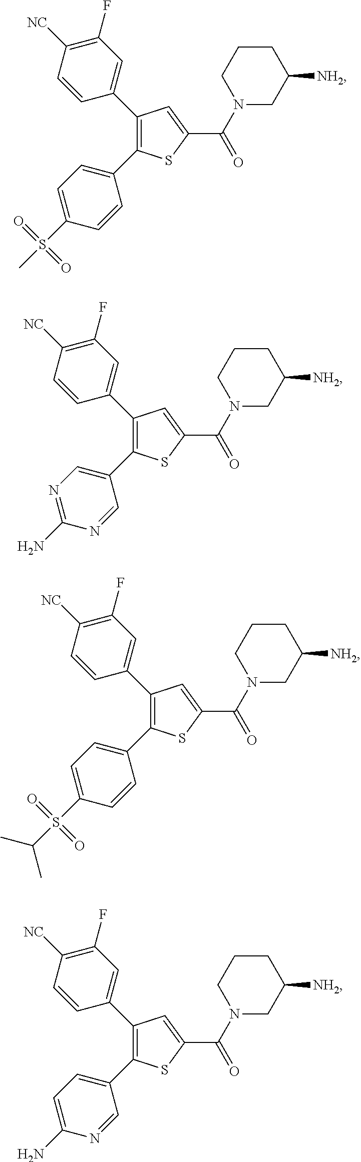 [SMILES: CS(=O)(=O)c1ccc(-c2sc(C(=O)N3CCC[C@@H](N)C3)cc2-c2ccc(C#N)c(F)c2)cc1 ; N#Cc1ccc(-c2cc(C(=O)N3CCC[C@@H](N)C3)sc2-c2cnc(N)nc2)cc1F ; CC(C)S(=O)(=O)c1ccc(-c2sc(C(=O)N3CCC[C@@H](N)C3)cc2-c2ccc(C#N)c(F)c2)cc1 ; N#Cc1ccc(-c2cc(C(=O)N3CCC[C@@H](N)C3)sc2-c2ccc(N)nc2)cc1F]

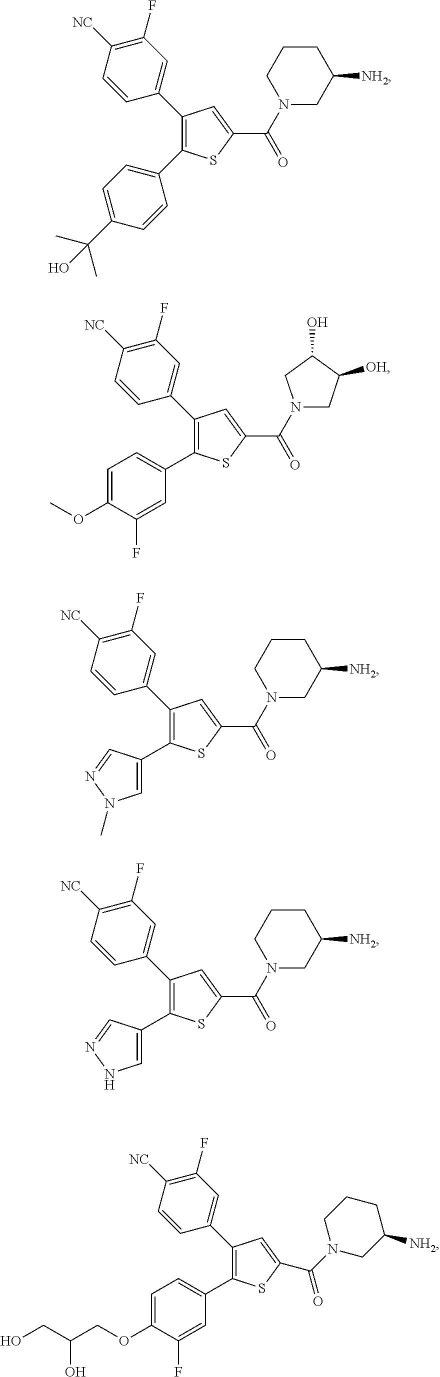

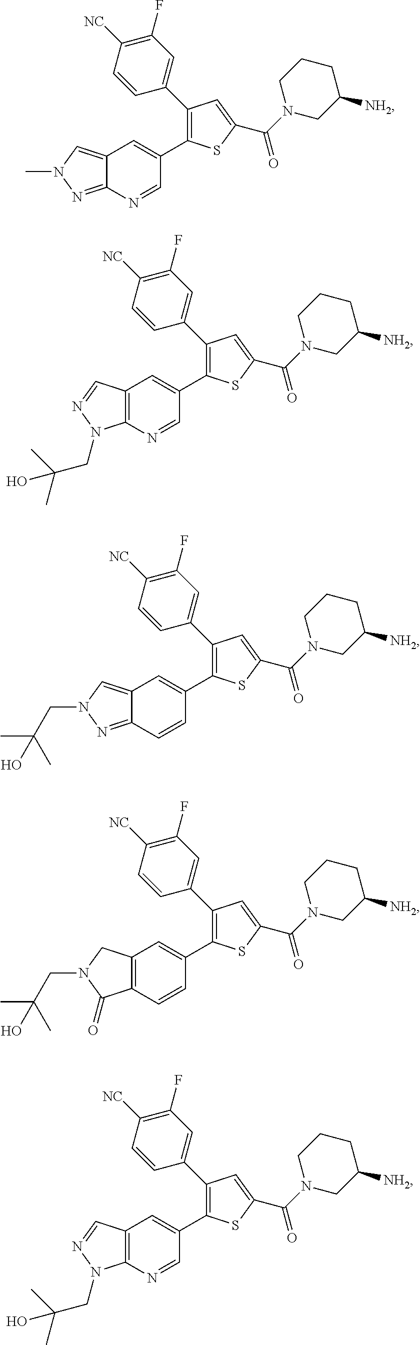 [SMILES: Cn1cc2cc(-c3sc(C(=O)N4CCC[C@@H](N)C4)cc3-c3ccc(C#N)c(F)c3)cnc2n1 ; CC(C)(O)Cn1ncc2cc(-c3sc(C(=O)N4CCC[C@@H](N)C4)cc3-c3ccc(C#N)c(F)c3)cnc21 ; CC(C)(O)Cn1cc2cc(-c3sc(C(=O)N4CCC[C@@H](N)C4)cc3-c3ccc(C#N)c(F)c3)ccc2n1 ; CC(C)(O)CN1Cc2cc(-c3sc(C(=O)N4CCC[C@@H](N)C4)cc3-c3ccc(C#N)c(F)c3)ccc2C1=O ; CC(C)(O)Cn1ncc2cc(-c3sc(C(=O)N4CCC[C@@H](N)C4)cc3-c3ccc(C#N)c(F)c3)cnc21]

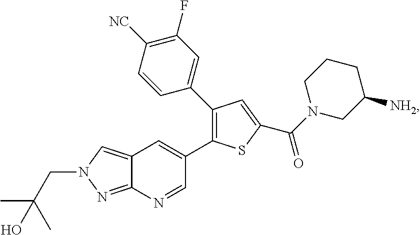 [SMILES: CC(C)(O)Cn1cc2cc(-c3sc(C(=O)N4CCC[C@@H](N)C4)cc3-c3ccc(C#N)c(F)c3)cnc2n1]

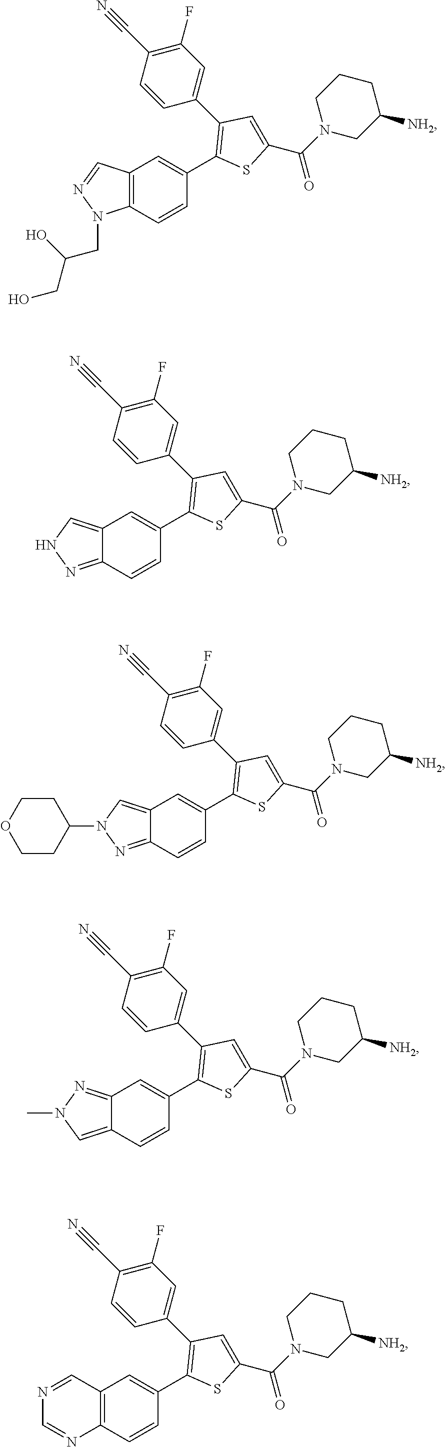 [SMILES: N#Cc1ccc(-c2cc(C(=O)N3CCC[C@@H](N)C3)sc2-c2ccc3c(cnn3CC(O)CO)c2)cc1F ; N#Cc1ccc(-c2cc(C(=O)N3CCC[C@@H](N)C3)sc2-c2ccc3n[nH]cc3c2)cc1F ; N#Cc1ccc(-c2cc(C(=O)N3CCC[C@@H](N)C3)sc2-c2ccc3nn(C4CCOCC4)cc3c2)cc1F ; Cn1cc2ccc(-c3sc(C(=O)N4CCC[C@@H](N)C4)cc3-c3ccc(C#N)c(F)c3)cc2n1 ; N#Cc1ccc(-c2cc(C(=O)N3CCC[C@@H](N)C3)sc2-c2ccc3ncncc3c2)cc1F]

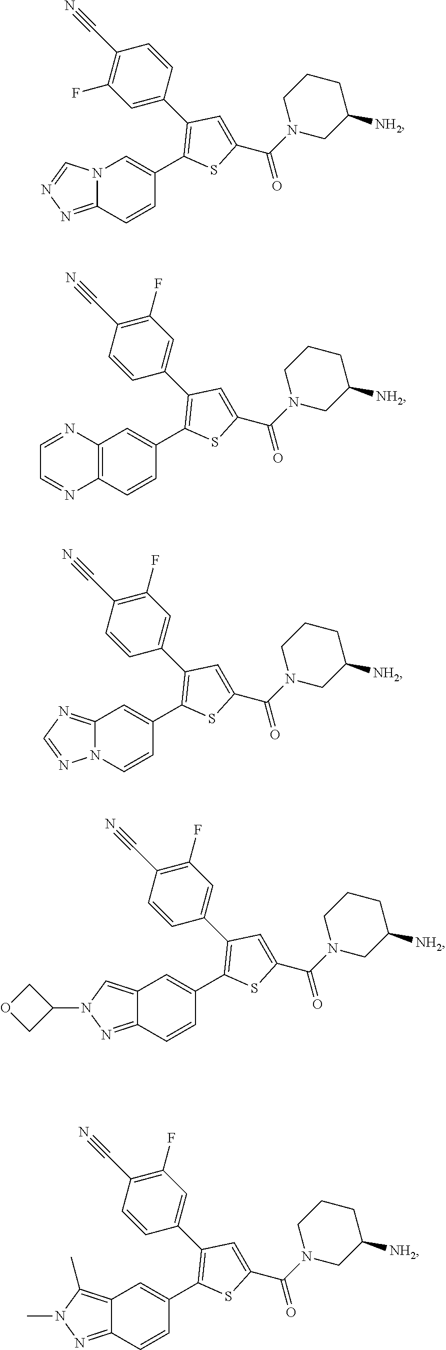 [SMILES: N#Cc1ccc(-c2cc(C(=O)N3CCC[C@@H](N)C3)sc2-c2ccc3nncn3c2)cc1F ; N#Cc1ccc(-c2cc(C(=O)N3CCC[C@@H](N)C3)sc2-c2ccc3nccnc3c2)cc1F ; N#Cc1ccc(-c2cc(C(=O)N3CCC[C@@H](N)C3)sc2-c2ccn3ncnc3c2)cc1F ; N#Cc1ccc(-c2cc(C(=O)N3CCC[C@@H](N)C3)sc2-c2ccc3nn(C4COC4)cc3c2)cc1F ; Cc1c2cc(-c3sc(C(=O)N4CCC[C@@H](N)C4)cc3-c3ccc(C#N)c(F)c3)ccc2nn1C]

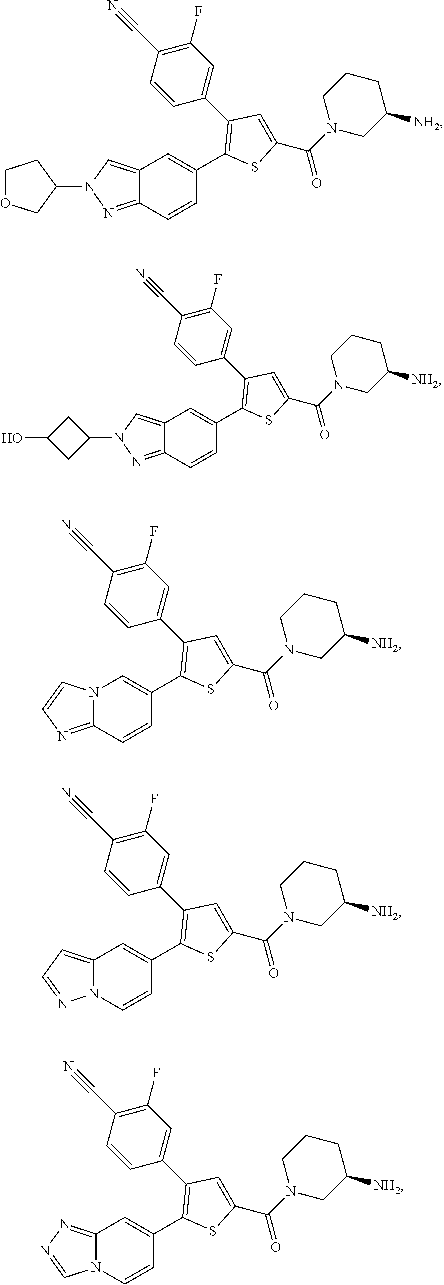 [SMILES: N#Cc1ccc(-c2cc(C(=O)N3CCC[C@@H](N)C3)sc2-c2ccc3nn(C4CCOC4)cc3c2)cc1F ; N#Cc1ccc(-c2cc(C(=O)N3CCC[C@@H](N)C3)sc2-c2ccc3nn(C4CC(O)C4)cc3c2)cc1F ; N#Cc1ccc(-c2cc(C(=O)N3CCC[C@@H](N)C3)sc2-c2ccc3nccn3c2)cc1F ; N#Cc1ccc(-c2cc(C(=O)N3CCC[C@@H](N)C3)sc2-c2ccn3nccc3c2)cc1F ; N#Cc1ccc(-c2cc(C(=O)N3CCC[C@@H](N)C3)sc2-c2ccn3cnnc3c2)cc1F]

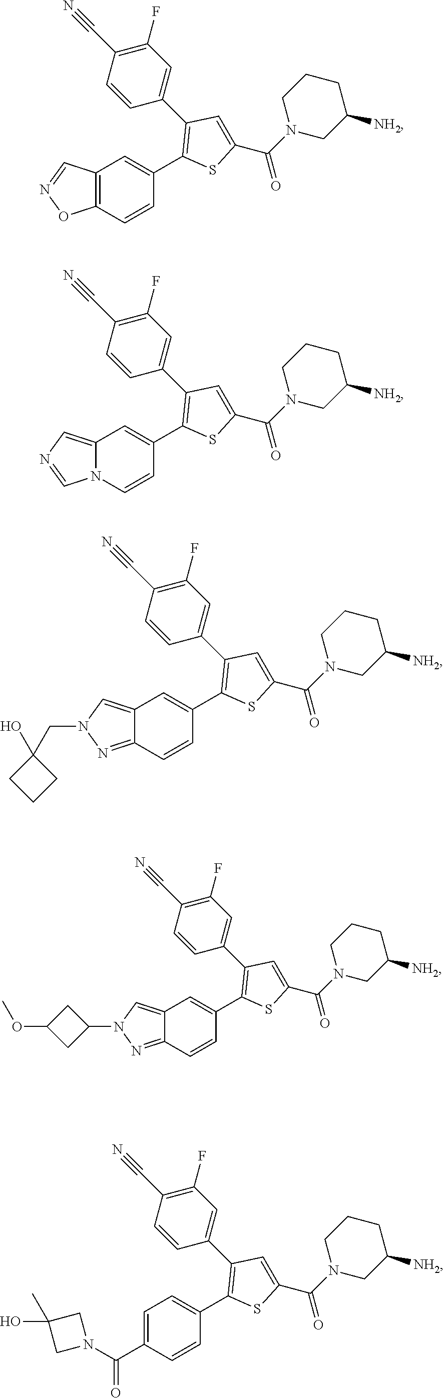 [SMILES: N#Cc1ccc(-c2cc(C(=O)N3CCC[C@@H](N)C3)sc2-c2ccc3oncc3c2)cc1F ; N#Cc1ccc(-c2cc(C(=O)N3CCC[C@@H](N)C3)sc2-c2ccn3cncc3c2)cc1F ; N#Cc1ccc(-c2cc(C(=O)N3CCC[C@@H](N)C3)sc2-c2ccc3nn(CC4(O)CCC4)cc3c2)cc1F ; COC1CC(n2cc3cc(-c4sc(C(=O)N5CCC[C@@H](N)C5)cc4-c4ccc(C#N)c(F)c4)ccc3n2)C1 ; CC1(O)CN(C(=O)c2ccc(-c3sc(C(=O)N4CCC[C@@H](N)C4)cc3-c3ccc(C#N)c(F)c3)cc2)C1]

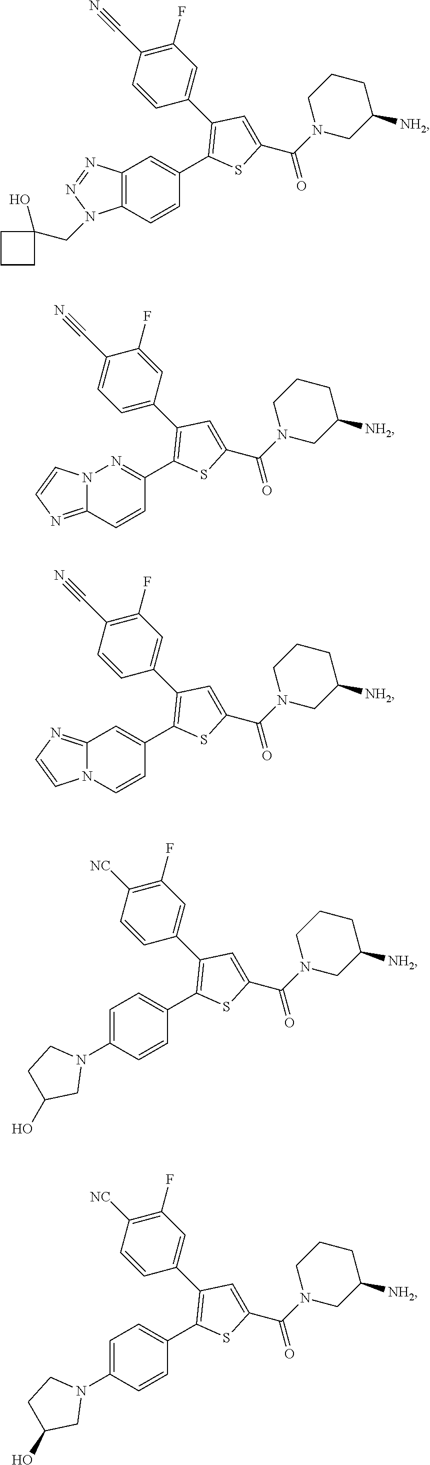 [SMILES: N#Cc1ccc(-c2cc(C(=O)N3CCC[C@@H](N)C3)sc2-c2ccc3c(c2)nnn3CC2(O)CCC2)cc1F ; N#Cc1ccc(-c2cc(C(=O)N3CCC[C@@H](N)C3)sc2-c2ccc3nccn3n2)cc1F ; N#Cc1ccc(-c2cc(C(=O)N3CCC[C@@H](N)C3)sc2-c2ccn3ccnc3c2)cc1F ; N#Cc1ccc(-c2cc(C(=O)N3CCC[C@@H](N)C3)sc2-c2ccc(N3CCC(O)C3)cc2)cc1F ; N#Cc1ccc(-c2cc(C(=O)N3CCC[C@@H](N)C3)sc2-c2ccc(N3CC[C@H](O)C3)cc2)cc1F]

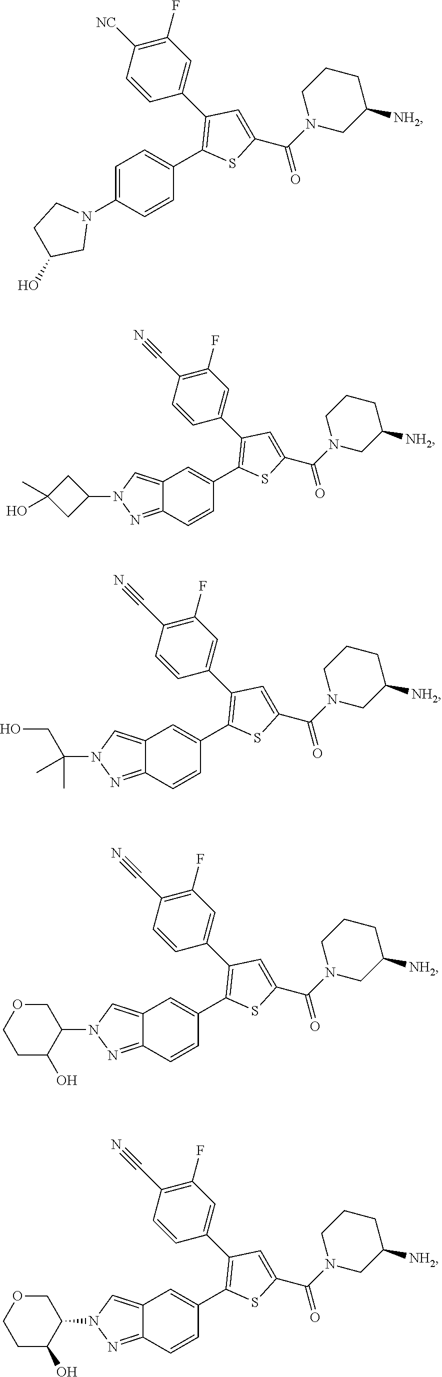 [SMILES: N#Cc1ccc(-c2cc(C(=O)N3CCC[C@@H](N)C3)sc2-c2ccc(N3CC[C@@H](O)C3)cc2)cc1F ; CC1(O)CC(n2cc3cc(-c4sc(C(=O)N5CCC[C@@H](N)C5)cc4-c4ccc(C#N)c(F)c4)ccc3n2)C1 ; CC(C)(CO)n1cc2cc(-c3sc(C(=O)N4CCC[C@@H](N)C4)cc3-c3ccc(C#N)c(F)c3)ccc2n1 ; N#Cc1ccc(-c2cc(C(=O)N3CCC[C@@H](N)C3)sc2-c2ccc3nn(C4COCCC4O)cc3c2)cc1F ; N#Cc1ccc(-c2cc(C(=O)N3CCC[C@@H](N)C3)sc2-c2ccc3nn([C@H]4COCC[C@@H]4O)cc3c2)cc1F]

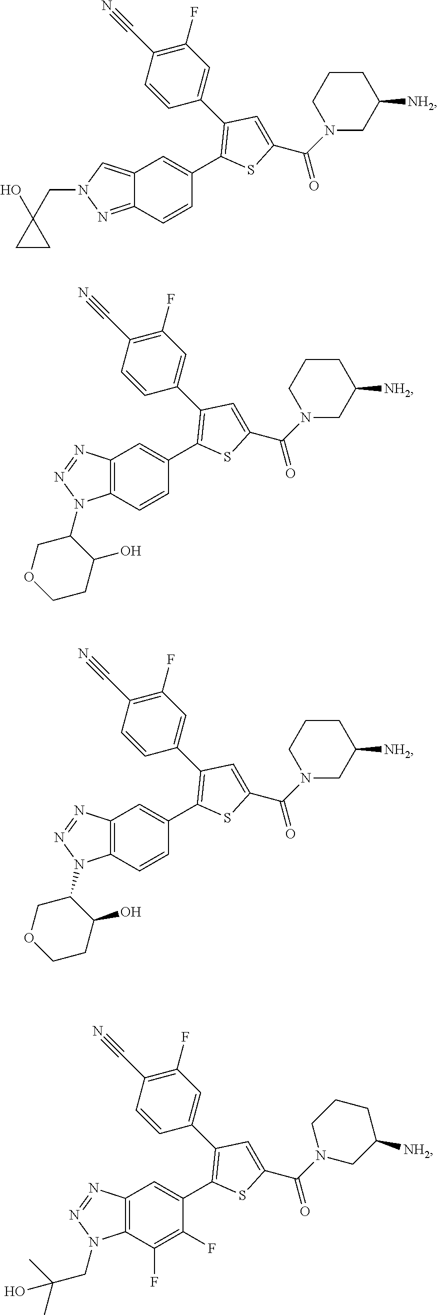 [SMILES: N#Cc1ccc(-c2cc(C(=O)N3CCC[C@@H](N)C3)sc2-c2ccc3nn(CC4(O)CC4)cc3c2)cc1F ; N#Cc1ccc(-c2cc(C(=O)N3CCC[C@@H](N)C3)sc2-c2ccc3c(c2)nnn3C2COCCC2O)cc1F ; N#Cc1ccc(-c2cc(C(=O)N3CCC[C@@H](N)C3)sc2-c2ccc3c(c2)nnn3[C@H]2COCC[C@@H]2O)cc1F ; CC(C)(O)Cn1nnc2cc(-c3sc(C(=O)N4CCC[C@@H](N)C4)cc3-c3ccc(C#N)c(F)c3)c(F)c(F)c21]

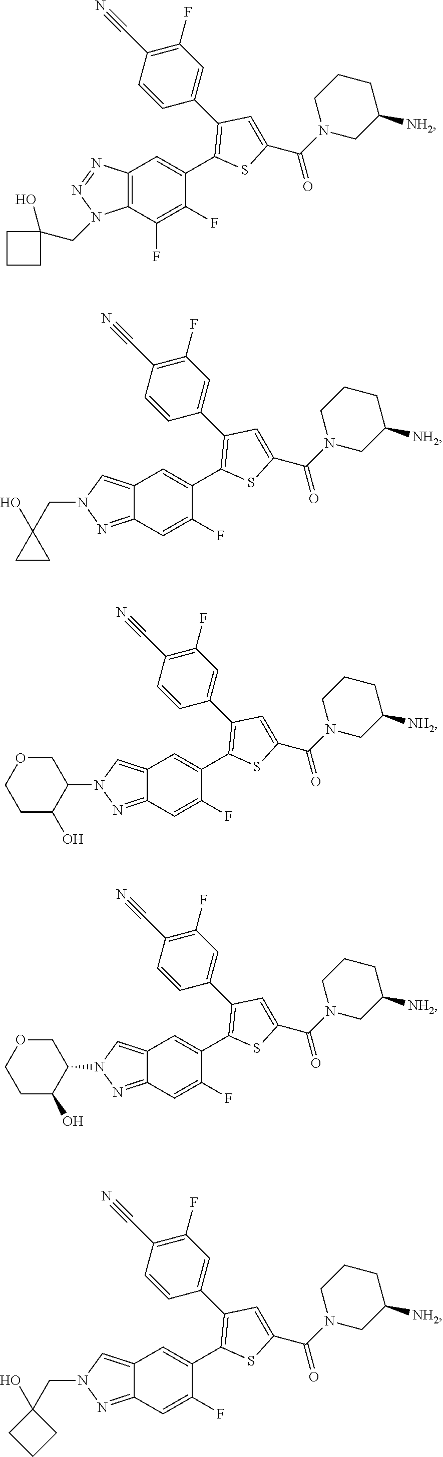 [SMILES: N#Cc1ccc(-c2cc(C(=O)N3CCC[C@@H](N)C3)sc2-c2cc3nnn(CC4(O)CCC4)c3c(F)c2F)cc1F ; N#Cc1ccc(-c2cc(C(=O)N3CCC[C@@H](N)C3)sc2-c2cc3cn(CC4(O)CC4)nc3cc2F)cc1F ; N#Cc1ccc(-c2cc(C(=O)N3CCC[C@@H](N)C3)sc2-c2cc3cn(C4COCCC4O)nc3cc2F)cc1F ; N#Cc1ccc(-c2cc(C(=O)N3CCC[C@@H](N)C3)sc2-c2cc3cn([C@H]4COCC[C@@H]4O)nc3cc2F)cc1F ; N#Cc1ccc(-c2cc(C(=O)N3CCC[C@@H](N)C3)sc2-c2cc3cn(CC4(O)CCC4)nc3cc2F)cc1F]

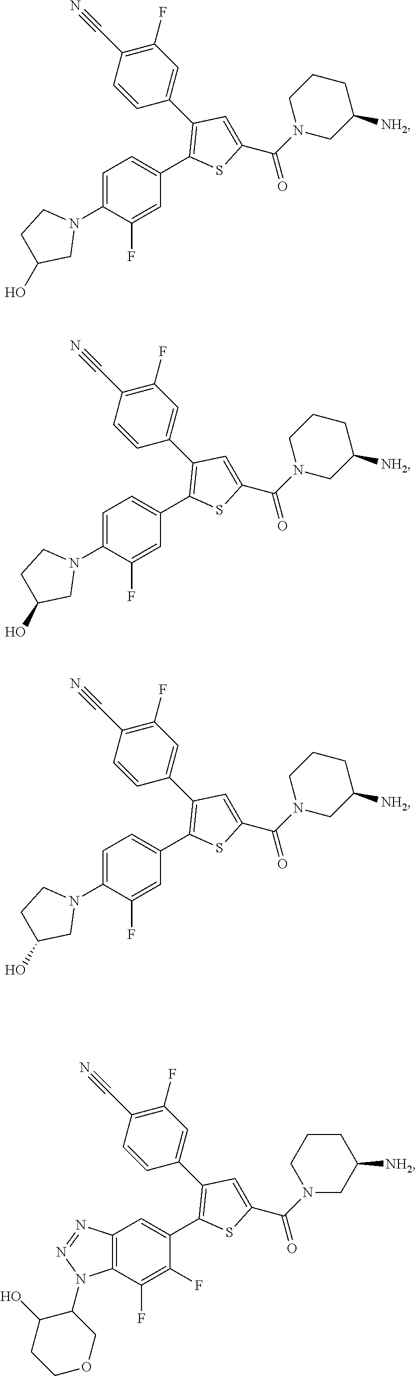 [SMILES: N#Cc1ccc(-c2cc(C(=O)N3CCC[C@@H](N)C3)sc2-c2ccc(N3CCC(O)C3)c(F)c2)cc1F ; N#Cc1ccc(-c2cc(C(=O)N3CCC[C@@H](N)C3)sc2-c2ccc(N3CC[C@H](O)C3)c(F)c2)cc1F ; N#Cc1ccc(-c2cc(C(=O)N3CCC[C@@H](N)C3)sc2-c2ccc(N3CC[C@@H](O)C3)c(F)c2)cc1F ; N#Cc1ccc(-c2cc(C(=O)N3CCC[C@@H](N)C3)sc2-c2cc3nnn(C4COCCC4O)c3c(F)c2F)cc1F]

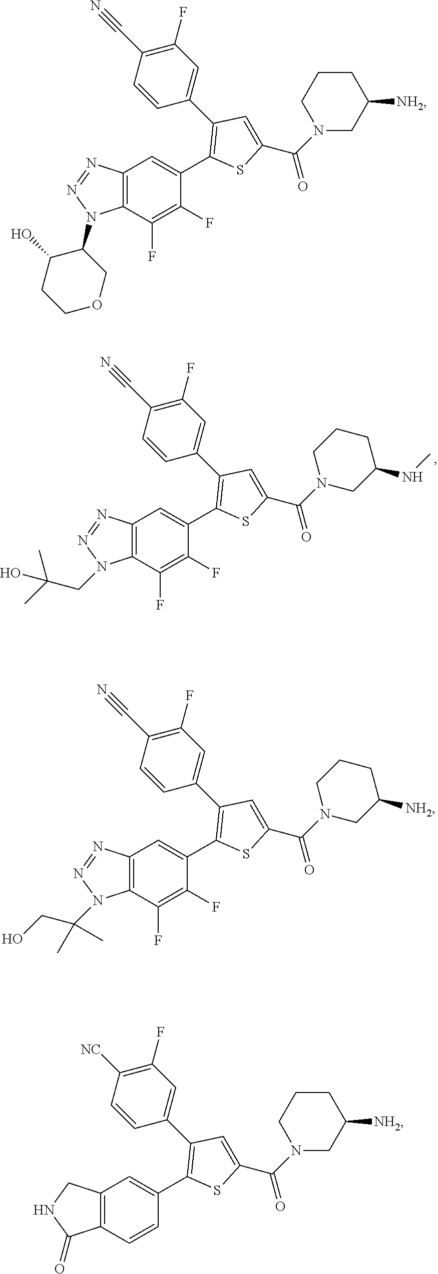 [SMILES: N#Cc1ccc(-c2cc(C(=O)N3CCC[C@@H](N)C3)sc2-c2cc3nnn([C@H]4COCC[C@@H]4O)c3c(F)c2F)cc1F ; CN[C@@H]1CCCN(C(=O)c2cc(-c3ccc(C#N)c(F)c3)c(-c3cc4nnn(CC(C)(C)O)c4c(F)c3F)s2)C1 ; CC(C)(CO)n1nnc2cc(-c3sc(C(=O)N4CCC[C@@H](N)C4)cc3-c3ccc(C#N)c(F)c3)c(F)c(F)c21 ; N#Cc1ccc(-c2cc(C(=O)N3CCC[C@@H](N)C3)sc2-c2ccc3c(c2)CNC3=O)cc1F]

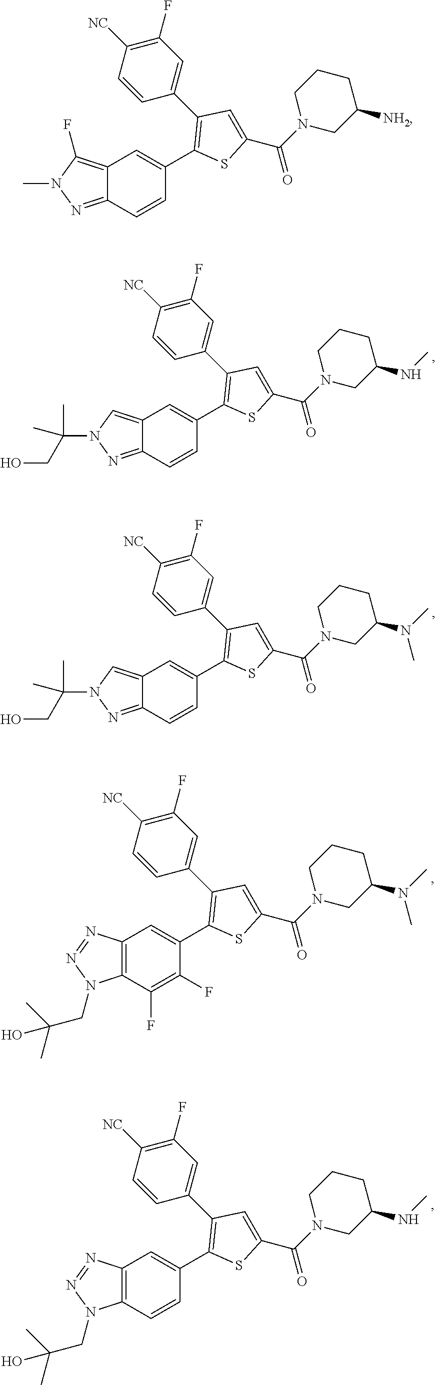 [SMILES: Cn1nc2ccc(-c3sc(C(=O)N4CCC[C@@H](N)C4)cc3-c3ccc(C#N)c(F)c3)cc2c1F ; CN[C@@H]1CCCN(C(=O)c2cc(-c3ccc(C#N)c(F)c3)c(-c3ccc4nn(C(C)(C)CO)cc4c3)s2)C1 ; CN(C)[C@@H]1CCCN(C(=O)c2cc(-c3ccc(C#N)c(F)c3)c(-c3ccc4nn(C(C)(C)CO)cc4c3)s2)C1 ; CN(C)[C@@H]1CCCN(C(=O)c2cc(-c3ccc(C#N)c(F)c3)c(-c3cc4nnn(CC(C)(C)O)c4c(F)c3F)s2)C1 ; CN[C@@H]1CCCN(C(=O)c2cc(-c3ccc(C#N)c(F)c3)c(-c3ccc4c(c3)nnn4CC(C)(C)O)s2)C1]

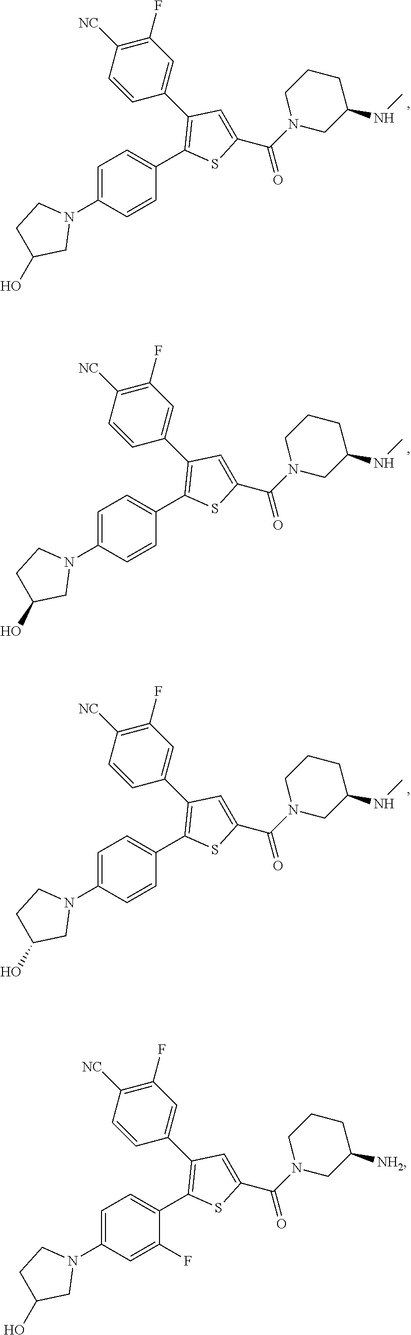 [SMILES: CN[C@@H]1CCCN(C(=O)c2cc(-c3ccc(C#N)c(F)c3)c(-c3ccc(N4CCC(O)C4)cc3)s2)C1 ; CN[C@@H]1CCCN(C(=O)c2cc(-c3ccc(C#N)c(F)c3)c(-c3ccc(N4CC[C@H](O)C4)cc3)s2)C1 ; CN[C@@H]1CCCN(C(=O)c2cc(-c3ccc(C#N)c(F)c3)c(-c3ccc(N4CC[C@@H](O)C4)cc3)s2)C1 ; N#Cc1ccc(-c2cc(C(=O)N3CCC[C@@H](N)C3)sc2-c2ccc(N3CCC(O)C3)cc2F)cc1F]

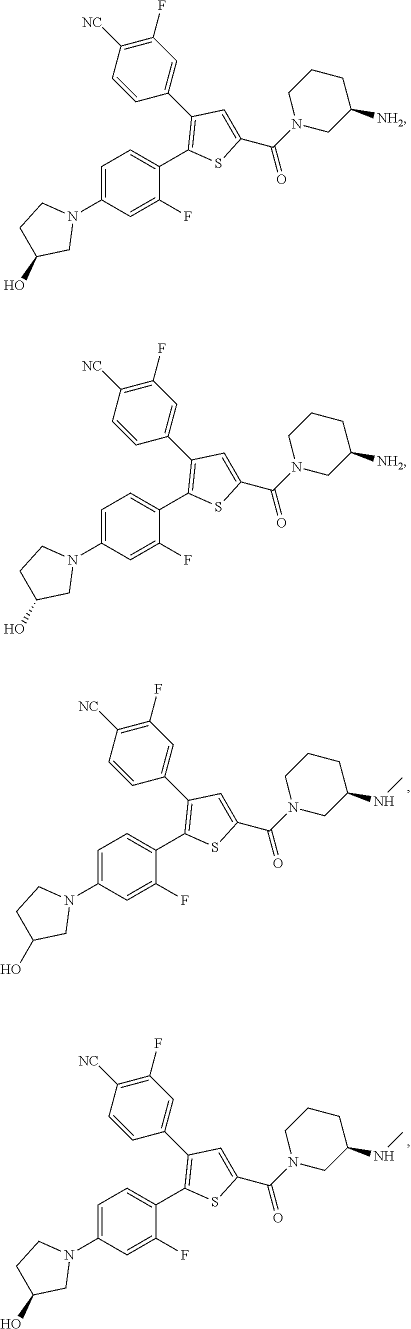 [SMILES: N#Cc1ccc(-c2cc(C(=O)N3CCC[C@@H](N)C3)sc2-c2ccc(N3CC[C@H](O)C3)cc2F)cc1F ; N#Cc1ccc(-c2cc(C(=O)N3CCC[C@@H](N)C3)sc2-c2ccc(N3CC[C@@H](O)C3)cc2F)cc1F ; CN[C@@H]1CCCN(C(=O)c2cc(-c3ccc(C#N)c(F)c3)c(-c3ccc(N4CCC(O)C4)cc3F)s2)C1 ; CN[C@@H]1CCCN(C(=O)c2cc(-c3ccc(C#N)c(F)c3)c(-c3ccc(N4CC[C@H](O)C4)cc3F)s2)C1]

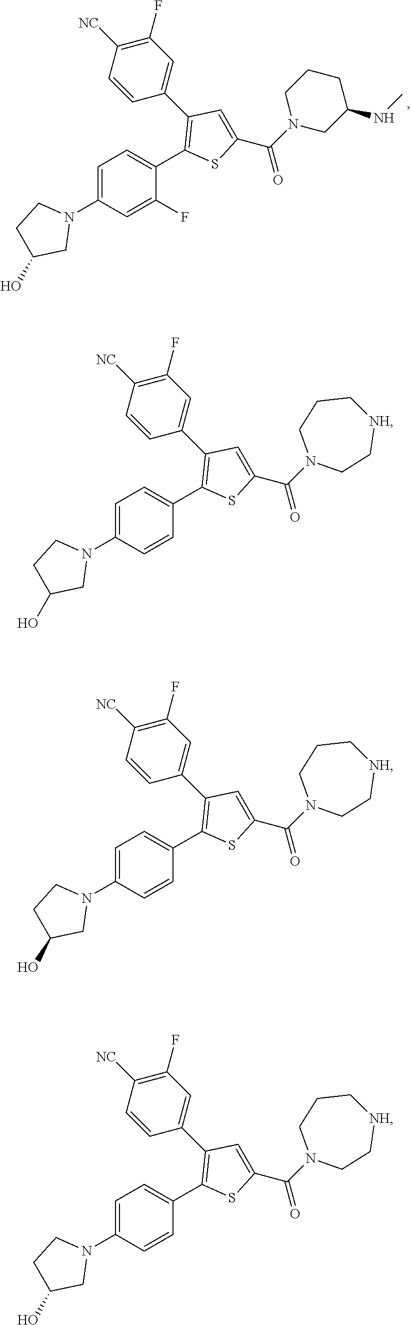 [SMILES: CN[C@@H]1CCCN(C(=O)c2cc(-c3ccc(C#N)c(F)c3)c(-c3ccc(N4CC[C@@H](O)C4)cc3F)s2)C1 ; N#Cc1ccc(-c2cc(C(=O)N3CCCNCC3)sc2-c2ccc(N3CCC(O)C3)cc2)cc1F ; N#Cc1ccc(-c2cc(C(=O)N3CCCNCC3)sc2-c2ccc(N3CC[C@H](O)C3)cc2)cc1F ; N#Cc1ccc(-c2cc(C(=O)N3CCCNCC3)sc2-c2ccc(N3CC[C@@H](O)C3)cc2)cc1F]

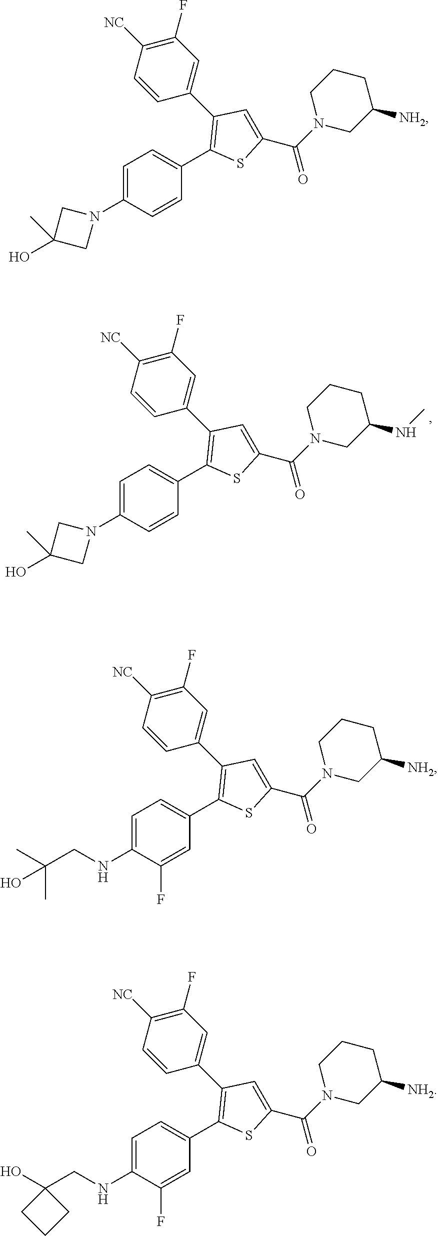

The present disclosure also provides the compound or the pharmaceutically acceptable salt thereof, wherein the salt is hydrochloride.

The present disclosure also provides a use of the compound or the pharmaceutically acceptable salt thereof in the manufacture of a medicament for treating diseases related to LSD1.

### Technical Effect

The compounds of the present disclosure can better inhibit the activity of LSD1; the compounds also have obvious inhibitory activity on the proliferation of NCI-H1417 cells, and have good pharmacokinetic properties (including good oral bioavailability, oral exposure, half-life, and clearance rate, etc.); the compounds do not have significant inhibitory effect on the hERG potassium ion channel, thus ensuring high safety; the compounds of the present disclosure have an excellent tumor inhibitory effect on human small cell lung cancer NCI-H1417 xenograft tumor model.

### Definition and Description

Unless otherwise specified, the following terms and phrases used herein have the following meanings. A specific term or phrase should not be considered indefinite or unclear in the absence of a particular definition, but should be understood in the ordinary sense. When a trade name appears herein, it is intended to refer to its corresponding commodity or active ingredient thereof.

The term “pharmaceutically acceptable” is used herein in terms of those compounds, materials, compositions, and/or dosage forms, which are suitable for use in contact with human and animal tissues within the scope of reliable medical judgment, with no excessive toxicity, irritation, an allergic reaction, or other problems or complications, commensurate with a reasonable benefit/risk ratio.

The term “pharmaceutically acceptable salt” refers to a salt of the compound of the present disclosure that is prepared by reacting the compound having a specific substituent of the present disclosure with a relatively non-toxic acid or base. When the compound of the present disclosure contains a relatively acidic functional group, a base addition salt can be obtained by contacting the compound with a sufficient amount of a base in a pure solution or a suitable inert solvent. The pharmaceutically acceptable base addition salt includes a salt of sodium, potassium, calcium, ammonium, organic amine, magnesium, or similar salts. When the compound of the present disclosure contains a relatively basic functional group, an acid addition salt can be obtained by contacting the compound with a sufficient amount of acid in a pure solution or a suitable inert solvent. Examples of the pharmaceutically acceptable acid addition salt include an inorganic acid salt, wherein the inorganic acid includes, for example, hydrochloric acid, hydrobromic acid, nitric acid, carbonic acid, bicarbonate, phosphoric acid, monohydrogen phosphate, dihydrogen phosphate, sulfuric acid, hydrogen sulfate, hydroiodic acid, phosphorous acid; and an organic acid salt, wherein the organic acid includes, for example, acetic acid, propionic acid, isobutyric acid, maleic acid, malonic acid, benzoic acid, succinic acid, suberic acid, fumaric acid, lactic acid, mandelic acid, phthalic acid, benzenesulfonic acid, p-toluenesulfonic acid, citric acid, tartaric acid, and methanesulfonic acid; and salts of an amino acid (such as arginine), and a salt of an organic acid such as glucuronic acid. Certain specific compounds of the present disclosure contain both basic and acidic functional groups and thus can be converted to any base or acid addition salt.

The pharmaceutically acceptable salt of the present disclosure can be prepared from the parent compound that contains an acidic or basic moiety by conventional chemical methods. Generally, such salt can be prepared by reacting the free acid or base form of the compound with a stoichiometric amount of an appropriate base or acid in water or an organic solvent or a mixture thereof.

The compounds of the present disclosure may exist in specific geometric or stereoisomeric forms. The present disclosure contemplates all such compounds, including cis and trans isomers, (−)- and (+)-enantiomers, (R)- and (S)-enantiomers, diastereoisomers, (D)-isomers, (L)-isomers, racemic, and other mixtures thereof, such as enantiomers or diastereomer enriched mixtures, all of which are within the scope of the present disclosure. Additional asymmetric carbon atoms may be present in substituents such as alkyl. All these isomers and their mixtures are encompassed within the scope of the present disclosure.

Unless otherwise specified, the term “enantiomer” or “optical isomer” refers to stereoisomers that are mirror images of each other.

Unless otherwise specified, the term “cis-trans isomer” or “geometric isomer” is caused by the inability to rotate freely of double bonds or single bonds of ring-forming carbon atoms.

Unless otherwise specified, the term “diastereomer” refers to a stereoisomer in which a molecule has two or more chiral centers and the relationship between the molecules is not mirror images.

Unless otherwise specified, “(+)” refers to dextrorotation, “(−)” refers to levorotation, and “(±)” refers to racemic.

Unless otherwise specified, the absolute configuration of a stereogenic center is represented by a wedged solid bond ( [symbol] ) and a wedged dashed bond ( [symbol] ), and the relative configuration of a stereogenic center is represented by a straight solid bond ( [symbol] ) and a straight dashed bond ( [symbol] ), a wave line ( [symbol] ) is used to represent a wedged solid bond ( [symbol] ) or a wedged dashed bond ( [symbol] ), or the wave line ( [symbol] ) is used to represent a straight solid bond ( [symbol] ) and a straight dashed bond ( [symbol] ).

Unless otherwise specified, when double bond structure, such as carbon-carbon double bond, carbon-nitrogen double bond, and nitrogen-nitrogen double bond, exists in the compound, and each of the atoms on the double bond is connected to two different substituents (including the condition where a double bond contains a nitrogen atom, the lone pair of electrons attached on the nitrogen atom is regarded as a substituent connected), if the atom on the double bond in the compound is connected to its substituent by a wave line ( [symbol] ), this refers [symbol] to the (Z) isomer, (E) isomer, or a mixture of two isomers of the compound. For example, the following formula (A) means that the compound exists as a single isomer of formula (A-1) or formula (A-2) or as a mixture of two isomers of formula (A-1) and formula (A-2); the following formula (B) means that the compound exists in the form of a single isomer of formula (B-1) or formula (B-2) or in the form of a mixture of two isomers of formula (B-1) and formula (B-2). The following formula (C) means that the compound exists as a single isomer of formula (C-1) or formula (C-2) or as a mixture of two isomers of formula (C-1) and formula (C-2).

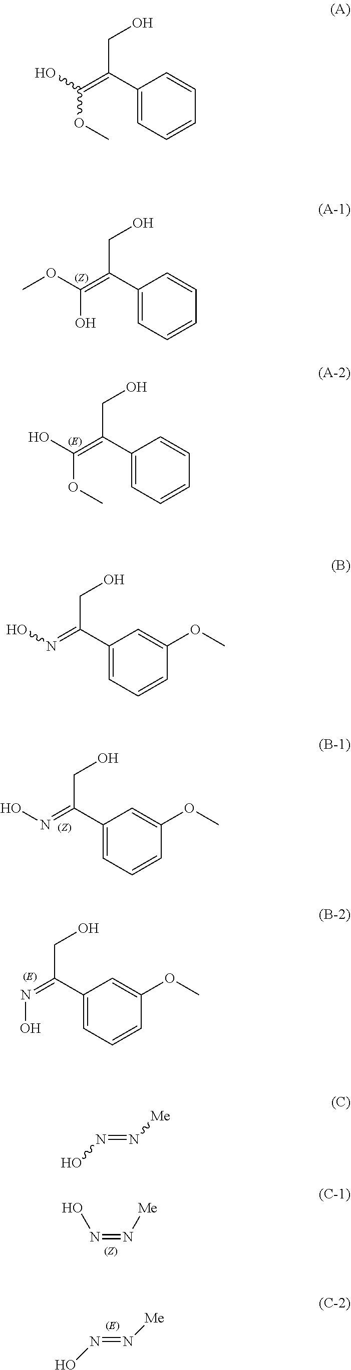

Unless otherwise specified, the term “tautomer” or “tautomeric form” means that at room temperature, the isomers of different functional groups are in dynamic equilibrium and can be transformed into each other quickly. If tautomers possibly exist (such as in solution), the chemical equilibrium of tautomers can be reached. For example, proton tautomer (also called prototropic tautomer) includes interconversion through proton migration, such as keto-enol isomerization and imine-enamine isomerization. Valence tautomer includes some recombination of bonding electrons for mutual transformation. A specific example of keto-enol tautomerization is the tautomerism between two tautomers of pentane-2,4-dione and 4-hydroxypent-3-en-2-one.

Unless otherwise specified, the terms “enriched in one isomer”, “enriched in isomers”, “enriched in one enantiomer”, or “enriched in enantiomers” refer to the content of one of the isomers or enantiomers is less than 100%, and the content of the isomer or enantiomer is greater than or equal to 60%, or greater than or equal to 70%, or greater than or equal to 80%, or greater than or equal to 90%, or greater than or equal to 95%, or greater than or equal to 96%, or greater than or equal to 97%, or greater than or equal to 98%, or greater than or equal to 99%, or greater than or equal to 99.5%, or greater than or equal to 99.6%, or greater than or equal to 99.7%, or greater than or equal to 99.8%, or greater than or equal to 99.9%.

Unless otherwise specified, the term “isomer excess” or “enantiomeric excess” refers to the difference between the relative percentages of two isomers or two enantiomers. For example, if the content of one isomer or enantiomer is 90%, and the content of the other isomer or enantiomer is 10%, the isomer or enantiomer excess (ee value) is 80%.

Optically active (R)- and (S)-isomers and D and L isomers can be prepared using chiral synthesis or chiral reagents or other conventional techniques. If one kind of enantiomer of a certain compound of the present disclosure is to be obtained, the pure desired enantiomer can be obtained by asymmetric synthesis or derivative action of chiral auxiliary followed by separating the resulting diastereomeric mixture and cleaving the auxiliary group. Alternatively, when the molecule contains a basic functional group (such as amino) or an acidic functional group (such as carboxyl), the compound reacts with an appropriate optically active acid or base to form a salt of the diastereomeric isomer which is then subjected to diastereomeric resolution through the conventional method in the art to give the pure enantiomer. In addition, the enantiomer and the diastereoisomer are generally isolated through chromatography which uses a chiral stationary phase and optionally combines with a chemical derivative method (such as carbamate generated from amine).

The compound of the present disclosure may contain an unnatural proportion of atomic isotope at one or more than one atom that constitutes the compound. For example, the compound can be radiolabeled with a radioactive isotope, such as tritium (H), iodine-125 (¹²⁵I), or C-14 (¹⁴C). For another example, deuterated drugs can be formed by replacing hydrogen with deuterium, the bond formed by deuterium and carbon is stronger than that of ordinary hydrogen and carbon, compared with non-deuterated drugs, deuterated drugs have the advantages of reduced toxic and side effects, increased drug stability, enhanced efficacy, extended biological half-life of drugs, etc. All isotopic variations of the compound of the present disclosure, whether radioactive or not, are encompassed within the scope of the present disclosure.

The term “optional” or “optionally” means that the subsequently described event or circumstance may, but does not necessarily, occur, and the description includes instances where the event or circumstance occurs and instances where it does not.

The term “substituted” means one or more than one hydrogen atom on a specific atom is substituted by the substituent, including deuterium and hydrogen variables, as long as the valence of the specific atom is normal and the substituted compound is stable. When the substituent is an oxygen (i.e., ═O), it means two hydrogen atoms are substituted. Positions on an aromatic ring cannot be substituted with a ketone.

The term “optionally substituted” means an atom can be substituted by a substituent or not, unless otherwise specified, the type and number of the substituent may be arbitrary as long as it is chemically achievable.

When any variable (such as R) occurs in the constitution or structure of the compound more than once, the definition of the variable at each occurrence is independent. Thus, for example, if a group is substituted with 0 to 2 R, the group can be optionally substituted with up to two R, wherein the definition of R at each occurrence is independent. Moreover, a combination of the substituent and/or the variant thereof is allowed only when the combination results in a stable compound.

When the number of a linking group is 0, such as —(CRR)₀—, it means that the linking group is a single bond.

When one of the variables is selected from a single bond, it means that the two groups linked by the single bond are connected directly. For example, when L in A-L-Z represents a single bond, the structure of A-L-Z is actually A-Z.

When a substituent is vacant, it means that the substituent is absent, for example, when X is vacant in A-X, the structure of A-X is actually A. When the enumerative substituent does not indicate by which atom it is linked to the group to be substituted, such substituent can be bonded by any atom thereof. For example, when pyridyl acts as a substituent, it can be linked to the group to be substituted by any carbon atom on the pyridine ring.

When the enumerative linking group does not indicate the direction for linking, the direction for linking is arbitrary, for example, the linking group L contained in

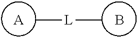

is -M-W—, then -M-W— can link ring A and ring B to form

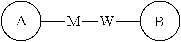

in the direction same as left-to-right reading order, and form

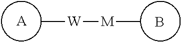

in the direction contrary to left-to-right reading order. A combination of the linking groups, substituents and/or variables thereof is allowed only when such combination can result in a stable compound.

Unless otherwise specified, when a group has one or more linkable sites, any one or more sites of the group can be linked to other groups through chemical bonds. When the linking site of the chemical bond is not positioned, and there is H atom at the linkable site, then the number of H atom at the site will decrease correspondingly with the number of chemical bond(s) linking thereto to meet the corresponding valence. The chemical bond between the site and other groups can be represented by a straight solid bond ( [symbol] ), a straight dashed bond ( [symbol] ) or a wavy line ( [symbol] ). For example, the straight solid bond in —OCH₃ means that it is linked to other groups through the oxygen atom in the group; the straight dashed bond in [symbol] means that it is linked to other groups through the two ends of the nitrogen atom in the group; the wave lines in

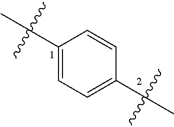 [SMILES: *c1ccc(*)cc1]

means that the phenyl group is linked to other groups through carbon atoms at position 1 and position 2;

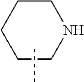

means that it can be linked to other groups through any linkable sites on the piperidinyl by one chemical bond, including at least four types of linkage, including

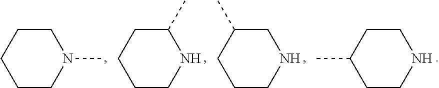

Even though the H atom is drawn on the —N—,

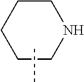

still includes the linkage of

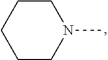

merely when one chemical bond was connected, the H of this site will be reduced by one to the corresponding monovalent piperidinyl.

Unless otherwise specified, the terms “halo” or “halogen” by itself or as a part of another substituent refers to a fluorine, chlorine, bromine, or iodine atom.

Unless otherwise specified, Cₙ₋ₙ₊ₘ or Cₙ-Cₙ₊ₘ includes any specific case of n to n+m carbons, for example, C₁₋₁₂ includes C₁, C₂, C₃, C₄, C₅, C₆, C₇, C₈, C₉, C₁₀, C₁₁, and C₁₂, and any range from n to n+m is also included, for example C₁₋₁₂ includes C₁₋₃, C₁₋₆, C₁₋₉, C₃₋₆, C₃₋₉, C₃₋₁₂, C₆₋₉, C₆₋₁₂, and C₉₋₁₂; similarly, n-membered to n+m-membered means that the number of atoms on the ring is from n to n+m, for example, 3- to 12-membered ring includes 3-membered ring, 4-membered ring, 5-membered ring, 6-membered ring, 7-membered ring, 8-membered ring, 9-membered ring, 10-membered ring, 11-membered ring, and 12-membered ring, and any range from n to n+m is also included, for example, 3- to 12-membered ring includes 3- to 6-membered ring, 3- to 9-membered ring, 5- to 6-membered ring, 5- to 7-membered ring, 6- to 7-membered ring, 6- to 8-membered ring, and 6- to 10-membered ring.

Unless otherwise specified, the term “C₁₋₄ alkyl” refers to a linear or branched saturated hydrocarbon group consisting of 1 to 4 carbon atoms. The C₁₋₄ alkyl includes C₁₋₂, C₁₋₃, C₂₋₃ alkyl, etc.; it can be monovalent (such as methyl), divalent (such as methylene), or multivalent (such as methine). Examples of C₁₋₄ alkyl include, but are not limited to, methyl (Me), ethyl (Et), propyl (including n-propyl and isopropyl), butyl (including n-butyl, isobutyl, s-butyl, and t-butyl), etc.

Unless otherwise specified, the term “C₁₋₃ alkyl” refers to a linear or branched saturated hydrocarbon group consisting of 1 to 3 carbon atoms. The C₁₋₃ alkyl includes C₁₋₂, C₂₋₃ alkyl, etc.; it can be monovalent (such as methyl), divalent (such as methylene), or multivalent (such as methine). Examples of C₁₋₃ alkyl include, but are not limited to, methyl (Me), ethyl (Et), propyl (including n-propyl and isopropyl), etc.

Unless otherwise specified, the term “C₁₋₃ alkoxy” refers to an alkyl group containing 1 to 3 carbon atoms that are connected to the rest of the molecule through an oxygen atom. The C₁₋₃ alkoxy includes C₁₋₂, C₂₋₃, C₃, C₂ alkoxy, etc. Examples of C₁₋₃ alkoxy include, but are not limited to, methoxy, ethoxy, propoxy (including n-propoxy and isopropoxy), etc.

Unless otherwise specified, “C₃₋₆ cycloalkyl” refers to a saturated cyclic hydrocarbon group consisting of 3 to 6 carbon atoms, which is monocyclic system and bicyclic systems, and the C₃₋₆ cycloalkyl includes C₃₋₆, C₄₋₅, C₅₋₆ cycloalkyl, etc.; it can be monovalent, divalent, or multivalent. Examples of C₃₋₆ cycloalkyl include, but are not limited to, cyclopropyl, cyclobutyl, cyclopentyl, cyclohexyl, etc.

Unless otherwise specified, the term “3- to 6-membered heterocycloalkyl” by itself or in combination with other terms refers to a saturated cyclic group consisting of 3 to 6 ring atoms, wherein 1, 2, 3, or 4 ring atoms are heteroatoms independently selected from O, S, and N, and the rest are carbon atoms, wherein nitrogen atoms are optionally quaternized, and nitrogen and sulfur heteroatoms can be optionally oxidized (i.e., NO and S(O)ₚ, p is 1 or 2). It includes monocyclic and bicyclic systems, wherein the bicyclic systems include a spiro ring, a fused ring, and a bridged ring. In addition, concerning the “3- to 6-membered heterocycloalkyl”, a heteroatom may occupy the connection position of the heterocycloalkyl with the rest of the molecule. The 3- to 6-membered heterocycloalkyl includes 4- to 6-membered, 5- to 6-membered, 4-membered, 5-membered, 6-membered heterocycloalkyl, etc. Examples of 3- to 6-membered heterocycloalkyl include, but are not limited to, azetidinyl, oxetanyl, thietanyl, pyrrolidinyl, pyrazolidinyl, imidazolidinyl, tetrahydrothienyl (including tetrahydrothiophen-2-yl, tetrahydrothiophen-3-yl, etc.), tetrahydrofuranyl (including tetrahydrofuran-2-yl, etc.), tetrahydropyranyl, piperidinyl (including 1-piperidinyl, 2-piperidinyl, 3-piperidinyl, etc.), piperazinyl (including 1-piperazinyl, 2-piperazinyl, etc.), morpholinyl (including 3-morpholinyl, 4-morpholinyl, etc.), dioxinyl, dithianyl, isoxazolidinyl, isothiazolidinyl, 1,2-oxazinyl, 1,2-thiazinyl, hexahydropyridazinyl, etc.

The term “leaving group” refers to a functional group or atom which can be replaced by another functional group or atom through a substitution reaction (such as nucleophilic substitution reaction). For example, representative leaving groups include triflate; chlorine, bromine, and iodine; sulfonate group, such as mesylate, tosylate, p-bromobenzenesulfonate, p-toluenesulfonate; acyloxy, such as acetoxy, trifluoroacetoxy.

The term “protecting group” includes, but is not limited to, “amino protecting group”, “hydroxyl protecting group”, or “mercapto protecting group”. The term “amino protecting group” refers to a protecting group suitable for preventing the side reactions occurring at the nitrogen of an amino. Representative amino protecting groups include, but are not limited to: formyl; acyl, such as alkanoyl (e.g., acetyl, trichloroacetyl, or trifluoroacetyl); alkoxycarbonyl, such as tert-butoxycarbonyl (Boc); arylmethoxycarbonyl such as benzyloxycarbonyl (Cbz) and 9-fluorenylmethoxycarbonyl (Fmoc); arylmethyl, such as benzyl (Bn), trityl (Tr), 1,1-bis-(4′-methoxyphenyl)methyl; silyl, such as trimethylsilyl (TMS) and tert-butyldimethylsilyl (TBS). The term “hydroxyl protecting group” refers to a protecting group suitable for blocking the side reaction on hydroxyl. Representative hydroxyl protecting groups include, but are not limited to: alkyl, such as methyl, ethyl, and tert-butyl; acyl, such as alkanoyl (e.g., acetyl); arylmethyl, such as benzyl (Bn), p-methoxybenzyl (PMB), 9-fluorenylmethyl (Fm), and diphenylmethyl (benzhydryl, DPM); silyl, such as trimethylsilyl (TMS) and tert-butyl dimethyl silyl (TBS).

The compounds of the present disclosure can be prepared by a variety of synthetic methods known to those skilled in the art, including the specific embodiments listed below, the embodiments formed by their combination with other chemical synthesis methods, and equivalent alternatives known to those skilled in the art, preferred embodiments include but are not limited to the examples of the present disclosure.

The structure of the compounds of the present disclosure can be confirmed by conventional methods known to those skilled in the art, and if the present disclosure involves an absolute configuration of a compound, then the absolute configuration can be confirmed using conventional techniques in the art. For example, in the case of single crystal X-ray diffraction (SXRD), the absolute configuration can be confirmed by collecting diffraction intensity data from the cultured single crystal using a Bruker D8 venture diffractometer with CuKα radiation as the light source and scanning mode: φ/ω scan, and after collecting the relevant data, the crystal structure can be further analyzed by the direct method (Shelxs97).

The solvents used in the present disclosure are commercially available. The present disclosure adopts the following abbreviations: aq represents water; m-CPBA represents 3-chloroperoxybenzoic acid; eq represents equivalent; Boc represents tert-butoxycarbonyl, which is an amine protecting group.

The compounds of the present disclosure are named according to the conventional naming principles in the art or by ChemDraw® software, and the commercially available compounds use the supplier catalog names.

### DETAILED DESCRIPTION OF THE PREFERRED EMBODIMENT

The present disclosure is described in detail by the examples below, but it does not mean that there are any adverse restrictions on the present disclosure. The present disclosure has been described in detail herein, and specific embodiments thereof have also been disclosed; for those skilled in the art, it is obvious to make various modifications and improvements to the embodiments of the present disclosure without departing from the spirit and scope of the present disclosure.

### Example 1

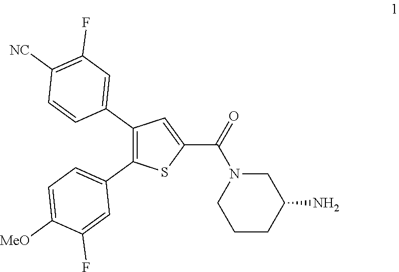 [SMILES: COc1ccc(-c2sc(C(=O)N3CCC[C@@H](N)C3)cc2-c2ccc(C#N)c(F)c2)cc1F]

Synthetic Route:

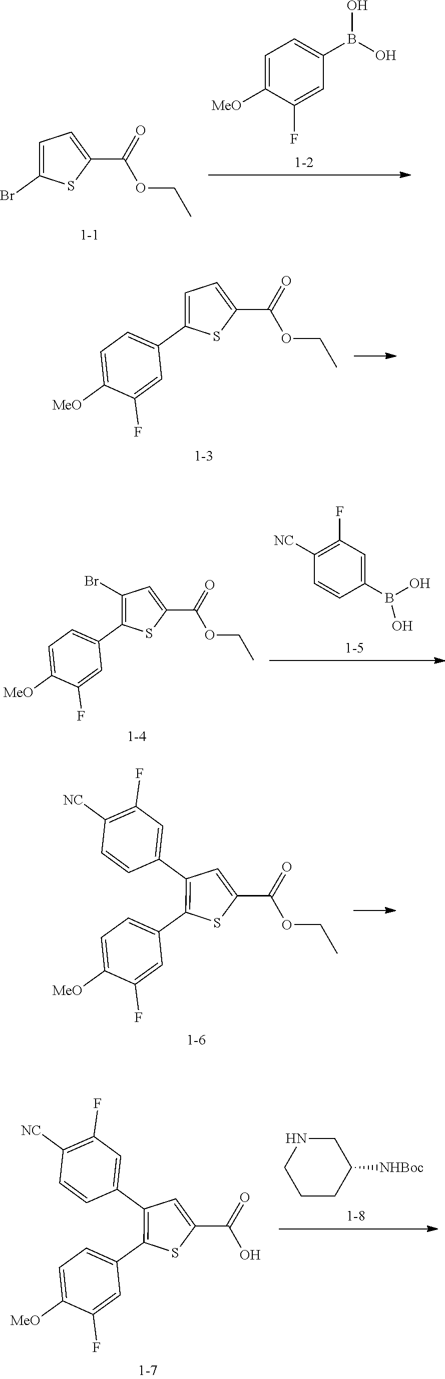

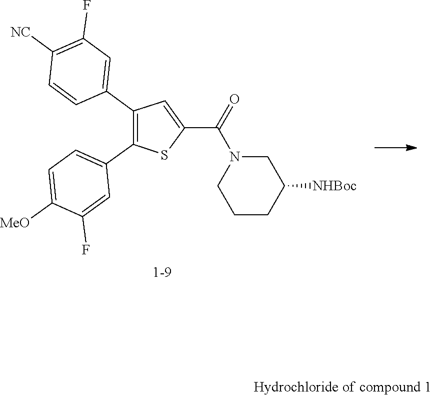 [SMILES: COc1ccc(-c2sc(C(=O)N3CCC[C@@H](NC(=O)OC(C)(C)C)C3)cc2-c2ccc(C#N)c(F)c2)cc1F]

### Step 1

Under nitrogen atmosphere, compound 1-1 (100 mg, 0.425 mmol), compound 1-2 (108 mg, 0.638 mmol), and sodium carbonate (90.2 mg, 0.851 mmol) were dissolved in dioxane (3.00 mL) and water (3.00 mL), and the reaction mixture was added with tetrakis(triphenylphosphine)palladium (49.2 mg, 42.5 μmol), stirred, and reacted for 14 hours at 80° C. The reaction mixture was concentrated under reduced pressure, and the residue was purified by silica gel column chromatography (10/1, petroleum ether/ethyl acetate, R_{f}=0.35) to obtain compound 1-3. ¹H NMR (400 MHz, CDCl₃) δ=7.74 (d, J=4.0 Hz, 1H), 7.42-7.37 (m, 1H), 7.37-7.33 (m, 1H), 7.18 (d, J=4.0 Hz, 1H), 7.03-6.96 (m, 1H), 4.37 (q, J=7.2 Hz, 2H), 3.94 (s, 3H), 1.40 (t, J=7.2 Hz, 3H). MS-ESI calculated for [M+H]⁺ 281, found 281.

### Step 2

Compound 1-3 (580 mg, 2.07 mmol) was dissolved in N,N-dimethylformamide (5.00 mL). N-Bromosuccinimide (737 mg, 4.14 mmol) was added thereto, and the reaction mixture was stirred and reacted for 14 hours at 25° C. After the reaction mixture was concentrated, the resulting crude product was purified by silica gel column chromatography (5/1, petroleum ether/ethyl acetate, R_{f}=0.7) to obtain compound 1-4. ¹H NMR (400 MHz, CDCl₃) δ=7.71 (s, 1H), 7.48-7.45 (m, 1H), 7.43-7.36 (m, 1H), 7.04 (t, J=8.6 Hz, 1H), 4.38 (q, J=7.2 Hz, 2H), 3.96 (s, 3H), 1.39 (t, J=7.2 Hz, 3H). MS-ESI calculated for [M+H]⁺ 359, found 359.

### Step 3

Under nitrogen atmosphere, compound 1-4 (710 mg, 1.98 mmol), compound 1-5 (391 mg, 2.37 mmol), and sodium carbonate (419 mg, 3.95 mmol) were dissolved in ethanol (4.00 mL) and water (1.00 mL), and the reaction mixture was added with bis(di-tert-butyl(4-dimethylaminophenyl)phosphine)dichloropalladium(II) (70.0 mg, 98.8 μmol), stirred, and reacted for 12 hours at 80° C. After the reaction mixture was concentrated, the resulting crude product was purified by silica gel column chromatography (10/1, petroleum ether/ethyl acetate, R_{f}=0.6) to obtain compound 1-6. ¹H NMR (400 MHz, CDCl₃) δ=7.80 (s, 1H), 7.58-7.55 (m, 1H), 7.19-7.10 (m, 2H), 7.04-6.98 (m, 2H), 6.96-6.91 (m, 1H), 4.40 (q, J=7.2 Hz, 2H), 3.93 (s, 3H), 1.41 (t, J=7.2 Hz, 3H). MS-ESI calculated for [M+H]⁺ 400, found 400.

### Step 4

Compound 1-6 (350 mg, 876 μmol) was dissolved in tetrahydrofuran (3.00 mL) and water (3.00 mL), and lithium hydroxide monohydrate (110 mg, 2.63 mmol) was added thereto, and the reaction mixture was stirred and reacted for 14 hours at 20° C. After the reaction mixture was concentrated, the residue was added with water (10 mL). The mixture was acidified to pH=6 to 7 with 1 M dilute hydrochloric acid, and extracted with ethyl acetate (5 mL×3). The organic phases were combined, dried over anhydrous sodium sulfate, filtered, and concentrated to obtain crude compound 1-7, which was used directly in the next reaction step. ¹H NMR (400 MHz, DMSO-d₆) δ=7.90 (s, 1H), 7.89-7.85 (m, 1H), 7.50-7.52 (m, 1H), 7.26-7.16 (m, 3H), 7.10-7.08 (m, 1H), 3.86 (s, 3H). MS-ESI calculated for [M−H]⁺ 370, found 370.

### Step 5

Compound 1-7 (200 mg, 539 μmol), compound 1-8 (108 mg, 539 μmol), and 2-(7-azabenzotriazol-1-yl)-N,N,N′,N′-tetramethyluronium hexafluorophosphate (307 mg, 808 μmol) were dissolved in N,N-dimethylformamide (3.00 mL), and N,N-diisopropylethylamine (209 mg, 1.62 mmol) was added thereto, and the reaction mixture was stirred and reacted for 12 hours at 20° C. To the reaction mixture were added water (20 mL) and ethyl acetate (5 mL). The organic phase was separated, and the aqueous phase was extracted with ethyl acetate (5 mL×3). The organic phases were combined, dried over anhydrous sodium sulfate, filtered, and concentrated, and the resulting crude product was purified by column chromatography (20/1, dichloromethane/methanol, R_{f}=0.5) to obtain compound 1-9. ¹H NMR (400 MHz, CDCl₃) δ=7.72 (s, 1H), 7.58-7.41 (m, 2H), 7.24 (d, J=8.0 Hz, 1H), 7.08-6.98 (m, 2H), 6.96-6.89 (m, 1H), 4.61-4.60 (m, 2H), 4.40-4.36 (m, 1H), 3.93 (s, 3H), 3.74-3.71 (m, 1H), 3.11-2.89 (m, 2H), 2.13-2.04 (m, 1H), 1.93-1.82 (m, 1H), 1.76-1.61 (m, 1H), 1.47 (s, 9H). MS-ESI calculated for [M+H]⁺ 554, found 554.

### Step 6

Compound 1-9 (150 mg, 271 μmol) was dissolved in ethyl acetate (1.00 mL), and hydrochloride ethyl acetate solution (4.00 mL, 4M) was added thereto, and the reaction mixture was stirred and reacted for 10 hours at 20° C. After the reaction mixture was concentrated, the residue was purified by preparative high performance liquid chromatography (3_Phenomenex Luna C18 75*30 mm*3 μm; mobile phase A: hydrochloric acid aqueous solution with a volume fraction of 0.05%; mobile phase B: acetonitrile; B (acetonitrile) %: 25% to 45%, 6.5 minutes) to obtain the hydrochloride of compound 1. ¹H NMR (400 MHz, DMSO-d₆) δ=8.15 (s, 2H), 7.89 (t, J=7.2 Hz, 1H), 7.70-7.54 (m, 2H), 7.30-7.15 (m, 3H), 7.09-7.07 (m, 1H), 4.43-4.25 (m, 1H), 4.16-3.96 (m, 1H), 3.86 (s, 3H), 3.31-3.20 (m, 3H), 2.13-1.98 (m, 1H), 1.90-1.74 (m, 1H), 1.72-1.47 (m, 2H). MS-ESI calculated for [M+H]⁺ 454, found 454.

### Example 2

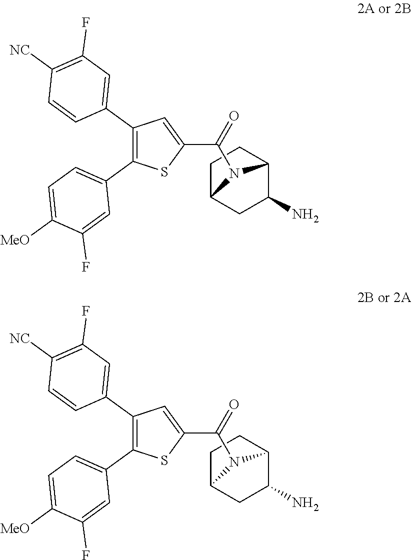

Synthetic Route:

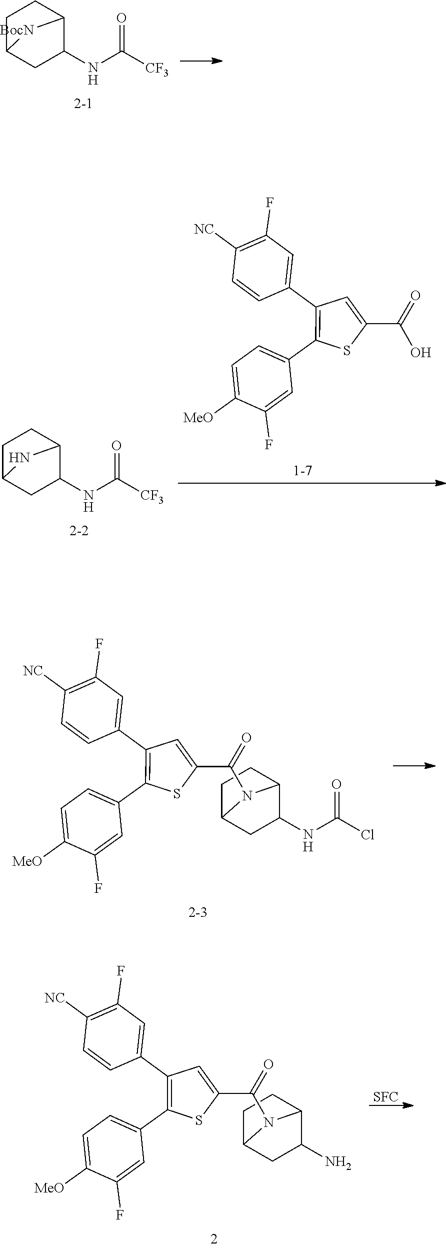 [SMILES: CC(C)(C)OC(=O)N1C2CCC1C(NC(=O)C(F)(F)F)C2 ; O=C(NC1CC2CCC1N2)C(F)(F)F ; COc1ccc(-c2sc(C(=O)O)cc2-c2ccc(C#N)c(F)c2)cc1F ; COc1ccc(-c2sc(C(=O)N3C4CCC3C(NC(=O)Cl)C4)cc2-c2ccc(C#N)c(F)c2)cc1F ; COc1ccc(-c2sc(C(=O)N3C4CCC3C(N)C4)cc2-c2ccc(C#N)c(F)c2)cc1F ; "SFC"]

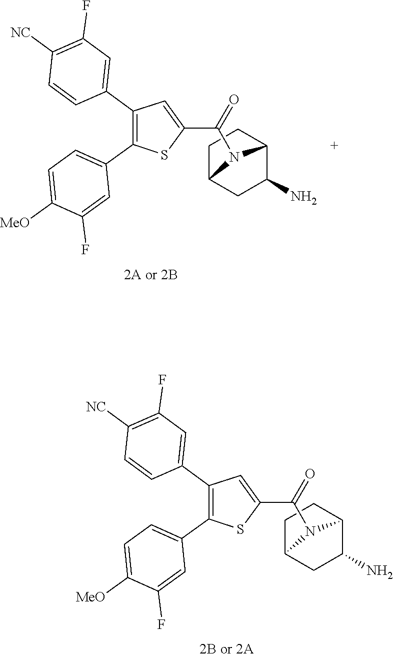 [SMILES: COc1ccc(-c2sc(C(=O)N3[C@H]4CC[C@@H]3[C@@H](N)C4)cc2-c2ccc(C#N)c(F)c2)cc1F ; COc1ccc(-c2sc(C(=O)N3[C@@H]4CC[C@H]3[C@H](N)C4)cc2-c2ccc(C#N)c(F)c2)cc1F]

### Step 1

Compound 2-1 (150 mg, 487 μmol) was dissolved in ethyl acetate (1 mL), and hydrochloride ethyl acetate solution (5.00 mL, 4M) was added thereto, and the reaction mixture was stirred and reacted for 14 hours at 20° C. After the reaction mixture was concentrated, the hydrochloride of compound 2-2 was obtained, and the crude product was used directly in the next reaction step without purification. ¹H NMR (400 MHz, DMSO-d₆) δ=9.83 (d, J=6.0 Hz, 1H), 9.18 (s, 1H), 4.43-4.28 (m, 1H), 4.20 (t, J=4.4 Hz, 1H), 4.13 (t, J=4.4 Hz, 1H), 2.29-2.16 (m, 1H), 1.88-1.79 (m, 2H), 1.78-1.65 (m, 3H). MS-ESI calculated for [M+H]⁺ 209, found 209.

### Step 2

The hydrochloride of compound 2-2 (116 mg, 474 μmol), 2-(7-azabenzotriazol-1-yl)-N,N,N′,N′-tetramethyluronium hexafluorophosphate (270 mg, 711 μmol), and N,N-diisopropylethylamine (184 mg, 1.42 mmol) were dissolved in N,N-dimethylformamide (3.00 mL), and compound 1-7 (176 mg, 474 μmol) was added thereto, and the reaction mixture was stirred and reacted for 12 hours at 20° C. To the reaction mixture were added water (20 mL) and ethyl acetate (5 mL). The organic phase was separated, and the aqueous phase was extracted with ethyl acetate (5 mL×3). The organic phases were combined, dried over anhydrous sodium sulfate, filtered, and concentrated, and the resulting crude product was purified by column chromatography (20/1, dichloromethane/methanol, R_{f}=0.65) to obtain compound 2-3. ¹H NMR (400 MHz, CDCl₃) δ=7.57 (dd, J=6.8, 7.6 Hz, 1H), 7.53 (s, 1H), 7.19-7.10 (m, 2H), 7.05-7.00 (m, 1H), 7.00-6.98 (m, 1H), 6.97-6.90 (m, 1H), 6.61 (d, J=4.8 Hz, 1H), 5.00 (t, J=4.8 Hz, 1H), 4.76 (t, J=4.8 Hz, 1H), 4.44-4.30 (m, 1H), 3.93 (s, 3H), 2.65-2.50 (m, 1H), 2.05-1.97 (m, 1H), 1.97-1.79 (m, 2H), 1.67-1.59 (m, 2H). MS-ESI calculated for [M+H]⁺ 562, found 562.

### Step 3

Compound 2-3 (70.0 mg, 125 μmol) was dissolved in tetrahydrofuran (1.00 mL) and water (0.500 mL), then sodium hydroxide (15.0 mg, 374 μmol) was added thereto, and the reaction mixture was stirred and reacted for 14 hours at 50° C. To the reaction mixture were added water (5 mL) and ethyl acetate (2 mL). The organic phase was separated, and the aqueous phase was extracted with ethyl acetate (2 mL×3). The organic phases were combined, dried over anhydrous sodium sulfate, filtered, and concentrated, and the residue was purified by preparative high performance liquid chromatography (3_Phenomenex Luna C18 75*30 mm*3 μm; mobile phase A: hydrochloric acid aqueous solution with a volume fraction of 0.05%; mobile phase B: acetonitrile; B %: 25% to 45%, 6.5 minutes) to obtain the hydrochloride of compound 2. ¹H NMR (400 MHz, DMSO-d₆) δ=8.52 (s, 2H), 7.88 (t, J=7.6 Hz, 1H), 7.84 (s, 1H), 7.58 (d, J=10.4 Hz, 1H), 7.27-7.15 (m, 3H), 7.10-7.08 (m, 1H), 4.79-4.57 (m, 2H), 3.86 (s, 3H), 3.68-3.66 (m, 1H), 2.37-2.20 (m, 1H), 2.04-1.91 (m, 1H), 1.88-1.67 (m, 3H), 1.46 (dd, J=4.0, 12.8 Hz, 1H). MS-ESI calculated for [M+H]⁺ 466, found 466.

### Step 4

Compound 2 was purified by SFC (DAICEL CHIRALPAK IC 250 mm*30 mm, 10 μm; mobile phase A: ammonia aqueous solution with a volume fraction of 0.1%; mobile phase B: methanol; B (methanol) %: 45% to 45%, 20 minutes) to obtain compounds 2A and 2B.

Compound 2A (retention time: 7.579 minutes). Compound 2A ¹H NMR (400 MHz, CD₃OD) δ=7.68 (t, J=7.6 Hz, 1H), 7.63 (s, 1H), 7.33-7.23 (m, 2H), 7.12-7.02 (m, 3H), 4.64-4.56 (m, 2H), 3.89 (s, 3H), 3.51-3.48 (m, 1H), 2.35-2.21 (m, 1H), 2.19-2.16 (m, 1H), 1.90-1.68 (m, 3H), 1.12-1.08 (m, 1H). MS-ESI calculated for [M+H]⁺ 466, found 466.

Compound 2B (retention time: 10.758 minutes). Compound 2B ¹H NMR (400 MHz, CD₃OD) δ=7.73 (s, 1H), 7.69 (t, J=7.4 Hz, 1H), 7.33 (d, J=10 Hz, 1H), 7.26 (d, J=8.0 Hz, 1H), 7.15-7.00 (m, 3H), 4.83-4.75 (m, 2H), 3.89 (s, 3H), 3.86-3.78 (m, 1H), 2.57-2.42 (m, 1H), 2.09-1.91 (m, 3H), 1.85-1.74 (m, 1H), 1.54-1.46 (m, 1H). MS-ESI calculated for [M+H]⁺ 466, found 466.

### Example 3

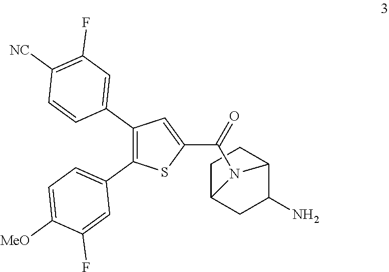 [SMILES: COc1ccc(-c2sc(C(=O)N3C4CCC3C(N)C4)cc2-c2ccc(C#N)c(F)c2)cc1F]

Synthetic Route:

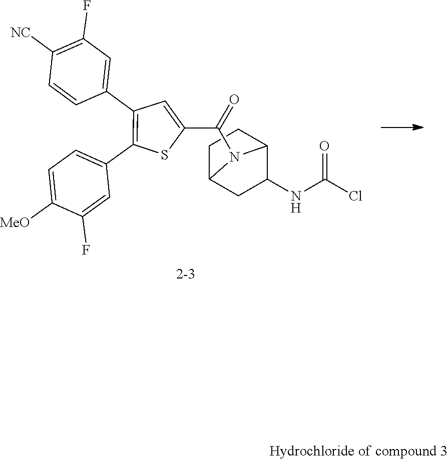 [SMILES: COc1ccc(-c2sc(C(=O)N3C4CCC3C(NC(=O)Cl)C4)cc2-c2ccc(C#N)c(F)c2)cc1F]

### Step 1

Compound 2-3 (130 mg, 232 μmol) was dissolved in methanol (2.50 mL) and water (1.00 mL), then potassium carbonate (166 mg, 1.20 mmol) was added thereto, and the reaction mixture was stirred and reacted for 10 hours at 20° C. After the reaction mixture was concentrated, the residue was purified by preparative high performance liquid chromatography (3_Phenomenex Luna C18 75*30 mm*3 μm; mobile phase A: hydrochloric acid aqueous solution with a volume fraction of 0.05%; mobile phase B: acetonitrile; B %: 25% to 45%, 6.5 minutes) to obtain the hydrochloride of compound 3. ¹H NMR (400 MHz, DMSO-d₆) δ=8.37 (s, 2H), 7.83 (s, 1H), 7.69 (d, J=8.0 Hz, 1H), 7.23-7.14 (m, 3H), 7.08 (dd, J=1.2, 8.0 Hz, 1H), 6.94 (dd, J=1.2, 8.0 Hz, 1H), 4.73-4.66 (m, 2H), 3.85 (s, 3H), 3.83 (s, 3H), 3.67-3.66 (m, 1H), 2.31-2.21 (m, 1H), 1.99-1.90 (m, 1H), 1.87-1.77 (m, 2H), 1.74-1.66 (m, 1H), 1.42 (dd, J=4.4, 12.4 Hz, 1H). MS-ESI calculated for [M+H]⁺ 478, found 478.

### Example 4

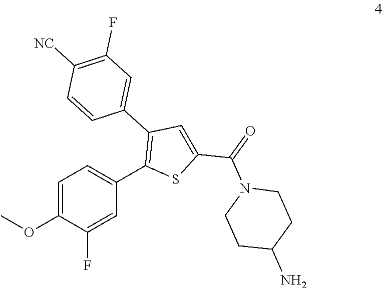 [SMILES: COc1ccc(-c2sc(C(=O)N3CCC(N)CC3)cc2-c2ccc(C#N)c(F)c2)cc1F]

Synthetic Route:

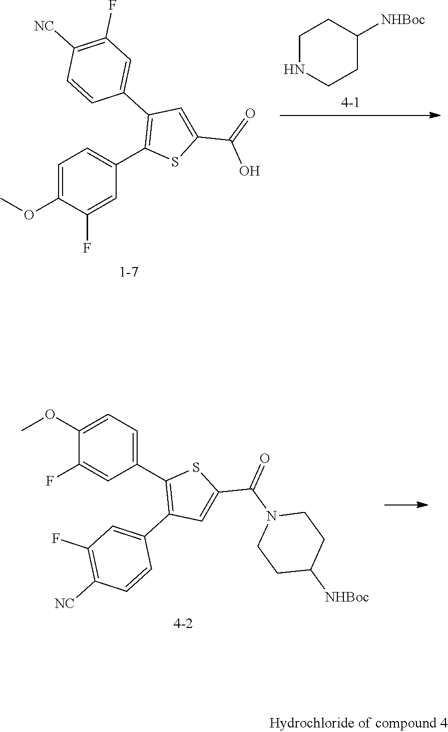 [SMILES: COc1ccc(-c2sc(C(=O)O)cc2-c2ccc(C#N)c(F)c2)cc1F ; CC(C)(C)OC(=O)NC1CCNCC1 ; COc1ccc(-c2sc(C(=O)N3CCC(NC(=O)OC(C)(C)C)CC3)cc2-c2ccc(C#N)c(F)c2)cc1F]

### Step 1

Compound 1-7 (200 mg, 539 μmol), 2-(7-azabenzotriazol-1-yl)-N,N,N′,N′-tetramethyluronium hexafluorophosphate (307 mg, 808 μmol), and N,N-diisopropylethylamine (209 mg, 1.62 mmol) were dissolved in N,N-dimethylformamide (3.00 mL), and then compound 4-1 (108 mg, 539 μmol) was added thereto, and the reaction mixture was stirred and reacted for 12 hours at 20° C. The reaction mixture was added with water (50 mL), and extracted with ethyl acetate (50 mL×2). The organic phase was washed with saturated brine (10 mL), dried over anhydrous sodium sulfate, filtered, and concentrated under reduced pressure. The residue was purified by silica gel column chromatography (1/1, petroleum ether/ethyl acetate, R_{f}=0.53) to obtain compound 4-2. MS-ESI calculated for [M−56+H]⁺ 498, found 498.

### Step 2

Compound 4-2 (310 mg, 559.95 μmol) was dissolved in ethyl acetate (3.00 mL), and hydrochloride ethyl acetate solution (4 mol/L, 2.8 mL) was added thereto, and the reaction mixture was stirred and reacted for 12 hours at 20° C. After the reaction mixture was concentrated, the residue was separated by preparative high performance liquid chromatography (chromatographic column: Venusil ASB Phenyl 150*30 mm*5 μm; mobile phase A: hydrochloric acid aqueous solution with a volume fraction of 0.05%, mobile phase B: acetonitrile B %: 30% to 60%, 9 minutes) to obtain the hydrochloride of compound 4. ¹H NMR (400 MHz, CD₃OD) δ=7.69 (t, J=7.4 Hz, 1H), 7.53 (s, 1H), 7.33 (d, J=10.4 Hz, 1H), 7.25 (d, J=8.0 Hz, 1H), 7.16-6.99 (m, 2H), 4.58 (d, J=12.4 Hz, 2H), 3.90 (s, 3H), 3.50-3.45 (m, 1H), 3.30-3.13 (m, 2H), 2.16-2.13 (m, 2H), 1.73-1.55 (m, 2H). MS-ESI calculated for [M+H]⁺ 454, found 454.

### Example 5

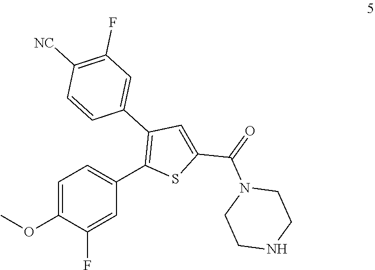 [SMILES: COc1ccc(-c2sc(C(=O)N3CCNCC3)cc2-c2ccc(C#N)c(F)c2)cc1F]

Synthetic Route:

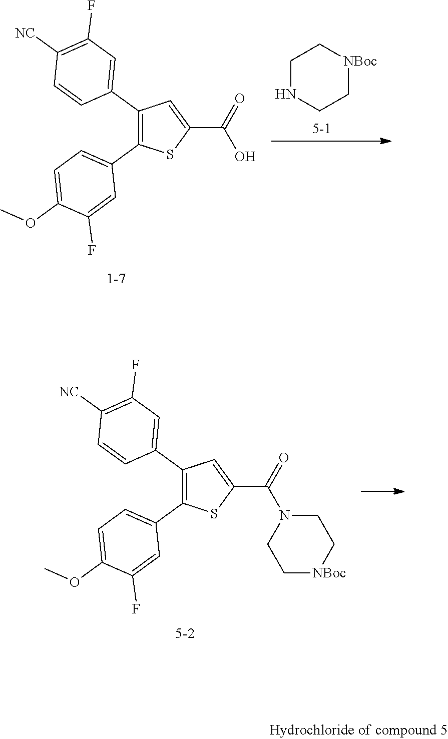 [SMILES: COc1ccc(-c2sc(C(=O)O)cc2-c2ccc(C#N)c(F)c2)cc1F ; CC(C)(C)OC(=O)N1CCNCC1 ; COc1ccc(-c2sc(C(=O)N3CCN(C(=O)OC(C)(C)C)CC3)cc2-c2ccc(C#N)c(F)c2)cc1F]

### Step 1

Compound 1-7 (200 mg, 539 μmol), 2-(7-azabenzotriazol-1-yl)-N,N,N′,N′-tetramethyluronium hexafluorophosphate (307 mg, 808 μmol), and N,N-diisopropylethylamine (209 mg, 1.62 mmol) were dissolved in N,N-dimethylformamide (3.00 mL), and then compound 5-1 (100 mg, 539 μmol) was added thereto, and the reaction mixture was stirred and reacted for 12 hours at 20° C. The reaction mixture was added with water (50 mL), and extracted with ethyl acetate (50 mL×2). The organic phase was washed with saturated brine (10 mL), dried over anhydrous sodium sulfate, filtered, and concentrated under reduced pressure. The residue was purified by silica gel column chromatography (1/1, petroleum ether/ethyl acetate, R_{f}=0.50) to obtain compound 5-2. MS-ESI calculated for [M−56+H]⁺ 484, found 484.

### Step 2

Compound 5-2 (341 mg, 632 μmol) was dissolved in ethyl acetate (3.00 mL), and hydrochloride ethyl acetate solution (4 mol/L, 3.16 mL) was added thereto, and the reaction mixture was stirred and reacted for 12 hours at 20° C. After the reaction mixture was concentrated, the residue was separated by preparative high performance liquid chromatography (chromatographic column: Venusil ASB Phenyl 150*30 mm*5 μm; mobile phase A: hydrochloric acid aqueous solution with a volume fraction of 0.05%; mobile phase B: acetonitrile B %: 30% to 60%, 9 minutes) to obtain the hydrochloride of compound 5. ¹H NMR (400 MHz, CD₃OD) δ=7.70 (t, J=7.2 Hz, 1H), 7.61 (s, 1H), 7.35 (d, J=10.8 Hz, 1H), 7.26 (d, J=8.4 Hz, 1H), 7.16-7.01 (m, 3H), 4.08-4.06 (m, 4H), 3.90 (s, 3H), 3.39-3.33 (m, 4H). MS-ESI calculated for [M+H]⁺ 440, found 440.

### Example 6

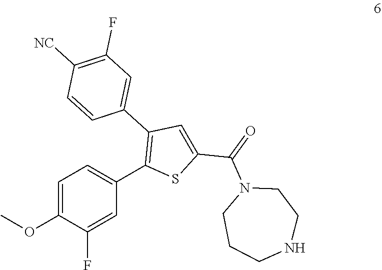 [SMILES: COc1ccc(-c2sc(C(=O)N3CCCNCC3)cc2-c2ccc(C#N)c(F)c2)cc1F]

Synthetic Route:

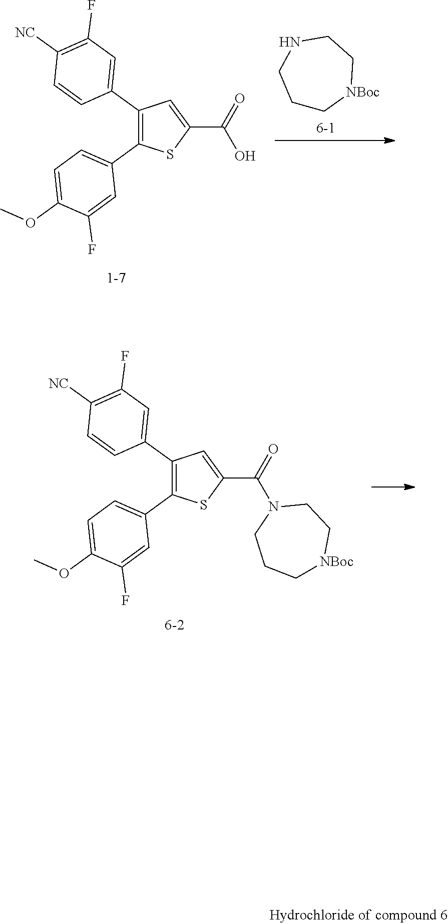 [SMILES: COc1ccc(-c2sc(C(=O)O)cc2-c2ccc(C#N)c(F)c2)cc1F ; CC(C)(C)OC(=O)N1CCCNCC1 ; COc1ccc(-c2sc(C(=O)N3CCCN(C(=O)OC(C)(C)C)CC3)cc2-c2ccc(C#N)c(F)c2)cc1F]

### Step 1

Compound 1-7 (200 mg, 539 μmol), 2-(7-azabenzotriazol-1-yl)-N,N,N′,N′-tetramethyluronium hexafluorophosphate (307 mg, 808 μmol), and N,N-diisopropylethylamine (209 mg, 1.62 mmol) were dissolved in N,N-dimethylformamide (5.00 mL), and then compound 6-1 (108 mg, 539 μmol) was added thereto, and the reaction mixture was stirred and reacted for 12 hours at 20° C. The reaction mixture was added with water (50 mL), and extracted with ethyl acetate (50 mL×2). The organic phase was washed with saturated brine (10 mL), dried over anhydrous sodium sulfate, filtered, and concentrated under reduced pressure. The residue was purified by silica gel column chromatography (1/1, petroleum ether/ethyl acetate, R_{f}=0.49) to obtain compound 6-2. MS-ESI calculated for [M−56+H]⁺ 498, found 498.

### Step 2

Compound 6-2 (404 mg, 729.74 μmol) was dissolved in ethyl acetate (3.00 mL), and hydrochloride ethyl acetate solution (4 mol/L, 3.65 mL) was added thereto, and the reaction mixture was stirred and reacted for 12 hours at 20° C. After the reaction mixture was concentrated, the residue was separated by preparative high performance liquid chromatography (chromatographic column: Venusil ASB Phenyl 150*30 mm*5 μm; mobile phase A: hydrochloric acid aqueous solution with a volume fraction of 0.05%; mobile phase B: acetonitrile B %: 30% to 60%, 9 minutes) to obtain the hydrochloride of compound 6. ¹H NMR (400 MHz, CD₃OD) δ=7.70 (t, J=7.4 Hz, 1H), 7.60 (s, 1H), 7.33 (d, J=9.6 Hz, 1H), 7.26 (d, J=8.0 Hz, 1H), 7.12-7.02 (m, 3H), 3.10-3.90 (m, 4H), 3.90 (s, 3H), 3.50-3.43 (m, 2H), 3.40-3.36 (m, 2H), 2.28-2.20 (m, 2H). MS-ESI calculated for [M+H]⁺ 454, found 454.

### Example 7

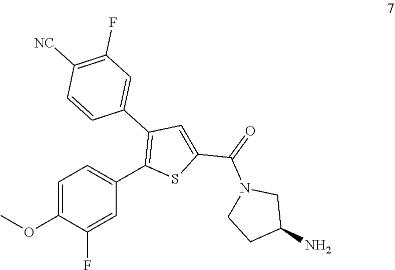 [SMILES: COc1ccc(-c2sc(C(=O)N3CC[C@H](N)C3)cc2-c2ccc(C#N)c(F)c2)cc1F]

Synthetic Route:

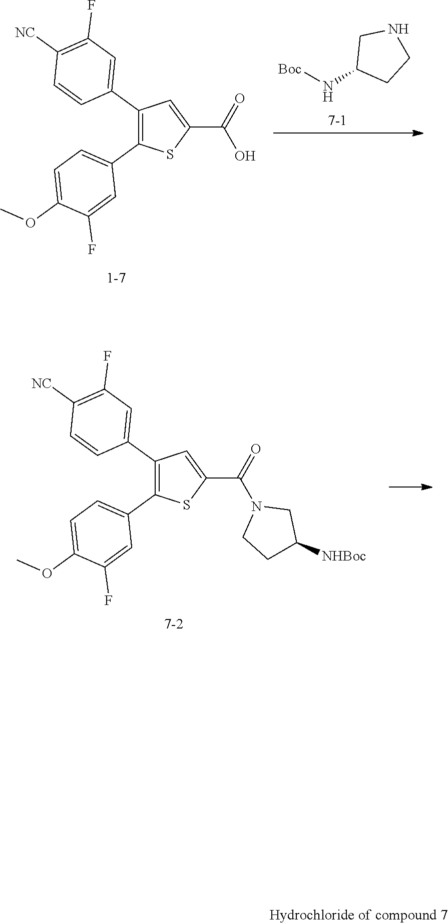 [SMILES: COc1ccc(-c2sc(C(=O)O)cc2-c2ccc(C#N)c(F)c2)cc1F ; CC(C)(C)OC(=O)N[C@H]1CCNC1 ; COc1ccc(-c2sc(C(=O)N3CC[C@H](NC(=O)OC(C)(C)C)C3)cc2-c2ccc(C#N)c(F)c2)cc1F]

### Step 1

Compound 1-7 (200 mg, 539 μmol), 2-(7-azabenzotriazol-1-yl)-N,N,N′,N′-tetramethyluronium hexafluorophosphate (307 mg, 808 μmol), and N,N-diisopropylethylamine (209 mg, 1.62 mmol) were dissolved in N,N-dimethylformamide (5.00 mL), and then compound 7-1 (100 mg, 539 μmol) was added thereto, and the reaction mixture was stirred and reacted for 12 hours at 20° C. The reaction mixture was added with water (50 mL), and extracted with ethyl acetate (50 mL×2). The organic phase was washed with saturated brine (10 mL), dried over anhydrous sodium sulfate, filtered, and concentrated under reduced pressure. The residue was purified by silica gel column chromatography (1/1, petroleum ether/ethyl acetate, R_{f}=0.57) to obtain compound 7-2. MS-ESI calculated for [M−56+H]⁺ 484, found 484.

### Step 2

Compound 7-2 (404 mg, 730 μmol) was dissolved in ethyl acetate (3.00 mL), and hydrochloride ethyl acetate solution (4 mol/L, 3.65 mL) was added thereto, and the reaction mixture was stirred and reacted for 12 hours at 20° C. After the reaction mixture was concentrated, the residue was separated by preparative high performance liquid chromatography (chromatographic column: Venusil ASB Phenyl 150*30 mm*5 μm; mobile phase A: hydrochloric acid aqueous solution with a volume fraction of 0.05%; mobile phase B: acetonitrile; B (acetonitrile) %: 30% to 60%, 9 minutes) to obtain the hydrochloride of compound 7. ¹H NMR (400 MHz, CD₃OD) δ=7.79-7.62 (m, 2H), 7.32 (d, J=10.4 Hz, 1H), 7.26 (dd, J=1.0, 8.2 Hz, 1H), 7.15-7.00 (m, 3H), 4.15-3.98 (m, 2H), 3.90 (s, 3H), 3.85-3.75 (m, 1H), 2.60-2.40 (m, 1H), 2.35-2.10 (m, 1H). MS-ESI calculated for [M+H]⁺ 440, found 440.

### Example 8

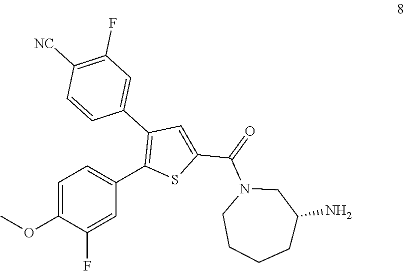 [SMILES: COc1ccc(-c2sc(C(=O)N3CCCC[C@@H](N)C3)cc2-c2ccc(C#N)c(F)c2)cc1F]

Synthetic Route:

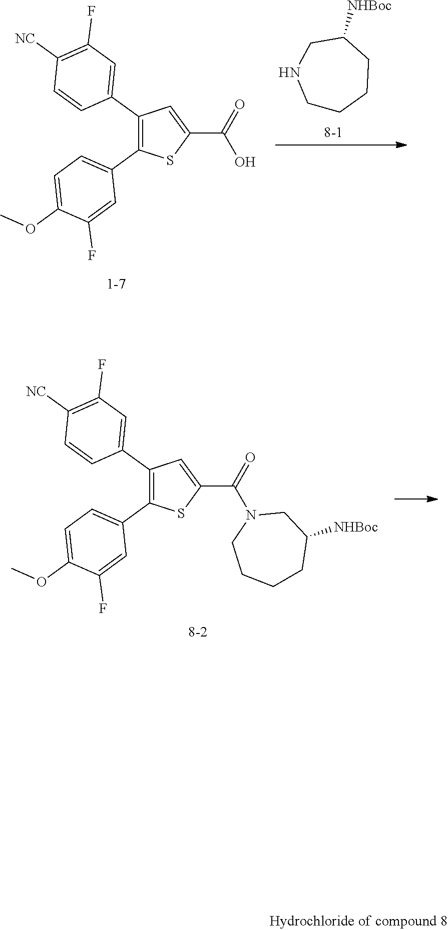 [SMILES: COc1ccc(-c2sc(C(=O)O)cc2-c2ccc(C#N)c(F)c2)cc1F ; CC(C)(C)OC(=O)N[C@@H]1CCCCNC1 ; COc1ccc(-c2sc(C(=O)N3CCCC[C@@H](NC(=O)OC(C)(C)C)C3)cc2-c2ccc(C#N)c(F)c2)cc1F]

### Step 1

Compound 1-7 (150 mg, 404 μmol), 2-(7-azabenzotriazol-1-yl)-N,N,N′,N′-tetramethyluronium hexafluorophosphate (230 mg, 606 μmol), and N,N-diisopropylethylamine (157 mg, 1.21 mmol) were dissolved in N,N-dimethylformamide (5.00 mL), and then compound 8-1 (86.6 mg, 404 μmol) was added thereto, and the reaction mixture was stirred and reacted for 12 hours at 20° C. The reaction mixture was added with water (50 mL), and extracted with ethyl acetate (50 mL×2). The organic phase was washed with saturated brine (10 mL), dried over anhydrous sodium sulfate, filtered, and concentrated under reduced pressure. The residue was purified by silica gel column chromatography (1/1, petroleum ether/ethyl acetate, R_{f}=0.49) to obtain compound 8-2. MS-ESI calculated for [M+H]⁺ 568, found 568.

### Step 2

Compound 8-2 (213 mg, 375 μmol) was dissolved in ethyl acetate (5.00 mL), and hydrochloride ethyl acetate solution (4 mol/L, 1.88 mL) was added thereto, and the reaction mixture was stirred and reacted for 1 hour at 20° C. After the reaction mixture was concentrated, the residue was separated by preparative high performance liquid chromatography (chromatographic column: Venusil ASB Phenyl 150*30 mm*5 μm; mobile phase A: hydrochloric acid aqueous solution with a volume fraction of 0.05%; mobile phase B: acetonitrile; B (acetonitrile) %: 35% to 65%, 9 minutes) to obtain the hydrochloride of compound 8. ¹H NMR (400 MHz, CD₃OD) δ=7.72 (t, J=7.4 Hz, 1H), 7.65 (s, 1H), 7.34 (d, J=11.2 Hz, 1H), 7.28 (d, J=8.0 Hz, 1H), 7.19-7.03 (m, 3H), 4.22-4.05 (m, 1H), 4.05-3.92 (m, 2H), 3.92 (s, 3H), 3.82-3.70 (m, 1H), 3.67-3.56 (m, 1H), 2.29-2.10 (m, 1H), 2.08-1.90 (m, 2H), 1.90-1.78 (m, 1H), 1.73-1.48 (m, 2H). MS-ESI calculated for [M+H]⁺ 468, found 468.

### Example 9

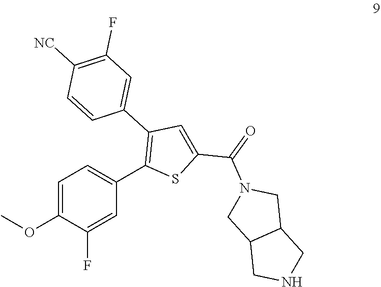 [SMILES: COc1ccc(-c2sc(C(=O)N3CC4CNCC4C3)cc2-c2ccc(C#N)c(F)c2)cc1F]

Synthetic Route:

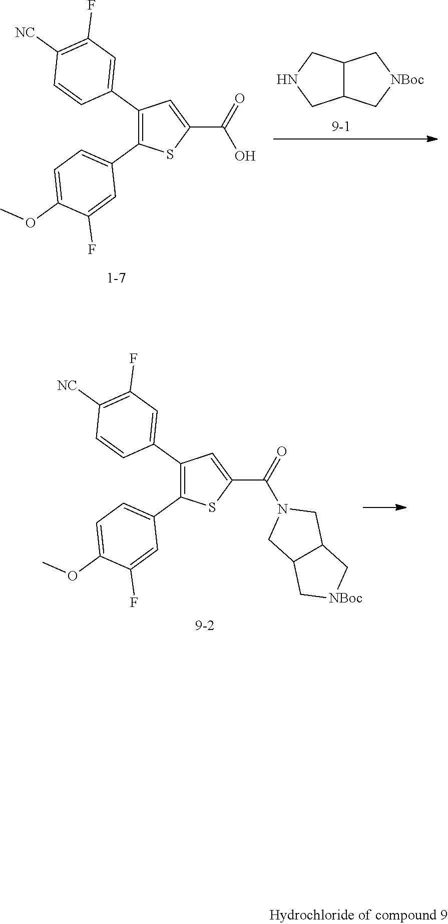 [SMILES: COc1ccc(-c2sc(C(=O)O)cc2-c2ccc(C#N)c(F)c2)cc1F ; CC(C)(C)OC(=O)N1CC2CNCC2C1 ; COc1ccc(-c2sc(C(=O)N3CC4CN(C(=O)OC(C)(C)C)CC4C3)cc2-c2ccc(C#N)c(F)c2)cc1F]

### Step 1

Compound 1-7 (150 mg, 404 μmol), 2-(7-azabenzotriazol-1-yl)-N,N,N′,N′-tetramethyluronium hexafluorophosphate (230 mg, 606 μmol), and N,N-diisopropylethylamine (157 mg, 1.21 mmol) were dissolved in N,N-dimethylformamide (5.00 mL), and then compound 9-1 (85.8 mg, 404 μmol) was added thereto, and the reaction mixture was stirred and reacted for 12 hours at 20° C. The reaction mixture was added with water (50 mL), and extracted with ethyl acetate (50 mL×2). The organic phase was washed with saturated brine (10 mL), dried over anhydrous sodium sulfate, filtered, and concentrated under reduced pressure. The residue was purified by silica gel column chromatography (1/1, petroleum ether/ethyl acetate, R_{f}=0.49) to obtain compound 9-2. MS-ESI calculated for [M−56+H]⁺ 510, found 510.

### Step 2

Compound 9-2 (280 mg, 495.02 μmol) was dissolved in ethyl acetate (5 mL), and hydrochloride ethyl acetate solution (4 mol/L, 2.48 mL) was added thereto, and the reaction mixture was stirred and reacted for 1 hour at 20° C. After the reaction mixture was concentrated, the residue was separated by preparative high performance liquid chromatography (chromatographic column: Venusil ASB Phenyl 150*30 mm*5 μm; mobile phase A: hydrochloric acid aqueous solution with a volume fraction of 0.05%; mobile phase B: acetonitrile B %: 35% to 65%, 9 minutes) to obtain the hydrochloride of compound 9. ¹H NMR (400 MHz, CD₃OD) δ=7.74-7.65 (m, 2H), 7.33 (d, J=10.4 Hz, 1H), 7.25 (d, J=1.3, 8.0 Hz, 1H), 7.15-7.00 (m, 3H), 3.90 (s, 3H), 3.62-3.58 (m, 2H), 3.31-3.26 (m, 8H). MS-ESI calculated for [M+H]⁺ 466, found 466.

### Example 10

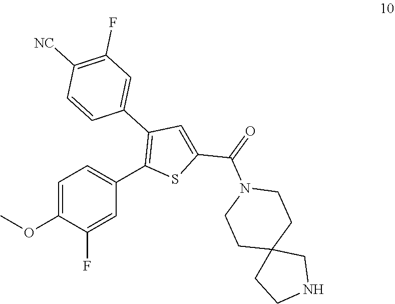 [SMILES: COc1ccc(-c2sc(C(=O)N3CCC4(CCNC4)CC3)cc2-c2ccc(C#N)c(F)c2)cc1F]

Synthetic Route:

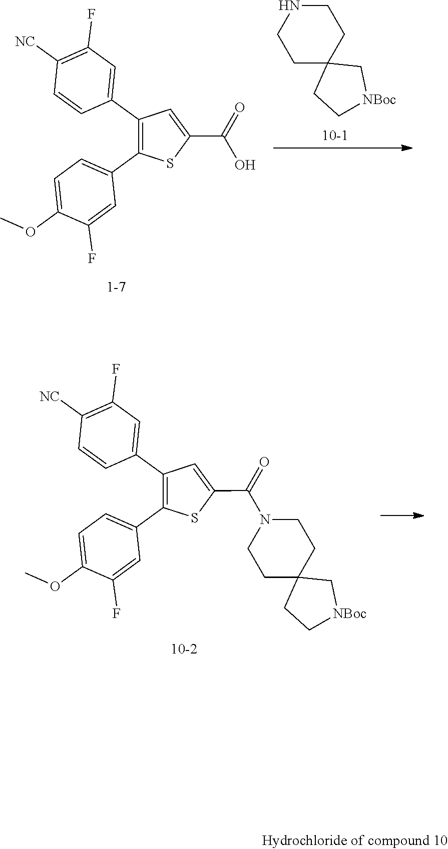 [SMILES: COc1ccc(-c2sc(C(=O)O)cc2-c2ccc(C#N)c(F)c2)cc1F ; CC(C)(C)OC(=O)N1CCC2(CCNCC2)C1 ; COc1ccc(-c2sc(C(=O)N3CCC4(CCN(C(=O)OC(C)(C)C)C4)CC3)cc2-c2ccc(C#N)c(F)c2)cc1F]

### Step 1

Compound 1-7 (150 mg, 404 μmol), 2-(7-azabenzotriazol-1-yl)-N,N,N′,N′-tetramethyluronium hexafluorophosphate (230 mg, 606 μmol), and N,N-diisopropylethylamine (157 mg, 1.21 mmol) were dissolved in N,N-dimethylformamide (5.00 mL), and then compound 10-1 (97.1 mg, 404 μmol) was added thereto, and the reaction mixture was stirred and reacted for 12 hours at 20° C. The reaction mixture was added with water (50 mL), and extracted with ethyl acetate (50 mL×2). The organic phase was washed with saturated brine (10 mL), dried over anhydrous sodium sulfate, filtered, and concentrated under reduced pressure. The residue was purified by silica gel column chromatography (1/1, petroleum ether/ethyl acetate, R_{f}=0.49) to obtain compound 10-2. MS-ESI calculated for [M−56+H]⁺ 538, found 538.

### Step 2

Compound 10-2 (300 mg, 505 μmol) was dissolved in ethyl acetate (5 mL), and hydrochloride ethyl acetate solution (4 mol/L, 2.53 mL) was added thereto, and the reaction mixture was stirred and reacted for 1 hour at 20° C. After the reaction mixture was concentrated, the residue was separated by preparative high performance liquid chromatography (chromatographic column: Venusil ASB Phenyl 150*30 mm*5 μm; mobile phase A: hydrochloric acid aqueous solution with a volume fraction of 0.05%; mobile phase B: acetonitrile B %: 35% to 65%, 9 minutes) to obtain the hydrochloride of compound 10. ¹H NMR (400 MHz, CD₃OD) δ=7.51 (s, 1H), 7.31 (d, J=10.4 Hz, 1H), 7.25 (d, J=8.0 Hz, 1H), 7.16-6.97 (m, 3H), 3.96-3.90 (m, 2H), 3.90 (s, 3H), 3.85-3.67 (m, 2H), 3.43 (t, J=7.2 Hz, 2H), 3.25-3.15 (m, 2H), 2.05 (t, J=7.6 Hz, 2H), 1.84-1.67 (m, 4H). MS-ESI calculated for [M+H]⁺ 494, found 494.

### Example 11

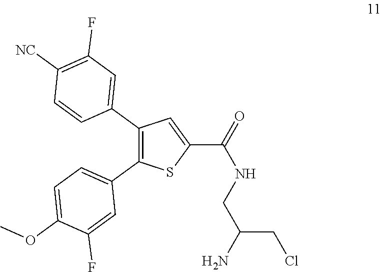 [SMILES: COc1ccc(-c2sc(C(=O)NCC(N)CCl)cc2-c2ccc(C#N)c(F)c2)cc1F]

Synthetic Route:

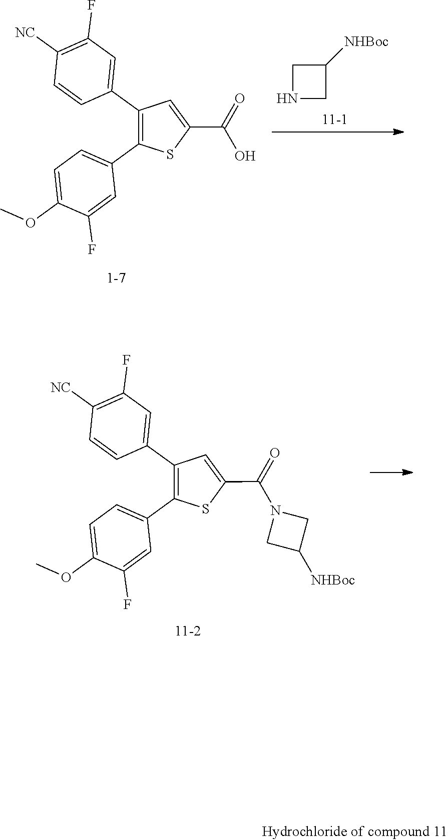 [SMILES: COc1ccc(-c2sc(C(=O)O)cc2-c2ccc(C#N)c(F)c2)cc1F ; CC(C)(C)OC(=O)NC1CNC1 ; COc1ccc(-c2sc(C(=O)N3CC(NC(=O)OC(C)(C)C)C3)cc2-c2ccc(C#N)c(F)c2)cc1F]

### Step 1

Compound 1-7 (150 mg, 404 μmol), 2-(7-azabenzotriazol-1-yl)-N,N,N′,N′-tetramethyluronium hexafluorophosphate (230 mg, 606 μmol), and N,N-diisopropylethylamine (156.61 mg, 1.21 mmol) were dissolved in N,N-dimethylformamide (5 mL), and then compound 11-1 (69.57 mg, 403.92 μmol) was added thereto, and the reaction mixture was stirred and reacted for 12 hours at 20° C. The reaction mixture was added with water (50 mL), and extracted with ethyl acetate (50 mL×2). The organic phase was washed with saturated brine (10 mL), dried over anhydrous sodium sulfate, filtered, and concentrated under reduced pressure. The residue was purified by silica gel column chromatography (1/1, petroleum ether/ethyl acetate, R_{f}=0.49) to obtain compound 11-2. Compound 11-2 MS-ESI calculated for [M+H]⁺ 526, found 526.

### Step 2

Compound 11-2 (300 mg, 571 μmol) was dissolved in ethyl acetate (5.00 mL), and hydrochloride ethyl acetate solution (4 mol/L, 2.85 mL) was added thereto, and the reaction mixture was stirred and reacted for 1 hour at 20° C. After the reaction mixture was concentrated, the residue was separated by preparative high performance liquid chromatography (chromatographic column: Venusil ASB Phenyl 150*30 mm*5 μm; mobile phase A: hydrochloric acid aqueous solution with a volume fraction of 0.05%; mobile phase B: acetonitrile B %: 35% to 65%, 9 minutes) to obtain the hydrochloride of compound 11. ¹H NMR (400 MHz, CD₃OD) δ=7.86 (s, 1H), 7.72 (t, J=7.4 Hz, 1H), 7.35-7.22 (m, 2H), 7.17-7.01 (m, 3H), 3.98-3.93 (m, 1H), 3.90 (s, 3H), 3.89-3.77 (m, 2H), 3.77-3.66 (m, 2H). MS-ESI calculated for [M+H]⁺ 462, found 462.

### Example 12

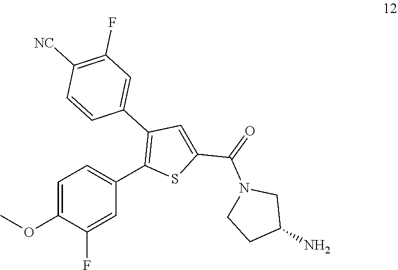 [SMILES: COc1ccc(-c2sc(C(=O)N3CC[C@@H](N)C3)cc2-c2ccc(C#N)c(F)c2)cc1F]

Synthetic Route:

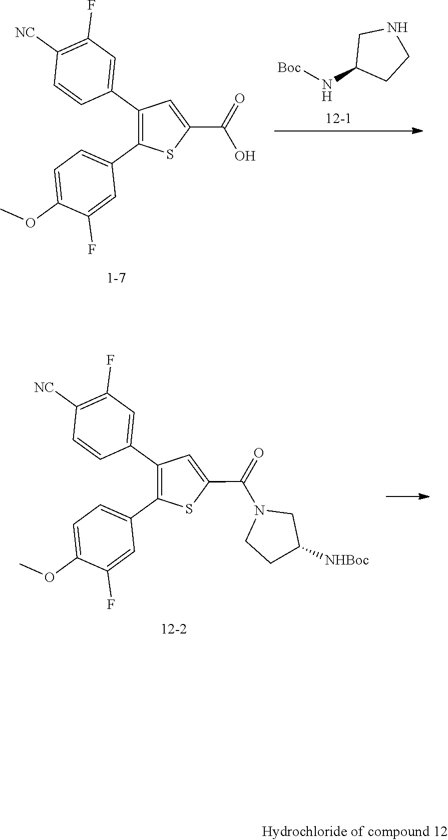 [SMILES: COc1ccc(-c2sc(C(=O)O)cc2-c2ccc(C#N)c(F)c2)cc1F ; CC(C)(C)OC(=O)N[C@@H]1CCNC1 ; COc1ccc(-c2sc(C(=O)N3CC[C@@H](NC(=O)OC(C)(C)C)C3)cc2-c2ccc(C#N)c(F)c2)cc1F]

### Step 1

Compound 1-7 (200 mg, 539 μmol), 2-(7-azabenzotriazol-1-yl)-N,N,N′,N′-tetramethyluronium hexafluorophosphate (307.17 mg, 808 μmol), and N,N-diisopropylethylamine (209 mg, 1.62 mmol) were dissolved in N,N-dimethylformamide (5.00 mL), and then compound 12-1 (100 mg, 539 μmol) was added thereto, and the reaction mixture was stirred and reacted for 12 hours at 20° C. The reaction mixture was added with water (50 mL), and extracted with ethyl acetate (50 mL×2). The organic phase was washed with saturated brine (10 mL), dried over anhydrous sodium sulfate, filtered, and concentrated under reduced pressure. The residue was purified by silica gel column chromatography (1/1, petroleum ether/ethyl acetate, R_{f}=0.57) to obtain compound 12-2. MS-ESI calculated for [M−56+H]⁺ 484, found 484.

### Step 2

Compound 12-2 (300 mg, 556 μmol) was dissolved in ethyl acetate (10.0 mL), and hydrochloride ethyl acetate solution (4 mol/L, 10.0 mL) was added thereto, and the reaction mixture was stirred and reacted for 12 hours at 20° C. After the reaction mixture was concentrated, the residue was separated by preparative high performance liquid chromatography (chromatographic column: Venusil ASB Phenyl 150*30 mm*5 μm; mobile phase A: hydrochloric acid aqueous solution with a volume fraction of 0.05%; mobile phase B: acetonitrile B %: 30% to 60%, 9 minutes) to obtain the hydrochloride of compound 12. ¹H NMR (400 MHz, CD₃OD) δ=7.77-7.66 (m, 2H), 7.32 (dd, J=1.2, 10.0 Hz, 1H), 7.26 (dd, J=1.2, 8.0 Hz, 1H), 7.16-7.02 (m, 3H), 4.40-3.92 (m, 4H), 3.90 (s, 3H), 3.85-3.81 (m, 1H), 2.60-2.40 (m, 1H), 2.35-2.10 (m, 1H). MS-ESI calculated for [M+H]⁺ 440, found 440.

### Example 13

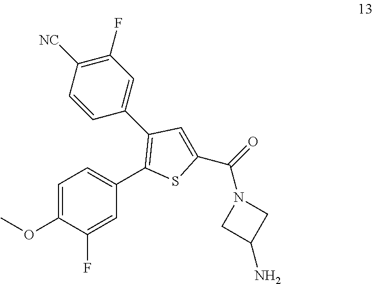 [SMILES: COc1ccc(-c2sc(C(=O)N3CC(N)C3)cc2-c2ccc(C#N)c(F)c2)cc1F]

Synthetic Route:

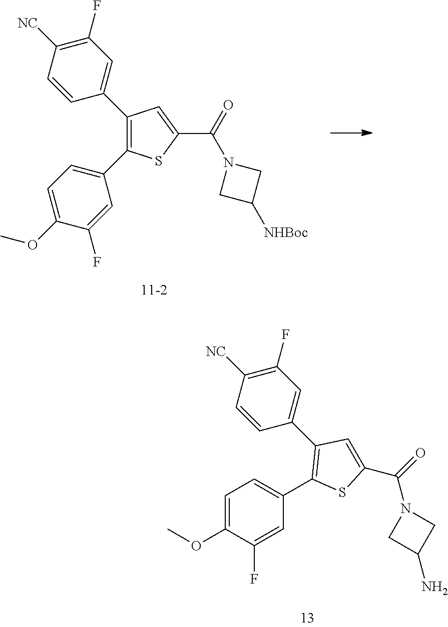 [SMILES: COc1ccc(-c2sc(C(=O)N3CC(NC(=O)OC(C)(C)C)C3)cc2-c2ccc(C#N)c(F)c2)cc1F ; COc1ccc(-c2sc(C(=O)N3CC(N)C3)cc2-c2ccc(C#N)c(F)c2)cc1F]

Compound 11-2 (270 mg, 514 μmol) was dissolved in dichloromethane (10.0 mL). Trimethylsilyl trifluoromethanesulfonate (120 mg, 539 μmol) was added thereto, and the reaction mixture was stirred and reacted at 20° C. for 12 hours. After the reaction mixture was concentrated, the residue was separated by preparative high performance liquid chromatography (chromatographic column: Phenomenex Gemini-NX 80*30 mm*3 μm; mobile phase A: 10 mM ammonium bicarbonate aqueous solution; mobile phase B: acetonitrile B %: 42% to 72%, 9 minutes) to obtain compound 13. Compound 13 ¹H NMR (400 MHz, CD₃OD) δ=7.69 (t, J=8.0 Hz, 1H), 7.59 (s, 1H), 7.29 (d, J=10.0 Hz, 1H), 7.23 (dd, J=1.4, 8.2 Hz, 1H), 7.14-6.98 (m, 3H), 4.79 (t, J=8.4 Hz, 1H), 4.69-4.54 (m, 1H), 4.46-4.34 (m, 1H), 4.30-4.18 (m, 1H), 3.98-3.92 (m, 1H), 3.89 (s, 3H). MS-ESI calculated for [M+H]⁺ 426, found 426.

### Example 14

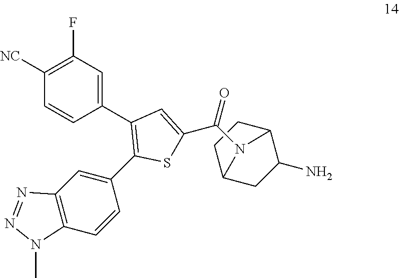 [SMILES: Cn1nnc2cc(-c3sc(C(=O)N4C5CCC4C(N)C5)cc3-c3ccc(C#N)c(F)c3)ccc21]

Synthetic Route:

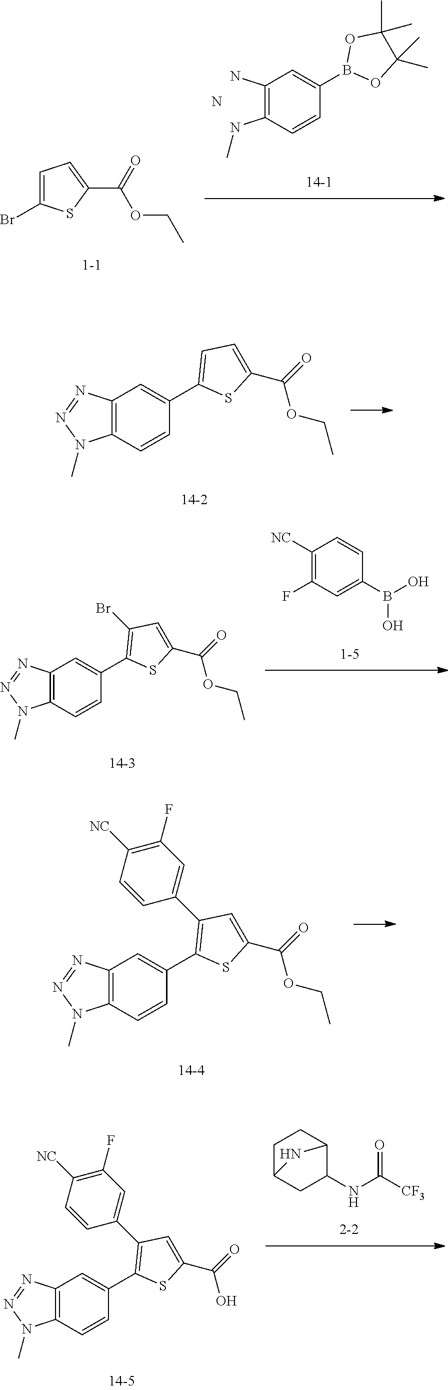

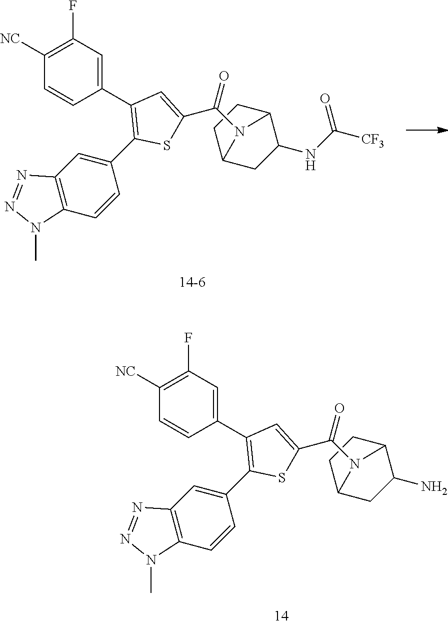 [SMILES: Cn1nnc2cc(-c3sc(C(=O)N4C5CCC4C(NC(=O)C(F)(F)F)C5)cc3-c3ccc(C#N)c(F)c3)ccc21 ; Cn1nnc2cc(-c3sc(C(=O)N4C5CCC4C(N)C5)cc3-c3ccc(C#N)c(F)c3)ccc21]

### Step 1

Under nitrogen atmosphere, compound 1-1 (992 mg, 4.22 mmol) and compound 14-1 (729 mg, 2.81 mmol) were dissolved in dioxane (10.0 mL) and water (2.00 mL), and then potassium carbonate (778 mg, 5.63 mmol) and tetrakis(triphenylphosphine)palladium (325 mg, 281 μmol) were added thereto. The reaction mixture was stirred and reacted for 12 hours at 80° C. The reaction mixture was added with water (50 mL), and extracted with ethyl acetate (50 mL×2). The organic phase was washed with saturated brine (10 mL), dried over anhydrous sodium sulfate, filtered, and concentrated under reduced pressure. The residue was purified by silica gel column chromatography (1/1, petroleum ether/ethyl acetate, R_{f}=0.3) to obtain compound 14-2. MS-ESI calculated for [M+H]⁺ 288, found 288.

### Step 2

Compound 14-2 (500 mg, 1.74 mmol) was dissolved in N,N-dimethylformamide (5.00 mL). N-Bromosuccinimide (619 mg, 3.48 mmol) was added thereto, and the reaction mixture was stirred and reacted for 12 hours at 25° C. The reaction mixture was concentrated under reduced pressure, and the residue was purified by silica gel column chromatography (1/1, petroleum ether/ethyl acetate, R_{f}=0.55) to obtain compound 14-3. MS-ESI calculated for [M+H]⁺ 366, found 366.

### Step 3

Under nitrogen atmosphere, compound 14-3 (730 mg, 1.99 mmol) and compound 1-5 (329 mg, 1.99 mmol) were dissolved in ethanol (10.0 mL) and water (2.00 mL), and then sodium carbonate (423 mg, 3.99 mmol) and bis(di-tert-butyl(4-dimethylaminophenyl)phosphine)dichloropalladium(II) (72.9 mg, 99.7 μmol) were added thereto. The reaction mixture was stirred and reacted for 12 hours at 80° C. After the reaction mixture was concentrated to remove the ethanol, the reaction mixture was added with water (50 mL), and extracted with ethyl acetate (50 mL×2). The organic phase was washed with saturated brine (10 mL), dried over anhydrous sodium sulfate, filtered, and concentrated under reduced pressure. The residue was purified by silica gel column chromatography (1/1, petroleum ether/ethyl acetate, R_{f}=0.2) to obtain compound 14-4. ¹H NMR (400 MHz, CD₃Cl) δ=8.08 (s, 1H), 7.89-7.78 (m, 1H), 7.63-7.42 (m, 2H), 7.33 (dd, J=1.4, 8.6 Hz, 1H), 7.22-7.01 (m, 2H), 4.47-4.38 (m, 2H), 4.34 (s, 3H), 1.46-1.39 (m, 3H). MS-ESI calculated for [M+H]⁺ 407, found 407.

### Step 4

Compound 14-4 (271 mg, 667 μmol) was dissolved in tetrahydrofuran (3.00 mL) and water (3.00 mL), and lithium hydroxide monohydrate (83.9 mg, 2.00 mmol) was added thereto, and the reaction mixture was stirred and reacted for 12 hours at 20° C. After the reaction mixture was concentrated to remove the tetrahydrofuran, the residue was added with water (50 mL). The mixture was acidified to pH=6 with 1 M dilute hydrochloric acid, and extracted with ethyl acetate (50 mL×3). The organic phases were combined, washed with saturated brine (10 mL), dried over anhydrous sodium sulfate, filtered, and concentrated to obtain crude compound 14-5. MS-ESI calculated for [M−H]⁺ 379, found 379.

### Step 5

Compound 14-5 (200 mg, 529 μmol) was dissolved in N,N-dimethylformamide (5.00 mL). 2-(7-Azabenzotriazol-1-yl)-N,N,N′,N′-tetramethyluronium hexafluorophosphate (301 mg, 793 μmol) and N,N-diisopropylethylamine (342 mg, 2.64 mmol) were added thereto, and then compound 2-2 (93.6 mg, 450 μmol) was added thereto, and the reaction mixture was stirred and reacted for 4 hours at 20° C. The reaction mixture was added with water (50 mL), and extracted with ethyl acetate (50 mL×2). The organic phase was washed with saturated brine (10 mL), dried over anhydrous sodium sulfate, filtered, and concentrated under reduced pressure. The residue was purified by silica gel column chromatography (1/1, petroleum ether/ethyl acetate, R_{f}=0.51) to obtain compound 14-6. MS-ESI calculated for [M+H]⁺ 569, found 569.

### Step 6

Compound 14-6 (160 mg, 281 μmol) was dissolved in tetrahydrofuran (10.0 mL) and water (5.00 mL), then sodium hydroxide (33.8 mg, 844 μmol) was added thereto, and the reaction mixture was stirred and reacted for 12 hours at 50° C. The reaction mixture was added with water (50 mL), and extracted with ethyl acetate (50 mL×3). The organic phase was washed with saturated brine (10 mL), dried over anhydrous sodium sulfate, filtered, and concentrated under reduced pressure. The residue was purified by preparative high performance liquid chromatography (chromatographic column: Phenomenex Gemini-NX 80*30 mm*3 μm; mobile phase A: 10 mM ammonium bicarbonate aqueous solution; mobile phase B: acetonitrile B %: 33% to 63%, 9 minutes) to obtain compound 14. ¹H NMR (400 MHz, CDCl₃) δ=8.06 (s, 1H), 7.55 (s, 1H), 7.54-7.47 (m, 2H), 7.33 (dd, J=1.6, 8.8 Hz, 1H), 7.16-7.06 (m, 2H), 4.74-4.64 (m, 1H), 4.55 (s, 1H), 4.34 (s, 3H), 3.71-3.61 (m, 1H), 2.47-2.38 (m, 1H), 2.31-2.19 (m, 1H), 1.99-1.92 (m, 1H), 1.87-1.81 (m, 1H), 1.29-1.24 (m, 1H), 1.02-0.99 (m, 1H). MS-ESI calculated for [M+H]⁺ 473, found 473.

### Example 15

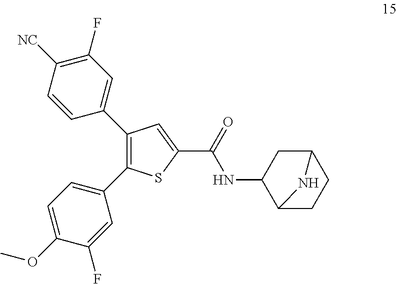 [SMILES: COc1ccc(-c2sc(C(=O)NC3CC4CCC3N4)cc2-c2ccc(C#N)c(F)c2)cc1F]

Synthetic Route:

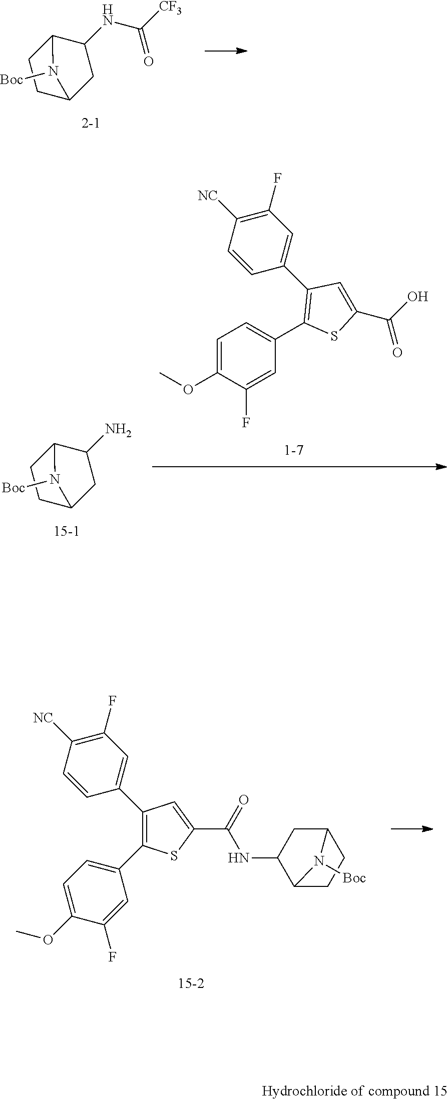 [SMILES: CC(C)(C)OC(=O)N1C2CCC1C(NC(=O)C(F)(F)F)C2 ; COc1ccc(-c2sc(C(=O)O)cc2-c2ccc(C#N)c(F)c2)cc1F ; CC(C)(C)OC(=O)N1C2CCC1C(N)C2 ; COc1ccc(-c2sc(C(=O)NC3CC4CCC3N4C(=O)OC(C)(C)C)cc2-c2ccc(C#N)c(F)c2)cc1F]

### Step 1

Compound 2-1 (100 mg, 324 μmol) was dissolved in tetrahydrofuran (5.00 mL) and water (1.00 mL), then sodium hydroxide (26.0 mg, 650 μmol) was added thereto, and the reaction mixture was stirred and reacted for 12 hours at 50° C. The reaction mixture was added with water (10 mL) and extracted with ethyl acetate (20 mL×2). The organic phase was washed with saturated brine (30 mL), dried over anhydrous sodium sulfate, filtered, and concentrated under reduced pressure to obtain compound 15-1. MS-ESI calculated for [M+H]⁺ 213, found 213.

### Step 2

Compound 1-7 (70.0 mg, 188 μmol) was dissolved in anhydrous N,N-dimethylformamide (3.00 mL). Compound 15-1 (44.0 mg, 207 μmol), 2-(7-azabenzotriazol-1-yl)-N,N,N′,N′-tetramethyluronium hexafluorophosphate (108 mg, 283 μmol), and N,N-diisopropylethylamine (97.5 mg, 754 μmol) were added thereto, and the reaction mixture was stirred for 12 hours at 25° C. The reaction mixture was added with water (30 mL) and filtered, and the filter cake was washed with water (20 mL) and dried under vacuum to obtain compound 15-2. MS-ESI calculated for [M+Na]⁺ 588, found 588.

### Step 3

Compound 15-2 (100 mg, 177 μmol) was dissolved in ethyl acetate (5.00 mL), and hydrochloride ethyl acetate solution (4 mol/L, 5 mL) was added thereto. The reaction mixture was stirred and reacted for 2 hours at 25° C. The reaction mixture was concentrated under reduced pressure, and the residue was purified by preparative high performance liquid chromatography (Phenomenex Gemini-NX C18 75*30 mm*3 μm; mobile phase A: hydrochloric acid aqueous solution with a volume fraction of 0.05%; mobile phase B: acetonitrile; B %: 29% to 49%, 7 minutes) to obtain the hydrochloride of compound 15. ¹H NMR (400 MHz, CD₃OD) δ=8.01 (s, 1H), 7.76-7.74 (m, 1H), 7.33-7.27 (m, 2H), 7.14-7.06 (m, 3H), 4.57-4.52 (m, 1H), 4.46 (t, J=4.4 Hz, 1H), 4.27 (t, J=4.8 Hz, 1H), 3.92 (s, 3H), 2.51-2.43 (m, 1H), 2.18-2.14 (m, 1H), 1.99-1.94 (m, 3H), 1.86-1.81 (m, 1H). MS-ESI calculated for [M+H]⁺ 466, found 466.

### Example 16

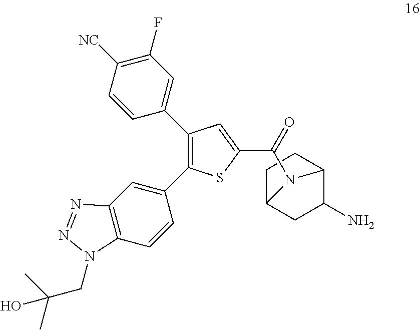 [SMILES: CC(C)(O)Cn1nnc2cc(-c3sc(C(=O)N4C5CCC4C(N)C5)cc3-c3ccc(C#N)c(F)c3)ccc21]

Synthetic Route:

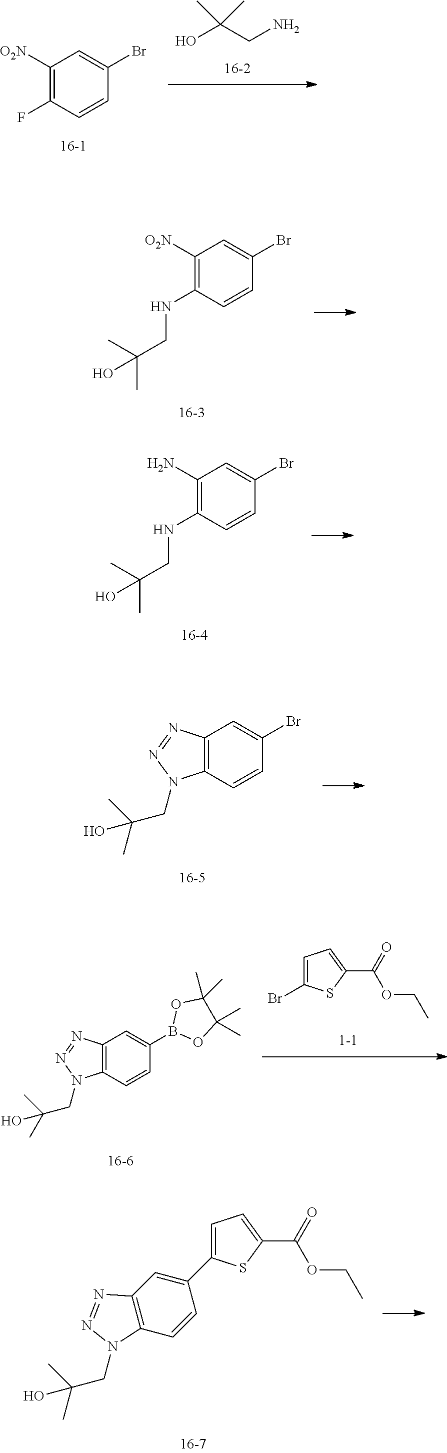

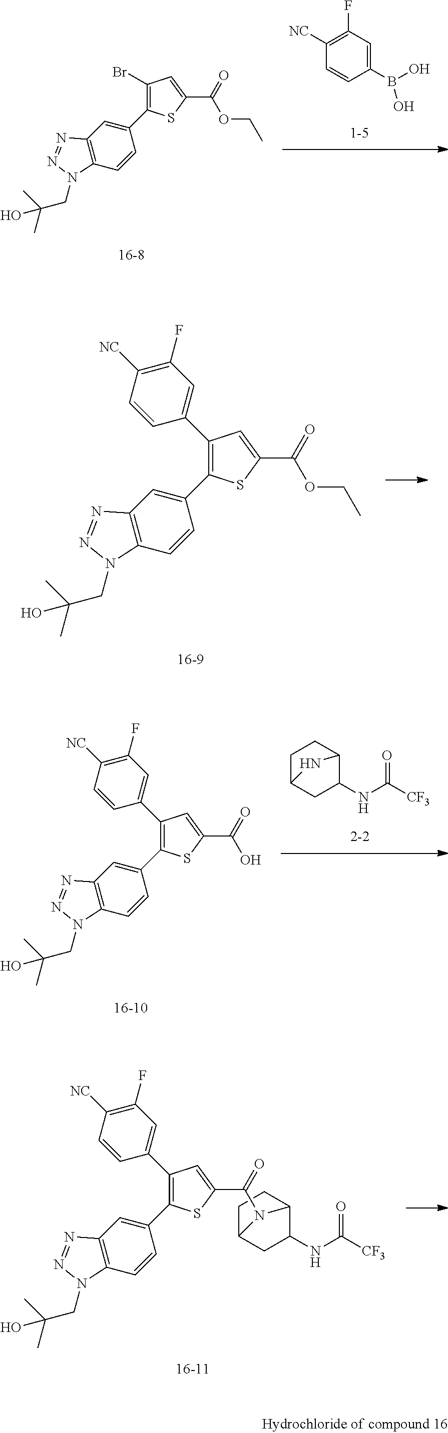 [SMILES: CCOC(=O)c1cc(Br)c(-c2ccc3c(c2)nnn3CC(C)(C)O)s1 ; N#Cc1ccc(B(O)O)cc1F ; CCOC(=O)c1cc(-c2ccc(C#N)c(F)c2)c(-c2ccc3c(c2)nnn3CC(C)(C)O)s1 ; CC(C)(O)Cn1nnc2cc(-c3sc(C(=O)O)cc3-c3ccc(C#N)c(F)c3)ccc21 ; O=C(NC1CC2CCC1N2)C(F)(F)F ; CC(C)(O)Cn1nnc2cc(-c3sc(C(=O)N4C5CCC4C(NC(=O)C(F)(F)F)C5)cc3-c3ccc(C#N)c(F)c3)ccc21]

### Step 1

Compound 16-1 (10.0 g, 45.5 mmol) was dissolved in N,N-dimethylformamide (120 mL), then 16-2 (4.86 g, 54.6 mmol) and N,N-diisopropylethylamine (11.8 g, 90.9 mmol) were added thereto, and the reaction mixture was stirred and reacted for 16 hours at 80° C. To the stirred water (600 mL) was added the reaction mixture. The resulting suspension was filtered, and the filter cake was washed with water (100 mL) and dried under vacuum to obtain compound 16-3. ¹H NMR (400 MHz, CDCl₃) δ=8.28 (s, 1H), 8.24 (d, J=2.8 Hz, 1H), 7.40 (dd, J=2.4, 9.2 Hz, 1H), 6.76 (d, J=9.2 Hz, 1H), 3.22 (d, J=5.6 Hz, 2H), 1.30 (s, 6H). MS-ESI calculated for [M+H]⁺ 289, found 289.

### Step 2

Compound 16-3 (3.00 g, 10.4 mmol) was dissolved in concentrated hydrochloric acid (30 mL), then tin dichloride dihydrate (14.1 g, 62.3 mmol) was added thereto, and the reaction mixture was stirred and reacted for 0.5 hours at 25° C. The pH of the reaction mixture was adjusted to 13 by adding sodium hydroxide (1 mol/L aqueous solution). The resulting suspension was filtered, and the collected filter cake was washed with water (50 mL) and dried under vacuum. The dried filter cake was purified by silica gel column chromatography (2/1, petroleum ether/ethyl acetate, R_{f}=0.3) to obtain compound 16-4. ¹H NMR (400 MHz, CDCl₃) δ=6.82-6.80 (m, 1H), 6.76 (d, J=2.4 Hz, 1H), 6.46 (d, J=8.4 Hz, 1H), 3.41-3.28 (m, 3H), 2.95 (s, 2H), 1.26 (s, 6H). MS-ESI calculated for [M+H]⁺ 261, found 261.

### Step 3

Compound 16-4 (1.41 g, 5.44 mmol) was dissolved in acetic acid (20 mL), and a solution of sodium nitrite (564 mg, 8.17 mmol) in water (10 mL) was added dropwise thereto, and the reaction mixture was stirred for 5 hours at 25° C. The reaction mixture was added with water (30 mL), and extracted with ethyl acetate (50 mL×2). The organic phase was sequentially washed with saturated sodium bicarbonate aqueous solution (100 mL) and saturated brine (100 mL), dried over anhydrous sodium sulfate, filtered, and concentrated under reduced pressure, and the residue was purified by silica gel column chromatography (1/1, petroleum ether/ethyl acetate, R_{f}=0.30) to obtain compound 16-5. ¹H NMR (400 MHz, CDCl₃) δ=8.11-8.10 (m, 1H), 7.50-7.49 (m, 2H), 4.52 (s, 2H), 1.25 (s, 6H). MS-ESI calculated for [M+H]⁺ 272, found 272.

### Step 4

Under nitrogen atmosphere, compound 16-5 (1.66 g, 5.77 mmol) was dissolved in 1,4-dioxane (30 mL), and then bis(pinacolato)diboron (1.61 g, 6.35 mmol), potassium acetate (1.14 g, 11.6 mmol), and [1,1′-bis(diphenylphosphino)ferrocene]dichloropalladium(II) (427 mg, 583 μmol) were added thereto. The reaction mixture was stirred for 12 hours at 90° C. The reaction mixture was filtered through diatomite, and the filtrate was concentrated under reduced pressure, and the residue was purified by silica gel column chromatography (1/1, petroleum ether/ethyl acetate, R_{f}=0.50) to obtain compound 16-6. ¹H NMR (400 MHz, CDCl₃) δ=8.50 (s, 1H), 7.84 (d, J=8.0 Hz, 1H), 7.56 (d, J=8.4 Hz, 1H), 4.55 (s, 2H), 1.30 (s, 12H), 1.23 (s, 6H). MS-ESI calculated for [M+H]⁺ 318, found 318.

### Step 5

Under nitrogen atmosphere, compound 1-1 (900 mg, 3.83 mmol) was dissolved in 1,4-dioxane (20 mL) and water (4 mL), and then 16-6 (1.28 g, 4.04 mmol), potassium phosphate (1.63 g, 7.66 mmol), and 1,1′-bis (di-tert-butylphosphino)ferrocene dichloropalladium(II) (250 mg, 383 μmol) were added thereto. The reaction mixture was stirred for 12 hours at 90° C. The reaction mixture was filtered through diatomite, and the filtrate was concentrated under reduced pressure, and the residue was purified by silica gel column chromatography (1/1, petroleum ether/ethyl acetate, R_{f}=0.20) to obtain compound 16-7. MS-ESI calculated for [M+H]⁺ 346, found 346.

### Step 6

Compound 16-7 (487 mg, 1.41 mmol) and N-bromosuccinimide (500 mg, 2.81 mmol) were dissolved in acetonitrile (10.0 mL), and the reaction mixture was stirred for 24 hours at 80° C. The reaction mixture was added with ethyl acetate (20 mL), and sequentially washed with water (20 mL×1) and saturated brine (20 mL×1). The organic phase was dried over anhydrous sodium sulfate, filtered, and the filtrate was concentrated under reduced pressure, and the residue was purified by silica gel column chromatography (1/1, petroleum ether/ethyl acetate, R_{f}=0.50) to obtain compound 16-8. ¹H NMR (400 MHz, CDCl₃) δ=8.39-8.38 (m, 1H), 7.80-7.77 (m, 3H), 4.67 (s, 2H), 4.43-4.38 (m, 2H), 1.44-1.40 (m, 3H), 1.37 (s, 6H). MS-ESI calculated for [M+H]⁺ 426, found 426.

### Step 7

Under nitrogen atmosphere, compound 16-8 (730 mg, 1.72 mmol) was dissolved in 1,4-dioxane (10.0 mL) and water (2.00 mL), and then compound 1-5 (300 mg, 1.82 mmol), potassium phosphate (730 mg, 3.44 mmol), and 1,1′-bis (di-tert-butylphosphino)ferrocene dichloropalladium(II) (112 mg, 172 μmol) were added thereto. The reaction mixture was stirred for 12 hours at 90° C. The reaction mixture was filtered through diatomite, and the filtrate was concentrated under reduced pressure, and the residue was purified by silica gel column chromatography (1/1, petroleum ether/ethyl acetate, R_{f}=0.43) to obtain compound 16-9. ¹H NMR (400 MHz, CDCl₃) δ=8.06 (s, 1H), 7.87 (s, 1H), 7.67-7.65 (m, 1H), 7.54-7.52 (m, 1H), 7.35-7.33 (m, 1H), 7.17-7.11 (m, 1H), 4.64 (s, 2H), 4.45-4.40 (m, 2H), 1.45-1.42 (m, 3H), 1.37 (s, 6H). MS-ESI calculated for [M+H]⁺ 465, found 465.

### Step 8

Compound 16-9 (150 mg, 296 μmol) was dissolved in tetrahydrofuran (5.00 mL) and water (1.00 mL), then lithium hydroxide monohydrate (26.0 mg, 619 μmol) was added thereto, and the reaction mixture was stirred and reacted for 12 hours at 28° C. The pH of the reaction mixture was adjusted to 4 by adding dilute hydrochloric acid (1 N), and the mixture was extracted with ethyl acetate (30 mL×2). The organic phase was washed with saturated brine (50 mL), dried over anhydrous sodium sulfate, filtered, and concentrated under reduced pressure to obtain compound 16-10. MS-ESI calculated for [M+H]⁺ 437, found 437.

### Step 9

Compound 16-10 (188 mg, 431 μmol) was dissolved in anhydrous N,N-dimethylformamide (6.00 mL), and then compound 2-2 (107 mg, 437 μmol), 2-(7-azabenzotriazol-1-yl)-N,N,N′,N′-tetramethyluronium hexafluorophosphate (246 mg, 646 μmol), and N,N-diisopropylethylamine (223 mg, 1.72 mmol) were added thereto, and the reaction mixture was stirred for 12 hours at 28° C. To water (30 mL) was added the reaction mixture. The resulting suspension was filtered, and the collected filter cake was washed with water (20 mL) and dried under vacuum to obtain compound 16-11. MS-ESI calculated for [M+Na]⁺ 627, found 627.

### Step 10

Compound 16-11 (83.0 mg, 132 μmol) was dissolved in methanol (3.00 mL) and water (3.00 mL), then potassium carbonate (36.6 mg, 265 μmol) was added thereto, and the reaction mixture was stirred for 12 hours at 28° C. The reaction mixture was extracted with ethyl acetate (30 mL×2), and the organic phase was washed with saturated brine (50 mL), dried over anhydrous sodium sulfate, filtered, and concentrated under reduced pressure. The residue was purified by preparative high performance liquid chromatography (Phenomenex Luna C18 75*30 mm*3 μm; mobile phase A: hydrochloric acid aqueous solution with a volume fraction of 0.05%; mobile phase B: acetonitrile; B %: 18% to 38%, 6.5 minutes) to obtain the hydrochloride of compound 16. ¹H NMR (400 MHz, CD₃OD) δ=7.97 (s, 1H), 7.87-7.81 (m, 2H), 7.67-7.64 (m, 1H), 7.42-7.34 (m, 2H), 7.28-7.19 (m, 1H), 4.96-4.93 (m, 2H), 4.69 (s, 2H), 3.89-3.87 (m, 1H), 2.56-2.52 (m, 1H), 2.06-1.97 (m, 3H), 1.85-1.83 (m, 1H), 1.56-1.54 (m, 1H), 1.29 (s, 6H). MS-ESI calculated for [M+H]⁺ 531, found 531.

### Example 17

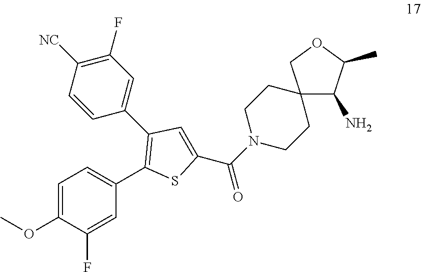

Synthetic Route:

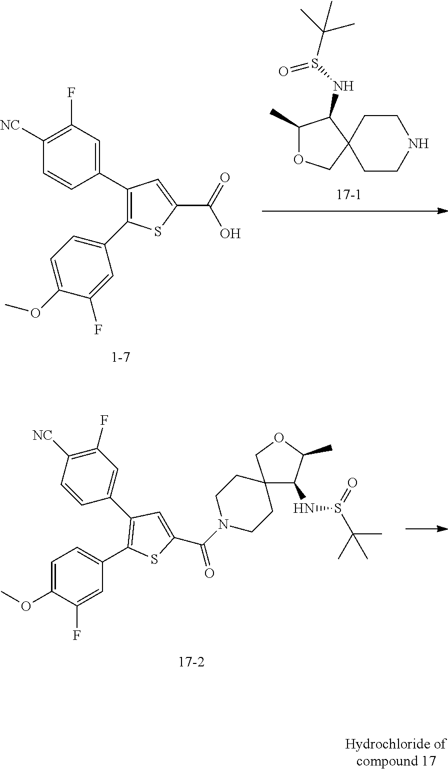

### Step 1

Compound 1-7 (217 mg, 583 μmol), 2-(7-azabenzotriazol-1-yl)-N,N,N′,N′-tetramethyluronium hexafluorophosphate (332 mg, 875 μmol), and N,N-diisopropylethylamine (226 mg, 1.75 mmol) were dissolved in N,N-dimethylformamide (5.00 mL), and then compound 17-1 (160 mg, 583 μmol) was added thereto, and the reaction mixture was stirred and reacted for 12 hours at 25° C. The reaction mixture was added with water (100 mL), and extracted with ethyl acetate (20 mL×3). The organic phase was washed with saturated brine (100 mL), dried over anhydrous sodium sulfate, filtered, and concentrated under reduced pressure. The residue was purified by silica gel column chromatography (1/1, petroleum ether/ethyl acetate, R_{f}=0.14) to obtain compound 17-2. MS-ESI calculated for [M+H]⁺ 628, found 628.

### Step 2

Compound 17-2 (250 mg, 398 μmol) was dissolved in ethyl acetate (3.00 mL), and hydrochloride ethyl acetate solution (4 mol/L, 5.00 mL) was added thereto, and the reaction mixture was stirred and reacted for 5 hours at 25° C. After the reaction mixture was concentrated, the residue was separated by preparative high performance liquid chromatography (chromatographic column: Venusil ASB Phenyl 150*30 mm*5 μm; mobile phase A: hydrochloric acid aqueous solution with a volume fraction of 0.05%; mobile phase B: acetonitrile B %: 35% to 65%, 9 minutes) to obtain the hydrochloride of compound 17. ¹H NMR (400 MHz, CD₃OD) δ=7.70 (t, J=7.6 Hz, 1H), 7.52 (s, 1H), 7.32 (d, J=10.4 Hz, 1H), 7.25 (d, J=8.4 Hz, 1H), 7.15-7.03 (m, 3H), 4.90-4.89 (m, 1H), 4.39-4.28 (m, 3H), 4.01-3.99 (m, 1H), 3.90-3.83 (m, 4H), 3.47-3.46 (m, 1H), 1.97-1.66 (m, 4H), 1.32 (d, J=6.6 Hz, 3H). MS-ESI calculated for [M+H]⁺ 524, found 524.

### Example 18

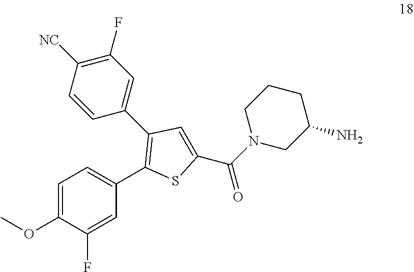 [SMILES: COc1ccc(-c2sc(C(=O)N3CCC[C@H](N)C3)cc2-c2ccc(C#N)c(F)c2)cc1F]

Synthetic Route:

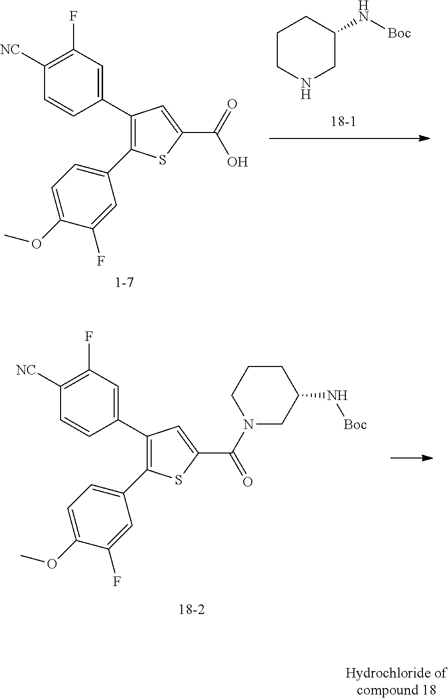 [SMILES: COc1ccc(-c2sc(C(=O)O)cc2-c2ccc(C#N)c(F)c2)cc1F ; CC(C)(C)OC(=O)N[C@H]1CCCNC1 ; COc1ccc(-c2sc(C(=O)N3CCC[C@H](NC(=O)OC(C)(C)C)C3)cc2-c2ccc(C#N)c(F)c2)cc1F]

### Step 1

Compound 1-7 (200 mg, 539 μmol), 2-(7-azabenzotriazol-1-yl)-N,N,N′,N′-tetramethyluronium hexafluorophosphate (307 mg, 808 μmol), and N,N-diisopropylethylamine (209 mg, 1.62 mmol) were dissolved in N,N-dimethylformamide (3.00 mL), and then compound 18-1 (108 mg, 539 μmol) was added thereto, and the reaction mixture was stirred and reacted for 12 hours at 25° C. The reaction mixture was added with water (50 mL), and extracted with ethyl acetate (20 mL×3). The organic phase was washed with saturated brine (50 mL), dried over anhydrous sodium sulfate, filtered, and concentrated under reduced pressure. The residue was purified by silica gel column chromatography (1/1, petroleum ether/ethyl acetate, R_{f}=0.41) to obtain compound 18-2. MS-ESI calculated for [M−56+H]⁺ 498, found 498.

### Step 2

Compound 18-2 (265 mg, 479 μmol) was dissolved in ethyl acetate (5 mL), and hydrochloride ethyl acetate solution (4 mol/L, 5.00 mL) was added thereto, and the reaction mixture was stirred and reacted for 2 hours at 25° C. After the reaction mixture was concentrated, the residue was separated by preparative high performance liquid chromatography (chromatographic column: Phenomenex Synergi C18 150*30 mm*4 μm; mobile phase A: hydrochloric acid aqueous solution with a volume fraction of 0.05%; mobile phase B: acetonitrile B %: 34% to 64%, 9 minutes) to obtain the hydrochloride of compound 18. ¹H NMR (400 MHz, CD₃OD) δ=7.69 (t, J=7.4 Hz, 1H), 7.56 (s, 1H), 7.33 (d, J=10.4 Hz, 1H), 7.26 (d, J=8.0 Hz, 1H), 7.15-7.01 (m, 3H), 4.46-4.32 (m, 1H), 4.21-4.09 (m, 1H), 3.90 (s, 3H), 3.59-3.36 (m, 3H), 2.25-2.15 (m, 1H), 1.97-1.64 (m, 3H). MS-ESI calculated for [M+H]⁺ 454, found 454.

### Example 19

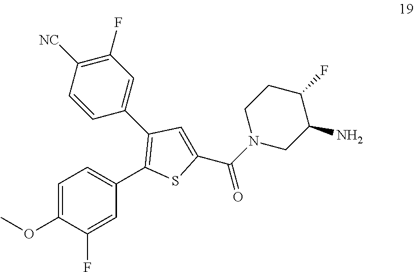 [SMILES: COc1ccc(-c2sc(C(=O)N3CC[C@H](F)[C@@H](N)C3)cc2-c2ccc(C#N)c(F)c2)cc1F]

Synthetic Route:

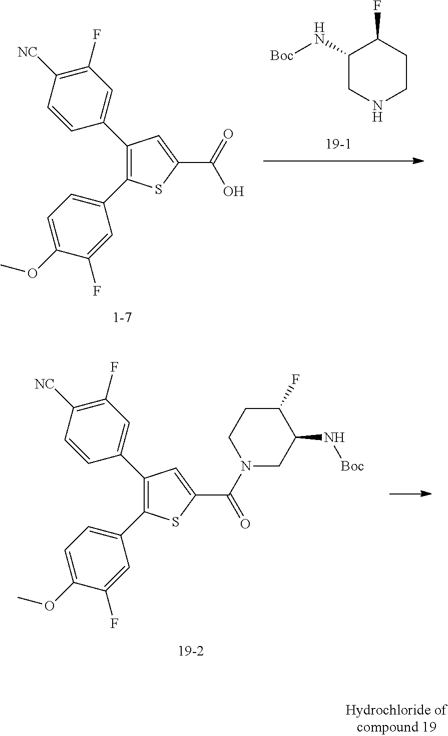 [SMILES: COc1ccc(-c2sc(C(=O)O)cc2-c2ccc(C#N)c(F)c2)cc1F ; CC(C)(C)OC(=O)N[C@H]1CNCC[C@@H]1F ; COc1ccc(-c2sc(C(=O)N3CC[C@H](F)[C@@H](NC(=O)OC(C)(C)C)C3)cc2-c2ccc(C#N)c(F)c2)cc1F]

### Step 1

Compound 1-7 (180 mg, 485 μmol), 2-(7-azabenzotriazol-1-yl)-N,N,N′,N′-tetramethyluronium hexafluorophosphate (276 mg, 727 μmol), and N,N-diisopropylethylamine (188 mg, 1.45 mmol) were dissolved in N,N-dimethylformamide (3.00 mL), and then compound 19-1 (116 mg, 533 μmol) was added thereto, and the reaction mixture was stirred and reacted for 12 hours at 25° C. The reaction mixture was added with water (50 mL), and extracted with ethyl acetate (20 mL×3). The organic phase was washed with saturated brine (50 mL), dried over anhydrous sodium sulfate, filtered, and concentrated under reduced pressure. The residue was purified by silica gel column chromatography (1/1, petroleum ether/ethyl acetate, R_{f}=0.50) to obtain compound 19-2. MS-ESI calculated for [M−56+H]⁺ 516, found 516.

### Step 2

Compound 19-2 (180 mg, 315 μmol) was dissolved in ethyl acetate (10.0 mL), and hydrochloride ethyl acetate solution (4 mol/L, 4.50 mL) was added thereto, and the reaction mixture was stirred and reacted for 6 hours at 25° C. After the reaction mixture was concentrated, the residue was separated by preparative high performance liquid chromatography (chromatographic column: Xtimate C18 150*40 mm*5 μm; mobile phase A: hydrochloric acid aqueous solution with a volume fraction of 0.05%; mobile phase B: acetonitrile B %: 16% to 46%, 10 minutes) to obtain the hydrochloride of compound 19. ¹H NMR (400 MHz, CD₃OD) δ=7.73-7.66 (m, 1H), 7.61 (s, 1H), 7.35 (d, J=10.4 Hz, 1H), 7.27 (d, J=8.0 Hz, 1H), 7.15-7.02 (m, 3H), 4.83-4.68 (m, 2H), 4.52-4.47 (m, 1H), 3.90 (s, 3H), 3.61-3.45 (m, 1H), 3.43-3.34 (m, 1H), 3.29-3.21 (m, 1H), 2.38-2.26 (m, 1H), 2.00-1.82 (m, 1H). MS-ESI calculated for [M+H]⁺ 472, found 472.

### Example 20

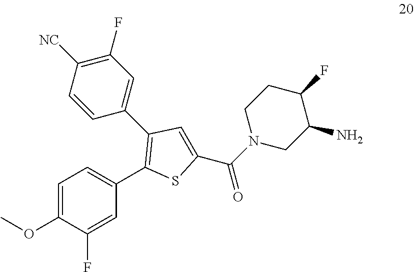 [SMILES: COc1ccc(-c2sc(C(=O)N3CC[C@@H](F)[C@@H](N)C3)cc2-c2ccc(C#N)c(F)c2)cc1F]

Synthetic Route:

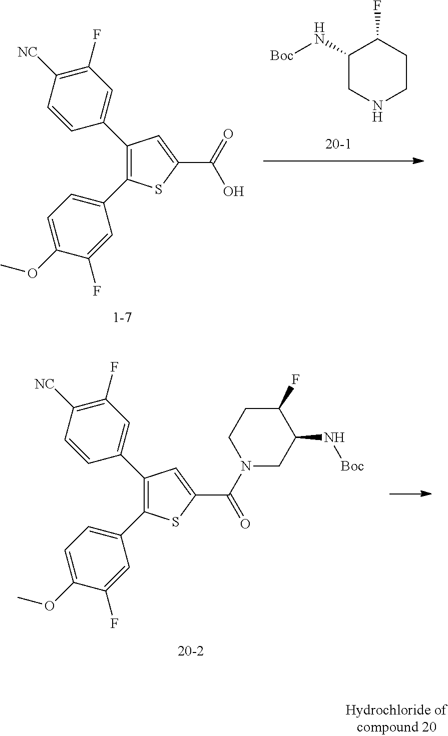 [SMILES: COc1ccc(-c2sc(C(=O)O)cc2-c2ccc(C#N)c(F)c2)cc1F ; CC(C)(C)OC(=O)N[C@H]1CNCC[C@H]1F ; COc1ccc(-c2sc(C(=O)N3CC[C@@H](F)[C@@H](NC(=O)OC(C)(C)C)C3)cc2-c2ccc(C#N)c(F)c2)cc1F]

### Step 1

Compound 1-7 (180 mg, 485 μmol), 2-(7-azabenzotriazol-1-yl)-N,N,N′,N′-tetramethyluronium hexafluorophosphate (276 mg, 727 μmol), and N,N-diisopropylethylamine (188 mg, 1.45 mmol) were dissolved in N,N-dimethylformamide (3.00 mL), and then compound 20-1 (116 mg, 533 μmol) was added thereto, and the reaction mixture was stirred and reacted for 12 hours at 25° C. The reaction mixture was added with water (50 mL), and extracted with ethyl acetate (20 mL×3). The organic phase was washed with saturated brine (50 mL), dried over anhydrous sodium sulfate, filtered, and concentrated under reduced pressure. The residue was purified by silica gel column chromatography (1/1, petroleum ether/ethyl acetate, R_{f}=0.55) to obtain compound 20-2. MS-ESI calculated for [M−56+H]⁺ 516, found 516.

### Step 2

Compound 20-2 (140 mg, 245 μmol) was dissolved in ethyl acetate (10.0 mL), and hydrochloride ethyl acetate solution (4 mol/L, 3.50 mL) was added thereto, and the reaction mixture was stirred and reacted for 6 hours at 25° C. After the reaction mixture was concentrated, the residue was separated by preparative high performance liquid chromatography (chromatographic column: Xtimate C18 150*40 mm*5 μm; mobile phase A: hydrochloric acid aqueous solution with a volume fraction of 0.05%; mobile phase B: acetonitrile B %: 16% to 46%, 10 minutes) to obtain the hydrochloride of compound 20. ¹H NMR (400 MHz, CD₃OD) δ=7.72-7.68 (m, 1H), 7.60 (s, 1H), 7.34 (d, J=10.4 Hz, 1H), 7.26 (d, J=8.0 Hz, 1H), 7.15-7.03 (m, 3H), 5.27-5.06 (m, 1H), 4.49-4.44 (m, 1H), 4.26-4.22 (m, 1H), 3.90 (s, 3H), 3.78-3.53 (m, 3H), 2.28-1.96 (m, 2H). MS-ESI calculated for [M+H]⁺ 472, found 472.

### Example 21

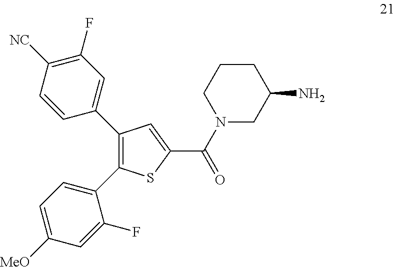 [SMILES: COc1ccc(-c2sc(C(=O)N3CCC[C@@H](N)C3)cc2-c2ccc(C#N)c(F)c2)c(F)c1]

Synthetic Route:

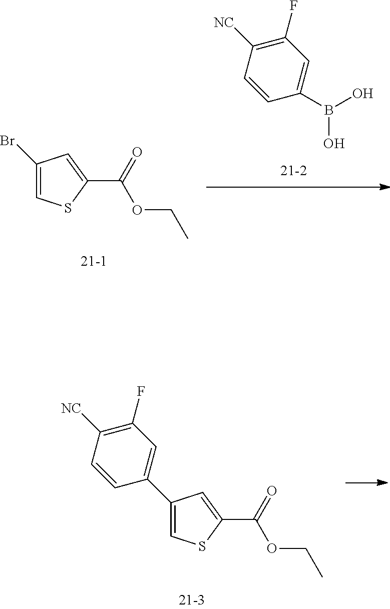 [SMILES: CCOC(=O)c1cc(Br)cs1 ; N#Cc1ccc(B(O)O)cc1F ; CCOC(=O)c1cc(-c2ccc(C#N)c(F)c2)cs1]

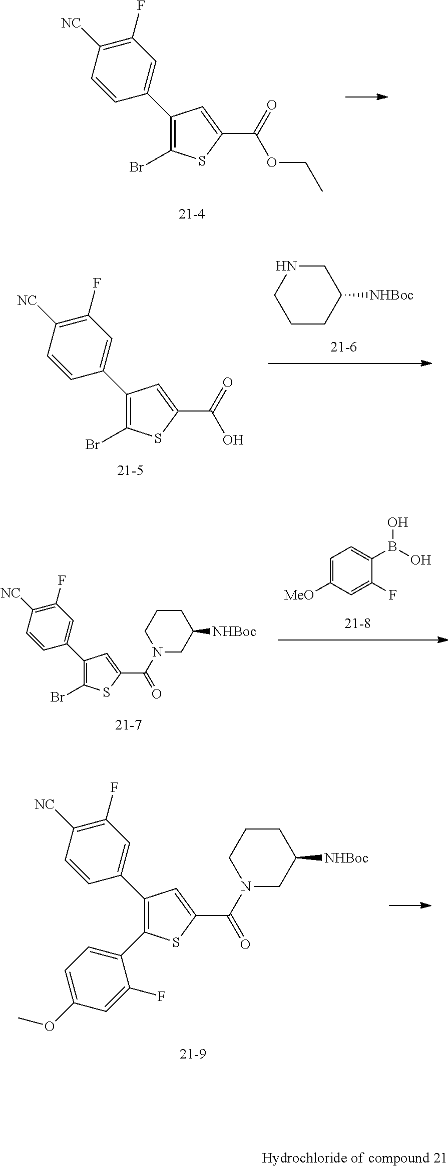

### Step 1

Under nitrogen atmosphere, compound 21-1 (15.0 g, 63.8 mmol), compound 21-2 (12.6 g, 76.6 mmol), and sodium carbonate (13.5 g, 128 mmol) were dissolved in dioxane (120 mL) and water (30 mL), and the reaction mixture was added with [1,1′-bis(diphenylphosphino)ferrocene]dichloropalladium(II) (2.33 g, 3.19 mmol), stirred, and reacted for 12 hours at 100° C. The reaction mixture was concentrated under reduced pressure, and the residue was purified by silica gel column chromatography (4/1, petroleum ether/ethyl acetate, R_{f}=0.53) to obtain compound 21-3. ¹H NMR (400 MHz, CD₃OD) δ=8.23 (dd, J=1.6, 6.4 Hz, 2H), 7.83-7.69 (m, 3H), 4.38 (q, J=7.2 Hz, 2H), 1.39 (t, J=7.2 Hz, 3H).

### Step 2

Compound 21-3 (10.0 g, 3.21 mmol) was dissolved in acetic acid (120 mL) and sulfuric acid (50.0 mL). N-Bromosuccinimide (12.9 g, 72.6 mmol) was added thereto, and the reaction mixture was stirred and reacted for 12 hours at 25° C. The reaction mixture was poured into water, filtered, and the filter cake was dried, and the resulting crude product was purified by silica gel column chromatography (3/1, petroleum ether/ethyl acetate, R_{f}=0.72) to obtain compound 21-4. ¹H NMR (400 MHz, CD₃Cl) δ=7.75-7.68 (m, 2H), 7.49-7.43 (m, 2H), 4.38 (q, J=7.2 Hz, 2H), 1.39 (t, J=7.2 Hz, 3H).

### Step 3

Compound 21-4 (10.8 g, 30.5 mmol) was dissolved in tetrahydrofuran (100 mL) and water (100 mL), and lithium hydroxide monohydrate (3.84 g, 91.5 mmol) was added thereto, and the reaction mixture was stirred and reacted for 14 hours at 25° C. After the reaction mixture was concentrated, the residue was added with water (200 mL). The mixture was acidified to pH=3 to 4 with 1 M dilute hydrochloric acid, and extracted with ethyl acetate (200 mL×3). The organic phases were combined, dried over anhydrous sodium sulfate, filtered, and concentrated under reduced pressure to obtain crude compound 21-5, which was used directly in the next reaction step.

### Step 4

Compound 21-5 (6.00 g, 15.3 mmol) and compound 21-6 (3.67 g, 18.3 mmol) were dissolved in N,N-dimethylformamide (150 mL), and then 2-(7-azabenzotriazol-1-yl)-N,N,N′,N′-tetramethyluronium hexafluorophosphate (8.71 g, 22.9 mmol) and N,N-diisopropylethylamine (209 mg, 1.62 mmol) were added thereto, and the reaction mixture was stirred and reacted for 12 hours at 25° C. The reaction mixture was added with water (200 mL), and extracted with ethyl acetate (200 mL×2). The organic phases were combined, washed with saturated brine (200 mL×1), dried over anhydrous sodium sulfate, filtered, and concentrated. The resulting crude product was purified by column chromatography (1/2, petroleum ether/ethyl acetate, R_{f}=0.25) to obtain compound 21-7. ¹H NMR (400 MHz, CD₃Cl) δ=7.85-7.53 (m, 4H), 4.72-4.22 (m, 2H), 3.71-3.60 (m, 1H), 3.07-2.94 (m, 2H), 2.15-1.78 (m, 2H), 1.73-1.49 (m, 2H), 1.43 (s, 9H). MS-ESI calculated for [M+H]⁺ 510, found 510.

### Step 5

Under nitrogen atmosphere, compound 21-7 (50.0 mg, 98.4 μmol), compound 21-8 (20.0 mg, 118 μmol), and sodium carbonate (20.8 mg, 197 μmol) were dissolved in dioxane (8.00 mL) and water (2.00 mL), and the reaction mixture was added with [1,1′-bis(diphenylphosphino)ferrocene]dichloropalladium(II) (7.20 mg, 9.83 μmol), and the reaction mixture was stirred and reacted for 12 hours at 100° C. The reaction mixture was concentrated under reduced pressure, and the residue was purified by silica gel column chromatography (1/1, petroleum ether/ethyl acetate, R_{f}=0.57) to obtain compound 21-9. ¹H NMR (400 MHz, CD₃OD) δ=7.89-7.69 (m, 1H), 7.64-7.44 (m, 2H), 7.32-7.24 (m, 2H), 6.83 (dd, J=2.4, 8.8 Hz, 1H), 6.74 (dd, J=2.4, 12.0 Hz, 1H), 4.49-4.37 (m, 1H), 4.33-4.36 (m, 1H), 3.83 (s, 3H), 3.71-3.55 (m, 1H), 3.12-2.99 (m, 2H), 2.04-1.96 (m, 1H), 1.93-1.88 (m, 1H), 1.69-1.53 (m, 2H), 1.54 (s, 9H). MS-ESI calculated for [M+Na]⁺ 576, found 576.

### Step 6

Compound 21-9 (50.0 mg, 90.3 μmol) was dissolved in ethyl acetate (15.0 mL), and hydrochloride ethyl acetate solution (4 mol/L, 226 μL) was added thereto, and the reaction mixture was stirred and reacted for 12 hours at 25° C. After the reaction mixture was concentrated, the residue was purified by preparative high performance liquid chromatography (chromatographic column: Phenomenex Synergi C18 150*30 mm*4 μm; mobile phase A: hydrochloric acid aqueous solution with a volume fraction of 0.05%; mobile phase B: acetonitrile; B %: 36% to 66%, 9 minutes) to obtain the hydrochloride of compound 21. ¹H NMR (400 MHz, CD₃OD) δ=7.73-7.65 (m, 1H), 7.64 (s, 1H), 7.38-7.28 (m, 2H), 7.25 (dd, J=1.6, 8.0 Hz, 1H), 6.87 (dd, J=2.4, 8.4 Hz, 1H), 6.78 (dd, J=2.4, 12.0 Hz, 1H), 4.39 (d, J=11.2 Hz, 1H), 4.19-4.13 (m, 1H), 3.86 (s, 3H), 3.61-3.39 (m, 3H), 2.23-2.19 (m, 1H), 1.97-1.86 (m, 1H), 1.85-1.69 (m, 2H). MS-ESI calculated for [M+H]⁺ 454, found 454.

### Example 22

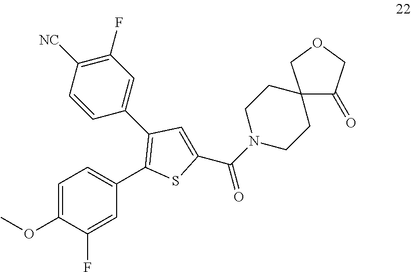 [SMILES: COc1ccc(-c2sc(C(=O)N3CCC4(CC3)COCC4=O)cc2-c2ccc(C#N)c(F)c2)cc1F]

Synthetic Route:

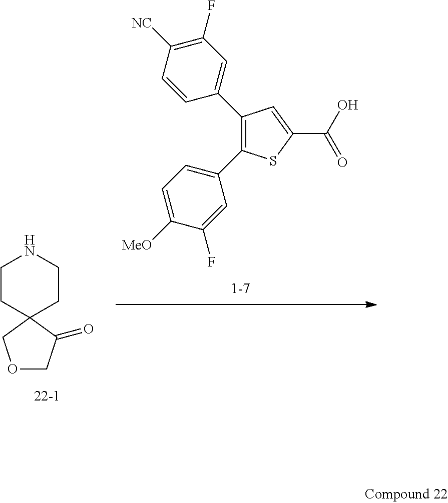 [SMILES: O=C1COCC12CCNCC2 ; COc1ccc(-c2sc(C(=O)O)cc2-c2ccc(C#N)c(F)c2)cc1F]

Compound 1-7 (190 mg, 0.512 mmol) was dissolved in anhydrous dichloromethane (5 mL), and then triethylamine (152.87 mg, 1.18 mmol), compound 22-1 (98.1 mg, 0.512 mmol), and 2-(7-azabenzotriazol-1-yl)-N,N,N,N′-tetramethyluronium hexafluorophosphate (292 mg, 0.767 mmol) were added thereto, and the reaction mixture was stirred for 12 hours at 25° C. The reaction mixture was added with water (20 mL) and extracted with dichloromethane (20 mL×1). The organic phase was washed with saturated brine (20 mL×1), dried over anhydrous sodium sulfate, filtered, and concentrated under reduced pressure. The residue was separated by preparative high performance liquid chromatography (chromatographic column: Phenomenex Synergi C18 150*30 mm*4 μm; mobile phase: mobile phase A: hydrochloric acid aqueous solution with a volume fraction of 0.05%; mobile phase B: acetonitrile; B %: 54% to 84%, 9 minutes) to obtain compound 22. ¹H NMR (400 MHz, CD₃OD) δ 7.68 (t, J=7.6 Hz, 1H), 7.51 (s, 1H), 7.34-7.29 (m, 1H), 7.27-7.22 (m, 1H), 7.14-7.02 (m, 3H), 4.20-4.10 (m, 4H), 4.05 (s, 2H), 3.89 (s, 3H), 3.70-3.50 (m, 2H), 1.88-1.78 (m, 2H), 1.77-1.67 (m, 2H). MS-ESI calculated for [M+H]⁺ 509, found 509.

### Example 23

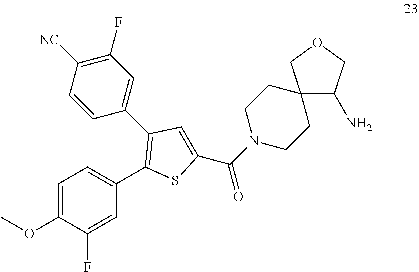 [SMILES: COc1ccc(-c2sc(C(=O)N3CCC4(CC3)COCC4N)cc2-c2ccc(C#N)c(F)c2)cc1F]

Synthetic Route:

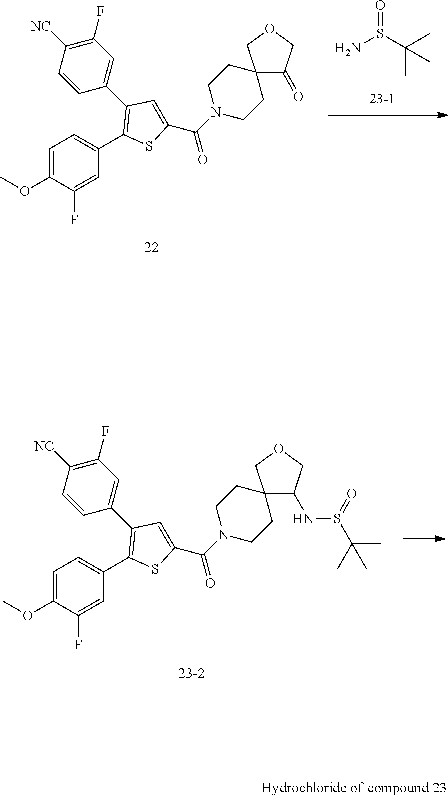

### Step 1

Under nitrogen atmosphere, compound 22 (130 mg, 0.256 mmol) was dissolved in anhydrous tetrahydrofuran (10 mL), and then titanium ethoxide (233 mg, 1.02 mmol) and tert-butanesulfinamide (23-1) (62.0 mg, 0.511 mmol) were added thereto, and the reaction mixture was stirred for 12 hours at 85° C., then cooled to −5° C., and methanol (73.7 mg, 2.30 mmol) was added thereto, and then lithium borohydride (62.0 mg, 0.511 mmol) was slowly added thereto. The reaction mixture was stirred for 1 hour at −5° C., cooled to 0° C., added dropwise with saturated ammonium chloride aqueous solution (20 mL), then added with ethyl acetate (20 mL), and filtrated. The filter cake was washed with ethyl acetate (10 mL). The organic phase was washed with saturated brine (20 mL×1), dried over anhydrous sodium sulfate, filtered, concentrated under reduced pressure, and the residue was separated by silica gel column chromatography (10/1, dichloromethane/methanol, R_{f}=0.43) to obtain compound 23-2. MS-ESI calculated for [M+H]⁺ 614, found 614.

### Step 2

Compound 23-2 (60.0 mg, 0.098 mmol) was dissolved in ethyl acetate (5 mL), and hydrochloride ethyl acetate solution (4 mol/L, 5 mL) was added dropwise thereto at 25° C. The reaction mixture was stirred for 12 hours at 25° C. and concentrated under reduced pressure. The residue was separated by preparative high performance liquid chromatography (chromatographic column: Phenomenex Synergi C18 150*30 mm*4 μm; mobile phase: mobile phase A: hydrochloric acid aqueous solution with a volume fraction of 0.05%; mobile phase B: acetonitrile; B %: 32% to 62%, 9 minutes) to obtain the hydrochloride of compound 23. ¹H NMR (400 MHz, CD₃OD) δ 7.69 (t, J=7.6 Hz, 1H), 7.54-7.50 (m, 1H), 7.35-7.30 (m, 1H), 7.27-7.23 (m, 1H), 7.15-7.09 (m, 1H), 7.08-7.03 (m, 2H), 4.41-4.24 (m, 2H), 4.22-4.16 (m, 1H), 4.01-3.96 (m, 1H), 3.94-3.91 (m, 1H), 3.90 (s, 3H), 3.86-3.82 (m, 1H), 3.64-3.60 (m, 1H), 3.52-3.37 (m, 1H), 1.96-1.73 (m, 4H). MS-ESI calculated for [M+H]⁺ 510, found 510.

### Example 24

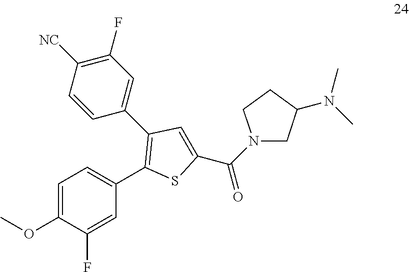

Synthetic Route:

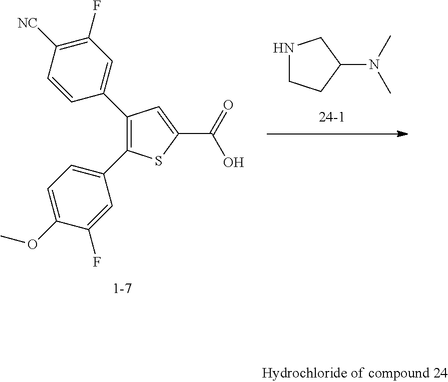

Under nitrogen atmosphere, compound 1-7 (100 mg, 269 μmol), 2-(7-azabenzotriazol-1-yl)-N,N,N′,N′-tetramethyluronium hexafluorophosphate (154 mg, 404 μmol), and N,N-diisopropylethylamine (104 mg, 807 μmol) were dissolved in N,N-dimethylformamide (10.00 mL), and then compound 24-1 (36.9 mg, 323 μmol) was added thereto, and the reaction mixture was stirred and reacted for 12 hours at 25° C. The reaction mixture was added with water (50.0 mL), and extracted with ethyl acetate (50.0 mL×2). The organic phase was washed with saturated brine (50.0 mL×1), dried over anhydrous sodium sulfate, filtered, and concentrated under reduced pressure. The residue was purified by preparative high performance liquid chromatography (chromatographic column: Xtimate C18 150*40 mm*5 μm; mobile phase A: hydrochloric acid aqueous solution with a volume fraction of 0.05%; mobile phase B: acetonitrile; B %: 14% to 44%, 10 minutes) to obtain the hydrochloride of compound 24. ¹H NMR (400 MHz, CD₃OD) δ=7.74 (s, 1H), 7.72-7.67 (m, 1H), 7.34 (d, J=10.0 Hz, 1H), 7.26 (d, J=7.6 Hz, 1H), 7.16-7.01 (m, 3H), 4.53-4.13 (m, 2H), 4.10-3.95 (m, 2H), 3.90 (s, 3H), 3.87-3.79 (m, 1H), 3.00 (s, 6H), 2.63-2.59 (m, 1H), 2.36-2.31 (m, 1H). MS-ESI calculated for [M+H]⁺ 468, found 468.

### Example 25

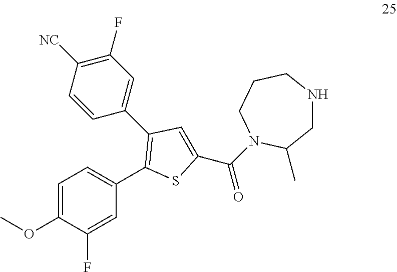 [SMILES: COc1ccc(-c2sc(C(=O)N3CCCNCC3C)cc2-c2ccc(C#N)c(F)c2)cc1F]

Synthetic Route:

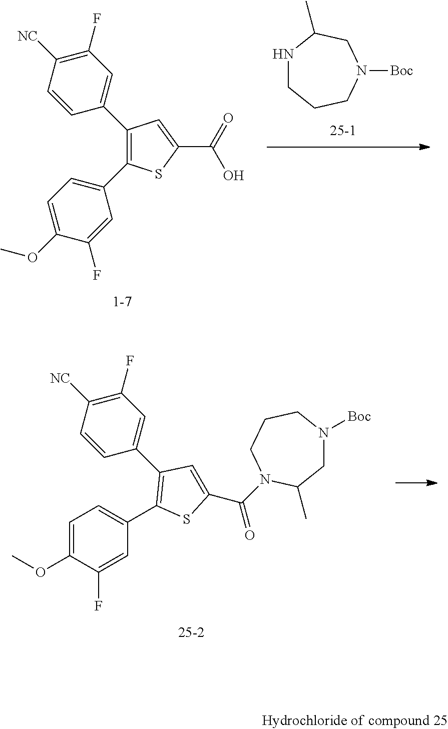 [SMILES: COc1ccc(-c2sc(C(=O)O)cc2-c2ccc(C#N)c(F)c2)cc1F ; CC1CN(C(=O)OC(C)(C)C)CCCN1 ; COc1ccc(-c2sc(C(=O)N3CCCN(C(=O)OC(C)(C)C)CC3C)cc2-c2ccc(C#N)c(F)c2)cc1F]

### Step 1

Compound 1-7 (100 mg, 269 μmol), 2-(7-azabenzotriazol-1-yl)-N,N,N′,N′-tetramethyluronium hexafluorophosphate (154 mg, 404 μmol), and N,N-diisopropylethylamine (104 mg, 807 μmol) were dissolved in N,N-dimethylformamide (20.0 mL), then compound 25-1 (69.3 mg, 323 μmol) was added thereto, and the reaction mixture was stirred and reacted for 6 hours at 25° C. The reaction mixture was added with water (30.0 mL), and extracted with ethyl acetate (30.0 mL×2). The organic phase was washed with saturated brine (30.0 mL×1), dried over anhydrous sodium sulfate, filtered, and concentrated under reduced pressure. The residue was purified by silica gel column chromatography (1/1, petroleum ether/ethyl acetate, R_{f}=0.50) to obtain compound 25-2. MS-ESI calculated for [M−56+H]⁺ 512, found 512.

### Step 2

Compound 25-2 (147 mg, 259 μmol) was dissolved in ethyl acetate (30.0 mL), and hydrochloride ethyl acetate solution (4 mol/L, 324 μL) was added thereto, and the reaction mixture was stirred and reacted for 6 hours at 25° C. After the reaction mixture was concentrated, the residue was purified by preparative high performance liquid chromatography (chromatographic column: Xtimate C18 150*40 mm*5 μm; mobile phase A: hydrochloric acid aqueous solution with a volume fraction of 0.05%; mobile phase B: acetonitrile; B %: 17% to 47%, 10 minutes) to obtain the hydrochloride of compound 25. ¹H NMR (400 MHz, CD₃OD) δ=7.76-7.69 (m, 1H), 7.59 (s, 1H), 7.35 (dd, J=1.2, 10.2 Hz, 1H), 7.28 (dd, J=1.6, 8.0 Hz, 1H), 7.17-7.05 (m, 3H), 5.03-4.92 (m, 1H), 4.86-4.74 (m, 1H), 4.3-4.22 (m, 1H), 3.92 (s, 3H), 3.79-3.70 (m, 1H), 3.58-3.48 (m, 1H), 3.29-3.22 (m, 1H), 3.21-3.08 (m, 1H), 2.28-2.05 (m, 2H), 1.42-1.28 (s, 3H). MS-ESI calculated for [M+H]⁺ 468, found 468.

### Example 26

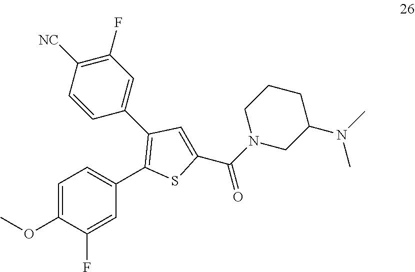

Synthetic Route:

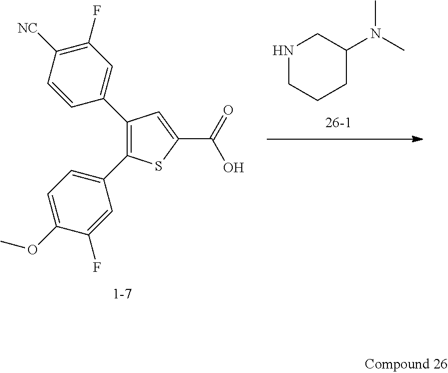

Under nitrogen atmosphere, compound 1-7 (100 mg, 269 μmol), 2-(7-azabenzotriazol-1-yl)-N,N,N′,N′-tetramethyluronium hexafluorophosphate (154 mg, 404 μmol), and N,N-diisopropylethylamine (104 mg, 807 μmol) were dissolved in N,N-dimethylformamide (30.0 mL), and then compound 26-1 (41.4 mg, 323 μmol) was added thereto, and the reaction mixture was stirred and reacted for 12 hours at 25° C. The reaction mixture was added with water (50 mL), and extracted with ethyl acetate (50.0 mL×2). The organic phase was washed with saturated brine (50.0 mL×1), dried over anhydrous sodium sulfate, filtered, and concentrated under reduced pressure. The residue was purified by preparative high performance liquid chromatography (chromatographic column: Xtimate C18 150*40 mm*5 μm; mobile phase A: hydrochloric acid aqueous solution with a volume fraction of 0.05%; mobile phase B: acetonitrile; B %: 16% to 46%, 10 minutes) to obtain the hydrochloride of compound 26. ¹H NMR (400 MHz, CD₃OD) δ=7.69 (t, J=7.6 Hz, 1H), 7.59 (s, 1H), 7.34 (d, J=10.4 Hz, 1H), 7.27 (d, J=8.0 Hz, 1H), 7.15-7.01 (m, 3H), 4.55 (d, J=11.6 Hz, 1H), 4.25 (d, J=11.2 Hz, 1H), 3.89 (s, 3H), 3.65-3.55 (m, 1H), 3.54-3.38 (m, 2H), 3.00 (s, 3H), 2.95 (s, 3H), 2.29-2.25 (m, 1H), 2.03-1.90 (m, 2H), 1.78-1.68 (m, 1H). MS-ESI calculated for [M+H]⁺ 482, found 482.

### Example 27

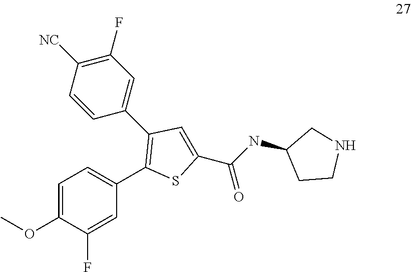 [SMILES: COc1ccc(-c2sc(C(=O)N[C@@H]3CCNC3)cc2-c2ccc(C#N)c(F)c2)cc1F]

Synthetic Route:

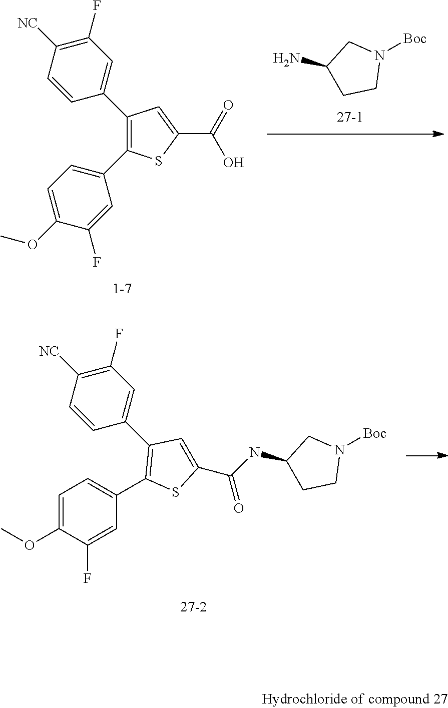 [SMILES: COc1ccc(-c2sc(C(=O)O)cc2-c2ccc(C#N)c(F)c2)cc1F ; CC(C)(C)OC(=O)N1CC[C@@H](N)C1 ; COc1ccc(-c2sc(C(=O)N[C@@H]3CCN(C(=O)OC(C)(C)C)C3)cc2-c2ccc(C#N)c(F)c2)cc1F]

### Step 1

Compound 1-7 (100 mg, 269 μmol), 2-(7-azabenzotriazol-1-yl)-N,N,N′,N′-tetramethyluronium hexafluorophosphate (154 mg, 404 μmol), and N,N-diisopropylethylamine (104 mg, 807 μmol) were dissolved in N,N-dimethylformamide (10.0 mL), then compound 27-1 (60.2 mg, 323 μmol) was added thereto, and the reaction mixture was stirred and reacted at 25° C. for 12 hours. The reaction mixture was added with water (50 mL), and extracted with ethyl acetate (50 mL×2). The organic phase was washed with saturated brine (50 mL×1), dried over anhydrous sodium sulfate, filtered, and concentrated under reduced pressure. The residue was purified by silica gel column chromatography (1/1, petroleum ether/ethyl acetate, R_{f}=0.43) to obtain compound 27-2. MS-ESI calculated for [M−56+H]⁺ 484, found 484.

### Step 2

Compound 27-2 (120 mg, 259 μmol) was dissolved in ethyl acetate (30.0 mL), and hydrochloride ethyl acetate solution (4 mol/L, 278 μL) was added thereto, and the reaction mixture was stirred and reacted for 12 hours at 25° C. After the reaction mixture was concentrated, the residue was purified by preparative high performance liquid chromatography (chromatographic column: Xtimate C18 150*40 mm*5 μm; mobile phase A: hydrochloric acid aqueous solution with a volume fraction of 0.05%; mobile phase B: acetonitrile; B %: 17% to 47%, 10 minutes) to obtain the hydrochloride of compound 27. ¹H NMR (400 MHz, CD₃OD) δ=8.01 (s, 1H), 7.71 (t, J=7.6 Hz, 1H), 7.36-7.21 (m, 2H), 7.18-6.99 (m, 3H), 4.72-4.62 (m, 1H), 3.91 (s, 3H), 3.68-3.55 (m, 2H), 3.52-3.37 (m, 2H), 2.48-2.39 (m, 1H), 2.30-2.22 (m, 1H). MS-ESI calculated for [M+H]⁺ 440, found 440.

### Example 28

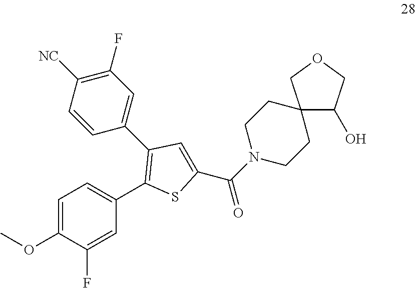 [SMILES: COc1ccc(-c2sc(C(=O)N3CCC4(CC3)COCC4O)cc2-c2ccc(C#N)c(F)c2)cc1F]

Synthetic Route:

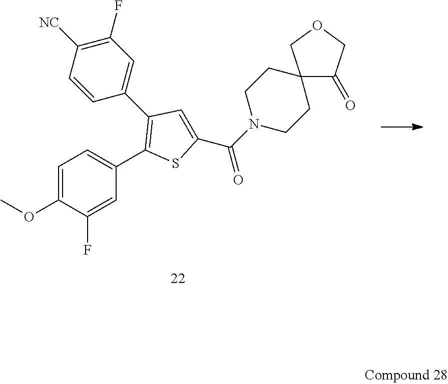 [SMILES: COc1ccc(-c2sc(C(=O)N3CCC4(CC3)COCC4=O)cc2-c2ccc(C#N)c(F)c2)cc1F]

Compound 22 (80.0 mg, 0.157 mmol) was dissolved in anhydrous tetrahydrofuran (5.00 mL), and sodium borohydride (11.9 mg, 0.315 mmol) was added thereto. The reaction mixture was stirred for 12 hours at 25° C., cooled to 0° C., added dropwise with saturated ammonium chloride aqueous solution (20 mL), and then added with ethyl acetate (20 mL). The organic phase was washed with saturated brine (20 mL×1), dried over anhydrous sodium sulfate, filtered, concentrated under reduced pressure, and the residue was separated by preparative high performance liquid chromatography (chromatographic column: Phenomenex Synergi C18 150*30 mm*4 μm; mobile phase: mobile phase A: hydrochloric acid aqueous solution with a volume fraction of 0.05%; mobile phase B: acetonitrile; B %: 49% to 79%, 9 minutes) to obtain compound 28. ¹H NMR (400 MHz, CD₃OD) δ 7.72-7.66 (m, 1H), 7.50 (s, 1H), 7.32 (dd, J=1.2, 10.4 Hz, 1H), 7.25 (dd, J=1.6, 8.0 Hz, 1H), 7.15-7.02 (m, 3H), 4.16-4.10 (m, 1H), 4.09-4.05 (m, 1H), 3.99-3.91 (m, 2H), 3.90 (s, 3H), 3.80-3.77 (m, 1H), 3.76-3.57 (m, 4H), 1.97-1.88 (m, 1H), 1.70-1.60 (m, 3H). MS-ESI calculated for [M+H]⁺ 511, found 511.

### Example 29

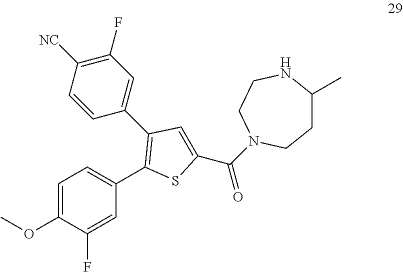

Synthetic Route:

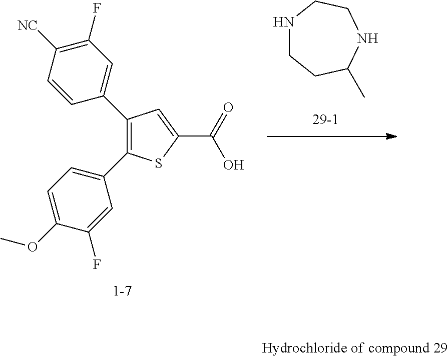

Compound 1-7 (100 mg, 269 μmol), 2-(7-azabenzotriazol-1-yl)-N,N,N′,N′-tetramethyluronium hexafluorophosphate (154 mg, 404 μmol), and N,N-diisopropylethylamine (104 mg, 807 μmol) were dissolved in N,N-dimethylformamide (10.0 mL), then compound 29-1 (61.5 mg, 539 μmol) was added thereto, and the reaction mixture was stirred and reacted at 25° C. for 12 hours. The reaction mixture was added with water (50 mL), and extracted with ethyl acetate (50 mL×2). The organic phase was washed with saturated brine (50 mL×1), dried over anhydrous sodium sulfate, filtered, and concentrated under reduced pressure. The residue was purified by preparative high performance liquid chromatography (chromatographic column: Phenomenex Gemini-NX 80*30 mm*3 μm; mobile phase A: 10 mM NH₄HCO₃ aqueous solution; mobile phase B: acetonitrile; B %: 40% to 70%, 9 minutes), and 200 μL of 2N hydrochloric acid aqueous solution was added thereto to obtain the hydrochloride of compound 29. ¹H NMR (400 MHz, CD₃OD) δ=7.71-7.67 (m, 1H), 7.61 (s, 1H), 7.33 (dd, J=1.2, 10.4 Hz, 1H), 7.25 (dd, J=1.2, 8.4 Hz, 1H), 7.15-7.01 (m, 3H), 4.17-4.10 (m, 2H), 3.99-3.92 (m, 1H), 3.90 (s, 3H), 3.83-3.60 (m, 1H), 3.66-3.50 (m, 2H), 3.4-3.34 (m, 1H), 2.26-2.00 (m, 2H), 1.42 (d, J=6.4 Hz, 3H). MS-ESI calculated for [M+H]⁺ 468, found 468.

### Example 30

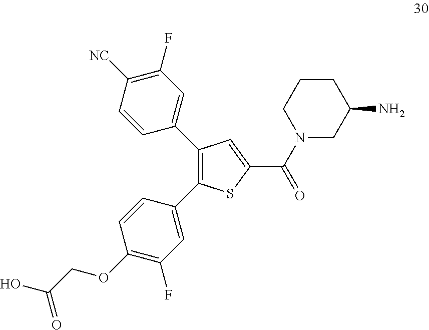 [SMILES: N#Cc1ccc(-c2cc(C(=O)N3CCC[C@@H](N)C3)sc2-c2ccc(OCC(=O)O)c(F)c2)cc1F]

Synthetic Route:

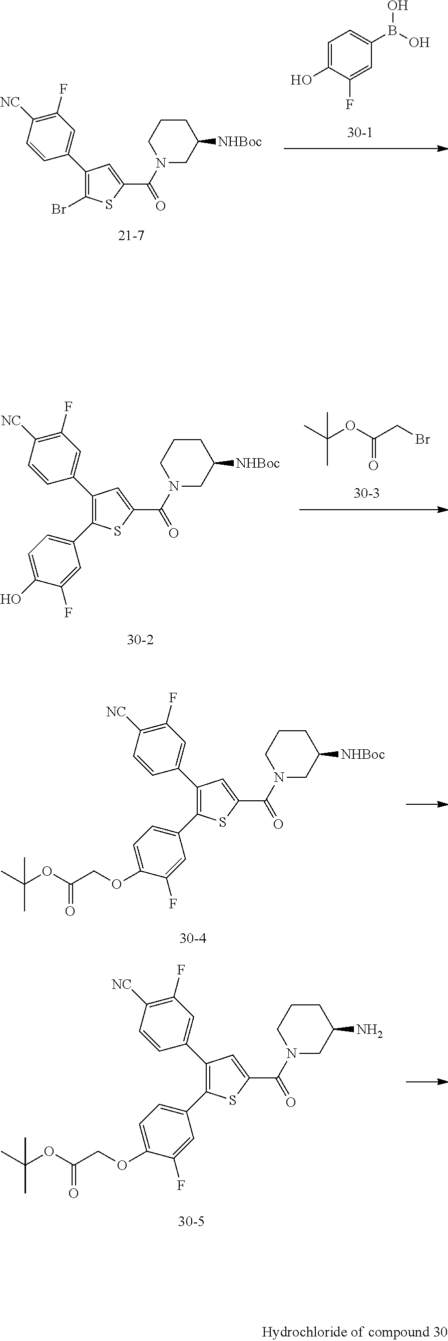

### Step 1

Under nitrogen atmosphere, compound 21-7 (130 mg, 256 μmol), compound 30-1 (47.8 mg, 309 μmol), and sodium carbonate (54.2 mg, 511 μmol) were dissolved in dioxane (8.00 mL) and water (2.00 mL), and the reaction mixture was added with [1,1′-bis(diphenylphosphino)ferrocene]dichloropalladium(II) (18.7 mg, 25.6 μmol), and the reaction mixture was stirred and reacted for 12 hours at 100° C. The reaction mixture was concentrated under reduced pressure, and the residue was purified by silica gel column chromatography (1/2, petroleum ether/ethyl acetate, R_{f}=0.43) to obtain compound 30-2. ¹H NMR (400 MHz, CD₃OD) δ=7.85-7.45 (m, 3H), 7.28 (d, J=8.0 Hz, 1H), 7.04-6.97 (m, 1H), 6.96-6.88 (m, 2H), 4.50-4.39 (m, 1H), 4.32-4.16 (m, 1H), 3.67-3.56 (m, 1H), 3.21-3.01 (m, 2H), 2.07-1.97 (m, 1H), 1.93-1.82 (m, 1H), 1.71-1.52 (m, 2H), 1.42 (s, 9H). MS-ESI calculated for [M−56+H]⁺ 484, found 484.

### Step 2

Compound 30-2 (66.0 mg, 122 μmol) was dissolved in N,N-dimethylformamide (5.00 mL), and the reaction mixture was added with 30-3 (28.6 mg, 147 μmol) and potassium carbonate (33.8 mg, 245 μmol), stirred, and reacted at 60° C. for 12 hours. The reaction mixture was added with water (50 mL), and extracted with ethyl acetate (50 mL×2). The organic phase was washed with saturated brine (50 mL×1), dried over anhydrous sodium sulfate, filtered, and concentrated under reduced pressure. The residue was purified by silica gel column chromatography (1/2, petroleum ether/ethyl acetate, R_{f}=0.42) to obtain compound 30-4. ¹H NMR (400 MHz, CD₃OD) δ=7.88-7.43 (m, 3H), 7.28 (d, J=8.0 Hz, 1H), 7.11-7.05 (m, 1H), 7.06-6.96 (m, 2H), 4.70 (s, 2H), 4.44-4.39 (m, 1H), 4.23-4.20 (m, 1H), 3.63-3.60 (m, 1H), 3.23-2.97 (m, 2H), 2.03-2.00 (m, 1H), 1.94-1.82 (m, 1H), 1.71-1.54 (m, 2H), 1.48 (s, 9H), 1.42 (s, 9H). MS-ESI calculated for [M+H]⁺ 654, found 654.

### Step 3

Compound 30-4 (50.0 mg, 259 μmol) was dissolved in ethyl acetate (5.00 mL), and hydrochloride ethyl acetate solution (4 mol/L, 191 μL) was added thereto, and the reaction mixture was stirred and reacted for 12 hours at 25° C. The reaction mixture was concentrated to obtain crude compound 30-5, which was used directly in the next reaction step. MS-ESI calculated for [M+H]⁺ 554, found 554.

### Step 4

Compound 30-5 (50.0 mg, 84.7 μmol) was dissolved in dichloromethane (5.00 mL), and trifluoroacetic acid (2.00 mL) was added thereto, and the reaction mixture was stirred and reacted for 2 hours at 25° C. After the reaction mixture was concentrated, the residue was separated by preparative high performance liquid chromatography (chromatographic column: Phenomenex Synergi C18 150*30 mm*4 μm; mobile phase A: hydrochloric acid aqueous solution with a volume fraction of 0.05%; mobile phase B: acetonitrile; B %: 17% to 47%, 10 minutes) to obtain the hydrochloride of compound 30. ¹H NMR (400 MHz, CD₃OD) δ=7.70 (t, J=7.6 Hz, 1H), 7.56 (s, 1H), 7.34 (d, J=10.4 Hz, 1H), 7.25 (dd, J=1.2, 8.2 Hz, 1H), 7.13-6.97 (m, 3H), 4.78 (s, 2H), 4.37 (d, J=11.6 Hz, 1H), 4.14 (d, J=13.6 Hz, 1H), 3.61-3.35 (m, 3H), 2.26-2.14 (m, 1H), 1.95-1.84 (m, 1H), 1.83-1.67 (m, 2H). MS-ESI calculated for [M+H]⁺ 498, found 498.

### Example 31

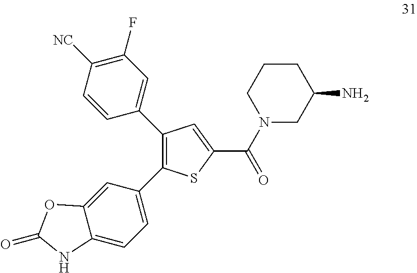 [SMILES: N#Cc1ccc(-c2cc(C(=O)N3CCC[C@@H](N)C3)sc2-c2ccc3[nH]c(=O)oc3c2)cc1F]

Synthetic Route:

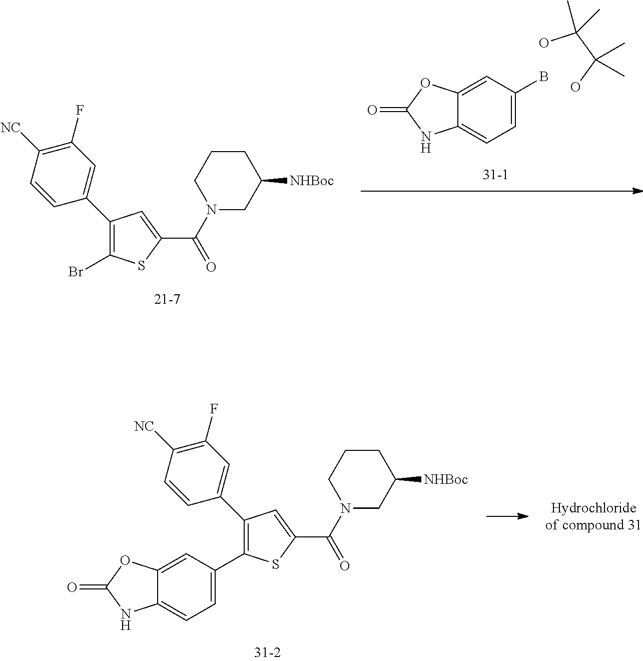

### Step 1

Under nitrogen atmosphere, compound 21-7 (81.1 mg, 160 μmol), compound 31-1 (50.0 mg, 192 μmol), and sodium carbonate (33.8 mg, 319 μmol) were dissolved in dioxane (4.00 mL) and water (1.00 mL), and the reaction mixture was added with [1,1′-bis(diphenylphosphino)ferrocene]dichloropalladium(II) (11.7 mg, 16.0 μmol), and the reaction mixture was stirred and reacted for 12 hours at 100° C. The reaction mixture was concentrated under reduced pressure, and the residue was purified by silica gel column chromatography (1/2, petroleum ether/ethyl acetate, R_{f}=0.14) to obtain compound 31-2. MS-ESI calculated for [M−56+H]⁺ 507, found 507.

### Step 2

Compound 31-2 (50 mg, 88.8 μmol) was dissolved in ethyl acetate (20.0 mL), and hydrochloride ethyl acetate solution (4 mol/L, 222 μL) was added thereto, and the reaction mixture was stirred and reacted for 12 hours at 25° C. After the reaction mixture was concentrated, the residue was purified by preparative high performance liquid chromatography (chromatographic column: Xtimate C18 150*40 mm*5 μm; mobile phase A: hydrochloric acid aqueous solution with a volume fraction of 0.05%; mobile phase B: acetonitrile; B %: 8% to 38%, 10 minutes) to obtain the hydrochloride of compound 31. ¹H NMR (400 MHz, CD₃OD) δ=7.72-7.65 (m, 1H), 7.60 (s, 1H), 7.39-7.33 (m, 1H), 7.28-7.24 (m, 1H), 7.21 (s, 1H), 7.16-7.05 (m, 2H), 4.42-4.35 (m, 1H), 4.20-4.13 (m, 1H), 3.54-3.38 (m, 3H), 2.30-2.19 (m, 1H), 1.97-1.89 (m, 1H), 1.85-1.68 (m, 2H). MS-ESI calculated for [M+H]⁺ 463, found 463.

### Example 32

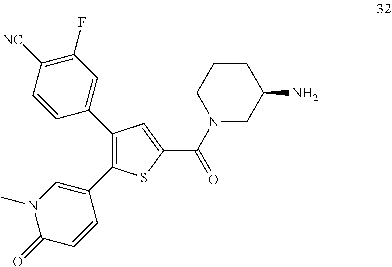 [SMILES: Cn1cc(-c2sc(C(=O)N3CCC[C@@H](N)C3)cc2-c2ccc(C#N)c(F)c2)ccc1=O]

Synthetic Route:

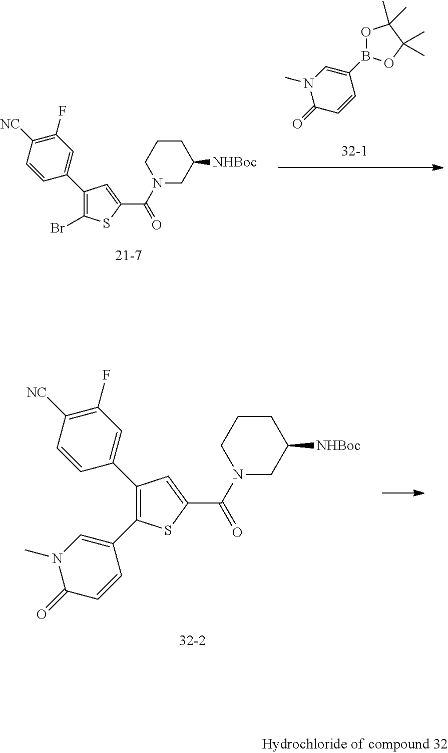

### Step 1

Under nitrogen atmosphere, compound 21-7 (180 mg, 354 μmol), compound 32-1 (100 mg, 192 μmol), and sodium carbonate (75.1 mg, 709 μmol) were dissolved in dioxane (8.00 mL) and water (2.00 mL), and the reaction mixture was added with [1,1′-bis(diphenylphosphino)ferrocene]dichloropalladium(II) (25.9 mg, 35.5 μmol), and the reaction mixture was stirred and reacted for 12 hours at 100° C. The reaction mixture was concentrated under reduced pressure, and the residue was purified by silica gel column chromatography (0/1, petroleum ether/ethyl acetate, R_{f}=0.14) to obtain compound 32-2. MS-ESI calculated for [M+H]⁺ 537, found 537.

### Step 2

Compound 32-2 (50.0 mg, 93.18 μmol) was dissolved in ethyl acetate (10.00 mL), and hydrochloride ethyl acetate solution (4 mol/L, 234 μL) was added thereto, and the reaction mixture was stirred and reacted for 12 hours at 25° C. After the reaction mixture was concentrated, the residue was purified by preparative high performance liquid chromatography (chromatographic column: Phenomenex Synergi C18 150*30 mm*4 μm, mobile phase A: hydrochloric acid aqueous solution with a volume fraction of 0.05%; mobile phase B: acetonitrile; B %: 23% to 53%, 9 minutes) to obtain the hydrochloride of compound 32. ¹H NMR (400 MHz, CD₃OD) δ=7.91 (d, J=2.4 Hz, 1H), 7.74 (t, J=7.6 Hz, 1H), 7.59 (s, 1H), 7.45 (d, J=10.4 Hz, 1H), 7.36 (d, J=8.0 Hz, 1H), 7.28 (dd, J=2.4, 9.6 Hz, 1H), 6.51 (d, J=9.6 Hz, 1H), 4.39 (d, J=11.2 Hz, 1H), 4.13 (d, J=13.2 Hz, 1H), 3.59 (s, 3H), 3.53-3.37 (m, 3H), 2.23-2.17 (m, 1H), 1.96-1.85 (m, 1H), 1.82-1.65 (m, 2H). MS-ESI calculated for [M+H]⁺ 437, found 437.

### Example 33

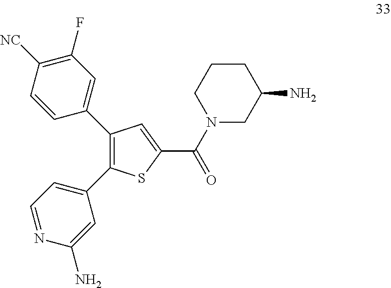 [SMILES: N#Cc1ccc(-c2cc(C(=O)N3CCC[C@@H](N)C3)sc2-c2ccnc(N)c2)cc1F]

Synthetic Route:

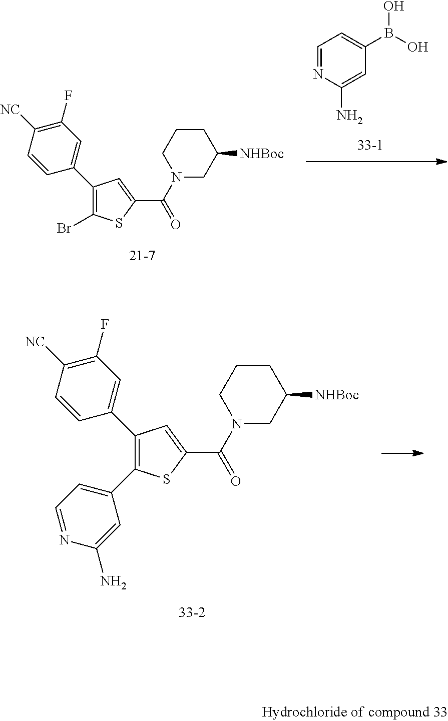 [SMILES: CC(C)(C)OC(=O)N[C@@H]1CCCN(C(=O)c2cc(-c3ccc(C#N)c(F)c3)c(Br)s2)C1 ; Nc1cc(B(O)O)ccn1 ; CC(C)(C)OC(=O)N[C@@H]1CCCN(C(=O)c2cc(-c3ccc(C#N)c(F)c3)c(-c3ccnc(N)c3)s2)C1]

### Step 1

Under nitrogen atmosphere, compound 21-7 (154 mg, 302 μmol), compound 33-1 (50.0 mg, 363 μmol), and sodium carbonate (64.0 mg, 604 μmol) were dissolved in dioxane (4.00 mL) and water (1.00 mL), and the reaction mixture was added with [1,1′-bis(diphenylphosphino)ferrocene]dichloropalladium(II) (22.1 mg, 30.2 μmol), and the reaction mixture was stirred and reacted for 12 hours at 100° C. The reaction mixture was concentrated under reduced pressure, and the residue was purified by silica gel column chromatography (0/1, petroleum ether/ethyl acetate, R_{f}=0.17) to obtain compound 33-2. MS-ESI calculated for [M+H]⁺ 522, found 522.

### Step 2

Compound 33-2 (80.0 mg, 153 μmol) was dissolved in ethyl acetate (5.00 mL), and hydrochloride ethyl acetate solution (4 mol/L, 383 μL) was added thereto, and the reaction mixture was stirred and reacted for 12 hours at 25° C. After the reaction mixture was concentrated, the residue was purified by preparative high performance liquid chromatography (chromatographic column: Xtimate C18 150*40 mm*5 μm, mobile phase A: hydrochloric acid aqueous solution with a volume fraction of 0.05%; mobile phase B: acetonitrile; B %: 1% to 30%, 10 minutes) to obtain the hydrochloride of compound 33. ¹H NMR (400 MHz, CD₃OD) δ=7.87-7.74 (m, 2H), 7.64 (s, 1H), 7.52 (d, J=10.0 Hz, 1H), 7.39 (dd, J=1.2, 8.0 Hz, 1H), 6.96 (d, J=1.2 Hz, 1H), 6.70 (dd, J=1.6, 6.8 Hz, 1H), 4.39 (d, J=11.6 Hz, 1H), 4.25-3.99 (m, 1H), 3.56-3.36 (m, 3H), 2.26-2.12 m, 1H), 1.93-1.80 (m, 1H), 1.79-1.62 (m, 2H). MS-ESI calculated for [M+H]⁺ 422, found 422.

### Example 34

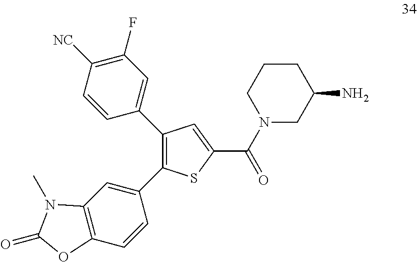 [SMILES: Cn1c(=O)oc2ccc(-c3sc(C(=O)N4CCC[C@@H](N)C4)cc3-c3ccc(C#N)c(F)c3)cc21]

Synthetic Route:

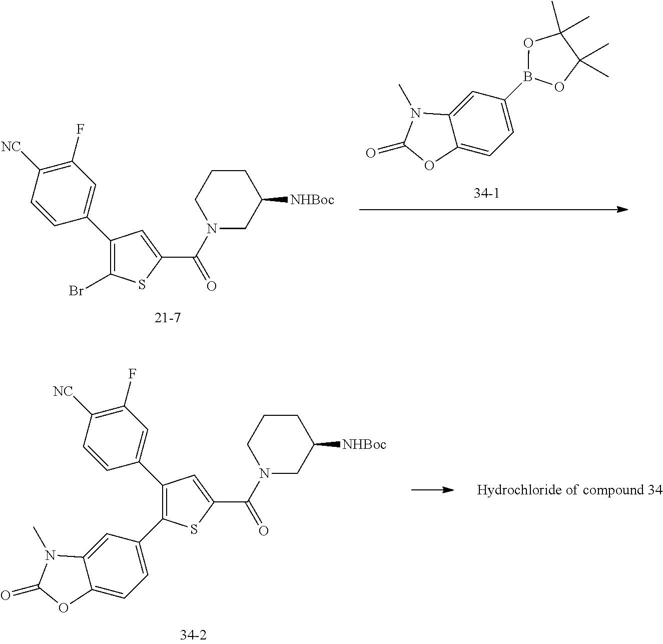 [SMILES: CC(C)(C)OC(=O)N[C@@H]1CCCN(C(=O)c2cc(-c3ccc(C#N)c(F)c3)c(Br)s2)C1 ; Cn1c(=O)oc2ccc(B3OC(C)(C)C(C)(C)O3)cc21 ; Cn1c(=O)oc2ccc(-c3sc(C(=O)N4CCC[C@@H](NC(=O)OC(C)(C)C)C4)cc3-c3ccc(C#N)c(F)c3)cc21]

### Step 1

Under nitrogen atmosphere, compound 21-7 (100 mg, 197 μmol), compound 34-1 (64.9 mg, 236 μmol), and sodium carbonate (41.7 mg, 393 μmol) were dissolved in dioxane (4.00 mL) and water (1.00 mL), and the reaction mixture was added with [1,1′-bis(diphenylphosphino)ferrocene]dichloropalladium(II) (14.4 mg, 19.7 μmol), and the reaction mixture was stirred and reacted for 12 hours at 100° C. The reaction mixture was concentrated under reduced pressure, and the residue was purified by silica gel column chromatography (0/1, petroleum ether/ethyl acetate, R_{f}=0.08) to obtain compound 34-2. MS-ESI calculated for [M−56+H]⁺ 521, found 521.

### Step 2

Compound 34-2 (50.0 mg, 32.1 μmol) was dissolved in ethyl acetate (5.00 mL), and hydrochloride ethyl acetate solution (4 mol/L, 80.0 μL) was added thereto, and the reaction mixture was stirred and reacted for 12 hours at 25° C. After the reaction mixture was concentrated, the residue was purified by preparative high performance liquid chromatography (chromatographic column: Phenomenex Synergi C18 150*30 mm*4 μm, mobile phase A: hydrochloric acid aqueous solution with a volume fraction of 0.05%; mobile phase B: acetonitrile; B %: 35% to 65%, 9 minutes) to obtain the hydrochloride of compound 34. ¹H NMR (400 MHz, CD₃OD) δ=7.71-7.64 (m, 1H), 7.61 (s, 1H), 7.35 (dd, J=1.2, 10.4 Hz, 1H), 7.29-7.22 (m, 2H), 7.17 (d, J=2.0 Hz, 1H), 7.06 (dd, J=2.0, 8.4 Hz, 1H), 4.39 (d, J=11.2 Hz, 1H), 4.16 (d, J=13.6 Hz, 1H), 3.64-3.39 (m, 3H), 3.35 (s, 3H), 2.25-2.14 (m, 1H), 1.97-1.85 (m, 1H), 1.83-1.68 (m, 2H). MS-ESI calculated for [M+H]⁺ 477, found 477.

### Example 35

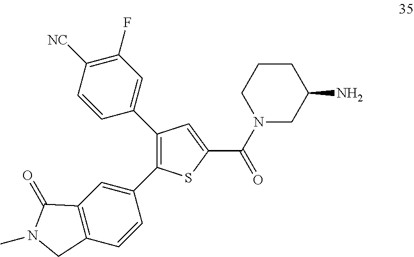 [SMILES: CN1Cc2ccc(-c3sc(C(=O)N4CCC[C@@H](N)C4)cc3-c3ccc(C#N)c(F)c3)cc2C1=O]

Synthetic Route:

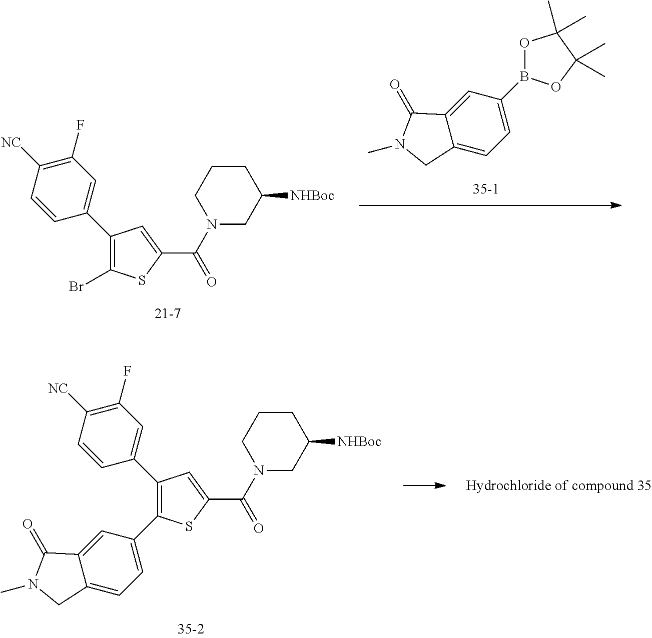

### Step 1

Under nitrogen atmosphere, compound 21-7 (77.6 mg, 153 μmol), compound 35-1 (50.0 mg, 183 μmol), and sodium carbonate (32.3 mg, 305 μmol) were dissolved in dioxane (4.00 mL) and water (1.00 mL), and the reaction mixture was added with [1,1′-bis(diphenylphosphino)ferrocene]dichloropalladium(II) (11.1 mg, 15.2 μmol), and the reaction mixture was stirred and reacted for 12 hours at 100° C. The reaction mixture was concentrated under reduced pressure, and the residue was purified by silica gel column chromatography (1/1, petroleum ether/ethyl acetate, R_{f}=0.54) to obtain compound 35-2. MS-ESI calculated for [M−56+H]⁺ 519, found 519.

### Step 2

Compound 35-2 (65.0 mg, 32.1 μmol) was dissolved in ethyl acetate (5.00 mL), and hydrochloride ethyl acetate solution (4 mol/L, 287 μL) was added thereto, and the reaction mixture was stirred and reacted for 2 hours at 25° C. After the reaction mixture was concentrated, the residue was purified by preparative high performance liquid chromatography (chromatographic column: Xtimate C18 150*40 mm*5 μm, mobile phase A: hydrochloric acid aqueous solution with a volume fraction of 0.05%; mobile phase B: acetonitrile; B %: 10% to 40%, 10 minutes) to obtain the hydrochloride of compound 35. ¹H NMR (400 MHz, CD₃OD) δ=7.73-7.65 (m, 2H), 7.64-7.60 (m, 2H), 7.56 (dd, J=1.6, 8.0 Hz, 1H), 7.34 (dd, J=1.2, 10.4 Hz, 1H), 7.25 (dd, J=1.2, 8.0 Hz, 1H), 4.57 (s, 2H), 4.40 (d, J=12.0 Hz, 1H), 4.17 (d, J=12.4 Hz, 1H), 3.63-3.41 (m, 3H), 3.22 (s, 3H), 2.30-2.14 (m, 1H), 2.01-1.87 (m, 1H), 1.84-1.65 (m, 2H). MS-ESI calculated for [M+H]⁺ 475, found 475.

### Example 36

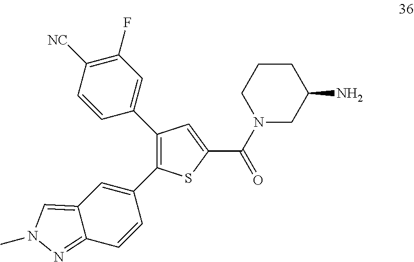 [SMILES: Cn1cc2cc(-c3sc(C(=O)N4CCC[C@@H](N)C4)cc3-c3ccc(C#N)c(F)c3)ccc2n1]

Synthetic Route:

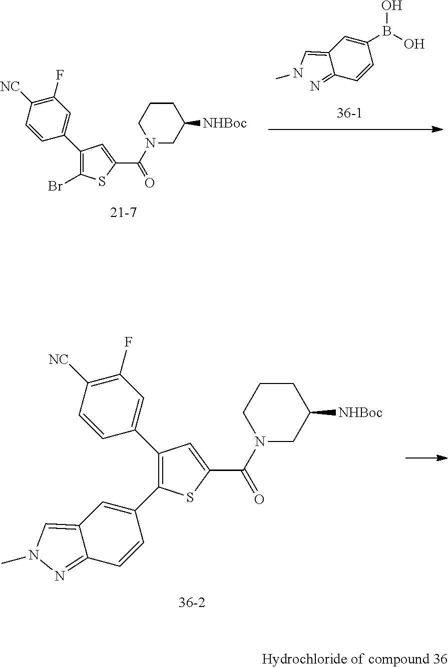 [SMILES: CC(C)(C)OC(=O)N[C@@H]1CCCN(C(=O)c2cc(-c3ccc(C#N)c(F)c3)c(Br)s2)C1 ; CN/C=C1/C=C(B(O)O)C=CC1=N ; Cn1cc2cc(-c3sc(C(=O)N4CCC[C@@H](NC(=O)OC(C)(C)C)C4)cc3-c3ccc(C#N)c(F)c3)ccc2n1]

### Step 1

Under nitrogen atmosphere, compound 21-7 (241 mg, 474 μmol), compound 36-1 (100 mg, 568 μmol), and sodium carbonate (100 mg, 947 μmol) were dissolved in dioxane (8.00 mL) and water (2.00 mL), and the reaction mixture was added with [1,1′-bis(diphenylphosphino)ferrocene]dichloropalladium(II) (34.6 mg, 47.3 μmol), and the reaction mixture was stirred and reacted for 12 hours at 100° C. The reaction mixture was concentrated under reduced pressure, and the residue was purified by silica gel column chromatography (1/1, petroleum ether/ethyl acetate, R_{f}=0.29) to obtain compound 36-2. MS-ESI calculated for [M+H]⁺ 560, found 560.

### Step 2

Compound 36-2 (200 mg, 357 μmol) was dissolved in ethyl acetate (5.00 mL), and hydrochloride ethyl acetate solution (4 mol/L, 893 μL) was added thereto, and the reaction mixture was stirred and reacted for 2 hours at 25° C. After the reaction mixture was concentrated under reduced pressure, the residue was purified by preparative high performance liquid chromatography (chromatographic column: Xtimate C18 150*40 mm*5 μm, mobile phase A: hydrochloric acid aqueous solution with a volume fraction of 0.05%; mobile phase B: acetonitrile; B %: 16% to 46%, 10 minutes) to obtain the hydrochloride of compound 36. ¹H NMR (400 MHz, CD₃OD) δ=8.58 (s, 1H), 7.93 (s, 1H), 7.74-7.61 (m, 3H), 7.43-7.33 (m, 2H), 7.30 (d, J=8.0 Hz, 1H), 4.45 (d, J=12.0 Hz, 1H), 4.34 (s, 3H), 4.20 (d, J=13.6 Hz, 1H), 3.70-3.39 (m, 3H), 2.27-2.20 (m, 1H), 1.97-1.92 (m, 1H), 1.88-1.65 (m, 2H). MS-ESI calculated for [M+H]⁺ 460, found 460.

### Example 37

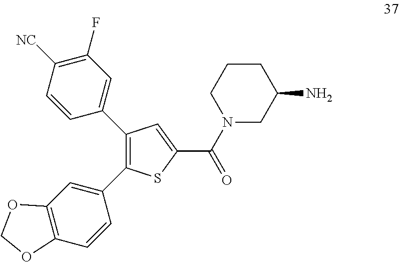 [SMILES: N#Cc1ccc(-c2cc(C(=O)N3CCC[C@@H](N)C3)sc2-c2ccc3c(c2)OCO3)cc1F]

Synthetic Route:

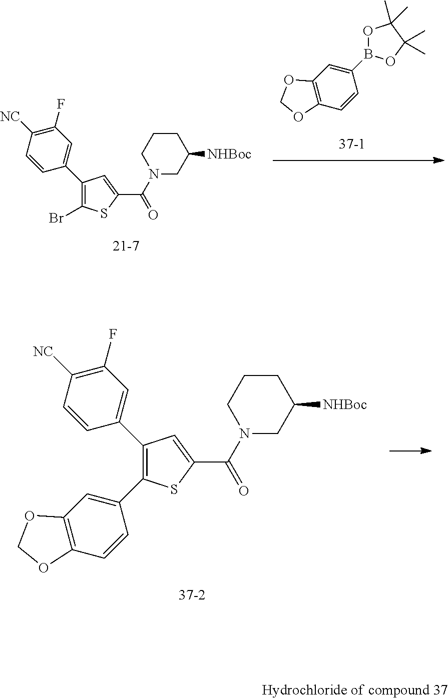 [SMILES: CC(C)(C)OC(=O)N[C@@H]1CCCN(C(=O)c2cc(-c3ccc(C#N)c(F)c3)c(Br)s2)C1 ; CC1(C)OB(c2ccc3c(c2)OCO3)OC1(C)C ; CC(C)(C)OC(=O)N[C@@H]1CCCN(C(=O)c2cc(-c3ccc(C#N)c(F)c3)c(-c3ccc4c(c3)OCO4)s2)C1]

### Step 1

Under nitrogen atmosphere, compound 21-7 (86.4 mg, 166 μmol), compound 37-1 (50.0 mg, 568 μmol), and sodium carbonate (35.2 mg, 333 μmol) were dissolved in dioxane (8.00 mL) and water (2.00 mL), and the reaction mixture was added with [1,1′-bis(diphenylphosphino)ferrocene]dichloropalladium(II) (12.2 mg, 16.7 μmol), and the reaction mixture was stirred and reacted for 12 hours at 100° C. The reaction mixture was concentrated under reduced pressure, and the residue was purified by silica gel column chromatography (1/1, petroleum ether/ethyl acetate, R_{f}=0.38) to obtain compound 37-2. MS-ESI calculated for [M−56+H]⁺ 494, found 494.

### Step 2

Compound 37-2 (62.0 mg, 113 μmol) was dissolved in ethyl acetate (10.0 mL), and hydrochloride ethyl acetate solution (4 mol/L, 282 μL) was added thereto, and the reaction mixture was stirred and reacted for 12 hours at 25° C. After the reaction mixture was concentrated under reduced pressure, the residue was purified by preparative high performance liquid chromatography (chromatographic column: Xtimate C18 150*40 mm*5 μm, mobile phase A: hydrochloric acid aqueous solution with a volume fraction of 0.05%; mobile phase B: acetonitrile; B %: 16% to 46%, 10 minutes) to obtain the hydrochloride of compound 37. ¹H NMR (400 MHz, CD₃OD) δ=7.71 (t, J=8.0 Hz, 1H), 7.57 (s, 1H), 7.33 (dd, J=1.6, 10.4 Hz, 1H), 7.29 (dd, J=1.2, 8.0 Hz, 1H), 6.91-6.81 (m, 2H), 6.75 (d, J=1.6 Hz, 1H), 6.02 (s, 2H), 4.39 (d, J=12.4 Hz, 1H), 4.16 (d, J=13.6 Hz, 1H), 3.65-3.38 (m, 3H), 2.29-2.16 (m, 1H), 1.99-1.88 (m, 1H), 1.82-1.60 (m, 2H). MS-ESI calculated for [M+H]⁺ 450, found 450.

### Example 38

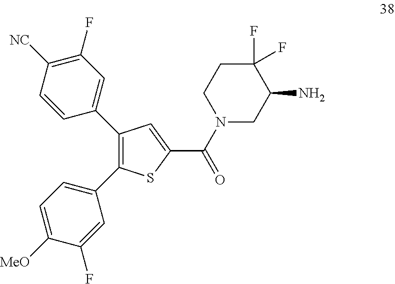 [SMILES: COc1ccc(-c2sc(C(=O)N3CCC(F)(F)[C@@H](N)C3)cc2-c2ccc(C#N)c(F)c2)cc1F]

Synthetic Route:

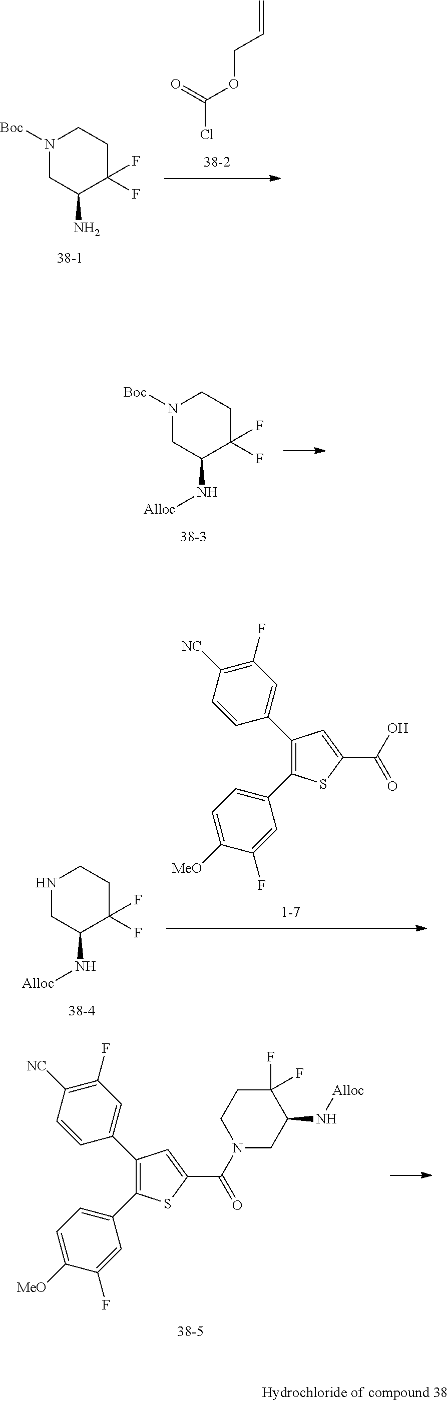

### Step 1

Under nitrogen atmosphere, compound 38-1 (100 mg, 0.423 mmol) was dissolved in anhydrous tetrahydrofuran (10.0 mL), and then sodium bicarbonate (178 mg, 2.12 mmol) and allyl chloroformate (38-2) (56.1 mg, 0.466 mmol) were added thereto, and the reaction mixture was stirred for 12 hours at 25° C., added with water (20 mL), and extracted with ethyl acetate (20 mL×1). The organic phase was washed with saturated brine (20 mL×1), dried over anhydrous sodium sulfate, filtered, and concentrated under reduced pressure. The residue was purified by silica gel column chromatography (2/1, petroleum ether/ethyl acetate, R_{f}=0.70) to obtain compound 38-3. ¹H NMR (400 MHz, CDCl₃) δ 6.01-5.88 (m, 1H), 5.38-5.22 (m, 2H), 5.01-4.91 (m, 1H), 4.52-4.54 (m, 2H), 4.20-4.00 (m, 2H), 3.98-3.90 (m, 1H), 3.20-3.10 (m, 1H), 3.06-2.94 (m, 1H), 2.19-2.08 (m, 1H), 2.02-1.86 (m, 1H), 1.48 (s, 9H).

### Step 2

Compound 38-3 (125 mg, 0.390 mmol) was dissolved in ethyl acetate (5.00 mL), and hydrochloride ethyl acetate solution (4 mol/L, 5.00 mL) was added dropwise thereto at 25° C. The reaction mixture was stirred for 2 hours at 25° C. and concentrated under reduced pressure to obtain crude compound 38-4. ¹H NMR (400 MHz, CD₃OD) δ 6.00-5.89 (m, 1H), 5.36-5.29 (m, 1H), 5.21 (dd, J=1.6, 10.4 Hz, 1H), 4.63-4.55 (m, 2H), 4.42-4.28 (m, 1H), 3.58-3.49 (m, 2H), 3.27-3.16 (m, 1H), 3.11 (t, J=12.4 Hz, 1H), 2.56-2.44 (m, 1H), 2.38-2.18 (m, 1H).

### Step 3

Under nitrogen atmosphere, compound 1-7 (140 mg, 0.377 mmol) was dissolved in anhydrous N,N-dimethylformamide (5.00 mL), and then triethylamine (191 mg, 1.88 mmol), compound 38-4 (99.6 mg, 0.452 mmol), and 2-(7-azabenzotriazol-1-yl)-N,N,N′,N′-tetramethyluronium hexafluorophosphate (215 mg, 0.565 mmol) were added thereto. The reaction mixture was stirred for 12 hours at 25° C. The reaction mixture was added with water (20 mL), and extracted with dichloromethane (20 mL×1). The organic phase was washed with saturated brine (20 mL×1), dried over anhydrous sodium sulfate, filtered, and concentrated under reduced pressure, and the residue was purified by silica gel column chromatography (1/1, petroleum ether/ethyl acetate, R_{f}=0.51) to obtain compound 38-5. MS-ESI calculated for [M+H]⁺ 574, found 574.

### Step 4

Under nitrogen atmosphere, compound 38-5 (130 mg, 0.227 mmol) was dissolved in anhydrous tetrahydrofuran (5 mL), and then ethylenediamine (111 mg, 1.52 mmol) and tetrakis(triphenylphosphine)palladium (26.2 mg, 0.227 mmol) were added thereto, and the reaction mixture was stirred for 12 hours at 65° C., and concentrated under reduced pressure. The residue was purified by silica gel column chromatography (10/1, dichloromethane/methanol, R_{f}=0.41), and then separated by preparative high performance liquid chromatography (chromatographic column: Phenomenex Genimi NX C18 150*40 mm*5 μm; mobile phase: mobile phase A: ammonia aqueous solution with a volume fraction of 0.04% and 10 mmol/L sodium bicarbonate aqueous solution; mobile phase B: acetonitrile; B %: 35% to 65%, 10 min) to obtain the compound, which was added with hydrochloric acid aqueous solution (4 mol/L, 2 mL) to obtain the hydrochloride of compound 38. ¹H NMR (400 MHz, CD₃OD) δ 7.74-7.67 (m, 1H), 7.63 (s, 1H), 7.35 (dd, J=1.2, 10.4 Hz, 1H), 7.26 (dd, J=1.6, 8.0 Hz, 1H), 7.16-7.03 (m, 3H), 4.70-4.62 (m, 1H), 4.50-4.42 (m, 1H), 4.00-3.91 (m, 1H), 3.90 (s, 3H), 3.63-3.55 (m, 1H), 3.53-3.45 (m, 1H), 2.42-2.22 (m, 2H). MS-ESI calculated for [M+H]⁺ 490, found 490.

### Example 39

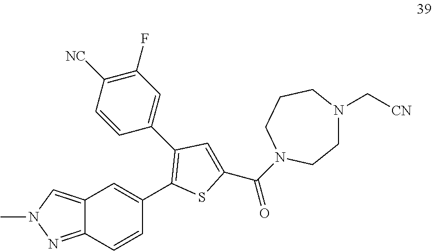 [SMILES: Cn1cc2cc(-c3sc(C(=O)N4CCCN(CC#N)CC4)cc3-c3ccc(C#N)c(F)c3)ccc2n1]

Synthetic Route:

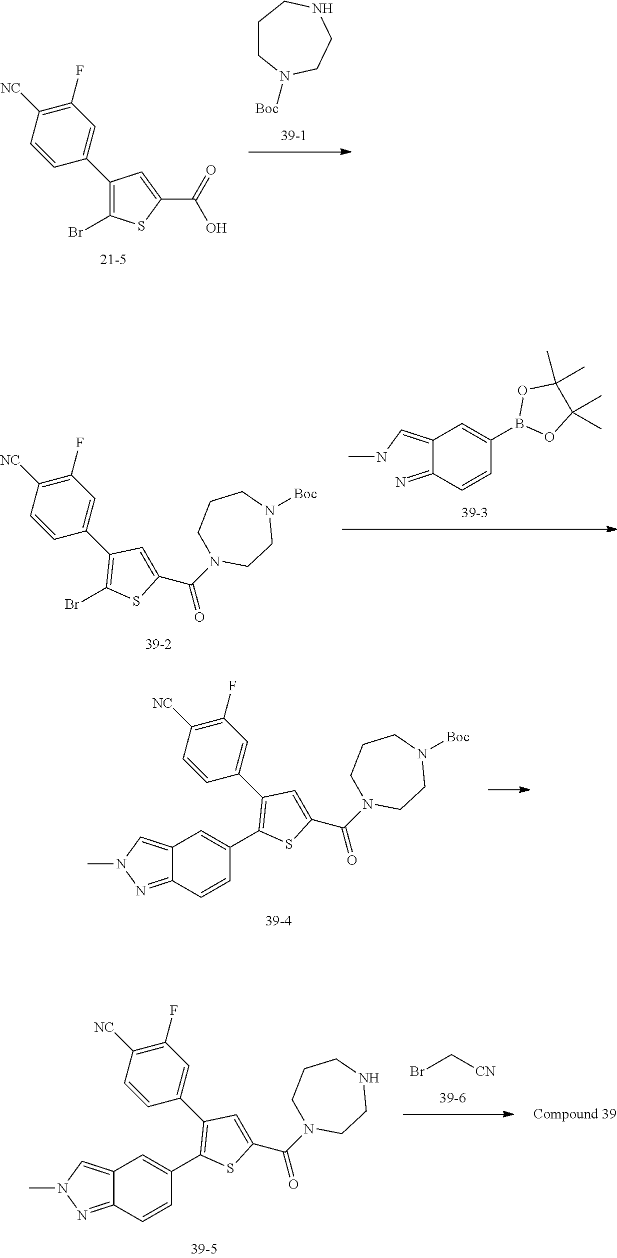

### Step 1

Under nitrogen atmosphere, compound 21-5 (160 mg, 0.491 mmol) was dissolved in anhydrous dichloromethane (10.0 mL), and then triethylamine (149 mg, 1.47 mmol), 1-boc-hexahydro-1,4-diazepine (118 mg, 0.589 mmol), and 2-(7-azabenzotriazol-1-yl)-N,N,N′,N′-tetramethyluronium hexafluorophosphate (280 mg, 0.736 mmol) were added thereto. The reaction mixture was stirred for 2 hours at 25° C. and concentrated under reduced pressure. The residue was purified by silica gel column chromatography (1/1, petroleum ether/ethyl acetate, R_{f}=0.34) to obtain compound 39-2. MS-ESI calculated for [M+H]⁺ 509, found 509.

### Step 2

Under nitrogen atmosphere, compound 39-2 (180 mg, 0.354 mmol) was dissolved in 1,4-dioxane (10.0 mL) and water (2.00 mL), and the reaction mixture was added with 2-methyl-5-(tetramethyl-1,3,2-dioxaborolan-2-yl)-2H-indazole (91.4 mg, 0.354 mmol), potassium phosphate (225 mg, 1.06 mmol), and [1,1′-bis(diphenylphosphino)ferrocene]dichloropalladium(II) (25.9 mg, 0.035 mmol), stirred for 12 hours at 100° C., added with water (20 mL), and extracted with ethyl acetate (20 mL×1). The organic phase was washed with saturated brine (20 mL×3), dried over anhydrous sodium sulfate, filtered, and concentrated under reduced pressure. The residue was purified by silica gel column chromatography (1/1, petroleum ether/ethyl acetate, R_{f}=0.07) to obtain compound 39-4. MS-ESI calculated for [M+H]⁺ 560, found 560.

### Step 3

Compound 39-4 (110 mg, 0.197 mmol) was dissolved in ethyl acetate (5.00 mL), and hydrochloride ethyl acetate solution (4 mol/L, 5.00 mL) was added dropwise thereto at 25° C. The reaction mixture was stirred for 12 hours at 25° C. and concentrated under reduced pressure to obtain compound 39-5. MS-ESI calculated for [M+H]⁺ 460, found 460.

### Step 4

Under nitrogen atmosphere, compound 39-5 (80.0 mg, 0.161 mmol) was dissolved in anhydrous N,N-dimethylformamide (3.00 mL), and then potassium carbonate (66.9 mg, 0.484 mmol) and bromoacetonitrile (29.0 mg, 0.242 mmol) were added thereto, and the reaction mixture was stirred for 12 hours at 25° C., filtered, and the residue was separated by preparative high performance liquid chromatography (chromatographic column: Phenomenex Genimi NX C18 150*40 mm*5 μm; mobile phase: mobile phase A: ammonia aqueous solution with a volume fraction of 0.04% and 10 mmol/L sodium bicarbonate aqueous solution; mobile phase B: acetonitrile; B %: 30 to 60%, 10 minutes) to obtain compound 39. ¹H NMR (400 MHz, CD₃OD) δ 8.40 (s, 1H), 7.87-7.77 (m, 2H), 7.73-7.62 (m, 1H), 7.61-7.52 (m, 2H), 7.27-7.21 (m, 1H), 7.05-6.99 (m, 1H), 4.18 (s, 3H), 4.01-3.85 (br s, 2H), 3.81 (s, 2H), 3.76-3.58 (m, 2H), 2.92-2.75 (m, 2H), 2.72-2.64 (m, 2H), 2.07-1.71 (m, 2H). MS-ESI calculated for [M+H]⁺ 499, found 499.

### Example 40

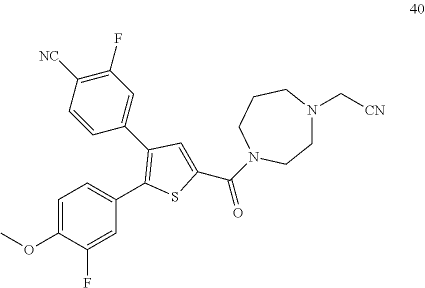 [SMILES: COc1ccc(-c2sc(C(=O)N3CCCN(CC#N)CC3)cc2-c2ccc(C#N)c(F)c2)cc1F]

Synthetic Route:

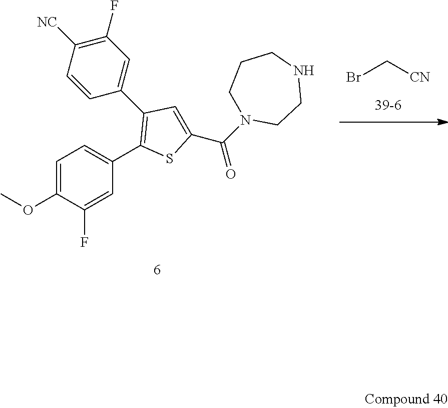

Under nitrogen atmosphere, compound 6 (50.0 mg, 0.102 mmol) was dissolved in anhydrous N,N-dimethylformamide (3.00 mL), and then potassium carbonate (42.3 mg, 0.306 mmol) and bromoacetonitrile (13.5 mg, 0.112 mmol) were added thereto, and the reaction mixture was stirred for 12 hours at 25° C., filtered, and the residue was separated by preparative high performance liquid chromatography (chromatographic column: Phenomenex Genimi NX C18 150*40 mm*5 μm; mobile phase: mobile phase A: 10 mmol/L sodium bicarbonate aqueous solution; mobile phase B: acetonitrile; B %: 43 to 73%, 9 minutes) to obtain compound 40. ¹H NMR (400 MHz, CD₃OD) δ 7.72-7.64 (m, 1H), 7.58-7.49 (m, 1H), 7.30 (dd, J=1.6, 10.4 Hz, 1H), 7.24 (dd, J=1.6, 8.0 Hz, 1H), 7.13-7.00 (m, 3H), 4.04-3.90 (m, 2H), 3.89 (s, 3H), 3.88-3.76 (m, 2H), 3.75 (s, 2H), 2.96-2.89 (m, 2H), 2.85-2.74 (m, 2H), 2.09-1.95 (m, 2H). MS-ESI calculated for [M+H]⁺ 493, found 493.

### Example 41

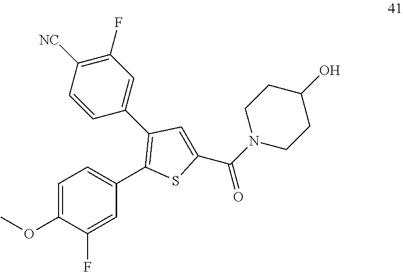 [SMILES: COc1ccc(-c2sc(C(=O)N3CCC(O)CC3)cc2-c2ccc(C#N)c(F)c2)cc1F]

Synthetic Route:

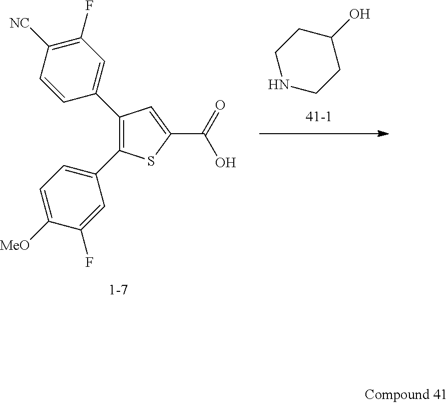 [SMILES: COc1ccc(-c2sc(C(=O)O)cc2-c2ccc(C#N)c(F)c2)cc1F ; OC1CCNCC1]

Under nitrogen atmosphere, compound 1-7 (50.0 mg, 0.135 mmol) was dissolved in anhydrous N,N-dimethylformamide (3.00 mL), and then triethylamine (40.9 mg, 0.404 mmol), 4-hydroxypiperidine (41-1) (16.3 mg, 0.162 mmol), and 2-(7-azabenzotriazol-1-yl)-N,N,N′,N′-tetramethyluronium hexafluorophosphate (76.8 mg, 0.202 mmol) were added thereto. The reaction mixture was stirred for 12 hours at 25° C., and filtered. The residue was separated by preparative high performance liquid chromatography (chromatographic column: Xtimate C18 150*40 mm*5 μm; mobile phase: mobile phase A: hydrochloric acid aqueous solution with a volume fraction of 0.05%; mobile phase B: acetonitrile; B %: 35 to 65%, 10 minutes) to obtain compound 41. ¹H NMR (400 MHz, CD₃OD) δ 7.72-7.65 (m, 1H), 7.49 (s, 1H), 7.31 (dd, J=1.6, 10.4 Hz, 1H), 7.25 (dd, J=1.6, 8.0 Hz, 1H), 7.14-7.02 (m, 3H), 4.21-4.10 (m, 2H), 3.98-3.91 (m, 1H), 3.90 (s, 3H), 3.57-3.41 (m, 2H), 2.01-1.89 (m, 2H), 1.64-1.50 (m, 2H). MS-ESI calculated for [M+H]⁺ 455, found 455.

### Example 42

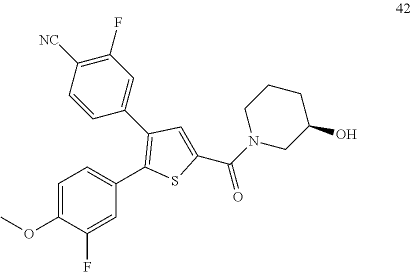 [SMILES: COc1ccc(-c2sc(C(=O)N3CCC[C@@H](O)C3)cc2-c2ccc(C#N)c(F)c2)cc1F]

Synthetic Route:

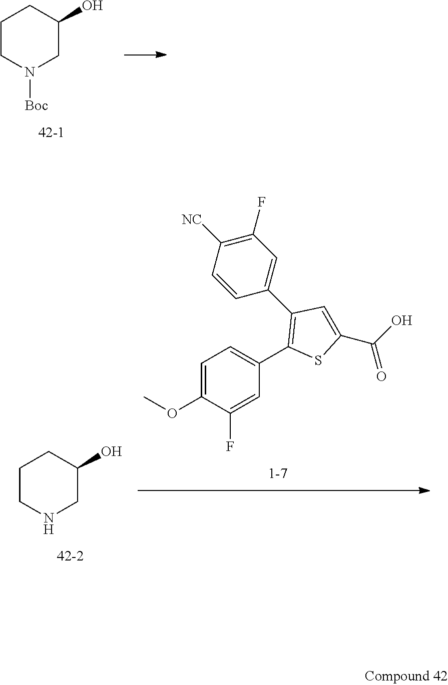 [SMILES: CC(C)(C)OC(=O)N1CCC[C@@H](O)C1 ; COc1ccc(-c2sc(C(=O)O)cc2-c2ccc(C#N)c(F)c2)cc1F ; O[C@@H]1CCCNC1]

### Step 1

Compound 42-1 (1.00 g, 0.497 mmol) was dissolved in ethyl acetate (10.0 mL), and hydrochloride ethyl acetate solution (4 mol/L, 10 mL) was added dropwise thereto at 25° C. The reaction mixture was stirred for 12 hours at 25° C. and concentrated under reduced pressure to obtain crude compound 42-2. ¹H NMR (400 MHz, CDOD₃) δ 4.10-3.94 (m, 1H), 3.23-3.00 (m, 4H), 2.16-2.03 (m, 1H), 1.93-1.80 (m, 1H), 1.79-1.65 (m, 2H).

### Step 2

Under nitrogen atmosphere, compound 1-7 (50.0 mg, 0.135 mmol) was dissolved in anhydrous N,N-dimethylformamide (3.00 mL), and then triethylamine (40.9 mg, 0.404 mmol), compound 42-2 (22.2 mg, 0.162 mmol), and 2-(7-azabenzotriazol-1-yl)-N,N,N′,N′-tetramethyluronium hexafluorophosphate (76.8 mg, 0.202 mmol) were added thereto. The reaction mixture was stirred for 12 hours at 25° C., and filtered. The residue was separated by preparative high performance liquid chromatography (chromatographic column: Xtimate C18 150*40 mm*5 μm; mobile phase: mobile phase A: hydrochloric acid aqueous solution with a volume fraction of 0.05%; mobile phase B: acetonitrile; B %: 35 to 65%, 10 minutes) to obtain compound 42. ¹H NMR (400 MHz, CD₃OD) δ 7.70-7.64 (m, 1H), 7.52 (s, 1H), 7.29 (dd, J=1.6, 10.4 Hz, 1H), 7.24 (dd, J=1.6, 8.0 Hz, 1H), 7.12-7.00 (m, 3H), 4.09-3.94 (m, 1H), 3.89 (s, 3H), 3.87-3.73 (m, 2H), 3.65-3.34 (m, 2H), 2.03-1.85 (m, 2H), 1.72-1.53 (m, 2H). MS-ESI calculated for [M+H]⁺ 455, found 455.

### Example 43

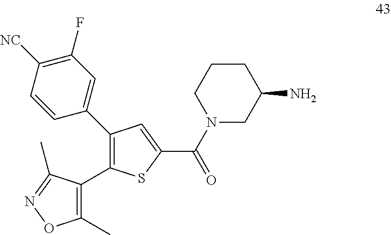 [SMILES: Cc1noc(C)c1-c1sc(C(=O)N2CCC[C@@H](N)C2)cc1-c1ccc(C#N)c(F)c1]

Synthetic Route:

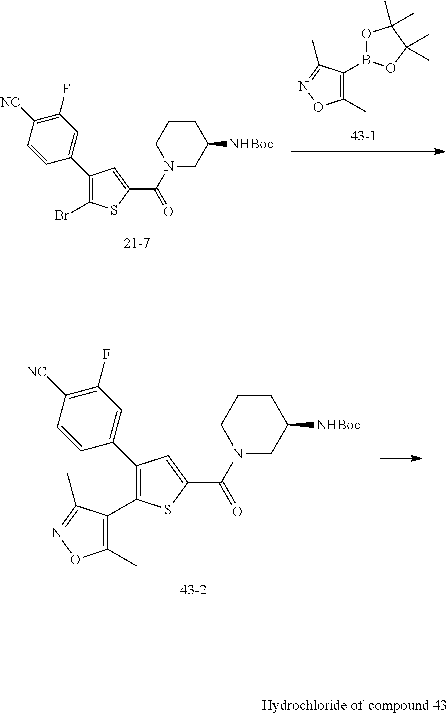 [SMILES: CC(C)(C)OC(=O)N[C@@H]1CCCN(C(=O)c2cc(-c3ccc(C#N)c(F)c3)c(Br)s2)C1 ; Cc1noc(C)c1B1OC(C)(C)C(C)(C)O1 ; Cc1noc(C)c1-c1sc(C(=O)N2CCC[C@@H](NC(=O)OC(C)(C)C)C2)cc1-c1ccc(C#N)c(F)c1]

### Step 1

Under nitrogen atmosphere, compound 21-7 (190 mg, 374 μmol), compound 43-1 (100 mg, 448 μmol), and sodium carbonate (79.2 mg, 747 μmol) were dissolved in dioxane (8.00 mL) and water (2.00 mL), and the reaction mixture was added with [1,1′-bis(diphenylphosphino)ferrocene]dichloropalladium(II) (27.3 mg, 37.4 μmol), and the reaction mixture was stirred and reacted for 12 hours at 100° C. The reaction mixture was concentrated under reduced pressure, and the residue was purified by silica gel column chromatography (1/1, petroleum ether/ethyl acetate, R_{f}=0.55) to obtain compound 43-2. MS-ESI calculated for [M+H]⁺ 525, found 525.

### Step 2

Compound 43-2 (140 mg, 267 μmol) was dissolved in ethyl acetate (20.0 mL), and hydrochloride ethyl acetate solution (4 mol/L, 667 μL) was added thereto, and the reaction mixture was stirred and reacted for 12 hours at 25° C. After the reaction mixture was concentrated, the residue was purified by preparative high performance liquid chromatography (chromatographic column: Xtimate C18 150*40 mm*5 μm, mobile phase A: hydrochloric acid aqueous solution with a volume fraction of 0.05%; mobile phase B: acetonitrile; B %: 15% to 45%, 10 minutes) to obtain the hydrochloride of compound 43. ¹H NMR (400 MHz, CD₃OD) δ=7.81-7.74 (m, 1H), 7.71 (s, 1H), 7.41 (dd, J=1.2, 10.3 Hz, 1H), 7.29 (dd, J=1.6, 8.0 Hz, 1H), 4.39 (d, J=10.4 Hz, 1H), 4.16 (d, J=12.8 Hz, 1H), 3.67-3.39 (m, 3H), 2.25 (s, 3H), 2.23-2.15 (m, 1H), 1.99 (s, 3H), 1.94-1.86 (m, 1H), 1.84-1.71 (m, 2H). MS-ESI calculated for [M+H]⁺ 425, found 425.

### Example 44

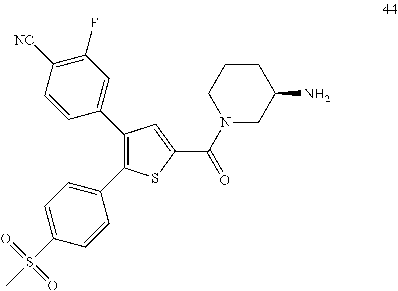 [SMILES: CS(=O)(=O)c1ccc(-c2sc(C(=O)N3CCC[C@@H](N)C3)cc2-c2ccc(C#N)c(F)c2)cc1]

Synthetic Route:

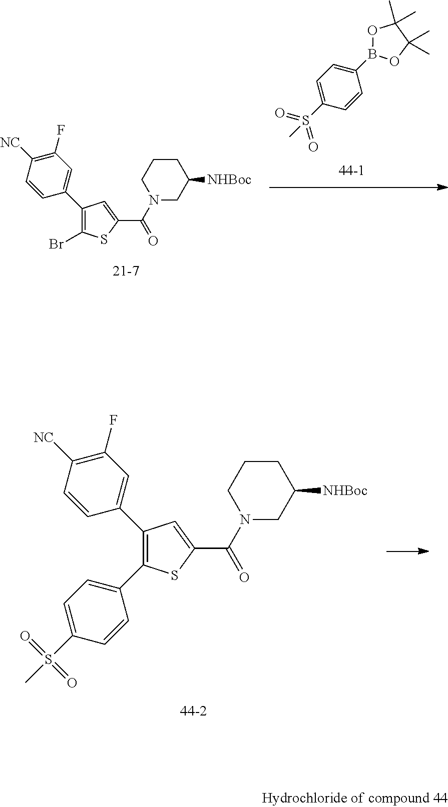 [SMILES: CC(C)(C)OC(=O)N[C@@H]1CCCN(C(=O)c2cc(-c3ccc(C#N)c(F)c3)c(Br)s2)C1 ; CC1(C)OB(c2ccc(S(C)(=O)=O)cc2)OC1(C)C ; CC(C)(C)OC(=O)N[C@@H]1CCCN(C(=O)c2cc(-c3ccc(C#N)c(F)c3)c(-c3ccc(S(C)(=O)=O)cc3)s2)C1]

### Step 1

Under nitrogen atmosphere, compound 21-7 (75.1 mg, 148 μmol), compound 44-1 (50.0 mg, 177 μmol), and sodium carbonate (31.3 mg, 295 μmol) were dissolved in dioxane (8.00 mL) and water (2.00 mL), and the reaction mixture was added with [1,1′-bis(diphenylphosphino)ferrocene]dichloropalladium(II) (10.8 mg, 14.8 μmol), and the reaction mixture was stirred and reacted for 12 hours at 100° C. The reaction mixture was concentrated under reduced pressure, and the residue was purified by silica gel column chromatography (1/1, petroleum ether/ethyl acetate, R_{f}=0.25) to obtain compound 44-2. MS-ESI calculated for [M+H]⁺ 584, found 584.

### Step 2

Compound 44-2 (75.0 mg, 128 μmol) was dissolved in ethyl acetate (10.0 mL), and hydrochloride ethyl acetate solution (4 mol/L, 321 μL) was added thereto, and the reaction mixture was stirred and reacted for 12 hours at 25° C. After the reaction mixture was concentrated, the residue was purified by preparative high performance liquid chromatography (chromatographic column: Xtimate C18 150*40 mm*5 μm, mobile phase A: hydrochloric acid aqueous solution with a volume fraction of 0.05%; mobile phase B: acetonitrile; B %: 10% to 40%, 10 minutes) to obtain the hydrochloride of compound 44. ¹H NMR (400 MHz, CD₃OD) δ=7.98 (d, J=8.4 Hz, 2H), 7.73 (t, J=7.4 Hz, 1H), 7.67-7.54 (m, 3H), 7.40 (d, J=10.4 Hz, 1H), 7.26 (d, J=8.0 Hz, 1H), 4.40 (d, J=12.4 Hz, 1H), 4.16 (d, J=13.2 Hz, 1H), 3.67-3.42 (m, 3H), 3.17 (s, 3H), 2.28-2.15 (m, 1H), 1.99-1.88 (m, 1H), 1.83-1.69 (m, 2H). MS-ESI calculated for [M+H]⁺ 484, found 484.

### Example 45

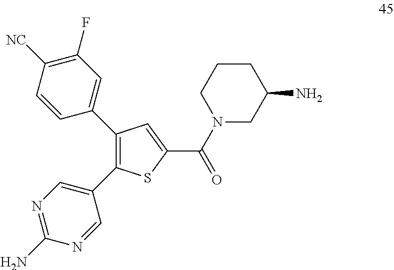 [SMILES: N#Cc1ccc(-c2cc(C(=O)N3CCC[C@@H](N)C3)sc2-c2cnc(N)nc2)cc1F]

Synthetic Route:

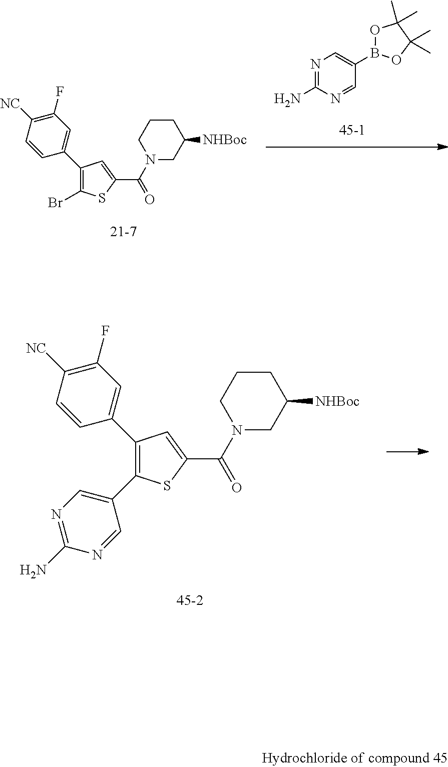 [SMILES: CC(C)(C)OC(=O)N[C@@H]1CCCN(C(=O)c2cc(-c3ccc(C#N)c(F)c3)c(Br)s2)C1 ; CC1(C)OB(c2cnc(N)nc2)OC1(C)C ; CC(C)(C)OC(=O)N[C@@H]1CCCN(C(=O)c2cc(-c3ccc(C#N)c(F)c3)c(-c3cnc(N)nc3)s2)C1]

### Step 1

Under nitrogen atmosphere, compound 21-7 (100 mg, 197 μmol), compound 45-1 (52.2 mg, 236 μmol), and sodium carbonate (41.7 mg, 393 μmol) were dissolved in dioxane (8.00 mL) and water (2.00 mL), and the reaction mixture was added with [1,1′-bis(diphenylphosphino)ferrocene]dichloropalladium(II) (14.4 mg, 19.7 μmol), and the reaction mixture was stirred and reacted for 12 hours at 100° C. The reaction mixture was concentrated under reduced pressure, and the residue was purified by silica gel column chromatography (1/1, petroleum ether/ethyl acetate, R_{f}=0.07) to obtain compound 45-2. MS-ESI calculated for [M+Na]⁺ 545, found 545.

### Step 2

Compound 45-2 (54.0 mg, 103 μmol) was dissolved in ethyl acetate (10.00 mL), and hydrochloride ethyl acetate solution (4 mol/L, 258 μL) was added thereto, and the reaction mixture was stirred and reacted for 12 hours at 25° C. After the reaction mixture was concentrated, the residue was purified by preparative high performance liquid chromatography (chromatographic column: Xtimate C18 150*40 mm*5 μm, mobile phase A: hydrochloric acid aqueous solution with a volume fraction of 0.05%; mobile phase B: acetonitrile; B %: 1% to 30%, 10 minutes) to obtain the hydrochloride of compound 45. ¹H NMR (400 MHz, CD₃OD) δ=8.51 (s, 2H), 7.81 (t, J=8.0 Hz, 1H), 7.66 (s, 1H), 7.55 (d, J=8.0 Hz, 1H), 7.42 (d, J=8.0 Hz, 1H), 4.43 (d, J=12.0 Hz, 1H), 4.20-4.09 (m, 1H), 3.58-3.41 (m, 3H), 2.28-2.17 (m, 1H), 1.97-1.89 (m, 1H), 1.85-1.72 (m, 2H). MS-ESI calculated for [M+H]⁺ 423, found 423.

### Example 46

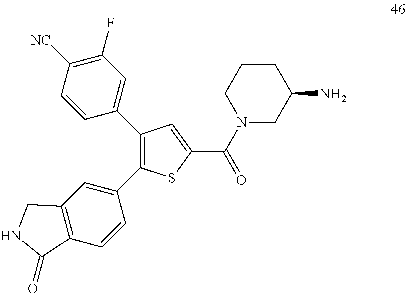 [SMILES: N#Cc1ccc(-c2cc(C(=O)N3CCC[C@@H](N)C3)sc2-c2ccc3c(c2)CNC3=O)cc1F]

Synthetic Route:

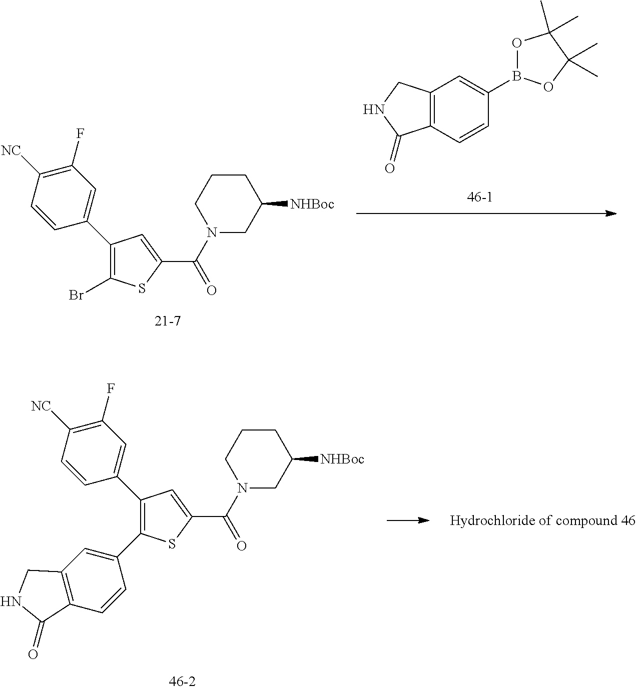 [SMILES: CC(C)(C)OC(=O)N[C@@H]1CCCN(C(=O)c2cc(-c3ccc(C#N)c(F)c3)c(Br)s2)C1 ; CC1(C)OB(c2ccc3c(c2)CNC3=O)OC1(C)C ; CC(C)(C)OC(=O)N[C@@H]1CCCN(C(=O)c2cc(-c3ccc(C#N)c(F)c3)c(-c3ccc4c(c3)CNC4=O)s2)C1]

### Step 1

Under nitrogen atmosphere, compound 21-7 (100 mg, 197 μmol), compound 46-1 (61.2 mg, 236 μmol), and sodium carbonate (41.7 mg, 393 μmol) were dissolved in dioxane (4.00 mL) and water (1.00 mL), and the reaction mixture was added with [1,1′-bis(diphenylphosphino)ferrocene]dichloropalladium(II) (14.4 mg, 19.7 μmol), and the reaction mixture was stirred and reacted for 12 hours at 100° C. The reaction mixture was concentrated under reduced pressure, and the residue was purified by silica gel column chromatography (1/1, petroleum ether/ethyl acetate, R_{f}=0.06) to obtain compound 46-2. MS-ESI calculated for [M+H]⁺ 561, found 561.

### Step 2

Compound 46-2 (50.0 mg, 89.2 μmol) was dissolved in ethyl acetate (5.00 mL), and hydrochloride ethyl acetate solution (4 mol/L, 223 μL) was added thereto, and the reaction mixture was stirred and reacted for 12 hours at 25° C. After the reaction mixture was concentrated under reduced pressure, the residue was purified by preparative high performance liquid chromatography (chromatographic column: Xtimate C18 150*40 mm*5 μm, mobile phase A: hydrochloric acid aqueous solution with a volume fraction of 0.05%; mobile phase B: acetonitrile; B %: 5% to 35%, 10 minutes) to obtain the hydrochloride of compound 46. ¹H NMR (400 MHz, CD₃OD) δ=7.78 (d, J=8.0 Hz, 1H), 7.68 (t, J=8.0 Hz, 1H), 7.62 (s, 1H), 7.58 (s, 1H), 7.44 (d, J=8.0 Hz, 1H), 7.36 (d, J=8.0 Hz, 1H), 7.23 (d, J=8.0 Hz, 1H), 4.45 (s, 2H), 4.38 (d, J=12.0 Hz, 1H), 4.15 (d, J=12.0 Hz, 1H), 3.59-3.38 (m, 3H), 2.24-2.17 (m, 1H), 1.94-1.86 (m, 1H), 1.81-1.70 (m, 2H). MS-ESI calculated for [M+H]⁺ 461, found 461.

### Example 47

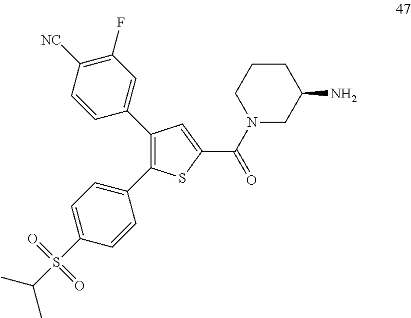 [SMILES: CC(C)S(=O)(=O)c1ccc(-c2sc(C(=O)N3CCC[C@@H](N)C3)cc2-c2ccc(C#N)c(F)c2)cc1]

Synthetic Route:

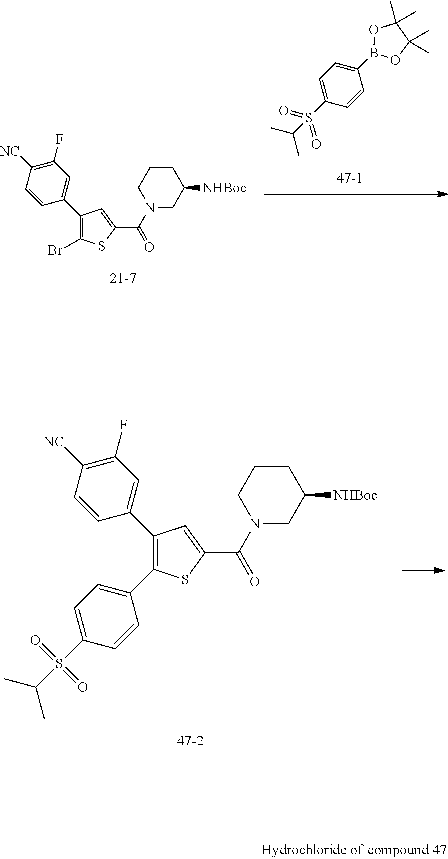 [SMILES: CC(C)(C)OC(=O)N[C@@H]1CCCN(C(=O)c2cc(-c3ccc(C#N)c(F)c3)c(Br)s2)C1 ; CC(C)S(=O)(=O)c1ccc(B2OC(C)(C)C(C)(C)O2)cc1 ; CC(C)S(=O)(=O)c1ccc(-c2sc(C(=O)N3CCC[C@@H](NC(=O)OC(C)(C)C)C3)cc2-c2ccc(C#N)c(F)c2)cc1]

### Step 1

Under nitrogen atmosphere, compound 21-7 (163 mg, 322 μmol), compound 47-1 (100 mg, 322 μmol), and sodium carbonate (68.3 mg, 644 μmol) were dissolved in dioxane (32.0 mL) and water (4.00 mL), and the reaction mixture was added with [1,1′-bis(diphenylphosphino)ferrocene]dichloropalladium(II) (23.6 mg, 32.2 μmol), and the reaction mixture was stirred and reacted for 12 hours at 100° C. The reaction mixture was concentrated under reduced pressure, and the residue was purified by silica gel column chromatography (1/2, petroleum ether/ethyl acetate, R_{f}=0.45) to obtain compound 47-2. MS-ESI calculated for [M+H]⁺ 612, found 612.

### Step 2

Compound 47-2 (140 mg, 229 μmol) was dissolved in ethyl acetate (20.0 mL), and hydrochloride ethyl acetate solution (4 mol/L, 572 μL) was added thereto, and the reaction mixture was stirred and reacted for 24 hours at 25° C. After the reaction mixture was concentrated under reduced pressure, the residue was purified by preparative high performance liquid chromatography (chromatographic column: Xtimate C18 150*40 mm*5 μm, mobile phase A: hydrochloric acid aqueous solution with a volume fraction of 0.05%; mobile phase B: acetonitrile; B %: 15% to 45%, 10 minutes) to obtain the hydrochloride of compound 47. ¹H NMR (400 MHz, CD₃OD) δ=7.91 (d, J=8.4 Hz, 2H), 7.73 (t, J=7.6 Hz, 1H), 7.67-7.57 (m, 3H), 7.37 (d, J=10.4 Hz, 1H), 7.27 (dd, J=1.2, 8.0 Hz, 1H), 4.40 (d, J=11.2 Hz, 1H), 4.16 (d, J=13.2 Hz, 1H), 3.60-3.36 (m, 4H), 2.26-2.13 (m, 1H), 1.97-1.91 (m, 1H), 1.85-1.69 (m, 2H), 1.29 (s, 3H), 1.28 (s, 3H). MS-ESI calculated for [M+H]⁺ 512, found 512.

### Example 48

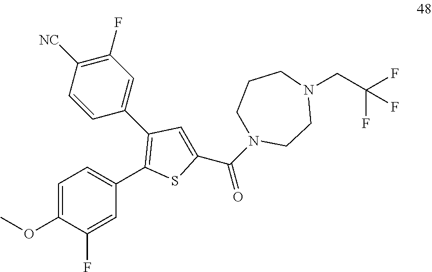 [SMILES: COc1ccc(-c2sc(C(=O)N3CCCN(CC(F)(F)F)CC3)cc2-c2ccc(C#N)c(F)c2)cc1F]

Synthetic Route:

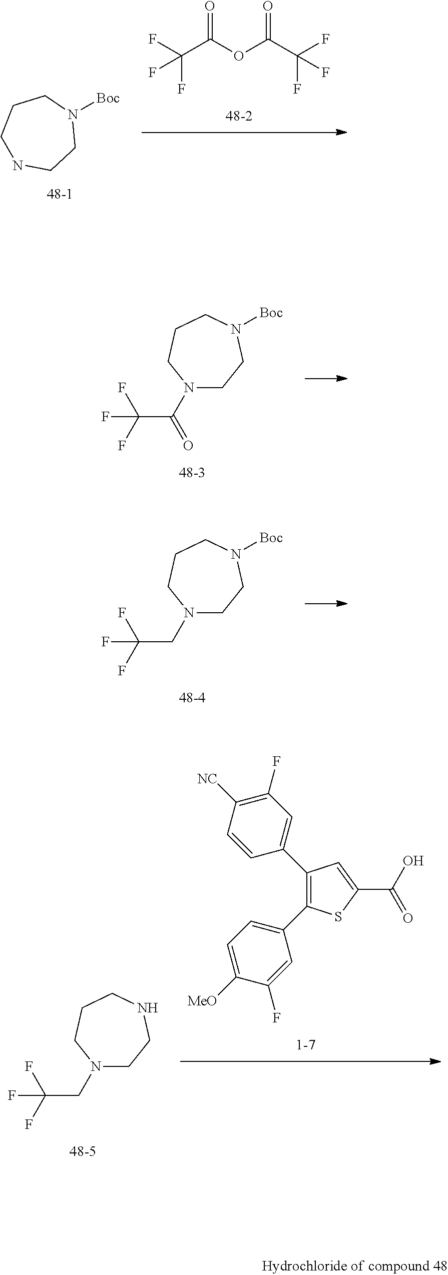 [SMILES: O=C(OC(=O)C(F)(F)F)C(F)(F)F ; CC(C)(C)OC(=O)N1CCCNCC1 ; CC(C)(C)OC(=O)N1CCCN(C(=O)C(F)(F)F)CC1 ; CC(C)(C)OC(=O)N1CCCN(CC(F)(F)F)CC1 ; FC(F)(F)CN1CCCNCC1 ; COc1ccc(-c2sc(C(=O)O)cc2-c2ccc(C#N)c(F)c2)cc1F]

### Step 1

Under nitrogen atmosphere, compound 48-1 (500 mg, 2.50 mmol) was dissolved in anhydrous dichloromethane (10.0 mL), and triethylamine (379 mg, 2.12 mmol) was added thereto. Trifluoromethanesulfonic anhydride (48-2) (629 mg, 3.00 mmol) was added dropwise thereto at 0° C., and the reaction mixture was stirred at 25° C. for 12 hours, added with water (20 mL), and extracted with dichloromethane (20 mL×1). The organic phase was washed with saturated brine (20 mL×1), dried over anhydrous sodium sulfate, filtered, and concentrated under reduced pressure, and the residue was purified by silica gel column chromatography (4/1, petroleum ether/ethyl acetate, R_{f}=0.65) to obtain compound 48-3. ¹H NMR (400 MHz, CDCl₃) δ 3.76-3.53 (m, 6H), 3.52-3.36 (m, 2H), 2.00-1.89 (m, 2H), 1.47 (s, 9H).

### Step 2

Under nitrogen atmosphere, compound 48-3 (250 mg, 0.844 mmol) was dissolved in anhydrous tetrahydrofuran (10.0 mL), and borane-dimethylsulfide (10 mol/L, 422 μL) was added dropwise thereto at 0° C. The reaction mixture was stirred at 65° C. for 12 hours, cooled to 0° C., added with methanol (2 mL), stirred at 65° C. for 1 hour, and then concentrated under reduced pressure. The residue was purified by silica gel column chromatography (4/1, petroleum ether/ethyl acetate, R_{f}=0.30) to obtain compound 48-4. ¹H NMR (400 MHz, CDCl₃) δ 3.53-3.38 (m, 4H), 3.14 (q, J=9.2 Hz, 2H), 2.98-2.83 (m, 4H), 1.88-1.74 (m, 2H), 1.47 (s, 9H).

### Step 3

Compound 48-4 (200 mg, 0.708 mmol) was dissolved in ethyl acetate (5.00 mL), and hydrochloride ethyl acetate solution (4 mol/L, 5 mL) was added dropwise thereto at 25° C. The reaction mixture was stirred for 12 hours at 25° C. and concentrated under reduced pressure to obtain crude compound 48-5. ¹H NMR (400 MHz, CDOD₃) δ 3.70 (q, J=9.2 Hz, 2H), 3.45-3.41 (m, 1H), 3.42 (s, 3H), 3.39-3.34 (m, 2H), 3.27 (t, J=6.0 Hz, 2H), 2.17-2.10 (m, 2H).

### Step 4

Under nitrogen atmosphere, compound 1-7 (45 mg, 0.121 mmol) was dissolved in anhydrous N,N-dimethylformamide (2 mL), and then triethylamine (36.8 mg, 0.364 mmol), compound 48-5 (31.8 mg, 0.145 mmol), and 2-(7-azabenzotriazol-1-yl)-N,N,N′,N′-tetramethyluronium hexafluorophosphate (69.1 mg, 0.182 mmol) were added thereto. The reaction mixture was stirred for 12 hours at 25° C., and concentrated under reduced pressure. The residue was separated by preparative high performance liquid chromatography (chromatographic column: Xtimate C18 150*40 mm*5 μm; mobile phase: mobile phase A: hydrochloric acid aqueous solution with a volume fraction of 0.05%; mobile phase B: acetonitrile; B %: 50% to 80%, 10 minutes) to obtain the hydrochloride of compound 48. ¹H NMR (400 MHz, CD₃OD) δ 7.71-7.64 (m, 1H), 7.57-7.49 (m, 1H), 7.33-7.27 (m, 1H), 7.26-7.22 (m, 1H), 7.14-7.00 (m, 3H), 4.04-3.92 (m, 2H), 3.89 (s, 3H), 3.87-3.71 (m, 2H), 3.54-3.35 (m, 2H), 3.20-3.12 (m, 2H), 3.10-2.97 (m, 2H), 2.09-1.94 (m, 2H). MS-ESI calculated for [M+H]⁺ 536, found 536.

### Example 49

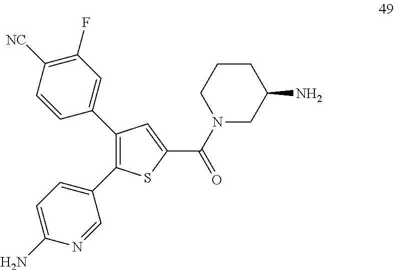 [SMILES: N#Cc1ccc(-c2cc(C(=O)N3CCC[C@@H](N)C3)sc2-c2ccc(N)nc2)cc1F]

Synthetic Route:

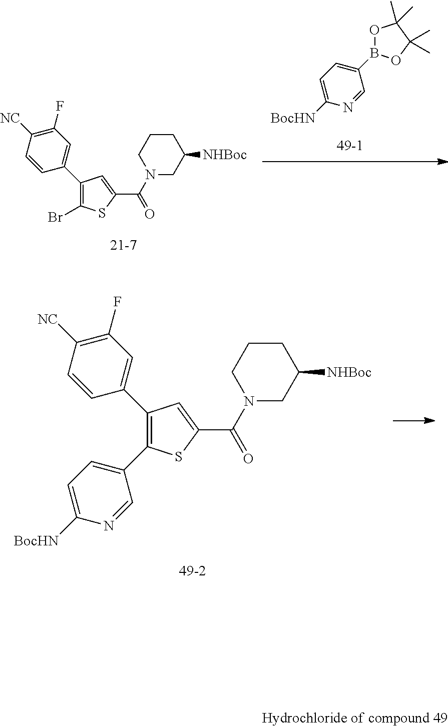 [SMILES: CC(C)(C)OC(=O)N[C@@H]1CCCN(C(=O)c2cc(-c3ccc(C#N)c(F)c3)c(Br)s2)C1 ; CC(C)(C)OC(=O)Nc1ccc(B2OC(C)(C)C(C)(C)O2)cn1 ; CC(C)(C)OC(=O)Nc1ccc(-c2sc(C(=O)N3CCC[C@@H](NC(=O)OC(C)(C)C)C3)cc2-c2ccc(C#N)c(F)c2)cn1]

### Step 1

Under nitrogen atmosphere, compound 21-7 (92.6 mg, 182 μmol), compound 49-1 (70.0 mg, 219 μmol), and sodium carbonate (38.6 mg, 364 μmol) were dissolved in dioxane (16.0 mL) and water (4.00 mL), and the reaction mixture was added with [1,1′-bis(diphenylphosphino)ferrocene]dichloropalladium(II) (13.3 mg, 18.2 μmol), and the reaction mixture was stirred and reacted for 12 hours at 100° C. The reaction mixture was concentrated under reduced pressure, and the residue was purified by silica gel column chromatography (1/1, petroleum ether/ethyl acetate, R_{f}=0.50) to obtain compound 49-2. MS-ESI calculated for [M+H]⁺ 622, found 622.

### Step 2

Compound 49-2 (100 mg, 161 μmol) was dissolved in ethyl acetate (20.0 mL), and hydrochloride ethyl acetate solution (4 mol/L, 402 μL) was added thereto, and the reaction mixture was stirred and reacted for 12 hours at 25° C. After the reaction mixture was concentrated, the residue was purified by preparative high performance liquid chromatography (chromatographic column: Xtimate C18 150*40 mm*5 μm, mobile phase A: hydrochloric acid aqueous solution with a volume fraction of 0.05%; mobile phase B: acetonitrile; B %: 1% to 30%, 10 minutes) to obtain the hydrochloride of compound 49. ¹H NMR (400 MHz, CD₃OD) δ=7.95 (s, 1H), 7.85-7.71 (m, 2H), 7.64 (s, 1H), 7.50 (d, J=10.0 Hz, 1H), 7.39 (d, J=7.6 Hz, 1H), 7.04 (d, J=9.2 Hz, 1H), 4.43 (d, J=11.6 Hz, 1H), 4.16 (d, J=9.6 Hz, 1H), 3.61-3.40 (m, 3H), 2.31-2.13 (m, 1H), 2.01-1.88 (m, 1H), 1.87-1.64 (m, 2H). MS-ESI calculated for [M+H]⁺ 422, found 422.

### Example 50

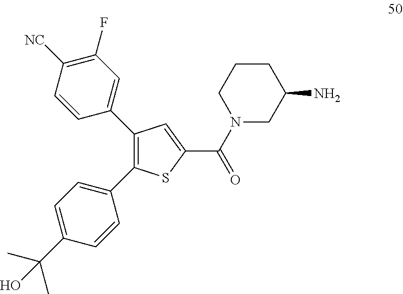 [SMILES: CC(C)(O)c1ccc(-c2sc(C(=O)N3CCC[C@@H](N)C3)cc2-c2ccc(C#N)c(F)c2)cc1]

Synthetic Route:

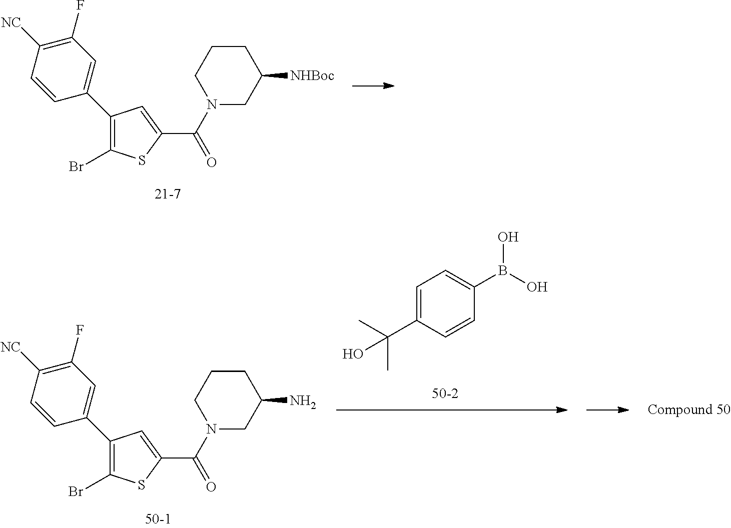 [SMILES: CC(C)(C)OC(=O)N[C@@H]1CCCN(C(=O)c2cc(-c3ccc(C#N)c(F)c3)c(Br)s2)C1 ; N#Cc1ccc(-c2cc(C(=O)N3CCC[C@@H](N)C3)sc2Br)cc1F ; CC(C)(O)c1ccc(B(O)O)cc1]

### Step 1

Compound 21-7 (500 mg, 983 μmol) was dissolved in ethyl acetate (30.0 mL), and hydrochloride ethyl acetate solution (4 mol/L, 2.46 mL) was added thereto, and the reaction mixture was stirred and reacted for 12 hours at 25° C. The reaction mixture was concentrated to obtain crude compound 50-1, which was directly used in the next reaction step. MS-ESI calculated for [M+H]⁺ 410, found 410.

### Step 2

Under nitrogen atmosphere, compound 50-1 (50.0 mg, 122 μmol), compound 50-2 (26.5 mg, 147 μmol), and sodium carbonate (26.0 mg, 245 μmol) were dissolved in dioxane (8.00 mL) and water (2.00 mL), and the reaction mixture was added with [1,1′-bis(diphenylphosphino)ferrocene]dichloropalladium(II) (8.96 mg, 12.3 μmol), and the reaction mixture was stirred and reacted for 12 hours at 100° C. The reaction mixture was concentrated under reduced pressure, and the residue was purified by preparative high performance liquid chromatography (chromatographic column: Phenomenex Genimi NX C18 150*40 mm*5 μm, mobile phase A: ammonia aqueous solution with a volume fraction of 0.05%; mobile phase B: 10 mM ammonium bicarbonate solution in acetonitrile; B %: 28% to 58%, 10 minutes) to obtain compound 50. ¹H NMR (400 MHz, CD₃OD) δ=7.67 (t, J=7.6 Hz, 1H), 7.58-7.48 (m, 3H), 7.36-7.22 (m, 4H), 4.59 (br, 1H), 4.40-4.32 (m, 1H), 4.28-4.20 (m, 1H), 3.09-2.82 (m, 2H), 2.14-2.00 (m, 1H), 1.94-1.79 (m, 1H), 1.69-1.57 (m, 1H), 1.54 (s, 6H), 1.50-1.40 (m, 1H). MS-ESI calculated for [M+H]⁺ 464, found 464.

### Example 51

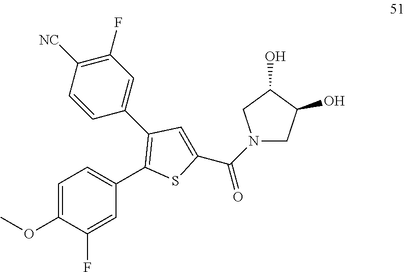 [SMILES: COc1ccc(-c2sc(C(=O)N3C[C@H](O)[C@@H](O)C3)cc2-c2ccc(C#N)c(F)c2)cc1F]

Synthetic Route:

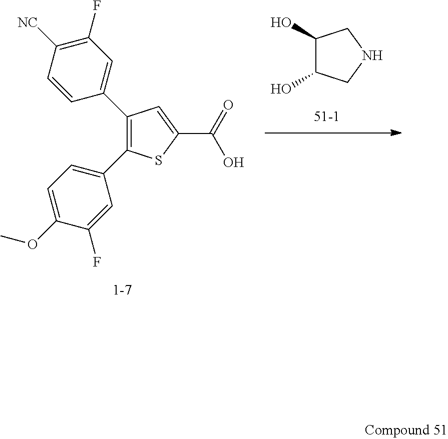 [SMILES: COc1ccc(-c2sc(C(=O)O)cc2-c2ccc(C#N)c(F)c2)cc1F ; O[C@H]1CNC[C@@H]1O]

Under nitrogen atmosphere, compound 1-7 (150 mg, 404 μmol), 2-(7-azabenzotriazol-1-yl)-N,N,N′,N′-tetramethyluronium hexafluorophosphate (230 mg, 606 μmol), and N,N-diisopropylethylamine (156 mg, 1.21 mmol) were dissolved in N,N-dimethylformamide (10.0 mL), and then compound 51-1 (50.0 mg, 485 μmol) was added thereto, and the reaction mixture was stirred and reacted for 12 hours at 25° C. The reaction mixture was added with water (50 mL), and extracted with ethyl acetate (50 mL×2). The organic phase was washed with saturated brine (50 mL×1), dried over anhydrous sodium sulfate, filtered, and concentrated under reduced pressure. The residue was purified by preparative high performance liquid chromatography (chromatographic column: Xtimate C18 150*40 mm*5 μm; mobile phase A: hydrochloric acid aqueous solution with a volume fraction of 0.05%; mobile phase B: acetonitrile; B %: 27% to 57%, 10 minutes) to obtain compound 51. ¹H NMR (400 MHz, CD₃OD) δ=7.72-7.61 (m, 2H), 7.30 (dd, J=1.2, 10.0 Hz, 1H), 7.25 (dd, J=1.2, 8.0 Hz, 1H), 7.13-7.00 (m, 3H), 4.27-4.20 (m, 1H), 4.19-4.10 (m, 2H), 3.89 (s, 3H), 3.86-3.79 (m, 2H), 3.65-3.55 (m, 1H). MS-ESI calculated for [M+H]⁺ 457, found 457.

### Example 52

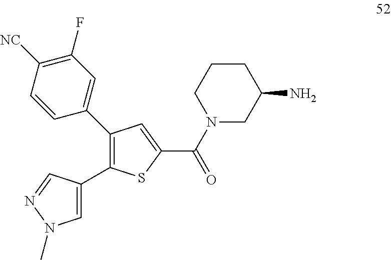 [SMILES: Cn1cc(-c2sc(C(=O)N3CCC[C@@H](N)C3)cc2-c2ccc(C#N)c(F)c2)cn1]

Synthetic Route:

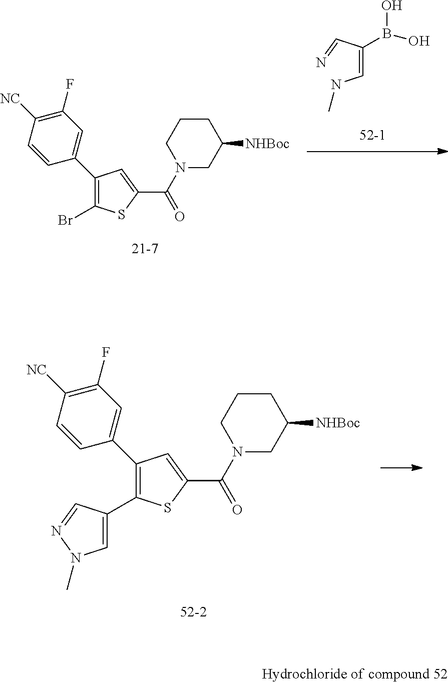

### Step 1

Under nitrogen atmosphere, compound 21-7 (168 mg, 331 μmol), compound 52-1 (50.0 mg, 397 μmol), and sodium carbonate (70.1 mg, 662 μmol) were dissolved in dioxane (32.0 mL) and water (8.00 mL), and the reaction mixture was added with [1,1′-bis(diphenylphosphino)ferrocene]dichloropalladium(II) (24.2 mg, 33.1 μmol), and the reaction mixture was stirred and reacted for 12 hours at 100° C. The reaction mixture was concentrated under reduced pressure, and the residue was purified by silica gel column chromatography (0/1, petroleum ether/ethyl acetate, R_{f}=0.28) to obtain compound 52-2. MS-ESI calculated for [M+Na]⁺ 532, found 532.

### Step 2

Compound 52-2 (110 mg, 162 μmol) was dissolved in ethyl acetate (20.0 mL), and hydrochloride ethyl acetate solution (4 mol/L, 401 μL) was added thereto, and the reaction mixture was stirred and reacted for 12 hours at 25° C. After the reaction mixture was concentrated under reduced pressure, the residue was purified by preparative high performance liquid chromatography (chromatographic column: Xtimate C18 150*40 mm*5 μm, mobile phase A: hydrochloric acid aqueous solution with a volume fraction of 0.05%; mobile phase B: acetonitrile; B %: 7% to 37%, 10 minutes) to obtain the hydrochloride of compound 52. ¹H NMR (400 MHz, CD₃OD) δ=7.76 (t, J=7.6 Hz, 1H), 7.69 (s, 1H), 7.49 (s, 1H), 7.45-7.34 (m, 3H), 4.37 (d, J=11.2 Hz, 1H), 4.14 (d, J=13.6 Hz, 1H), 3.88 (s, 3H), 3.58-3.37 (m, 3H), 2.27-2.14 (m, 1H), 1.92-1.87 (m, 1H), 1.81-1.64 (m, 2H). MS-ESI calculated for [M+H]⁺ 410, found 410.

### Example 53

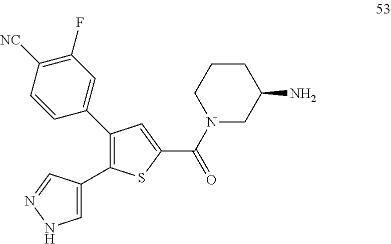 [SMILES: N#Cc1ccc(-c2cc(C(=O)N3CCC[C@@H](N)C3)sc2-c2cn[nH]c2)cc1F]

Synthetic Route:

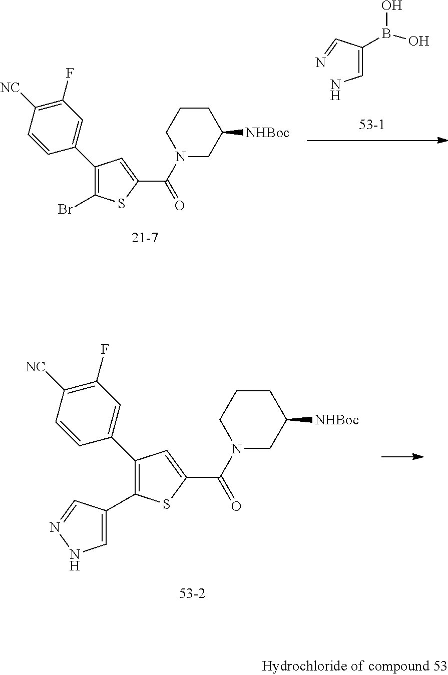 [SMILES: CC(C)(C)OC(=O)N[C@@H]1CCCN(C(=O)c2cc(-c3ccc(C#N)c(F)c3)c(Br)s2)C1 ; N=C/C(=C\N)B(O)O ; CC(C)(C)OC(=O)N[C@@H]1CCCN(C(=O)c2cc(-c3ccc(C#N)c(F)c3)c(-c3cn[nH]c3)s2)C1]

### Step 1

Under nitrogen atmosphere, compound 21-7 (300 mg, 590 μmol), compound 53-1 (79.2 mg, 708 μmol), and sodium carbonate (125 mg, 1.18 mmol) were dissolved in dioxane (32.0 mL) and water (4.00 mL), and the reaction mixture was added with [1,1′-bis(diphenylphosphino)ferrocene]dichloropalladium(II) (43.2 mg, 59.1 μmol), and the reaction mixture was stirred and reacted for 12 hours at 100° C. The reaction mixture was concentrated under reduced pressure, and the residue was purified by silica gel column chromatography (0/1, petroleum ether/ethyl acetate, R_{f}=0.18) to obtain compound 53-2. MS-ESI calculated for [M+Na]⁺ 518, found 518.

### Step 2

Compound 53-2 (70.0 mg, 141 μmol) was dissolved in ethyl acetate (15.0 mL), and hydrochloride ethyl acetate solution (4 mol/L, 353 μL) was added thereto, and the reaction mixture was stirred and reacted for 2 hours at 25° C. After the reaction mixture was concentrated under reduced pressure, the residue was purified by preparative high performance liquid chromatography (chromatographic column: Xtimate C18 150*40 mm*5 μm, mobile phase A: hydrochloric acid aqueous solution with a volume fraction of 0.05%; mobile phase B: acetonitrile; B %: 5% to 35%, 10 minutes) to obtain the hydrochloride of compound 53. ¹H NMR (400 MHz, CD₃OD) δ=7.76 (t, J=7.6 Hz, 1H), 7.64 (s, 2H), 7.51 (s, 1H), 7.45-7.34 (m, 2H), 4.38 (d, J=11.2 Hz, 1H), 4.23-4.07 (m, 1H), 3.59-3.36 (m, 3H), 2.26-2.14 (m, 1H), 1.96-1.85 (m, 1H), 1.81-1.63 (m, 2H). MS-ESI calculated for [M+H]⁺ 396, found 396.

### Example 54

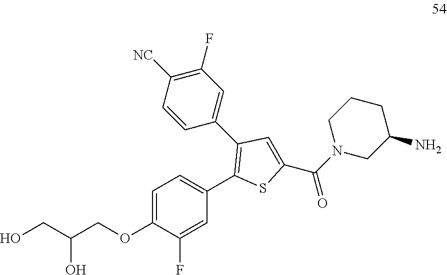 [SMILES: N#Cc1ccc(-c2cc(C(=O)N3CCC[C@@H](N)C3)sc2-c2ccc(OCC(O)CO)c(F)c2)cc1F]

Synthetic Route:

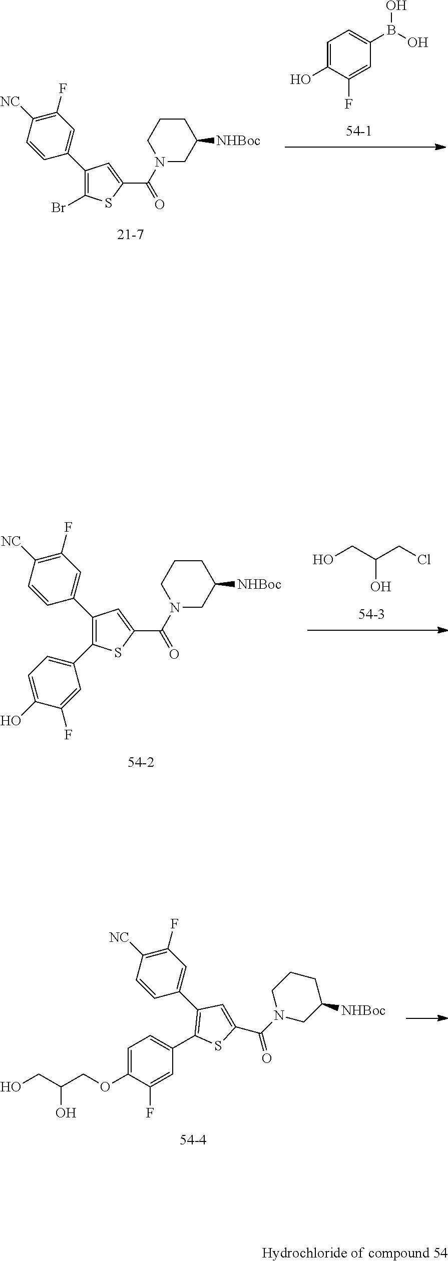 [SMILES: CC(C)(C)OC(=O)N[C@@H]1CCCN(C(=O)c2cc(-c3ccc(C#N)c(F)c3)c(Br)s2)C1 ; OB(O)c1ccc(O)c(F)c1 ; CC(C)(C)OC(=O)N[C@@H]1CCCN(C(=O)c2cc(-c3ccc(C#N)c(F)c3)c(-c3ccc(O)c(F)c3)s2)C1 ; OCC(O)CCl ; CC(C)(C)OC(=O)N[C@@H]1CCCN(C(=O)c2cc(-c3ccc(C#N)c(F)c3)c(-c3ccc(OCC(O)CO)c(F)c3)s2)C1]

### Step 1

Under nitrogen atmosphere, compound 21-7 (326 mg, 641 μmol), compound 54-1 (120 mg, 770 μmol), and sodium carbonate (136 mg, 1.28 mmol) were dissolved in dioxane (32.00 mL) and water (8.00 mL), and the reaction mixture was added with [1,1′-bis(diphenylphosphino)ferrocene]dichloropalladium(II) (46.9 mg, 64.1 μmol), and the reaction mixture was stirred and reacted for 12 hours at 100° C. The reaction mixture was concentrated under reduced pressure, and the residue was purified by silica gel column chromatography (1/1, petroleum ether/ethyl acetate, R_{f}=0.25) to obtain compound 54-2. MS-ESI calculated for [M+Na]⁺ 562, found 562.

### Step 2

Compound 54-2 (50.0 mg, 92.6 μmol) was dissolved in ethanol (10.0 mL), and then 1.00 mL of sodium hydroxide (33.8 mg, 844 μmol) aqueous solution was added thereto. The reaction mixture was stirred and reacted for 2 hours at 78° C. and then cooled to 50° C. 10.0 mL of a solution of compound 54-3 (51.2 mg, 463 μmol) in ethanol was added slowly in batches. The reaction mixture was stirred and reacted for another 15 hours at 78° C. The reaction mixture was added with water (50 mL) and extracted with ethyl acetate (50 mL×2). The organic phase was washed with saturated brine (50 mL×1), dried over anhydrous sodium sulfate, filtered, and concentrated under reduced pressure to obtain crude compound 54-4, which was used directly in the next reaction step. MS-ESI calculated for [M+H]⁺ 558, found 558.

### Step 3

Compound 54-4 (65.0 mg, 106 μmol) was dissolved in dichloromethane (10.0 mL), and trifluoroacetic acid (78.4 μL, 1.06 mmol) was added thereto, and the reaction mixture was stirred and reacted for 1 hour at 25° C. After the reaction mixture was concentrated under reduced pressure, the residue was purified by preparative high performance liquid chromatography (chromatographic column: Xtimate C18 150*40 mm*5 μm, mobile phase A: hydrochloric acid aqueous solution with a volume fraction of 0.05%; mobile phase B: acetonitrile; B %: 10% to 50%, 10 minutes) to obtain the hydrochloride of compound 54. ¹H NMR (400 MHz, CD₃OD) δ=7.69 (t, J=7.6 Hz, 1H), 7.56 (s, 1H), 7.33 (d, J=10.4 Hz, 1H), 7.26 (d, J=8.0 Hz, 1H), 7.17-7.10 (t, J=8.8 Hz, 1H), 7.09-7.00 (m, 2H), 4.39 (d, J=12.0 Hz, 1H), 4.17-4.10 (m, 2H), 4.09-4.03 (m, 1H), 4.02-3.97 (m, 1H), 3.72-3.63 (m, 2H), 3.57-3.37 (m, 3H), 2.26-2.16 (m, 1H), 1.98-1.86 (m, 1H), 1.84-1.66 (m, 2H). MS-ESI calculated for [M+H]⁺ 514, found 514.

### Example 55

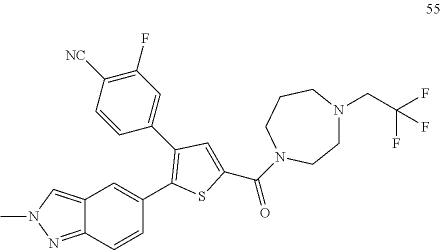 [SMILES: Cn1cc2cc(-c3sc(C(=O)N4CCCN(CC(F)(F)F)CC4)cc3-c3ccc(C#N)c(F)c3)ccc2n1]

Synthetic Route:

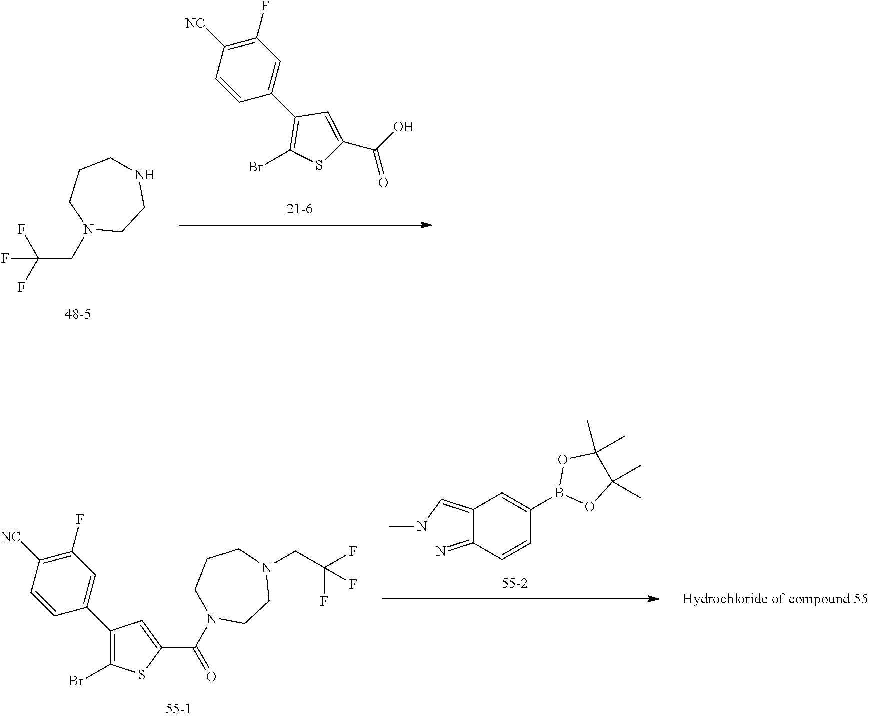

### Step 1

Under nitrogen atmosphere, compound 21-6 (130 mg, 0.399 mmol) was dissolved in anhydrous N,N-dimethylformamide (2.00 mL), and then triethylamine (121 mg, 1.20 mmol), compound 48-5 (105 mg, 0.478 mmol), and 2-(7-azabenzotriazol-1-yl)-N,N,N′,N′-tetramethyluronium hexafluorophosphate (227 mg, 0.598 mmol) were added thereto. The reaction mixture was stirred for 12 hours at 25° C. The reaction mixture was added with water (20 mL), and extracted with ethyl acetate (20 mL×1). The organic phase was washed with saturated brine (20 mL×1), dried over anhydrous sodium sulfate, filtered, and concentrated under reduced pressure. The residue was purified by silica gel column chromatography (1/1, petroleum ether/ethyl acetate, R_{f}=0.20) to obtain compound 55-1. MS-ESI calculated for [M+H]⁺ 491, found 491.

### Step 2

Under nitrogen atmosphere, compound 55-1 (185 mg, 0.377 mmol), compound 55-2 (97.4 mg, 0.377 mmol), and potassium phosphate (240 mg, 1.13 mmol) were dissolved in dioxane (10.0 mL) and water (2.00 mL), and the reaction mixture was added with [1,1′-bis(diphenylphosphino)ferrocene]dichloropalladium(II) (27.6 mg, 0.0377 mmol), and the reaction mixture was stirred and reacted for 12 hours at 100° C. The reaction mixture was added with water (20 mL), and extracted with ethyl acetate (20 mL×1). The organic phase was washed with saturated brine (20 mL×3), dried over anhydrous sodium sulfate, filtered, and concentrated under reduced pressure. The residue was purified by silica gel column chromatography (1/1, petroleum ether/ethyl acetate, R_{f}=0.17), then subjected to preparative high performance liquid chromatography (chromatographic column: Xtimate C18 150*40 mm*5 μm; mobile phase: mobile phase A: hydrochloric acid aqueous solution with a volume fraction of 0.05%; mobile phase B: acetonitrile; B %: 40% to 70%, 10 minutes) to obtain the hydrochloride of compound 55. ¹H NMR (400 MHz, CD₃OD) δ 8.33 (s, 1H), 7.80 (s, 1H), 7.68-7.57 (m, 3H), 7.35-7.29 (m, 1H), 7.28-7.24 (m, 1H), 7.21-7.15 (m, 1H), 4.25 (s, 3H), 4.22-3.85 (br s, 6H), 3.64-3.38 (m, 4H), 2.40-2.17 (br s, 2H). MS-ESI calculated for [M+H]⁺ 542, found 542.

### Example 56

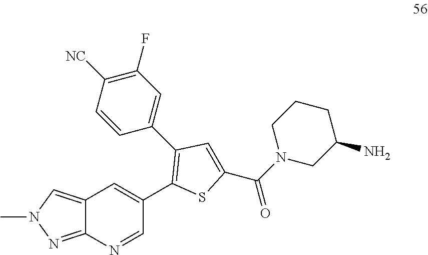 [SMILES: Cn1cc2cc(-c3sc(C(=O)N4CCC[C@@H](N)C4)cc3-c3ccc(C#N)c(F)c3)cnc2n1]

Synthetic Route:

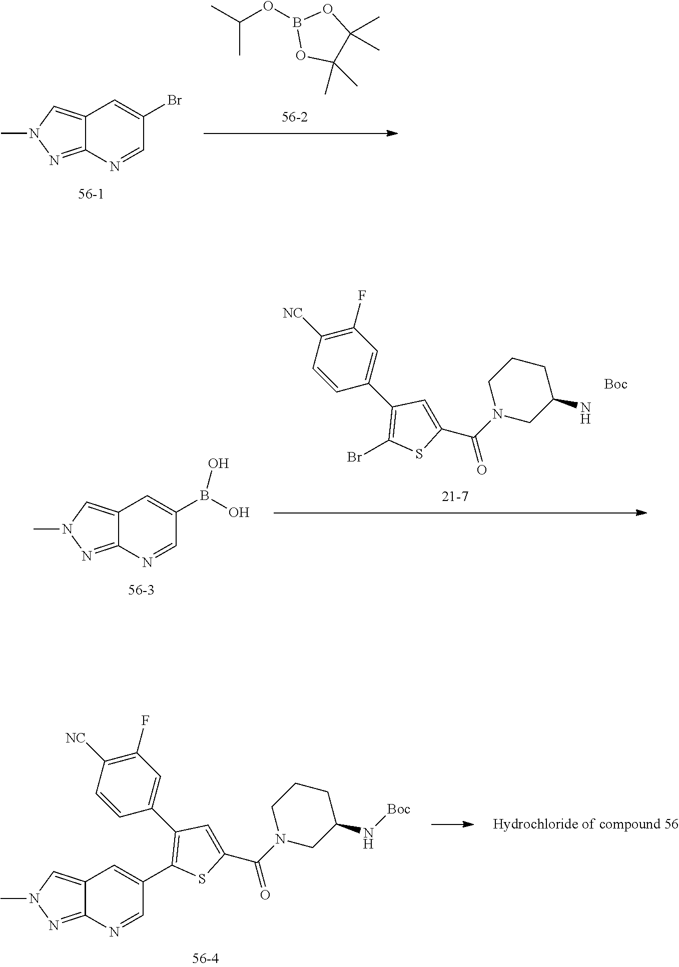

### Step 1

Under nitrogen atmosphere, compound 56-1 (100 mg, 0.472 mmol), compound 56-2 (132 mg, 0.707 mmol) were dissolved in anhydrous tetrahydrofuran (5.00 mL), then n-butyllithium (2.5 mol/L n-hexane solution, 377 μL) was added dropwise thereto at −78° C. The reaction mixture was stirred and reacted for 12 hours at 25° C., added with water (20 mL), and extracted with ethyl acetate (20 mL×1). The aqueous phase was concentrated under reduced pressure to obtain compound 56-3. MS-ESI calculated for [M+H]⁺ 178, found 178.

### Step 2

Under nitrogen atmosphere, compound 21-7 (110 mg, 0.216 mmol), compound 56-3 (57.4 mg, 0.325 mmol), and potassium phosphate (138 mg, 0.649 mmol) were dissolved in dioxane (10.0 mL) and water (2.00 mL), and the reaction mixture was added with [1,1′-bis(diphenylphosphino)ferrocene]dichloropalladium(II) (15.8 mg, 0.216 mmol). The reaction mixture was stirred and reacted for 12 hours at 100° C., added with water (20 mL), and extracted with ethyl acetate (20 mL×1). The organic phase was washed with saturated brine (20 mL×1), dried over anhydrous sodium sulfate, filtered, and concentrated under reduced pressure. The residue was purified by silica gel column chromatography (1/2, petroleum ether/ethyl acetate, R_{f}=0.19) to obtain compound 56-4. MS-ESI calculated for [M+H]⁺ 561, found 561.

### Step 3

Compound 56-4 (115 mg, 0.205 mmol) was dissolved in ethyl acetate (5.00 mL), and hydrochloride ethyl acetate solution (4 mol/L, 5 mL) was added dropwise thereto at 25° C. The reaction mixture was stirred for 12 hours at 25° C. The reaction mixture was concentrated under reduced pressure, and the residue was separated by preparative high performance liquid chromatography (chromatographic column: Xtimate C18 150*40 mm*5 μm; mobile phase: mobile phase A: hydrochloric acid aqueous solution with a volume fraction of 0.05%; mobile phase B: acetonitrile; B %: 3% to 33%, 10 minutes) to obtain the hydrochloride of compound 56. ¹H NMR (400 MHz, CD₃OD) δ 8.93-8.80 (m, 3H), 7.74-7.66 (m, 2H), 7.49 (d, J=10.0 Hz, 1H), 7.32 (d, J=8.0 Hz, 1H), 4.46-4.43 (m, 1H), 4.39 (s, 3H), 4.24-4.11 (m, 1H), 3.58-3.41 (m, 3H), 2.27-2.17 (m, 1H), 1.98-1.88 (m, 1H), 1.86-1.69 (m, 2H). MS-ESI calculated for [M+H]⁺ 461, found 461.

### Example 57

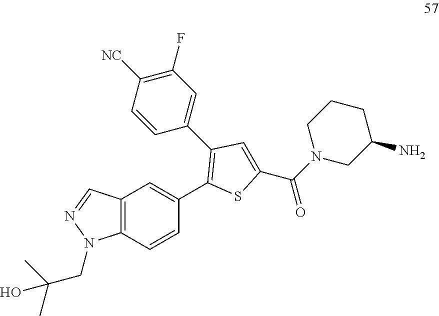 [SMILES: CC(C)(O)Cn1ncc2cc(-c3sc(C(=O)N4CCC[C@@H](N)C4)cc3-c3ccc(C#N)c(F)c3)ccc21]

Synthetic Route:

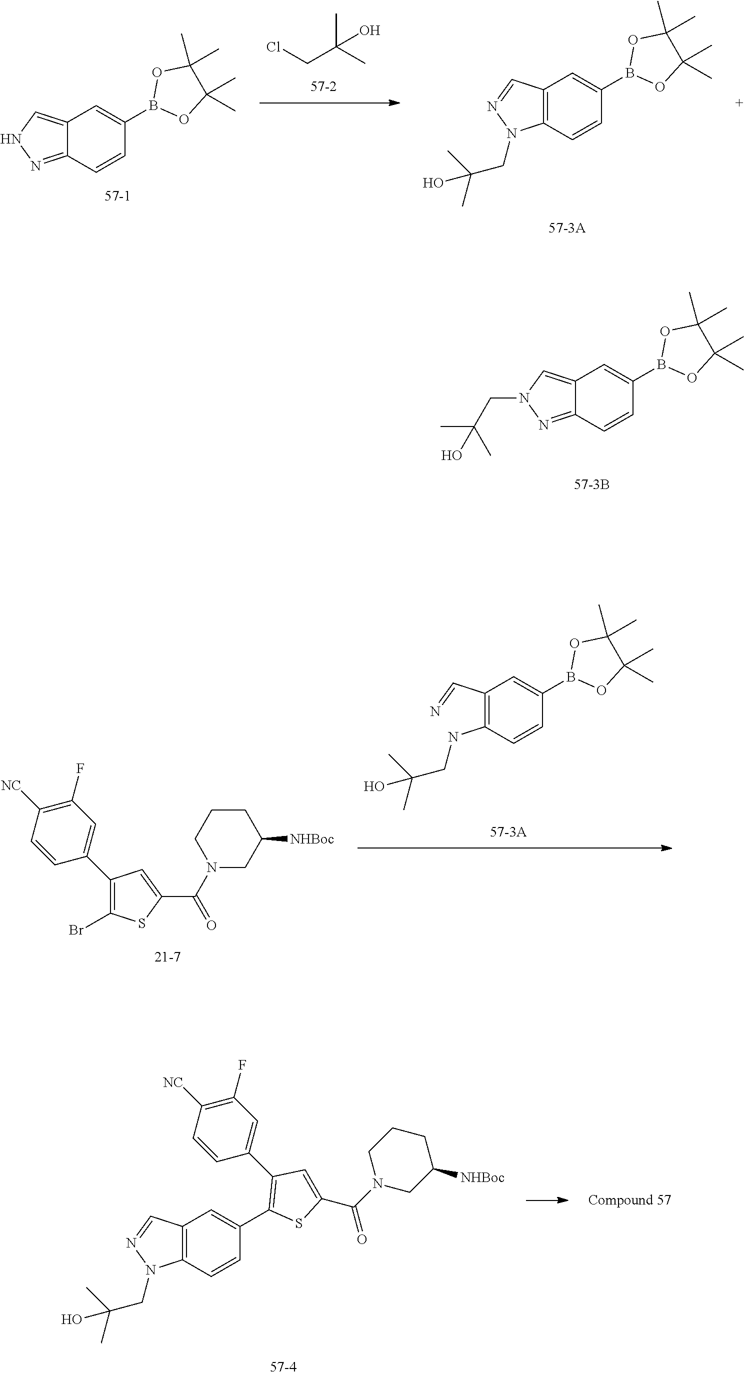

### Step 1

Compounds 57-1 (100 mg, 410 μmol) and 57-2 (210 μL, 2.05 mmol) were dissolved in N,N-dimethylformamide (2.00 mL), and potassium carbonate (283 mg, 2.05 mmol) was added thereto, and the reaction mixture was stirred and reacted for 48 hours at 100° C. The reaction mixture was added with water (50 mL), and extracted with ethyl acetate (50 mL×2). The organic phase was washed with saturated brine (50 mL×1), dried over anhydrous sodium sulfate, filtered, and concentrated under reduced pressure. The residue was purified by silica gel column chromatography (1/1, petroleum ether/ethyl acetate, Rf_{P1}=0.67, Rf_{P2}=0.47) to obtain compounds 57-3A and 57-3B. Compound 57-3A MS-ESI calculated for [M+H]⁺ 317, found 317; compound 57-3B MS-ESI calculated for [M+H]⁺ 317, found 317.

### Step 2

Under nitrogen atmosphere, compound 21-7 (68.3 mg, 134 μmol), compound 57-3A (104 mg, 161 μmol), and sodium carbonate (28.5 mg, 269 μmol) were dissolved in dioxane (8.00 mL) and water (2.00 mL), and the reaction mixture was added with [1,1′-bis(diphenylphosphino)ferrocene]dichloropalladium(II) (9.83 mg, 13.4 μmol), and the reaction mixture was stirred and reacted for 12 hours at 100° C. The reaction mixture was concentrated under reduced pressure, and the residue was purified by silica gel column chromatography (1/2, petroleum ether/ethyl acetate, R_{f}=0.19) to obtain compound 57-4. ¹H NMR (400 MHz, CD₃OD) δ=8.05 (s, 1H), 7.78 (s, 1H), 7.62 (d, J=8.8 Hz, 1H), 7.58-7.48 (m, 2H), 7.30-7.19 (m, 2H), 6.81-6.78 (m, 1H), 4.53-4.42 (m, 1H), 4.37 (s, 2H), 4.30-4.20 (m, 1H), 3.69-3.56 (m, 1H), 3.19-3.05 (m, 2H), 2.10-2.00 (m, 1H), 1.94-1.83 (m, 1H), 1.72-1.53 (m, 2H), 1.43 (s, 9H), 1.24 (s, 6H). MS-ESI calculated for [M+H]⁺ 618, found 618.

### Step 3

Compound 57-4 (37.0 mg, 59.9 μmol) was dissolved in dichloromethane (10.0 mL), and the reaction mixture was added with trimethylsilyl trifluoromethanesulfonate (21.6 μL, 120 μmol) at 0° C., stirred, and reacted for 1 hour at 25° C. The reaction mixture was added with saturated sodium bicarbonate aqueous solution (20 mL), and extracted with dichloromethane (20 mL×3). The organic phase was washed with saturated brine (20.0 mL×1), dried over anhydrous sodium sulfate, filtered, and concentrated under reduced pressure. The residue was purified by preparative high performance liquid chromatography (chromatographic column: Welch Xtimate C18 150*25 mm*5 μm, mobile phase A: 10 mM ammonium bicarbonate aqueous solution; mobile phase B: acetonitrile; B %: 35% to 65%, 9 minutes) to obtain compound 57. ¹H NMR (400 MHz, CD₃OD) δ=8.06 (s, 1H), 7.79 (s, 1H), 7.67-7.59 (m, 2H), 7.55 (s, 1H), 7.32 (d, J=10.4 Hz, 1H), 7.28-7.21 (m, 2H), 4.41-4.32 (m, 3H), 4.28-4.20 (m, 1H), 3.24-3.08 (m, 1H), 3.07-3.00 (m, 1H), 2.93-2.83 (m, 1H), 2.13-1.99 (m, 1H), 1.92-1.79 (m, 1H), 1.73-1.55 (m, 1H), 1.51-1.37 (m, 1H), 1.24 (s, 6H). MS-ESI calculated for [M+H]⁺ 518, found 518.

### Example 58

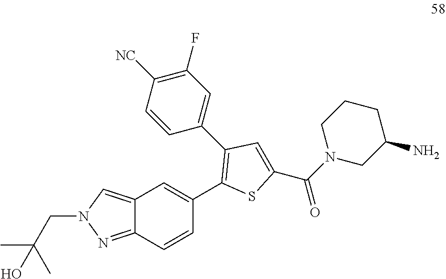 [SMILES: CC(C)(O)Cn1cc2cc(-c3sc(C(=O)N4CCC[C@@H](N)C4)cc3-c3ccc(C#N)c(F)c3)ccc2n1]

Synthetic Route:

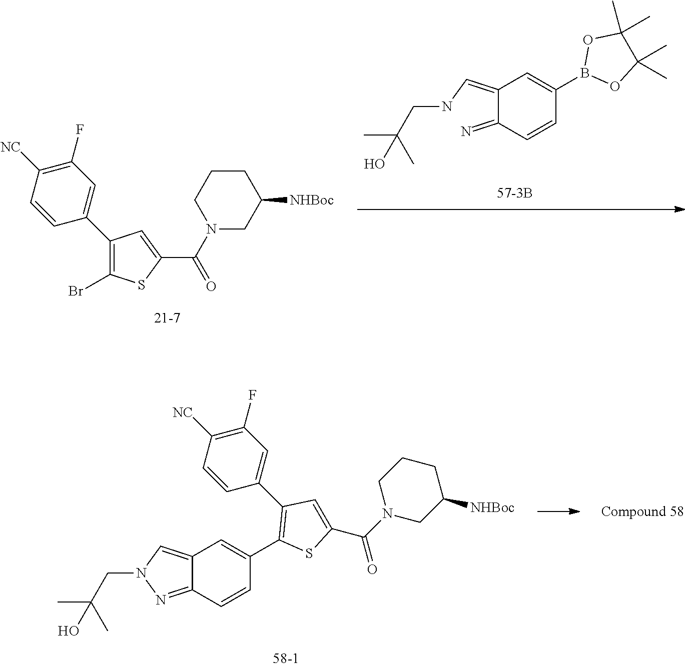 [SMILES: CC(C)(C)OC(=O)N[C@@H]1CCCN(C(=O)c2cc(-c3ccc(C#N)c(F)c3)c(Br)s2)C1 ; CC(C)(O)CN/C=C1/C=C(B2OC(C)(C)C(C)(C)O2)C=CC1=N ; CC(C)(O)Cn1cc2cc(-c3sc(C(=O)N4CCC[C@@H](NC(=O)OC(C)(C)C)C4)cc3-c3ccc(C#N)c(F)c3)ccc2n1]

### Step 1

Under nitrogen atmosphere, compound 21-7 (16.9 mg, 33 μmol), compound 57-3B (15.0 mg, 39.9 μmol), and sodium carbonate (7.04 mg, 66.4 μmol) were dissolved in dioxane (4.00 mL) and water (1.00 mL), and the reaction mixture was added with [1,1′-bis(diphenylphosphino)ferrocene]dichloropalladium(II) (2.43 mg, 3.32 μmol), and the reaction mixture was stirred and reacted for 12 hours at 100° C. The reaction mixture was concentrated under reduced pressure, and the residue was purified by silica gel column chromatography (1/2, petroleum ether/ethyl acetate, R_{f}=0.14) to obtain compound 58-1. ¹H NMR (400 MHz, CD₃OD) δ=8.30 (s, 1H), 7.81 (s, 1H), 7.71-7.48 (m, 3H), 7.31 (d, J=8.0 Hz, 1H), 7.13 (d, J=8.8 Hz, 1H), 6.81 (d, J=6.4 Hz, 1H), 4.54-4.44 (m, 1H), 4.42 (s, 2H), 4.35-4.20 (m, 1H), 3.74-3.58 (m, 1H), 3.21-3.04 (m, 2H), 2.12-1.98 (m, 1H), 1.94-1.84 (m, 1H), 1.73-1.55 (m, 2H), 1.45 (s, 9H), 1.24 (s, 6H). MS-ESI calculated for [M+H]⁺ 618, found 618.

### Step 3

Compound 58-1 (18.0 mg, 29.1 μmol) was dissolved in dichloromethane (10.0 mL), and the reaction mixture was added with trimethylsilyl trifluoromethanesulfonate (10.5 μL, 58.3 μmol) at 0° C., stirred, and reacted for 1 hour at 25° C. The reaction mixture was added with saturated sodium bicarbonate aqueous solution (20 mL), and extracted with dichloromethane (20 mL×3). The organic phase was washed with saturated brine (20.0 mL×1), dried over anhydrous sodium sulfate, filtered, and concentrated under reduced pressure. The residue was purified by preparative high performance liquid chromatography (chromatographic column: Welch Xtimate C18 150*25 mm*5 μm, mobile phase A: 10 mM ammonium bicarbonate aqueous solution; mobile phase B: acetonitrile; B %: 36% to 66%, 9 minutes) to obtain compound 58. ¹H NMR (400 MHz, CD₃OD) δ=8.31 (s, 1H), 7.82 (s, 1H), 7.70-7.59 (m, 2H), 7.58 (s, 1H), 7.37 (d, J=10.0 Hz, 1H), 7.30 (d, J=8.0 Hz, 1H), 7.15 (d, J=8.8 Hz, 1H), 4.44-4.35 (m, 3H), 4.29-4.21 (m, 1H), 3.26-3.14 (m, 1H), 3.09-2.98 (m, 1H), 2.93-2.85 (m, 1H), 2.11-2.06 (m, 1H), 1.95-1.84 (m, 1H), 1.69-1.58 (m, 1H), 1.54-1.42 (m, 1H), 1.24 (s, 6H). MS-ESI calculated for [M+H]⁺ 518, found 518.

### Example 59

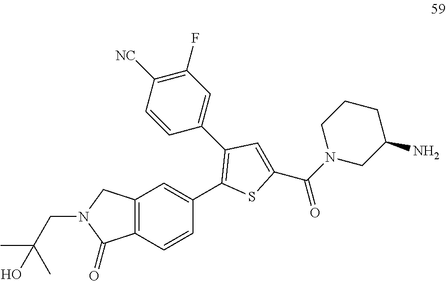 [SMILES: CC(C)(O)CN1Cc2cc(-c3sc(C(=O)N4CCC[C@@H](N)C4)cc3-c3ccc(C#N)c(F)c3)ccc2C1=O]

Synthetic Route:

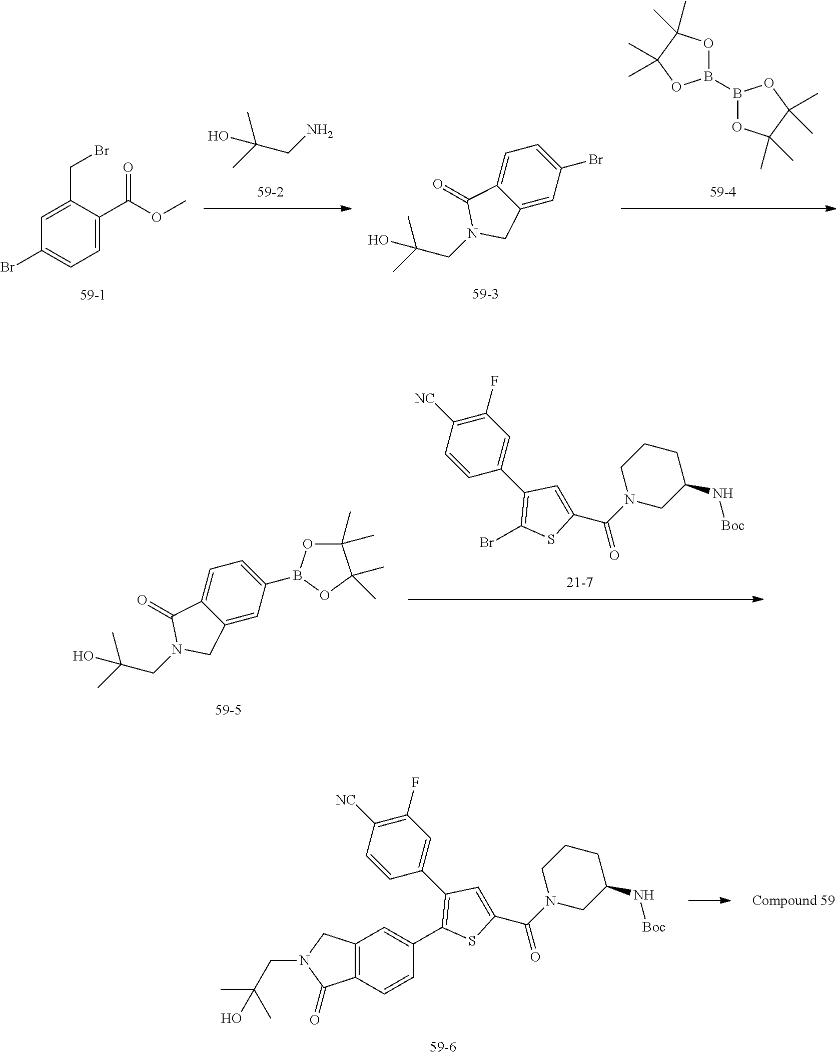

### Step 1

Under nitrogen atmosphere, compound 59-1 (810 mg, 2.63 mmol) was dissolved in anhydrous toluene (20.0 mL), and then triethylamine (840 mg, 8.30 mmol) and 1-amino-2-methylpropan-2-ol (16-2) (281 mg, 3.16 mmol) were added thereto. The reaction mixture was stirred for 4 hours at 110° C., and then concentrated under reduced pressure. The residue was purified by silica gel column chromatography (10/1, dichloromethane/methanol, R_{f}=0.18) to obtain compound 59-3. ¹H NMR (400 MHz, CDCl₃) δ 7.74-7.70 (m, 1H), 7.64-7.59 (m, 2H), 4.59 (s, 2H), 3.60 (s, 2H), 1.29 (s, 6H).

### Step 2

Under nitrogen atmosphere, compound 59-3 (720 mg, 2.53 mmol), bis(pinacolato)diboron (59-4) (772 mg, 3.04 mmol), and potassium acetate (746 mg, 7.60 mmol) were dissolved in dioxane (10.0 mL), and the reaction mixture was added with [1,1′-bis(diphenylphosphino)ferrocene]dichloropalladium(II) (185 mg, 0.253 mmol), and the reaction mixture was stirred and reacted for 2 hours at 95° C., and concentrated under reduced pressure. The residue was purified by silica gel column chromatography (0/1, petroleum ether/ethyl acetate, R_{f}=0.26) to obtain compound 59-5. ¹H NMR (400 MHz, CDCl₃) δ 7.94-7.84 (m, 3H), 4.59 (s, 2H), 3.63 (s, 2H), 1.37 (s, 9H), 1.30 (s, 6H).

### Step 3

Under nitrogen atmosphere, compound 21-7 (150 mg, 0.295 mmol), compound 59-5 (107 mg, 0.325 mmol), and sodium carbonate (93.8 mg, 0.885 mmol) were dissolved in dioxane (10.0 mL) and water (2.00 mL), and the reaction mixture was added with [1,1′-bis(diphenylphosphino)ferrocene]dichloropalladium(II) (21.6 mg, 0.295 mmol). The reaction mixture was stirred and reacted for 2 hours at 80° C., added with water (20 mL), and extracted with ethyl acetate (20 mL×1). The organic phase was washed with saturated brine (20 mL×1), dried over anhydrous sodium sulfate, filtered, and concentrated under reduced pressure. The residue was purified by silica gel column chromatography (1/2, petroleum ether/ethyl acetate, R_{f}=0.19) to obtain compound 59-6. MS-ESI calculated for [M+H]⁺ 633, found 633.

### Step 4

Under nitrogen atmosphere, compound 59-6 (160 mg, 0.253 mmol) was dissolved in anhydrous dichloromethane (5 mL), and trimethylsilyl trifluoromethanesulfonate (112 mg, 0.506 mmol) was added dropwise at 0° C. The reaction mixture was stirred for 1 hour at 25° C., added with saturated sodium bicarbonate aqueous solution (20 mL), and extracted with dichloromethane (20 mL×1). The organic phase was dried over anhydrous sodium sulfate, filtered, concentrated under reduced pressure, and separated by preparative high performance liquid chromatography (chromatographic column: Phenomenex Genimi NX C18 150*40 mm*5 μm; mobile phase: mobile phase A: ammonia aqueous solution with a volume fraction of 0.04% and 10 mmol/L sodium bicarbonate aqueous solution; mobile phase B: acetonitrile; B %: 20 to 50%, 10 minutes) to obtain compound 59. ¹H NMR (400 MHz, CD₃OD) δ 7.74 (d, J=8.0 Hz, 1H), 7.68-7.63 (m, 1H), 7.57-7.52 (m, 2H), 7.43-7.32 (m, 2H), 7.25-7.19 (m, 1H), 4.69 (s, 2H), 4.40-4.33 (m, 1H), 4.29-4.22 (m, 1H), 3.58 (s, 2H), 3.30-3.09 (m, 1H), 3.07-2.92 (m, 1H), 2.91-2.80 (m, 1H), 2.10-2.02 (m, 1H), 1.90-1.80 (m, 1H), 1.69-1.55 (m, 1H), 1.51-1.37 (m, 1H), 1.23 (s, 6H). MS-ESI calculated for [M+H]⁺ 533, found 533.

### Example 60

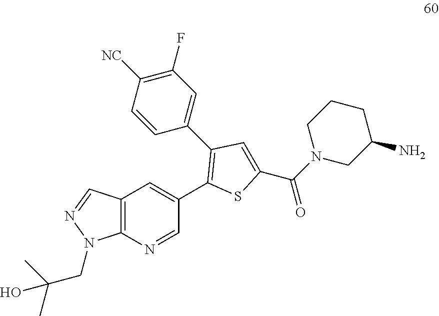 [SMILES: CC(C)(O)Cn1ncc2cc(-c3sc(C(=O)N4CCC[C@@H](N)C4)cc3-c3ccc(C#N)c(F)c3)cnc21]

Synthetic Route:

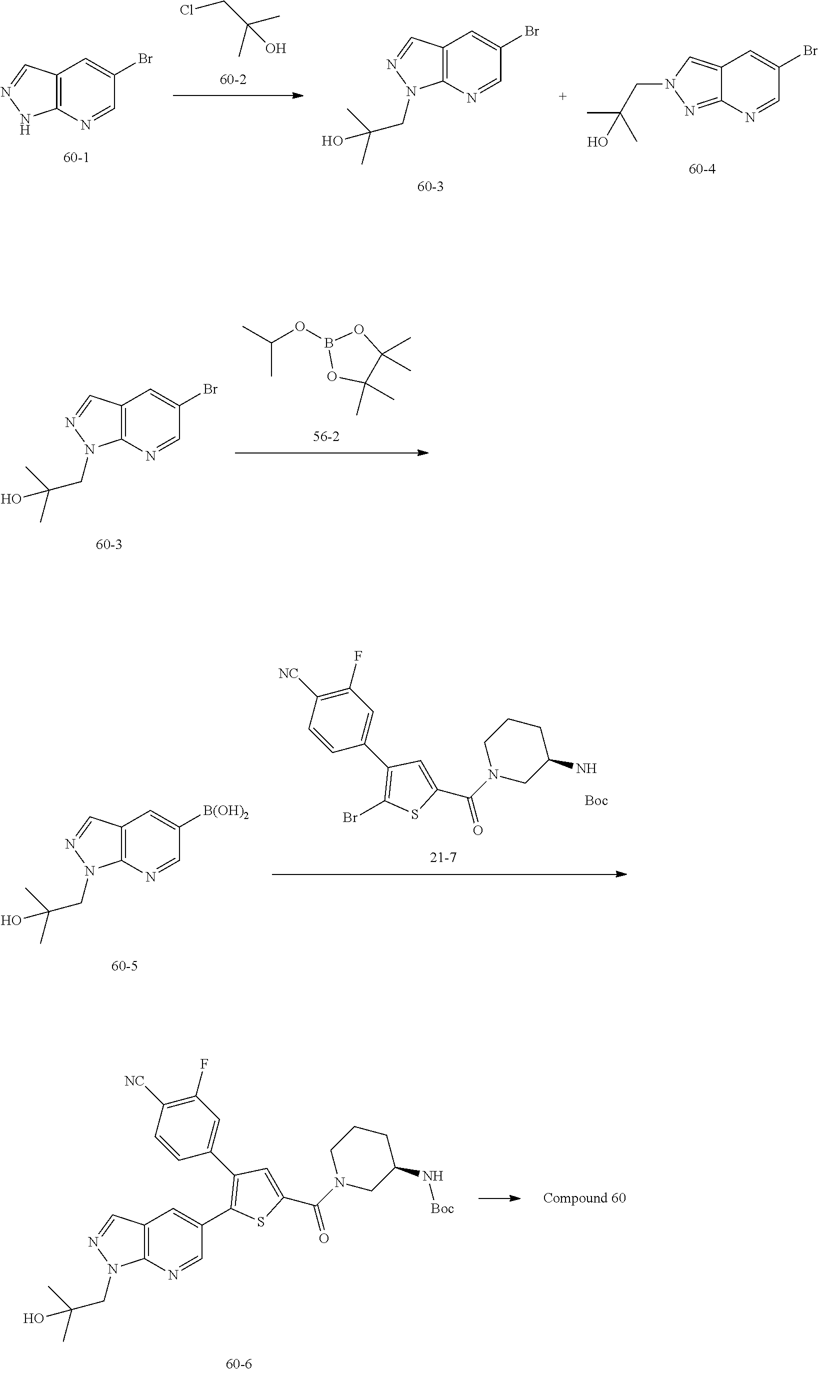

### Step 1

Under nitrogen atmosphere, compound 60-1 (2.10 g, 10.6 mmol) was dissolved in anhydrous N,N-dimethylformamide (20.0 mL), and then potassium carbonate (4.40 g, 31.8 mmol) and 1-amino-2-methylpropan-2-ol (2.30 g, 21.2 mmol) were added thereto, and the reaction mixture was stirred for 12 hours at 110° C., added with water (40 mL), and extracted with ethyl acetate (40 mL×1). The organic phase was washed with saturated brine (40 mL×1), dried over anhydrous sodium sulfate, filtered, and concentrated under reduced pressure. The residue was purified by silica gel column chromatography (1/2, petroleum ether/ethyl acetate, R_{f}=0.66, 0.33) to obtain compound 60-3 and compound 60-4. Compound 60-3 ¹H NMR (400 MHz, CDCl₃) δ 8.52 (d, J=2.0 Hz, 1H), 8.20 (d, J=2.0 Hz, 1H), 7.98 (s, 1H), 4.50 (s, 2H), 4.00 (s, 1H), 1.18 (s, 6H). MS-ESI calculated for [M+H]⁺ 271, found 271. Compound 60-4 ¹H NMR (400 MHz, CDCl₃) δ 8.67 (d, J=2.0 Hz, 1H), 8.20 (d, J=2.4 Hz, 1H), 7.98 (s, 1H), 4.39 (s, 2H), 3.98 (s, 1H), 1.22 (s, 6H). MS-ESI calculated for [M+H]⁺ 271, found 271.

### Step 2

Under nitrogen atmosphere, compound 60-3 (100 mg, 0.370 mmol), 2-isopropoxy-4,4,5,5-tetramethyl-1,3,2-dioxaborolane (56-2) (103 mg, 0.555 mmol) were dissolved in anhydrous tetrahydrofuran (5.00 mL), and n-butyllithium (2.5 mol/L n-hexane solution, 592 μL) was added dropwise thereto at −78° C. The reaction mixture was stirred and reacted for 12 hours at 25° C., added with water (20 mL), and extracted with ethyl acetate (20 mL×1). The aqueous phase was concentrated under reduced pressure to obtain compound 60-5. MS-ESI calculated for [M+H]⁺ 236, found 236.

### Step 3

Under nitrogen atmosphere, compound 21-7 (100 mg, 0.197 mmol), compound 60-5 (64.7 mg, 0.275 mmol), and sodium carbonate (62.5 mg, 0.590 mmol) were dissolved in dioxane (10.0 mL) and water (2.00 mL), and the reaction mixture was added with [1,1′-bis(diphenylphosphino)ferrocene]dichloropalladium(II) (14.4 mg, 0.197 mmol). The reaction mixture was stirred and reacted for 2 hours at 80° C., added with water (20 mL), and extracted with ethyl acetate (20 mL×1). The organic phase was washed with saturated brine (20 mL×1), dried over anhydrous sodium sulfate, filtered, and concentrated under reduced pressure. The residue was purified by silica gel column chromatography (1/2, petroleum ether/ethyl acetate, R_{f}=0.19) to obtain compound 60-6. MS-ESI calculated for [M+H]⁺ 619, found 619.

### Step 4

Under nitrogen atmosphere, compound 60-6 (60.0 mg, 0.097 mmol) was dissolved in anhydrous dichloromethane (5.00 mL), and trimethylsilyl trifluoromethanesulfonate (43.1 mg, 0.194 mmol) was added dropwise at 0° C. The reaction mixture was stirred for 1 hour at 25° C., added with saturated sodium bicarbonate aqueous solution (20 mL), and extracted with dichloromethane (20 mL×1). The organic phase was dried over anhydrous sodium sulfate, filtered, concentrated under reduced pressure, and separated by preparative high performance liquid chromatography (chromatographic column: Phenomenex Genimi NX C18 150*40 mm*5 μm; mobile phase: mobile phase A: ammonia aqueous solution with a volume fraction of 0.04% and 10 mmol/L sodium bicarbonate aqueous solution; mobile phase B: acetonitrile; B %: 20 to 50%, 10 minutes) to obtain compound 60. ¹H NMR (400 MHz, CD₃OD) δ 8.38 (d, J=2.0 Hz, 1H), 8.24 (d, J=2.0 Hz, 1H), 8.14 (s, 1H), 7.68-7.63 (m, 1H), 7.59 (s, 1H), 7.36 (d, J=10.0 Hz, 1H), 7.26-7.21 (m, 1H), 4.50 (s, 2H), 4.43-4.32 (m, 1H), 4.30-4.21 (m, 1H), 3.29-3.10 (m, 1H), 3.08-2.92 (m, 1H), 2.91-2.82 (m, 1H), 2.10-2.02 (m, 1H), 1.92-1.82 (m, 1H), 1.69-1.56 (m, 1H), 1.51-1.38 (m, 1H), 1.23 (s, 6H). MS-ESI calculated for [M+H]⁺ 519, found 519.

### Example 61

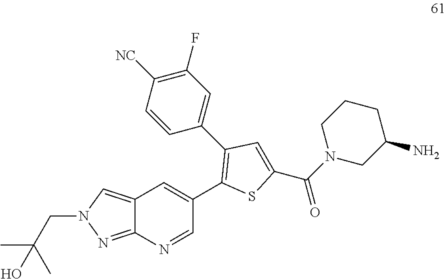 [SMILES: CC(C)(O)Cn1cc2cc(-c3sc(C(=O)N4CCC[C@@H](N)C4)cc3-c3ccc(C#N)c(F)c3)cnc2n1]

Synthetic Route:

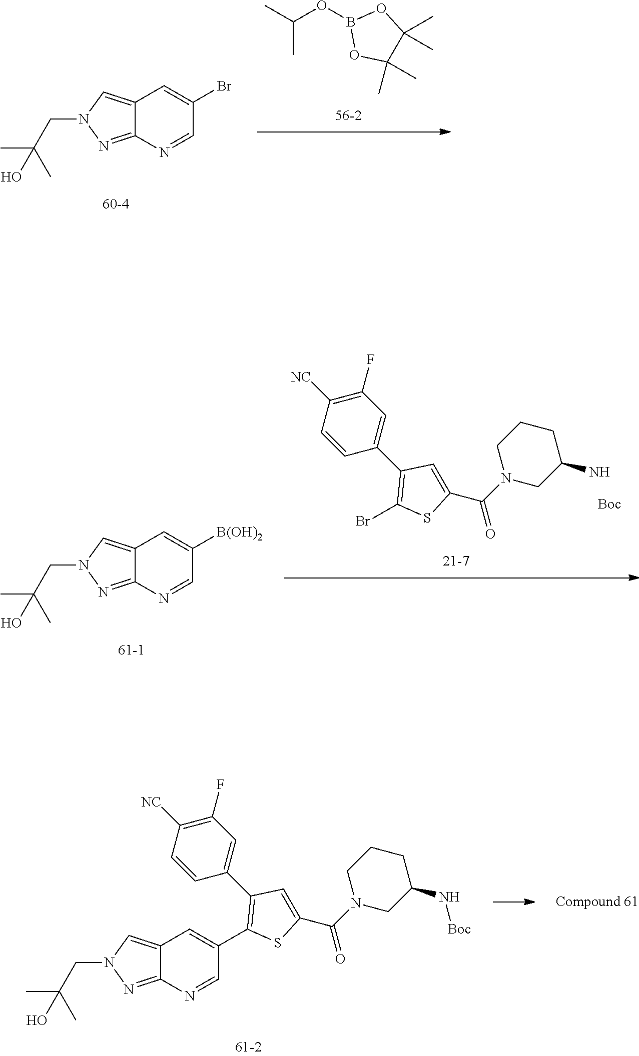 [SMILES: CC(C)(O)Cn1cc2cc(Br)cnc2n1 ; CC(C)OB1OC(C)(C)C(C)(C)O1 ; CC(C)(O)Cn1cc2cc(B(O)O)cnc2n1 ; N#Cc1ccc(-c2cc(C(=O)N3CCC[C@@H](N)C3)sc2Br)cc1F ; "Boc" ; CC(C)(O)Cn1cc2cc(-c3sc(C(=O)N4CCC[C@@H](NC(=O)OC(C)(C)C)C4)cc3-c3ccc(C#N)c(F)c3)cnc2n1]

### Step 1

Under nitrogen atmosphere, compound 60-4 (120 mg, 0.444 mmol), 2-isopropoxy-4,4,5,5-tetramethyl-1,3,2-dioxaborolane (124 mg, 0.666 mmol) were dissolved in anhydrous tetrahydrofuran (5.00 mL), then n-butyllithium (2.5 mol/L n-hexane solution, 711 μL) was added dropwise thereto at −78° C. The reaction mixture was stirred and reacted for 12 hours at 25° C., added with water (20 mL), and extracted with ethyl acetate (20 mL×1). The aqueous phase was concentrated under reduced pressure to obtain compound 61-1. MS-ESI calculated for [M+H]⁺ 236, found 236.

### Step 2

Under nitrogen atmosphere, compound 21-7 (140 mg, 0.275 mmol), compound 61-1 (90.6 mg, 0.386 mmol), and sodium carbonate (87.6 mg, 0.826 mmol) were dissolved in dioxane (10.0 mL) and water (2.00 mL), and the reaction mixture was added with [1,1′-bis(diphenylphosphino)ferrocene]dichloropalladium(II) (20.2 mg, 0.275 mmol). The reaction mixture was stirred and reacted for 2 hours at 80° C., added with water (20 mL), and extracted with ethyl acetate (20 mL×1). The organic phase was washed with saturated brine (20 mL×1), dried over anhydrous sodium sulfate, filtered, and concentrated under reduced pressure. The residue was purified by silica gel column chromatography (1/2, petroleum ether/ethyl acetate, R_{f}=0.19) to obtain compound 61-2. MS-ESI calculated for [M+H]⁺ 619, found 619.

### Step 3

Under nitrogen atmosphere, compound 61-2 (60.0 mg, 0.097 mmol) was dissolved in anhydrous dichloromethane (10.0 mL), and trimethylsilyl trifluoromethanesulfonate (43.1 mg, 0.194 mmol) was added dropwise at 0° C. The reaction mixture was stirred for 1 hour at 25° C., added with saturated sodium bicarbonate aqueous solution (20 mL), and extracted with dichloromethane (20 mL×1). The organic phase was dried over anhydrous sodium sulfate, filtered, concentrated under reduced pressure, and separated by preparative high performance liquid chromatography (chromatographic column: Phenomenex Genimi NX C18 150*40 mm*5 μm; mobile phase: mobile phase A: ammonia aqueous solution with a volume fraction of 0.04% and 10 mmol/L sodium bicarbonate aqueous solution; mobile phase B: acetonitrile; B %: 15 to 45%, 10 minutes) to obtain compound 61. ¹H NMR (400 MHz, CD₃OD) δ 8.40-8.36 (m, 2H), 8.32 (d, J=2.0 Hz, 1H), 7.70-7.64 (m, 1H), 7.59 (s, 1H), 7.40 (d, J=10.4 Hz, 1H), 7.29-7.23 (m, 1H), 4.45 (s, 2H), 4.40-4.33 (m, 1H), 4.30-4.23 (m, 1H), 3.29-3.09 (m, 1H), 3.08-2.94 (m, 1H), 2.92-2.82 (m, 1H), 2.11-2.01 (m, 1H), 1.92-1.84 (m, 1H), 1.69-1.56 (m, 1H), 1.51-1.40 (m, 1H), 1.25 (s, 6H). MS-ESI calculated for [M+H]⁺ 519, found 519.

### Example 62

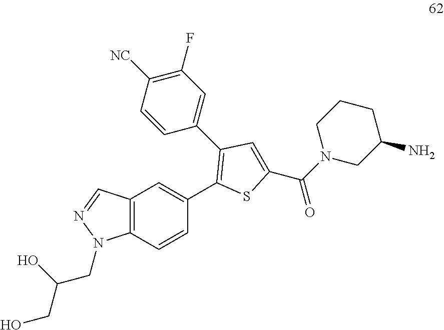 [SMILES: N#Cc1ccc(-c2cc(C(=O)N3CCC[C@@H](N)C3)sc2-c2ccc3c(cnn3CC(O)CO)c2)cc1F]

Synthetic Route:

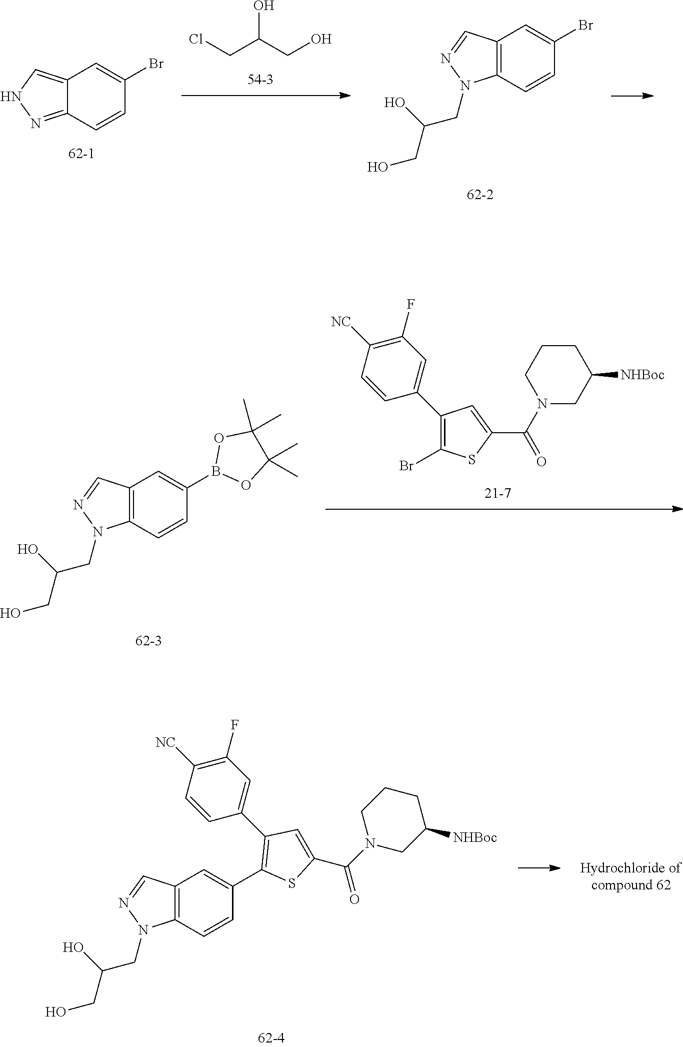 [SMILES: Brc1ccc2n[nH]cc2c1 ; OCC(O)CCl ; OCC(O)Cn1ncc2cc(Br)ccc21 ; CC1(C)OB(c2ccc3c(cnn3CC(O)CO)c2)OC1(C)C ; CC(C)(C)OC(=O)N[C@@H]1CCCN(C(=O)c2cc(-c3ccc(C#N)c(F)c3)c(Br)s2)C1 ; CC(C)(C)OC(=O)N[C@@H]1CCCN(C(=O)c2cc(-c3ccc(C#N)c(F)c3)c(-c3ccc4c(cnn4CC(O)CO)c3)s2)C1]

### Step 1

Compounds 62-1 (4.00 g, 20.3 mmol) and 54-3 (6.73 g, 60.9 mmol) were dissolved in ethanol (30.0 mL), and sodium hydroxide (2.44 g, 60.9 mmol) was added thereto, and the reaction mixture was stirred and reacted for 12 hours at 78° C. The reaction mixture was added with water (100 mL), and extracted with ethyl acetate (100 mL×2). The organic phase was washed with saturated brine (100 mL×1), dried over anhydrous sodium sulfate, filtered, and concentrated under reduced pressure. The residue was purified by silica gel column chromatography (1/2, petroleum ether/ethyl acetate, R_{f}=0.34) to obtain compound 62-2. MS-ESI calculated for [M+H]⁺ 273, found 273.

### Step 2

Under nitrogen atmosphere, compound 62-2 (1.00 g, 3.69 mmol) and bis(pinacolato)diboron (2.81 g, 11.1 mol) were dissolved in dioxane (20.0 mL), and the reaction mixture was added with potassium acetate (1.09 g, 11.1 μmol) and [1,1′-bis(diphenylphosphino)ferrocene]dichloropalladium(II) (269 mg, 369 μmol). The reaction mixture was stirred and reacted at 100° C. for 12 hours. The reaction mixture was concentrated under reduced pressure, and the residue was purified by silica gel column chromatography (1/2, petroleum ether/ethyl acetate, R_{f}=0.31) to obtain compound 62-3. ¹H NMR (400 MHz, CD₃OD) δ=8.22 (s, 1H), 8.09 (s, 1H), 7.76 (d, J=8.4 Hz, 1H), 7.61 (d, J=8.4 Hz, 1H), 4.59-4.50 (m, 1H), 4.49-4.38 (m, 1H), 4.17-4.07 (m, 1H), 3.82-3.71 (m, 1H), 3.47-3.40 (m, 1H), 1.38 (s, 12H). MS-ESI calculated for [M+H]⁺ 319, found 319.

### Step 3

Under nitrogen atmosphere, compound 21-7 (639 mg, 1.26 mmol), compound 62-3 (480 mg, 1.51 mmol), and sodium carbonate (400 mg, 3.77 mmol) were dissolved in dioxane (24.0 mL) and water (6.00 mL), and the reaction mixture was added with [1,1′-bis(diphenylphosphino)ferrocene]dichloropalladium(II) (92.0 mg, 126 μmol), and the reaction mixture was stirred and reacted for 12 hours at 100° C. The reaction mixture was concentrated under reduced pressure, and the residue was purified by silica gel column chromatography (0/1, petroleum ether/ethyl acetate, R_{f}=0.07) to obtain compound 62-4. MS-ESI calculated for [M+H]⁺ 620, found 620.

### Step 4

Compound 62-4 (200 mg, 323 μmol) was dissolved in ethyl acetate (15.0 mL), and hydrochloride ethyl acetate solution (4 mol/L, 807 μL) was added thereto, and the reaction mixture was stirred and reacted for 3 hours at 25° C. After the reaction mixture was concentrated, the residue was purified by preparative high performance liquid chromatography (chromatographic column: Phenomenex Genimi NX C18 150*40 mm*5 μm, mobile phase A: hydrochloric acid aqueous solution with a volume fraction of 0.05%; mobile phase B: acetonitrile; B %: 5% to 35%, 10 minutes) to obtain the hydrochloride of compound 62. ¹H NMR (400 MHz, CD₃OD) δ=8.07 (s, 1H), 7.79 (s, 1H), 7.69-7.59 (m, 3H), 7.33-7.21 (m, 3H), 4.64-4.51 (m, 1H), 4.49-4.37 (m, 2H), 4.24-4.04 (m, 2H), 3.64-3.38 (m, 5H), 2.29-2.15 (m, 1H), 1.97-1.86 (m, 1H), 1.84-1.68 (m, 2H). MS-ESI calculated for [M+H]⁺ 520, found 520.

### Example 63

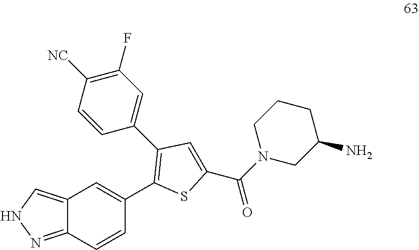 [SMILES: N#Cc1ccc(-c2cc(C(=O)N3CCC[C@@H](N)C3)sc2-c2ccc3n[nH]cc3c2)cc1F]

Synthetic Route:

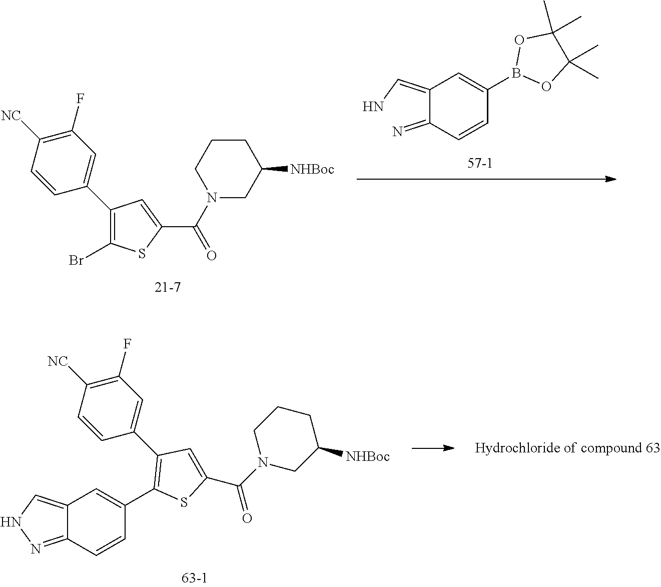 [SMILES: CC(C)(C)OC(=O)N[C@@H]1CCCN(C(=O)c2cc(-c3ccc(C#N)c(F)c3)c(Br)s2)C1 ; CC1(C)OB(C2=C/C(=C/N)C(=N)C=C2)OC1(C)C ; CC(C)(C)OC(=O)N[C@@H]1CCCN(C(=O)c2cc(-c3ccc(C#N)c(F)c3)c(-c3ccc4n[nH]cc4c3)s2)C1]

### Step 1

Under nitrogen atmosphere, compound 21-7 (300 mg, 590 μmol), compound 57-1 (173 mg, 708 μmol), and sodium carbonate (125 mg, 1.18 mmol) were dissolved in dioxane (32.0 mL) and water (8.00 mL), and the reaction mixture was added with [1,1′-bis(diphenylphosphino)ferrocene]dichloropalladium(II) (43.2 mg, 59.0 μmol), and the reaction mixture was stirred and reacted for 12 hours at 100° C. The reaction mixture was concentrated under reduced pressure, and the residue was purified by silica gel column chromatography (0/1, petroleum ether/ethyl acetate, R_{f}=0.18) to obtain compound 63-1. MS-ESI calculated for [M+H]⁺ 546, found 546.

### Step 2

Compound 63-1 (80.0 mg, 147 μmol) was dissolved in ethyl acetate (10.0 mL), and hydrochloride ethyl acetate solution (4 mol/L, 367 μL) was added thereto, and the reaction mixture was stirred and reacted for 12 hours at 25° C. After the reaction mixture was concentrated under reduced pressure, the residue was purified by preparative high performance liquid chromatography (chromatographic column: Xtimate C18 150*40 mm*5 μm, mobile phase A: hydrochloric acid aqueous solution with a volume fraction of 0.05%; mobile phase B: acetonitrile; B %: 8% to 38%, 10 minutes) to obtain the hydrochloride of compound 63. ¹H NMR (400 MHz, CD₃OD) δ=8.13 (s, 1H), 7.86 (s, 1H), 7.71-7.61 (m, 2H), 7.57 (d, J=8.8 Hz, 1H), 7.35 (d, J=10.4 Hz, 1H), 7.29-7.24 (m, 2H), 4.44-4.39 (m, 1H), 4.21-4.16 (m, 1H), 3.65-3.40 (m, 3H), 2.27-2.17 (m, 1H), 2.01-1.88 (m, 1H), 1.84-1.68 (m, 2H). MS-ESI calculated for [M+H]⁺ 446, found 446.

### Example 64

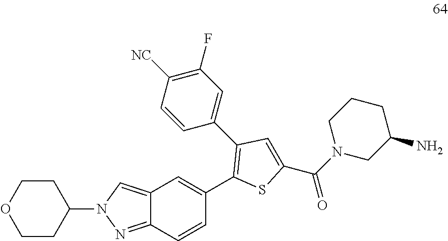 [SMILES: N#Cc1ccc(-c2cc(C(=O)N3CCC[C@@H](N)C3)sc2-c2ccc3nn(C4CCOCC4)cc3c2)cc1F]

Synthetic Route:

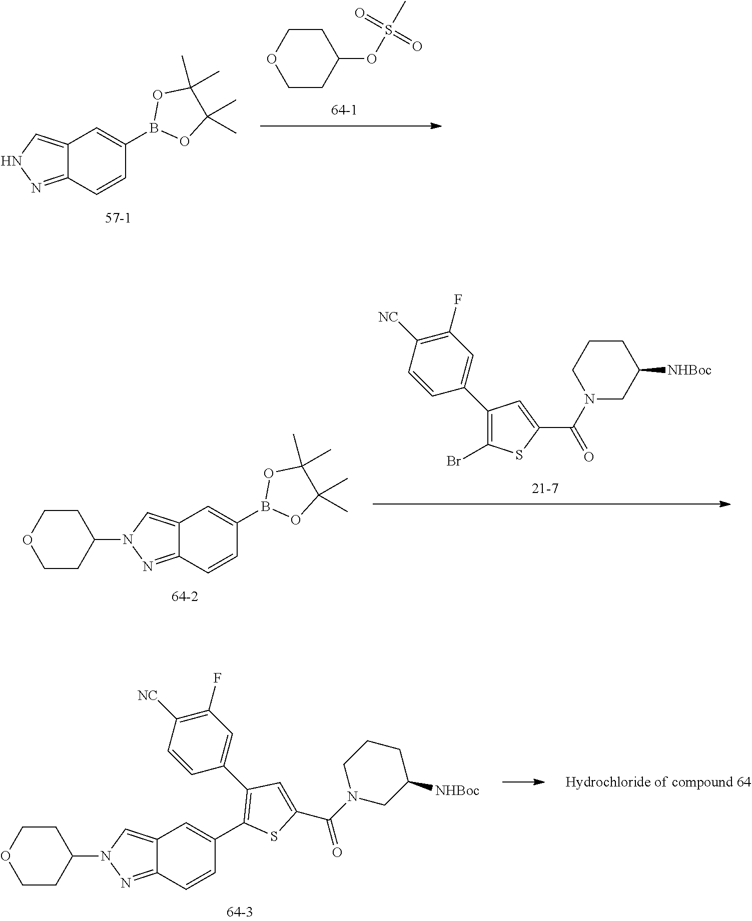 [SMILES: CC1(C)OB(c2ccc3n[nH]cc3c2)OC1(C)C ; CS(=O)(=O)OC1CCOCC1 ; CC1(C)OB(c2ccc3nn(C4CCOCC4)cc3c2)OC1(C)C ; CC(C)(C)OC(=O)N[C@@H]1CCCN(C(=O)c2cc(-c3ccc(C#N)c(F)c3)c(Br)s2)C1 ; CC(C)(C)OC(=O)N[C@@H]1CCCN(C(=O)c2cc(-c3ccc(C#N)c(F)c3)c(-c3ccc4nn(C5CCOCC5)cc4c3)s2)C1]

### Step 1

Compounds 57-1 (400 mg, 1.64 mmol) and 64-1 (886 mg, 4.92 mmol) were dissolved in N,N-dimethylformamide (20.0 mL), and then cesium carbonate (534 mg, 1.64 mmol) was added thereto, and the reaction mixture was stirred and reacted for 12 hours at 100° C. The reaction mixture was added with water (80 mL), and extracted with ethyl acetate (80 mL×2). The organic phase was washed with saturated brine (80 mL×1), dried over anhydrous sodium sulfate, filtered, and concentrated under reduced pressure. The residue was purified by silica gel column chromatography (3/1, petroleum ether/ethyl acetate, R_{f}=0.38) to obtain compound 64-2. MS-ESI calculated for [M+H]⁺ 329, found 329.

### Step 2

Under nitrogen atmosphere, compound 21-7 (150 mg, 295 μmol), compound 64-2 (150 mg, 457 μmol), and sodium carbonate (80.7 mg, 762 μmol) were dissolved in dioxane (4.00 mL) and water (1.00 mL), and the reaction mixture was added with [1,1′-bis(diphenylphosphino)ferrocene]dichloropalladium(II) (27.9 mg, 38.1 μmol), and the reaction mixture was stirred and reacted for 12 hours at 100° C. The reaction mixture was concentrated under reduced pressure, and the residue was purified by silica gel column chromatography (2/1, petroleum ether/ethyl acetate, R_{f}=0.34) to obtain compound 64-3. MS-ESI calculated for [M+H]⁺ 630, found 630.

### Step 3

Compound 64-3 (112 mg, 178 μmol) was dissolved in ethyl acetate (1.00 mL), and hydrochloride ethyl acetate solution (4 mol/L, 445 μL) was added thereto, and the reaction mixture was stirred and reacted for 2 hours at 25° C. After the reaction mixture was concentrated under reduced pressure, the residue was purified by preparative high performance liquid chromatography (chromatographic column: Xtimate C18 150*40 mm*5 μm, mobile phase A: hydrochloric acid aqueous solution with a volume fraction of 0.05%; mobile phase B: acetonitrile; B %: 10% to 40%, 10 minutes) to obtain the hydrochloride of compound 64. ¹H NMR (400 MHz, CD₃OD) δ=8.39 (s, 1H), 7.78 (s, 1H), 7.68-7.58 (m, 3H), 7.34 (d, J=10.4 Hz, 1H), 7.26 (d, J=8.0 Hz, 1H), 7.15 (d, J=8.8 Hz, 1H), 4.45-4.36 (m, 1H), 4.24-4.07 (m, 3H), 3.70-3.58 (m, 2H), 3.59-3.38 (m, 3H), 2.33-2.12 (m, 6H), 1.94-1.89 (m, 1H), 1.83-1.68 (m, 2H). MS-ESI calculated for [M+H]⁺ 530, found 530.

### Example 65

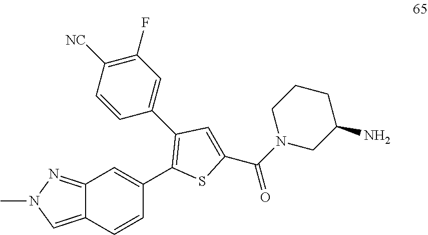 [SMILES: Cn1cc2ccc(-c3sc(C(=O)N4CCC[C@@H](N)C4)cc3-c3ccc(C#N)c(F)c3)cc2n1]

Synthetic Route:

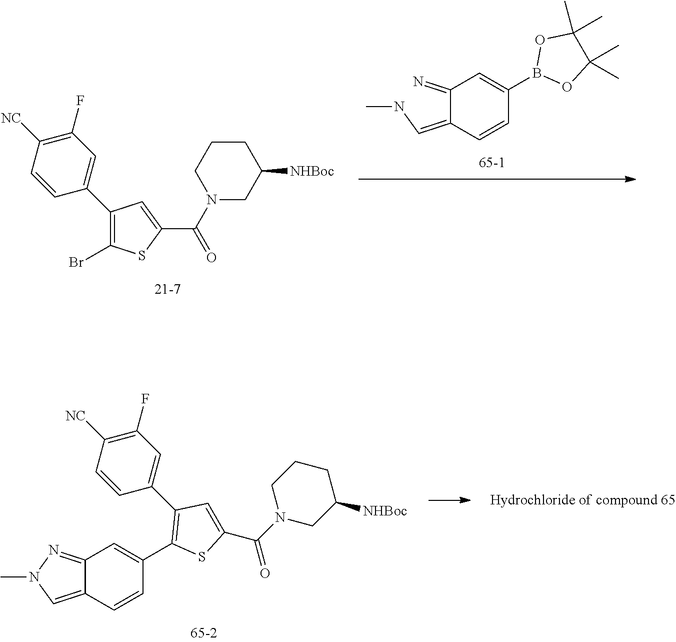

### Step 1

Under nitrogen atmosphere, compound 21-7 (300 mg, 590 μmol), compound 65-1 (228 mg, 885 μmol), and sodium carbonate (125 mg, 1.18 mmol) were dissolved in dioxane (24.0 mL) and water (6.00 mL), and the reaction mixture was added with [1,1′-bis(diphenylphosphino)ferrocene]dichloropalladium(II) (43.2 mg, 59.0 μmol), and the reaction mixture was stirred and reacted for 12 hours at 100° C. The reaction mixture was concentrated under reduced pressure, and the residue was purified by silica gel column chromatography (0/1, petroleum ether/ethyl acetate, R_{f}=0.49) to obtain compound 65-2. MS-ESI calculated for [M−56+H]⁺ 504, found 504.

### Step 2

Compound 65-2 (222 mg, 397 μmol) was dissolved in ethyl acetate (15.0 mL), and hydrochloride ethyl acetate solution (4 mol/L, 992 μL) was added thereto, and the reaction mixture was stirred and reacted for 12 hours at 25° C. After the reaction mixture was concentrated under reduced pressure, the residue was purified by preparative high performance liquid chromatography (chromatographic column: Xtimate C18 150*40 mm*5 μm, mobile phase A: hydrochloric acid aqueous solution with a volume fraction of 0.05%; mobile phase B: acetonitrile; B %: 12% to 52%, 10 minutes) to obtain the hydrochloride of compound 65. ¹H NMR (400 MHz, CD₃OD) δ=8.34 (s, 1H), 7.75 (d, J=8.4 Hz, 1H), 7.69-7.58 (m, 3H), 7.33 (d, J=10.4 Hz, 1H), 7.28 (d, J=8.0 Hz, 1H), 7.00 (d, J=8.8 Hz, 1H), 4.44-4.35 (m, 1H), 4.25 (s, 3H), 4.21-4.10 (m, 1H), 3.62-3.38 (m, 3H), 2.27-2.14 (m, 1H), 1.97-1.86 (m, 1H), 1.84-1.68 (m, 2H). MS-ESI calculated for [M+H]⁺ 460, found 460.

### Example 66

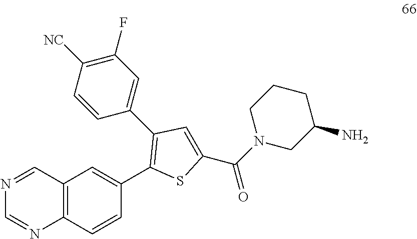 [SMILES: N#Cc1ccc(-c2cc(C(=O)N3CCC[C@@H](N)C3)sc2-c2ccc3ncncc3c2)cc1F]

Synthetic Route:

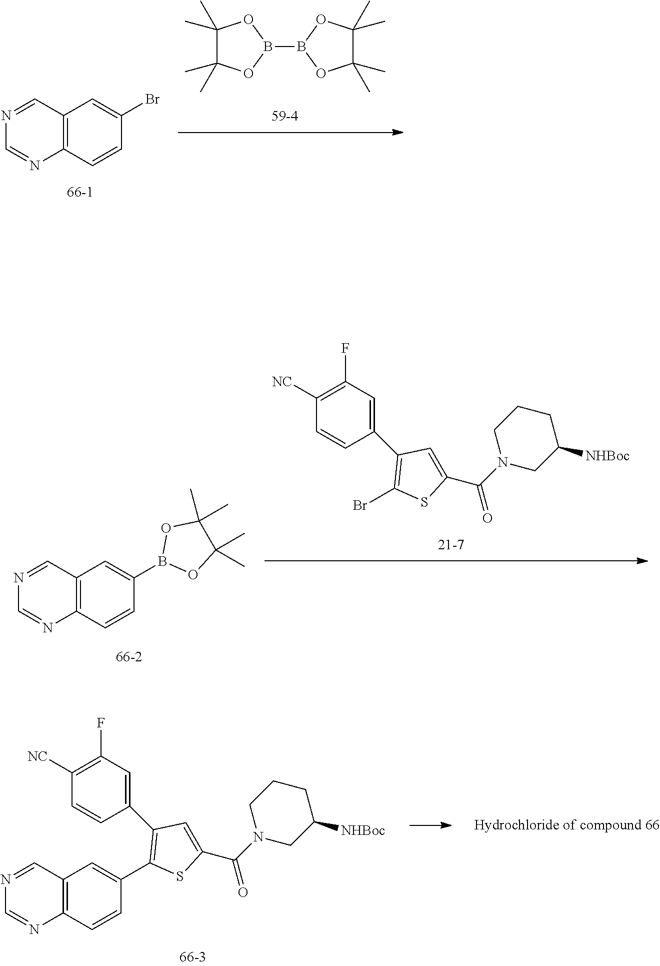

### Step 1

Under nitrogen atmosphere, compound 66-1 (200 mg, 957 μmol), compound 59-4 (292 mg, 1.15 mmol), and potassium acetate (188 mg, 1.91 mmol), and [1,1′-bis(diphenylphosphino)ferrocene]dichloropalladium(II) dichloromethane (70.0 mg, 95.7 μmol) were dissolved in dioxane (5.00 mL). The reaction mixture was stirred and reacted for 12 hours at 90° C. The reaction mixture was concentrated under reduced pressure, and the residue was purified by preparative silica gel thin layer chromatography (1/1, petroleum ether/ethyl acetate, R_{f}=0.50) to obtain compound 66-2. ¹H NMR (400 MHz, CDCl₃) δ=9.37 (s, 1H), 9.29 (s, 1H), 8.39 (s, 1H), 8.04-8.25 (m, 1H), 7.97 (d, J=8.4 Hz, 1H), 1.33 (s, 12H).

### Step 2

Under nitrogen atmosphere, compound 66-2 (121 mg, 472 μmol), compound 21-7 (200 mg, 394 μmol), potassium phosphate (167 mg, 787 μmol), and [1,1′-bis(di-tert-butylphosphino)ferrocene]dichloropalladium(II) (25.6 mg, 39.3 μmol) were dissolved in dioxane (8.00 mL) and water (1.60 mL), and the reaction mixture was stirred and reacted for 12 hours at 90° C. The reaction mixture was filtered through diatomite, and the filtrate was concentrated and purified by preparative silica gel thin layer chromatography (20/1, dichloromethane/methanol, R_{f}=0.24) to obtain compound 66-3. MS-ESI calculated for [M+H]⁺ 558, found 558.

### Step 3

Compound 66-3 (160 mg, 287 μmol) was dissolved in ethyl acetate (700 μL), and hydrochloride ethyl acetate solution (4 mol/L, 287 μL) was added thereto at 0° C., and the reaction mixture was stirred and reacted for 12 hours at 11° C. The reaction mixture was filtrated. The solid was washed with ethyl acetate (400 μL). The residue was purified by preparative high performance liquid chromatography (chromatographic column: 3_Phenomenex Luna C18 150*25 mm*10 μm, mobile phase A: hydrochloric acid aqueous solution with a volume fraction of 0.05%; mobile phase B: acetonitrile; B %: 3% to 33%, 10 minutes) to obtain the hydrochloride of compound 66. ¹H NMR (400 MHz, DMSO-d₆) δ=9.65 (s, 1H), 9.34 (s, 1H), 8.28-8.24 (m, 4H), 7.99 (d, J=8.8 Hz, 1H), 7.84 (t, J=8.8 Hz, 1H), 7.80-7.77 (m, 1H), 7.74 (s, 1H), 7.70-7.67 (m, 1H), 7.27-7.24 (m, 1H), 4.36-4.34 (m, 1H), 4.09-4.05 (m, 1H), 3.37-3.23 (m, 3H), 2.08-2.04 (m, 1H), 1.87-1.82 (m, 1H), 1.69-1.57 (m, 2H). MS-ESI calculated for [M+H]⁺ 458, found 458.

### Example 67

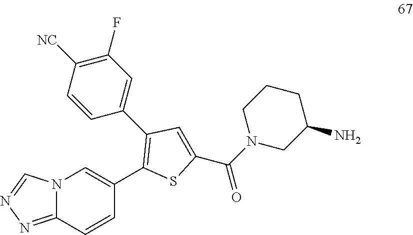 [SMILES: N#Cc1ccc(-c2cc(C(=O)N3CCC[C@@H](N)C3)sc2-c2ccc3nncn3c2)cc1F]

Synthetic Route:

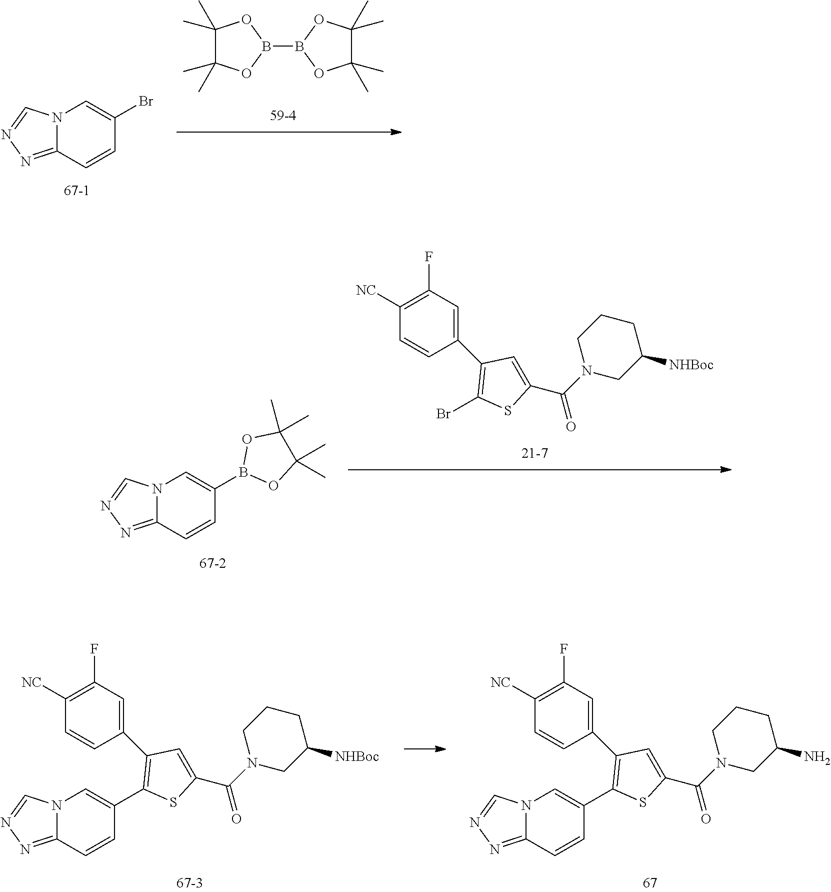

### Step 1

Under nitrogen atmosphere, compound 67-1 (95.0 mg, 984 μmol), compound 59-4 (300 mg, 1.18 mmol), potassium acetate (193 mg, 1.97 mmol), and [1,1′-bis(diphenylphosphino)ferrocene]dichloropalladium(II) dichloromethane (80.4 mg, 98.5 μmol) were dissolved in dioxane (7.00 mL). The reaction mixture was stirred and reacted for 12 hours at 85° C. The reaction mixture was filtered through diatomite, and the filtrate was concentrated to obtain crude compound 67-2, which was used directly in the next reaction step.

### Step 2

Under nitrogen atmosphere, compound 67-2 (240 mg, 979 μmol), compound 21-7 (527 mg, 979 μmol), potassium phosphate (416 mg, 1.96 mmol), and [1,1′-bis(di-tert-butylphosphino)ferrocene]dichloropalladium(II) (63.8 mg, 97.9 μmol) were dissolved in dioxane (8.00 mL) and water (1.60 mL), and the reaction mixture was stirred and reacted for 12 hours at 85° C. The reaction mixture was filtered through diatomite, and the filtrate was concentrated and purified by preparative silica gel thin layer chromatography (20/1, dichloromethane/methanol, R_{f}=0.26) to obtain compound 67-3. MS-ESI calculated for [M+H]⁺ 547, found 547.

### Step 3

Compound 67-3 (240 mg, 363 μmol) was dissolved in ethyl acetate (6.00 mL), and hydrochloride ethyl acetate solution (4 mol/L, 342 μL) was added thereto at 0° C., and the reaction mixture was stirred and reacted for 12 hours at 11° C. The reaction mixture was filtered. The solid was washed with ethyl acetate (400 μL), and the residue was purified by preparative high performance liquid chromatography (chromatographic column: 3_Phenomenex Luna C18 75*30 mm*3 μm; mobile phase: mobile phase A: hydrochloric acid aqueous solution with a volume fraction of 0.05%; mobile phase B: acetonitrile; B %: 11% to 31%, 6 minutes); the purified solution was adjusted to pH=9 to 10 with ammonia water, extracted with dichloromethane/methanol (10/1, 90 mL), dried over anhydrous sodium sulfate, filtered, and concentrated to obtain compound 67. ¹H NMR (400 MHz, CD₃OD) δ=9.19 (s, 1H), 8.70-8.69 (m, 1H), 7.72-7.69 (m, 2H), 7.61 (s, 1H), 7.50-7.48 (m, 1H), 7.34-7.31 (m, 1H), 7.22-7.19 (m, 1H), 4.35-4.29 (m, 1H), 4.26-4.21 (m, 1H), 3.25-3.16 (m, 1H), 3.05-3.00 (m, 1H), 2.91-2.84 (m, 1H), 2.08-2.04 (m, 1H), 1.90-1.84 (m, 1H), 1.67-1.57 (m, 1H), 1.50-1.40 (m, 1H). MS-ESI calculated for [M+H]⁺ 447, found 447.

### Example 68

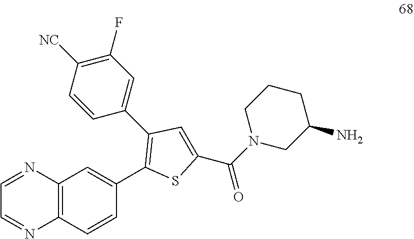 [SMILES: N#Cc1ccc(-c2cc(C(=O)N3CCC[C@@H](N)C3)sc2-c2ccc3nccnc3c2)cc1F]

Synthetic Route:

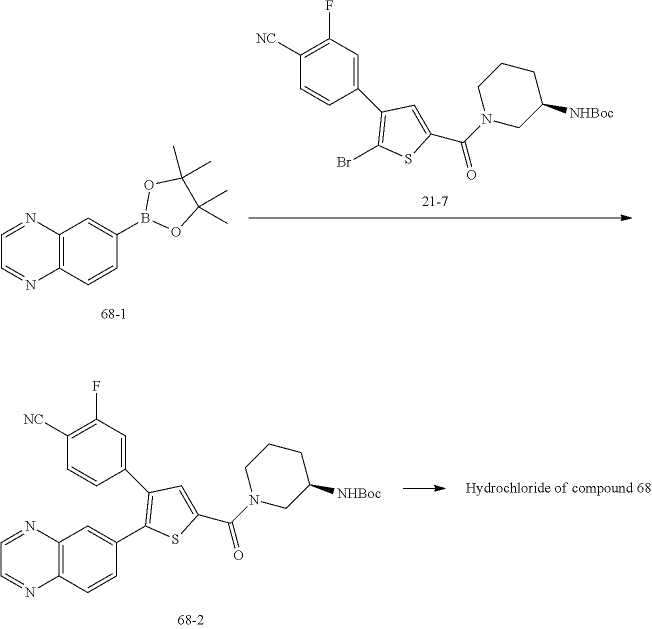 [SMILES: CC1(C)OB(c2ccc3nccnc3c2)OC1(C)C ; CC(C)(C)OC(=O)N[C@@H]1CCCN(C(=O)c2cc(-c3ccc(C#N)c(F)c3)c(Br)s2)C1 ; CC(C)(C)OC(=O)N[C@@H]1CCCN(C(=O)c2cc(-c3ccc(C#N)c(F)c3)c(-c3ccc4nccnc4c3)s2)C1]

### Step 1

Under nitrogen atmosphere, compound 68-1 (121 mg, 471 μmol), compound 21-7 (212 mg, 394 μmol), potassium phosphate (167 mg, 785 μmol), and [1,1′-bis(di-tert-butylphosphino)ferrocene]dichloropalladium(II) (25.6 mg, 39.2 μmol) were dissolved in dioxane (8.00 mL) and water (1.60 mL), and the reaction mixture was stirred and reacted for 12 hours at 80° C. The reaction mixture was filtered through diatomite, and the filtrate was concentrated and purified by preparative silica gel thin layer chromatography (20/1, dichloromethane/methanol, R_{f}=0.25) to obtain compound 68-2. ¹H NMR (400 MHz, CDCl₃) δ=8.88 (s, 2H), 8.16-8.12 (m, 1H), 8.06 (d, J=8.4 Hz, 1H), 7.83 (s, 1H), 7.64-7.59 (m, 2H), 7.45 (t, J=7.2 Hz, 1H), 7.24 (d, J=8.0 Hz, 1H), 4.72-4.59 (m, 2H), 4.48-4.39 (m, 1H), 3.75-3.71 (m, 1H), 3.05-2.93 (m, 2H), 2.12-2.09 (m, 1H), 1.93-1.88 (m, 1H), 1.75-1.66 (m, 1H), 1.48 (s, 9H). MS-ESI calculated for [M+H]⁺ 558, found 558.

### Step 2

Compound 68-2 (180 mg, 317 μmol) was dissolved in ethyl acetate (1.50 mL), and hydrochloride ethyl acetate solution (4 mol/L, 317 μL) was added thereto at 0° C., and the reaction mixture was stirred and reacted for 12 hours at 11° C. The reaction mixture was filtered. The solid was washed with ethyl acetate (0.4 mL), and the residue was purified by preparative high performance liquid chromatography (chromatographic column: 3_Phenomenex Luna C18 75*30 mm*3 μm; mobile phase: mobile phase A: hydrochloric acid aqueous solution with a volume fraction of 0.05%; mobile phase B: acetonitrile; B %: 0% to 40%, 6.5 minutes) to obtain the hydrochloride of compound 68. ¹H NMR (400 MHz, DMSO-d₆) δ=8.99 (s, 2H), 8.34-8.29 (m, 3H), 8.10-8.08 (m, 2H), 7.89-7.84 (m, 1H), 7.74 (s, 1H), 7.72-7.65 (m, 2H), 4.37-4.35 (m, 1H), 4.10-4.06 (m, 1H), 3.38-3.33 (m, 3H), 2.09-2.05 (m, 1H), 1.86-1.83 (m, 1H), 1.70-1.57 (m, 2H). MS-ESI calculated for [M+H]⁺ 458, found 458.

### Example 69

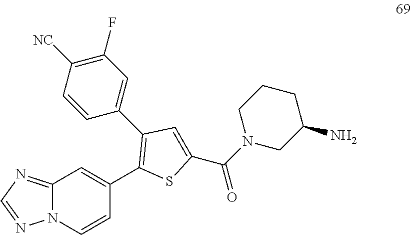 [SMILES: N#Cc1ccc(-c2cc(C(=O)N3CCC[C@@H](N)C3)sc2-c2ccn3ncnc3c2)cc1F]

Synthetic Route:

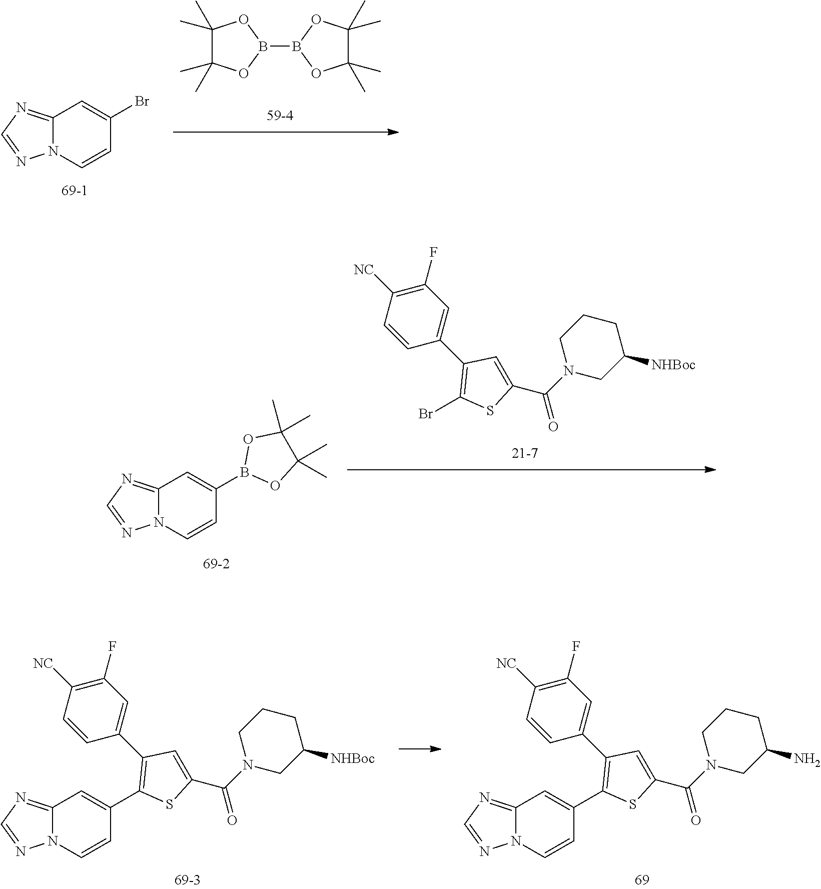

### Step 1

Under nitrogen atmosphere, compound 69-1 (117 mg, 599 μmol), compound 59-4 (180 mg, 709 μmol), and potassium acetate (116 mg, 1.18 mmol), and [1,1′-bis(diphenylphosphino)ferrocene]dichloropalladium(II) dichloromethane (48.3 mg, 59.1 μmol) were dissolved in dioxane (4.00 mL). The reaction mixture was stirred and reacted for 12 hours at 85° C. The reaction mixture was filtered through diatomite, and the filtrate was concentrated to obtain crude compound 69-2, which was used directly in the next reaction step.

### Step 2

Under nitrogen atmosphere, compounds 69-2 (150 mg, 612 μmol), 21-7 (323 mg, 612 μmol), potassium phosphate (260 mg, 1.22 mmol), and [1,1′-bis(di-tert-butylphosphino)ferrocene]dichloropalladium(II) (39.9 mg, 61.2 μmol) were dissolved in dioxane (8.00 mL) and water (1.60 mL), and the reaction mixture was stirred and reacted for 12 hours at 90° C. The reaction mixture was filtered through diatomite, and the filtrate was concentrated and purified by preparative silica gel thin layer chromatography (20/1, dichloromethane/methanol, R_{f}=0.26) to obtain compound 69-3. MS-ESI calculated for [M+H]⁺ 547, found 547.

### Step 3

Compound 69-3 (210 mg, 358 μmol) was dissolved in ethyl acetate (6.00 mL), and hydrochloride ethyl acetate solution (4 mol/L, 287 μL) was added thereto at 0° C., and the reaction mixture was stirred and reacted for 12 hours at 11° C. The reaction mixture was filtered. The solid was washed with ethyl acetate (0.4 mL), and the residue was purified by preparative high performance liquid chromatography (chromatographic column: 3_Phenomenex Luna C18 75*30 mm*3 μm; mobile phase: mobile phase A: hydrochloric acid aqueous solution with a volume fraction of 0.05%; mobile phase B: acetonitrile; B %: 21% to 31%, 6 minutes); the purified solution was adjusted to pH=9 to 10 with ammonia water, extracted with dichloromethane/methanol (10/1, 70 mL), dried over anhydrous sodium sulfate, filtered, and concentrated to obtain compound 69. ¹H NMR (400 MHz, DMSO-d₆) δ=8.94-8.90 (m, 1H), 8.57 (s, 1H), 7.91-7.87 (m, 2H), 7.70-7.67 (m, 2H), 7.34-7.32 (m, 1H), 6.95-6.93 (m, 1H), 4.19-4.06 (m, 2H), 3.30 (s, 2H), 3.18-3.04 (m, 1H), 2.78-2.72 (m, 1H), 1.90-1.87 (m, 1H), 1.79-1.73 (m, 2H), 1.53-1.43 (m, 1H), 1.34-1.24 (m, 1H). MS-ESI calculated for [M+H]⁺ 447, found 447.

### Example 70

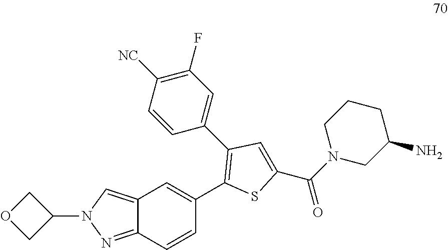 [SMILES: N#Cc1ccc(-c2cc(C(=O)N3CCC[C@@H](N)C3)sc2-c2ccc3nn(C4COC4)cc3c2)cc1F]

Synthetic Route:

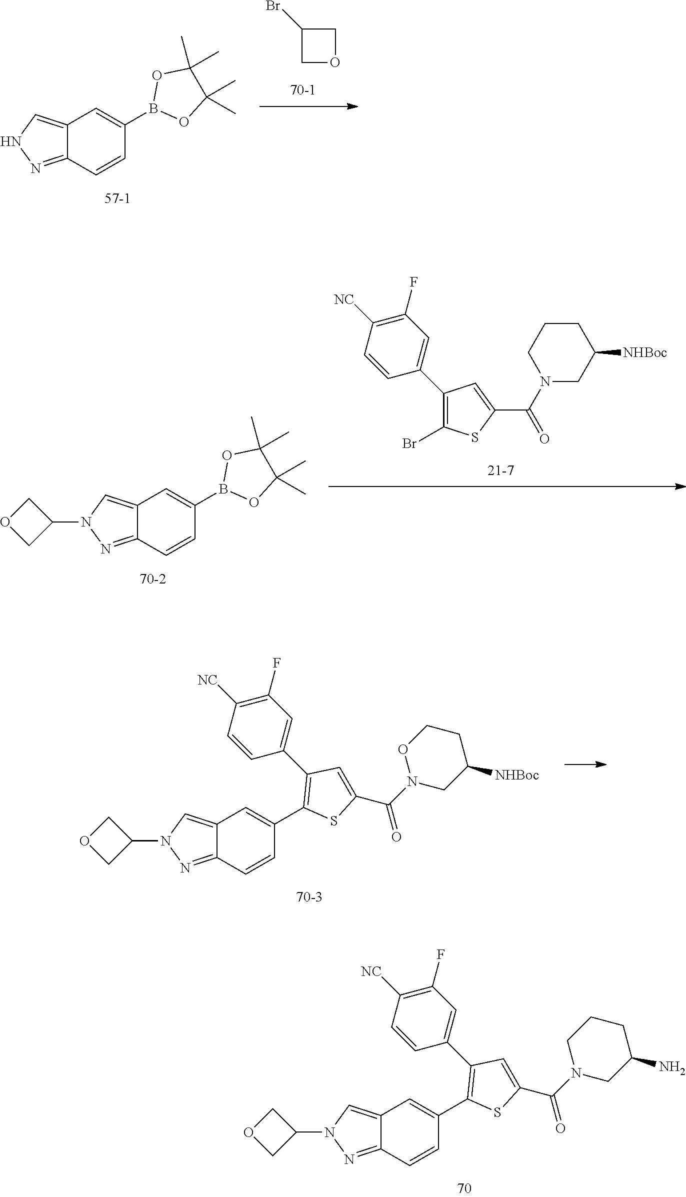 [SMILES: CC1(C)OB(c2ccc3n[nH]cc3c2)OC1(C)C ; BrC1COC1 ; CC1(C)OB(c2ccc3nn(C4COC4)cc3c2)OC1(C)C ; CC(C)(C)OC(=O)N[C@@H]1CCCN(C(=O)c2cc(-c3ccc(C#N)c(F)c3)c(Br)s2)C1 ; CC(C)(C)OC(=O)N[C@@H]1CCON(C(=O)c2cc(-c3ccc(C#N)c(F)c3)c(-c3ccc4nn(C5COC5)cc4c3)s2)C1 ; N#Cc1ccc(-c2cc(C(=O)N3CCC[C@@H](N)C3)sc2-c2ccc3nn(C4COC4)cc3c2)cc1F]

### Step 1

Compounds 57-1 (1.45 g, 5.94 mmol) and 70-1 (2.44 g, 17.8 mmol) were dissolved in N,N-dimethylformamide (30.0 mL), and then potassium carbonate (2.46 g, 17.8 mmol) was added thereto, and the reaction mixture was stirred and reacted for 12 hours at 100° C. The reaction mixture was added with water (150 mL), and extracted with ethyl acetate (150 mL×2). The organic phase was washed with saturated brine (150 mL×1), dried over anhydrous sodium sulfate, filtered, and concentrated under reduced pressure. The residue was purified by silica gel column chromatography (3/1, petroleum ether/ethyl acetate, R_{f}=0.42) to obtain compound 70-2. MS-ESI calculated for [M+H]⁺ 301, found 301.

### Step 2

Under nitrogen atmosphere, compound 21-7 (291 mg, 572 μmol), compound 70-2 (340 mg, 686 μmol), and sodium carbonate (182 mg, 1.72 mmol) were dissolved in dioxane (24.0 mL) and water (6.00 mL), and the reaction mixture was added with [1,1′-bis(diphenylphosphino)ferrocene]dichloropalladium(II) (41.9 mg, 57.2 μmol), and the reaction mixture was stirred and reacted for 12 hours at 100° C. The reaction mixture was concentrated under reduced pressure, and the residue was purified by silica gel column chromatography (1/2, petroleum ether/ethyl acetate, R_{f}=0.32) to obtain compound 70-3. MS-ESI calculated for [M+H]⁺ 602, found 602.

### Step 3

Compound 70-3 (100 mg, 166 μmol) was dissolved in dichloromethane (1.00 mL), and trifluoroacetic acid (1.00 mL) was added thereto, and the reaction mixture was stirred and reacted for 1 hour at 25° C. The reaction mixture was concentrated, added with water (3.00 mL), and then adjusted to pH=8 with ammonia water. The residue was purified by preparative high performance liquid chromatography (chromatographic column: Phenomenex Gemini-NX 80*30 mm*3 μm, mobile phase A: mobile phase A: 10 mM ammonium bicarbonate aqueous solution; mobile phase B: acetonitrile; B %: 30% to 60%, 9 minutes) to obtain compound 70. ¹H NMR (400 MHz, CD₃OD) δ=8.41 (s, 1H), 7.78 (s, 1H), 7.70-7.60 (m, 2H), 7.56 (s, 1H), 7.33 (d, J=10.4 Hz, 1H), 7.27 (dd, J=1.2, 8.0 Hz, 1H), 7.15 (dd, J=1.2, 8.8 Hz, 1H), 5.90-5.83 (m, 1H), 5.20-5.13 (m, 4H), 4.40-4.33 (m, 1H), 4.30-4.24 (m, 1H), 3.25-3.10 (m, 1H), 3.06-2.93 (m, 1H), 2.92-2.81 (m, 1H), 2.12-2.01 (m, 1H), 1.91-1.81 (m, 1H), 1.71-1.56 (m, 1H), 1.53-1.38 (m, 1H). MS-ESI calculated for [M+H]⁺ 502, found 502.

### Example 71

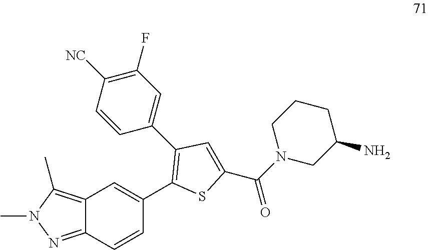 [SMILES: Cc1c2cc(-c3sc(C(=O)N4CCC[C@@H](N)C4)cc3-c3ccc(C#N)c(F)c3)ccc2nn1C]

Synthetic Route:

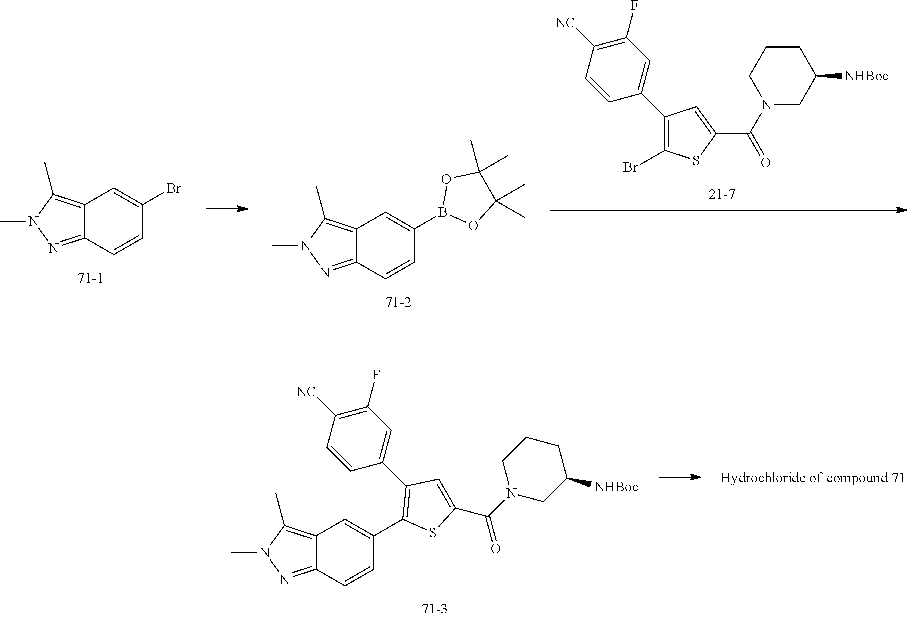

### Step 1

Under nitrogen atmosphere, compound 71-1 (1.00 g, 4.44 mmol) and bis(pinacolato)diboron (3.38 g, 13.3 mmol), and potassium acetate (1.31 g, 13.3 mmol) were dissolved in dioxane (30.0 mL), and the reaction mixture was added with [1,1′-bis(diphenylphosphino)ferrocene]dichloropalladium(II) (325 mg, 444 μmol). The reaction mixture was stirred and reacted at 100° C. for 12 hours. The reaction mixture was concentrated under reduced pressure, and the residue was purified by silica gel column chromatography (2/1, petroleum ether/ethyl acetate, R_{f}=0.37) to obtain compound 71-2. ¹H NMR (400 MHz, CD₃OD) δ=8.13 (s, 1H), 7.58 (dd, J=0.8, 8.8 Hz, 1H), 7.45 (dd, J=0.8, 8.8 Hz, 1H), 4.08 (s, 3H), 2.65 (s, 3H), 1.36 (s, 12H). MS-ESI calculated for [M+H]⁺ 273, found 273.

### Step 2

Under nitrogen atmosphere, compound 21-7 (467 mg, 918 μmol), compound 71-2 (300 mg, 1.10 mmol), and sodium carbonate (195 mg, 1.84 mmol) were dissolved in dioxane (24.0 mL) and water (6.00 mL), and the reaction mixture was added with [1,1′-bis(diphenylphosphino)ferrocene]dichloropalladium(II) (67.2 mg, 91.9 μmol), and the reaction mixture was stirred and reacted for 12 hours at 100° C. The reaction mixture was concentrated under reduced pressure, and the residue was purified by silica gel column chromatography (1/2, petroleum ether/ethyl acetate, R_{f}=0.32) to obtain compound 71-3. MS-ESI calculated for [M+H]⁺ 574, found 574.

### Step 3

Compound 71-3 (325 mg, 566 μmol) was dissolved in ethyl acetate (5.00 mL), and hydrochloride ethyl acetate solution (4 mol/L, 1.42 mL) was added thereto, and the reaction mixture was stirred and reacted for 1 hour at 25° C. After the reaction mixture was concentrated, the residue was purified by preparative high performance liquid chromatography (chromatographic column: Xtimate C18 150*40 mm*5 μm, mobile phase A: hydrochloric acid aqueous solution with a volume fraction of 0.05%; mobile phase B: acetonitrile; B %: 10% to 50%, 10 minutes) to obtain the hydrochloride of compound 71. ¹H NMR (400 MHz, CD₃OD) δ=8.06 (s, 1H), 7.69-7.59 (m, 3H), 7.44 (d, J=8.8 Hz, 1H), 7.36-7.26 (m, 2H), 4.48-4.41 (m, 1H), 4.25 (s, 3H), 4.25-4.16 (m, 1H), 3.65-3.38 (m, 3H), 2.79 (s, 3H), 2.30-2.14 (m, 1H), 2.00-1.89 (m, 1H), 1.87-1.67 (m, 2H). MS-ESI calculated for [M+H]⁺ 474, found 474.

### Example 72

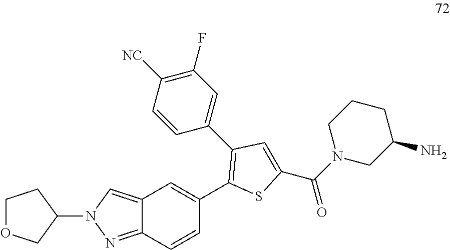 [SMILES: N#Cc1ccc(-c2cc(C(=O)N3CCC[C@@H](N)C3)sc2-c2ccc3nn(C4CCOC4)cc3c2)cc1F]

Synthetic Route:

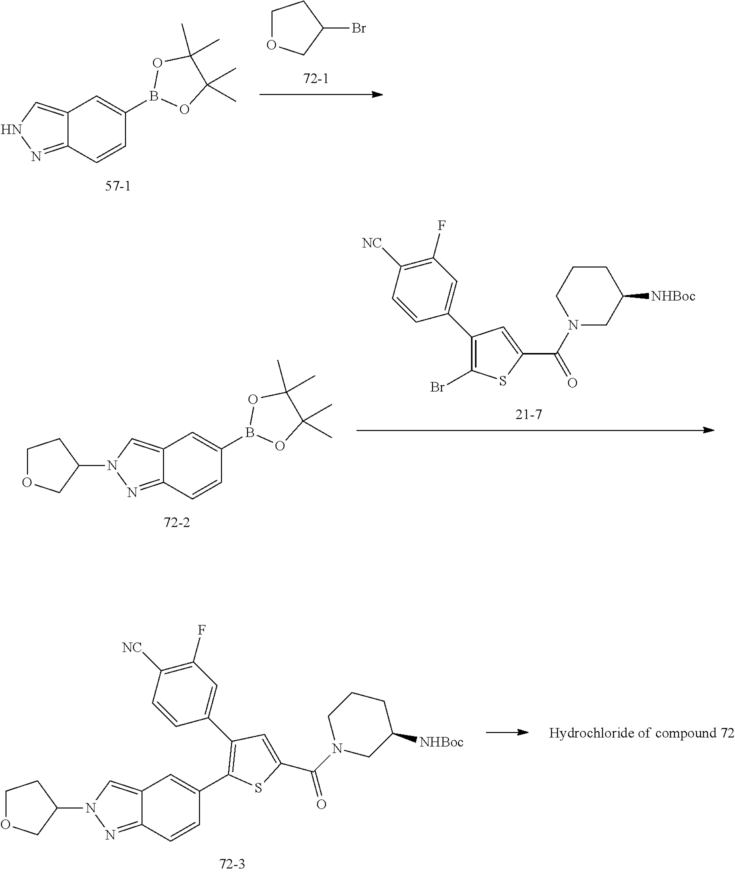

### Step 1

Compounds 57-1 (862 mg, 3.53 mmol) and 72-1 (1.60 g, 10.6 mmol) were dissolved in N,N-dimethylformamide (20.0 mL), and then potassium carbonate (1.46 g, 10.6 mmol) was added thereto, and the reaction mixture was stirred and reacted for 12 hours at 100° C. The reaction mixture was added with water (150 mL), and extracted with ethyl acetate (150 mL×2). The organic phase was washed with saturated brine (150 mL×1), dried over anhydrous sodium sulfate, filtered, and concentrated under reduced pressure. The residue was purified by silica gel column chromatography (2/1, petroleum ether/ethyl acetate, R_{f}=0.19) to obtain compound 72-2. ¹H NMR (400 MHz, CD₃OD) δ=8.35 (s, 1H), 8.20 (s, 1H), 7.63-7.53 (m, 2H), 5.37-5.28 (m, 1H), 4.28-4.10 (m, 3H), 4.01-3.91 (m, 1H), 2.67-2.54 (m, 1H), 2.50-2.39 (m, 1H), 1.36 (s, 12H). MS-ESI calculated for [M+H]⁺ 315, found 315.

### Step 2

Under nitrogen atmosphere, compound 21-7 (351 mg, 690 μmol), compound 72-2 (260 mg, 828 μmol), and sodium carbonate (146 mg, 1.38 mmol) were dissolved in dioxane (16.0 mL) and water (4.00 mL), and the reaction mixture was added with [1,1′-bis(diphenylphosphino)ferrocene]dichloropalladium(II) (50.5 mg, 69.0 μmol), and the reaction mixture was stirred and reacted for 12 hours at 100° C. The reaction mixture was concentrated under reduced pressure, and the residue was purified by silica gel column chromatography (1/2, petroleum ether/ethyl acetate, R_{f}=0.32) to obtain compound 72-3. MS-ESI calculated for [M+H]⁺ 616, found 616.

### Step 3

Compound 72-3 (340 mg, 552 μmol) was dissolved in ethyl acetate (5.00 mL), and hydrochloride ethyl acetate solution (4 mol/L, 1.38 mL) was added thereto, and the reaction mixture was stirred and reacted for 2 hours at 25° C. After the reaction mixture was concentrated, the residue was purified by preparative high performance liquid chromatography (chromatographic column: Xtimate C18 150*40 mm*5 μm, mobile phase A: hydrochloric acid aqueous solution with a volume fraction of 0.05%; mobile phase B: acetonitrile; B %: 5% to 50%, 10 minutes) to obtain the hydrochloride of compound 72. ¹H NMR (400 MHz, CD₃OD) δ=8.38 (s, 1H), 7.79 (s, 1H), 7.70-7.58 (m, 3H), 7.34 (d, J=10.4 Hz, 1H), 7.28 (d, J=8.4 Hz, 1H), 7.14 (d, J=8.8 Hz, 1H), 5.41-5.32 (m, 1H), 4.45-4.42 (m, 1H), 4.29-4.13 (m, 4H), 4.01-3.95 (m, 1H), 3.61-3.39 (m, 3H), 2.72-2.59 (m, 1H), 2.54-2.41 (m, 1H), 2.45-2.21 (m, 1H), 1.99-1.89 (m, 1H), 1.87-1.70 (m, 2H). MS-ESI calculated for [M+H]⁺ 516, found 516.

### Example 73

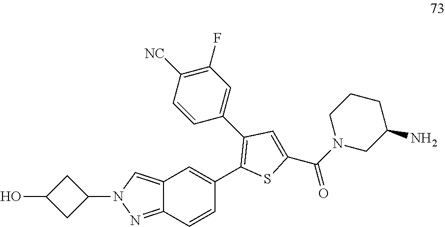 [SMILES: N#Cc1ccc(-c2cc(C(=O)N3CCC[C@@H](N)C3)sc2-c2ccc3nn(C4CC(O)C4)cc3c2)cc1F]

Synthetic Route:

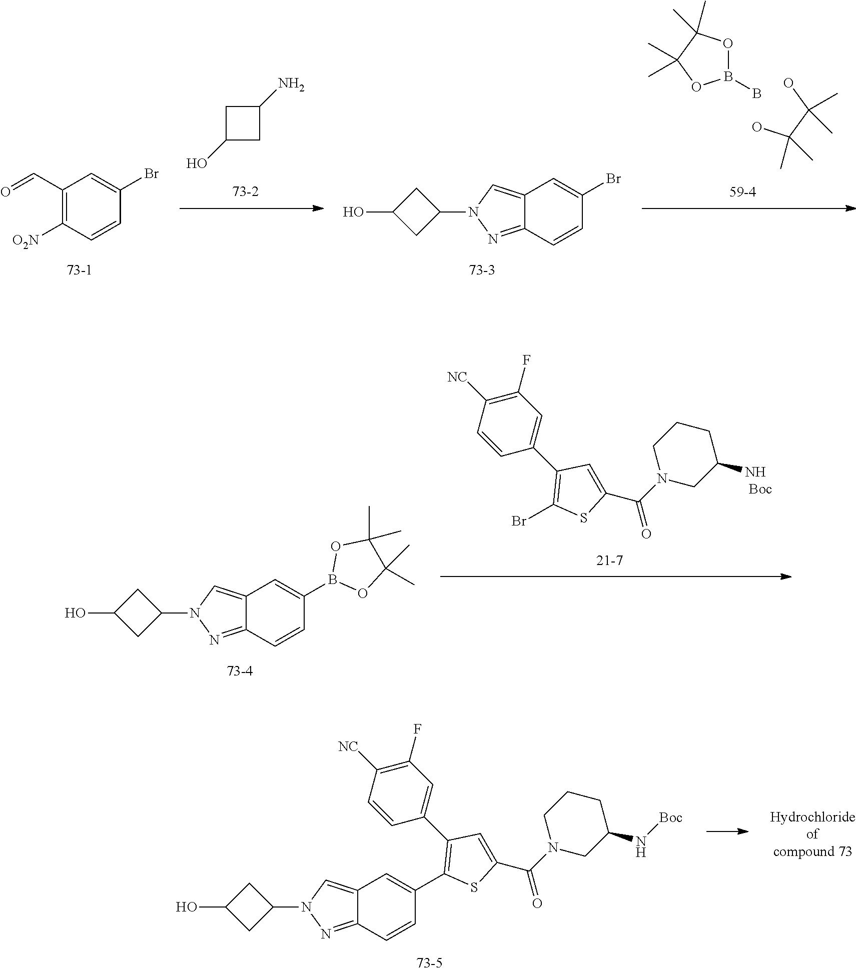

### Step 1

Under nitrogen atmosphere, compound 73-1 (100 mg, 0.435 mmol) was dissolved in isopropanol (10.0 mL), and 3-aminocyclobutanol (41.7 mg, 0.478 mmol) was added thereto, and the reaction mixture was stirred for 4 hours at 80° C., added with tributylphosphine (264 mg, 1.30 mmol) at 0° C., stirred for 12 hours at 80° C., added with saturated ammonium chloride (30 mL), and extracted with ethyl acetate (30 mL×1). The organic phase was washed with saturated brine (30 mL×1), dried over anhydrous sodium sulfate, filtered, concentrated under reduced pressure, and the residue was purified by silica gel column chromatography (1/1, petroleum ether/ethyl acetate, R_{f}=0.12) to obtain compound 73-3. ¹H NMR (400 MHz, CDCl₃) δ=7.96-7.87 (m, 1H), 7.85-7.77 (m, 1H), 7.65-7.56 (m, 1H), 7.41-7.32 (m, 1H), 5.31-5.19 (m, 0.4H), 4.92-4.80 (m, 0.4H), 4.74-4.62 (m, 0.6H), 4.35-4.26 (m, 0.6H), 3.16-2.97 (m, 2H), 2.70-2.62 (m, 2H). MS-ESI calculated for [M+H]⁺ 267, found 267.

### Step 2

Under nitrogen atmosphere, compound 73-3 (100 mg, 0.374 mmol), bis(pinacolato)diboron (285 mg, 1.12 mmol), and potassium acetate (184 mg, 1.87 mmol) were dissolved in dioxane (10.0 mL), and the reaction mixture was added with [1,1′-bis(diphenylphosphino)ferrocene]dichloropalladium(II) (54.8 mg, 74.9 μmol), stirred and reacted for 12 hours at 95° C., and concentrated under reduced pressure. The residue was purified by silica gel column chromatography (1/1, petroleum ether/ethyl acetate, R_{f}=0.60) to obtain compound 73-4. ¹H NMR (400 MHz, CDCl₃) δ=8.23 (s, 1H), 8.02-7.96 (m, 1H), 7.71-7.66 (m, 2H), 5.31-5.23 (m, 0.4H), 4.92-4.83 (m, 0.4H), 4.76-4.68 (m, 0.6H), 4.36-4.28 (m, 0.6H), 3.19-2.99 (m, 2H), 2.75-2.61 (m, 2H), 1.37 (s, 12H). MS-ESI calculated for [M+H]⁺ 315, found 315.

### Step 3

Under nitrogen atmosphere, compound 21-7 (145 mg, 0.285 mmol), compound 73-4 (98.6 mg, 0.314 mmol), and sodium carbonate (90.7 mg, 0.856 mmol) were dissolved in dioxane (8.00 mL) and water (2.00 mL), and the reaction mixture was added with [1,1′-bis(di-tert-butylphosphino)ferrocene]dichloropalladium(II) (18.6 mg, 28.5 μmol). The reaction mixture was stirred and reacted for 2 hours at 80° C., added with water (20 mL), and extracted with ethyl acetate (20 mL×1). The organic phase was washed with saturated brine (20 mL×1), dried over anhydrous sodium sulfate, filtered, and concentrated under reduced pressure, and the residue was purified by silica gel column chromatography (1/1, petroleum ether/ethyl acetate, R_{f}=0.21) to obtain compound 73-5. MS-ESI calculated for [M+H]⁺ 616, found 616.

### Step 4

Under nitrogen atmosphere, compound 73-5 (61.0 mg, 99.1 μmol) was dissolved in anhydrous dichloromethane (5.00 mL), and trimethylsilyl trifluoromethanesulfonate (44.0 mg, 198 μmol) was added dropwise at 0° C. The reaction mixture was stirred for 1 hour at 25° C., added with water (1 mL), concentrated under reduced pressure, and then separated by preparative high performance liquid chromatography (chromatographic column: Phenomenex Genimi NX C18 150*40 mm*5 μm; mobile phase: mobile phase A: hydrochloric acid aqueous solution with a volume fraction of 0.05%; mobile phase B: acetonitrile; B %: 5 to 35%, 10 minutes) to obtain the hydrochloride of compound 73. ¹H NMR (400 MHz, CD₃OD) δ=8.49-8.43 (m, 1H), 7.81 (s, 1H), 7.68-7.58 (m, 3H), 7.38-7.17 (m, 3H), 5.38-5.28 (m, 0.4H), 4.76-4.68 (m, 1H), 4.45-4.35 (m, 1H), 4.27-4.19 (m, 1H), 4.18-4.14 (m, 0.6H), 3.59-3.38 (m, 3H), 3.07-2.89 (m, 2H), 2.70-2.55 (m, 2H), 2.25-2.15 (m, 1H), 1.98-1.87 (m, 1H), 1.86-1.68 (m, 2H). MS-ESI calculated for [M+H]⁺ 516, found 516.

### Example 74

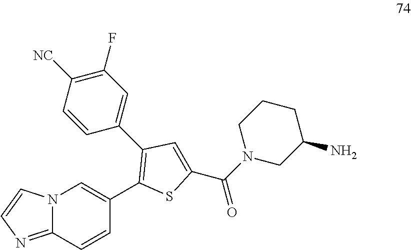 [SMILES: N#Cc1ccc(-c2cc(C(=O)N3CCC[C@@H](N)C3)sc2-c2ccc3nccn3c2)cc1F]

Synthetic Route:

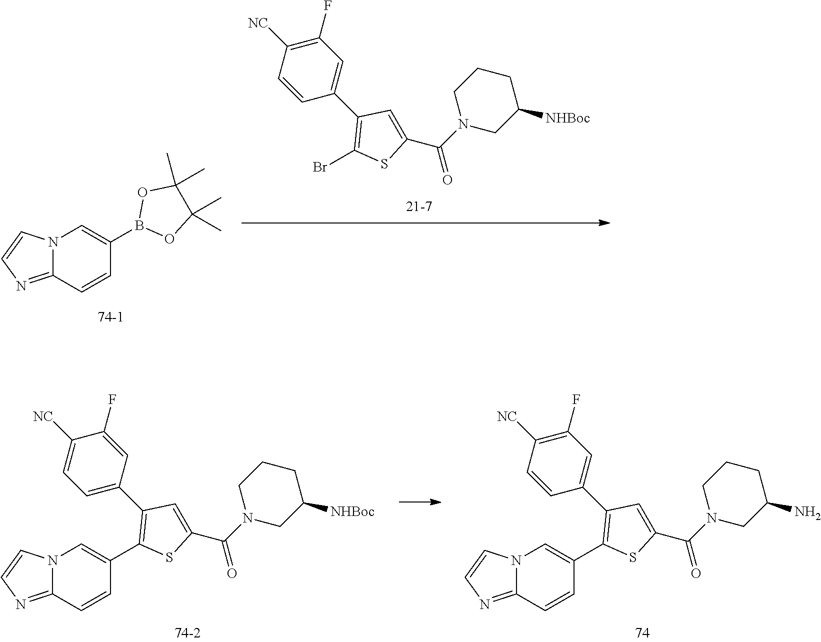 [SMILES: CC1(C)OB(c2ccc3nccn3c2)OC1(C)C ; CC(C)(C)OC(=O)N[C@@H]1CCCN(C(=O)c2cc(-c3ccc(C#N)c(F)c3)c(Br)s2)C1 ; CC(C)(C)OC(=O)N[C@@H]1CCCN(C(=O)c2cc(-c3ccc(C#N)c(F)c3)c(-c3ccc4nccn4c3)s2)C1 ; N#Cc1ccc(-c2cc(C(=O)N3CCC[C@@H](N)C3)sc2-c2ccc3nccn3c2)cc1F]

### Step 1

Under nitrogen atmosphere, compound 74-1 (150 mg, 615 μmol), compound 21-7 (331 mg, 615 μmol), potassium phosphate (261 mg, 1.23 mmol), and [1,1′-bis(di-tert-butylphosphino)ferrocene]dichloropalladium(II) (40.1 mg, 61.5 μmol) were dissolved in dioxane (4.00 mL) and water (0.800 mL), and the reaction mixture was stirred and reacted for 12 hours at 85° C. The reaction mixture was filtered through diatomite, and the filtrate was concentrated and purified by preparative silica gel thin layer chromatography (20/1, dichloromethane/methanol, R_{f}=0.24) to obtain compound 74-2. MS-ESI calculated for [M+H]⁺ 546, found 546.

### Step 2

Compound 74-2 (80 mg, 113 μmol) was dissolved in ethyl acetate (1.5 mL), and hydrochloride ethyl acetate solution (4 mol/L, 287 μL) was added thereto at 0° C., and the reaction mixture was stirred and reacted for 12 hours at 11° C. The reaction mixture was filtered. The solid was washed with ethyl acetate (0.400 mL), and the residue was purified by preparative high performance liquid chromatography (chromatographic column: Waters Xbridge 150*25 mm*5 μm; mobile phase: mobile phase A: ammonia aqueous solution with a volume fraction of 0.05%; mobile phase B: acetonitrile; B %: acetonitrile 17% to 47%, retention time of 9 minutes) to obtain compound 74. ¹H NMR (400 MHz, CD₃OD) δ=8.64-8.63 (m, 1H), 7.90-7.88 (m, 1H), 7.70-7.67 (m, 1H), 7.64-7.60 (m, 1H), 7.59 (s, 1H), 7.52 (d, J=9.6 Hz, 1H), 7.47-7.44 (m, 1H), 7.32-7.29 (m, 1H), 7.10-7.07 (m, 1H), 4.60-4.57 (m, 1H), 4.37-4.33 (m, 1H), 4.27-4.22 (m, 1H), 3.04-2.96 (m, 1H), 2.91-2.85 (m, 1H), 2.07-2.03 (m, 1H), 1.89-1.84 (m, 1H), 1.65-1.58 (m, 1H), 1.50-1.44 (m, 1H). MS-ESI calculated for [M+H]⁺ 446, found 446.

### Example 75

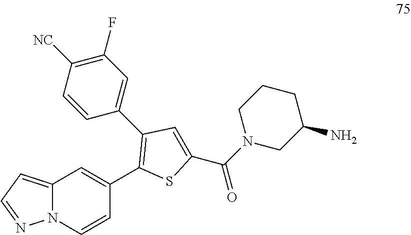 [SMILES: N#Cc1ccc(-c2cc(C(=O)N3CCC[C@@H](N)C3)sc2-c2ccn3nccc3c2)cc1F]

Synthetic Route:

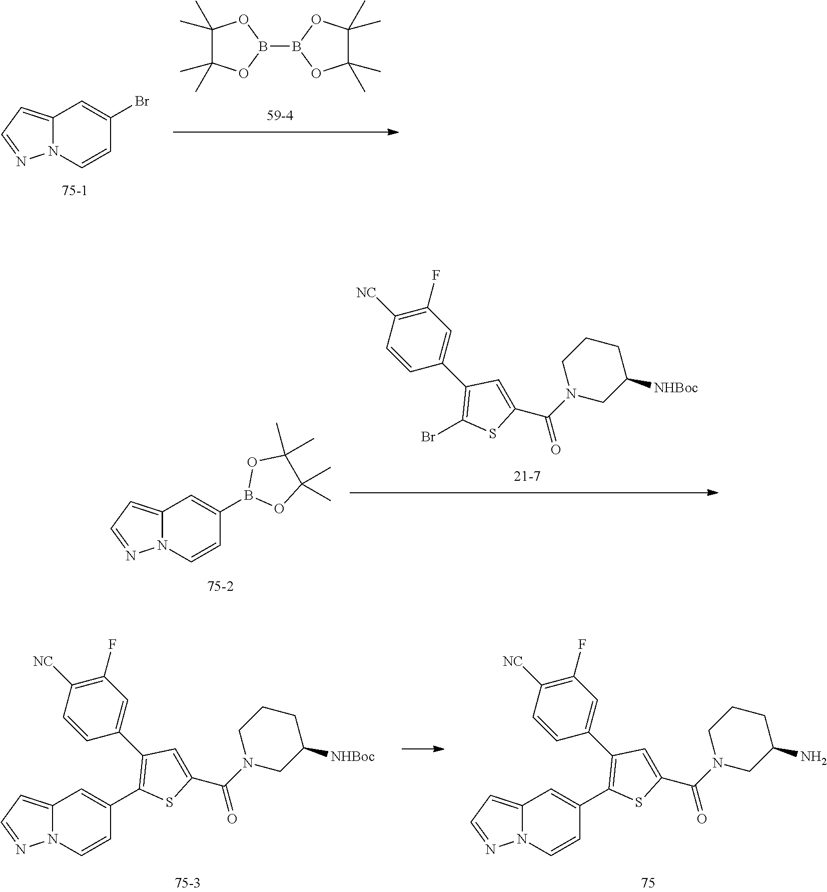

### Step 1

Under nitrogen atmosphere, compound 75-1 (116 mg, 589 μmol), compound 59-4 (179 mg, 706 μmol), potassium acetate (116 mg, 1.18 mmol), and [1,1′-bis(diphenylphosphino)ferrocene]dichloropalladium(II) dichloromethane (48.1 mg, 58.9 μmol) were dissolved in dioxane (4.00 mL). The reaction mixture was stirred and reacted for 12 hours at 85° C. The reaction mixture was filtered through diatomite, and the filtrate was concentrated to obtain crude compound 75-2, which was used directly in the next reaction step.

### Step 2

Under nitrogen atmosphere, compound 75-2 (144 mg, 590 μmol), compound 21-7 (317 mg, 589.93 μmol), potassium phosphate (250 mg, 1.18 mmol), and [1,1′-bis(di-tert-butylphosphino)ferrocene]dichloropalladium(II) (38.5 mg, 59.0 μmol) were dissolved in dioxane (8.00 mL) and water (1.60 mL), and the reaction mixture was stirred and reacted for 12 hours at 90° C. The reaction mixture was filtered through diatomite, and the filtrate was concentrated and purified by preparative silica gel thin layer chromatography (20/1, dichloromethane/methanol, R_{f}=0.27) to obtain compound 75-3. MS-ESI calculated for [M+H]⁺ 546, found 546.

### Step 3

Compound 75-3 (300 mg, 493 μmol) was dissolved in ethyl acetate (6.00 mL), and hydrochloride ethyl acetate solution (4 mol/L, 287 μL) was added thereto at 0° C., and the reaction mixture was stirred and reacted for 12 hours at 11° C. The reaction mixture was filtered, and the solid was washed with ethyl acetate (0.4 mL). The residue was purified by preparative high performance liquid chromatography (chromatographic column: Waters Xbridge 150*25 mm*5 μm; mobile phase: mobile phase A: ammonia aqueous solution with a volume fraction of 0.05%, mobile phase B: acetonitrile; B %: 25% to 55%, 9 minutes) to obtain compound 75. ¹H NMR (400 MHz, CDCl₃) δ=8.39-8.37 (m, 1H), 8.00-7.99 (m, 1H), 7.58-7.54 (m, 2H), 7.40 (s, 1H), 7.24-7.16 (m, 2H), 6.59-6.57 (m, 1H), 6.49-6.47 (m, 1H), 4.20-4.05 (m, 2H), 3.35-3.26 (m, 2H), 3.17-3.13 (m, 1H), 2.08-1.88 (m, 4H). MS-ESI calculated for [M+H]⁺ 446, found 446.

### Example 76

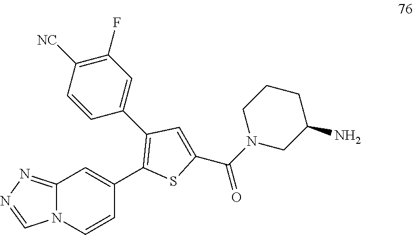 [SMILES: N#Cc1ccc(-c2cc(C(=O)N3CCC[C@@H](N)C3)sc2-c2ccn3cnnc3c2)cc1F]

Synthetic Route:

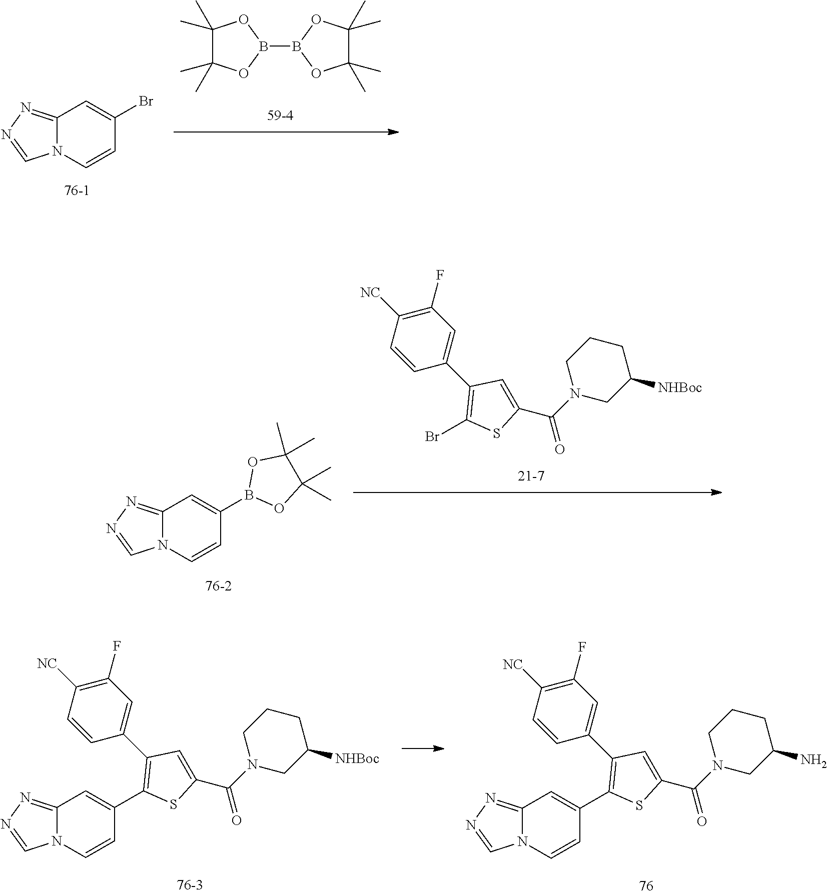

### Step 1

Under nitrogen atmosphere, compound 76-1 (500 mg, 2.52 mmol), compound 59-4 (769 mg, 3.03 mmol), potassium acetate (496 mg, 5.05 mmol), and [1,1′-bis(diphenylphosphino)ferrocene]dichloropalladium(II) dichloromethane (206 mg, 253 μmol) were dissolved in dioxane (16.0 mL). The reaction mixture was stirred and reacted for 12 hours at 80° C. The reaction mixture was filtered through diatomite, and the filtrate was concentrated to obtain crude compound 76-2, which was used directly in the next reaction step.

### Step 2

Under nitrogen atmosphere, compound 76-2 (620 mg, 2.53 mmol), compound 21-7 (1.36 g, 2.53 mmol), potassium phosphate (1.07 g, 5.06 mmol), and [1,1′-bis(di-tert-butylphosphino)ferrocene]dichloropalladium(II) (165 mg, 253 μmol) were dissolved in dioxane (16.0 mL) and water (3.20 mL), and the reaction mixture was stirred and reacted for 12 hours at 90° C. The reaction mixture was filtered through diatomite, and the filtrate was concentrated and purified by silica gel column chromatography (2/3/1, petroleum ether/ethyl acetate/ethanol) to obtain compound 76-3. MS-ESI calculated for [M+H]⁺ 547, found 547.

### Step 3

Compound 76-3 (1.30 g, 1.95 mmol) was dissolved in ethyl acetate (15.0 mL), and hydrochloride ethyl acetate solution (4 mol/L, 1.46 mL) was added thereto at 0° C., and the reaction mixture was stirred and reacted for 12 hours at 11° C. The reaction mixture was filtered. The solid was washed with ethyl acetate (1 mL), and the residue was purified by preparative high performance liquid chromatography (chromatographic column: 3_Phenomenex Luna C18 75*30 mm*3 μm; mobile phase: mobile phase A: hydrochloric acid aqueous solution with a volume fraction of 0.05%; mobile phase B: acetonitrile; B %: acetonitrile 13% to 23%, 6 minutes); the purified solution was adjusted to pH=9 to 10 with ammonia water, extracted with dichloromethane/methanol (10/1, 180 mL), dried over anhydrous sodium sulfate, filtered, and concentrated to obtain compound 76. ¹H NMR (400 MHz, DMSO-d₆) δ=9.28 (s, 1H), 8.50-8.48 (m, 1H), 7.93-7.86 (m, 2H), 7.70-7.66 (m, 2H), 7.35-7.33 (m, 1H), 6.69-6.67 (m, 1H), 4.16-4.05 (m, 2H), 3.12-3.02 (m, 2H), 2.77-2.67 (m, 1H), 1.89-1.85 (m, 1H), 1.77-1.66 (m, 3H), 1.50-1.42 (m, 1H), 1.32-1.23 (m, 1H). MS-ESI calculated for [M+H]⁺ 447, found 447.

### Example 77

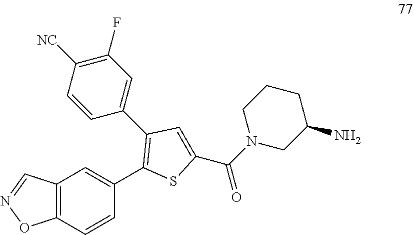 [SMILES: N#Cc1ccc(-c2cc(C(=O)N3CCC[C@@H](N)C3)sc2-c2ccc3oncc3c2)cc1F]

Synthetic Route:

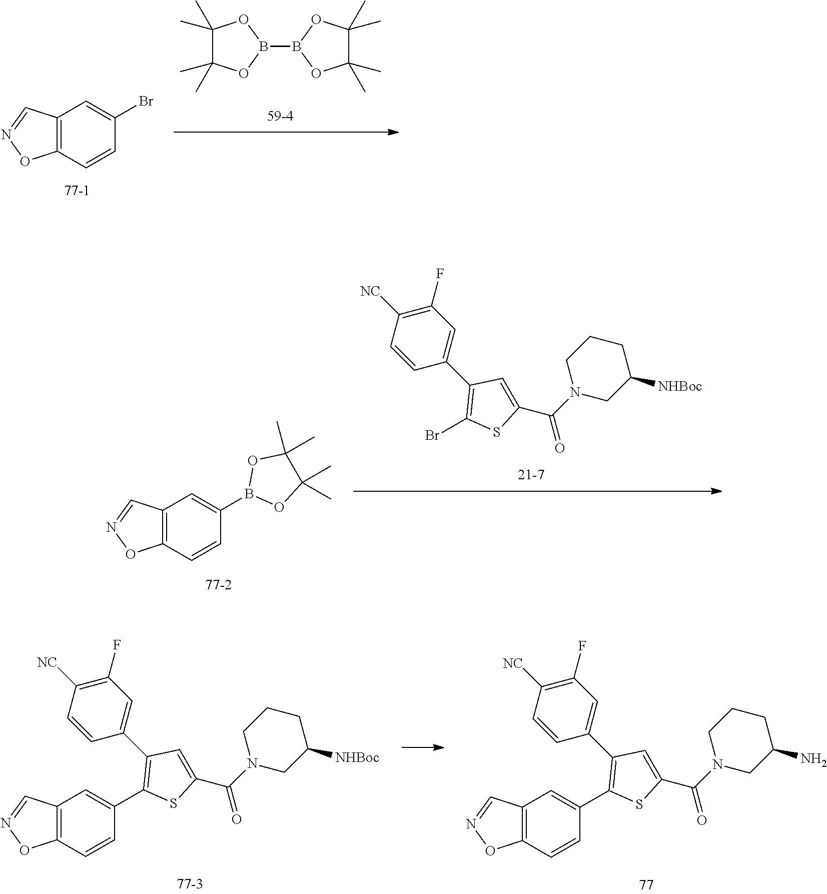

### Step 1

Under nitrogen atmosphere, compound 77-1 (117 mg, 591 μmol), compound 59-4 (180.05 mg, 709.03 μmol), potassium acetate (116 mg, 1.18 mmol), and [1,1′-bis(diphenylphosphino)ferrocene]dichloropalladium(II) dichloromethane (48.3 mg, 59.1 μmol) were dissolved in dioxane (4.00 mL). The reaction mixture was stirred and reacted for 12 hours at 85° C. The reaction mixture was filtered through diatomite, and the filtrate was concentrated to obtain crude compound 77-2, which was used directly in the next reaction step.

### Step 2

Under nitrogen atmosphere, compound 77-2 (145 mg, 592 μmol), compound 21-7 (318 mg, 592 μmol), potassium phosphate (251 mg, 1.18 mmol), and [1,1′-bis(di-tert-butylphosphino)ferrocene]dichloropalladium(II) (38.6 mg, 59.2 μmol) were dissolved in dioxane (8.00 mL) and water (1.60 mL), and the reaction mixture was stirred and reacted for 12 hours at 80° C. The reaction mixture was filtered through diatomite, and the filtrate was concentrated and purified by preparative silica gel thin layer chromatography (20/1, dichloromethane/methanol, R_{f}=0.26) to obtain compound 77-3. MS-ESI calculated for [M+H]⁺ 491, found 491.

### Step 3

Compound 77-3 (200 mg, 261 μmol) was dissolved in ethyl acetate (2.00 mL), and hydrochloride ethyl acetate solution (44 mol/L, 816 μL) was added thereto at 0° C., and the reaction mixture was stirred and reacted for 12 hours at 11° C. The reaction mixture was filtered, and the solid was washed with ethyl acetate (0.4 mL). The residue was purified by preparative high performance liquid chromatography (chromatographic column: 3_Phenomenex Luna C18 75*30 mm*3 μm; mobile phase: mobile phase A: hydrochloric acid aqueous solution with a volume fraction of 0.05%, mobile phase B: acetonitrile; B %: acetonitrile 23% to 33%, 6 minutes); the purified solution was adjusted to pH=9 to 10 with ammonia water, extracted with dichloromethane/methanol (10/1, 60 mL), dried over anhydrous sodium sulfate, filtrated, and concentrated to obtain compound 77. ¹H NMR (400 MHz, CD₃OD) δ=7.71-7.67 (m, 1H), 7.51 (s, 1H), 7.40 (d, J=2.0 Hz, 1H), 7.35-7.33 (m, 1H), 7.28-7.20 (m, 2H), 6.78 (d, J=8.4 Hz, 1H), 4.62-4.59 (m, 1H), 4.37-4.33 (m, 1H), 4.20-4.16 (m, 1H), 3.27-3.21 (m, 1H), 3.17-3.14 (m, 1H), 2.15-2.12 (m, 1H), 1.78-1.86 (m, 1H), 1.69-1.60 (m, 2H). MS-ESI calculated for [M+H]⁺ 447, found 447.

### Example 78

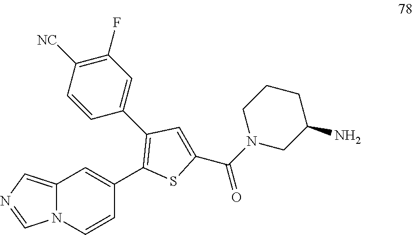 [SMILES: N#Cc1ccc(-c2cc(C(=O)N3CCC[C@@H](N)C3)sc2-c2ccn3cncc3c2)cc1F]

Synthetic Route:

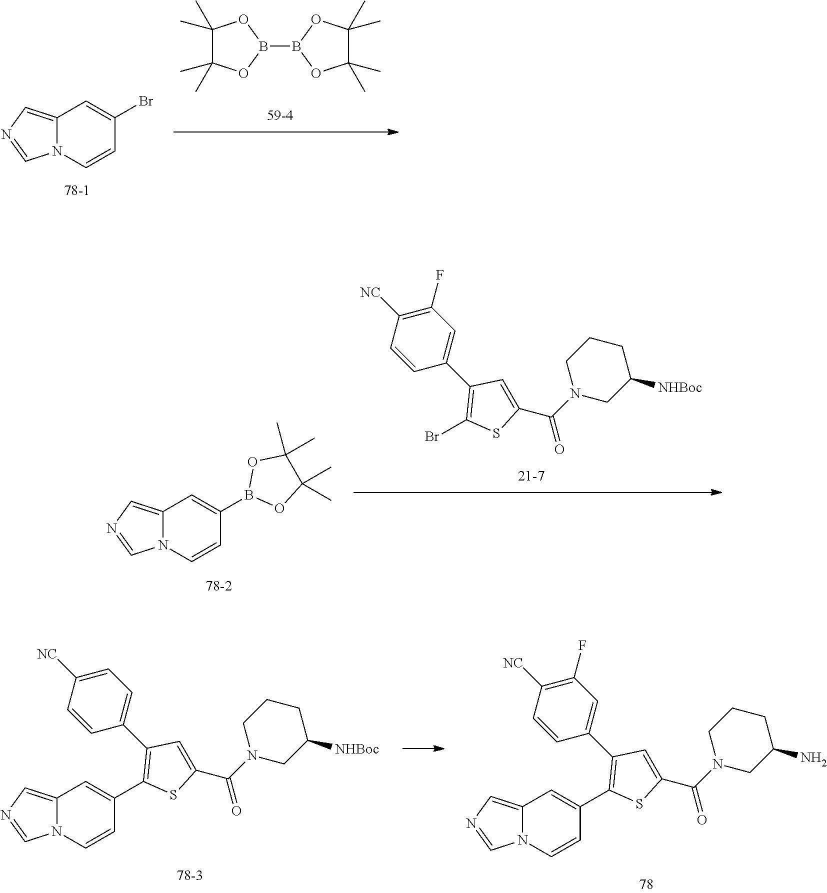

### Step 1

Under nitrogen atmosphere, compound 78-1 (130 mg, 660 μmol), compound 59-4 (201 mg, 792 μmol), potassium acetate (130 mg, 1.32 mmol), and [1,1′-bis(diphenylphosphino)ferrocene]dichloropalladium(II) dichloromethane (53.9 mg, 66.0 μmol) were dissolved in dioxane (4.00 mL). The reaction mixture was stirred and reacted for 12 hours at 85° C. The reaction mixture was filtered through diatomite, and the filtrate was concentrated to obtain crude compound 78-2, which was used directly in the next reaction step.

### Step 2

Under nitrogen atmosphere, compound 78-2 (160 mg, 655 μmol), compound 21-7 (353 mg, 655 μmol), potassium phosphate (278 mg, 1.31 mmol), and [1,1′-bis(di-tert-butylphosphino)ferrocene]dichloropalladium(II) (42.7 mg, 65.6 μmol) were dissolved in dioxane (6.00 mL) and water (1.60 mL), and the reaction mixture was stirred and reacted for 12 hours at 85° C. The reaction mixture was filtered through diatomite, and the filtrate was concentrated and purified by preparative silica gel thin layer chromatography (20/1, dichloromethane/methanol, R_{f}=0.30) to obtain compound 78-3. MS-ESI calculated for [M+H]⁺ 546, found 546.

### Step 3

Compound 78-3 (200 mg, 367 μmol) was dissolved in ethyl acetate (4.00 mL), and hydrochloride ethyl acetate solution (4 mol/L, 367 μL) was added thereto at 0° C., and the reaction mixture was stirred and reacted for 12 hours at 11° C. The reaction mixture was filtered, and the solid was washed with ethyl acetate (0.4 mL). The residue was purified by preparative high performance liquid chromatography (chromatographic column: 3_Phenomenex Luna C18 75*30 mm*3 μm; mobile phase: mobile phase A: hydrochloric acid aqueous solution with a volume fraction of 0.05%, mobile phase B: acetonitrile; B %: 5% to 25%, 6 minutes); the purified solution was adjusted to pH=9 to 10 with ammonia water, extracted with dichloromethane/methanol (10/1, 90 mL), dried over anhydrous sodium sulfate, filtrated, and concentrated to obtain compound 78. ¹H NMR (400 MHz, CD₃OD) δ=8.34 (s, 1H), 8.17 (d, J=7.6 Hz, 1H), 7.74-7.67 (m, 2H), 7.55 (s, 1H), 7.47-7.44 (m, 2H), 7.36-7.34 (m, 1H), 6.42-6.40 (m, 1H), 4.36-4.33 (m, 1H), 4.28-4.20 (m, 1H), 3.24-3.14 (m, 1H), 3.05-2.95 (m, 1H), 2.91-2.85 (m, 1H), 2.08-2.04 (m, 1H), 1.89-1.83 (m, 1H), 1.67-1.56 (m, 1H), 1.50-1.40 (m, 1H). MS-ESI calculated for [M+H]⁺ 446, found 446.

### Example 79

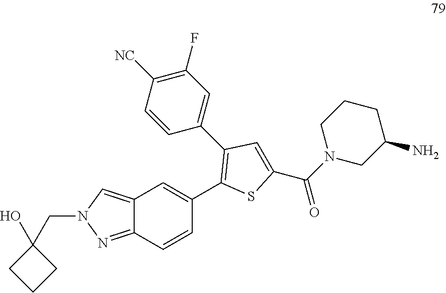 [SMILES: N#Cc1ccc(-c2cc(C(=O)N3CCC[C@@H](N)C3)sc2-c2ccc3nn(CC4(O)CCC4)cc3c2)cc1F]

Synthetic Route:

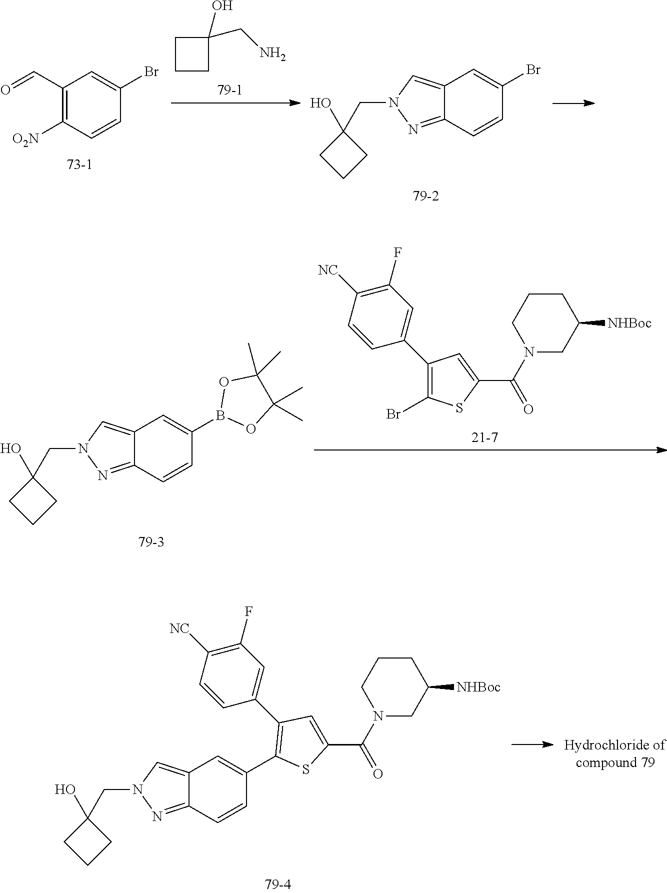

### Step 1

Compound 73-1 (3.79 g, 16.5 mmol) was dissolved in isopropanol (20.0 mL), then 79-1 (2.00 g, 19.8 mmol) was added thereto, and the reaction mixture was stirred at 80° C. for 4 hours, then cooled to 25° C., added dropwise with tributylphosphine (12.2 mL, 49.4 mmol), and stirred and reacted for 12 hours at 80° C. The reaction mixture was added with water (80 mL), and extracted with ethyl acetate (80 mL×2). The organic phase was washed with saturated brine (80 mL×1), dried over anhydrous sodium sulfate, filtered, and concentrated under reduced pressure. The residue was purified by silica gel column chromatography (3/1, petroleum ether/ethyl acetate, R_{f}=0.22) to obtain compound 79-2. ¹H NMR (400 MHz, CD₃OD) δ=8.23 (s, 1H), 7.89 (d, J=1.2 Hz, 1H), 7.54 (d, J=9.2 Hz, 1H), 7.34 (dd, J=1.6, 9.2 Hz, 1H), 4.53 (s, 2H), 2.25-2.14 (m, 2H), 2.12-1.98 (m, 2H), 1.85-1.74 (m, 1H), 1.72-1.61 (m, 1H). MS-ESI calculated for [M+H]⁺ 281, found 281.

### Step 2

Under nitrogen atmosphere, compound 79-2 (1.60 g, 5.69 mmol), bis(pinacolato)diboron (4.34 g, 17.1 mmol), and potassium acetate (1.68 g, 17.1 mmol) were dissolved in dioxane (30.0 mL), and the reaction mixture was added with [1,1′-bis(diphenylphosphino)ferrocene]dichloropalladium(II) (833 mg, 1.14 mmol). The reaction mixture was stirred and reacted at 100° C. for 12 hours. The reaction mixture was concentrated under reduced pressure, and the residue was purified by silica gel column chromatography (1/1, petroleum ether/ethyl acetate, R_{f}=0.29) to obtain compound 79-3. ¹H NMR (400 MHz, CD₃OD) δ=8.30 (s, 1H), 8.21 (s, 1H), 7.63-7.52 (m, 2H), 4.53 (s, 2H), 2.24-2.14 (m, 2H), 2.10-2.02 (m, 2H), 1.87-1.75 (m, 1H), 1.72-1.62 (m, 1H), 1.35 (s, 12H). MS-ESI calculated for [M+H]⁺ 329, found 329.

### Step 3

Under nitrogen atmosphere, compound 21-7 (697 mg, 1.37 mmol), compound 79-3 (900 mg, 2.74 mmol), and sodium carbonate (436 mg, 4.11 mmol) were dissolved in dioxane (24.0 mL) and water (6.00 mL), and the reaction mixture was added with [1,1′-bis(diphenylphosphino)ferrocene]dichloropalladium(II) (100 mg, 137 μmol), and the reaction mixture was stirred and reacted for 12 hours at 100° C. The reaction mixture was concentrated under reduced pressure, and the residue was purified by silica gel column chromatography (1/1, petroleum ether/ethyl acetate, R_{f}=0.22) to obtain compound 79-4. MS-ESI calculated for [M+H]⁺ 630, found 630.

### Step 4

Compound 79-4 (300 mg, 476 μmol) was dissolved in ethyl acetate (15.0 mL), and hydrochloride ethyl acetate solution (4 mol/L, 1.19 mL) was added thereto, and the reaction mixture was stirred and reacted for 3 hours at 25° C. After the reaction mixture was concentrated, the residue was purified by preparative high performance liquid chromatography (chromatographic column: Phenomenex Genimi NX C18 150*40 mm*5 μm, mobile phase A: hydrochloric acid aqueous solution with a volume fraction of 0.05%; mobile phase B: acetonitrile; B %: 5% to 35%, 10 minutes) to obtain the hydrochloride of compound 79. ¹H NMR (400 MHz, CD₃OD) δ=8.63 (s, 1H), 7.94 (s, 1H), 7.75-7.57 (m, 3H), 7.43-7.22 (m, 3H), 4.66 (s, 2H), 4.47-4.37 (m, 1H), 4.23-4.10 (m, 1H), 3.61-3.39 (m, 3H), 2.29-2.16 (m, 3H), 2.15-2.02 (m, 2H), 1.98-1.67 (m, 5H). MS-ESI calculated for [M+H]⁺ 530, found 530.

### Example 80

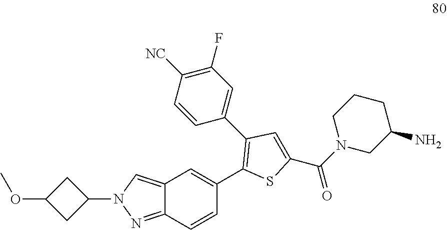 [SMILES: COC1CC(n2cc3cc(-c4sc(C(=O)N5CCC[C@@H](N)C5)cc4-c4ccc(C#N)c(F)c4)ccc3n2)C1]

Synthetic Route:

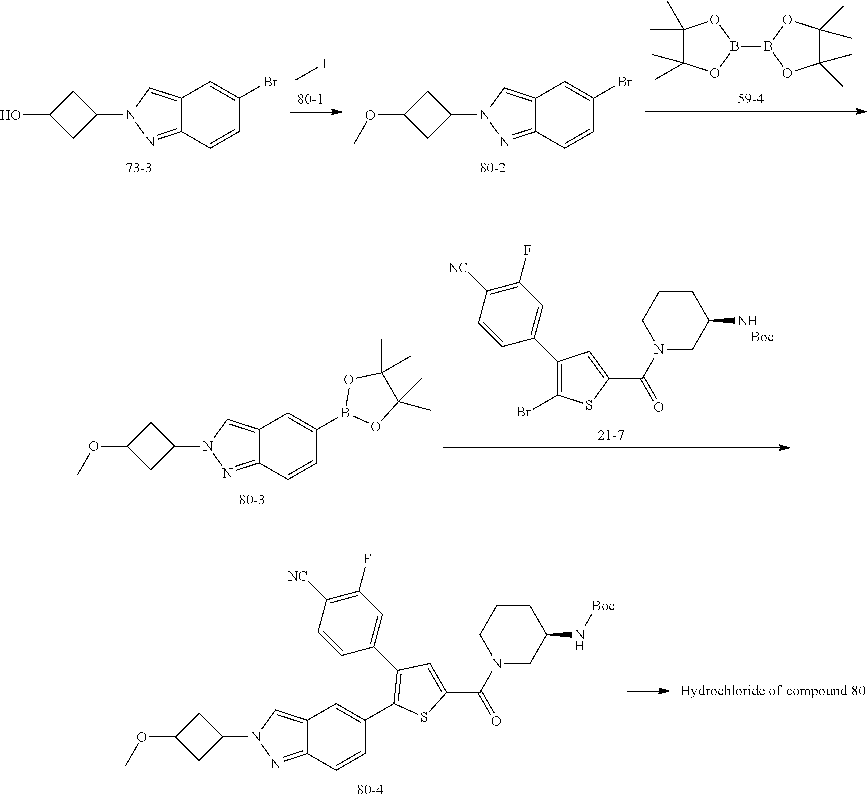

### Step 1

Under nitrogen atmosphere, compound 73-3 (1.00 g, 3.74 mmol) was dissolved in anhydrous dichloromethane (10.0 mL), and iodomethane (5.31 g, 37.4 mmol) and silver oxide (1.74 g, 7.49 mmol) were added thereto. The reaction mixture was stirred for 12 hours at 25° C., and concentrated under reduced pressure. The residue was purified by silica gel column chromatography (1/1, petroleum ether/ethyl acetate, R_{f}=0.42, 0.51) to obtain compound 80-2. MS-ESI calculated for [M+H]⁺ 281, found 281.

### Step 2

Under nitrogen atmosphere, compound 80-2 (100 mg, 356 μmol), bis(pinacolato)diboron (271 mg, 1.07 mmol), and potassium acetate (175 mg, 1.78 mmol) were dissolved in dioxane (10.0 mL), and the reaction mixture was added with [1,1′-bis(diphenylphosphino)ferrocene]dichloropalladium(II) (52.1 mg, 71.1 μmol), and the reaction mixture was stirred and reacted for 12 hours at 95° C., and concentrated under reduced pressure. The residue was purified by silica gel column chromatography (1/1, petroleum ether/ethyl acetate, R_{f}=0.60) to obtain compound 80-3. MS-ESI calculated for [M+H]⁺ 329, found 329.

### Step 3

Under nitrogen atmosphere, compound 21-7 (150 mg, 295 μmol), compound 80-3 (107 mg, 325 μmol), and sodium carbonate (93.8 mg, 885 μmol) were dissolved in dioxane (8.00 mL) and water (2.00 mL), and the reaction mixture was added with [1,1′-bis(di-tert-butylphosphino)ferrocene]dichloropalladium(II) (19.2 mg, 29.5 μmol). The reaction mixture was stirred and reacted for 2 hours at 80° C., added with water (20 mL), and extracted with ethyl acetate (20 mL×1). The organic phase was washed with saturated brine (20 mL×1), dried over anhydrous sodium sulfate, filtered, and concentrated under reduced pressure, and the residue was purified by silica gel column chromatography (1/1, petroleum ether/ethyl acetate, R_{f}=0.21) to obtain compound 80-4. MS-ESI calculated for [M+H]⁺ 630, found 630.

### Step 4

Under nitrogen atmosphere, compound 80-4 (130 mg, 0.206 mmol) was dissolved in anhydrous dichloromethane (5.00 mL), and trimethylsilyl trifluoromethanesulfonate (91.8 mg, 0.413 mmol) was added dropwise at 0° C. The reaction mixture was stirred for 1 hour at 25° C., added with water (1 mL), concentrated under reduced pressure, and then separated by preparative high performance liquid chromatography (chromatographic column: Phenomenex Genimi NX C18 150*40 mm*5 μm; mobile phase: mobile phase A: hydrochloric acid aqueous solution with a volume fraction of 0.05%; mobile phase B: acetonitrile; B %: 15 to 45%, 10 minutes) to obtain the hydrochloride of compound 80. ¹H NMR (400 MHz, CD₃OD) δ=8.72-8.61 (m, 1H), 7.87 (s, 1H), 7.70-7.58 (m, 3H), 7.37-7.21 (m, 3H), 5.40-5.32 (m, 0.3H), 4.91-4.85 (m, 0.7H), 4.54-4.40 (m, 1H), 4.31-4.26 (m, 0.3H), 4.24-4.12 (m, 1H), 3.98-3.89 (m, 0.7H), 3.58-3.40 (m, 3H), 3.34-3.32 (m, 3H), 3.09-3.01 (m, 1.4H), 2.95-2.87 (m, 0.6H), 2.76-2.68 (m, 0.6H), 2.65-2.55 (m, 1.4H), 2.30-2.16 (m, 1H), 2.00-1.88 (m, 1H), 1.87-1.69 (m, 2H). MS-ESI calculated for [M+H]⁺ 530, found 530.

### Example 81

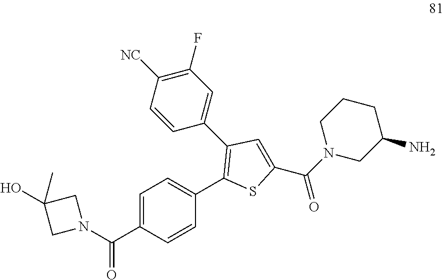 [SMILES: CC1(O)CN(C(=O)c2ccc(-c3sc(C(=O)N4CCC[C@@H](N)C4)cc3-c3ccc(C#N)c(F)c3)cc2)C1]

Synthetic Route:

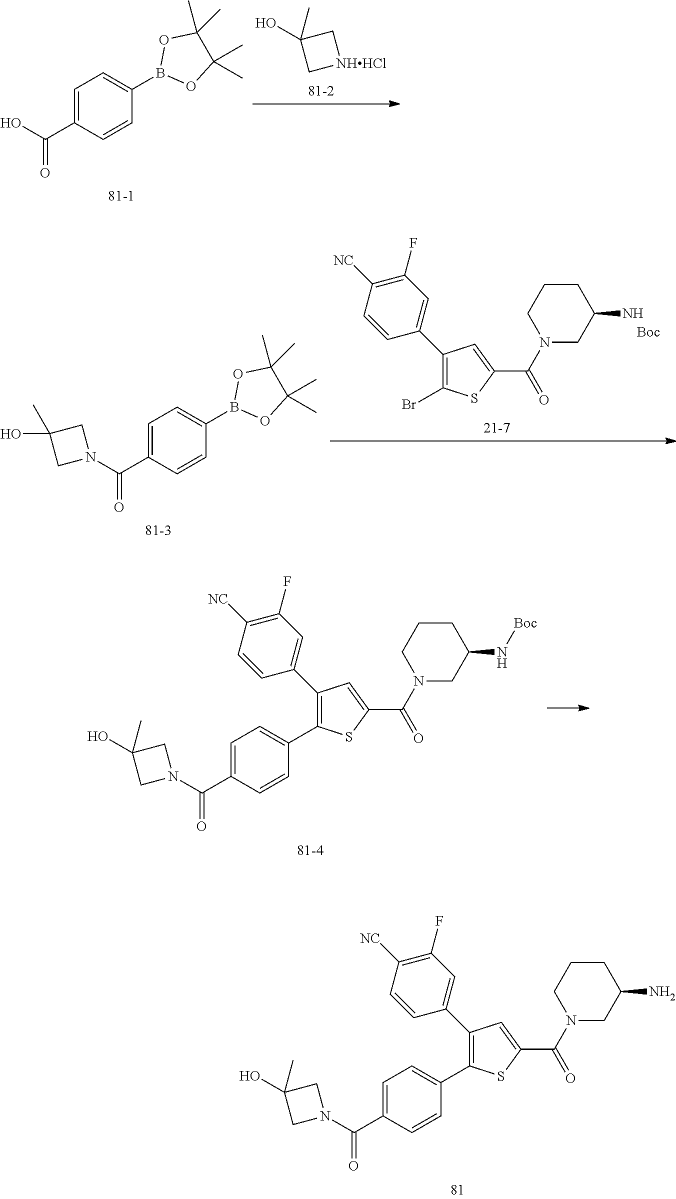

### Step 1

Under nitrogen atmosphere, compound 81-1 (200 mg, 0.806 mmol) was dissolved in anhydrous dichloromethane (10.0 mL), and then triethylamine (326 mg, 3.22 mmol), 81-2 (130 mg, 1.05 mmol), and 2-(7-azabenzotriazol-1-yl)-N,N,N′,N′-tetramethyluronium hexafluorophosphate (399 mg, 1.05 mmol) were added thereto. The reaction mixture was stirred for 2 hours at 25° C. The reaction mixture was concentrated under reduced pressure, and the residue was purified by silica gel column chromatography (1/1, petroleum ether/ethyl acetate, R_{f}=0.26) to obtain compound 81-3. MS-ESI calculated for [M+H]⁺ 318, found 318.

### Step 2

Under nitrogen atmosphere, compound 21-7 (260 mg, 0.511 mmol), compound 81-3 (243 mg, 0.767 mmol), and sodium carbonate (163 mg, 1.53 mmol) were dissolved in dioxane (8.00 mL) and water (2.00 mL), and the reaction mixture was added with [1,1′-bis(di-tert-butylphosphino)ferrocene]dichloropalladium(II) (33.3 mg, 51.1 μmol). The reaction mixture was stirred and reacted for 2 hours at 80° C., added with water (20 mL), and extracted with ethyl acetate (20 mL×1). The organic phase was washed with saturated brine (20 mL×1), dried over anhydrous sodium sulfate, filtered, and concentrated under reduced pressure. The residue was purified by silica gel column chromatography (1/1, petroleum ether/ethyl acetate, R_{f}=0.21) to obtain compound 81-4. MS-ESI calculated for [M+H]⁺ 619, found 619.

### Step 3

Compound 81-4 (200 mg, 0.323 mmol) was dissolved in ethyl acetate (5.00 mL), and hydrochloride ethyl acetate solution (4 mol/L, 5.00 mL) was added dropwise thereto at 25° C. The reaction mixture was stirred for 1 hour at 25° C., concentrated under reduced pressure, and separated by preparative high performance liquid chromatography (chromatographic column: Phenomenex Genimi NX 80*40 mm*3 μm; mobile phase: mobile phase A: ammonia aqueous solution with a volume fraction of 0.05%; mobile phase B: acetonitrile; B %: 29 to 59%, 8 minutes) to obtain compound 81. ¹H NMR (400 MHz, CD₃OD) δ=7.71-7.62 (m, 3H), 7.54 (s, 1H), 7.43-7.37 (m, 2H), 7.32 (d, J=11.2 Hz, 1H), 7.24 (d, J=8.0 Hz, 1H), 4.40-4.30 (m, 1H), 4.28-4.17 (m, 3H), 4.10-4.00 (m, 2H), 3.29-3.09 (m, 1H), 3.08-2.92 (m, 1H), 2.91-2.80 (m, 1H), 2.11-2.01 (m, 1H), 1.90-1.80 (m, 1H), 1.67-1.55 (m, 1H), 1.50 (s, 3H), 1.47-1.38 (m, 1H). MS-ESI calculated for [M+H]⁺ 519, found 519.

### Example 82

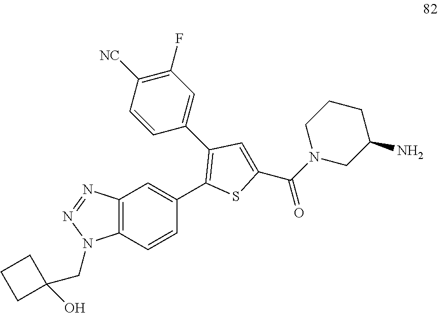 [SMILES: N#Cc1ccc(-c2cc(C(=O)N3CCC[C@@H](N)C3)sc2-c2ccc3c(c2)nnn3CC2(O)CCC2)cc1F]

Synthetic Route:

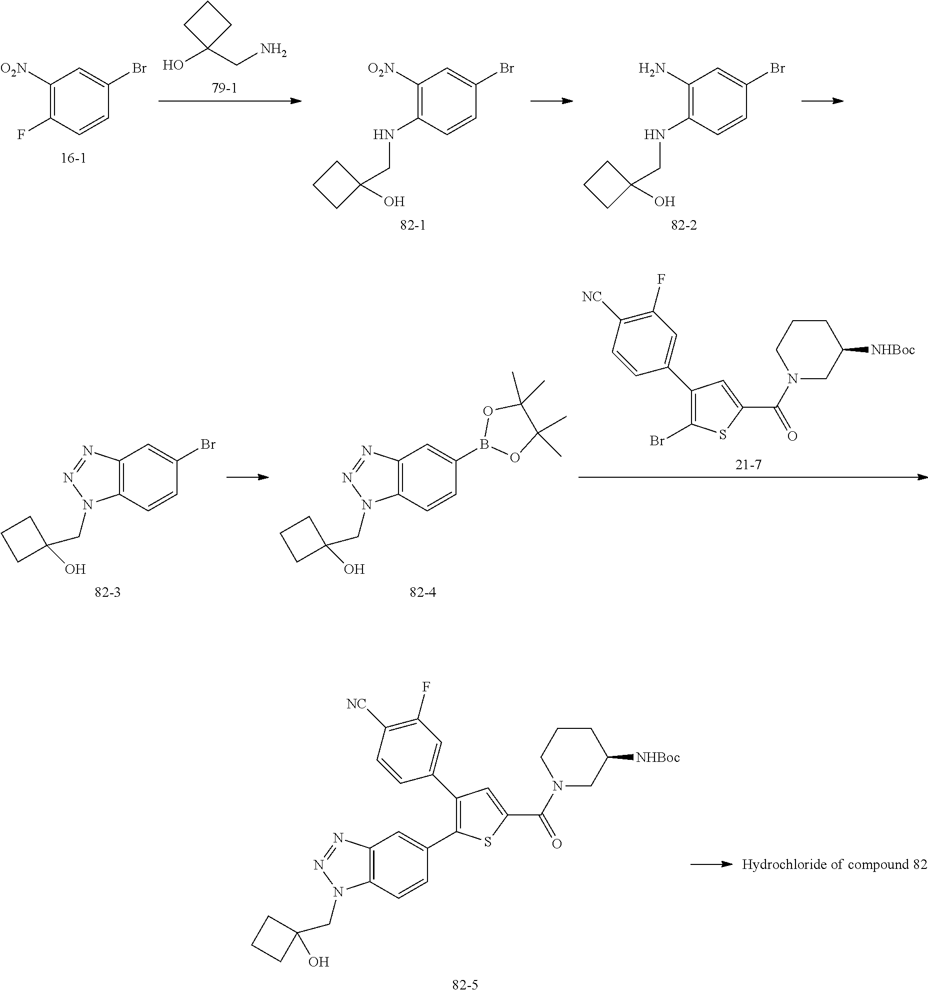

### Step 1

Compounds 16-1 (9.06 g, 41.2 mmol) and 79-1 (5.00 g, 49.4 mmol) were dissolved in N,N-dimethylformamide (40.0 mL), and N,N-diisopropylethylamine (10.7 g, 82.4 mmol) was added thereto, and the reaction mixture was stirred and reacted for 12 hours at 80° C. The reaction mixture was added with water (150 mL), and extracted with ethyl acetate (150 mL×2). The organic phase was washed with saturated brine (150 mL×1), dried over anhydrous sodium sulfate, filtered, and concentrated under reduced pressure. The residue was purified by silica gel column chromatography (1/1, petroleum ether/ethyl acetate, R_{f}=0.48) to obtain compound 82-1. ¹H NMR (400 MHz, CD₃OD) δ=8.20 (d, J=2.4 Hz, 1H), 7.54 (dd, J=2.4, 9.2 Hz, 1H), 7.03 (d, J=9.2 Hz, 1H), 3.44 (s, 2H), 2.22-2.08 (m, 4H), 1.89-1.75 (m, 1H), 1.73-1.58 (m, 1H). MS-ESI calculated for [M+H]⁺ 301, found 301.

### Step 2

Compound 82-1 (3.00 g, 9.96 mmol) was dissolved in concentrated hydrochloric acid (20.0 mL), then tin dichloride (6.74 g, 29.9 mmol) was added thereto, and the reaction mixture was stirred and reacted for 1 hour at 25° C. The reaction mixture was adjusted to pH=13 with sodium hydroxide (1 mol/L aqueous solution), and extracted with ethyl acetate (150 mL×2). The organic phase was washed with saturated brine (150 mL×1), dried over anhydrous sodium sulfate, filtered, and concentrated under reduced pressure. The residue was purified by silica gel column chromatography (1/1, petroleum ether/ethyl acetate, R_{f}=0.21) to obtain compound 82-2. ¹H NMR (400 MHz, CD₃OD) δ=6.83 (d, J=2.4 Hz, 1H), 6.77 (dd, J=2.4, 8.4 Hz, 1H), 6.54 (d, J=8.4 Hz, 1H), 3.17 (s, 2H), 2.23-2.04 (m, 4H), 1.87-1.74 (m, 1H), 1.69-1.55 (m, 1H). MS-ESI calculated for [M+H]⁺ 271, found 271.

### Step 3

Compound 82-2 (2.30 g, 8.48 mmol) was dissolved in acetic acid (20.0 mL), and a solution of sodium nitrite (878 mg, 12.7 mmol) in water (10.0 mL) was added dropwise thereto, and the reaction mixture was stirred for 4 hours at 25° C. The reaction mixture was added with water (50 mL), and extracted with ethyl acetate (50 mL×2). The organic phase was washed with saturated ammonium chloride aqueous solution (50 mL), dried over anhydrous sodium sulfate, filtered, and concentrated under reduced pressure. The residue was purified by silica gel column chromatography (1/1, petroleum ether/ethyl acetate, R_{f}=0.59) to obtain compound 82-3. ¹H NMR (400 MHz, CD₃OD) δ=8.16 (d, J=1.6 Hz, 1H), 7.81 (d, J=8.8 Hz, 1H), 7.64 (dd, J=1.6, 8.8 Hz, 1H), 4.82 (s, 2H), 2.35-2.29 (m, 2H), 2.15-2.01 (m, 2H), 1.92-1.71 (m, 2H). MS-ESI calculated for [M+H]⁺ 282, found 282.

### Step 4

Under nitrogen atmosphere, compound 82-3 (0.90 g, 3.19 mmol), bis(pinacolato)diboron (2.43 g, 9.57 mmol), and potassium acetate (939 mg, 9.57 mmol) were dissolved in dioxane (20.0 mL), and the reaction mixture was added with [1,1′-bis(diphenylphosphino)ferrocene]dichloropalladium(II) (233 mg, 319 μmol). The reaction mixture was stirred and reacted at 100° C. for 12 hours. The reaction mixture was concentrated under reduced pressure, and the residue was purified by silica gel column chromatography (1/1, petroleum ether/ethyl acetate, R_{f}=0.37) to obtain compound 82-4. ¹H NMR (400 MHz, CD₃OD) δ=8.35 (s, 1H), 7.87-7.78 (m, 2H), 4.80 (s, 2H), 2.35-2.28 (m, 2H), 2.11-2.01 (m, 2H), 1.89-1.68 (m, 2H), 1.38 (s, 12H). MS-ESI calculated for [M+H]⁺ 330, found 330.

### Step 5

Under nitrogen atmosphere, compound 21-7 (321 mg, 632 μmol), compound 82-4 (250 mg, 759 μmol), and sodium carbonate (201 mg, 1.90 mmol) were dissolved in dioxane (16.0 mL) and water (4.00 mL), and the reaction mixture was added with [1,1′-bis(diphenylphosphino)ferrocene]dichloropalladium(II) (46.3 mg, 63.3 μmol), and the reaction mixture was stirred and reacted for 12 hours at 100° C. The reaction mixture was concentrated under reduced pressure, and the residue was purified by silica gel column chromatography (1/1, petroleum ether/ethyl acetate, R_{f}=0.35) to obtain compound 82-5. MS-ESI calculated for [M+H]⁺ 631, found 631.

### Step 6

Compound 82-5 (370 mg, 587 μmol) was dissolved in ethyl acetate (10.0 mL), and hydrochloride ethyl acetate solution (4 mol/L, 1.47 mL) was added thereto, and the reaction mixture was stirred and reacted for 3 hours at 25° C. After the reaction mixture was concentrated, the residue was purified by preparative high performance liquid chromatography (chromatographic column: Phenomenex Genimi NX C18 150*40 mm*5 μm, mobile phase A: hydrochloric acid aqueous solution with a volume fraction of 0.05%; mobile phase B: acetonitrile; B %: 10% to 40%, 10 minutes) to obtain the hydrochloride of compound 82. ¹H NMR (400 MHz, CD₃OD) δ=7.97 (s, 1H), 7.87 (d, J=8.4 Hz, 1H), 7.70-7.63 (m, 2H), 7.46-7.34 (m, 2H), 7.27 (d, J=7.6 Hz, 1H), 4.85 (s, 2H), 4.46-4.40 (m, 1H), 4.21-4.17 (m, 1H), 3.63-3.40 (m, 3H), 2.38-2.30 (m, 2H), 2.25-2.21 (m, 1H), 2.15-2.03 (m, 2H), 1.98-1.90 (m, 1H), 1.89-1.71 (m, 4H). MS-ESI calculated for [M+H]⁺ 531, found 531.

### Example 83

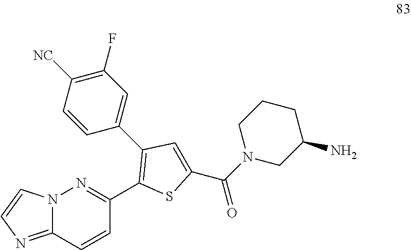 [SMILES: N#Cc1ccc(-c2cc(C(=O)N3CCC[C@@H](N)C3)sc2-c2ccc3nccn3n2)cc1F]

Synthetic Route:

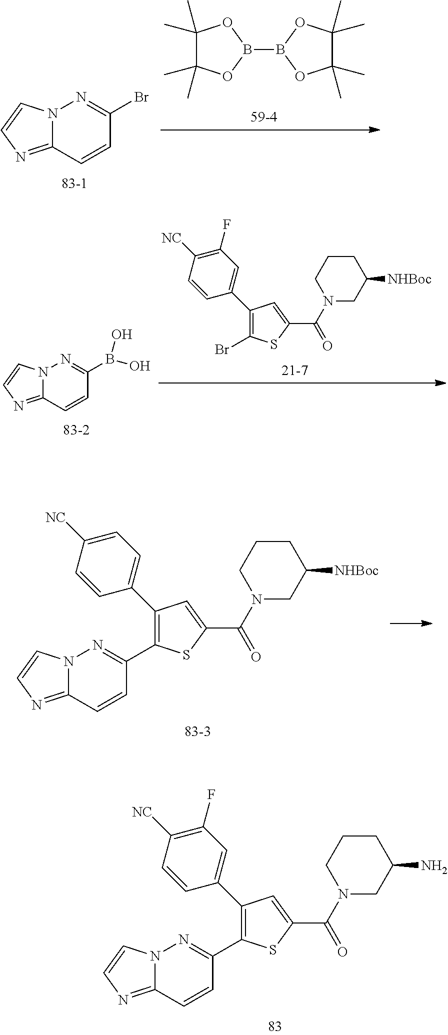

### Step 1

Under nitrogen atmosphere, compound 83-1 (200 mg, 1.01 mmol), compound 59-4 (308 mg, 1.21 mmol), potassium acetate (198 mg, 2.02 mmol), and [1,1′-bis(diphenylphosphino)ferrocene]dichloropalladium(II) dichloromethane (82.5 mg, 101 μmol) were dissolved in dioxane (7.00 mL). The reaction mixture was stirred and reacted for 12 hours at 90° C. The reaction mixture was filtered, and the solid was washed with dichloromethane (3 mL) and dried to obtain crude compound 83-2, which was used directly in the next reaction step.

### Step 2

Under nitrogen atmosphere, compound 83-2 (170 mg, 1.04 mmol), compound 21-7 (561 mg, 1.04 mmol), potassium phosphate (443 mg, 2.09 mmol), and [1,1′-bis(di-tert-butylphosphino)ferrocene]dichloropalladium(II) (68.0 mg, 104 μmol) were dissolved in dioxane (10.0 mL) and water (2.00 mL), and the reaction mixture was stirred and reacted for 12 hours at 90° C. The reaction mixture was filtered through diatomite, and the filtrate was concentrated and purified by preparative silica gel thin layer chromatography (20/1, dichloromethane/methanol, R_{f}=0.27) to obtain compound 83-3. MS-ESI calculated for [M+H]⁺ 547, found 547.

### Step 3

Compound 83-3 (120 mg, 203 μmol) was dissolved in ethyl acetate (2.00 mL), and hydrochloride ethyl acetate solution (4 mol/L, 203 μL) was added thereto at 0° C., and the reaction mixture was stirred and reacted for 12 hours at 11° C. The reaction mixture was filtered, and the solid was washed with ethyl acetate (0.4 mL). The residue was purified by preparative high performance liquid chromatography (chromatographic column: Waters Xbridge 150*25 mm*5 μm; mobile phase: mobile phase A: ammonia aqueous solution with a volume fraction of 0.05%, mobile phase B: acetonitrile; B %: acetonitrile 18% to 48%, 9 minutes) to obtain compound 83. ¹H NMR (400 MHz, CD₃OD) δ=8.17 (s, 1H), 7.90 (d, J=9.6 Hz, 1H), 7.80-7.75 (m, 2H), 7.56-7.54 (m, 2H), 7.39-7.36 (s, 1H), 6.99 (d, J=9.6 Hz, 1H), 4.33-4.23 (m, 2H), 3.14-2.99 (m, 2H), 2.90-2.84 (m, 1H), 2.08-2.04 (m, 1H), 1.89-1.84 (m, 1H), 1.68-1.58 (m, 1H), 1.50-1.44 (m, 1H). MS-ESI calculated for [M+H]⁺ 447, found 447.

### Example 84

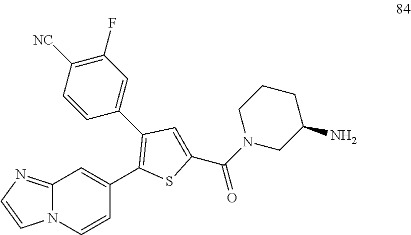 [SMILES: N#Cc1ccc(-c2cc(C(=O)N3CCC[C@@H](N)C3)sc2-c2ccn3ccnc3c2)cc1F]

Synthetic Route:

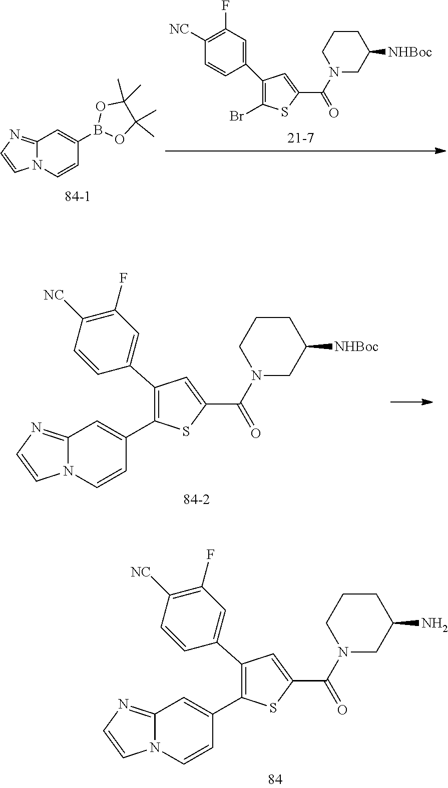 [SMILES: CC1(C)OB(c2ccn3ccnc3c2)OC1(C)C ; CC(C)(C)OC(=O)N[C@@H]1CCCN(C(=O)c2cc(-c3ccc(C#N)c(F)c3)c(Br)s2)C1 ; CC(C)(C)OC(=O)N[C@@H]1CCCN(C(=O)c2cc(-c3ccc(C#N)c(F)c3)c(-c3ccn4ccnc4c3)s2)C1 ; N#Cc1ccc(-c2cc(C(=O)N3CCC[C@@H](N)C3)sc2-c2ccn3ccnc3c2)cc1F]

### Step 1

Under nitrogen atmosphere, compound 84-1 (111 mg, 454 μmol), compound 21-7 (244 mg, 454 μmol), potassium phosphate (193 mg, 908 μmol), and [1,1′-bis(di-tert-butylphosphino)ferrocene]dichloropalladium(II) (29.6 mg, 45.4 μmol) were dissolved in dioxane (8.00 mL) and water (1.60 mL), and the reaction mixture was stirred and reacted for 12 hours at 80° C. The reaction mixture was filtered through diatomite, and the filtrate was concentrated and purified by preparative silica gel thin layer chromatography (20/1, dichloromethane/methanol, R_{f}=0.27) to obtain compound 84-2. MS-ESI calculated for [M+H]⁺ 546, found 546.

### Step 2

Compound 84-2 (160 mg, 250 μmol) was dissolved in ethyl acetate (2.00 mL), and hydrochloride ethyl acetate solution (4 mol/L, 223 μL) was added thereto at 0° C., and the reaction mixture was stirred and reacted for 12 hours at 11° C. The reaction mixture was filtered. The solid was washed with ethyl acetate (0.4 mL), and the residue was purified by preparative high performance liquid chromatography (chromatographic column: 3_Phenomenex Luna C18 75*30 mm*3 μm; mobile phase: mobile phase A: hydrochloric acid aqueous solution with a volume fraction of 0.05%; mobile phase B: acetonitrile; B %: 2% to 22%, retention time of 6 minutes); the purified solution was adjusted to pH=9 to 10 with ammonia water, extracted with dichloromethane/methanol (10/1, 60 mL), dried over anhydrous sodium sulfate, filtered, and concentrated to obtain compound 84. ¹H NMR (400 MHz, CD₃OD) δ=8.42 (d, J=7.2 Hz, 1H), 7.88 (s, 1H), 7.73-7.70 (m, 1H), 7.63-7.62 (m, 1H), 7.57-7.54 (m, 2H), 7.48-7.45 (m, 1H), 7.34-7.32 (m, 1H), 6.78-6.76 (m, 1H), 4.37-4.21 (m, 2H), 3.14-3.13 (m, 1H), 3.03-2.96 (m, 1H), 2.90-2.84 (m, 1H), 2.08-2.04 (m, 1H), 1.89-1.84 (m, 1H), 1.67-1.57 (m, 1H) 1.50-1.40 (m, 1H). MS-ESI calculated for [M+H]⁺ 446, found 446.

### Example 85

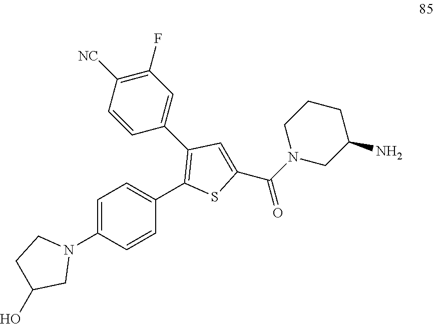 [SMILES: N#Cc1ccc(-c2cc(C(=O)N3CCC[C@@H](N)C3)sc2-c2ccc(N3CCC(O)C3)cc2)cc1F]

Synthetic Route:

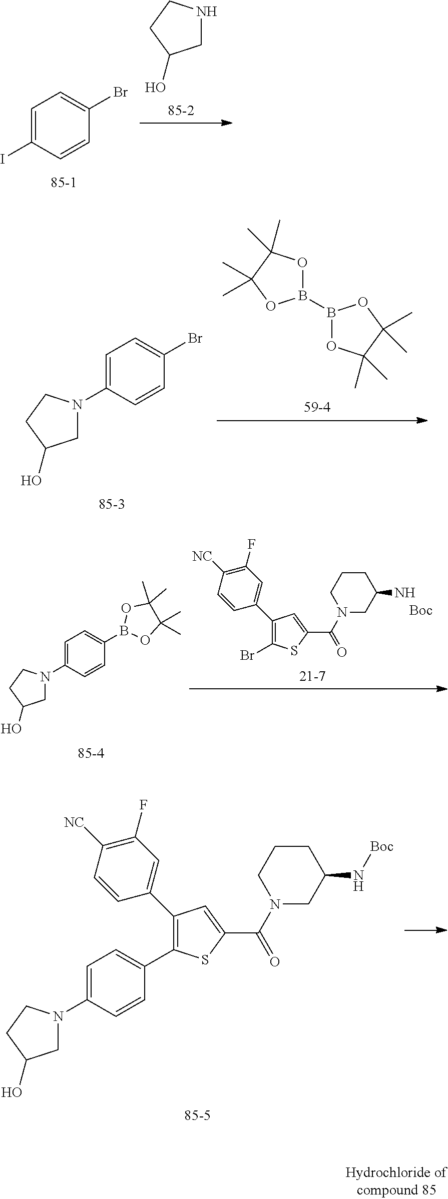

### Step 1

Under nitrogen atmosphere, compound 85-1 (3.0 g, 10.6 mmol) and compound 85-2 (1.85 g, 21.2 mmol) were dissolved in N,N-dimethylethanolamine (30.0 mL), and then potassium phosphate monohydrate (4.88 g, 21.2 mmol) and cuprous iodide (404 mg, 2.12 mmol) were added thereto, and the reaction mixture was stirred and reacted for 60 hours at 55° C. The reaction mixture was added with saturated ammonium chloride (50 mL), and extracted with dichloromethane (50 mL×2). The organic phase was washed with saturated brine (50 mL), dried over anhydrous sodium sulfate, filtered, and concentrated under reduced pressure. The residue was purified by silica gel column chromatography (3/1, petroleum ether/ethyl acetate, R_{f}=0.36) to obtain compound 85-3. ¹H NMR (400 MHz, CD₃OD) δ=7.24 (d, J=8.8 Hz, 2H), 6.47 (d, J=8.8 Hz, 2H), 4.55-4.48 (m, 1H), 3.49-3.36 (m, 2H), 3.30-3.26 (m, 1H), 3.18-3.13 (m, 1H), 2.21-2.09 (m, 1H), 2.07-1.96 (m, 1H). MS-ESI calculated for [M+H]⁺ 242, found 242.

### Step 2

Under nitrogen atmosphere, compound 85-3 (550 mg, 2.27 mmol), bis(pinacolato)diboron (1.73 g, 6.82 mmol), and potassium acetate (669 mg, 6.82 mmol) were dissolved in dioxane (10.0 mL), and the reaction mixture was added with [1,1′-bis(diphenylphosphino)ferrocene]dichloropalladium(II) (166 mg, 0.227 mmol), and the reaction mixture was stirred and reacted for 12 hours at 95° C., and concentrated under reduced pressure. The residue was purified by silica gel column chromatography (1/1, petroleum ether/ethyl acetate, R_{f}=0.60) to obtain compound 85-4. MS-ESI calculated for [M+H]⁺ 290, found 290.

### Step 3

Under nitrogen atmosphere, compound 21-7 (300 mg, 0.590 mmol), compound 85-4 (256 mg, 0.885 mmol), and sodium carbonate (188 mg, 1.77 mmol) were dissolved in dioxane (8.00 mL) and water (2.00 mL), and the reaction mixture was added with [1,1′-bis(di-tert-butylphosphino)ferrocene]dichloropalladium(II) (38.5 mg, 0.059 mmol). The reaction mixture was stirred and reacted for 12 hours at 100° C., added with water (20 mL), and extracted with ethyl acetate (20 mL×1). The organic phase was washed with saturated brine (20 mL×1), dried over anhydrous sodium sulfate, filtered, and concentrated under reduced pressure. The residue was purified by silica gel column chromatography (1/1, petroleum ether/ethyl acetate, R_{f}=0.21) to obtain compound 85-5. MS-ESI calculated for [M+H]⁺ 591, found 591.

### Step 4

Compound 85-5 (220 mg, 0.372 mmol) was dissolved in ethyl acetate (10.0 mL), and hydrochloride ethyl acetate solution (4 mol/L, 10 mL) was added dropwise thereto at 25° C. The reaction mixture was stirred for 1 hour at 25° C., concentrated under reduced pressure, and separated by preparative high performance liquid chromatography (chromatographic column: Phenomenex Genimi NX C18 150*40 mm*5 μm; mobile phase: mobile phase A: hydrochloric acid aqueous solution with a volume fraction of 0.05%; mobile phase B: acetonitrile; B %: 14 to 44%, 10 minutes) to obtain the hydrochloride of compound 85. ¹H NMR (400 MHz, CD₃OD) δ=7.72-7.65 (m, 1H), 7.57 (s, 1H), 7.35-7.26 (m, 4H), 7.07 (d, J=8.4 Hz, 2H), 4.67-4.59 (m, 1H), 4.45-4.35 (m, 1H), 4.21-4.11 (m, 1H), 3.75-3.58 (m, 3H), 3.55-3.36 (m, 4H), 2.35-2.25 (m, 1H), 2.24-2.09 (m, 2H), 1.96-1.86 (m, 1H), 1.84-1.67 (m, 2H). MS-ESI calculated for [M+H]⁺ 491, found 491.

### Example 86

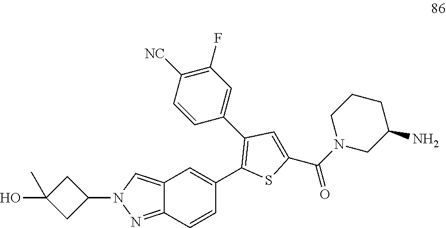 [SMILES: CC1(O)CC(n2cc3cc(-c4sc(C(=O)N5CCC[C@@H](N)C5)cc4-c4ccc(C#N)c(F)c4)ccc3n2)C1]

Synthetic Route:

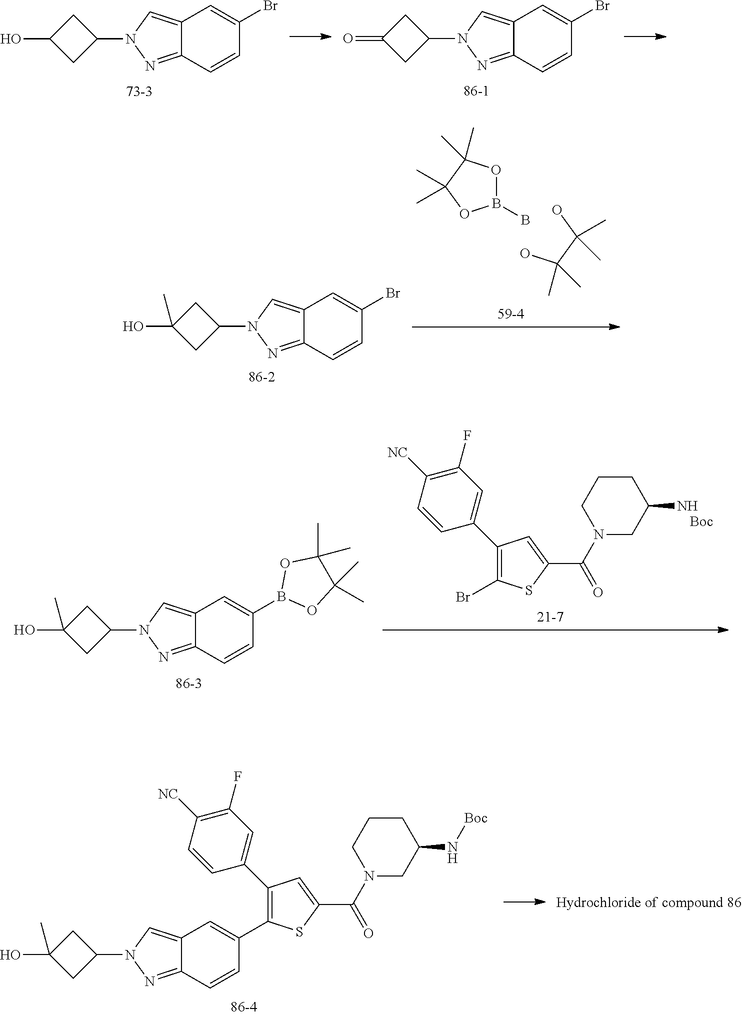

### Step 1

Under nitrogen atmosphere, compound 73-3 (900 mg, 3.37 mmol) was dissolved in anhydrous dichloromethane (10.0 mL), and Dess-Martin periodinane (1.71 g, 4.04 mmol) was added thereto. The reaction mixture was stirred for 12 hours at 25° C., and concentrated under reduced pressure. The residue was purified by silica gel column chromatography (1/1, petroleum ether/ethyl acetate, R_{f}=0.79) to obtain compound 86-1. MS-ESI calculated for [M+H]⁺ 266, found 266.

### Step 2

Under nitrogen atmosphere, compound 86-1 (580 mg, 1.64 mmol) was dissolved in anhydrous tetrahydrofuran (10.0 mL), and methyl lithium (1.6 mol/L ether solution, 2.36 mL) was added thereto at 0° C., and the reaction mixture was stirred for 12 hours at 25° C., added with saturated ammonium chloride aqueous solution (30 mL), and extracted with ethyl acetate (20 mL×1). The organic phase was washed with saturated brine (20 mL×1), dried over anhydrous sodium sulfate, filtered, and concentrated under reduced pressure. The residue was purified by silica gel column chromatography (1/1, petroleum ether/ethyl acetate, R_{f}=0.20) to obtain compound 86-2. ¹H NMR (400 MHz, CDCl₃) δ=7.89 (s, 1H), 7.77 (d, J=1.6 Hz, 1H), 7.57 (d, J=8.8 Hz, 1H), 7.31 (dd, J=2.0, 8.8 Hz, 1H), 4.76-4.68 (m, 1H), 2.82 (d, J=7.6 Hz, 4H), 1.50 (s, 3H). MS-ESI calculated for [M+H]⁺ 281, found 281.

### Step 3

Under nitrogen atmosphere, compound 86-2 (110 mg, 0.391 mmol), bis(pinacolato)diboron (298 mg, 1.17 mmol), and potassium acetate (192 mg, 1.96 mmol) were dissolved in dioxane (10.0 mL), and the reaction mixture was added with [1,1′-bis(diphenylphosphino)ferrocene]dichloropalladium(II) (57.3 mg, 78.3 μmol), and the reaction mixture was stirred and reacted for 12 hours at 95° C., and concentrated under reduced pressure. The residue was purified by silica gel column chromatography (1/1, petroleum ether/ethyl acetate, R_{f}=0.60) to obtain compound 86-3. MS-ESI calculated for [M+H]⁺ 329, found 329.

### Step 4

Under nitrogen atmosphere, compound 21-7 (120 mg, 0.236 mmol), compound 86-3 (85.2 mg, 0.260 mmol), and sodium carbonate (75.1 mg, 0.708 mmol) were dissolved in dioxane (8.00 mL) and water (2.00 mL), and the reaction mixture was added with [1,1′-bis(di-tert-butylphosphino)ferrocene]dichloropalladium(II) (15.4 mg, 0.024 mmol). The reaction mixture was stirred and reacted for 2 hours at 80° C., added with water (20 mL), and extracted with ethyl acetate (20 mL×1). The organic phase was washed with saturated brine (20 mL×1), dried over anhydrous sodium sulfate, filtered, and concentrated under reduced pressure. The residue was purified by silica gel column chromatography (1/1, petroleum ether/ethyl acetate, R_{f}=0.21) to obtain compound 86-4. MS-ESI calculated for [M+H]⁺ 630, found 630.

### Step 5

Under nitrogen atmosphere, compound 86-4 (70.0 mg, 0.111 mmol) was dissolved in anhydrous dichloromethane (5.00 mL), and trimethylsilyl trifluoromethanesulfonate (49.4 mg, 0.222 mmol) was added dropwise at 0° C. The reaction mixture was stirred for 1 hour at 25° C., added with water (1 mL), concentrated under reduced pressure, and then separated by preparative high performance liquid chromatography (chromatographic column: Phenomenex Genimi NX C18 150*40 mm*5 μm; mobile phase: mobile phase A: hydrochloric acid aqueous solution with a volume fraction of 0.05%; mobile phase B: acetonitrile; B %: 10 to 40%, 10 minutes) to obtain the hydrochloride of compound 86. ¹H NMR (400 MHz, CD₃OD) δ=8.51 (s, 1H), 7.82 (s, 1H), 7.67-7.60 (m, 3H), 7.33 (d, J=10.0 Hz, 1H), 7.29-7.20 (m, 2H), 4.87-4.84 (m, 1H), 4.47-4.37 (m, 1H), 4.24-4.14 (m, 1H), 3.56-3.39 (m, 3H), 2.78 (d, J=8.0 Hz, 4H), 2.27-2.16 (m, 1H), 1.98-1.89 (m, 1H), 1.85-1.69 (m, 2H), 1.49 (s, 3H). MS-ESI calculated for [M+H]⁺ 530, found 530.

### Example 87

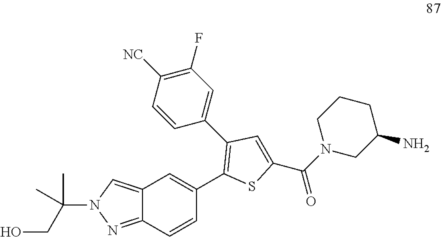 [SMILES: CC(C)(CO)n1cc2cc(-c3sc(C(=O)N4CCC[C@@H](N)C4)cc3-c3ccc(C#N)c(F)c3)ccc2n1]

Synthetic Route:

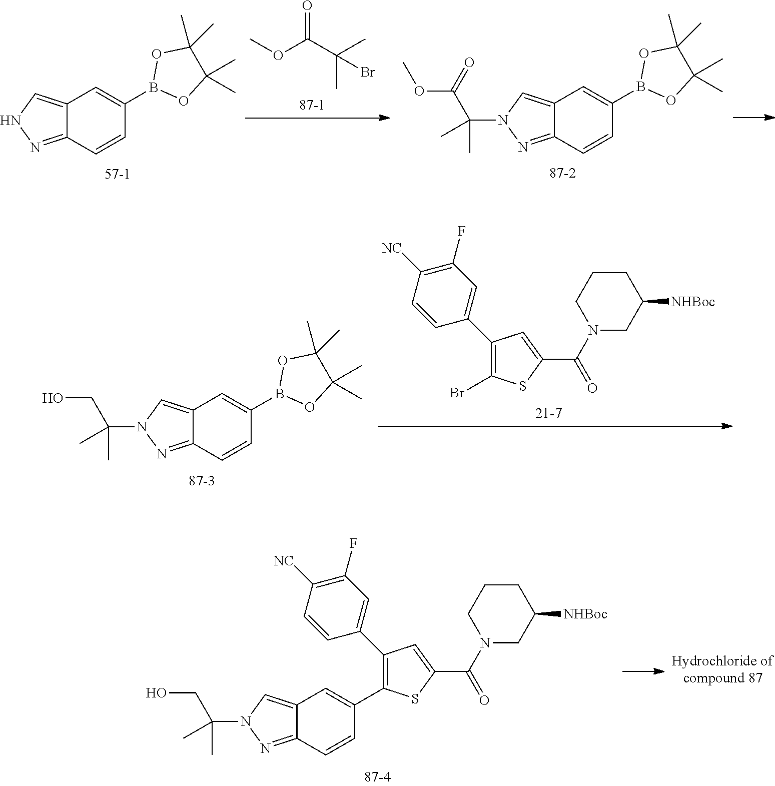

### Step 1

Compounds 57-1 (2.50 g, 10.2 mmol) and 87-1 (5.56 g, 30.7 mmol) were dissolved in acetonitrile (40.0 mL), and potassium carbonate (4.25 g, 30.7 mmol) and potassium iodide (850 mg, 5.12 mmol) were added thereto, and the reaction mixture was stirred and reacted for 12 hours at 85° C. The reaction mixture was concentrated under reduced pressure, and the residue was purified by silica gel column chromatography (1/1, petroleum ether/ethyl acetate, R_{f}=0.38) to obtain compound 87-2. ¹H NMR (400 MHz, CD₃OD) δ=8.48 (s, 1H), 8.24 (s, 1H), 7.60 (d, J=8.8 Hz, 1H), 7.54 (d, J=8.8 Hz, 1H), 3.69 (s, 3H), 1.97 (s, 6H), 1.36 (s, 12H). MS-ESI calculated for [M+H]⁺ 345, found 345.

### Step 2

Lithium borohydride (285 mg, 13.1 mmol) was dissolved in tetrahydrofuran (30.0 mL), and a solution of 87-2 (1.50 g, 4.36 mmol) in tetrahydrofuran (30.0 mL) was added dropwise thereto, and the reaction mixture was stirred and reacted for 0.5 hours at 25° C., then stirred and reacted for another 12 hours at 70° C. The reaction mixture was added dropwise with saturated ammonium chloride aqueous solution (30 mL) at 0° C., and extracted with ethyl acetate (30 mL×2). The organic phases were combined, dried over anhydrous sodium sulfate, filtered, and concentrated to obtain crude compound 87-3, which was used directly in the next reaction step. MS-ESI calculated for [M+H]⁺ 317, found 317.

### Step 3

Under nitrogen atmosphere, compound 21-7 (502 mg, 790 μmol), compound 87-3 (300 mg, 949 μmol), and sodium carbonate (251 mg, 2.37 mmol) were dissolved in dioxane (24.0 mL) and water (6.00 mL), and the reaction mixture was added with [1,1′-bis(diphenylphosphino)ferrocene]dichloropalladium(II) (57.9 mg, 79.1 μmol), and the reaction mixture was stirred and reacted for 12 hours at 100° C. The reaction mixture was concentrated under reduced pressure, and the residue was purified by silica gel column chromatography (1/2, petroleum ether/ethyl acetate, R_{f}=0.37) to obtain compound 87-4. MS-ESI calculated for [M+H]⁺ 618, found 618.

### Step 4

Compound 87-4 (320 mg, 518 μmol) was dissolved in ethyl acetate (10.0 mL), and hydrochloride ethyl acetate solution (4 mol/L, 1.30 mL) was added thereto, and the reaction mixture was stirred and reacted for 2 hours at 25° C. After the reaction mixture was concentrated, the residue was purified by preparative high performance liquid chromatography (chromatographic column: Phenomenex Genimi NX C18 150*40 mm*5 μm, mobile phase A: hydrochloric acid aqueous solution with a volume fraction of 0.05%; mobile phase B: acetonitrile; B %: 5% to 35%, 10 minutes) to obtain the hydrochloride of compound 87. ¹H NMR (400 MHz, CD₃OD) δ=8.67 (s, 1H), 7.89 (s, 1H), 7.68-7.59 (m, 3H), 7.38-7.25 (m, 3H), 4.45-4.39 (m, 1H), 4.20-4.16 (m, 1H), 3.89 (s, 2H), 3.63-3.38 (m, 3H), 2.22-2.18 (m, 1H), 1.94-1.90 (m, 1H), 1.84-1.74 (m, 8H). MS-ESI calculated for [M+H]⁺ 518, found 518.

### Example 88

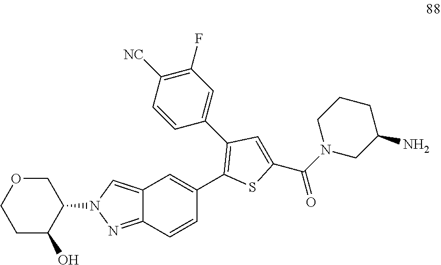 [SMILES: N#Cc1ccc(-c2cc(C(=O)N3CCC[C@@H](N)C3)sc2-c2ccc3nn([C@H]4COCC[C@@H]4O)cc3c2)cc1F]

Synthetic Route:

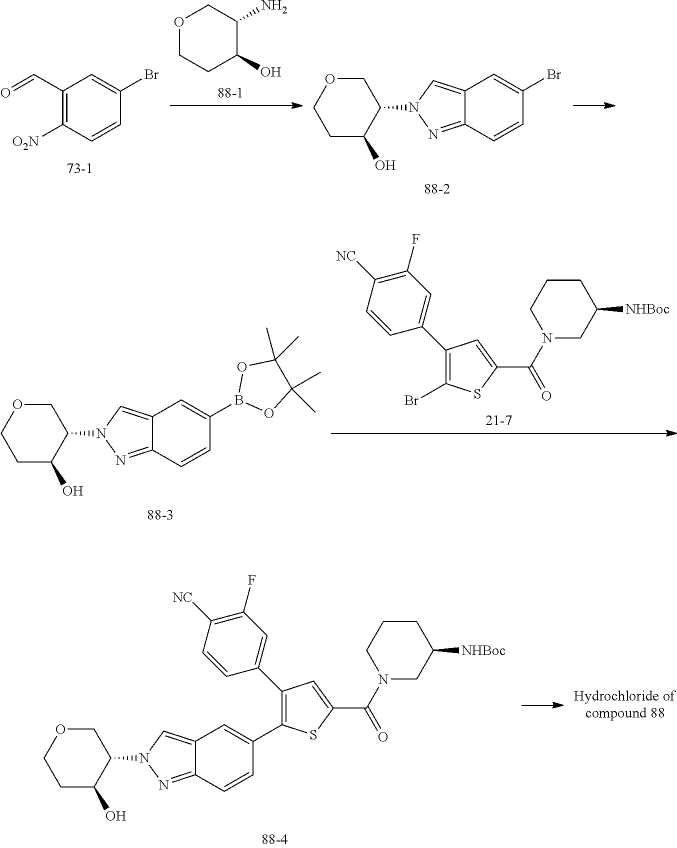

### Step 1

Compound 73-1 (1.50 g, 6.52 mmol) was dissolved in isopropanol (20.0 mL), then 88-1 (917 mg, 7.82 mmol) was added thereto, and the reaction mixture was stirred at 80° C. for 4 hours, then cooled to 25° C., added dropwise with tributylphosphine (4.83 mL, 19.6 mmol), stirred, and reacted for 12 hours at 80° C. The reaction mixture was added with saturated ammonium chloride aqueous solution (50 mL), and extracted with ethyl acetate (50 mL×2). The organic phase was washed with saturated brine (50 mL×1), dried over anhydrous sodium sulfate, filtered, and concentrated under reduced pressure. The residue was purified by silica gel column chromatography (1/1, petroleum ether/ethyl acetate, R_{f}=0.35) to obtain compound 88-2. ¹H NMR (400 MHz, CD₃OD) δ=8.28 (s, 1H), 7.91-7.86 (m, 1H), 7.54 (d, J=9.2 Hz, 1H), 7.35 (dd, J=2.0, 9.2 Hz, 1H), 4.41-4.23 (m, 2H), 4.16-4.09 (m, 1H), 4.07-4.01 (m, 1H), 3.80-3.75 (m, 1H), 3.67-3.55 (m, 1H), 2.12-2.08 (m, 1H), 1.85-1.70 (m, 1H). MS-ESI calculated for [M+H]⁺ 299, found 299.

### Step 2

Under nitrogen atmosphere, compound 88-2 (700 mg, 2.36 mmol), bis(pinacolato)diboron (1.79 g, 7.07 mmol), and potassium acetate (694 mg, 7.07 mmol) were dissolved in dioxane (20.0 mL), and the reaction mixture was added with [1,1′-bis(diphenylphosphino)ferrocene]dichloropalladium(II) (172 mg, 236 μmol). The reaction mixture was stirred and reacted at 100° C. for 12 hours. The reaction mixture was concentrated under reduced pressure, and the residue was purified by silica gel column chromatography (1/1, petroleum ether/ethyl acetate, R_{f}=0.21) to obtain compound 88-3. MS-ESI calculated for [M+H]⁺ 345, found 345.

### Step 3

Under nitrogen atmosphere, compound 21-7 (369 mg, 726 μmol), compound 88-3 (300 mg, 872 μmol), and sodium carbonate (231 mg, 2.18 mmol) were dissolved in dioxane (24.0 mL) and water (6.00 mL), and the reaction mixture was added with [1,1′-bis(diphenylphosphino)ferrocene]dichloropalladium(II) (53.1 mg, 72.6 μmol), and the reaction mixture was stirred and reacted for 12 hours at 100° C. The reaction mixture was concentrated under reduced pressure, and the residue was purified by silica gel column chromatography (1/2, petroleum ether/ethyl acetate, R_{f}=0.24) to obtain compound 88-4. MS-ESI calculated for [M+H]⁺ 646, found 646.

### Step 4

Compound 88-4 (210 mg, 325 μmol) was dissolved in ethyl acetate (10.0 mL), and hydrochloride ethyl acetate solution (4 mol/L, 0.813 mL) was added thereto, and the reaction mixture was stirred and reacted for 2 hours at 25° C. After the reaction mixture was concentrated, the residue was purified by preparative high performance liquid chromatography (chromatographic column: Phenomenex Genimi NX C18 150*40 mm*5 μm, mobile phase A: hydrochloric acid aqueous solution with a volume fraction of 0.05%; mobile phase B: acetonitrile; B %: 15% to 45%, 10 minutes) to obtain the hydrochloride of compound 88. ¹H NMR (400 MHz, CD₃OD) δ=8.52 (s, 1H), 7.85 (s, 1H), 7.71-7.60 (m, 3H), 7.38-7.27 (m, 2H), 7.22 (dd, J=1.6, 8.8 Hz, 1H), 4.50-4.40 (m, 2H), 4.35-4.31 (m, 1H), 4.22-4.15 (m, 2H), 4.12-4.05 (m, 1H), 3.93-3.87 (m, 1H), 3.72-3.60 (m, 1H), 3.58-3.41 (m, 3H), 2.25-2.19 (m, 1H), 2.18-2.09 (m, 1H), 1.90-1.86 (m, 1H), 1.88-1.70 (m, 3H). MS-ESI calculated for [M+H]⁺ 546, found 546.

### Example 89

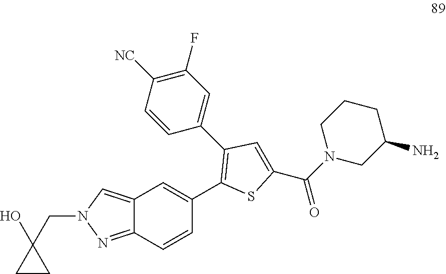 [SMILES: N#Cc1ccc(-c2cc(C(=O)N3CCC[C@@H](N)C3)sc2-c2ccc3nn(CC4(O)CC4)cc3c2)cc1F]

Synthetic Route:

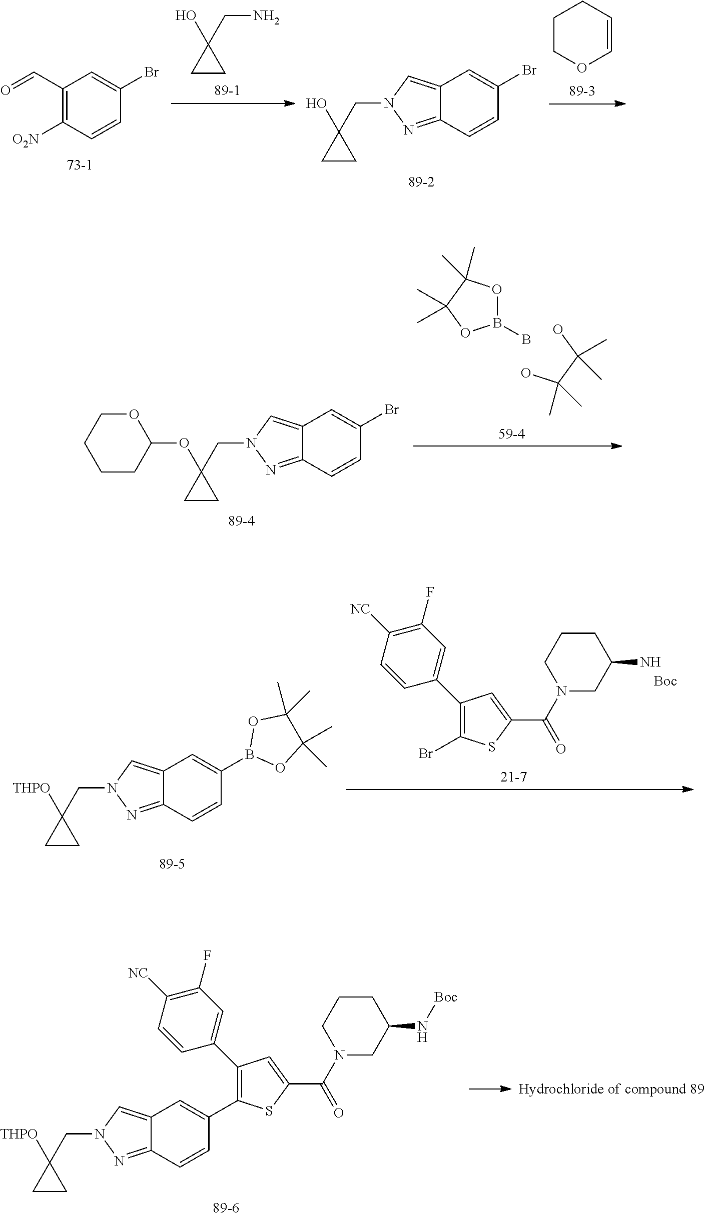

### Step 1

Under nitrogen atmosphere, compound 73-1 (2.0 g, 8.70 mmol) was dissolved in isopropanol (20.0 mL), and 1-(aminomethyl)-1-cyclopropanol (871 mg, 0.478 mmol) was added thereto, and the reaction mixture was stirred for 4 hours at 80° C., added with tributylphosphine (5.28 g, 26.1 mmol) at 0° C., stirred for 12 hours at 80° C., added with saturated ammonium chloride (30 mL), and extracted with ethyl acetate (30 mL×1). The organic phase was washed with saturated brine (30 mL×1), dried over anhydrous sodium sulfate, filtered, concentrated under reduced pressure, and the residue was purified by silica gel column chromatography (1/1, petroleum ether/ethyl acetate, R_{f}=0.20) to obtain compound 89-2. ¹H NMR (400 MHz, CDCl₃) δ=7.94 (s, 1H), 7.84 (s, 1H), 7.61 (d, J=9.2 Hz, 1H), 7.38 (dd, J=1.6, 9.2 Hz, 1H), 4.48 (s, 2H), 1.01-0.95 (m, 2H), 0.81-0.75 (m, 2H). MS-ESI calculated for [M+H]⁺ 267, found 267.

### Step 2

Under nitrogen atmosphere, compound 89-2 (100 mg, 374 μmol) was dissolved in anhydrous dichloromethane (10.0 mL), and then 2,3-dihydropyran (142 mg, 1.68 mmol) and pyridinium p-toluenesulfonate (18.8 mg, 74.9 μmol) were added thereto. The reaction mixture was stirred for 12 hours at 25° C., and concentrated under reduced pressure. The residue was purified by silica gel column chromatography (1/1, petroleum ether/ethyl acetate, R_{f}=0.68) to obtain compound 89-4. MS-ESI calculated for [M+H]⁺ 353, found 353.

### Step 3

Under nitrogen atmosphere, compound 89-4 (120 mg, 0.342 mmol), bis(pinacolato)diboron (260 mg, 1.02 mmol), and potassium acetate (168 mg, 1.71 mmol) were dissolved in dioxane (10.0 mL), and the reaction mixture was added with [1,1′-bis(diphenylphosphino)ferrocene]dichloropalladium(II) (50.0 mg, 68.3 μmol), and the reaction mixture was stirred and reacted for 12 hours at 95° C., and concentrated under reduced pressure. The residue was purified by silica gel column chromatography (1/1, petroleum ether/ethyl acetate, R_{f}=0.60) to obtain compound 89-5. ¹H NMR (400 MHz, CDCl₃) δ=8.30-8.26 (m, 2H), 7.68-7.64 (m, 2H), 5.04 (d, J=14.4 Hz, 1H), 4.74-4.69 (m, 1H), 4.31 (d, J=14.4 Hz, 1H), 3.85-3.75 (m, 1H), 3.51-3.42 (m, 1H), 1.78-1.68 (m, 1H), 1.64-1.55 (m, 1H), 1.52-1.42 (m, 4H), 1.37 (s, 12H), 1.21-1.15 (m, 1H), 1.02-0.91 (m, 2H), 0.85-0.79 (m, 1H). MS-ESI calculated for [M+H]⁺ 399, found 399.

### Step 4

Under nitrogen atmosphere, compound 21-7 (150 mg, 0.295 mmol), compound 89-5 (123 mg, 0.310 mmol), and sodium carbonate (93.8 mg, 0.885 mmol) were dissolved in dioxane (8.00 mL) and water (2.00 mL), and the reaction mixture was added with [1,1′-bis(diphenylphosphino)ferrocene]dichloropalladium(II) (21.6 mg, 25.9 μmol). The reaction mixture was stirred and reacted for 12 hours at 100° C., added with water (20 mL), and extracted with ethyl acetate (20 mL×1). The organic phase was washed with saturated brine (20 mL×1), dried over anhydrous sodium sulfate, filtered, and concentrated under reduced pressure. The residue was purified by silica gel column chromatography (1/1, petroleum ether/ethyl acetate, R_{f}=0.30) to obtain compound 89-6. ¹H NMR (400 MHz, CDCl₃) δ=8.32 (s, 1H), 7.75 (s, 1H), 7.70-7.52 (m, 2H), 7.46-7.36 (m, 1H), 7.34-7.27 (m, 1H), 7.20-6.95 (m, 2H), 5.15-5.03 (m, 1H), 4.79-4.57 (m, 3H), 4.54-4.37 (m, 1H), 4.23 (d, J=14.4 Hz, 1H), 3.93-3.83 (m, 1H), 3.75-3.59 (m, 1H), 3.56-3.45 (m, 1H), 3.07-2.82 (m, 2H), 2.12-2.06 (m, 1H), 1.95-1.82 (m, 2H), 1.83-1.75 (m, 1H), 1.69-1.59 (m, 2H), 1.54-1.49 (m, 3H), 1.47 (s, 9H), 1.21-1.11 (m, 1H), 1.04-0.91 (m, 2H), 0.88-0.80 (m, 1H). MS-ESI calculated for [M+H]⁺ 700, found 700.

### Step 5

Compound 89-6 (150 mg, 0.214 mmol) was dissolved in ethyl acetate (5.00 mL), and hydrochloride ethyl acetate solution (4 mol/L, 5 mL) was added dropwise thereto at 25° C. The reaction mixture was stirred for 1 hour at 25° C., concentrated under reduced pressure, and purified by preparative high performance liquid chromatography (chromatographic column: Phenomenex Genimi NX C18 150*40 mm*5 μm; mobile phase: mobile phase A: hydrochloric acid aqueous solution with a volume fraction of 0.05%; mobile phase B: acetonitrile; B %: 10 to 40%, 10 minutes) to obtain the hydrochloride of compound 89. ¹H NMR (400 MHz, CD₃OD) δ=8.88 (s, 1H), 8.04 (s, 1H), 7.73 (d, J=8.8 Hz, 1H), 7.69-7.64 (m, 2H), 7.49 (d, J=8.8 Hz, 1H), 7.36 (d, J=10.4 Hz, 1H), 7.30 (d, J=8.0 Hz, 1H), 4.72 (s, 2H), 4.51-4.39 (m, 1H), 4.25-4.11 (m, 1H), 3.60-3.41 (m, 3H), 2.27-2.18 (m, 1H), 2.00-1.89 (m, 1H), 1.87-1.69 (m, 2H), 1.02-0.96 (m, 2H), 0.96-0.89 (m, 2H). MS-ESI calculated for [M+H]⁺ 516, found 516.

### Example 90

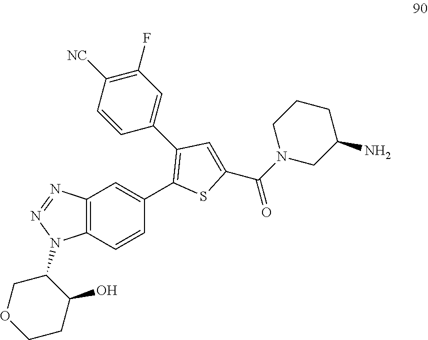 [SMILES: N#Cc1ccc(-c2cc(C(=O)N3CCC[C@@H](N)C3)sc2-c2ccc3c(c2)nnn3[C@H]2COCC[C@@H]2O)cc1F]

Synthetic Route:

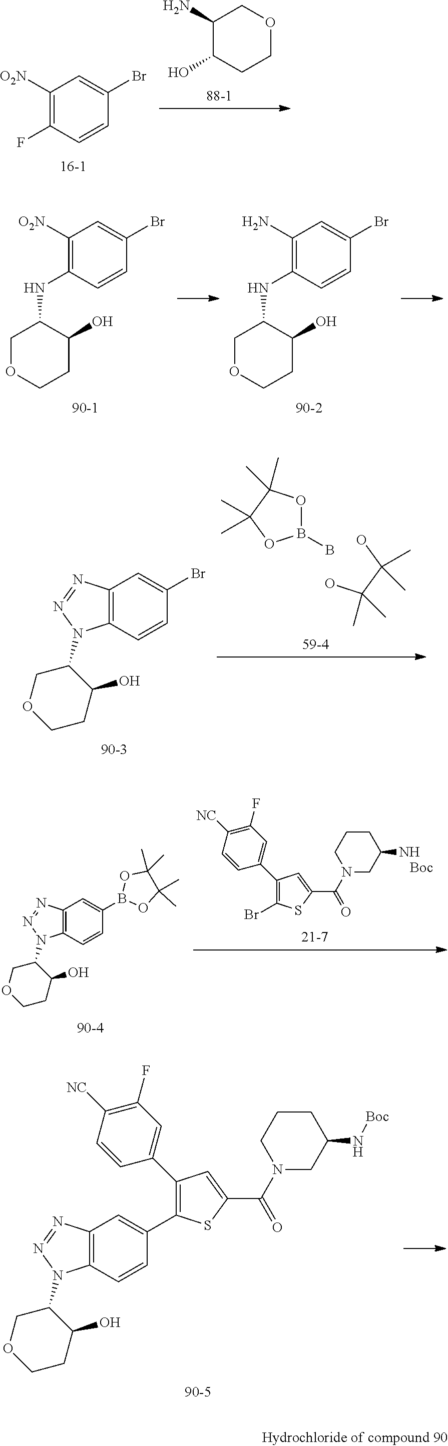

### Step 1

Under nitrogen atmosphere, compound 16-1 (2.00 g, 9.09 mmol) and (3S,4S)-3-aminotetrahydro-2H-pyran-4-ol (1.28 g, 10.9 mmol) were dissolved in N,N-dimethylformamide (10.0 mL), and N,N-diisopropylethylamine (2.35 g, 18.2 mmol) was added thereto, and the reaction mixture was stirred and reacted for 12 hours at 80° C., added with water (20 mL), extracted with ethyl acetate (20 mL×1). The organic phase was washed with saturated brine (20 mL×1), dried over anhydrous sodium sulfate, filtered, and concentrated under reduced pressure. The residue was purified by silica gel column chromatography (1/1, petroleum ether/ethyl acetate, R_{f}=0.19) to obtain compound 90-1. MS-ESI calculated for [M+H]⁺ 319, found 319.

### Step 2

Compound 90-1 (1.30 g, 4.10 mmol) was dissolved in concentrated hydrochloric acid (15.0 mL), and tin dichloride (5.55 g, 24.6 mmol) was added thereto, and the reaction mixture was stirred and reacted for 12 hours at 25° C. The reaction mixture was adjusted to pH=13 with sodium hydroxide aqueous solution (10 mol/L), and extracted with ethyl acetate (50 mL×1). The organic phase was washed with saturated brine (50 mL×1), dried over anhydrous sodium sulfate, filtered, and concentrated under reduced pressure to obtain compound 90-2. MS-ESI calculated for [M+H]⁺ 287, found 287.

### Step 3

Compound 90-2 (1.10 g, 3.83 mmol) was dissolved in acetic acid (16.0 mL), and a solution of sodium nitrite (396 mg, 5.75 mmol) in water (8.00 mL) was added dropwise thereto, and the reaction mixture was stirred for 1 hour at 25° C., added with water (30 mL), and extracted with ethyl acetate (50 mL×2). The organic phase was sequentially washed with saturated sodium bicarbonate aqueous solution (100 mL×3) and saturated brine (100 mL×1), dried over anhydrous sodium sulfate, filtered, and concentrated under reduced pressure. The residue was purified by silica gel column chromatography (1/1, petroleum ether/ethyl acetate, R_{f}=0.51) to obtain compound 90-3. MS-ESI calculated for [M+H]⁺ 298, found 298.

### Step 4

Under nitrogen atmosphere, compound 90-3 (380 mg, 1.27 mmol), bis(pinacolato)diboron (1.02 g, 4.02 mmol), and potassium acetate (658 mg, 6.70 mmol) were dissolved in dioxane (10.0 mL), and the reaction mixture was added with [1,1′-bis(diphenylphosphino)ferrocene]dichloropalladium(II) (98.1 mg, 0.134 mmol), and the reaction mixture was stirred and reacted for 12 hours at 100° C., and concentrated under reduced pressure. The residue was purified by silica gel column chromatography (1/1, petroleum ether/ethyl acetate, R_{f}=0.21) to obtain compound 90-4. MS-ESI calculated for [M+H]⁺ 346, found 346.

### Step 5

Under nitrogen atmosphere, compound 21-7 (400 mg, 0.787 mmol), compound 90-4 (407 mg, 1.18 mmol), and sodium carbonate (250 mg, 2.36 mmol) were dissolved in dioxane (8.00 mL) and water (2.00 mL), and the reaction mixture was added with [1,1′-bis(di-tert-butylphosphino)ferrocene]dichloropalladium(II) (57.6 mg, 78.7 μmol). The reaction mixture was stirred and reacted for 2 hours at 80° C., added with water (20 mL), and extracted with ethyl acetate (20 mL×1). The organic phase was washed with saturated brine (20 mL×1), dried over anhydrous sodium sulfate, filtered, and concentrated under reduced pressure. The residue was purified by silica gel column chromatography (1/1, petroleum ether/ethyl acetate, R_{f}=0.21) to obtain compound 90-5. MS-ESI calculated for [M+H]⁺ 647, found 647.

### Step 6

Compound 90-5 (400 mg, 0.214 mmol) was dissolved in ethyl acetate (10.0 mL), and hydrochloride ethyl acetate solution (4 mol/L, 10.0 mL) was added dropwise thereto at 25° C. The reaction mixture was stirred for 1 hour at 25° C., concentrated under reduced pressure, and separated by preparative high performance liquid chromatography (chromatographic column: Phenomenex Genimi NX C18 150*40 mm*5 μm; mobile phase: mobile phase A: hydrochloric acid aqueous solution with a volume fraction of 0.05%; mobile phase B: acetonitrile; B %: 5 to 35%, 10 minutes) to obtain the hydrochloride of compound 90. ¹H NMR (400 MHz, CD₃OD) δ=7.96 (s, 1H), 7.86 (d, J=8.8 Hz, 1H), 7.67-7.60 (m, 2H), 7.42 (d, J=9.2 Hz, 1H), 7.34 (d, J=10.0 Hz, 1H), 7.25 (d, J=8.8 Hz, 1H), 4.78-4.68 (m, 1H), 4.49-4.41 (m, 1H), 4.40-4.32 (m, 1H), 4.24-4.13 (m, 2H), 4.12-3.99 (m, 2H), 3.67 (m, 1H), 3.60-3.41 (m, 3H), 2.28-2.12 (m, 2H), 1.99-1.67 (m, 4H). MS-ESI calculated for [M+H]⁺ 547, found 547.

### Example 91

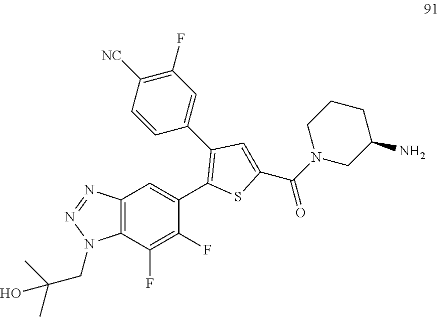 [SMILES: CC(C)(O)Cn1nnc2cc(-c3sc(C(=O)N4CCC[C@@H](N)C4)cc3-c3ccc(C#N)c(F)c3)c(F)c(F)c21]

Synthetic Route:

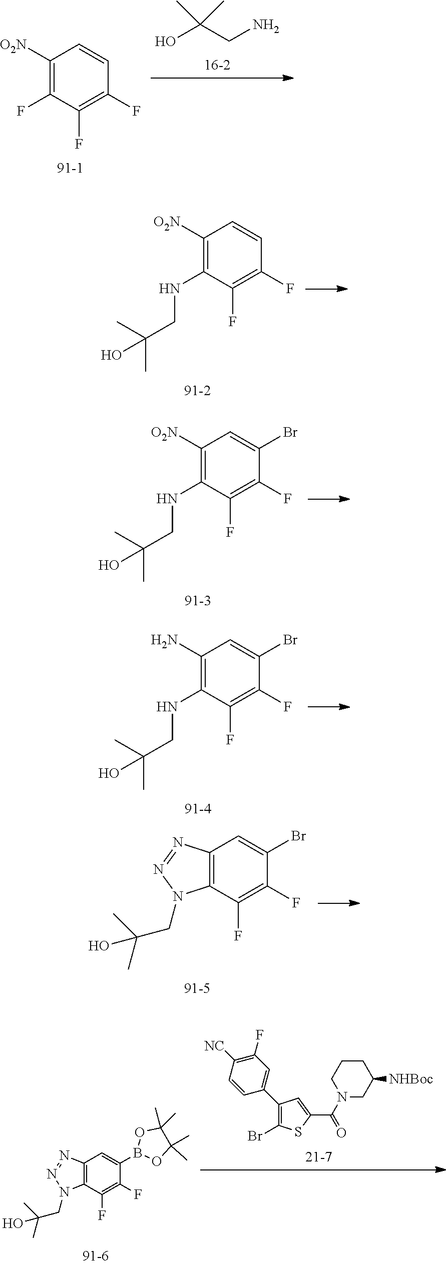

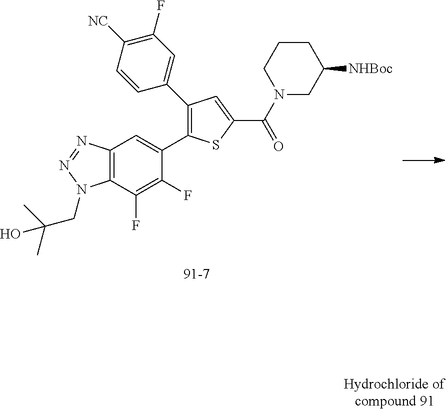 [SMILES: CC(C)(O)Cn1nnc2cc(-c3sc(C(=O)N4CCC[C@@H](NC(=O)OC(C)(C)C)C4)cc3-c3ccc(C#N)c(F)c3)c(F)c(F)c21]

### Step 1

Compounds 91-1 (3.05 g, 17.2 mmol) and 16-2 (1.84 g, 20.7 mmol) were dissolved in N,N-dimethylformamide (20.0 mL), and N,N-diisopropylethylamine (6.68 g, 51.7 mmol) was added thereto, and the reaction mixture was stirred and reacted for 12 hours at 80° C. The reaction mixture was added with water (50 mL), and extracted with ethyl acetate (50 mL×2). The organic phase was washed with saturated brine (50 mL×1), dried over anhydrous sodium sulfate, filtered, and concentrated under reduced pressure. The residue was purified by silica gel column chromatography (4/1, petroleum ether/ethyl acetate, R_{f}=0.41) to obtain compound 91-2. ¹H NMR (400 MHz, CD₃OD) δ=8.13-8.01 (m, 1H), 6.68-6.54 (m, 1H), 3.58 (d, J=3.2 Hz, 2H), 1.28 (s, 6H). MS-ESI calculated for [M+H]⁺ 247, found 247.

### Step 2

Compound 91-2 (1.50 g, 6.09 mmol) was dissolved in N,N-dimethylformamide (20.0 mL). N-Bromosuccinimide (1.41 g, 7.92 mmol) was added thereto, and the reaction mixture was stirred and reacted for 2 hours at 90° C. The reaction mixture was added with water (50 mL), and extracted with ethyl acetate (50 mL×2). The organic phase was washed with saturated brine (50 mL×1), dried over anhydrous sodium sulfate, filtered, and concentrated under reduced pressure. The residue was purified by silica gel column chromatography (3/1, petroleum ether/ethyl acetate, R_{f}=0.60) to obtain compound 91-3. ¹H NMR (400 MHz, CD₃OD) δ=8.27-8.22 (m, 1H), 3.59 (d, J=3.6 Hz, 2H), 1.32-1.26 (m, 6H). MS-ESI calculated for [M+H]⁺ 325, found 325.

### Step 3

Compound 91-3 (1.70 g, 5.23 mmol) was dissolved in concentrated hydrochloric acid (20.0 mL), then tin dichloride (7.08 g, 31.4 mmol) was added thereto, and the reaction mixture was stirred and reacted for 4 hours at 25° C. The reaction mixture was adjusted to pH=13 with sodium hydroxide aqueous solution (4 mol/L), and extracted with ethyl acetate (50 mL×2). The organic phase was washed with saturated brine (50 mL×1), dried over anhydrous sodium sulfate, filtered, and concentrated under reduced pressure to obtain crude compound 91-4, which was used directly in the next reaction step. MS-ESI calculated for [M+H]⁺ 295, found 295.

### Step 4

Compound 91-4 (1.50 g, 5.08 mmol) was dissolved in acetic acid (20.0 mL), and a solution of sodium nitrite (526 mg, 7.62 mmol) in water (10.0 mL) was added dropwise thereto, and the reaction mixture was stirred for 2 hours at 25° C. The reaction mixture was added with water (50 mL), and extracted with ethyl acetate (50 mL×2). The organic phase was washed with saturated ammonium chloride aqueous solution (50 mL), dried over anhydrous sodium sulfate, filtered, and concentrated under reduced pressure. The residue was purified by silica gel column chromatography (2/1, petroleum ether/ethyl acetate, R_{f}=0.59) to obtain compound 91-5. ¹H NMR (400 MHz, CD₃OD) δ=8.20-8.14 (m, 1H), 4.75 (s, 2H), 1.34-1.26 (m, 6H). MS-ESI calculated for [M+H]⁺ 306, found 306.

### Step 5

Under nitrogen atmosphere, compound 91-5 (600 mg, 1.96 mmol), bis(pinacolato)diboron (1.49 g, 5.88 mmol), and potassium acetate (577 mg, 5.88 mmol) were dissolved in dioxane (20.0 mL), and the reaction mixture was added with [1,1′-bis(diphenylphosphino)ferrocene]dichloropalladium(II) (143 mg, 196 μmol). The reaction mixture was stirred and reacted at 100° C. for 12 hours. The reaction mixture was concentrated under reduced pressure, and the residue was purified by silica gel column chromatography (2/1, petroleum ether/ethyl acetate, R_{f}=0.42) to obtain compound 91-6. ¹H NMR (400 MHz, CD₃OD) δ=8.13-8.10 (m, 1H), 4.72 (s, 2H), 1.40 (s, 12H), 1.28 (s, 6H). MS-ESI calculated for [M+H]⁺ 354, found 354.

### Step 6

Under nitrogen atmosphere, compound 21-7 (300 mg, 590 μmol), compound 91-6 (250 mg, 707 μmol), and sodium carbonate (188 mg, 1.77 mmol) were dissolved in dioxane (16.0 mL) and water (4.00 mL), and the reaction mixture was added with [1,1′-bis(diphenylphosphino)ferrocene]dichloropalladium(II) (46.3 mg, 43.2 μmol), and the reaction mixture was stirred and reacted for 12 hours at 100° C. The reaction mixture was concentrated under reduced pressure, and the residue was purified by silica gel column chromatography (1/1, petroleum ether/ethyl acetate, R_{f}=0.24) to obtain compound 91-7. MS-ESI calculated for [M+H]⁺ 655, found 655.

### Step 7

Compound 91-7 (240 mg, 367 μmol) was dissolved in ethyl acetate (10.0 mL), and hydrochloride ethyl acetate solution (4 mol/L, 0.916 mL) was added thereto, and the reaction mixture was stirred and reacted for 2 hours at 25° C. After the reaction mixture was concentrated, the residue was purified by preparative high performance liquid chromatography (chromatographic column: Phenomenex Genimi NX C18 150*40 mm*5 μm, mobile phase A: hydrochloric acid aqueous solution with a volume fraction of 0.05%; mobile phase B: acetonitrile; B %: 10% to 40%, 10 minutes) to obtain the hydrochloride of compound 91. ¹H NMR (400 MHz, CD₃OD) δ=8.10-7.98 (m, 1H), 7.72 (s, 1H), 7.70-7.64 (m, 1H), 7.43-7.36 (m, 1H), 7.29-7.23 (m, 1H), 4.76 (s, 2H), 4.43-3.99 (m, 1H), 4.19-4.17 (m, 1H), 3.65-3.41 (m, 3H), 2.24-2.20 (m, 1H), 2.00-1.89 (m, 1H), 1.86-1.71 (m, 2H), 1.31 (s, 6H). MS-ESI calculated for [M+H]⁺ 555, found 555.

### Example 92

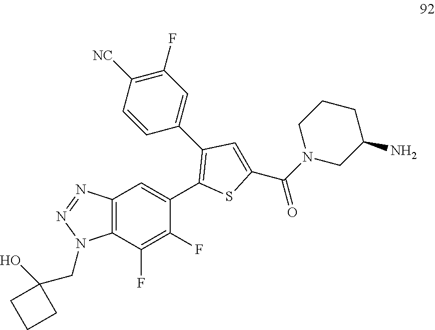 [SMILES: N#Cc1ccc(-c2cc(C(=O)N3CCC[C@@H](N)C3)sc2-c2cc3nnn(CC4(O)CCC4)c3c(F)c2F)cc1F]

Synthetic Route:

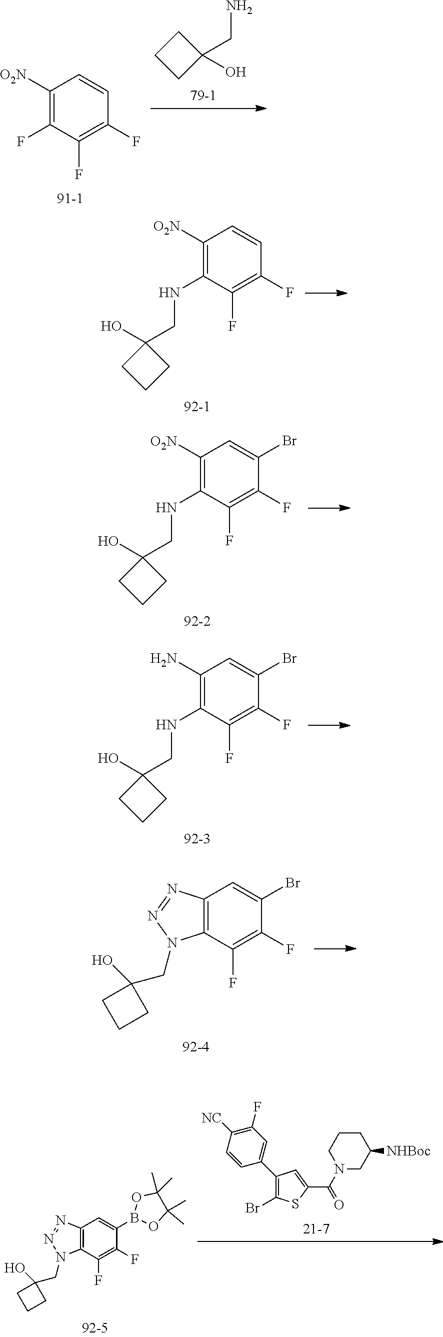

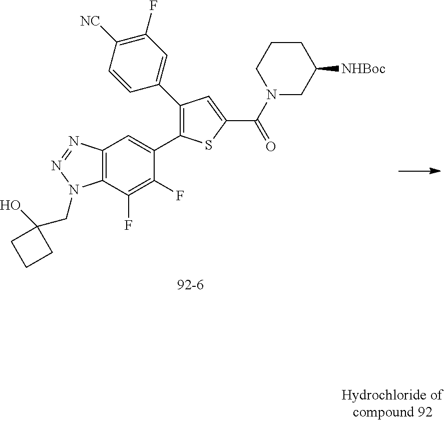 [SMILES: CC(C)(C)OC(=O)N[C@@H]1CCCN(C(=O)c2cc(-c3ccc(C#N)c(F)c3)c(-c3cc4nnn(CC5(O)CCC5)c4c(F)c3F)s2)C1]

### Step 1

Compounds 91-1 (3.00 g, 16.9 mmol) and 79-1 (2.06 g, 20.3 mmol) were dissolved in N,N-dimethylformamide (20.0 mL), and N,N-diisopropylethylamine (6.57 g, 50.8 mmol) was added thereto, and the reaction mixture was stirred and reacted for 12 hours at 80° C. The reaction mixture was added with water (50 mL), and extracted with ethyl acetate (50 mL×2). The organic phase was washed with saturated brine (50 mL×1), dried over anhydrous sodium sulfate, filtered, and concentrated under reduced pressure. The residue was purified by silica gel column chromatography (2/1, petroleum ether/ethyl acetate, R_{f}=0.38) to obtain compound 92-1. ¹H NMR (400 MHz, CD₃OD) δ=8.06-7.94 (m, 1H), 6.68-6.52 (m, 1H), 3.73 (d, J=3.2 Hz, 2H), 2.21-2.05 (m, 4H), 1.91-1.72 (m, 1H), 1.70-1.53 (m, 1H). MS-ESI calculated for [M+H]⁺ 259, found 259.

### Step 2

Compound 92-1 (2.00 g, 7.75 mmol) was dissolved in N,N-dimethylformamide (20.0 mL). N-Bromosuccinimide (1.79 g, 10.1 mmol) was added thereto, and the reaction mixture was stirred and reacted for 2 hours at 90° C. The reaction mixture was added with water (100 mL), and extracted with ethyl acetate (100 mL×2). The organic phase was washed with saturated brine (100 mL×1), dried over anhydrous sodium sulfate, filtered, and concentrated under reduced pressure. The residue was purified by silica gel column chromatography (3/1, petroleum ether/ethyl acetate, R_{f}=0.64) to obtain compound 92-2. ¹H NMR (400 MHz, CD₃OD) δ=8.24-8.20 (m, 1H), 3.72 (d, J=3.6 Hz, 2H), 2.16-2.06 (m, 4H), 1.86-1.74 (m, 1H), 1.68-1.55 (m, 1H). MS-ESI calculated for [M+H]⁺ 337 and 339, found 337 and 339.

### Step 3

Compound 92-2 (2.04 g, 6.05 mmol) was dissolved in concentrated hydrochloric acid (30.0 mL), then tin dichloride (8.19 g, 36.3 mmol) was added thereto, and the reaction mixture was stirred and reacted for 2 hours at 25° C. The reaction mixture was adjusted to pH=13 with sodium hydroxide aqueous solution (4 mol/L), and extracted with ethyl acetate (50 mL×2). The organic phase was washed with saturated brine (50 mL×1), dried over anhydrous sodium sulfate, filtered, and concentrated under reduced pressure to obtain crude compound 92-3, which was used directly in the next reaction step. MS-ESI calculated for [M+H]⁺ 307 and 309, found 307 and 309.

### Step 4

Compound 92-3 (1.66 g, 5.40 mmol) was dissolved in acetic acid (20.0 mL), and a solution of sodium nitrite (559 mg, 8.11 mmol) in water (10 mL) was added dropwise thereto, and the reaction mixture was stirred for 1 hour at 25° C. The reaction mixture was added with water (50 mL), and extracted with ethyl acetate (50 mL×2). The organic phase was washed with saturated ammonium chloride aqueous solution (50 mL), dried over anhydrous sodium sulfate, filtered, and concentrated under reduced pressure. The residue was purified by silica gel column chromatography (2/1, petroleum ether/ethyl acetate, R_{f}=0.59) to obtain compound 92-4. ¹H NMR (400 MHz, CD₃OD) δ=8.15-8.11 (m, 1H), 4.87 (s, 2H), 2.3-2.30 (m, 2H), 2.12-1.99 (m, 2H), 1.93-1.67 (m, 2H). MS-ESI calculated for [M+H]⁺ 318 and 320, found 318 and 320.

### Step 5

Under nitrogen atmosphere, compound 92-4 (0.500 g, 1.57 mmol), bis(pinacolato)diboron (1.20 g, 4.72 mmol), and potassium acetate (463 mg, 4.72 mmol) were dissolved in dioxane (20.0 mL), and the reaction mixture was added with [1,1′-bis(diphenylphosphino)ferrocene]dichloropalladium(II) (115 mg, 157 μmol). The reaction mixture was stirred and reacted at 100° C. for 12 hours. The reaction mixture was concentrated under reduced pressure, and the residue was purified by silica gel column chromatography (2/1, petroleum ether/ethyl acetate, R_{f}=0.33) to obtain compound 92-5. MS-ESI calculated for [M+H]⁺ 366, found 366.

### Step 6

Under nitrogen atmosphere, compound 21-7 (419 mg, 826 μmol), compound 92-5 (603 mg, 1.65 mmol), and sodium carbonate (262 mg, 2.48 mmol) were dissolved in dioxane (20.0 mL) and water (5.00 mL), and the reaction mixture was added with [1,1′-bis(diphenylphosphino)ferrocene]dichloropalladium(II) (60.4 mg, 82.6 μmol), and the reaction mixture was stirred and reacted for 12 hours at 100° C. The reaction mixture was concentrated under reduced pressure, and the residue was purified by silica gel column chromatography (1/1, petroleum ether/ethyl acetate, R_{f}=0.31) to obtain compound 92-6. MS-ESI calculated for [M+H]⁺ 667, found 667.

### Step 7

Compound 92-6 (200 mg, 300 μmol) was dissolved in ethyl acetate (10.0 mL), and hydrochloride ethyl acetate solution (4 mol/L, 0.750 mL) was added thereto, and the reaction mixture was stirred and reacted for 2 hours at 25° C. After the reaction mixture was concentrated, the residue was purified by preparative high performance liquid chromatography (chromatographic column: Phenomenex Genimi NX C18 150*40 mm*5 μm, mobile phase A: hydrochloric acid aqueous solution with a volume fraction of 0.05%; mobile phase B: acetonitrile; B %: 10% to 40%, 10 minutes) to obtain the hydrochloride of compound 92. ¹H NMR (400 MHz, CD₃OD) δ=7.98-7.94 (m, 1H), 7.70 (s, 1H), 7.67-7.62 (m, 1H), 7.38 (dd, J=1.2, 10.4 Hz, 1H), 7.23 (dd, J=1.6, 8.0 Hz, 1H), 4.86 (s, 2H), 4.44-4.36 (m, 1H), 4.20-4.12 (m, 1H), 3.69-3.39 (m, 3H), 2.41-2.29 (m, 2H), 2.24-2.20 (m, 1H), 2.13-2.00 (m, 2H), 1.98-1.67 (m, 5H). MS-ESI calculated for [M+H]⁺ 567, found 567.

### Example 93

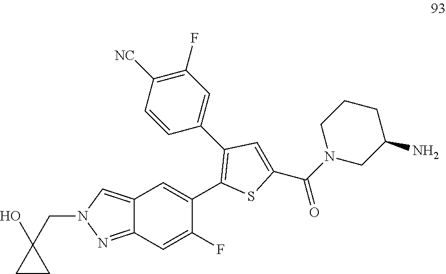 [SMILES: N#Cc1ccc(-c2cc(C(=O)N3CCC[C@@H](N)C3)sc2-c2cc3cn(CC4(O)CC4)nc3cc2F)cc1F]

Synthetic Route:

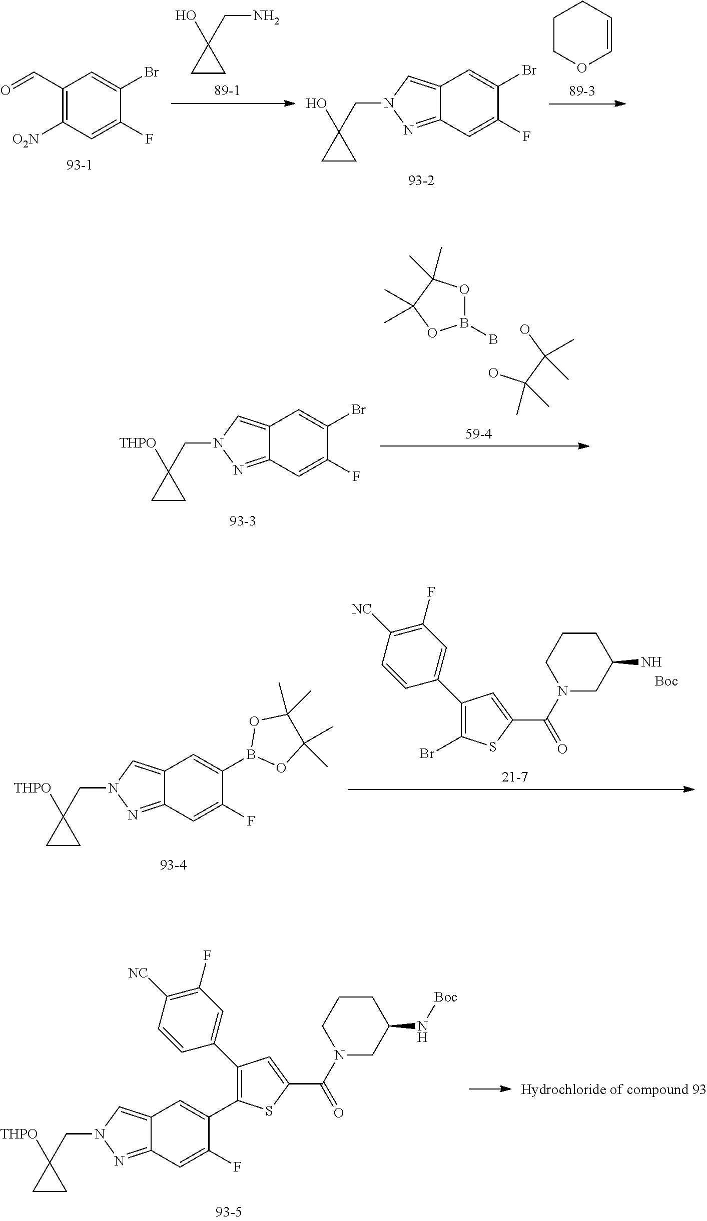

### Step 1

Under nitrogen atmosphere, compound 93-1 (1.50 g, 6.05 mmol) was dissolved in isopropanol (15.0 mL), and 1-(aminomethyl)-1-cyclopropanol (606 mg, 6.96 mmol) was added thereto, and the reaction mixture was stirred for 4 hours at 80° C., added with tributylphosphine (5.28 g, 26.1 mmol) at 0° C., stirred for 12 hours at 80° C., added with saturated ammonium chloride (30 mL), and extracted with ethyl acetate (30 mL×1). The organic phase was washed with saturated brine (30 mL×1), dried over anhydrous sodium sulfate, filtered, concentrated under reduced pressure, and the residue was purified by silica gel column chromatography (1/1, petroleum ether/ethyl acetate, R_{f}=0.20) to obtain compound 93-2. ¹H NMR (400 MHz, CDCl₃) δ=7.95 (s, 1H), 7.91 (d, J=6.4 Hz, 1H), 7.41 (d, J=9.6 Hz, 1H), 4.46 (s, 2H), 1.00-0.95 (m, 2H), 0.80-0.75 (m, 2H). MS-ESI calculated for [M+H]⁺ 287, found 287.

### Step 2

Under nitrogen atmosphere, compound 93-2 (1.40 g, 4.91 mmol) was dissolved in anhydrous dichloromethane (5.00 mL), and then 2,3-dihydropyran (1.86 g, 22.1 mmol) and pyridinium p-toluenesulfonate (247 mg, 0.982 mmol) were added thereto. The reaction mixture was stirred for 12 hours at 25° C., and concentrated under reduced pressure. The residue was separated by silica gel column chromatography (1/1, petroleum ether/ethyl acetate, R_{f}=0.68) to obtain compound 93-3. MS-ESI calculated for [M+H]⁺ 369, found 369.

### Step 3

Under nitrogen atmosphere, compound 93-3 (400 mg, 1.08 mmol), bis(pinacolato)diboron (825 mg, 3.25 mmol), and potassium acetate (319 mg, 3.25 mmol) were dissolved in dioxane (10.0 mL), and the reaction mixture was added with [1,1′-bis(diphenylphosphino)ferrocene]dichloropalladium(II) (79.3 mg, 0.108 mmol), and the reaction mixture was stirred and reacted for 12 hours at 95° C., and concentrated under reduced pressure. The residue was purified by silica gel column chromatography (1/1, petroleum ether/ethyl acetate, R_{f}=0.60) to obtain compound 93-4. MS-ESI calculated for [M+H]⁺ 417, found 417.

### Step 4

Under nitrogen atmosphere, compound 21-7 (310 mg, 0.610 mmol), compound 93-4 (381 mg, 0.915 mmol), and sodium carbonate (194 mg, 1.83 mmol) were dissolved in dioxane (8.00 mL) and water (2.00 mL), and the reaction mixture was added with [1,1′-bis(diphenylphosphino)ferrocene]dichloropalladium(II) (44.6 mg, 61.0 μmol). The reaction mixture was stirred and reacted for 12 hours at 100° C., added with water (20 mL), and extracted with ethyl acetate (20 mL×1). The organic phase was washed with saturated brine (20 mL×1), dried over anhydrous sodium sulfate, filtered, and concentrated under reduced pressure. The residue was separated by silica gel column chromatography (1/1, petroleum ether/ethyl acetate, R_{f}=0.30) to obtain compound 93-5. MS-ESI calculated for [M+Na]⁺ 740, found 740.

### Step 5

Compound 93-5 (350 mg, 0.488 mmol) was dissolved in ethyl acetate (10.0 mL), and hydrochloride ethyl acetate solution (4 mol/L, 10 mL) was added dropwise thereto at 25° C. The reaction mixture was stirred for 1 hour at 25° C., concentrated under reduced pressure, and separated by preparative high performance liquid chromatography (chromatographic column: Phenomenex Genimi NX C18 150*40 mm*5 μm; mobile phase: mobile phase A: hydrochloric acid aqueous solution with a volume fraction of 0.05%; mobile phase B: acetonitrile; B %: 15 to 45%, 10 minutes) to obtain the hydrochloride of compound 93. ¹H NMR (400 MHz, CD₃OD) δ=8.66 (s, 1H), 8.04 (d, J=7.2 Hz, 1H), 7.69 (s, 1H), 7.66-7.59 (m, 1H), 7.38 (d, J=10.4 Hz, 1H), 7.32 (dd, J=1.2, 10.4 Hz, 1H), 7.27 (dd, J=1.6, 8.0 Hz, 1H), 4.60 (s, 2H), 4.47-4.38 (m, 1H), 4.22-4.13 (m, 1H), 3.58-3.41 (m, 3H), 2.27-2.16 (m, 1H), 1.99-1.90 (m, 1H), 1.86-1.68 (m, 2H), 0.98-0.92 (m, 2H), 0.91-0.86 (m, 2H). MS-ESI calculated for [M+H]⁺ 534, found 534.

### Example 94

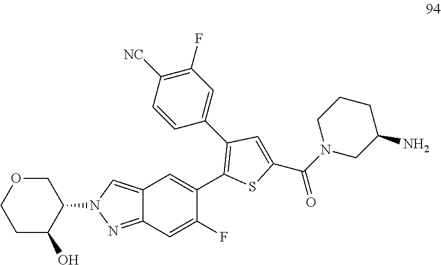 [SMILES: N#Cc1ccc(-c2cc(C(=O)N3CCC[C@@H](N)C3)sc2-c2cc3cn([C@H]4COCC[C@@H]4O)nc3cc2F)cc1F]

Synthetic Route:

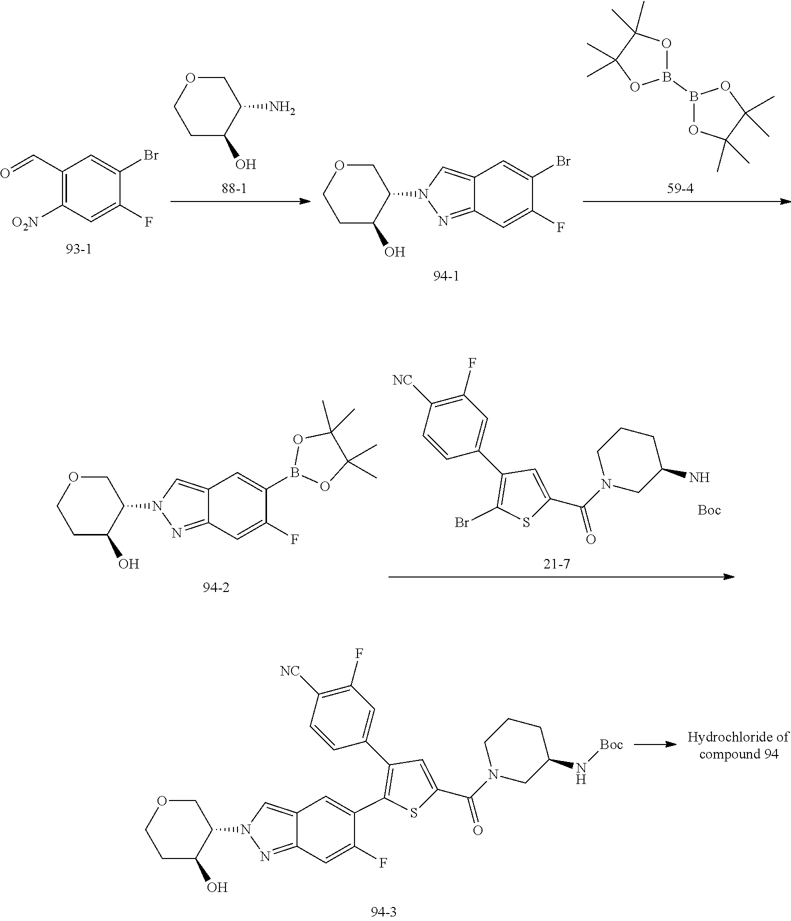

### Step 1

Under nitrogen atmosphere, compound 93-1 (500 mg, 2.02 mmol) was dissolved in isopropanol (15.0 mL), and compound 88-1 (272 mg, 2.32 mmol) was added thereto, and the reaction mixture was stirred for 4 hours at 80° C., added with tributylphosphine (1.22 g, 6.05 mmol) at 0° C., stirred for 12 hours at 80° C., and added with saturated ammonium chloride (30 mL), extracted with ethyl acetate (30 mL×1). The organic phase was washed with saturated brine (30 mL×1), dried over anhydrous sodium sulfate, filtered, and concentrated under reduced pressure. The residue was purified by silica gel column chromatography (1/1, petroleum ether/ethyl acetate, R_{f}=0.20) to obtain compound 94-1. MS-ESI calculated for [M+H]⁺ 315, found 315.

### Step 2

Under nitrogen atmosphere, compound 94-1 (460 mg, 1.46 mmol), bis(pinacolato)diboron (1.11 g, 4.38 mmol), and potassium acetate (430 mg, 4.38 mmol) were dissolved in dioxane (10.0 mL), and the reaction mixture was added with [1,1′-bis(diphenylphosphino)ferrocene]dichloropalladium(II) (107 mg, 0.146 mmol), and the reaction mixture was stirred and reacted for 12 hours at 95° C., and concentrated under reduced pressure. The residue was purified by silica gel column chromatography (1/1, petroleum ether/ethyl acetate, R_{f}=0.60) to obtain compound 94-2. MS-ESI calculated for [M+H]⁺ 363, found 363.

### Step 3

Under nitrogen atmosphere, compound 21-7 (320 mg, 0.629 mmol), compound 94-2 (402 mg, 0.944 mmol), and sodium carbonate (200 mg, 1.89 mmol) were dissolved in dioxane (8.00 mL) and water (2.00 mL), and the reaction mixture was added with [1,1′-bis(di-tert-butylphosphino)ferrocene]dichloropalladium(II) (46.1 mg, 63.0 μmol). The reaction mixture was stirred and reacted for 12 hours at 100° C., added with water (20 mL), and extracted with ethyl acetate (20 mL×1). The organic phase was washed with saturated brine (20 mL×1), dried over anhydrous sodium sulfate, filtered, and concentrated under reduced pressure. The residue was purified by silica gel column chromatography (1/1, petroleum ether/ethyl acetate, R_{f}=0.30) to obtain compound 94-3. MS-ESI calculated for [M+H]⁺ 664, found 664.

### Step 4

Compound 94-3 (230 mg, 0.347 mmol) was dissolved in ethyl acetate (10.0 mL), and hydrochloride ethyl acetate solution (4 mol/L, 10 mL) was added dropwise thereto at 25° C. The reaction mixture was stirred for 1 hour at 25° C., concentrated under reduced pressure, and separated by preparative high performance liquid chromatography (chromatographic column: Phenomenex Genimi NX C18 150*40 mm*5 μm; mobile phase: mobile phase A: hydrochloric acid aqueous solution with a volume fraction of 0.05%; mobile phase B: acetonitrile; B %: 10 to 40%, 10 minutes) to obtain the hydrochloride of compound 94. ¹H NMR (400 MHz, CD₃OD) δ=8.56 (s, 1H), 7.95 (d, J=6.8 Hz, 1H), 7.68 (s, 1H), 7.65-7.59 (m, 1H), 7.31 (d, J=10.8 Hz, 2H), 7.26 (dd, J=1.6, 8.0 Hz, 1H), 4.48-4.38 (m, 2H), 4.36-4.27 (m, 1H), 4.22-4.13 (m, 2H), 4.10-4.01 (m, 1H), 3.88 (t, J=10.8 Hz, 1H), 3.67-3.58 (m, 1H), 3.57-3.40 (m, 3H), 2.27-2.18 (m, 1H), 2.16-2.07 (m, 1H), 1.98-1.88 (m, 1H), 1.87-1.68 (m, 3H). MS-ESI calculated for [M+H]⁺ 564, found 564.

### Example 95

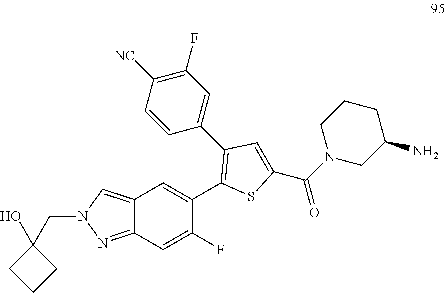 [SMILES: N#Cc1ccc(-c2cc(C(=O)N3CCC[C@@H](N)C3)sc2-c2cc3cn(CC4(O)CCC4)nc3cc2F)cc1F]

Synthetic Route:

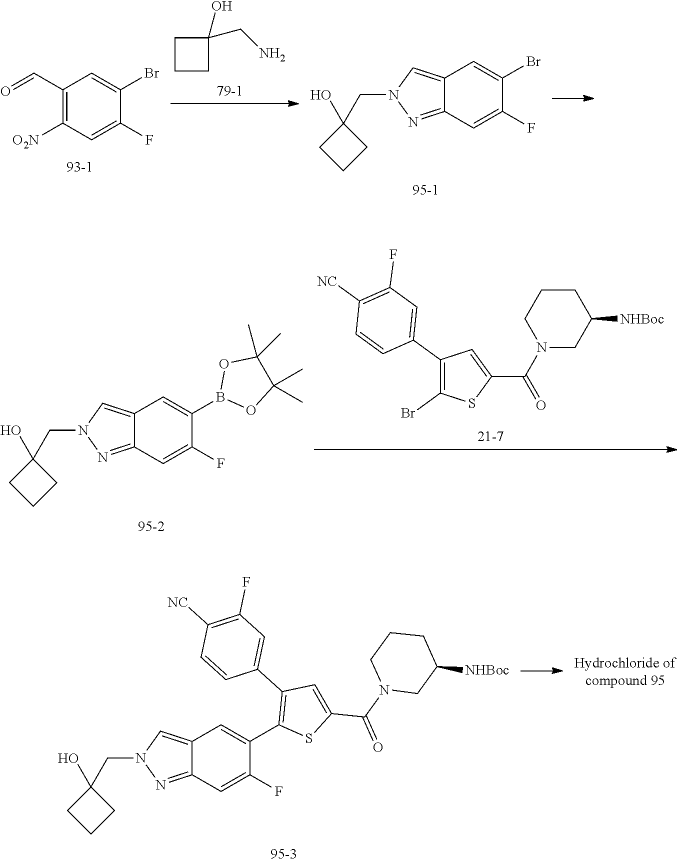

### Step 1

Compound 93-1 (1.50 g, 6.05 mmol) was dissolved in isopropanol (5.00 mL), then 79-1 (734 mg, 7.26 mmol) was added thereto, and the reaction mixture was stirred at 80° C. for 4 hours, then cooled to 25° C., added dropwise with tributylphosphine (4.48 mL, 18.1 mmol), stirred, and reacted for 12 hours at 80° C. The reaction mixture was added with water (50 mL), and extracted with ethyl acetate (50 mL×2). The organic phase was washed with saturated brine (50 mL×1), dried over anhydrous sodium sulfate, filtered, and concentrated under reduced pressure. The residue was purified by silica gel column chromatography (1/1, petroleum ether/ethyl acetate, R_{f}=0.43) to obtain compound 95-1. ¹H NMR (400 MHz, CD₃OD) δ=8.29 (s, 1H), 8.05 (d, J=6.4 Hz, 1H), 7.39 (d, J=9.6 Hz, 1H), 4.53 (s, 2H), 2.27-2.17 (m, 2H), 2.13-2.01 (m, 2H), 1.88-1.63 (m, 2H). MS-ESI calculated for [M+H]⁺ 299, found 299.

### Step 2

Under nitrogen atmosphere, compound 95-1 (700 mg, 2.34 mmol), bis(pinacolato)diboron (1.78 g, 7.02 mmol), and potassium acetate (689 mg, 7.02 mmol) were dissolved in dioxane (15.0 mL), and the reaction mixture was added with [1,1′-bis(diphenylphosphino)ferrocene]dichloropalladium(II) (171 mg, 234 μmol). The reaction mixture was stirred and reacted at 100° C. for 12 hours. The reaction mixture was concentrated under reduced pressure, and the residue was purified by silica gel column chromatography (1/1, petroleum ether/ethyl acetate, R_{f}=0.31) to obtain compound 95-2. MS-ESI calculated for [M+H]⁺ 347, found 347.

### Step 3

Under nitrogen atmosphere, compound 21-7 (330 mg, 650 μmol), compound 95-2 (450 mg, 1.30 mmol), and sodium carbonate (207 mg, 1.95 mmol) were dissolved in dioxane (24.0 mL) and water (6.00 mL), and the reaction mixture was added with [1,1′-bis(diphenylphosphino)ferrocene]dichloropalladium(II) (47.6 mg, 65.0 μmol), and the reaction mixture was stirred and reacted for 12 hours at 100° C. The reaction mixture was concentrated under reduced pressure, and the residue was purified by silica gel column chromatography (1/1, petroleum ether/ethyl acetate, R_{f}=0.22) to obtain compound 95-3. MS-ESI calculated for [M+H]⁺ 648, found 648.

### Step 4

Compound 95-3 (320 mg, 405 μmol) was dissolved in ethyl acetate (15.0 mL), and hydrochloride ethyl acetate solution (4 mol/L, 1.01 mL) was added thereto, and the reaction mixture was stirred and reacted for 1 hour at 25° C. After the reaction mixture was concentrated, the residue was purified by preparative high performance liquid chromatography (chromatographic column: Phenomenex Genimi NX C18 150*40 mm*5 μm, mobile phase A: hydrochloric acid aqueous solution with a volume fraction of 0.05%; mobile phase B: acetonitrile; B %: 15% to 45%, 10 minutes) to obtain the hydrochloride of compound 95. ¹H NMR (400 MHz, CD₃OD) δ=8.61 (s, 1H), 8.02 (d, J=7.2 Hz, 1H), 7.69 (s, 1H), 7.65-7.60 (m, 1H), 7.39-7.30 (m, 2H), 7.26 (d, J=8.0 Hz, 1H), 4.63 (s, 2H), 4.46-4.40 (m, 1H), 4.20-4.10 (m, 1H), 3.62-3.38 (m, 3H), 2.28-2.17 (m, 3H), 2.16-2.05 (m, 2H), 1.98-1.88 (m, 1H), 1.86-1.68 (m, 4H). MS-ESI calculated for [M+H]⁺ 548, found 548.

### Example 96

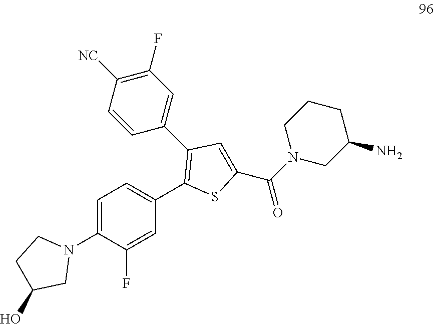 [SMILES: N#Cc1ccc(-c2cc(C(=O)N3CCC[C@@H](N)C3)sc2-c2ccc(N3CC[C@H](O)C3)c(F)c2)cc1F]

Synthetic Route:

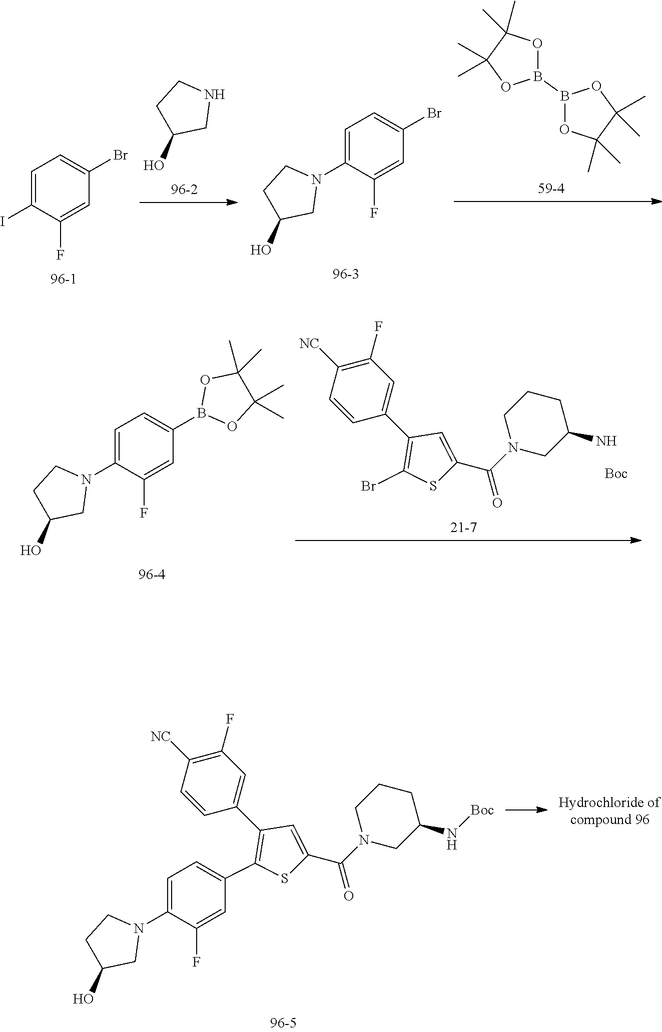

### Step 1

Under nitrogen atmosphere, compound 96-1 (2.6 g, 8.64 mmol) and compound 96-2 (1.51 g, 17.3 mmol) were dissolved in N,N-dimethylethanolamine (30.0 mL), and then potassium phosphate monohydrate (3.98 g, 17.3 mmol) and cuprous iodide (329 mg, 1.73 mmol) were added thereto, and the reaction mixture was stirred and reacted for 60 hours at 55° C. The reaction mixture was added with saturated ammonium chloride (50 mL), and extracted with dichloromethane (50 mL×2). The organic phase was washed with saturated brine (50 mL), dried over anhydrous sodium sulfate, filtered, and concentrated under reduced pressure. The residue was purified by silica gel column chromatography (3/1, petroleum ether/ethyl acetate, R_{f}=0.54) to obtain compound 96-3. MS-ESI calculated for [M+H]⁺ 262, found 262.

### Step 2

Under nitrogen atmosphere, compound 96-3 (260 mg, 1.00 mmol), bis(pinacolato)diboron (762 mg, 3.00 mmol), and potassium acetate (294 mg, 3.00 mmol) were dissolved in dioxane (10.0 mL), and the reaction mixture was added with [1,1′-bis(diphenylphosphino)ferrocene]dichloropalladium(II) (73.0 mg, 0.100 mmol), and the reaction mixture was stirred and reacted for 12 hours at 95° C., and concentrated under reduced pressure. The residue was purified by silica gel column chromatography (1/1, petroleum ether/ethyl acetate, R_{f}=0.61) to obtain compound 96-4. MS-ESI calculated for [M+H]⁺ 308, found 308.

### Step 3

Under nitrogen atmosphere, compound 21-7 (280 mg, 0.551 mmol), compound 96-4 (254 mg, 0.826 mmol), and sodium carbonate (175 mg, 1.65 mmol) were dissolved in dioxane (8.00 mL) and water (2.00 mL), and the reaction mixture was added with [1,1′-bis(di-tert-butylphosphino)ferrocene]dichloropalladium(II) (35.9 mg, 55.1 μmol). The reaction mixture was stirred and reacted for 12 hours at 100° C., added with water (20 mL), and extracted with ethyl acetate (20 mL×1). The organic phase was washed with saturated brine (20 mL×1), dried over anhydrous sodium sulfate, filtered, and concentrated under reduced pressure. The residue was purified by silica gel column chromatography (1/1, petroleum ether/ethyl acetate, R_{f}=0.36) to obtain compound 96-5. MS-ESI calculated for [M+H]⁺ 609, found 609.

### Step 4

Compound 96-5 (300 mg, 0.493 mmol) was dissolved in ethyl acetate (10.0 mL), and hydrochloride ethyl acetate solution (4 mol/L, 10 mL) was added dropwise thereto at 25° C. The reaction mixture was stirred for 1 hour at 25° C., concentrated under reduced pressure, and separated by preparative high performance liquid chromatography (chromatographic column: Phenomenex Genimi NX C18 150*40 mm*5 μm; mobile phase: mobile phase A: hydrochloric acid aqueous solution with a volume fraction of 0.05%; mobile phase B: acetonitrile; B %: 10 to 40%, 10 minutes) to obtain compound 96. ¹H NMR (400 MHz, CD₃OD) δ=7.70 (t, J=7.6 Hz, 1H), 7.55 (s, 1H), 7.37-7.27 (m, 2H), 7.08-6.94 (m, 3H), 4.60-4.52 (m, 1H), 4.47-4.36 (m, 1H), 4.21-4.10 (m, 1H), 3.80-3.67 (m, 2H), 3.64-3.55 (m, 1H), 3.53-3.38 (m, 4H), 2.25-2.13 (m, 2H), 2.11-2.01 (m, 1H), 1.97-1.87 (m, 1H), 1.85-1.66 (m, 2H). MS-ESI calculated for [M+H]⁺ 509, found 509.

### Example 97

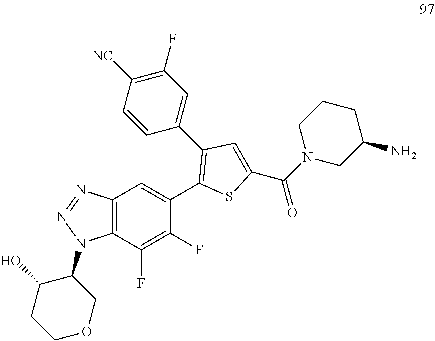 [SMILES: N#Cc1ccc(-c2cc(C(=O)N3CCC[C@@H](N)C3)sc2-c2cc3nnn([C@H]4COCC[C@@H]4O)c3c(F)c2F)cc1F]

Synthetic Route:

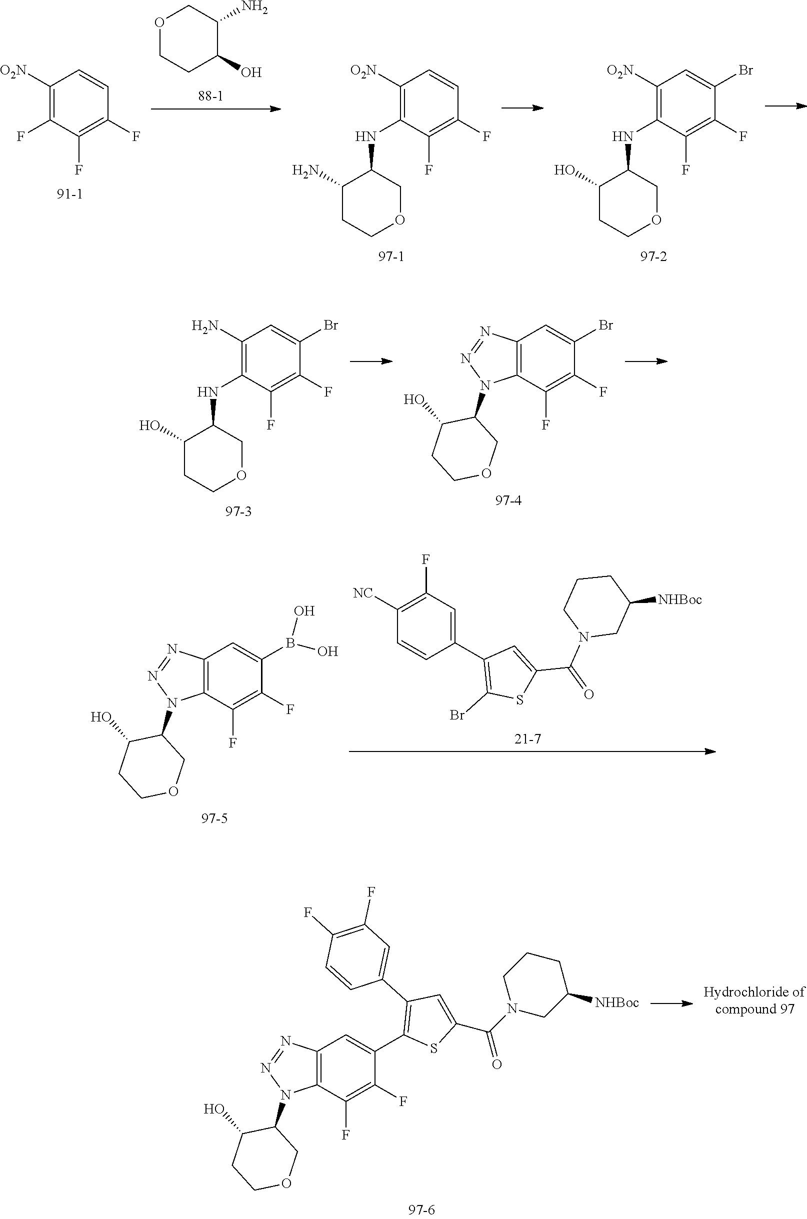

### Step 1

Compounds 91-1 (1.10 g, 6.21 mmol) and 88-1 (800 mg, 6.83 mmol) were dissolved in N,N-dimethylformamide (25.0 mL), and N,N-diisopropylethylamine (2.41 g, 18.6 mmol) was added thereto, and the reaction mixture was stirred and reacted for 12 hours at 80° C. The reaction mixture was added with water (50 mL), and extracted with ethyl acetate (50 mL×2). The organic phase was washed with saturated brine (50 mL×1), dried over anhydrous sodium sulfate, filtered, and concentrated under reduced pressure. The residue was purified by silica gel column chromatography (1/1, petroleum ether/ethyl acetate, R_{f}=0.27) to obtain compound 97-1. ¹H NMR (400 MHz) δ=8.06-7.94 (m, 1H), 6.68-6.52 (m, 1H), 3.73 (d, J=3.2 Hz, 2H), 2.21-2.05 (m, 4H), 1.91-1.72 (m, 1H), 1.70-1.53 (m, 1H). MS-ESI calculated for [M+H]⁺ 275, found 275.

### Step 2

Compound 97-1 (1.20 g, 7.75 mmol) was dissolved in N,N-dimethylformamide (20.0 mL). N-Bromosuccinimide (857 mg, 4.81 mmol) was added thereto, and the reaction mixture was stirred and reacted for 1 hour at 90° C. The reaction mixture was added with water (100 mL), and extracted with ethyl acetate (100 mL×2). The organic phase was washed with saturated brine (100 mL×1), dried over anhydrous sodium sulfate, filtered, and concentrated under reduced pressure. The residue was purified by silica gel column chromatography (3/1, petroleum ether/ethyl acetate, R_{f}=0.63) to obtain compound 97-2. ¹H NMR (400 MHz, CD₃OD) δ=8.29-8.27 (m, 1H), 4.12-4.07 (m, 1H), 3.97-3.86 (m, 1H), 3.83-3.71 (m, 2H), 3.59-3.54 (m, 1H), 3.42-3.38 (m, 1H), 2.08-1.94 (m, 1H), 1.64-1.56 (m, 1H). MS-ESI calculated for [M+H]⁺ 355, found 355.

### Step 3

Compound 97-2 (650 mg, 1.84 mmol) was dissolved in concentrated hydrochloric acid (10.0 mL), then tin dichloride (2.49 g, 11.0 mmol) was added thereto, and the reaction mixture was stirred and reacted for 1 hour at 25° C. The reaction mixture was adjusted to pH=11 with sodium hydroxide aqueous solution (4 mol/L), and extracted with ethyl acetate (50 mL×2). The organic phase was washed with saturated brine (50 mL×1), then dried over anhydrous sodium sulfate, filtered, and concentrated under reduced pressure to obtain crude compound 97-3, which was used directly in the next reaction step. MS-ESI calculated for [M+H]⁺ 323, found 323.

### Step 4

Compound 97-3 (0.55 g, 1.70 mmol) was dissolved in acetic acid (10.0 mL), and a solution of sodium nitrite (176 mg, 2.55 mmol) in water (5.00 mL) was added dropwise thereto, and the reaction mixture was stirred for 1 hour at 25° C. The reaction mixture was added with water (50 mL), and extracted with ethyl acetate (50 mL×2). The organic phase was washed with saturated ammonium chloride aqueous solution (50 mL×3), dried over anhydrous sodium sulfate, filtered, and concentrated under reduced pressure. The residue was purified by silica gel column chromatography (1/1, petroleum ether/ethyl acetate, R_{f}=0.59) to obtain compound 97-4. MS-ESI calculated for [M+H]⁺ 334, found 334.

### Step 5

Under nitrogen atmosphere, compound 97-4 (0.45 g, 1.35 mmol), bis(pinacolato)diboron (1.03 g, 4.04 mmol), and potassium acetate (396 mg, 4.04 mmol) were dissolved in dioxane (20.0 mL), and the reaction mixture was added with [1,1′-bis(diphenylphosphino)ferrocene]dichloropalladium(II) (98.5 mg, 135 μmol). The reaction mixture was stirred and reacted at 100° C. for 12 hours. The reaction mixture was concentrated under reduced pressure, and the residue was purified by silica gel column chromatography (2/1, petroleum ether/ethyl acetate, R_{f}=0.38) to obtain compound 97-5. ¹H NMR (400 MHz, CD₃OD) δ=8.16-8.14 (m, 1H), 4.80-4.76 (m, 1H), 4.45-4.33 (m, 1H), 4.30-4.25 (m, 1H), 4.16-4.10 (m, 1H), 4.06-3.99 (m, 1H), 3.78-3.60 (m, 1H), 2.23-2.14 (m, 1H), 1.95-1.81 (m, 1H). MS-ESI calculated for [M+H]⁺ 300, found 300.

### Step 6

Under nitrogen atmosphere, compound 21-7 (320 mg, 630 μmol), compound 97-5 (360 mg, 944 μmol), and sodium carbonate (133 mg, 1.26 mmol) were dissolved in dioxane (24.0 mL) and water (6.00 mL), and the reaction mixture was added with [1,1′-bis(diphenylphosphino)ferrocene]dichloropalladium(II) (46.1 mg, 63.0 μmol), and the reaction mixture was stirred and reacted for 12 hours at 100° C. The reaction mixture was concentrated under reduced pressure, and the residue was purified by silica gel column chromatography (2/1, petroleum ether/ethyl acetate, R_{f}=0.22) to obtain compound 97-6. MS-ESI calculated for [M+H−56]⁺ 627, found 627.

### Step 7

Compound 97-6 (270 mg, 178 μmol) was dissolved in ethyl acetate (10.0 mL), and hydrochloride ethyl acetate solution (4 mol/L, 0.44 mL) was added thereto, and the reaction mixture was stirred and reacted for 3 hours at 25° C. After the reaction mixture was concentrated, the residue was purified by preparative high performance liquid chromatography (chromatographic column: Phenomenex Genimi NX C18 150*40 mm*5 μm, mobile phase A: hydrochloric acid aqueous solution with a volume fraction of 0.05%; mobile phase B: acetonitrile; B %: 10% to 40%, 10 minutes) to obtain the hydrochloride of compound 97. ¹H NMR (400 MHz, CD₃OD) δ=8.01 (d, J=4.4 Hz, 1H), 7.72 (s, 1H), 7.70-7.64 (m, 1H), 7.41 (d, J=10.4 Hz, 1H), 7.26 (d, J=8.0 Hz, 1H), 4.84-4.73 (m, 1H), 4.48-4.27 (m, 3H), 4.23-3.99 (m, 3H), 3.74-3.64 (m, 1H), 3.62-3.41 (m, 3H), 2.22-2.15 (m, 2H), 2.01-1.69 (m, 4H). MS-ESI calculated for [M+H]⁺ 583, found 583.

### Example 98

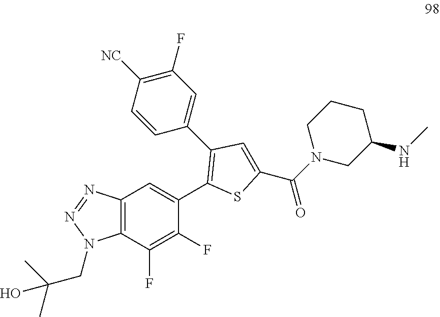 [SMILES: CN[C@@H]1CCCN(C(=O)c2cc(-c3ccc(C#N)c(F)c3)c(-c3cc4nnn(CC(C)(C)O)c4c(F)c3F)s2)C1]

Synthetic Route:

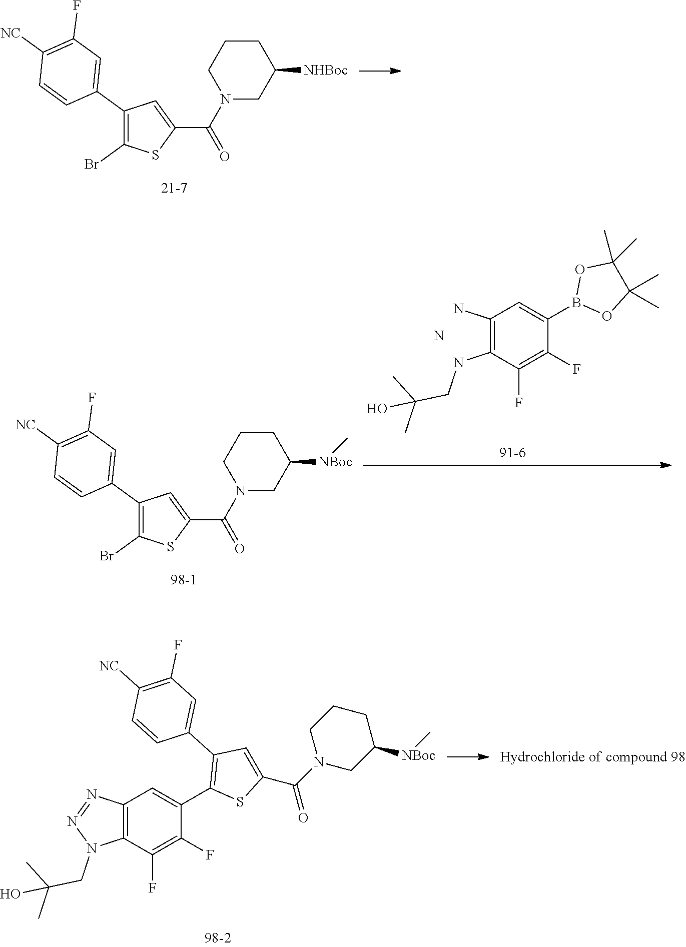

### Step 1

At 0° C., sodium hydride (118 mg, 6.52 mmol, purity: 60%) was dissolved in N,N-dimethylformamide (10.0 mL), and then the reaction mixture was added dropwise with a solution of 21-7 (500 mg, 983 μmol) in N,N-dimethylformamide (10.0 mL), and then added dropwise with iodomethane (306 μL, 4.92 mmol), stirred, and reacted for 12 hours at 25° C. The reaction mixture was added with saturated sodium bicarbonate aqueous solution (50 mL), and extracted with ethyl acetate (50 mL×2). The organic phase was washed with saturated brine (50 mL×1), dried over anhydrous sodium sulfate, filtered, and concentrated under reduced pressure. The residue was purified by silica gel column chromatography (2/1, petroleum ether/ethyl acetate, R_{f}=0.35) to obtain compound 98-1. MS-ESI calculated for [M+Na]⁺ 544, found 544.

### Step 2

Under nitrogen atmosphere, compound 98-1 (300 mg, 574 μmol), compound 91-6 (243 mg, 689 μmol), and sodium carbonate (183 mg, 1.72 mmol) were dissolved in dioxane (24.0 mL) and water (6.00 mL), and the reaction mixture was added with [1,1′-bis(diphenylphosphino)ferrocene]dichloropalladium(II) (42.0 mg, 57.4 μmol), and the reaction mixture was stirred and reacted for 12 hours at 100° C. The reaction mixture was concentrated under reduced pressure, and the residue was purified by silica gel column chromatography (1/1, petroleum ether/ethyl acetate, R_{f}=0.18) to obtain compound 98-2. MS-ESI calculated for [M+H]⁺ 669, found 669.

### Step 3

Compound 98-2 (110 mg, 164 μmol) was dissolved in ethyl acetate (5.00 mL), and hydrochloride ethyl acetate solution (4 mol/L, 0.82 mL) was added thereto, and the reaction mixture was stirred and reacted for 1 hour at 25° C. After the reaction mixture was concentrated, the residue was purified by preparative high performance liquid chromatography (chromatographic column: Phenomenex Genimi NX C18 150*30 mm*4 μm, mobile phase A: hydrochloric acid aqueous solution with a volume fraction of 0.05%; mobile phase B: acetonitrile; B %: 28% to 58%, 9 minutes) to obtain the hydrochloride of compound 98. ¹H NMR (400 MHz, CD₃OD) δ=8.00 (d, J=4.8 Hz, 1H), 7.74 (s, 1H), 7.67 (t, J=7.6 Hz, 1H), 7.40 (d, J=10.4 Hz, 1H), 7.26 (d, J=7.6 Hz, 1H), 4.76 (s, 2H), 4.32-4.28 (m, 1H), 4.15-4.10 (m, 1H), 3.82-3.70 (m, 2H), 3.45-3.99 (m, 1H), 2.83 (s, 3H), 2.32-2.27 (m, 1H), 1.99-1.88 (m, 2H), 1.80-1.70 (m, 1H), 1.31 (s, 6H). MS-ESI calculated for [M+H]⁺ 569, found 569.

### Example 99

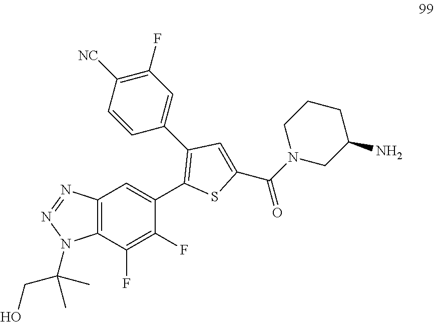 [SMILES: CC(C)(CO)n1nnc2cc(-c3sc(C(=O)N4CCC[C@@H](N)C4)cc3-c3ccc(C#N)c(F)c3)c(F)c(F)c21]

Synthetic Route:

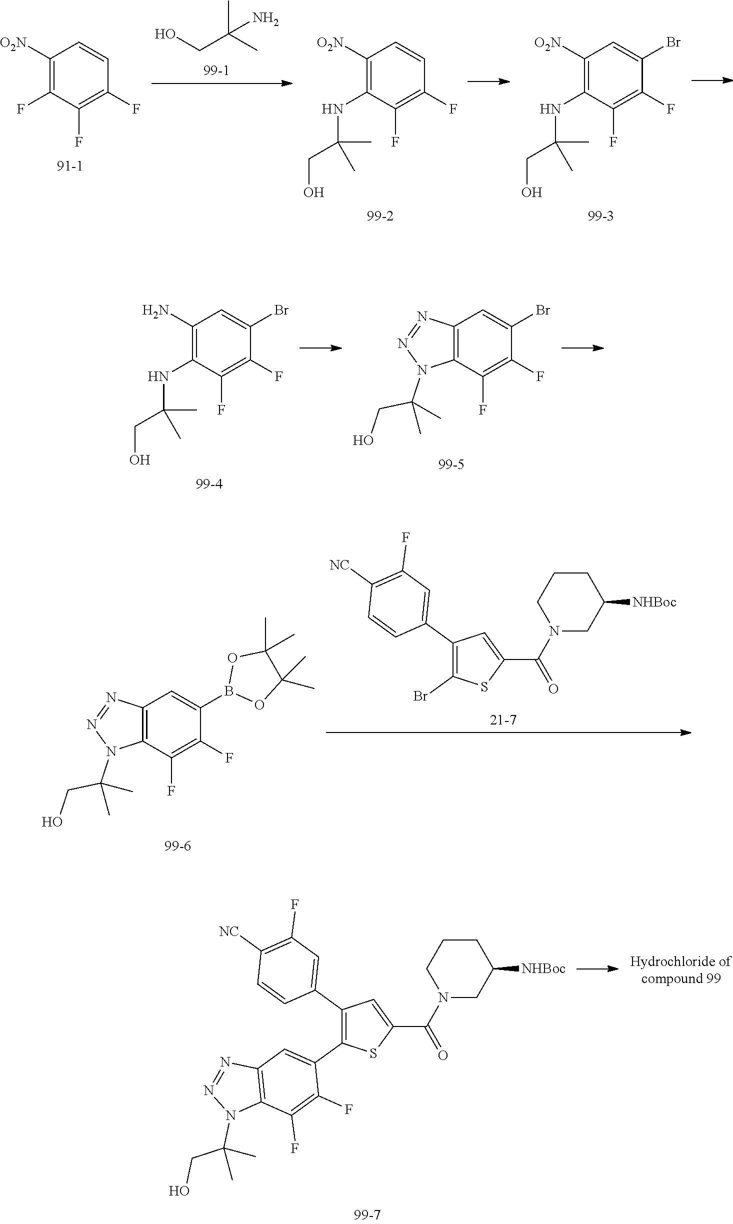

### Step 1

Compounds 91-1 (1.50 g, 8.47 mmol) and 99-1 (0.906 g, 10.2 mmol) were dissolved in N,N-dimethylformamide (25.0 mL), and N,N-diisopropylethylamine (3.28 g, 25.4 mmol) was added thereto, and the reaction mixture was stirred and reacted for 12 hours at 80° C. The reaction mixture was added with water (100 mL), and extracted with ethyl acetate (100 mL×2). The organic phase was washed with saturated brine (100 mL×1), dried over anhydrous sodium sulfate, filtered, and concentrated under reduced pressure. The residue was purified by silica gel column chromatography (2/1, petroleum ether/ethyl acetate, R_{f}=0.41) to obtain compound 99-2. ¹H NMR (400 MHz, CD₃OD) δ=8.04-8.01 (m, 1H), 6.85-6.68 (m, 1H), 3.55 (s, 2H), 1.35 (s, 3H), 1.34 (s, 3H). MS-ESI calculated for [M+H]⁺ 247, found 247.

### Step 2

Compound 99-2 (1.78 g, 7.23 mmol) was dissolved in N,N-dimethylformamide (30.0 mL). N-Bromosuccinimide (1.42 g, 7.95 mmol) was added thereto, and the reaction mixture was stirred and reacted for 1 hour at 90° C. The reaction mixture was added with water (50 mL), and extracted with ethyl acetate (50 mL×2). The organic phase was washed with saturated brine (50 mL×1), dried over anhydrous sodium sulfate, filtered, and concentrated under reduced pressure. The residue was purified by silica gel column chromatography (2/1, petroleum ether/ethyl acetate, R_{f}=0.48) to obtain compound 99-3. ¹H NMR (400 MHz, CD₃OD) δ=8.27-8.23 (m, 1H), 3.54 (s, 2H), 1.36 (s, 3H), 1.35 (s, 3H). MS-ESI calculated for [M+H]⁺ 325, found 325.

### Step 3

Compound 99-3 (2.00 g, 6.15 mmol) was dissolved in concentrated hydrochloric acid (20.0 mL), then tin dichloride (8.33 g, 36.9 mmol) was added thereto, and the reaction mixture was stirred and reacted for 1.5 hours at 25° C. The reaction mixture was adjusted to pH=11 with sodium hydroxide aqueous solution (4 mol/L), and extracted with ethyl acetate (100 mL×2). The organic phase was washed with saturated brine (100 mL×1), dried over anhydrous sodium sulfate, filtered, and concentrated under reduced pressure to obtain crude compound 99-4, which was used directly in the next reaction step. MS-ESI calculated for [M+H]⁺ 297, found 297.

### Step 4

Compound 99-4 (1.70 g, 5.76 mmol) was dissolved in acetic acid (20.0 mL), and a solution of sodium nitrite (596 mg, 8.64 mmol) in water (10.0 mL) was added dropwise thereto, and the reaction mixture was stirred for 2 hours at 25° C. The reaction mixture was added with water (100 mL), and extracted with ethyl acetate (100 mL×2). The organic phase was washed with saturated ammonium chloride aqueous solution (100 mL), dried over anhydrous sodium sulfate, filtered, and concentrated under reduced pressure. The residue was purified by silica gel column chromatography (2/1, petroleum ether/ethyl acetate, R_{f}=0.59) to obtain compound 99-5. ¹H NMR (400 MHz, CD₃OD) δ=8.20-8.19 (m, 1H), 4.02 (s, 2H), 1.85 (s, 6H). MS-ESI calculated for [M+H]⁺ 306, found 306.

### Step 5

Under nitrogen atmosphere, compound 99-5 (1.30 g, 4.25 mmol), bis(pinacolato)diboron (3.24 g, 12.7 mmol), and potassium acetate (1.25 g, 12.7 mmol) were dissolved in dioxane (30.0 mL), and the reaction mixture was added with [1,1′-bis(diphenylphosphino)ferrocene]dichloropalladium(II) (311 mg, 425 μmol). The reaction mixture was stirred and reacted at 100° C. for 12 hours. The reaction mixture was concentrated under reduced pressure, and the residue was purified by silica gel column chromatography (2/1, petroleum ether/ethyl acetate, R_{f}=0.44) to obtain compound 99-6. MS-ESI calculated for [M+H]⁺ 354 and 272, found 354 and 272.

### Step 6

Under nitrogen atmosphere, compound 21-7 (1.33 g, 2.61 mmol), compound 99-6 (1.66 g, 4.70 mmol), and sodium carbonate (830 mg, 7.83 mmol) were dissolved in dioxane (32.0 mL) and water (8.00 mL), and the reaction mixture was added with [1,1′-bis(diphenylphosphino)ferrocene]dichloropalladium(II) (191 mg, 261 μmol), and the reaction mixture was stirred and reacted for 12 hours at 100° C. The reaction mixture was concentrated under reduced pressure, and the residue was purified by silica gel column chromatography (1/1, petroleum ether/ethyl acetate, R_{f}=0.14) to obtain compound 99-7. MS-ESI calculated for [M+H]⁺ 655, found 655.

### Step 7

Compound 99-7 (400 mg, 397 μmol) was dissolved in ethyl acetate (10.0 mL), and hydrochloride ethyl acetate solution (4 mol/L, 0.99 mL) was added thereto, and the reaction mixture was stirred and reacted for 1 hour at 25° C. After the reaction mixture was concentrated, the residue was purified by preparative high performance liquid chromatography (chromatographic column: Phenomenex Genimi NX C18 150*40 mm*5 μm, mobile phase A: hydrochloric acid aqueous solution with a volume fraction of 0.05%; mobile phase B: acetonitrile; B %: 12% to 42%, 10 minutes) to obtain the hydrochloride of compound 99. ¹H NMR (400 MHz, CD₃OD) δ=8.02 (d, J=5.6 Hz, 1H), 7.72 (s, 1H), 7.70-7.65 (m, 1H), 7.43 (d, 10.4 Hz, 1H), 7.26 (d, J=8.4 Hz, 1H), 4.44-4.40 (m, 1H), 4.23-4.15 (m, 1H), 4.02 (s, 2H), 3.66-3.42 (m, 3H), 2.30-2.18 (m, 1H), 2.01-1.90 (m, 1H), 1.86 (s, 6H), 1.84-1.71 (m, 2H). MS-ESI calculated for [M+H]⁺ 555, found 555.

### Example 100

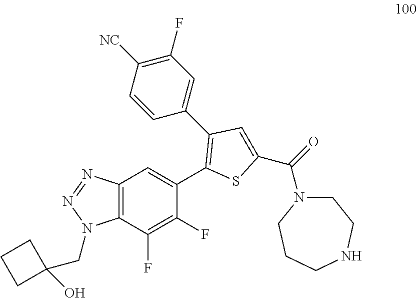 [SMILES: N#Cc1ccc(-c2cc(C(=O)N3CCCNCC3)sc2-c2cc3nnn(CC4(O)CCC4)c3c(F)c2F)cc1F]

Synthetic Route:

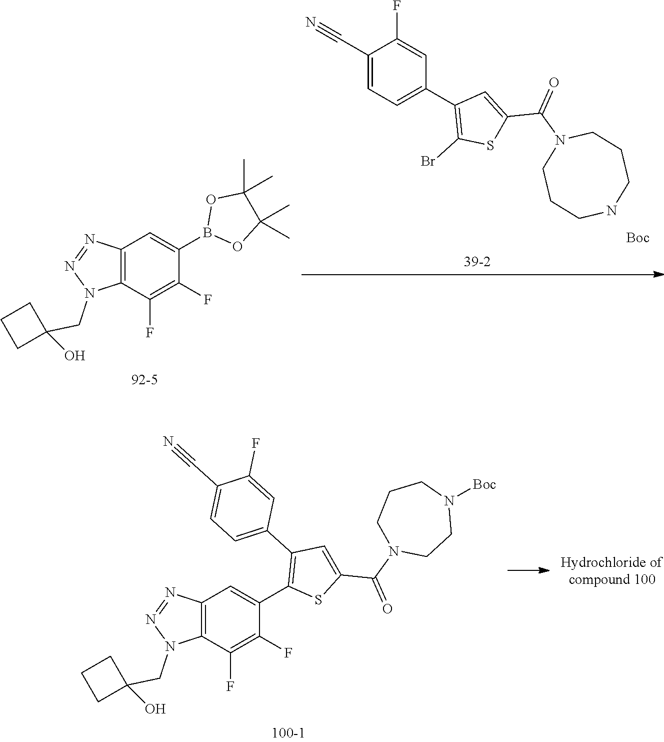 [SMILES: CC1(C)OB(c2cc3nnn(CC4(O)CCC4)c3c(F)c2F)OC1(C)C ; N#Cc1ccc(-c2cc(C(=O)N3CCCNCCC3)sc2Br)cc1F ; CC(C)(C)OC(=O)N1CCCN(C(=O)c2cc(-c3ccc(C#N)c(F)c3)c(-c3cc4nnn(CC5(O)CCC5)c4c(F)c3F)s2)CC1 ; "Boc"]

### Step 1

Under nitrogen atmosphere, compound 39-2 (300 mg, 0.590 mmol), compound 92-5 (259 mg, 0.708 mmol), and sodium carbonate (188 mg, 1.77 mmol) were dissolved in dioxane (8 mL) and water (2 mL), and the reaction mixture was added with [1,1′-bis(diphenylphosphino)ferrocene]dichloropalladium(II) (43.2 mg, 0.0590 mmol). The reaction mixture was stirred and reacted for 12 hours at 100° C., added with water (20 mL), and extracted with ethyl acetate (20 mL×1). The organic phase was washed with saturated brine (20 mL×1), dried over anhydrous sodium sulfate, filtered, and concentrated under reduced pressure. The residue was purified by silica gel column chromatography (1/1, petroleum ether/ethyl acetate, R_{f}=0.30) to obtain compound 100-1. MS-ESI calculated for [M−56+H]⁺ 611, found 611.

### Step 2

Compound 100-1 (100 mg, 0.150 mmol) was dissolved in ethyl acetate (3.5 mL), and hydrochloride ethyl acetate solution (4 mol/L, 3.5 mL) was added dropwise thereto. The reaction mixture was stirred for 1 hour at 25° C., concentrated under reduced pressure, and purified by preparative high performance liquid chromatography (chromatographic column: Phenomenex Synergi C18 150*30 mm*4 μm; mobile phase: mobile phase A: hydrochloric acid aqueous solution with a volume fraction of 0.05%; mobile phase B: acetonitrile; B %: 29% to 59%, 9 minutes) to obtain the hydrochloride of compound 100. ¹H NMR (400 MHz, CD₃OD) δ=7.95 (d, J=4.8 Hz, 1H), 7.74 (s, 1H), 7.64 (t, J=7.6 Hz, 1H), 7.37 (d, J=10.0 Hz, 1H), 7.22 (d, J=7.6 Hz, 1H), 4.89 (s, 2H), 4.17-3.90 (m, 4H), 3.54-3.44 (m, 2H), 3.39-3.40 (m, 2H), 2.39-2.31 (m, 2H), 2.30-2.20 (m, 2H), 2.13-2.02 (m, 2H), 1.92-1.81 (m, 1H), 1.80-1.70 (m, 1H). MS-ESI calculated for [M+H]⁺ 567, found 567.

### Example 101

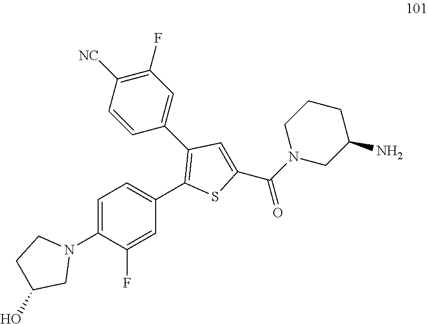 [SMILES: N#Cc1ccc(-c2cc(C(=O)N3CCC[C@@H](N)C3)sc2-c2ccc(N3CC[C@@H](O)C3)c(F)c2)cc1F]

Synthetic Route:

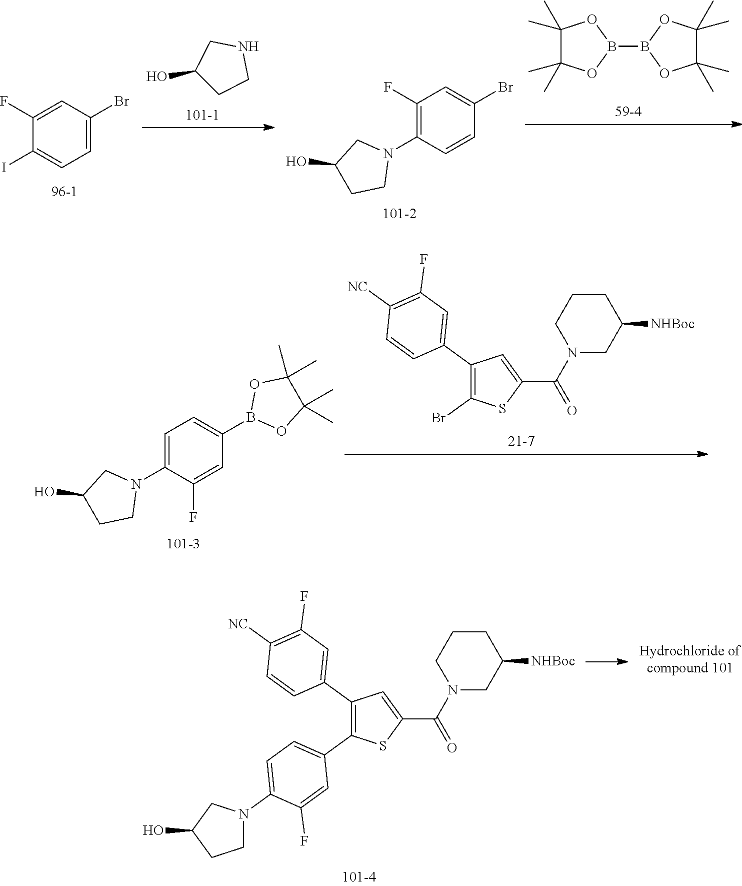

### Step 1

Compounds 96-1 (3.10 g, 10.30 mmol) and 101-1 (1.80 g, 20.61 mmol) were dissolved in N,N-dimethylethanolamine (20.0 mL), and then potassium phosphate monohydrate (4.74 g, 20.61 mmol) and cuprous iodide (392 mg, 2.06 mmol) were added thereto. The reaction mixture was stirred and reacted for 60 hours at 55° C. The reaction mixture was added with saturated ammonium chloride solution (80 mL), and extracted with dichloromethane (80 mL×2). The organic phase was washed with saturated brine (80 mL×1), dried over anhydrous sodium sulfate, filtered, and concentrated under reduced pressure. The residue was purified by silica gel column chromatography (3/1, petroleum ether/ethyl acetate, R_{f}=0.36) to obtain compound 101-2. MS-ESI calculated for [M+H]⁺ 260, found 260.

### Step 2

Under nitrogen atmosphere, compounds 101-2 (390 mg, 1.50 mmol), 59-4 (1.14 g, 4.50 mmol), and potassium acetate (441 mg, 4.50 mmol) were dissolved in dioxane (10.0 mL), and the reaction mixture was added with [1,1′-bis(diphenylphosphino)ferrocene]dichloropalladium(II) (109 mg, 150 μmol). The reaction mixture was stirred and reacted at 100° C. for 12 hours. The reaction mixture was concentrated under reduced pressure, and the residue was purified by silica gel column chromatography (3/1, petroleum ether/ethyl acetate, R_{f}=0.40) to obtain compound 101-3. MS-ESI calculated for [M+H]⁺ 308, found 308.

### Step 3

Under nitrogen atmosphere, compound 21-7 (450 mg, 885 μmol), compound 101-3 (326 mg, 1.06 mmol), and sodium carbonate (187 mg, 1.77 mmol) were dissolved in dioxane (10.0 mL) and water (2.00 mL), and the reaction mixture was added with [1,1′-bis(diphenylphosphino)ferrocene]dichloropalladium(II) (65.0 mg, 89.0 μmol), and the reaction mixture was stirred and reacted for 12 hours at 100° C. The reaction mixture was concentrated under reduced pressure, and the residue was purified by silica gel column chromatography (1/1, petroleum ether/ethyl acetate, R_{f}=0.18) to obtain compound 101-4. MS-ESI calculated for [M+H]⁺ 609, found 609.

### Step 4

Compound 101-4 (323 mg, 530 μmol) was dissolved in ethyl acetate (4.00 mL), and hydrochloride ethyl acetate solution (4 mol/L, 2.65 mL) was added thereto, and the reaction mixture was stirred and reacted for 1 hour at 25° C. After the reaction mixture was concentrated, the residue was purified by preparative high performance liquid chromatography (chromatographic column: Phenomenex C18 150*40 mm*5 μm, mobile phase A: hydrochloric acid aqueous solution with a volume fraction of 0.05%; mobile phase B: acetonitrile; B %: 10% to 50%, 10 minutes) to obtain the hydrochloride of compound 101. ¹H NMR (400 MHz, CD₃OD) δ=7.71 (t, J=7.2 Hz, 1H), 7.56 (s, 1H), 7.39-7.28 (m, 2H), 7.00 (d, J=12.0 Hz, 2H), 6.93-6.81 (m, 1H), 4.62-4.50 (m, 1H), 4.42 (d, J=12.0 Hz, 1H), 4.17 (d, J=13.6 Hz, 1H), 3.80-3.64 (m, 2H), 3.58-3.41 (m, 5H), 2.25-2.12 (m, 2H), 2.09-2.01 (m, 1H), 1.97-1.88 (m, 1H), 1.85-1.71 (m, 2H). MS-ESI calculated for [M+H]⁺ 509, found 509.

### Example 102

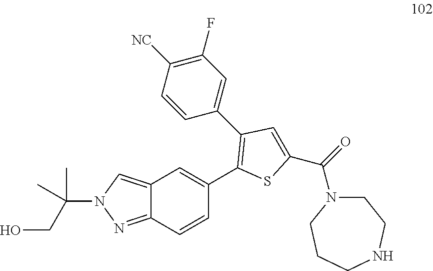 [SMILES: CC(C)(CO)n1cc2cc(-c3sc(C(=O)N4CCCNCC4)cc3-c3ccc(C#N)c(F)c3)ccc2n1]

Synthetic Route:

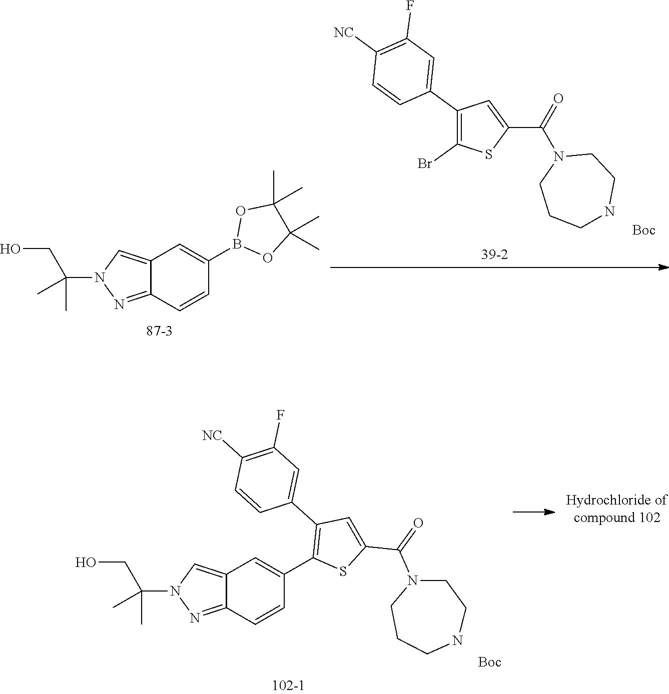

### Step 1

Under nitrogen atmosphere, compound 39-2 (500 mg, 0.983 mmol), compound 87-3 (373 mg, 1.18 mmol), and sodium carbonate (313 mg, 2.95 mmol) were dissolved in dioxane (12.0 mL) and water (3.00 mL), and the reaction mixture was added with [1,1′-bis(diphenylphosphino)ferrocene]dichloropalladium(II) (72.0 mg, 980 μmol). The reaction mixture was stirred and reacted for 12 hours at 100° C., added with water (20 mL), and extracted with ethyl acetate (20 mL×1). The organic phase was washed with saturated brine (20 mL×1), dried over anhydrous sodium sulfate, filtered, and concentrated under reduced pressure. The residue was purified by silica gel column chromatography (1/1, petroleum ether/ethyl acetate, R_{f}=0.30) to obtain compound 102-1. MS-ESI calculated for [M+H]⁺ 618, found 618.

### Step 2

Compound 102-1 (200 mg, 0.347 mmol) was dissolved in ethyl acetate (5.00 mL), and hydrochloride ethyl acetate solution (4 mol/L, 7.02 mL) was added thereto. The reaction mixture was stirred for 1 hour at 25° C., concentrated under reduced pressure, and purified by preparative high performance liquid chromatography (chromatographic column: Xtimate C18 150*40 mm*5 μm; mobile phase: mobile phase A: hydrochloric acid aqueous solution with a volume fraction of 0.05%; mobile phase B: acetonitrile; B %: 10 to 40%, 10 minutes) to obtain the hydrochloride of compound 102. ¹H NMR (400 MHz, CD₃OD) δ=8.77 (s, 1H), 7.93 (s, 1H), 7.73-7.59 (m, 3H), 7.42-7.21 (m, 3H), 4.30-4.25 (m, 4H), 3.89 (s, 2H), 3.56-3.44 (m, 2H), 3.42-3.36 (m, 2H), 2.36-2.18 (m, 2H), 1.78 (s, 6H). MS-ESI calculated for [M+H]⁺ 518, found 518.

### Example 103

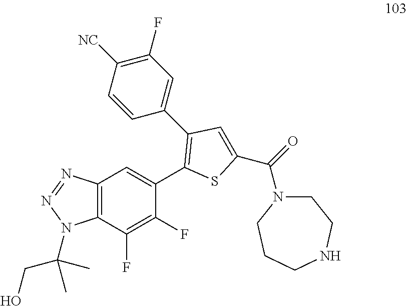 [SMILES: CC(C)(CO)n1nnc2cc(-c3sc(C(=O)N4CCCNCC4)cc3-c3ccc(C#N)c(F)c3)c(F)c(F)c21]

Synthetic Route:

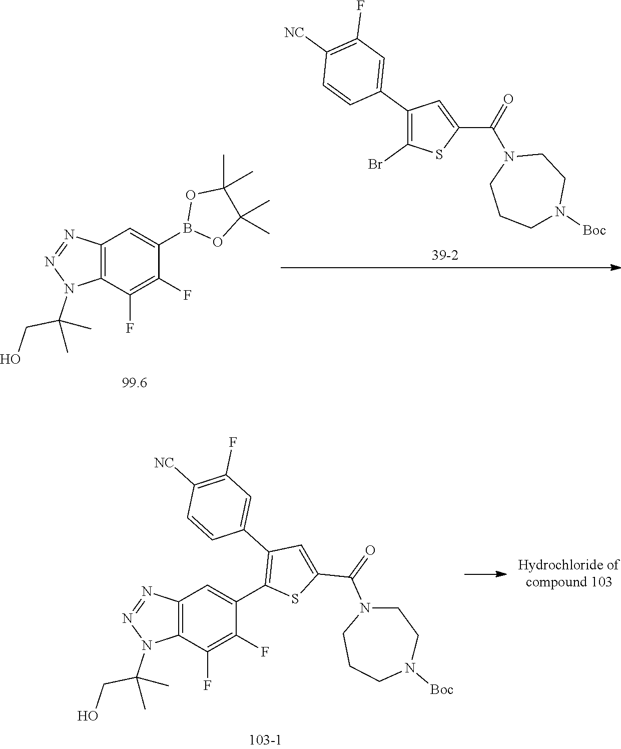 [SMILES: CC(C)(CO)n1nnc2cc(B3OC(C)(C)C(C)(C)O3)c(F)c(F)c21 ; CC(C)(C)OC(=O)N1CCCN(C(=O)c2cc(-c3ccc(C#N)c(F)c3)c(Br)s2)CC1 ; CC(C)(C)OC(=O)N1CCCN(C(=O)c2cc(-c3ccc(C#N)c(F)c3)c(-c3cc4nnn(C(C)(C)CO)c4c(F)c3F)s2)CC1]

### Step 1

Under nitrogen atmosphere, compound 39-2 (380 mg, 0.747 mmol), compound 99-6 (317 mg, 0.897 mmol), and potassium phosphate (476 mg, 2.24 mmol) were dissolved in dioxane (12.0 mL) and water (3.00 mL), and the reaction mixture was added with [1,1′-bis(di-tert-butylphosphino)ferrocene]dichloropalladium(II) (49.0 mg, 74.7 μmol). The reaction mixture was stirred and reacted for 12 hours at 100° C., added with water (20 mL), and extracted with ethyl acetate (20 mL×1). The organic phase was washed with saturated brine (20 mL×1), dried over anhydrous sodium sulfate, filtered, and concentrated under reduced pressure. The residue was purified by silica gel column chromatography (1/1, petroleum ether/ethyl acetate, R_{f}=0.30) to obtain compound 103-1. MS-ESI calculated for [M+H]⁺ 655, found 655.

### Step 2

Compound 103-1 (160 mg, 244 μmol) was dissolved in ethyl acetate (3.00 mL), and hydrochloride ethyl acetate solution (4 mol/L, 15.0 mL) was added thereto. The reaction mixture was stirred for 1 hour at 25° C., concentrated under reduced pressure, and purified by preparative high performance liquid chromatography (chromatographic column: Phenomenex C18 150*40 mm*5 μm; mobile phase: A: hydrochloric acid aqueous solution with a volume fraction of 0.05%; mobile phase B: acetonitrile 12% to 42%, 10 minutes) to obtain the hydrochloride of compound 103. ¹H NMR (400 MHz, CD₃OD) δ=7.90 (d, J=4.4 Hz, 1H), 7.65 (s, 1H), 7.56 (t, J=7.6 Hz, 1H), 7.31 (d, J=10.0 Hz, 1H), 7.14 (d, J=7.6 Hz, 1H), 4.20-3.85 (m, 6H), 3.48-3.35 (m, 2H), 3.21 (s, 2H), 2.25-2.10 (m, 2H), 1.74 (s, 6H). MS-ESI calculated for [M+H]⁺ 555, found 555.

### Example 104

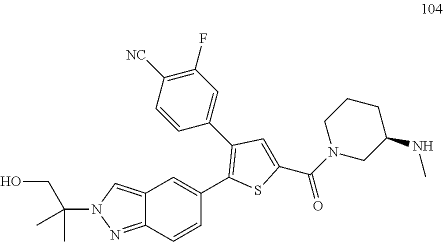 [SMILES: CN[C@@H]1CCCN(C(=O)c2cc(-c3ccc(C#N)c(F)c3)c(-c3ccc4nn(C(C)(C)CO)cc4c3)s2)C1]

Synthetic Route:

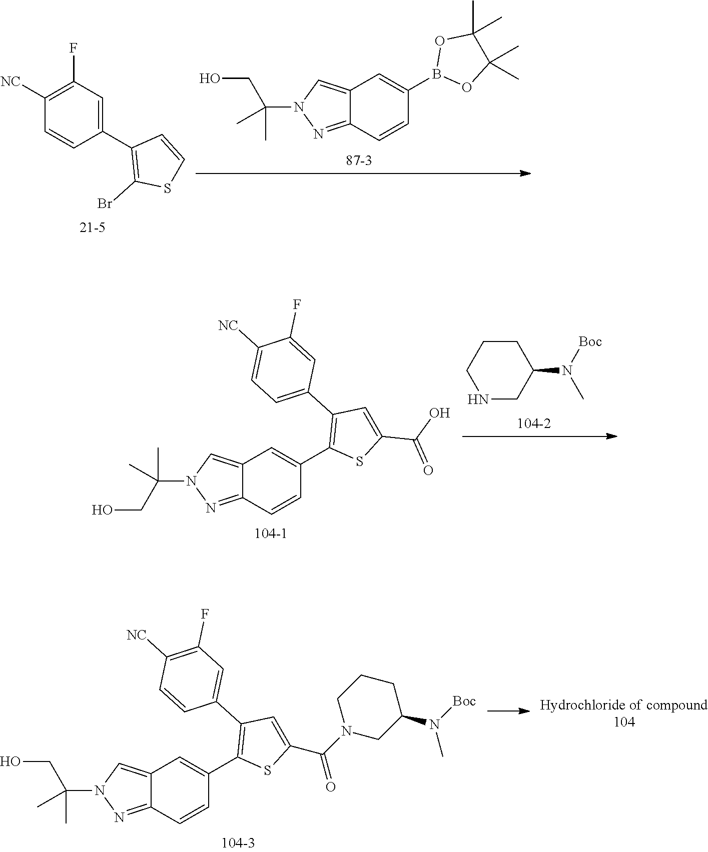 [SMILES: N#Cc1ccc(-c2ccsc2Br)cc1F ; CC(C)(CO)n1cc2cc(B3OC(C)(C)C(C)(C)O3)ccc2n1 ; CC(C)(CO)n1cc2cc(-c3sc(C(=O)O)cc3-c3ccc(C#N)c(F)c3)ccc2n1 ; CN(C(=O)OC(C)(C)C)[C@@H]1CCCNC1 ; CN(C(=O)OC(C)(C)C)[C@@H]1CCCN(C(=O)c2cc(-c3ccc(C#N)c(F)c3)c(-c3ccc4nn(C(C)(C)CO)cc4c3)s2)C1]

### Step 1

Under nitrogen atmosphere, compound 21-5 (4.69 g, 14.4 mmol), compound 87-3 (5.00 g, 15.8 mmol), and sodium carbonate (3.05 mg, 28.8 mmol) were dissolved in N,N-dimethylacetamide (40.0 mL) and water (10.0 mL), and the reaction mixture was added with [1,1′-bis(diphenylphosphino)ferrocene]dichloropalladium(II) (101 mg, 0.138 mmol), stirred, and reacted for 12 hours at 90° C., added with water (1000 mL), added with hydrochloric acid (1 mol/L) dropwise to adjust the pH to 3.0, and concentrated under reduced pressure to obtain compound 104-1. MS-ESI calculated for [M+H]⁺ 436, found 436.

### Step 2

Under nitrogen atmosphere, compound 104-1 (500 mg, 1.15 mmol) was dissolved in anhydrous dichloromethane (10.0 mL), and then triethylamine (349 mg, 3.44 mmol), compound 104-2 (295 mg, 1.38 mmol), and 2-(7-azabenzotriazol-1-yl)-N,N,N′,N′-tetramethyluronium hexafluorophosphate (655 mg, 1.72 mmol) were added thereto. The reaction mixture was stirred for 2 hours at 25° C. The reaction mixture was added with water (20 mL), and extracted with dichloromethane (20 mL×1). The organic phase was washed with saturated brine (20 mL×1), dried over anhydrous sodium sulfate, filtered, and concentrated under reduced pressure. The residue was purified by silica gel column chromatography (1/1, petroleum ether/ethyl acetate, R_{f}=0.21) to obtain compound 104-3. ¹H NMR (400 MHz, CDCl₃) δ 8.15-8.05 (m, 1H), 7.75-7.69 (m, 1H), 7.62 (d, J=8.8 Hz, 1H), 7.50-7.37 (m, 2H), 7.27-7.20 (m, 2H), 7.10 (d, J=8.4 Hz, 1H), 4.68-4.55 (m, 1H), 4.51-4.43 (m, 1H), 3.95 (s, 2H), 3.66-3.56 (m, 1H), 3.06-2.89 (m, 2H), 2.83 (s, 3H), 2.02-1.90 (m, 4H), 1.73 (s, 9H), 1.46 (s, 6H). MS-ESI calculated for [M+H]⁺ 632, found 632.

### Step 3

Compound 104-3 (600 mg, 0.950 mmol) was dissolved in ethyl acetate (10.0 mL), and hydrochloride ethyl acetate solution (4 mol/L, 5.00 mL) was added dropwise thereto at 25° C. The reaction mixture was stirred for 1 hour at 25° C., concentrated under reduced pressure, and purified by preparative high performance liquid chromatography (chromatographic column: Phenomenex Genimi NX C18 150*40 mm*5 μm; mobile phase: mobile phase A: hydrochloric acid aqueous solution with a volume fraction of 0.05%; mobile phase B: acetonitrile; B %: 15 to 45%, 10 minutes) to obtain the hydrochloride of compound 104. ¹H NMR (400 MHz, CD₃OD) δ 8.65 (s, 1H), 7.89 (s, 1H), 7.69-7.62 (m, 3H), 7.38-7.25 (m, 3H), 4.35-4.23 (m, 1H), 4.15-4.02 (m, 1H), 3.89 (s, 2H), 3.80-3.61 (m, 2H), 3.44-3.34 (m, 1H), 2.80 (s, 3H), 2.33-2.20 (m, 1H), 1.97-1.81 (m, 2H), 1.76 (s, 6H), 1.75-1.69 (m, 1H). MS-ESI calculated for [M+H]⁺ 532, found 532.

### Example 105

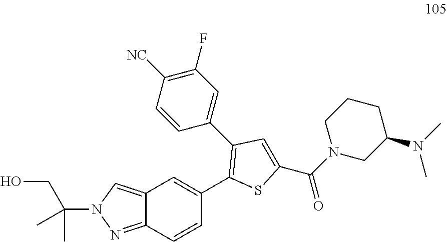 [SMILES: CN(C)[C@@H]1CCCN(C(=O)c2cc(-c3ccc(C#N)c(F)c3)c(-c3ccc4nn(C(C)(C)CO)cc4c3)s2)C1]

Synthetic Route:

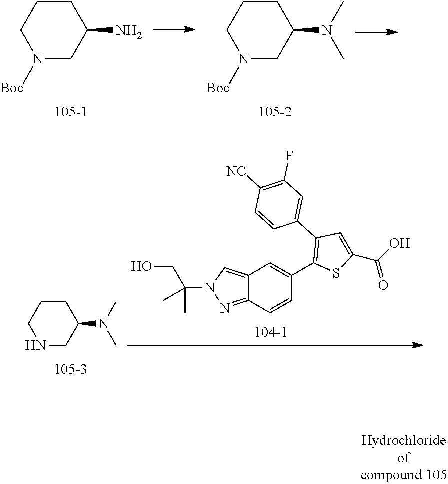

### Step 1

Under nitrogen atmosphere, compound 105-1 (1.0 g, 4.99 mmol) was dissolved in anhydrous dichloromethane (10.0 mL), and then 37% of formaldehyde aqueous solution (2.03 g, 25.0 mmol), acetic acid (30.0 mg, 0.499 mmol), and sodium triacetoxyborohydride (2.12 g, 9.99 mmol) were added thereto. The reaction mixture was stirred for 12 hours at 25° C., added with saturated sodium bicarbonate (20 mL), and extracted with dichloromethane (20 mL×1). The organic phase was washed with saturated brine (20 mL×1), dried over anhydrous sodium sulfate, filtered, and concentrated under reduced pressure to obtain compound 105-2. ¹H NMR (400 MHz, CDCl₃) δ 4.40-4.08 (m, 1H), 4.04-3.90 (m, 1H), 2.69-2.62 (m, 1H), 2.32 (s, 6H), 2.20-2.10 (m, 1H), 2.04-1.95 (m, 1H), 1.94-1.82 (m, 1H), 1.77-1.67 (m, 1H), 1.46 (s, 9H), 1.43-1.24 (m, 2H).

### Step 2

Compound 105-2 (1.1 g, 4.82 mmol) was dissolved in ethyl acetate (10 mL), and hydrochloride ethyl acetate solution (4 mol/L, 10 mL) was added dropwise thereto at 25° C. The reaction mixture was stirred for 1 hour at 25° C., and concentrated under reduced pressure to obtain the hydrochloride of compound 105-3. ¹H NMR (400 MHz, CD₃OD) δ 3.84-3.76 (m, 1H), 3.72-3.63 (m, 1H), 3.46-3.39 (m, 1H), 3.30-3.23 (m, 1H), 3.07-2.99 (m, 1H), 2.99-2.93 (m, 6H), 2.34-2.26 (m, 1H), 2.22-2.12 (m, 1H), 1.94-1.82 (m, 2H).

### Step 3

Under nitrogen atmosphere, compound 104-1 (350 mg, 1.15 mmol) was dissolved in anhydrous dichloromethane (10 mL), and then triethylamine (407 mg, 4.02 mmol), the hydrochloride of compound 105-3 (172 mg, 1.04 mmol), and 2-(7-azabenzotriazol-1-yl)-N,N,N′,N′-tetramethyluronium hexafluorophosphate (458 mg, 1.21 mmol) were added thereto, and the reaction mixture was stirred for 2 hours at 25° C. The reaction mixture was added with water (20 mL) and extracted with dichloromethane (20 mL×1). The organic phase was washed with saturated brine (20 mL×1), dried over anhydrous sodium sulfate, filtered, and concentrated under reduced pressure. The residue was purified by preparative high performance liquid chromatography (chromatographic column: Phenomenex Genimi NX C18 150*40 mm*5 μm; mobile phase: mobile phase A: hydrochloric acid aqueous solution with a volume fraction of 0.05%; mobile phase B: acetonitrile; B %: 15 to 45%, 10 minutes) to obtain the hydrochloride of compound 105. ¹H NMR (400 MHz, CD₃OD) δ 8.69 (s, 1H), 7.90 (s, 1H), 7.70-7.60 (m, 3H), 7.39-7.22 (m, 3H), 4.65-4.53 (m, 1H), 4.36-4.22 (m, 1H), 3.89 (s, 2H), 3.66-3.56 (m, 1H), 3.55-3.38 (m, 2H), 3.01 (s, 3H), 2.96 (s, 3H), 2.34-2.22 (m, 1H), 2.04-1.92 (m, 2H), 1.77 (s, 6H), 1.75-1.68 (m, 1H). MS-ESI calculated for [M+H]⁺ 546, found 546.

### Example 106

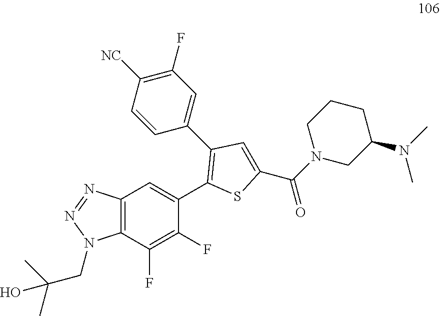 [SMILES: CN(C)[C@@H]1CCCN(C(=O)c2cc(-c3ccc(C#N)c(F)c3)c(-c3cc4nnn(CC(C)(C)O)c4c(F)c3F)s2)C1]

Synthetic Route:

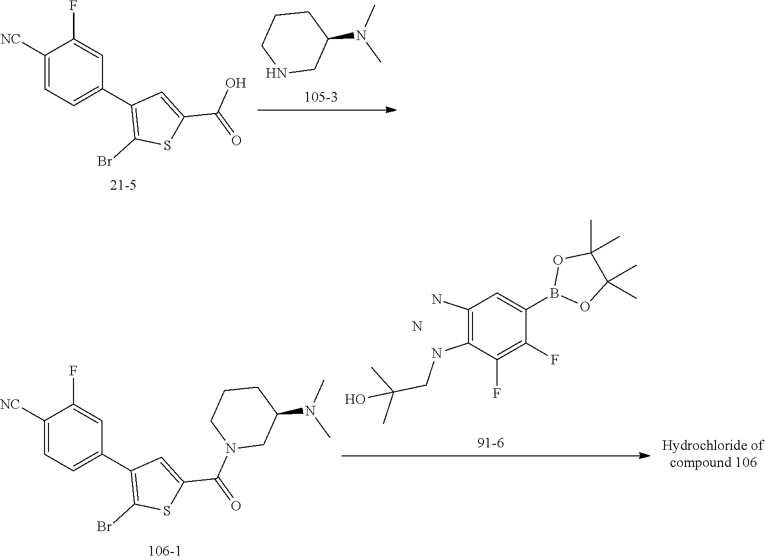

### Step 1

Compound 21-5 (600 mg, 1.84 mmol) and the hydrochloride of 105-3 (393 mg, 2.39 mmol) were dissolved in N,N-dimethylformamide (15.0 mL), and then 2-(7-azabenzotriazol-1-yl)-N,N,N′,N′-tetramethyluronium hexafluorophosphate (1.05 g, 2.76 mmol) and N,N-diisopropylethylamine (713 mg, 5.52 mmol) were added thereto, and the reaction mixture was stirred and reacted for 2 hours at 25° C. The reaction mixture was added with water (100 mL), and extracted with ethyl acetate (50 mL×2). The organic phase was washed with saturated brine (40 mL×3), dried over anhydrous sodium sulfate, filtered, and concentrated under reduced pressure. The residue was purified by silica gel column chromatography (1/1, petroleum ether/ethyl acetate, R_{f}=0.53) to obtain compound 106-1. ¹H NMR (400 MHz, CD₃OD) δ=7.90-7.84 (m, 1H), 7.67-7.61 (m, 2H), 7.47 (s, 1H), 4.68-4.53 (m, 1H), 4.43 (d, J=11.6 Hz, 1H), 4.31-4.14 (m, 1H), 3.16-3.12 (m, 1H), 2.58-2.56 (m, 1H), 2.42 (s, 6H), 2.10-2.08 (m, 1H), 1.89-1.86 (m, 1H), 1.66-1.56 (m, 2H). MS-ESI calculated for [M+H]⁺ 436, found 436.

### Step 2

Under nitrogen atmosphere, compound 106-1 (1.10 g, 2.03 mmol), compound 91-6 (1.07 g, 2.77 mmol), and potassium phosphate (956 mg, 4.06 mmol) were dissolved in dioxane (16.0 mL) and water (4.00 mL), and the reaction mixture was added with [1,1′-bis(di-tert-butylphosphino)ferrocene]dichloropalladium(II) (132 mg, 202 μmol), stirred, and reacted for 12 hours at 90° C. The reaction mixture was concentrated under reduced pressure, and the residue was purified by silica gel column chromatography (1/1, petroleum ether/ethyl acetate, R_{f}=0.43) and then purified by high performance liquid chromatography (chromatographic column: Xtimate C18 150*40 mm*5 μm, mobile phase A: hydrochloric acid aqueous solution with a volume fraction of 0.05%; mobile phase B: acetonitrile; B %: 15% to 45%, 10 minutes) to obtain the hydrochloride of compound 106. ¹H NMR (400 MHz, CD₃OD) δ=8.00 (d, J=4.8 Hz, 1H), 7.74 (s, 1H), 7.67 (t, J=7.6 Hz, 1H), 7.40 (d, J=9.6 Hz, 1H), 7.29-7.22 (m, 1H), 4.76 (s, 2H), 4.57 (d, J=12.4 Hz, 1H), 4.30-4.27 (m, 1H), 3.67-3.62 (m, 1H), 3.52-3.50 (m, 2H), 3.04-2.98 (m, 6H), 2.31-2.29 (m, 1H), 2.02-1.99 (m, 2H), 1.78-1.75 (m, 1H), 1.31 (s, 6H). MS-ESI calculated for [M+H]⁺ 583, found 583.

### Example 107

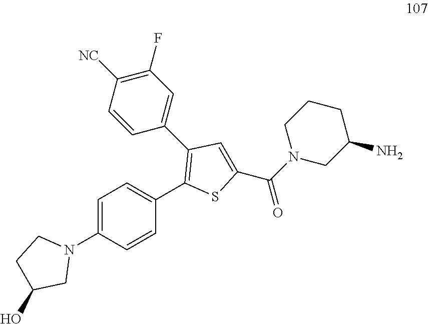 [SMILES: N#Cc1ccc(-c2cc(C(=O)N3CCC[C@@H](N)C3)sc2-c2ccc(N3CC[C@H](O)C3)cc2)cc1F]

Synthetic Route:

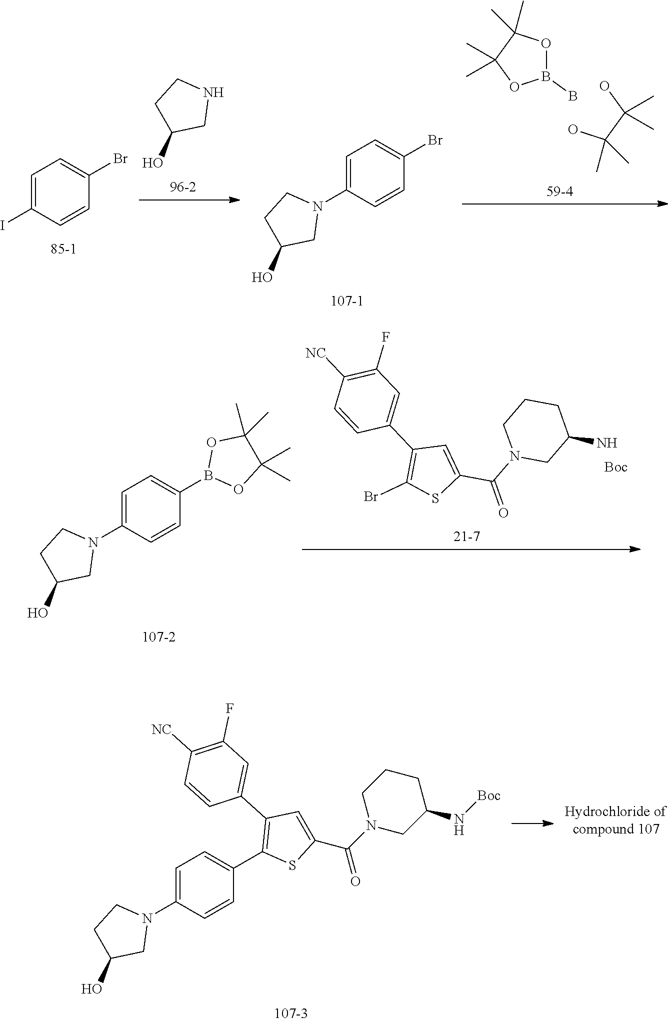

### Step 1

Under nitrogen atmosphere, compounds 85-1 (3.0 g, 10.6 mmol) and 96-2 (1.85 g, 21.2 mmol) were dissolved in N,N-dimethylethanolamine (30.00 mL), and then potassium phosphate monohydrate (4.88 g, 21.2 mmol) and cuprous iodide (404 mg, 2.12 mmol) were added thereto, and the reaction mixture was stirred and reacted for 12 hours at 55° C. The reaction mixture was added with saturated ammonium chloride (50 mL), and extracted with dichloromethane (50 mL×1). The organic phase was washed with saturated brine (50 mL×1), dried over anhydrous sodium sulfate, filtered, and concentrated under reduced pressure. The residue was purified by silica gel column chromatography (1/1, petroleum ether/ethyl acetate, R_{f}=0.26) to obtain compound 107-1. ¹H NMR (400 MHz, DMSO-d₆) δ 7.31-7.22 (m, 2H), 6.50-6.41 (m, 2H), 4.95 (d, J=3.6 Hz, 1H), 4.43-4.34 (m, 1H), 3.39-3.33 (m, 1H), 3.30-3.26 (m, 1H), 3.25-3.19 (m, 1H), 3.05-2.99 (m, 1H), 2.08-1.97 (m, 1H), 1.93-1.83 (m, 1H). MS-ESI calculated for [M+H]⁺ 242, 244, found 242, 244.

### Step 2

Under nitrogen atmosphere, compounds 107-1 (870 mg, 3.59 mmol), 59-4 (2.74 g, 10.8 mmol), and potassium acetate (1.06 g, 10.8 mmol) were dissolved in dioxane (10 mL), and the reaction mixture was added with [1,1′-bis(diphenylphosphino)ferrocene]dichloropalladium(II) (526 mg, 0.719 mmol), and the reaction mixture was stirred and reacted for 2 hours at 95° C., and concentrated under reduced pressure. The residue was purified by silica gel column chromatography (1/1, petroleum ether/ethyl acetate, R_{f}=0.25) to obtain compound 107-2. ¹H NMR (400 MHz, CDCl₃) δ 7.69 (d, J=8.4 Hz, 2H), 6.55 (d, J=8.4 Hz, 2H), 4.65-4.59 (m, 1H), 3.59-3.52 (m, 2H), 3.43-3.38 (m, 1H), 3.33-3.29 (m, 1H), 2.23-2.11 (m, 2H), 1.33 (s, 12H).

### Step 3

Under nitrogen atmosphere, compound 21-7 (700 mg, 1.38 mmol), compound 107-2 (439 mg, 1.52 mmol), and sodium carbonate (439 mg, 4.14 mmol) were dissolved in dioxane (8 mL) and water (2 mL), and the reaction mixture was added with [1,1′-bis(diphenylphosphino)ferrocene]dichloropalladium(II) (101 mg, 0.138 mmol). The reaction mixture was stirred and reacted for 12 hours at 100° C., added with water (20 mL), and extracted with ethyl acetate (20 mL×1). The organic phase was washed with saturated brine (20 mL×1), dried over anhydrous sodium sulfate, filtered, and concentrated under reduced pressure. The residue was purified by silica gel column chromatography (1/1, petroleum ether/ethyl acetate, R_{f}=0.21) to obtain compound 107-3. MS-ESI calculated for [M+H]⁺ 591, found 591.

### Step 4

Compound 107-3 (580 mg, 0.982 mmol) was dissolved in ethyl acetate (10 mL), and hydrochloride ethyl acetate solution (4 mol/L, 10 mL) was added dropwise thereto at 25° C. The reaction mixture was stirred for 1 hour at 25° C., concentrated under reduced pressure, and purified by preparative high performance liquid chromatography (chromatographic column: Phenomenex Genimi NX C18 150*40 mm*5 μm; mobile phase: mobile phase A: hydrochloric acid aqueous solution with a volume fraction of 0.05%; mobile phase B: acetonitrile; B %: 15 to 45%, 10 minutes) to obtain the hydrochloride of compound 107. ¹H NMR (400 MHz, CD₃OD) δ 7.68 (t, J=7.2 Hz, 1H), 7.55 (s, 1H), 7.37-7.22 (m, 4H), 6.92 (d, J=8.4 Hz, 2H), 4.65-4.55 (m, 1H), 4.43-4.36 (m, 1H), 4.21-4.11 (m, 1H), 3.68-3.61 (m, 2H), 3.56-3.37 (m, 5H), 2.32-2.17 (m, 2H), 2.16-2.06 (m, 1H), 1.96-1.86 (m, 1H), 1.84-1.66 (m, 2H). MS-ESI calculated for [M+H]⁺ 491, found 491.

### Example 108

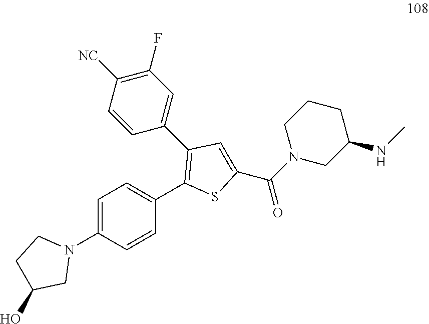 [SMILES: CN[C@@H]1CCCN(C(=O)c2cc(-c3ccc(C#N)c(F)c3)c(-c3ccc(N4CC[C@H](O)C4)cc3)s2)C1]

Synthetic Route:

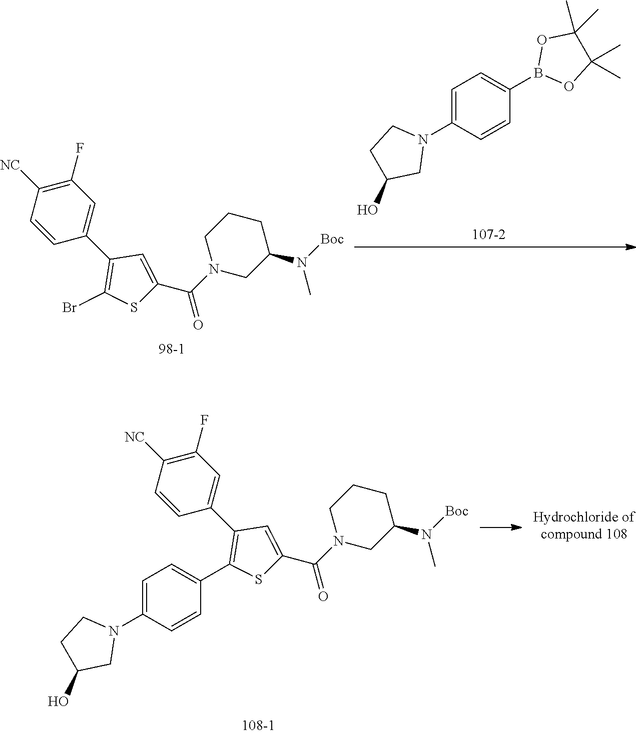 [SMILES: CN(C(=O)OC(C)(C)C)[C@@H]1CCCN(C(=O)c2cc(-c3ccc(C#N)c(F)c3)c(Br)s2)C1 ; CC1(C)OB(c2ccc(N3CC[C@H](O)C3)cc2)OC1(C)C ; CN(C(=O)OC(C)(C)C)[C@@H]1CCCN(C(=O)c2cc(-c3ccc(C#N)c(F)c3)c(-c3ccc(N4CC[C@H](O)C4)cc3)s2)C1]

### Step 1

Under nitrogen atmosphere, compound 98-1 (700 mg, 1.34 mmol), compound 107-2 (426 mg, 1.47 mmol), and sodium carbonate (426 mg, 4.02 mmol) were dissolved in dioxane (8 mL) and water (2 mL), and the reaction mixture was added with [1,1′-bis(diphenylphosphino)ferrocene]dichloropalladium(II) (98.0 mg, 0.134 mmol). The reaction mixture was stirred and reacted for 12 hours at 95° C., added with water (20 mL), and extracted with ethyl acetate (20 mL×1). The organic phase was washed with saturated brine (20 mL×1), dried over anhydrous sodium sulfate, filtered, and concentrated under reduced pressure. The residue was purified by silica gel column chromatography (1/1, petroleum ether/ethyl acetate, R_{f}=0.21) to obtain compound 108-1. MS-ESI calculated for [M+H]⁺ 605, found 605.

### Step 2

Compound 108-1 (400 mg, 0.661 mmol) was dissolved in ethyl acetate (10 mL), and hydrochloride ethyl acetate solution (4 mol/L, 10 mL) was added thereto at 25° C. The reaction mixture was stirred for 1 hour at 25° C., concentrated under reduced pressure, and purified by preparative high performance liquid chromatography (chromatographic column: Phenomenex Genimi NX C18 150*40 mm*5 μm; mobile phase: mobile phase A: hydrochloric acid aqueous solution with a volume fraction of 0.05%; mobile phase B: acetonitrile; B %: 12 to 42%, 10 minutes) to obtain the hydrochloride of compound 108. ¹H NMR (400 MHz, CD₃OD) δ 7.68 (t, J=7.2 Hz, 1H), 7.57 (s, 1H), 7.34-7.23 (m, 4H), 7.00 (d, J=8.4 Hz, 2H), 4.65-4.59 (m, 1H), 4.35-4.24 (m, 1H), 4.13-4.01 (m, 1H), 3.77-3.54 (m, 5H), 3.44-3.34 (m, 2H), 2.79 (s, 3H), 2.33-2.20 (m, 2H), 2.17-2.08 (m, 1H), 1.95-1.81 (m, 2H), 1.78-1.65 (m, 1H). MS-ESI calculated for [M+H]⁺ 505, found 505.

### Example 109

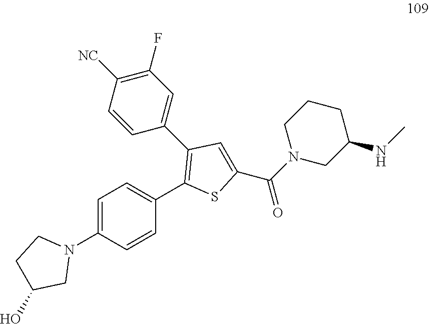 [SMILES: CN[C@@H]1CCCN(C(=O)c2cc(-c3ccc(C#N)c(F)c3)c(-c3ccc(N4CC[C@@H](O)C4)cc3)s2)C1]

Synthetic Route:

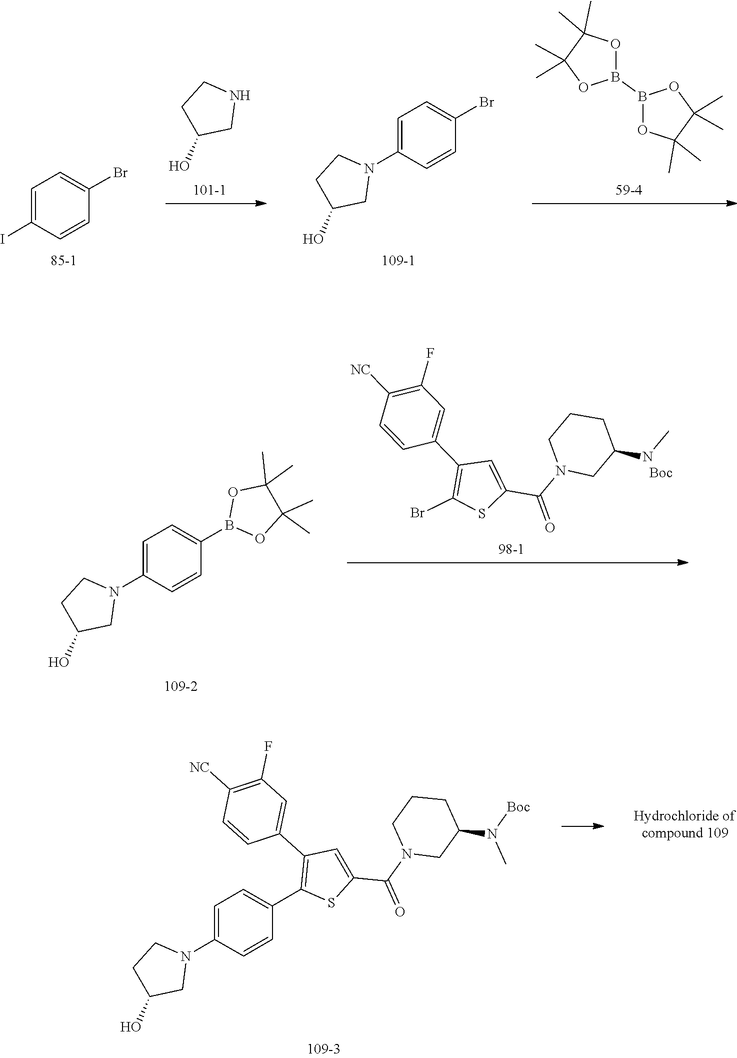

### Step 1

Under nitrogen atmosphere, compounds 85-1 (3.0 g, 10.6 mmol) and 101-1 (1.85 g, 21.2 mmol) were dissolved in N,N-dimethylethanolamine (30.0 mL), and then potassium phosphate monohydrate (4.88 g, 21.2 mmol) and cuprous iodide (404 mg, 2.12 mmol) were added thereto, and the reaction mixture was stirred and reacted for 12 hours at 55° C. The reaction mixture was added with saturated ammonium chloride (50 mL), and extracted with dichloromethane (50 mL×1). The organic phase was washed with saturated brine (50 mL×1), dried over anhydrous sodium sulfate, filtered, and concentrated under reduced pressure. The residue was purified by silica gel column chromatography (1/1, petroleum ether/ethyl acetate, R_{f}=0.24) to obtain compound 109-1. ¹H NMR (400 MHz, DMSO-d₆) δ 7.30-7.22 (m, 2H), 6.49-6.41 (m, 2H), 4.96 (d, J=3.6 Hz, 1H), 4.43-4.35 (m, 1H), 3.37 (d, J=4.8 Hz, 1H), 3.32-3.26 (m, 1H), 3.26-3.21 (m, 1H), 3.05-2.99 (m, 1H), 2.08-1.96 (m, 1H), 1.92-1.82 (m, 1H). MS-ESI calculated for [M+H]⁺ 242, 244, found 242, 244.

### Step 2

Under nitrogen atmosphere, compounds 109-1 (900 mg, 3.72 mmol), 59-4 (1.89 g, 7.43 mmol), and potassium acetate (1.09 g, 11.1 mmol) were dissolved in dioxane (10.0 mL), and the reaction mixture was added with [1,1′-bis(diphenylphosphino)ferrocene]dichloropalladium(II) (272 mg, 0.372 mmol), and the reaction mixture was stirred and reacted for 12 hours at 95° C., and concentrated under reduced pressure. The residue was purified by silica gel column chromatography (1/1, petroleum ether/ethyl acetate, R_{f}=0.25) to obtain compound 109-2. ¹H NMR (400 MHz, CDCl₃) δ 7.69 (d, J=8.4 Hz, 2H), 6.55 (d, J=8.8 Hz, 2H), 4.64-4.58 (m, 1H), 3.58-3.50 (m, 2H), 3.45-3.38 (m, 1H), 3.35-3.28 (m, 1H), 2.23-2.09 (m, 2H), 1.33 (s, 12H).

### Step 3

Under nitrogen atmosphere, compound 98-1 (550 mg, 1.05 mmol), compound 109-2 (335 mg, 1.16 mmol), and sodium carbonate (335 mg, 3.16 mmol) were dissolved in dioxane (8 mL) and water (2 mL), and the reaction mixture was added with [1,1′-bis(diphenylphosphino)ferrocene]dichloropalladium(II) (77.0 mg, 0.105 mmol). The reaction mixture was stirred and reacted for 12 hours at 95° C., added with water (20 mL), and extracted with ethyl acetate (20 mL×1). The organic phase was washed with saturated brine (20 mL×1), dried over anhydrous sodium sulfate, filtered, and concentrated under reduced pressure. The residue was purified by silica gel column chromatography (1/1, petroleum ether/ethyl acetate, R_{f}=0.21) to obtain compound 109-3. MS-ESI calculated for [M+H]⁺ 605, found 605.

### Step 4

Compound 109-3 (360 mg, 0.446 mmol) was dissolved in ethyl acetate (10 mL), and hydrochloride ethyl acetate solution (4 mol/L, 10 mL) was added thereto at 25° C. The reaction mixture was stirred for 1 hour at 25° C., concentrated under reduced pressure, and purified by preparative high performance liquid chromatography (chromatographic column: Phenomenex Genimi NX C18 150*40 mm*5 μm; mobile phase: mobile phase A: hydrochloric acid aqueous solution with a volume fraction of 0.05%; mobile phase B: acetonitrile; B %: 16 to 46%, 10 minutes) to obtain the hydrochloride of compound 109. ¹H NMR (400 MHz, CD₃OD) δ 7.67 (t, J=7.6 Hz, 1H), 7.58 (s, 1H), 7.35-7.26 (m, 4H), 7.13 (d, J=9.2 Hz, 2H), 4.67-4.61 (m, 1H), 4.37-4.26 (m, 1H), 4.14-4.01 (m, 1H), 3.77-3.57 (m, 5H), 3.49-3.43 (m, 1H), 3.42-3.34 (m, 1H), 2.79 (s, 3H), 2.36-2.21 (m, 2H), 2.20-2.10 (m, 1H), 1.97-1.82 (m, 2H), 1.79-1.64 (m, 1H). MS-ESI calculated for [M+H]⁺ 505, found 505.

### Example 110

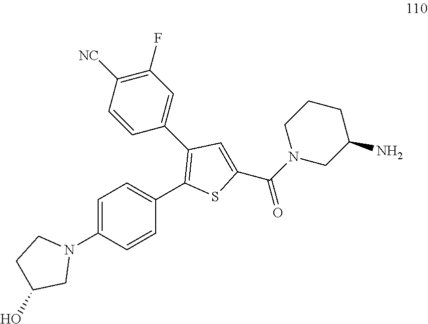 [SMILES: N#Cc1ccc(-c2cc(C(=O)N3CCC[C@@H](N)C3)sc2-c2ccc(N3CC[C@@H](O)C3)cc2)cc1F]

Synthetic Route:

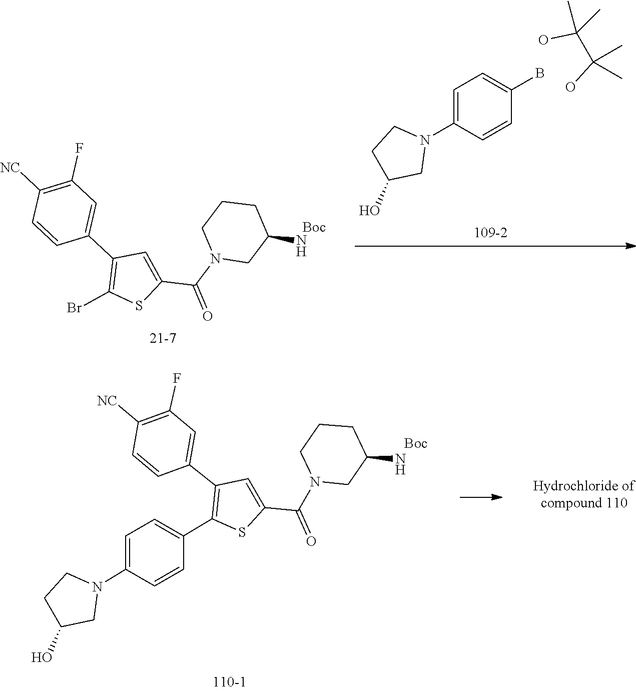

### Step 1

Under nitrogen atmosphere, compound 21-7 (600 mg, 1.18 mmol), compound 109-2 (375 mg, 1.30 mmol), and sodium carbonate (375 mg, 3.54 mmol) were dissolved in dioxane (8 mL) and water (2 mL), and the reaction mixture was added with [1,1′-bis(di-tert-butylphosphino)ferrocene]dichloropalladium(II) (76.9 mg, 0.118 mmol). The reaction mixture was stirred and reacted for 12 hours at 95° C., added with water (20 mL), and extracted with ethyl acetate (20 mL×1). The organic phase was washed with saturated brine (20 mL×1), dried over anhydrous sodium sulfate, filtered, and concentrated under reduced pressure. The residue was purified by silica gel column chromatography (1/1, petroleum ether/ethyl acetate, R_{f}=0.21) to obtain compound 110-1. MS-ESI calculated for [M+H]⁺ 591, found 591.

### Step 2

Compound 110-1 (300 mg, 0.508 mmol) was dissolved in ethyl acetate (10 mL), and hydrochloride ethyl acetate solution (4 mol/L, 10 mL) was added dropwise thereto at 25° C. The reaction mixture was stirred for 1 hour at 25° C., concentrated under reduced pressure, and separated by preparative high performance liquid chromatography (chromatographic column: Phenomenex Genimi NX C18 150*40 mm*5 μm; mobile phase: mobile phase A: hydrochloric acid aqueous solution with a volume fraction of 0.05%; mobile phase B: acetonitrile; B %: 10 to 40%, 10 minutes) to obtain the hydrochloride of compound 110. ¹H NMR (400 MHz, CD₃OD) δ 7.68 (t, J=7.6 Hz, 1H), 7.54 (s, 1H), 7.34-7.27 (m, 2H), 7.21 (J=8.4 Hz, 2H), 6.81 (d, J=8.4 Hz, 2H), 4.63-4.53 (m, 1H), 4.46-4.34 (m, 1H), 4.22-4.10 (m, 1H), 3.64-3.55 (m, 2H), 3.54-3.33 (m, 5H), 2.28-2.16 (m, 2H), 2.15-2.05 (m, 1H), 1.97-1.85 (m, 1H), 1.83-1.66 (m, 2H). MS-ESI calculated for [M+H]⁺ 491, found 491.

### Example 111

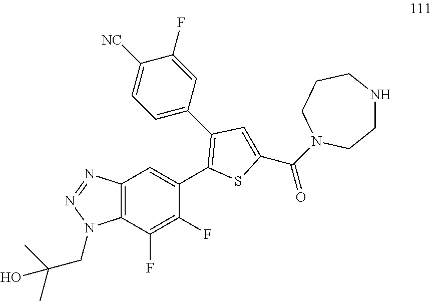 [SMILES: CC(C)(O)Cn1nnc2cc(-c3sc(C(=O)N4CCCNCC4)cc3-c3ccc(C#N)c(F)c3)c(F)c(F)c21]

Synthetic Route:

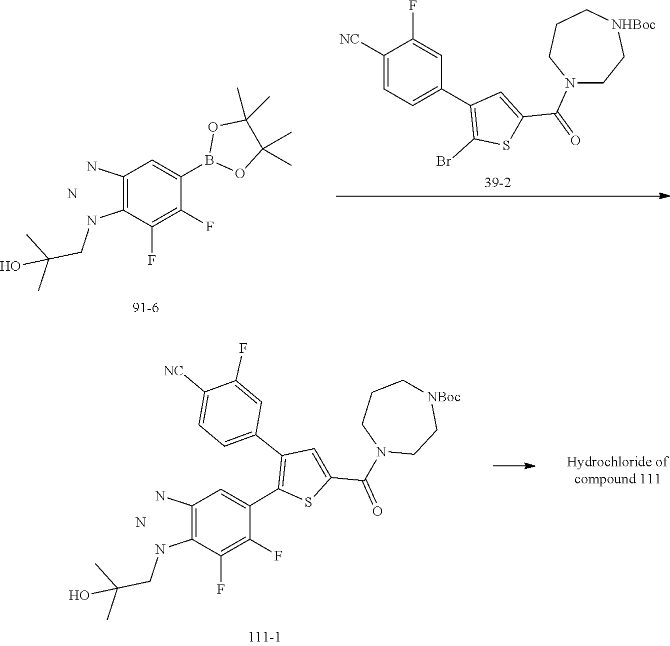

### Step 1

Under nitrogen atmosphere, compound 91-6 (625 mg, 1.77 mmol), compound 39-2 (600 mg, 1.18 mmol), and potassium phosphate (501 mg, 2.36 mmol) were dissolved in dioxane (10.0 mL) and water (2.5 mL), and the reaction mixture was added with [1,1′-bis(di-tert-butylphosphino)ferrocene]dichloropalladium(II) (77.0 mg, 118 μmol), stirred, and reacted for 2 hours at 90° C. The reaction mixture was concentrated under reduced pressure, and the residue was purified by silica gel column chromatography (1/1, petroleum ether/ethyl acetate, R_{f}=0.33) to obtain compound 111-1. MS-ESI calculated for [M−56+H]⁺ 599, found 599.

### Step 2

Compound 111-1 (565 mg, 863 μmol) was dissolved in ethyl acetate (2.00 mL), and hydrochloride ethyl acetate solution (4 mol/L, 10.0 mL) was added thereto, and the reaction mixture was stirred and reacted for 0.5 hours at 25° C. After the reaction mixture was concentrated, the residue was purified by preparative high performance liquid chromatography (chromatographic column: Xtimate C18 150*40 mm*5 μm, mobile phase A: hydrochloric acid aqueous solution with a volume fraction of 0.05%; mobile phase B: acetonitrile; B %: 10% to 45%, 10 minutes) to obtain the hydrochloride of compound 111. ¹H NMR (400 MHz, CD₃OD) δ=(d, J=5.2 Hz, 1H), 7.76 (s, 1H), 7.70-7.63 (m, 1H), 7.40 (d, J=10.4 Hz, 1H), 7.25 (d, J=8.4 Hz, 1H), 4.77 (s, 2H), 4.20-3.93 (m, 4H), 3.56-3.46 (m, 2H), 3.45-3.39 (m, 2H), 2.34-2.21 (m, 2H), 1.31 (s, 6H). MS-ESI calculated for [M+H]⁺ 555, found 555.

### Example 112

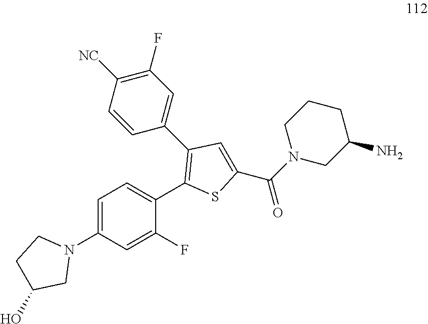 [SMILES: N#Cc1ccc(-c2cc(C(=O)N3CCC[C@@H](N)C3)sc2-c2ccc(N3CC[C@@H](O)C3)cc2F)cc1F]

Synthetic Route:

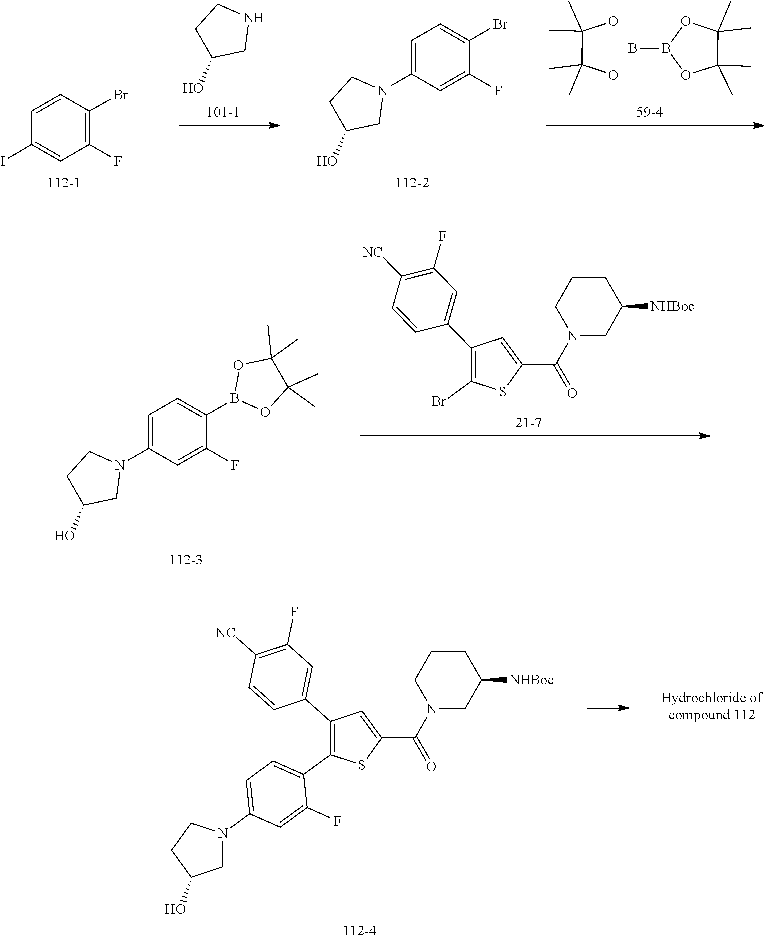

### Step 1

Compounds 112-1 (5.00 g, 16.6 mmol) and 101-1 (2.90 g, 33.23 mmol) were dissolved in N,N-dimethylethanolamine (17.0 mL), and then potassium phosphate (7.05 g, 33.23 mmol) and cuprous iodide (316 mg, 1.66 mmol) were added thereto. Under nitrogen atmosphere, the reaction mixture was stirred and reacted for 12 hours at 55° C. The reaction mixture was added with water (150 mL), and extracted with dichloromethane (150 mL×2). The organic phase was washed with saturated brine (100 mL×2), dried over anhydrous sodium sulfate, filtered, and concentrated under reduced pressure. The residue was purified by silica gel column chromatography (1/1, petroleum ether/ethyl acetate, R_{f}=0.35) to obtain compound 112-2. MS-ESI calculated for [M+H]⁺ 260, found 260.

### Step 2

Under nitrogen atmosphere, compounds 112-2 (1.80 g, 6.92 mmol), 59-4 (5.27 g, 20.76 mmol), and potassium acetate (2.04 g, 20.76 mmol) were dissolved in dioxane (60.0 mL), and the reaction mixture was added with [1,1′-bis(diphenylphosphino)ferrocene]dichloropalladium(II) (1.01 g, 1.38 mol). The reaction mixture was stirred and reacted for 12 hours at 90° C. The reaction mixture was concentrated under reduced pressure, and the residue was purified by silica gel column chromatography (1/1, petroleum ether/ethyl acetate, R_{f}=0.48) to obtain compound 112-3. MS-ESI calculated for [M+H]⁺ 308, found 308.

### Step 3

Under nitrogen atmosphere, compound 21-7 (413 mg, 813 μmol), compound 112-3 (250 mg, 813 μmol), and sodium carbonate (172 mg, 1.63 mmol) were dissolved in dioxane (6.00 mL) and water (1.50 mL), and the reaction mixture was added with [1,1′-bis(diphenylphosphino)ferrocene]dichloropalladium(II) (59.0 mg, 81 μmol), and the reaction mixture was stirred and reacted for 12 hours at 90° C. The reaction mixture was concentrated under reduced pressure, and the residue was purified by silica gel column chromatography (1/1, petroleum ether/ethyl acetate, R_{f}=0.60) to obtain compound 112-4. MS-ESI calculated for [M+H]⁺ 609, found 609.

### Step 4

Compound 112-4 (410 mg, 673 μmol) was dissolved in ethyl acetate (2.0 mL), and hydrochloride ethyl acetate solution (4 mol/L, 10.0 mL) was added thereto, and the reaction mixture was stirred and reacted for 20 minutes at 25° C. After the reaction mixture was concentrated, the residue was purified by preparative high performance liquid chromatography (chromatographic column: Xtimate C18 150*40 mm*5 μm, mobile phase A: hydrochloric acid aqueous solution with a volume fraction of 0.05%; mobile phase B: acetonitrile; B %: 15% to 45%, 10 minutes) to obtain the hydrochloride of compound 112. ¹H NMR (400 MHz, CD₃OD) δ=7.68 (d, J=8.4 Hz, 1H), 7.61 (s, 1H), 7.33-7.26 (m, 2H), 7.17 (t, J=8.4 Hz, 1H), 6.45 (d, J=8.4 Hz, 1H), 6.30 (d, J=13.2 Hz, 1H), 4.59-4.52 (m, 2H), 4.42-4.34 (m, 1H), 4.20-4.12 (m, 1H), 3.55-3.48 (m, 3H), 3.46-3.39 (m, 2H), 3.27-3.22 (m, 1H), 2.26-2.13 (m, 2H), 2.11-2.02 (m, 1H), 1.93 (m, 1H), 1.76 (m, 2H). MS-ESI calculated for [M+H]⁺ 509, found 509.

### Example 113

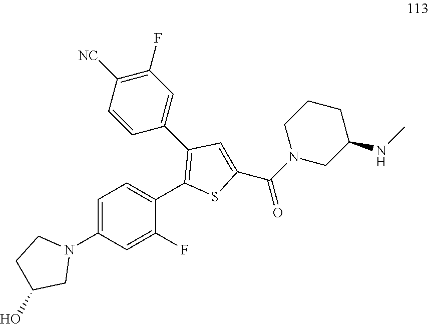 [SMILES: CN[C@@H]1CCCN(C(=O)c2cc(-c3ccc(C#N)c(F)c3)c(-c3ccc(N4CC[C@@H](O)C4)cc3F)s2)C1]

Synthetic Route:

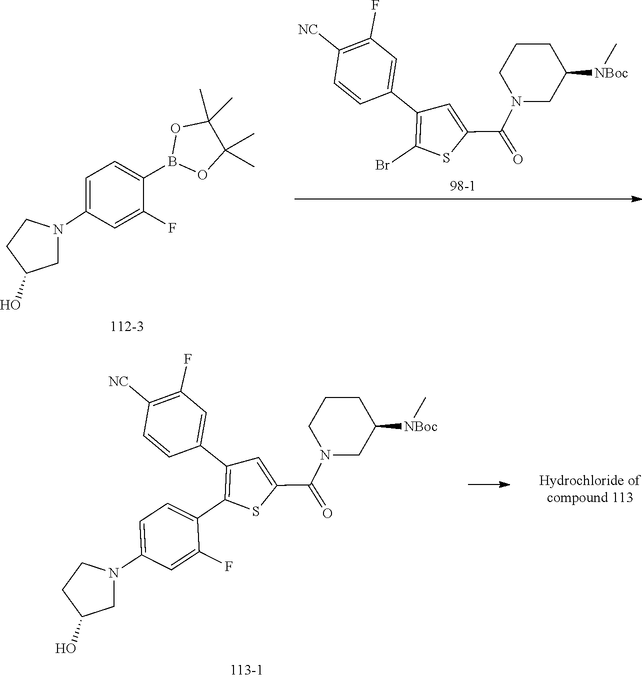 [SMILES: CC1(C)OB(c2ccc(N3CC[C@@H](O)C3)cc2F)OC1(C)C ; CN(C(=O)OC(C)(C)C)[C@@H]1CCCN(C(=O)c2cc(-c3ccc(C#N)c(F)c3)c(Br)s2)C1 ; CN(C(=O)OC(C)(C)C)[C@@H]1CCCN(C(=O)c2cc(-c3ccc(C#N)c(F)c3)c(-c3ccc(N4CC[C@@H](O)C4)cc3F)s2)C1]

### Step 1

Under nitrogen atmosphere, compound 98-1 (425 mg, 813 μmol), compound 112-3 (250 mg, 813 μmol), and sodium carbonate (172 mg, 1.63 mmol) were dissolved in dioxane (6.00 mL) and water (1.50 mL), and the reaction mixture was added with [1,1′-bis(diphenylphosphino)ferrocene]dichloropalladium(II) (59.0 mg, 81.0 μmol), and the reaction mixture was stirred and reacted for 12 hours at 90° C. The reaction mixture was concentrated under reduced pressure, and the residue was purified by silica gel column chromatography (1/1, petroleum ether/ethyl acetate, R_{f}=0.52) to obtain compound 113-1. MS-ESI calculated for [M+H]⁺ 623, found 623.

### Step 2

Compound 113-1 (400 mg, 642 μmol) was dissolved in ethyl acetate (2.00 mL), and hydrochloride ethyl acetate solution (4 mol/L, 9.80 mL) was added thereto, and the reaction mixture was stirred and reacted for 20 minutes at 25° C. After the reaction mixture was concentrated, the residue was purified by preparative high performance liquid chromatography (chromatographic column: Phenomenex C18 150*40 mm*5 Vim, mobile phase A: hydrochloric acid aqueous solution with a volume fraction of 0.05%; mobile phase B: acetonitrile; B %: 12% to 42%, 10 minutes) to obtain the hydrochloride of compound 113. ¹H NMR (400 MHz, CD₃OD) δ=7.70-7.65 (m, 1H), 7.62 (s, 1H), 7.32-7.25 (m, 2H), 7.16 (t, J=8.8 Hz, 1H), 6.45 (d, J=8.4 Hz, 1H), 6.31 (d, J=13.6 Hz, 1H), 4.59-4.50 (m, 1H), 4.32-4.20 (m, 1H), 4.14-4.04 (m, 1H), 3.80-3.67 (m, 2H), 3.55-3.45 (m, 2H), 3.43-3.36 (m, 2H), 3.28-3.21 (m, 1H), 2.81 (s, 3H), 2.30-2.14 (m, 2H), 2.11-2.02 (m, 1H), 1.96-1.81 (m, 2H), 1.80-1.69 (m, 1H). MS-ESI calculated for [M+H]⁺ 523, found 523.

### Example 114

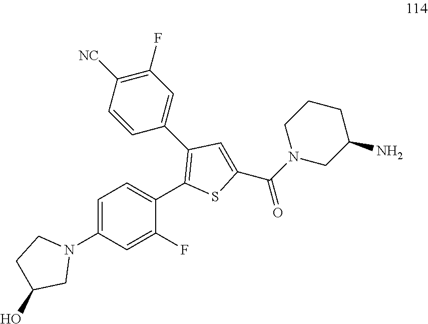 [SMILES: N#Cc1ccc(-c2cc(C(=O)N3CCC[C@@H](N)C3)sc2-c2ccc(N3CC[C@H](O)C3)cc2F)cc1F]

Synthetic Route:

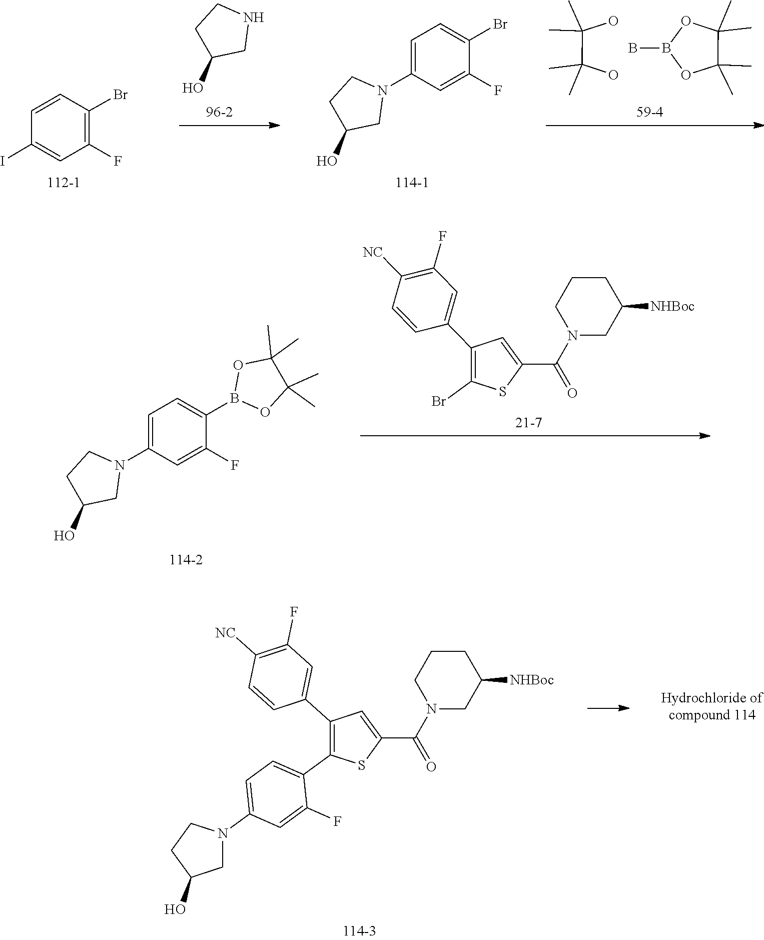

### Step 1

Compounds 112-1 (4.70 g, 15.6 mmol) and 96-2 (2.72 g, 31.24 mmol) were dissolved in N,N-dimethylethanolamine (16.0 mL), and then potassium phosphate (6.63 g, 31.24 mmol) and cuprous iodide (297 mg, 1.56 mmol) were added thereto, and the reaction mixture was stirred and reacted for 12 hours at 55° C., added with water (200 mL), extracted with dichloromethane (100 mL×2). The organic phase was washed with saturated brine (100 mL×1), dried over anhydrous sodium sulfate, filtered, and concentrated under reduced pressure. The residue was purified by silica gel column chromatography (1/1, petroleum ether/ethyl acetate, R_{f}=0.32) to obtain compound 114-1. ¹H NMR (400 MHz, CD₃OD) δ=7.29 (t, J=8.4 Hz, 1H), 6.41-6.26 (m, 2H), 4.57-4.49 (m, 1H), 3.49-3.38 (m, 2H), 3.32-3.28 (m, 1H), 3.21-3.15 (m, 1H), 2.23-2.10 (m, 1H), 2.09-1.98 (m, 1H). MS-ESI calculated for [M+H]⁺ 260, found 260.

### Step 2

Under nitrogen atmosphere, compounds 114-1 (2.56 g, 9.84 mmol), 59-4 (7.50 g, 29.53 mmol), and potassium acetate (2.90 g, 29.5 mmol) were dissolved in dioxane (60.0 mL), and the reaction mixture was added with [1,1′-bis(diphenylphosphino)ferrocene]dichloropalladium(II) (720 mg, 984 μmol). The reaction mixture was stirred and reacted for 12 hours at 90° C. The reaction mixture was concentrated under reduced pressure, and the residue was purified by silica gel column chromatography (1/1, petroleum ether/ethyl acetate, R_{f}=0.68) to obtain compound 114-2. MS-ESI calculated for [M+H]⁺ 308, found 308.

### Step 3

Under nitrogen atmosphere, compound 21-7 (496 mg, 976 μmol), compound 114-2 (300 mg, 976 μmol), and sodium carbonate (207 mg, 1.95 mmol) were dissolved in dioxane (6.00 mL) and water (1.50 mL), and the reaction mixture was added with [1,1′-bis(diphenylphosphino)ferrocene]dichloropalladium(II) (71.0 mg, 97.0 μmol), and the reaction mixture was stirred and reacted for 12 hours at 90° C. The reaction mixture was concentrated under reduced pressure, and the residue was purified by silica gel column chromatography (1/1, petroleum ether/ethyl acetate, R_{f}=0.51) to obtain compound 114-3. MS-ESI calculated for [M+H]⁺ 609, found 609.

### Step 4

Compound 114-3 (450 mg, 739 μmol) was dissolved in ethyl acetate (2.00 mL), and hydrochloride ethyl acetate solution (4 mol/L, 11.2 mL) was added thereto, and the reaction mixture was stirred and reacted for 20 minutes at 25° C. After the reaction mixture was concentrated, the residue was purified by preparative high performance liquid chromatography (chromatographic column: Xtimate C18 150*40 mm*5 μm, mobile phase A: hydrochloric acid aqueous solution with a volume fraction of 0.05%; mobile phase B: acetonitrile; B %: 15% to 45%, 10 minutes) to obtain the hydrochloride of compound 114. ¹H NMR (400 MHz, CD₃OD) δ=7.70-7.65 (m, 1H), 7.61 (s, 1H), 7.33-7.26 (m, 2H), 7.17 (t, J=8.4 Hz, 1H), 6.45 (dd, J=2.0, 8.8 Hz, 1H), 6.31 (dd, J=2.4, 13.6 Hz, 1H), 4.59-4.53 (m, 1H), 4.43-4.35 (m, 1H), 4.21-4.12 (m, 1H), 3.58-3.37 (m, 6H), 3.28-3.22 (m, 1H), 2.27-2.13 (m, 2H), 2.11-2.02 (m, 1H), 1.97-1.87 (m, 1H), 1.85-1.69 (m, 2H). MS-ESI calculated for [M+H]⁺ 509, found 509.

### Example 115

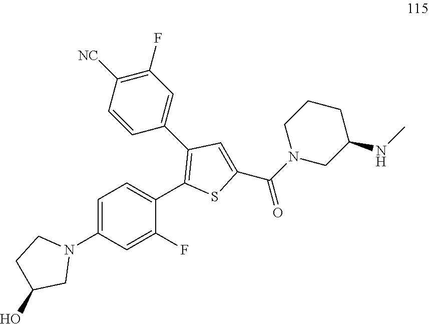 [SMILES: CN[C@@H]1CCCN(C(=O)c2cc(-c3ccc(C#N)c(F)c3)c(-c3ccc(N4CC[C@H](O)C4)cc3F)s2)C1]

Synthetic Route:

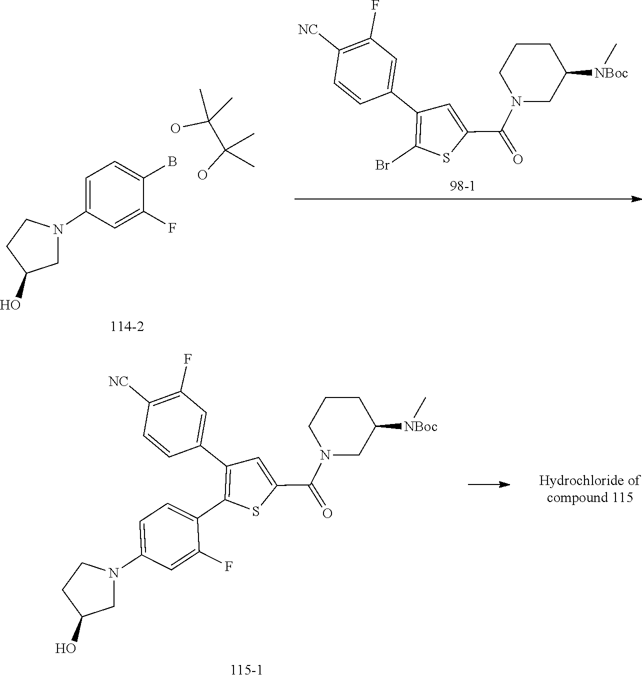

### Step 1

Under nitrogen atmosphere, compound 98-1 (679 mg, 1.30 mmol), compound 114-2 (400 mg, 1.30 mmol), and sodium carbonate (275 mg, 2.60 mmol) were dissolved in dioxane (6.00 mL) and water (1.50 mL), and the reaction mixture was added with [1,1′-bis(diphenylphosphino)ferrocene]dichloropalladium(II) (95.0 mg, 130 μmol), and the reaction mixture was stirred and reacted for 12 hours at 90° C. The reaction mixture was concentrated under reduced pressure, and the residue was purified by silica gel column chromatography (1/1, petroleum ether/ethyl acetate, R_{f}=0.52) to obtain compound 115-1. MS-ESI calculated for [M+H]⁺ 623, found 623.

### Step 2

Compound 115-1 (550 mg, 883 μmol) was dissolved in ethyl acetate (2.00 mL), and hydrochloride ethyl acetate solution (4 mol/L, 10.0 mL) was added thereto, and the reaction mixture was stirred and reacted for 20 minutes at 25° C. After the reaction mixture was concentrated, the residue was purified by preparative high performance liquid chromatography (chromatographic column: Xtimate C18 150*40 mm*5 μm, mobile phase A: hydrochloric acid aqueous solution with a volume fraction of 0.05%; mobile phase B: acetonitrile; B %: 14% to 44%, 10 minutes) to obtain the hydrochloride of compound 115. ¹H NMR (400 MHz, CD₃OD) δ=7.70-7.65 (m, 1H), 7.63 (s, 1H), 7.32-7.29 (m, 1H), 7.28 (s, 1H), 7.17 (t, J=8.4 Hz, 1H), 6.46 (dd, J=2.4, 8.4 Hz, 1H), 6.32 (dd, J=2.0, 13.6 Hz, 1H), 4.61-4.50 (m, 1H), 4.35-4.21 (m, 1H), 4.15-4.03 (m, 1H), 3.80-3.62 (m, 2H), 3.57-3.46 (m, 2H), 3.44-3.36 (m, 2H), 3.29-3.21 (m, 1H), 2.81 (s, 3H), 2.32-2.02 (m, 3H), 1.97-1.82 (m, 2H), 1.81-1.67 (m, 1H). MS-ESI calculated for [M+H]⁺ 523, found 523.

### Example 116

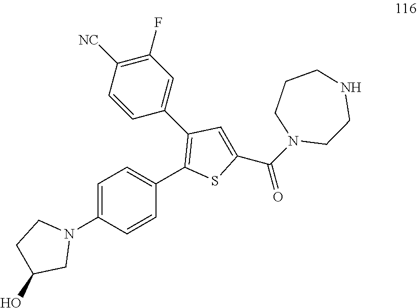 [SMILES: N#Cc1ccc(-c2cc(C(=O)N3CCCNCC3)sc2-c2ccc(N3CC[C@H](O)C3)cc2)cc1F]

Synthetic Route:

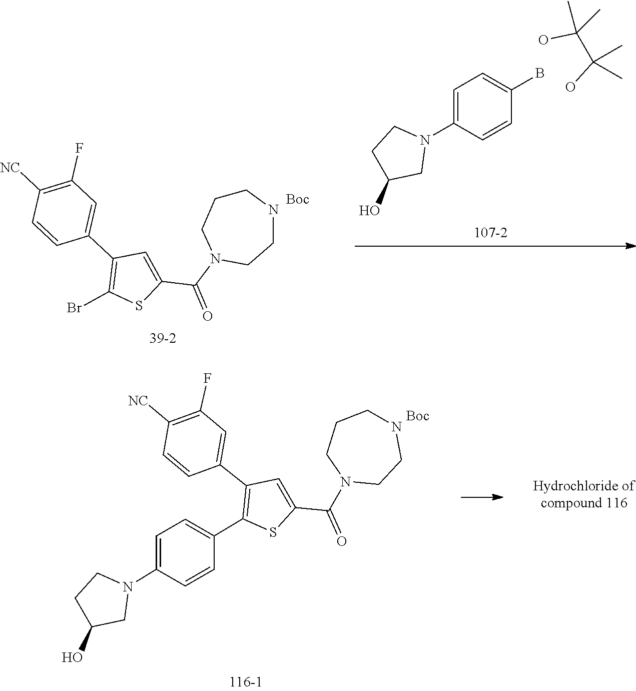

### Step 1

Under nitrogen atmosphere, compound 39-2 (600 mg, 1.18 mmol), compound 107-2 (375 mg, 1.30 mmol), and sodium carbonate (375 mg, 3.54 mmol) were dissolved in dioxane (8.00 mL) and water (2.00 mL), and the reaction mixture was added with [1,1′-bis(di-tert-butylphosphino)ferrocene]dichloropalladium(II) (76.9 mg, 0.118 mmol). The reaction mixture was stirred and reacted for 12 hours at 95° C., added with water (20 mL), and extracted with ethyl acetate (20 mL×1). The organic phase was washed with saturated brine (20 mL×1), dried over anhydrous sodium sulfate, filtered, and concentrated under reduced pressure. The residue was purified by silica gel column chromatography (1/1, petroleum ether/ethyl acetate, R_{f}=0.21) to obtain compound 116-1. MS-ESI calculated for [M+H]⁺ 591, found 591.

### Step 2

Compound 116-1 (700 mg, 0.782 mmol) was dissolved in ethyl acetate (10 mL), and hydrochloride ethyl acetate solution (4 mol/L, 10 mL) was added dropwise thereto at 25° C. The reaction mixture was stirred for 1 hour at 25° C., concentrated under reduced pressure, and separated by preparative high performance liquid chromatography (chromatographic column: Phenomenex Genimi NX C18 150*40 mm*5 μm; mobile phase: mobile phase A: hydrochloric acid aqueous solution with a volume fraction of 0.05%; mobile phase B: acetonitrile; B %: 10 to 40%, 10 minutes) to obtain the hydrochloride of compound 116. ¹H NMR (400 MHz, CD₃OD) δ 7.71-7.64 (m, 1H), 7.59 (s, 1H), 7.33-7.26 (m, 2H), 7.22 (d, J=8.4 Hz, 2H), 6.86 (d, J=8.4 Hz, 2H), 4.62-4.56 (m, 1H), 4.15-3.91 (m, 4H), 3.66-3.57 (m, 2H), 3.55-3.44 (m, 3H), 3.41-3.34 (m, 3H), 2.30-2.19 (m, 3H), 2.15-2.05 (m, 1H). MS-ESI calculated for [M+H]⁺ 491, found 491.

### Example 117

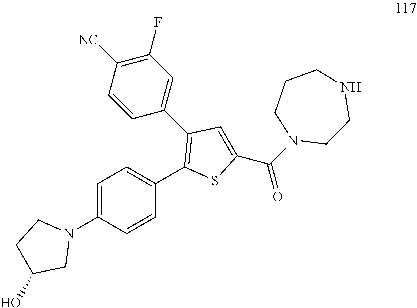 [SMILES: N#Cc1ccc(-c2cc(C(=O)N3CCCNCC3)sc2-c2ccc(N3CC[C@@H](O)C3)cc2)cc1F]

Synthetic Route:

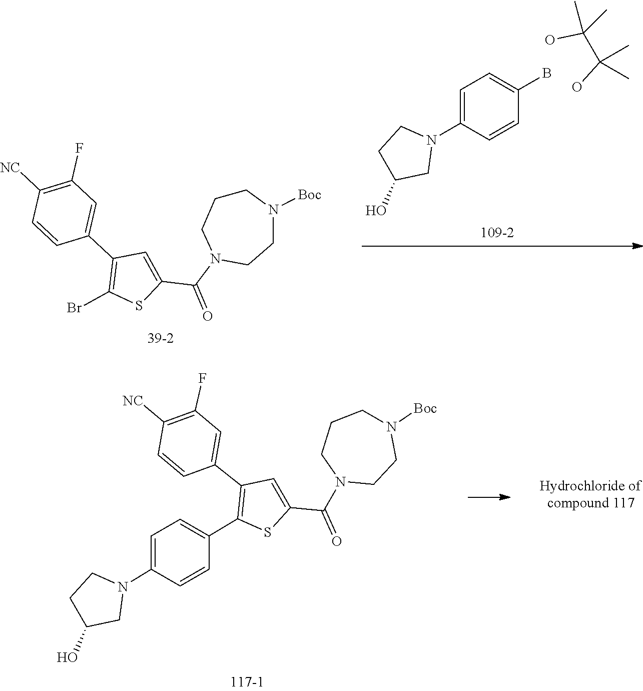

### Step 1

Under nitrogen atmosphere, compound 39-2 (700 mg, 1.38 mmol), compound 109-2 (438 mg, 1.51 mmol), and sodium carbonate (438 mg, 4.13 mmol) were dissolved in dioxane (8.00 mL) and water (2.00 mL), and the reaction mixture was added with [1,1′-bis(di-tert-butylphosphino)ferrocene]dichloropalladium(II) (90.0 mg, 0.118 mmol). The reaction mixture was stirred and reacted for 12 hours at 95° C., added with water (20 mL), and extracted with ethyl acetate (20 mL×1). The organic phase was washed with saturated brine (20 mL×1), dried over anhydrous sodium sulfate, filtered, and concentrated under reduced pressure. The residue was purified by silica gel column chromatography (1/1, petroleum ether/ethyl acetate, R_{f}=0.21) to obtain compound 117-1. MS-ESI calculated for [M+H]⁺ 591, found 591.

### Step 2

Compound 117-1 (600 mg, 0.741 mmol) was dissolved in ethyl acetate (10.0 mL), and hydrochloride ethyl acetate solution (4 mol/L, 10.0 mL) was added dropwise thereto at 25° C. The reaction mixture was stirred for 1 hour at 25° C., concentrated under reduced pressure, and purified by preparative high performance liquid chromatography (chromatographic column: Phenomenex Genimi NX C18 150*40 mm*5 μm; mobile phase: mobile phase A: hydrochloric acid aqueous solution with a volume fraction of 0.05%; mobile phase B: acetonitrile; B %: 12 to 42%, 10 minutes) to obtain the hydrochloride of compound 117. ¹H NMR (400 MHz, CD₃OD) δ 7.68 (t, J=7.2 Hz, 1H), 7.60 (s, 1H), 7.33-7.24 (m, 4H), 6.97 (d, J=8.4 Hz, 2H), 4.65-4.58 (m, 1H), 4.14-3.88 (m, 4H), 3.70-3.61 (m, 2H), 3.61-3.53 (m, 1H), 3.52-3.43 (m, 2H), 3.42-3.35 (m, 3H), 2.34-2.18 (m, 3H), 2.17-2.08 (m, 1H). MS-ESI calculated for [M+H]⁺ 491, found 491.

### Example 118

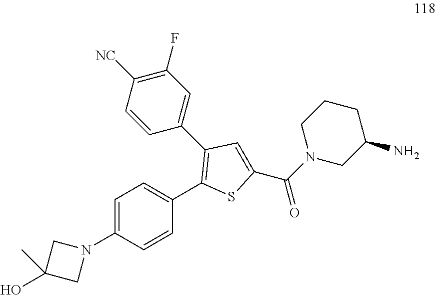 [SMILES: CC1(O)CN(c2ccc(-c3sc(C(=O)N4CCC[C@@H](N)C4)cc3-c3ccc(C#N)c(F)c3)cc2)C1]

Synthetic Route:

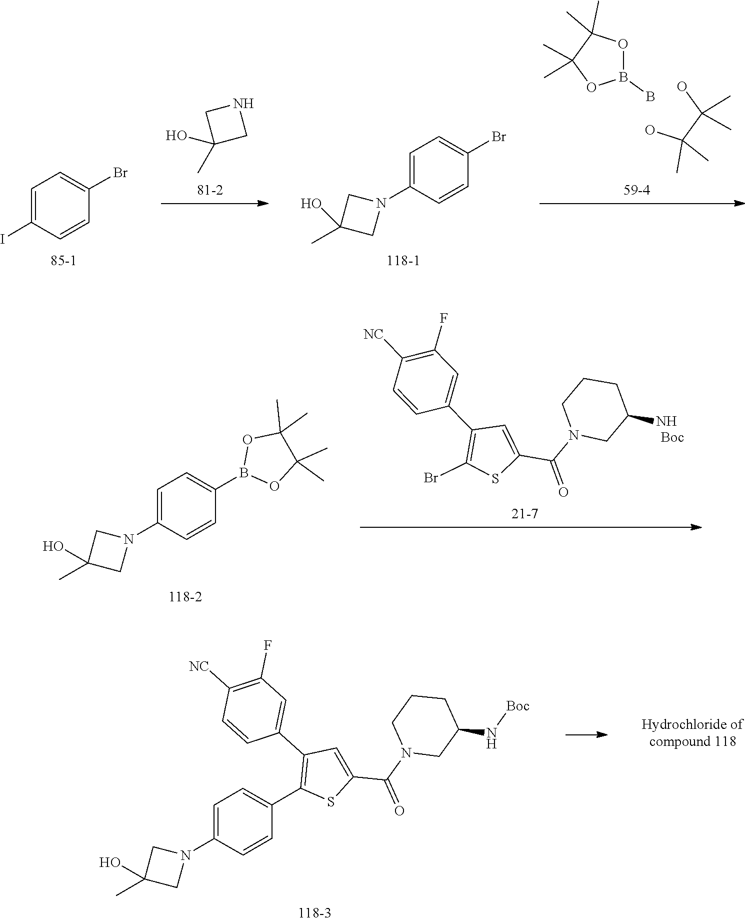

### Step 1

Under nitrogen atmosphere, compounds 85-1 (2.5 g, 8.84 mmol) and 81-2 (2.18 g, 17.7 mmol) were dissolved in N,N-dimethylethanolamine (20.0 mL), and then potassium phosphate monohydrate (8.14 g, 35.4 mmol) and cuprous iodide (337 mg, 1.77 mmol) were added thereto, and the reaction mixture was stirred and reacted for 12 hours at 55° C. The reaction mixture was added with saturated ammonium chloride (50 mL), and extracted with dichloromethane (50 mL×1). The organic phase was washed with saturated brine (50 mL×1), dried over anhydrous sodium sulfate, filtered, and concentrated under reduced pressure. The residue was purified by silica gel column chromatography (1/1, petroleum ether/ethyl acetate, R_{f}=0.24) to obtain compound 118-1. ¹H NMR (400 MHz, DMSO-d₆) δ 7.30-7.25 (m, 2H), 6.40-6.34 (m, 2H), 5.53 (s, 1H), 3.72 (d, J=7.6 Hz, 2H), 3.57 (d, J=7.6 Hz, 2H), 1.43 (s, 3H).

### Step 2 ES18490-238

Under nitrogen atmosphere, compounds 118-1 (1.43 g, 5.91 mmol), 59-4 (3.00 g, 11.8 mmol), and potassium acetate (1.74 g, 17.7 mmol) were dissolved in dioxane (10.0 mL), and the reaction mixture was added with [1,1′-bis(diphenylphosphino)ferrocene]dichloropalladium(II) (432 mg, 0.591 mmol), and the reaction mixture was stirred and reacted for 12 hours at 95° C., and concentrated under reduced pressure. The residue was purified by silica gel column chromatography (1/1, petroleum ether/ethyl acetate, R_{f}=0.25) to obtain compound 118-2. ¹H NMR (400 MHz, CDCl₃) δ 7.68 (d, J=8.4 Hz, 2H), 6.44 (d, J=8.4 Hz, 2H), 3.91-3.86 (m, 2H), 3.82-3.77 (m, 2H), 1.60 (s, 3H), 1.33 (s, 12H). MS-ESI calculated for [M+H]⁺ 290, found 290.

### Step 3

Under nitrogen atmosphere, compound 21-7 (800 mg, 1.57 mmol), compound 118-2 (501 mg, 1.73 mmol), and sodium carbonate (500 mg, 4.72 mmol) were dissolved in dioxane (8.00 mL) and water (2.00 mL), and the reaction mixture was added with [1,1′-bis(di-tert-butylphosphino)ferrocene]dichloropalladium(II) (103 mg, 0.157 mmol). The reaction mixture was stirred and reacted for 12 hours at 100° C., added with water (20 mL), and extracted with ethyl acetate (20 mL×1). The organic phase was washed with saturated brine (20 mL×1), dried over anhydrous sodium sulfate, filtered, and concentrated under reduced pressure. The residue was purified by silica gel column chromatography (1/1, petroleum ether/ethyl acetate, R_{f}=0.21) to obtain compound 118-3. MS-ESI calculated for [M+H]⁺ 591, found 591.

### Step 4

Compound 118-3 (780 mg, 1.32 mmol) was dissolved in ethyl acetate (10.0 mL), and hydrochloride ethyl acetate solution (4 mol/L, 10.0 mL) was added dropwise thereto at 25° C. The reaction mixture was stirred for 1 hour at 25° C., concentrated under reduced pressure, and purified by preparative high performance liquid chromatography (chromatographic column: Phenomenex Genimi NX C18 150*40 mm*5 μm; mobile phase: mobile phase A: hydrochloric acid aqueous solution with a volume fraction of 0.05%; mobile phase B: acetonitrile; B %: 12 to 42%, 10 minutes) to obtain the hydrochloride of compound 118. ¹H NMR (400 MHz, CD₃OD) δ 7.72-7.66 (m, 1H), 7.57 (s, 1H), 7.35-7.25 (m, 4H), 7.12-7.07 (m, 2H), 4.45-4.35 (m, 1H), 4.22-4.10 (m, 1H), 3.66-3.56 (m, 2H), 3.53-3.34 (m, 5H), 2.25-2.16 (m, 1H), 1.96-1.86 (m, 1H), 1.83-1.66 (m, 2H), 1.39 (s, 3H). MS-ESI calculated for [M+H]⁺ 491, found 491.

### Example 119

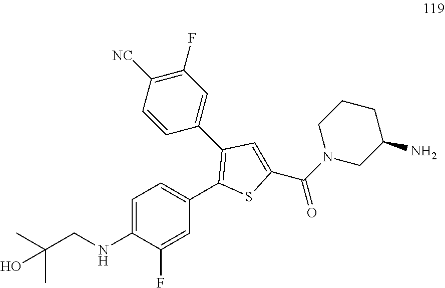 [SMILES: CC(C)(O)CNc1ccc(-c2sc(C(=O)N3CCC[C@@H](N)C3)cc2-c2ccc(C#N)c(F)c2)cc1F]

Synthetic Route:

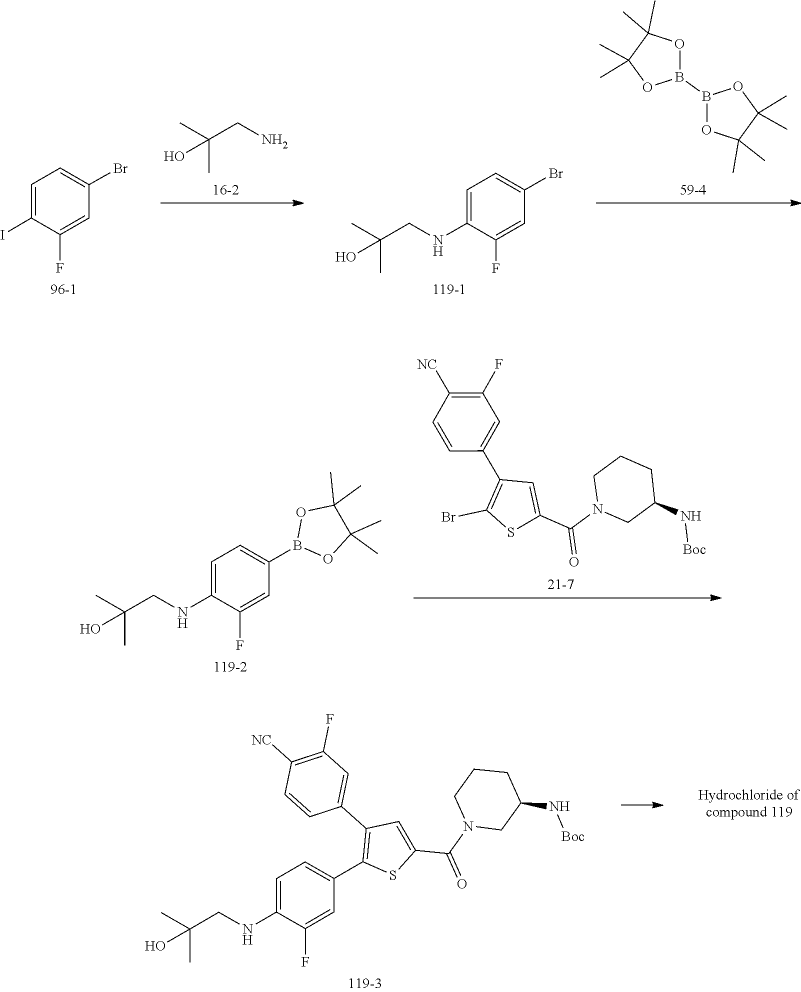

### Step 1

Compound 96-1 (7.00 g, 23.3 mmol) and compound 16-2 (4.15 g, 46.5 mmol) were dissolved in N,N-dimethylethanolamine (50.0 mL), and the reaction mixture was added with cuprous iodide (886 mg, 4.65 mmol) and potassium phosphate (9.88 g, 46.5 mmol). The reaction mixture was stirred and reacted for 12 hours at 55° C., added with saturated ammonium chloride solution (50 mL), and extracted with ethyl acetate (100 mL×1). The organic phase was washed with saturated brine (100 mL×1), dried over anhydrous sodium sulfate, filtered, and concentrated under reduced pressure. The residue was purified by silica gel column chromatography (6/1, petroleum ether/ethyl acetate, R_{f}=0.86) to obtain compound 119-1. MS-ESI calculated for [M+H]⁺ 264, found 264.

### Step 2

Under nitrogen atmosphere, compound 119-1 (1.00 g, 3.82 mmol), compound 59-4 (1.94 g, 7.63 mmol), and potassium acetate (749 mg, 7.63 mmol) were dissolved in dioxane (30 mL), and the reaction mixture was added with [1,1′-bis(diphenylphosphino)ferrocene]dichloropalladium(II) (279 mg, 0.381 mmol). The reaction mixture was stirred and reacted for 3 hours at 95° C., added with water (50 mL), and extracted with ethyl acetate (100 mL×1). The organic phase was washed with saturated brine (100 mL×1), dried over anhydrous sodium sulfate, filtered, and concentrated under reduced pressure. The residue was purified by silica gel column chromatography (1/1, petroleum ether/ethyl acetate, R_{f}=0.30) to obtain compound 119-2. MS-ESI calculated for [M+H]⁺ 310, found 310.

### Step 3

Under nitrogen atmosphere, compound 119-2 (1.00 g, 3.23 mmol), compound 21-7 (1.57 g, 3.08 mmol), and potassium acetate (605 mg, 6.16 mmol) were dissolved in dioxane (24.0 mL) and water (6.00 mL), and the reaction mixture was added with [1,1′-bis(diphenylphosphino)ferrocene]dichloropalladium(II) (225 mg, 0.308 mmol). The reaction mixture was stirred and reacted for 12 hours at 90° C., added with water (20 mL), and extracted with ethyl acetate (20 mL×1). The organic phase was washed with saturated brine (20 mL×1), dried over anhydrous sodium sulfate, filtered, and concentrated under reduced pressure. The residue was purified by silica gel column chromatography (1/1, petroleum ether/ethyl acetate, R_{f}=0.24) to obtain compound 119-3. MS-ESI calculated for [M+H]⁺ 611, found 611.

### Step 4

Compound 119-3 (1.2 g, 1.96 mmol) was dissolved in ethyl acetate (5.00 mL), and hydrochloride ethyl acetate solution (4 mol/L, 20 mL) was added thereto. The reaction mixture was stirred for 1 hour at 25° C., concentrated under reduced pressure, and purified by preparative high performance liquid chromatography (chromatographic column: Phenomenex C18 150*40 mm*5 μm; mobile phase: A: hydrochloric acid aqueous solution with a volume fraction of 0.05%; mobile phase B: acetonitrile 12% to 42%, 10 minutes) to obtain the hydrochloride of compound 119. ¹H NMR (400 MHz, CD₃OD) δ=7.74-7.66 (m, 1H), 7.55 (s, 1H), 7.37-7.27 (m, 2H), 7.07-6.99 (m, 3H), 4.42 (d, J=12.0 Hz, 1H), 4.23-4.08 (m, 1H), 3.56-3.36 (m, 3H), 3.22 (s, 2H), 2.30-2.14 (m, 1H), 1.90-1.94 (m, 1H), 1.86-1.64 (m, 2H), 1.30 (s, 6H). MS-ESI calculated for [M+H]⁺ 511, found 511.

### Example 120

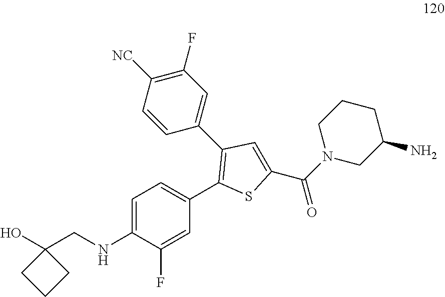 [SMILES: N#Cc1ccc(-c2cc(C(=O)N3CCC[C@@H](N)C3)sc2-c2ccc(NCC3(O)CCC3)c(F)c2)cc1F]

Synthetic Route:

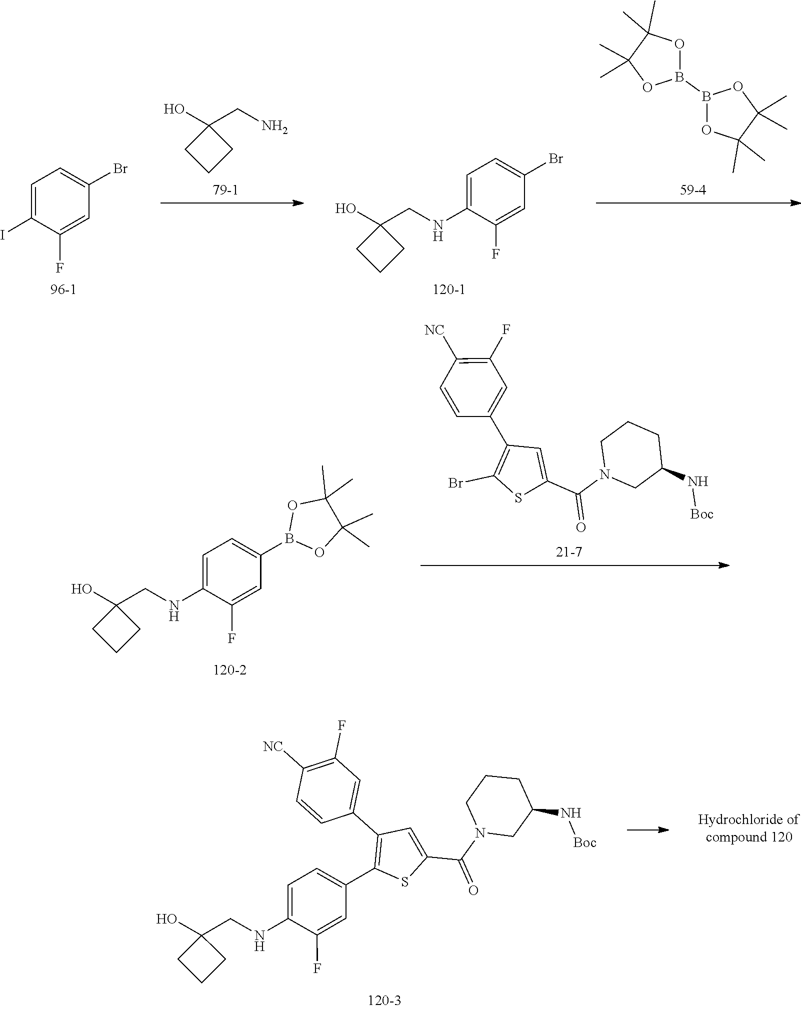

### Step 1

Compound 96-1 (7.00 g, 23.3 mmol) and compound 79-1 (4.71 g, 46.5 mmol) were dissolved in N,N-dimethylethanolamine (50.0 mL), and the reaction mixture was added with cuprous iodide (886 mg, 4.65 mmol) and potassium phosphate (9.88 g, 46.5 mmol). The reaction mixture was stirred and reacted for 12 hours at 55° C., added with saturated ammonium chloride solution (50 mL), and extracted with ethyl acetate (100 mL×1). The organic phase was washed with saturated brine (100 mL×1), dried over anhydrous sodium sulfate, filtered, and concentrated under reduced pressure. The residue was purified by silica gel column chromatography (6/1, petroleum ether/ethyl acetate, R_{f}=0.90) to obtain compound 120-1. MS-ESI calculated for [M+H]⁺ 276, found 276.

### Step 2

Under nitrogen atmosphere, compound 120-1 (1.00 g, 3.65 mmol), compound 59-4 (1.85 g, 7.30 mmol), and potassium acetate (1.07 mg, 10.9 mmol) were dissolved in dioxane (30.0 mL), and the reaction mixture was added with [1,1′-bis(diphenylphosphino)ferrocene]dichloropalladium(II) (263 mg, 0.365 mmol). The reaction mixture was stirred and reacted for 3 hours at 95° C., added with saturated ammonium chloride solution (50 mL), and extracted with ethyl acetate (100 mL×1). The organic phase was washed with saturated brine (100 mL×1), dried over anhydrous sodium sulfate, filtered, and concentrated under reduced pressure. The residue was purified by silica gel column chromatography (1/1, petroleum ether/ethyl acetate, R_{f}=0.70) to obtain compound 120-2. MS-ESI calculated for [M+H]⁺ 310, found 310.

### Step 3

Under nitrogen atmosphere, compound 120-2 (1.00 g, 3.11 mmol), compound 21-7 (1.51 g, 3.11 mmol), and potassium acetate (582 mg, 5.93 mmol) were dissolved in dioxane (24.0 mL) and water (6.00 mL), and the reaction mixture was added with [1,1′-bis(diphenylphosphino)ferrocene]dichloropalladium(II) (217 mg, 0.297 mmol). The reaction mixture was stirred and reacted for 12 hours at 95° C., added with water (20 mL), and extracted with ethyl acetate (20 mL×1). The organic phase was washed with saturated brine (20 mL×1), dried over anhydrous sodium sulfate, filtered, and concentrated under reduced pressure. The residue was purified by silica gel column chromatography (1/1, petroleum ether/ethyl acetate, R_{f}=0.24) to obtain compound 120-3. MS-ESI calculated for [M+H]⁺ 623, found 623.

### Step 4

Compound 120-3 (1.20 g, 1.96 mmol) was dissolved in ethyl acetate (5.00 mL), and hydrochloride ethyl acetate solution (4 mol/L, 20.0 mL) was added thereto. The reaction mixture was stirred for 2 hours at 25° C., concentrated under reduced pressure, and purified by preparative high performance liquid chromatography (chromatographic column: Phenomenex C18 150*40 mm*5 μm; mobile phase: A: hydrochloric acid aqueous solution with a volume fraction of 0.05%; mobile phase B: acetonitrile 12% to 42%, 10 minutes) to obtain the hydrochloride of compound 120. 1H NMR (400 MHz, CD3OD) δ=7.75-7.67 (m, 1H), 7.56 (s, 1H), 7.37-7.28 (m, 2H), 7.13-7.03 (m, 3H), 4.45-4.35 (m, 1H), 4.22-4.08 (m, 1H), 3.55-3.39 (m, 3H), 3.38 (s, 2H), 2.26-2.17 (m, 1H), 2.17-2.09 (m, 4H), 1.96-1.60 (m, 5H). MS-ESI calculated for [M+H]⁺ 523, found 523.

### Example 121

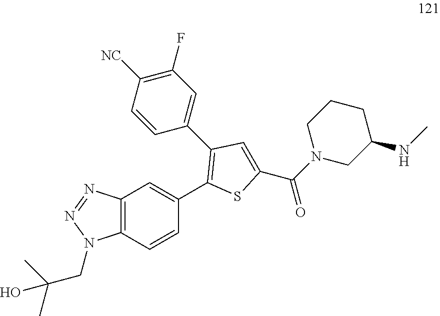 [SMILES: CN[C@@H]1CCCN(C(=O)c2cc(-c3ccc(C#N)c(F)c3)c(-c3ccc4c(c3)nnn4CC(C)(C)O)s2)C1]

Synthetic Route:

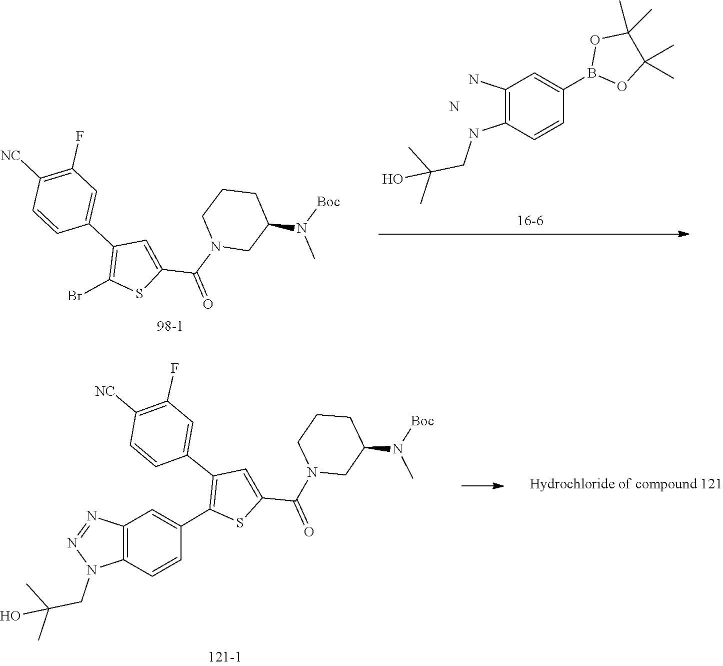

### Step 1

Under nitrogen atmosphere, compound 98-1 (800 mg, 1.53 mmol), compound 16-6 (583 mg, 1.84 mmol), and sodium carbonate (487 mg, 4.59 mmol) were dissolved in dioxane (8.00 mL) and water (2.00 mL), and the reaction mixture was added with [1,1′-bis(diphenylphosphino)ferrocene]dichloropalladium(II) (112 mg, 0.153 mmol). The reaction mixture was stirred and reacted for 2 hours at 80° C., added with water (20 mL), and extracted with ethyl acetate (20 mL×1). The organic phase was washed with saturated brine (20 mL×1), dried over anhydrous sodium sulfate, filtered, and concentrated under reduced pressure. The residue was purified by silica gel column chromatography (1/1, petroleum ether/ethyl acetate, R_{f}=0.21) to obtain compound 121-1. MS-ESI calculated for [M+H]⁺ 633, found 633.

### Step 2

Compound 121-1 (850 mg, 1.34 mmol) was dissolved in ethyl acetate (10.0 mL), and hydrochloride ethyl acetate solution (4 mol/L, 21.8 mL) was added thereto. The reaction mixture was stirred for 1 hour at 25° C., concentrated under reduced pressure, and purified by preparative high performance liquid chromatography (chromatographic column: Phenomenex Genimi NX C18 150*40 mm*5 μm; mobile phase: mobile phase A: hydrochloric acid aqueous solution with a volume fraction of 0.05%; mobile phase B: acetonitrile; B %: 10 to 40%, 10 minutes) to obtain the hydrochloride of compound 121. ¹H NMR (400 MHz, CD₃OD) δ 7.97 (s, 1H), 7.86 (d, J=8.8 Hz, 1H), 7.68-7.62 (m, 2H), 7.41 (dd, J=1.6, 8.8 Hz, 1H), 7.38-7.33 (m, 1H), 7.26 (dd, J=1.6, 8.8 Hz, 1H), 4.69 (s, 2H), 4.37-4.26 (m, 1H), 4.16-4.03 (m, 1H), 3.84-3.60 (m, 2H), 3.48-3.35 (m, 1H), 2.81 (s, 3H), 2.33-2.19 (m, 1H), 2.00-1.84 (m, 2H), 1.81-1.67 (m, 1H), 1.28 (s, 6H). MS-ESI calculated for [M+H]⁺ 533, found 533.

### Example 122

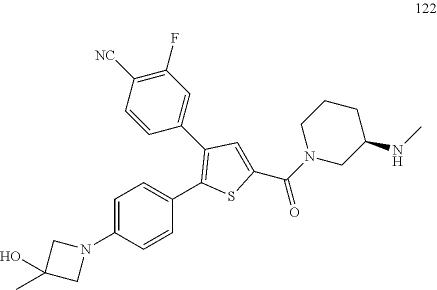

Synthetic Route:

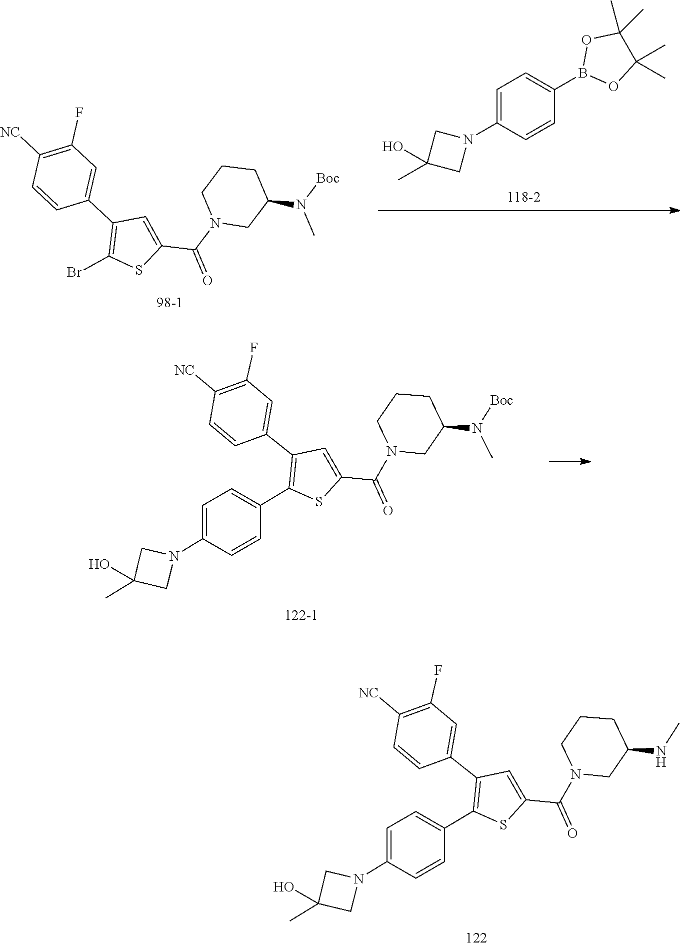

### Step 1

Under nitrogen atmosphere, compound 98-1 (780 mg, 1.34 mmol), compound 118-2 (475 mg, 1.64 mmol), and sodium carbonate (475 mg, 4.48 mmol) were dissolved in dioxane (8 mL) and water (2 mL), and the reaction mixture was added with [1,1′-bis(di-tert-butylphosphino)ferrocene]dichloropalladium(II) (97.3 mg, 0.149 mmol). The reaction mixture was stirred and reacted for 12 hours at 95° C., added with water (20 mL), and extracted with ethyl acetate (20 mL×1). The organic phase was washed with saturated brine (20 mL×1), dried over anhydrous sodium sulfate, filtered, and concentrated under reduced pressure. The residue was purified by silica gel column chromatography (1/1, petroleum ether/ethyl acetate, R_{f}=0.21) to obtain compound 122-1. MS-ESI calculated for [M+H]⁺ 605, found 605.

### Step 2

Compound 122-1 (700 mg, 1.16 mmol) was dissolved in ethyl acetate (10.0 mL), and hydrochloride ethyl acetate solution (4 mol/L, 10.0 mL) was added dropwise thereto at 25° C. The reaction mixture was stirred for 1 hour at 25° C., concentrated under reduced pressure, and purified by preparative high performance liquid chromatography (chromatographic column: Phenomenex C18 80*40 mm*3 μm; mobile phase: mobile phase A: ammonia aqueous solution with a volume fraction of 0.05%; mobile phase B: acetonitrile; B %: 44 to 47%, 8 minutes) to obtain compound 122. ¹H NMR (400 MHz, CD₃OD) δ 7.65 (t, J=7.6 Hz, 1H), 7.48 (s, 1H), 7.31-7.21 (m, 2H), 7.10 (d, J=8.4 Hz, 2H), 6.45 (d, J=8.4 Hz, 2H), 4.44-4.34 (m, 1H), 4.28-4.12 (m, 1H), 3.84 (d, J=7.6 Hz, 2H), 3.71 (d, J=7.6 Hz, 2H), 3.36-3.34 (m, 1H), 3.10-2.98 (m, 1H), 2.67-2.56 (m, 1H), 2.40 (s, 3H), 2.15-2.06 (m, 1H), 1.91-1.80 (m, 1H), 1.65-1.57 (m, 1H), 1.55 (s, 3H), 1.51-1.38 (m, 1H). MS-ESI calculated for [M+H]⁺ 505, found 505.

### Example 123

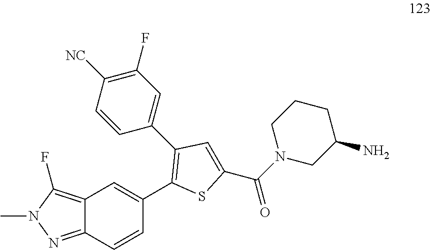 [SMILES: Cn1nc2ccc(-c3sc(C(=O)N4CCC[C@@H](N)C4)cc3-c3ccc(C#N)c(F)c3)cc2c1F]

Synthetic Route:

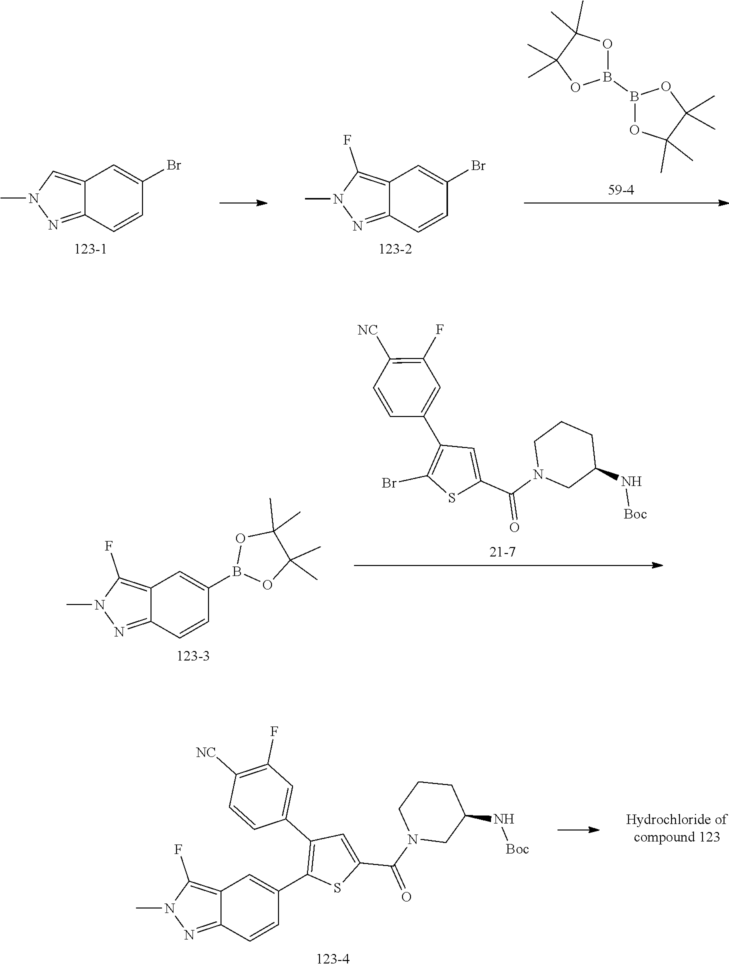

### Step 1

Compound 123-1 (3.00 g, 14.2 mmol) was dissolved in acetonitrile (100 mL), and the reaction mixture was added with 1-chloromethyl-4-fluoro-1,4-diazoniabicyclo[2.2.2]octane bis(tetrafluoroborate) (5.54 mg, 15.64 mmol). The reaction mixture was stirred and reacted for 12 hours at 25° C., added with water (50 mL), and extracted with ethyl acetate (100 mL×1). The organic phase was washed with saturated brine (100 mL×1), dried over anhydrous sodium sulfate, filtered, and concentrated under reduced pressure. The residue was purified by silica gel column chromatography (1/1, petroleum ether/ethyl acetate, R_{f}=0.70) to obtain compound 123-2. MS-ESI calculated for [M+H]⁺ 231, found 231. ¹H NMR (400 MHz, CD₃OD) δ=7.76 (d, J=1.2 Hz, 1H), 7.42-7.37 (m, 1H), 7.37-7.33 (m, 1H), 4.05 (d, J=2.0 Hz, 3H).

### Step 2

Under nitrogen atmosphere, compound 123-2 (625 mg, 2.73 mmol), compound 59-4 (1.39 g, 5.46 mmol), and potassium acetate (803 mg, 8.19 mmol) were dissolved in dioxane (30.0 mL), and the reaction mixture was added with [1,1′-bis(diphenylphosphino)ferrocene]dichloropalladium(II) (200 mg, 0.273 mmol). The reaction mixture was stirred and reacted for 12 hours at 100° C., added with water (20 mL), and extracted with ethyl acetate (20 mL×1). The organic phase was washed with saturated brine (20 mL×1), dried over anhydrous sodium sulfate, filtered, and concentrated under reduced pressure. The residue was purified by silica gel column chromatography (1/1, petroleum ether/ethyl acetate, R_{f}=0.60) to obtain compound 123-3. MS-ESI calculated for [M+H]⁺ 277, found 277.

### Step 3

Under nitrogen atmosphere, compound 123-3 (600 mg, 2.14 mmol), compound 21-7 (1.00 g, 1.82 mmol), and potassium acetate (388 mg, 3.95 mmol) were dissolved in dioxane (12.0 mL) and water (3.00 mL), and the reaction mixture was added with [1,1′-bis(diphenylphosphino)ferrocene]dichloropalladium(II) (144 mg, 0.190 mmol). The reaction mixture was stirred and reacted for 3 hours at 100° C., added with water (20 mL), and extracted with ethyl acetate (20 mL×1). The organic phase was washed with saturated brine (20 mL×1), dried over anhydrous sodium sulfate, filtered, and concentrated under reduced pressure. The residue was purified by silica gel column chromatography (1/1, petroleum ether/ethyl acetate, R_{f}=0.36) to obtain compound 123-4. MS-ESI calculated for [M+H]⁺ 623, found 623.

### Step 4

Compound 123-4 (900 mg, 1.56 mmol) was dissolved in ethyl acetate (10.0 mL), and hydrochloride ethyl acetate solution (4 mol/L, 18.6 mL) was added dropwise thereto at 25° C. The reaction mixture was stirred for 1 hour at 25° C., concentrated under reduced pressure, and purified by preparative high performance liquid chromatography (chromatographic column: Xtimate C18 150*40 mm*5 μm; mobile phase: A: hydrochloric acid aqueous solution with a volume fraction of 0.05%; mobile phase B: acetonitrile 18% to 48%, 10 minutes) to obtain the hydrochloride of compound 123. ¹H NMR (400 MHz, CD₃OD) δ=7.70-7.60 (m, 3H), 7.44 (d, J=9.2 Hz, 1H), 7.33 (d, J=10.4 Hz, 1H), 7.28 (d, J=8.0 Hz, 1H), 7.12 (d, J=9.2 Hz, 1H), 4.50-4.37 (m, 1H), 4.20-4.14 (m, 1H), 4.08 (s, 3H), 3.56-3.40 (m, 3H), 2.28-2.16 (m, 1H), 2.00-1.67 (m, 3H). MS-ESI calculated for [M+H]⁺ 478, found 478.

Biochemical Assays:

Experiment 1A: Evaluation of Enzyme Activity

Testing unit: Wuhan Heyan Biomedical Technology Co., Ltd.

The purpose of the assay was to test the in vitro inhibitory activity of the compounds against LSD1. The enzyme used in the assay was human LSD1, and the standard substrate was histone H3K4me peptide (20 μM). The activity of the compounds was determined by the enzyme-coupling fluorescent method by the combined detection of H₂O₂ generated after the reaction of LSD1 by horseradish peroxidase (HRP) and the fluorescent reagent Amplex Red. Starting from 10 μM, the compounds were 3-fold diluted to detect the IC₅₀ values of the compounds at 10 concentrations. The enzyme and substrate were incubated for 30 minutes before the compound was added to the substrate to start the reaction. Fluorescence detector: EnVision, excitation wavelength: Ex/Em=530/590 nM.

The compounds were tested for LSD1 inhibitory activity, and the results are shown in Table 1.

TABLE 1
In vitro enzyme activity screening test results
of compounds of the present disclosure

| Compound No. | IC₅₀ (nM) |
|---|---|
| Hydrochloride of compound 1 | 81.96 |
| Hydrochloride of compound 2 | 122.4 |
| Hydrochloride of compound 4 | 109.8 |
| Hydrochloride of compound 5 | 344.9 |
| Hydrochloride of compound 6 | 111.9 |
| Hydrochloride of compound 7 | 120.7 |
| Hydrochloride of compound 8 | 120.5 |
| Hydrochloride of compound 9 | 117.6 |
| Hydrochloride of compound 10 | 192.3 |
| Hydrochloride of compound 12 | 393.2 |
| Hydrochloride of compound 15 | 310.3 |

Experiment 1B: Evaluation of Enzyme Activity

Testing unit: Reaction Biology Corp., USA.

The purpose of the assay was to test the in vitro inhibitory activity of the compounds against LSD1. The enzyme used in the assay was human LSD1, and the standard substrate was histone H3(1-21)K4me2 peptide (10 μM). The activity of the compounds was determined by the enzyme-coupling fluorescent method by the combined detection of H₂O₂ generated after the reaction of LSD1 by horseradish peroxidase (HRP) and the fluorescent reagent Amplex Red. Starting from 10 μM, the compounds were 3-fold diluted to detect the IC₅₀ values of the compounds at 10 concentrations. The enzyme and substrate were incubated for 30 minutes before the compound was added to the substrate to start the reaction. Fluorescence detector: EnVision, excitation wavelength: Ex/Em=535/590 nM.

The compounds were tested for LSD1 inhibitory activity, and the results are shown in Table 2.

TABLE 2
In vitro enzyme activity screening test results
of compounds of the present disclosure

| Compound No. | IC₅₀ (nM) |
|---|---|
| Compound 14 | 5.37 |
| Hydrochloride of compound 16 | 7.51 |
| Hydrochloride of compound 18 | 6.60 |
| Hydrochloride of compound 19 | 64.8 |
| Hydrochloride of compound 20 | 19.4 |
| Hydrochloride of compound 21 | 19.4 |
| Hydrochloride of compound 23 | 76.8 |
| Hydrochloride of compound 24 | 70.9 |
| Hydrochloride of compound 25 | 63.9 |
| Hydrochloride of compound 26 | 19.6 |
| Hydrochloride of compound 27 | 48.2 |
| Hydrochloride of compound 29 | 29.7 |
| Hydrochloride of compound 30 | 122 |
| Hydrochloride of compound 31 | 26.7 |
| Hydrochloride of compound 33 | 38.0 |
| Hydrochloride of compound 34 | 36.5 |
| Hydrochloride of compound 35 | 24.6 |
| Hydrochloride of compound 36 | 3.44 |
| Hydrochloride of compound 37 | 1.73 |
| Compound 40 | 95.6 |
| Hydrochloride of compound 44 | 70.6 |
| Hydrochloride of compound 46 | 81.3 |
| Hydrochloride of compound 47 | 48.8 |
| Hydrochloride of compound 49 | 101 |
| Compound 50 | 28.3 |
| Hydrochloride of compound 52 | 251 |
| Hydrochloride of compound 54 | 49.9 |
| Hydrochloride of compound 56 | 109 |
| Compound 57 | 10.4 |
| Compound 58 | 20.4 |
| Compound 59 | 43.1 |
| Compound 60 | 18.4 |
| Hydrochloride of compound 62 | 30.1 |
| Hydrochloride of compound 64 | 29.6 |
| Hydrochloride of compound 66 | 73.4 |
| Hydrochloride of compound 72 | 8.56 |
| Hydrochloride of compound 73 | 11.8 |
| Compound 74 | 33.2 |
| Compound 75 | 28.9 |
| Compound 78 | 47.4 |
| Hydrochloride of compound 79 | 13.6 |
| Hydrochloride of compound 80 | 20.2 |
| Hydrochloride of compound 82 | 3.01 |
| Compound 84 | 13.2 |
| Hydrochloride of compound 85 | 14.2 |
| Hydrochloride of compound 86 | 16.0 |
| Hydrochloride of compound 87 | 24.8 |
| Hydrochloride of compound 88 | 87.6 |
| Hydrochloride of compound 89 | 15.2 |
| Hydrochloride of compound 90 | 37.3 |
| Hydrochloride of compound 91 | 13.0 |
| Hydrochloride of compound 92 | 9.81 |
| Hydrochloride of compound 93 | 18.0 |
| Hydrochloride of compound 94 | 98.4 |
| Hydrochloride of compound 95 | 10.1 |
| Hydrochloride of compound 96 | 20.6 |
| Hydrochloride of compound 97 | 25.3 |
| Hydrochloride of compound 98 | 12.6 |
| Hydrochloride of compound 99 | 17.4 |
| Hydrochloride of compound 100 | 14.1 |
| Hydrochloride of compound 101 | 28.7 |
| Hydrochloride of compound 102 | 56.9 |
| Hydrochloride of compound 103 | 21.6 |
| Hydrochloride of compound 104 | 16.6 |
| Hydrochloride of compound 107 | 15.1 |
| Hydrochloride of compound 109 | 10.7 |
| Hydrochloride of compound 110 | 9.84 |
| Hydrochloride of compound 111 | 17.6 |
| Hydrochloride of compound 112 | 5.84 |
| Hydrochloride of compound 116 | 9.27 |
| Hydrochloride of compound 117 | 19.0 |
| Hydrochloride of compound 118 | 13.8 |
| Hydrochloride of compound 119 | 17.7 |
| Hydrochloride of compound 120 | 9.4 |
| Hydrochloride of compound 121 | 14.3 |
| Compound 122 | 13.1 |
| Hydrochloride of compound 123 | 26.7 |

Conclusion: The compounds of the present disclosure exhibit significant inhibitory activity against LSD1.

Experiment 2: Evaluation of Inhibitory Activity on NCI-H1417 Cell Proliferation

Experimental purpose: To test the inhibitory activity of test compounds on NCI-H1417 cell proliferation.

Experimental materials: RPMI 1640 medium, fetal bovine serum, Promega CellTiter-Glo reagent. The NCI-H1417 cell line was purchased from ATCC. Envision multilabel plate reader (PerkinElmer).

Experimental method: The compounds were dissolved to 10 mM and 5-fold diluted with DMSO in the compound plates, starting with a concentration of 2 mM. 3-fold serial dilution was performed with Bravo to 10 concentrations. Then 250 nL of the compound solution was transferred to the upper and lower double duplicate wells of a blank 384 cell plate with Echo. To each well containing the transferred 250 nL of DMSO/compound was added a cell suspension of 1000 cells/50 μL per well, and the compound was diluted 200-fold, i.e., resulting in an initial working concentration of 10 μM. The cell plate was incubated in a carbon dioxide incubator for 10 days. To the cell plate was added 25 μL of Promega CellTiter-Glo reagent per well and the mixture was shaken for 10 minutes at room temperature to stabilize the luminescence signal. Readings were performed on a PerkinElmer Envision multilabel plate reader.

Data analysis: Raw data were converted into inhibition rate using the formula: (Max−Ratio)/(Max−Min)*100%. The IC₅₀ values could then be derived from four-parameter curve fitting. (derived from 205 mode in XLFIT5, iDBS)

The compounds were tested for inhibitory activity of compounds on NCI-H1417 cell proliferation, and the results are shown in Table 3.

TABLE 3
Results of the inhibition assay of compounds of the
present disclosure on NCI-H1417 cell proliferation

| Compound No. | IC₅₀ (nM) |
|---|---|
| Hydrochloride of compound 1 | 6.88 |
| Hydrochloride of compound 2 | 1.75 |
| Hydrochloride of compound 4 | 30.15 |
| Hydrochloride of compound 6 | 37.89 |
| Hydrochloride of compound 7 | 20.41 |
| Hydrochloride of compound 8 | 8.73 |
| Hydrochloride of compound 9 | 15.82 |
| Hydrochloride of compound 10 | 22.41 |
| Compound 14 | 0.54 |
| Hydrochloride of compound 16 | 0.17 |
| Hydrochloride of compound 18 | 18.16 |
| Hydrochloride of compound 20 | 32.31 |
| Hydrochloride of compound 21 | 6.83 |
| Hydrochloride of compound 24 | 30.62 |
| Hydrochloride of compound 26 | 6.87 |
| Hydrochloride of compound 29 | 17.17 |
| Hydrochloride of compound 31 | 11.27 |
| Hydrochloride of compound 33 | 16.21 |
| Hydrochloride of compound 34 | 9.92 |
| Hydrochloride of compound 35 | 8.84 |
| Hydrochloride of compound 36 | 1.17 |
| Hydrochloride of compound 37 | 0.93 |
| Compound 40 | 18.68 |
| Hydrochloride of compound 44 | 8.87 |
| Hydrochloride of compound 46 | 19.40 |
| Hydrochloride of compound 47 | 10.19 |
| Compound 50 | 9.19 |
| Hydrochloride of compound 54 | 5.11 |
| Hydrochloride of compound 56 | 56.79 |
| Compound 57 | 1.53 |
| Compound 58 | 3.56 |
| Compound 59 | 13.2 |
| Compound 60 | 2.36 |
| Hydrochloride of compound 62 | 11.18 |
| Hydrochloride of compound 63 | 28.57 |
| Hydrochloride of compound 64 | 16.16 |
| Hydrochloride of compound 65 | 11.05 |
| Compound 70 | 9.88 |
| Hydrochloride of compound 71 | 0.64 |
| Hydrochloride of compound 72 | 4.47 |
| Hydrochloride of compound 73 | 14.46 |
| Compound 75 | 28.0 |
| Hydrochloride of compound 79 | 3.32 |
| Hydrochloride of compound 80 | 16.14 |
| Hydrochloride of compound 82 | 1.73 |
| Compound 84 | 25.93 |
| Hydrochloride of compound 85 | 2.32 |
| Hydrochloride of compound 86 | 10.29 |
| Hydrochloride of compound 87 | 5.11 |
| Hydrochloride of compound 88 | 39.35 |
| Hydrochloride of compound 89 | 1.61 |
| Hydrochloride of compound 90 | 37.57 |
| Hydrochloride of compound 91 | 0.83 |
| Hydrochloride of compound 92 | 0.43 |
| Hydrochloride of compound 93 | 4.11 |
| Hydrochloride of compound 94 | 32.78 |
| Hydrochloride of compound 95 | 2.51 |
| Hydrochloride of compound 96 | 4.79 |
| Hydrochloride of compound 97 | 9.60 |
| Hydrochloride of compound 98 | 0.07 |
| Hydrochloride of compound 99 | 4.26 |
| Hydrochloride of compound 100 | 0.37 |
| Hydrochloride of compound 101 | 3.35 |
| Hydrochloride of compound 102 | 31.41 |
| Hydrochloride of compound 103 | 13.66 |
| Hydrochloride of compound 104 | 4.75 |
| Hydrochloride of compound 105 | 6.14 |
| Hydrochloride of compound 106 | 0.19 |
| Hydrochloride of compound 107 | 3.31 |
| Hydrochloride of compound 108 | 2.11 |
| Hydrochloride of compound 109 | 2.03 |
| Hydrochloride of compound 110 | 3.47 |
| Hydrochloride of compound 111 | 2.12 |
| Hydrochloride of compound 112 | 4.99 |
| Hydrochloride of compound 113 | 3.98 |
| Hydrochloride of compound 114 | 5.18 |
| Hydrochloride of compound 115 | 1.94 |
| Hydrochloride of compound 117 | 40.17 |
| Hydrochloride of compound 118 | 31.58 |
| Hydrochloride of compound 119 | 9.17 |
| Hydrochloride of compound 120 | 17.46 |
| Hydrochloride of compound 121 | 7.7 |
| Compound 122 | 9.83 |
| Hydrochloride of compound 123 | 6.25 |

Conclusion: The compounds of the present disclosure have significant inhibitory activity on NCI-H1417 cell proliferation.

Experiment 3: Pharmacokinetic Evaluation of Compounds

Experimental purpose: To test the pharmacokinetics of compounds in CD-1 mice in vivo

Experimental Materials:

CD-1 mice (male/female, 7 to 9 weeks old, Shanghai SLAC)

Experimental Operation:

The pharmacokinetic characteristics of the compounds in rodents after intravenous injection and oral administration were tested according to the standard protocol. In the experiment, the candidate compound was formulated into clear solutions, and a single intravenous injection and oral administration were given to mice. The vehicle for intravenous injection and oral administration was a mixed vehicle composed of 10% dimethyl sulfoxide and 90% hydroxypropyl β-cyclodextrin with a content of 10%. In this project, four male/female CD-1 mice were used. Two mice were injected intravenously at a dose of 1 mg/kg, and plasma samples were collected at 0.083, 0.25, 0.5, 1, 2, 4, 8, and 24 hours after administration; the other two were administered orally by gavage at a dose of 2 mg/kg, and plasma samples were collected at 0.25, 0.5, 1, 2, 4, 8, and 24 hours after administration. The samples were centrifuged at 3200 g for 10 minutes at 4° C., and plasma samples were obtained by separating the supernatant. A methanol solution containing internal standard with 20 times the volume of the sample was added thereto to precipitate the protein, and the mixture was centrifuged at 12000 g for 15 minutes at 4° C. 50 μL of the supernatant was taken and transferred to a 96-well plate and centrifuged again. The supernatant was taken for sampling. The plasma concentration was quantitatively analyzed using LC-MS/MS analysis method, and pharmacokinetic parameters, such as peak concentration (Cₘₐₓ), clearance rate (CL), half-life (T_{1/2}), volume of distribution at steady state (Vdss), area under drug-time curve (AUC₀₋ₗₐₛₜ), bioavailability (F).

The experimental results are shown in Table 4 and Table 5:

TABLE 4
Pharmacokinetic test results in female CD-1 mice

| | | | | | Area | |
|---|---|---|---|---|---|---|
| | | Clearance | | | under | |
| | | rate | Volume of | Half | drug-time | |
| | Peak | CL | distribution | life | curve | |
| | concentration | (mL/ | at steady | T_{1/2} | AUC₀₋ₗₐₛₜ | |
| | Cₘₐₓ | min/ | state Vdss | (IV, | PO (nM · | Bioavailability |
| Compound | (nM) | kg) | (L/kg) | h) | hr) | F (%) |
| Hydrochloride | 245 | 17.8 | 7.66 | 5.59 | 1562 | 47.2 |
| of compound | | | | | | |
| 1* | | | | | | |
| Hydrochloride | 209 | 19.6 | 7.90 | 5.23 | 1428 | 47.7 |
| of compound | | | | | | |
| 2* | | | | | | |
| Hydrochloride | 222 | 18.0 | 7.76 | 5.37 | 2519 | 64.0 |
| of compound 6 | | | | | | |
| Hydrochloride | 176 | 20.9 | 6.19 | 3.74 | 1886 | 52.4 |
| of compound 7 | | | | | | |
| Hydrochloride | 224 | 37.3 | 4.71 | 2.01 | 971 | 49.6 |
| of compound | | | | | | |
| 36 | | | | | | |

*Note:
The sampling time points for intravenous injection of the hydrochloride of compound 1 and the hydrochloride of compound 2 are 0.033, 0.083, 0.25, 0.5, 1, 2, 4, 8, and 12 hours, and the sampling time points for oral gavage are 0.083, 0.25, 0.5, 1, 2, 4, 8, and 12 hours. The sampling time points of administration of other compounds were performed according to the above experimental protocol.

TABLE 5
Pharmacokinetic test results in male CD-1 mice

| | | | | | Area | |
|---|---|---|---|---|---|---|
| | | Clearance | | | under | |
| | | rate | Volume of | Half | drug-time | |
| | Peak | CL | distribution | life | curve | |
| | concentration | (mL/ | at steady | T_{1/2} | AUC₀₋ₗₐₛₜ | |
| | Cₘₐₓ | min/ | state Vdss | (IV, | PO (nM · | Bioavailability |
| Compound | (nM) | kg) | (L/kg) | h) | hr) | F (%) |
| Hydrochloride | 176 | 16.7 | 8.33 | 6.13 | 2241 | 53.9 |
| of compound 1 | | | | | | |
| Hydrochloride | 148 | 31.5 | 4.47 | 2.1 | 742 | 33.9 |
| of compound | | | | | | |
| 36 | | | | | | |
| Hydrochloride | 109 | 33.7 | 4.89 | 1.93 | 576 | 31.9 |
| of compound | | | | | | |
| 80 | | | | | | |
| Hydrochloride | 267 | 25.7 | 3.13 | 1.54 | 1012 | 41.1 |
| of compound | | | | | | |
| 87 | | | | | | |
| Hydrochloride | 160 | 45.3 | 6.94 | 2.28 | 708 | 58.6 |
| of compound | | | | | | |
| 98 | | | | | | |
| Hydrochloride | 145 | 42.0 | 7.23 | 2.48 | 625 | 47.7 |
| of compound | | | | | | |
| 99 | | | | | | |

Conclusion: The compounds of the present disclosure have good pharmacokinetic properties, including good oral bioavailability, oral exposure, half-life, clearance rate, etc.

Experiment 4: hERG Potassium Ion Channel Inhibition Assay

Experimental purpose: To test the effects of the test examples on hERG potassium ion channels using an automated patch clamp method.

Experimental Methods

4.1 Cell Preparation

4.1.1 CHO-hERG cells were cultured in a 175 cm² culture flask. When the cell density grew from 60% to 80%, the culture medium was removed. The cells were washed once with 7 mL of PBS (Phosphate Buffered Saline), and then 3 mL of Detachin cell dissociation reagent was added thereto for digestion.

4.1.2 After the digestion was completed, 7 mL of culture medium was added thereto for neutralization, then the mixture was centrifuged, the supernatant was aspirated, and then 5 mL of culture medium was added thereto for resuspension to ensure a cell density of 2×10⁶/mL to 5×10⁶/ML.

4.2 Solution preparation, and the composition of intracellular fluid and extracellular fluid are shown in Table 6.

TABLE 6
Composition of intracellular fluid and extracellular fluid

| Reagents | Extracellular fluid (mM) | Intracellular fluid (mM) |
|---|---|---|
| CaCl₂ | 1 | 1 |
| MgCl₂ | 1.25 | 1 |
| KCl | 5 | 140 |
| NaCl | 140 | 0 |
| Glucose | 10 | 0 |
| HEPES | 10 | 10 |
| EGTA | 0 | 10 |
| pH | Adjust pH to 7.4 with | Adjust pH to 7.2 with |
| | NaOH | KOH |
| Osmotic pressure | 305 mOsm | 290 mOsm |

4.3 Electrophysiological Recording Process

The single-cell high-resistance sealing and whole-cell modeling formation processes were all automatically completed by the Qpatch instrument of the Shanghai Institute of Materia Medica, Chinese Academy of Sciences. After obtaining the whole-cell recording modeling, the cells were clamped at −80 mV, and before a 5-second +40 mV depolarizing stimulus was given, a 50-millisecond −50 mV prepulse was first given, then the voltage was repolarized to −50 mV and holden for 5 seconds, and finally returned to −80 mV. This voltage stimulus was applied every 15 seconds, recorded for 2 minutes, and then the extracellular fluid was recorded for 5 minutes, and then the administration process was initiated, with compound concentrations starting at the lowest test concentration. The voltage stimulus was given for 2.5 minutes for each test concentration, and after all concentrations had been continuously given, 3 μM the positive control compound, Cisapride, was given. At least 3 cells were tested per concentration (n≥3).

4.4 Compound Preparation

4.4.1 The compound stock solution was diluted with DMSO, and 20 μL of DMSO solution was added with 10 μL of the compound stock solution, and serially diluted 3-fold to 6 DMSO concentrations.

4.4.2 4 μL of 6 DMSO concentrations of compounds were taken respectively and added to 396 μL of extracellular fluid, and the mixture was diluted 100-fold to 6 intermediate concentrations, then 80 μL of 6 intermediate concentrations of compounds was taken and added to 320 μL of extracellular fluid, and the mixture was diluted 5-fold to the final test concentration.

4.4.3 The highest test concentration was 40 μM, and the concentrations were respectively 40, 13.3, 4.4, 1.48, 0.494, and 0.165 μM for a total of six concentrations.

4.4.4 The DMSO content in the final test concentration did not exceed 0.2%, and this concentration of DMSO did not affect the hERG potassium channel.

4.4.5 The preparation of the compound was completed by the Bravo instrument throughout the dilution process.

4.5 Data Analysis

Experimental data were analyzed by GraphPad Prism 5.0 software.

4.6 The test results are shown in Table 7.

TABLE 7
Results of hERG IC50 values for compounds
of the present disclosure

| Compound No. | hERG IC₅₀ (μM) |
|---|---|
| Hydrochloride of compound 16 | >40 |
| Hydrochloride of compound 30 | >40 |
| Hydrochloride of compound 32 | >40 |
| Hydrochloride of compound 45 | 32.6 |
| Hydrochloride of compound 46 | >40 |
| Hydrochloride of compound 54 | >40 |
| Hydrochloride of compound 56 | >40 |
| Compound 59 | >40 |
| Compound 61 | >40 |
| Hydrochloride of compound 62 | >40 |
| Compound 67 | >40 |
| Compound 70 | 18.7 |
| Hydrochloride of compound 73 | >40 |
| Compound 76 | >40 |
| Compound 77 | >40 |
| Compound 81 | >40 |
| Hydrochloride of compound 82 | 14.9 |
| Hydrochloride of compound 86 | 21 |
| Hydrochloride of compound 87 | 13.4 |
| Hydrochloride of compound 88 | 21.9 |
| Hydrochloride of compound 89 | 22.7 |
| Hydrochloride of compound 90 | >40 |
| Hydrochloride of compound 91 | >40 |
| Hydrochloride of compound 94 | >40 |
| Hydrochloride of compound 98 | 18.6 |
| Hydrochloride of compound 99 | 27 |
| Hydrochloride of compound 111 | 21.5 |
| Hydrochloride of compound 121 | 20.5 |

Conclusion: The compounds of the present disclosure exhibit no significant inhibitory effect on hERG potassium ion channels.

Experiment 5: In Vivo Pharmacodynamic Study of Compounds on CB17-SCID Mouse Model of Human Small Cell Lung Cancer NCI-H1417 Cell Subcutaneous Xenograft Tumor

5.1 Experimental Purpose:

The purpose of this experiment was to evaluate the in vivo efficacy of the compounds of the present disclosure on CB17-SCID mouse model of human small cell lung cancer NCI-H1417 cell subcutaneous xenograft tumor.

5.2 Experimental Animals:
Species: mice
Strain: CB17-SCID mice
Week age and weight: 6-8 weeks old, 18 to 22 grams
Gender: Female
Supplier: Beijing Vital River Laboratory Animal Technology Co., Ltd.

5.3 Experimental Methods and Steps

5.3.1 Cell Culture

Human lung cancer cells NCI-H1417 were cultured in vitro in suspension in RPMI 1640 medium with 10% fetal bovine serum, 100 U/mL penicillin, and 100 μg/mL streptomycin at 37° C. in a 5% CO₂ cell culture incubator. Routine digestion and passage treatment were performed twice a week. When the number of cells reached the required number, the cells were counted and resuspended in PBS at a density of 10×10⁶ cells/200 μL.

5.3.2 Tumor Cell Inoculation

0.2 mL of NCI-H1417 tumor cells (containing 10×10⁶ cells) suspension (PBS: matrix gel=1:1) was subcutaneously inoculated on the right back of mice, and in pharmacodynamic experiments, on the 16th day after cell inoculation, when the average tumor volume reached 86 mm³, the subjects were randomly grouped and the administration started.

5.3.3 Preparation of Test Substance

The experimental vehicle was 5% dimethyl sulfoxide/95% (10% Solutol) solution. Preparation method: 3.8 mL of PEG400 was taken and added to 34.2 mL of double-distilled water, 2 mL of dimethyl sulfoxide was added thereto, and the mixture was mixed well. The test substance was dissolved with the vehicle, prepared as a homogeneous solution of a certain concentration, and stored at 4° C.

5.3.4 Tumor Measurement and Experimental Indicators

The experimental indicator was to examine whether tumor growth was inhibited, delayed, or cured. The tumor diameter was measured twice a week using vernier calipers. The calculation formula of tumor volume is V=0.5a×b², wherein a and b represent the long and short diameters of the tumor respectively.

The tumor inhibitory efficacy of compounds was evaluated by TGI (%). TGI (%) reflects the tumor growth inhibition rate. Calculation of TGI (%): TGI (%)=[1−(Average tumor volume at the end of administration in a certain treatment group−Average tumor volume at the beginning of administration in this treatment group)/(Average tumor volume at the end of treatment in the vehicle control group−Average tumor volume at the beginning of treatment in the vehicle control group)]×100%.

5.4 The experimental results are shown in Table 8.

TABLE 8
Evaluation of tumor inhibitory efficacy of compounds
on human small cell lung cancer NCI-H1417 cell subcutaneous
xenograft tumor model (calculated based on tumor
volume at day 28 after administration)

| | Mean tumor | |
|---|---|---|
| | volume ± SEM | TGI |
| Group | (mm³) (Day 28) | (%) |
| Vehicle group 5% DMSO/95% (10% | | / |
| polyoxyl 15 hydroxystearate aqueous | 446 ± 50 | |
| solution) | | |
| Hydrochloride of compound 36 | 87 ± 11 | 99.65 |
| (10 mg/kg, p.o., QD) | | |
| Hydrochloride of compound 36 | 53 ± 3 | 109.13 |
| (10 mg/kg, p.o., BID) | | |
| Hydrochloride of compound 87 | 67 ± 5 | 105.17 |
| (30 mg/kg, p.o., BID) | | |
| Hydrochloride of compound 98 | 44 ± 2 | 111.58 |
| (5 mg/kg, p.o., QD) | | |
| Hydrochloride of compound 98 | 34 ± 1 | 114.20 |
| (5 mg/kg, p.o., BID) | | |
| Hydrochloride of compound 98 | 38 ± 3 | 113.35 |
| (10 mg/kg, p.o., QD) | | |
| Hydrochloride of compound 121 | 75 ± 4 | 103.19 |
| (10 mg/kg, p.o., QD) | | |

Conclusion: The compounds of the present disclosure have excellent tumor inhibitory effect on the human small cell lung cancer NCI-H1417 xenograft tumor model.

1. A compound of formula (II) or a pharmaceutically acceptable salt thereof, 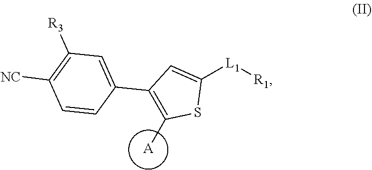
wherein L₁ is —C(═O)— or —C(═O)—NH—;
R₁ is R₁ₐ or 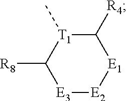
ring A is 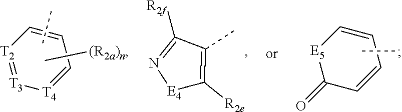
T₁ is CH or N;
T₂ is CR_{2c} or N;
T₃ is CR₂ or N;
T₄ is CR_{2b} or N;
E₁ is a single bond, —C(R₅)₂—, or —C(R₅)₂C(R₅)₂—;
E₂ is —NR₆— or —C(R₆₁)₂—;
E₃ is a single bond, —C(R₇)₂—, or —C(R₇)₂C(R₇)₂—;
E₄ is O, S, CH₂, or NR_{2g};
E₅ is O, CH₂, or NR_{2g};
R₁ₐ is C₁₋₃ alkyl substituted by 1, 2, or 3 Rₐ;
R₂ₐ is H or halogen;
R₂ and R_{2b} are each independently H, halogen, NH₂, C₁₋₃ alkyl, C₁₋₃ alkoxy, —S(═O)₂C₁₋₃ alkyl, —NHC₁₋₃ alkyl, 4- to 6-membered heterocycloalkyl, —C(═O)-4- to 6-membered heterocycloalkyl, or —NHC₁₋₃ alkyl-C₃₋₆ cycloalkyl, wherein the C₁₋₃ alkyl, C₁-3 alkoxy, —S(═O)₂C₁₋₃ alkyl, —NHC₁₋₃ alkyl, 4- to 6-membered heterocycloalkyl, —C(═O)-4- to 6-membered heterocycloalkyl, and —NHC₁₋₃ alkyl-C₃₋₆ cycloalkyl are each independently and optionally substituted by 1, 2, or 3 R_{b};
R_{2c} is H;
or, R₂ and R_{2c}, together with the C atom to which they are attached, make the structural moiety 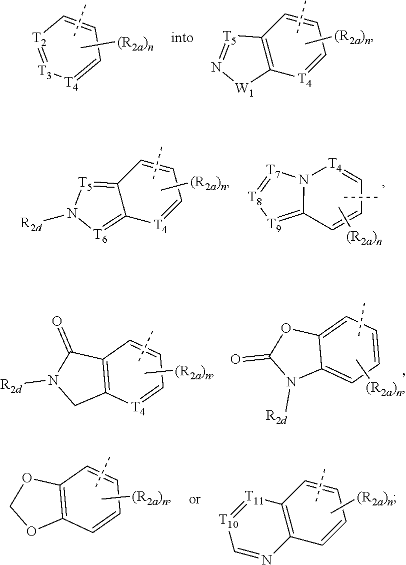
T₅, T₆, T₇, T₈, T₉, T₁₀, and T₁₁ are each independently N or CRₜ;
W₁ is O or NR_{2d};
R_{2d} is H, C₁₋₄ alkyl, C₃₋₆ cycloalkyl, 4- to 6-membered heterocycloalkyl, —C₁₋₃ alkyl-C₃₋₆ cycloalkyl, or —C₁₋₃ alkyl-4- to 6-membered heterocycloalkyl, wherein the C₁₋₄ alkyl, C₃₋₆ cycloalkyl, 4- to 6-membered heterocycloalkyl, —C₁₋₃ alkyl-C₃₋₆ cycloalkyl, and —C₁₋₃ alkyl-4- to 6-membered heterocycloalkyl are each independently and optionally substituted by 1, 2, or 3 R_{c};
R₂ₑ, R_{2f}, and R_{2g} are each independently H or C₁₋₃ alkyl;
each R₃ is independently H, halogen, or C₁₋₃ alkoxy;
R₄ and R₈ are each independently H or C₁₋₃ alkyl;
each R₅ is independently H, halogen, OH, NH₂, C₁₋₃ alkyl, —NHC₁₋₃ alkyl, or —N(C₁₋₃ alkyl)₂, wherein the C₁₋₃ alkyl, —NHC₁₋₃ alkyl, and —N(C₁₋₃ alkyl)₂ are each independently and optionally substituted by 1, 2, or 3 R_{d};
R₆ is H or C₁₋₃ alkyl, wherein the C₁₋₃ alkyl is optionally substituted by 1, 2, or 3 Rₑ;
each R₆₁ is independently H, halogen, OH, NH₂, or C₁₋₃ alkyl, wherein the C₁₋₃ alkyl is optionally substituted by 1, 2, or 3 R_{f};
each R₇ is independently H, halogen, OH, NH₂, C₁₋₃ alkyl, —NHC₁₋₃ alkyl, or —N(C₁₋₃ alkyl)₂, wherein the C₁₋₃ alkyl, —NHC₁₋₃ alkyl, and —N(C₁₋₃ alkyl)₂ are each independently and optionally substituted by 1, 2, or 3 R_{g};
or, when E₁ is —C(R₅)₂—, E₂ is —C(R₆₁)₂—, and E₃ is a single bond, R₅ and R₆₁, together with the C atom to which they are attached, make the structural moiety 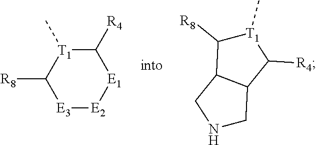
or, when E₂ is —C(R₆₁)₂—, two R₆₁, together with the C atom to which they are attached, make the structural moiety 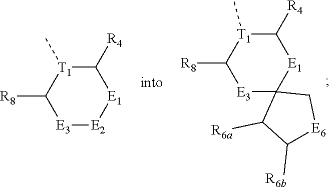
or, when E₃ is a single bond, R₄ and R₈, together with the C atom to which they are attached, make the structural moiety 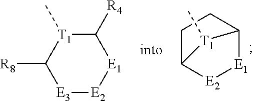
or, when T₁ is CH and E₁ is —C(R₅)₂—, R₅ and R₈, together with the C atom to which they are attached, make the structural moiety 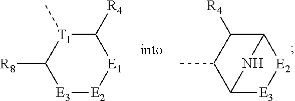
E₆ is NR_{6c} or O;
R₆ₐ and R_{6b} are each independently H, halogen, OH, ═O, NH₂, or C₁₋₃ alkyl;
R_{6c} is H or C₁₋₃ alkyl;
Rₐ, R_{b}, R_{c}, R_{d}, Rₑ, R_{f}, and R_{g} are each independently F, Cl, Br, OH, CN, COOH, NH₂, C₁₋₃ alkyl, or C₁₋₃ alkoxy;
each Rₜ is independently H, F, Cl, Br, OH, CN, COOH, NH₂, C₁₋₃ alkyl or C₁₋₃ alkoxy;
n is 1 or 2;
the 4- to 6-membered heterocycloalkyl contains 1, 2, 3, or 4 heteroatoms or heteroatom groups independently selected from —O—, —NH—, —S—, and N.

2. The compound or the pharmaceutically acceptable salt thereof according to claim 1, wherein the compound is 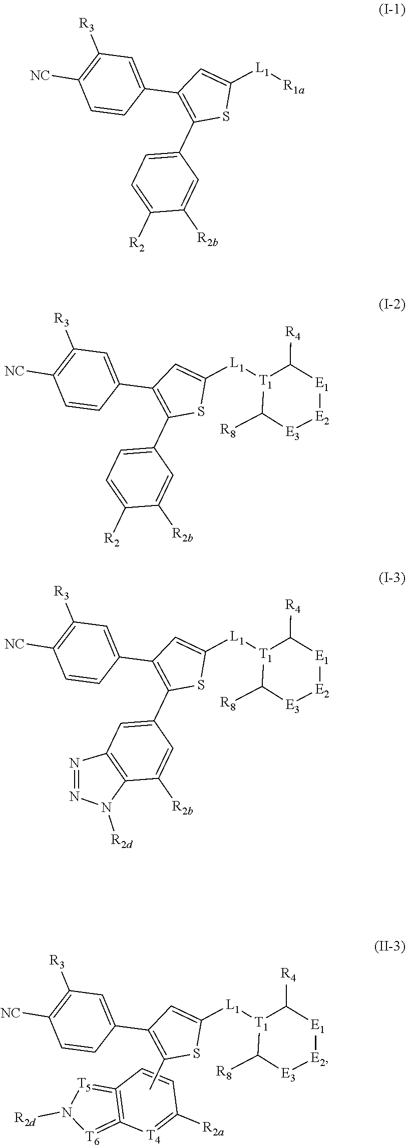
wherein
R₂ is H, halogen, NH₂, C₁₋₃ alkyl, C₁₋₃ alkoxy, —S(═O)₂C₁₋₃ alkyl, —NHC₁₋₃ alkyl, 4- to 6-membered heterocycloalkyl, —C(═O)-4- to 6-membered heterocycloalkyl, or —NHC₁₋₃ alkyl-C₃₋₆ cycloalkyl, wherein the C₁₋₃ alkyl, C₁₋₃ alkoxy, —S(═O)₂C₁₋₃ alkyl, —NHC₁₋₃ alkyl, 4- to 6-membered heterocycloalkyl, —C(═O)-4- to 6-membered heterocycloalkyl, and —NHC₁₋₃ alkyl-C₃₋₆ cycloalkyl are each independently and optionally substituted by 1, 2, or 3 R_{b}.

3. The compound or the pharmaceutically acceptable salt thereof according to claim 1, wherein Rₐ, R_{b}, R_{c}, R_{d}, Rₑ, R_{f}, and R_{g} are each independently F, Cl, Br, OH, CN, COOH, NH₂, CH₃, or OCH₃.

4. The compound or the pharmaceutically acceptable salt thereof according to claim 1, wherein each Rₜ is independently H, F, Cl, or CH₃;
or, R₂ₐ is H or F;
or, R_{2d} is H, CH₃, 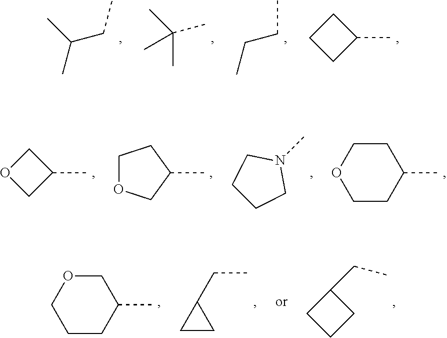
wherein the CH₃, 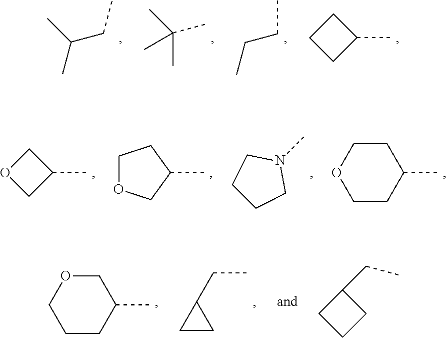
are optionally substituted by 1, 2, or 3 R_{c};
or, R₆ₐ and R_{6b} are each independently H, OH, ═O, NH₂, or CH₃;
or, R_{6c} is H or CH₃.

5. The compound or the pharmaceutically acceptable salt thereof according to claim 1, wherein R₁ₐ is 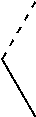
substituted by 1, 2, or 3 Rₐ;
or, R₂ and R_{2b} are each independently H, F, Cl, NH₂, —OCH₃, 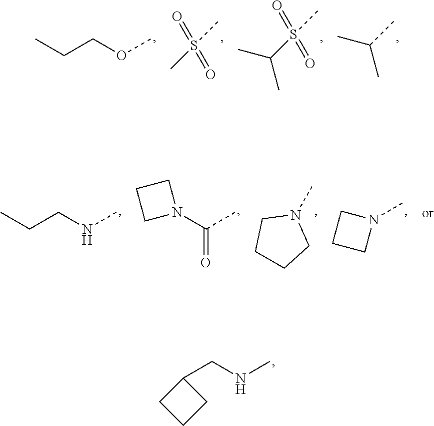
wherein the —OCH₃, 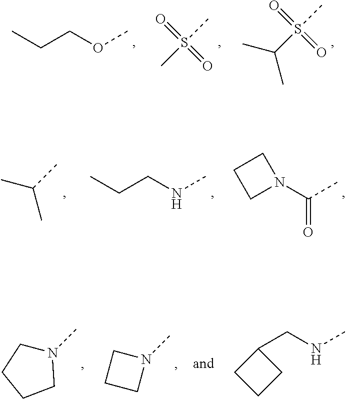
are each independently and optionally substituted by 1, 2, or 3 R_{b},
or, R₂ₑ, R_{2f}, and R_{2g} are each independently H or CH₃;
or, each R₃ is independently F or OCH₃:
or, each R₅ is independently H, F, Cl, Br, I, OH, NH₂, CH₃, —NH—CH₃, or 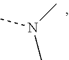
wherein the CH₃, —NH—CH₃, and 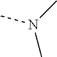
are each independently and optionally substituted by 1, 2, or 3 R_{d};
or, R₆ is H, CH₃, or CH₂—CH₃, wherein the CH₃ and CH₂—CH₃ are each independently and optionally substituted by 1, 2, or 3 Rₑ;
or, each R₆₁ is independently H, F, Cl, Br, I, OH, NH₂, or CH₃, and the CH₃ is optionally substituted by 1, 2, or 3 R_{f};
or, each R₇ is independently H, F, Cl, Br, I, OH, NH₂, CH₃, —NH—CH₃, or 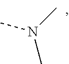
and the CH₃, —NH—CH₃, and 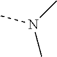
are optionally substituted by 1, 2, or 3 R_{g};
or, the salt is hydrochloride.

6. The compound or the pharmaceutically acceptable salt thereof according to claim 5, wherein R₁ₐ is 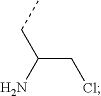
or, R₂ and R_{2b} are each independently H, F, Cl, NH₂, —OCH₃, 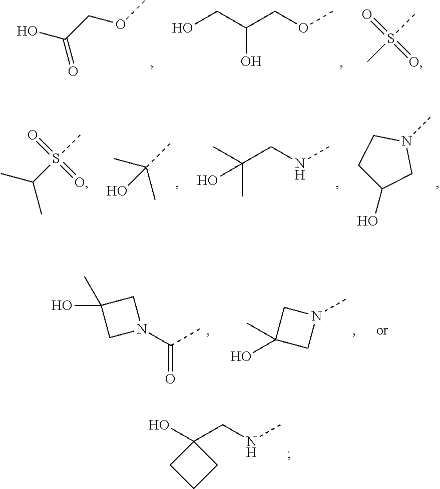
or, each R₅ is independently H, OH, NH₂, CH₃, or 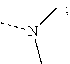
or, R₆ is H, CH₂—CN, or CH₂—CF₃;
or, each R₆₁ is independently H, F, OH, or NH₂;
or, each R₇ is independently H, OH, NH₂, CH₃, or

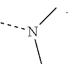

7-10. (canceled)

11. The compound or the pharmaceutically acceptable salt thereof according to claim 1, wherein R_{2d} is H, CH₃,

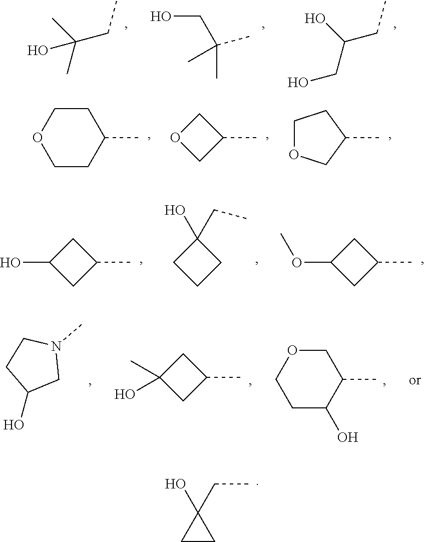

12-13. (canceled)

14. The compound or the pharmaceutically acceptable salt thereof according to claim 1, wherein R₄ and R₈ are each independently H or CH₃.

15-25. (canceled)

26. The compound or the pharmaceutically acceptable salt thereof according to claim 1, wherein the structural moiety

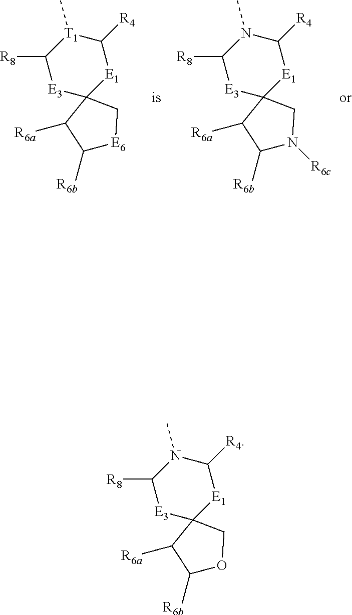

27. The compound or the pharmaceutically acceptable salt thereof according to claim 26, wherein the structural moiety

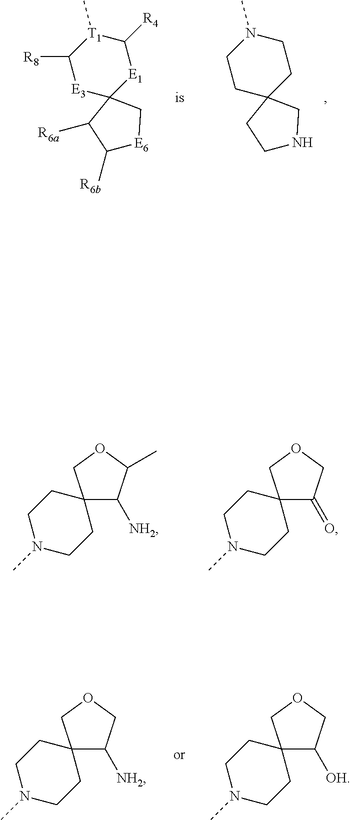

28-29. (canceled)

30. The compound or the pharmaceutically acceptable salt thereof according to claim 1, wherein the structural moiety

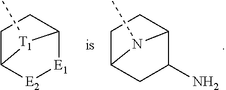

31. The compound or the pharmaceutically acceptable salt thereof according to claim 1, wherein the structural moiety

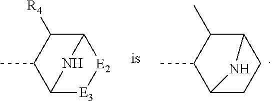

32. The compound or the pharmaceutically acceptable salt thereof according to claim 1, wherein R₁ is 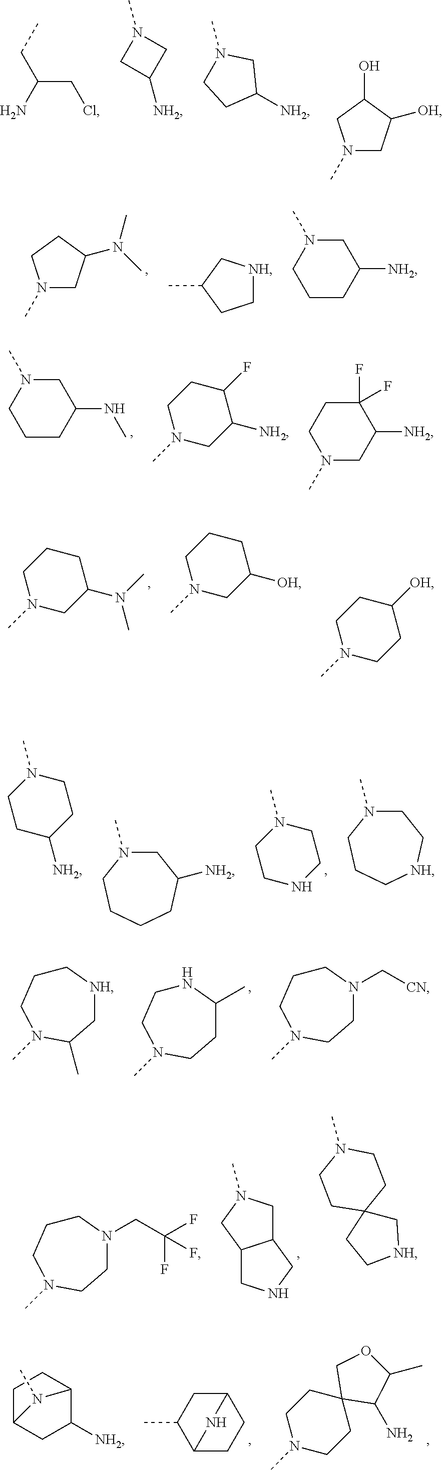 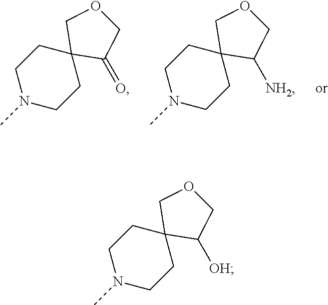
or, ring A is

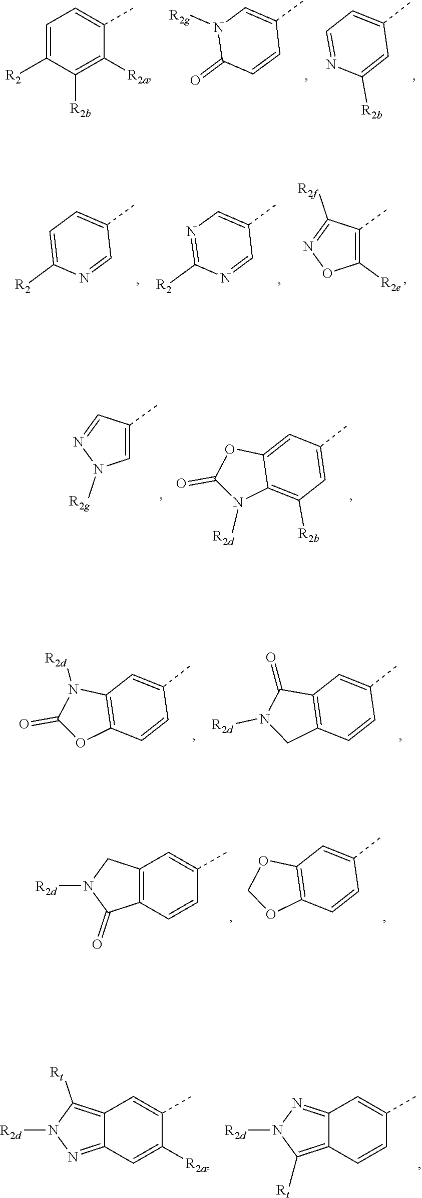

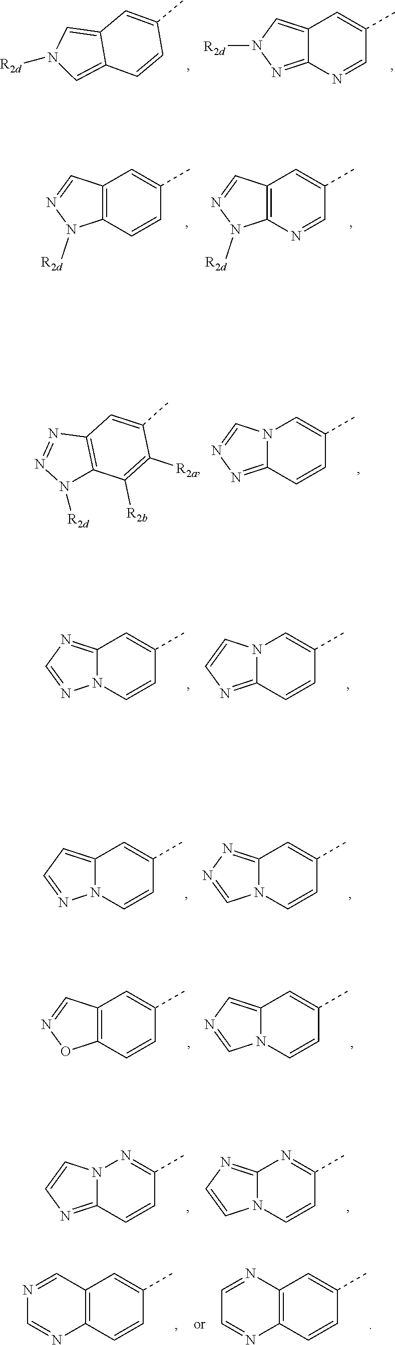

33. (canceled)

34. The compound or the pharmaceutically acceptable salt thereof according to claim 32, wherein ring A is

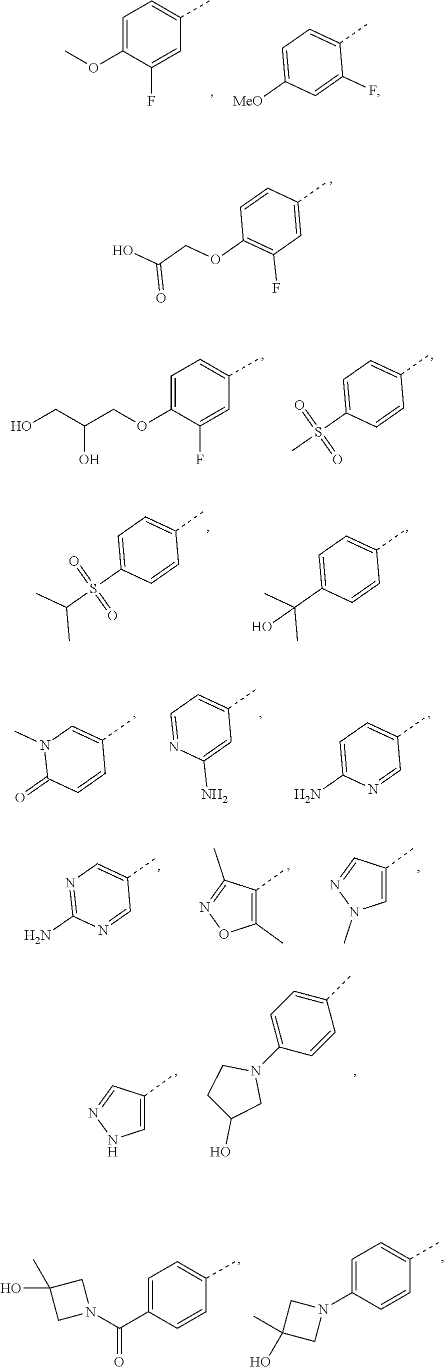

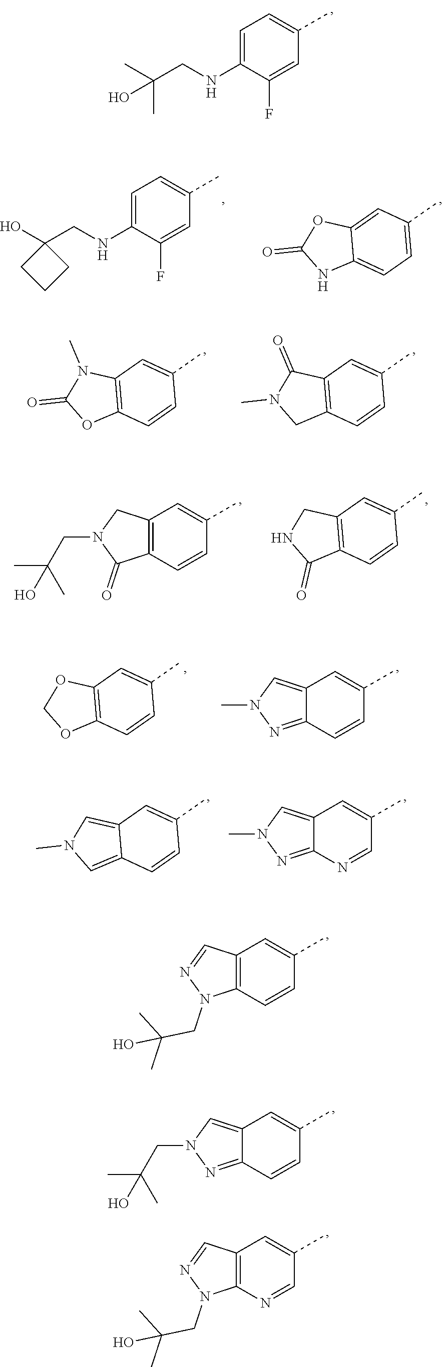

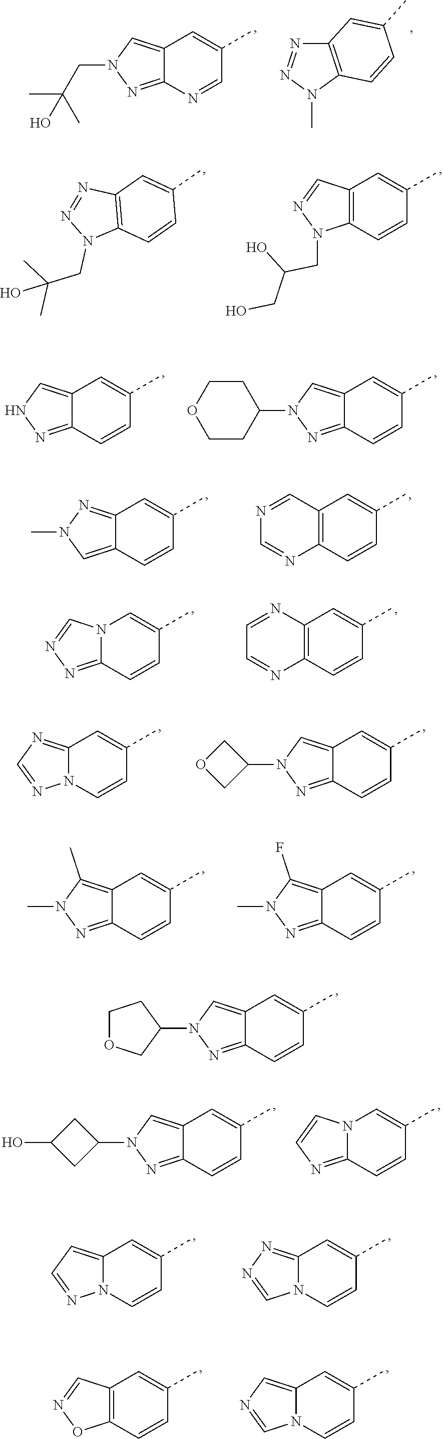

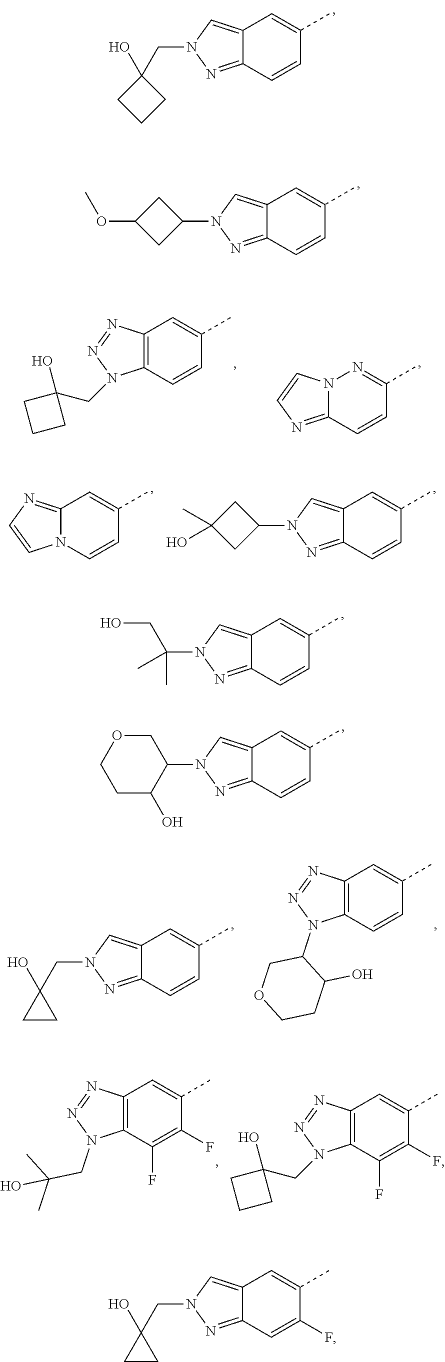

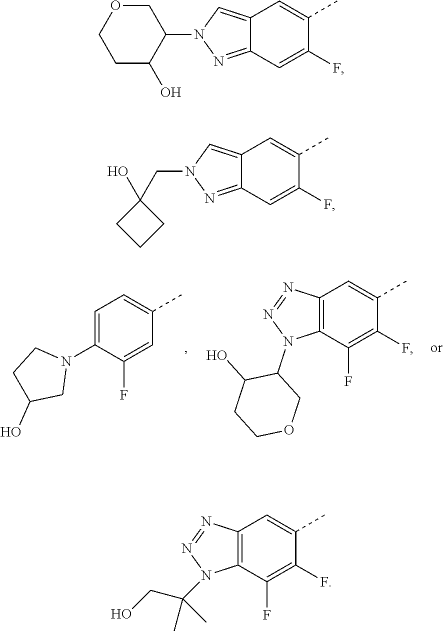

35. The compound or the pharmaceutically acceptable salt thereof according to claim 1, wherein the compound is 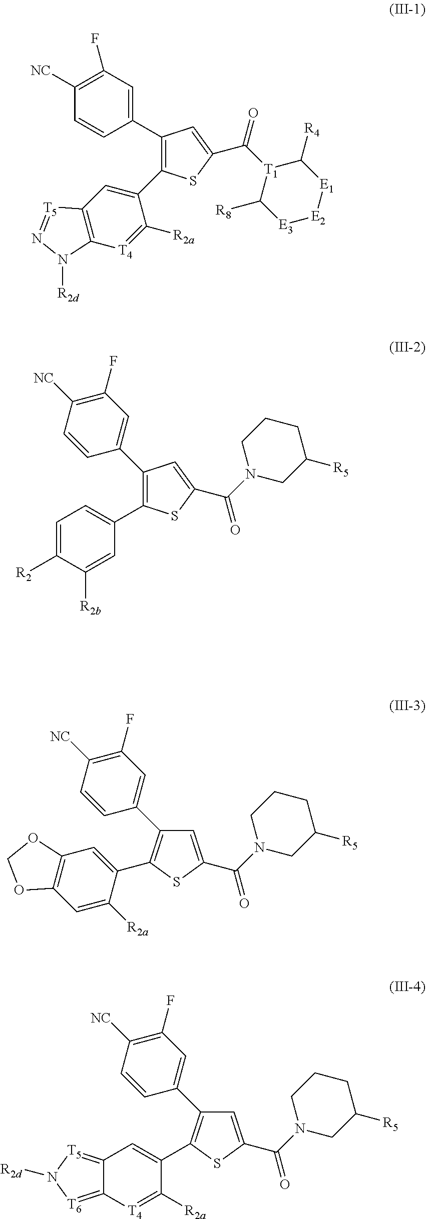
wherein
R₂ and R_{2b} are each independently H, halogen, NH₂, C₁₋₃ alkyl, C₁₋₃ alkoxy, —S(═O)₂C₁₋₃ alkyl, 4- to 6-membered heterocycloalkyl, or —C(═O)-4- to 6-membered heterocycloalkyl, wherein the C₁₋₃ alkyl, C₁₋₃ alkoxy, —S(═O)₂C₁₋₃ alkyl, 4- to 6-membered heterocycloalkyl, and —C(═O)-4- to 6-membered heterocycloalkyl are each independently and optionally substituted by 1, 2, or 3 R_{b}.

36. The compound or the pharmaceutically acceptable salt thereof according to claim 35, wherein the compound is

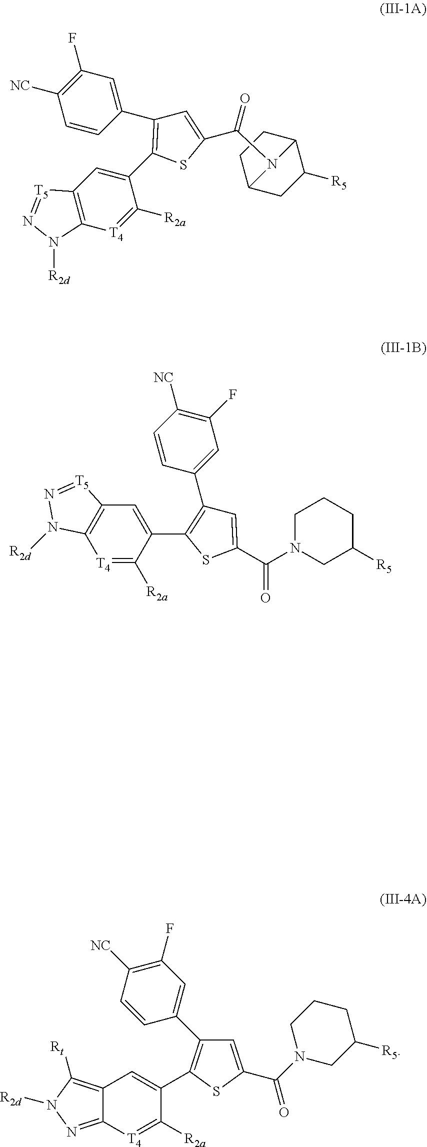

37. The compound or the pharmaceutically acceptable salt thereof according to claim 36, wherein the compound is

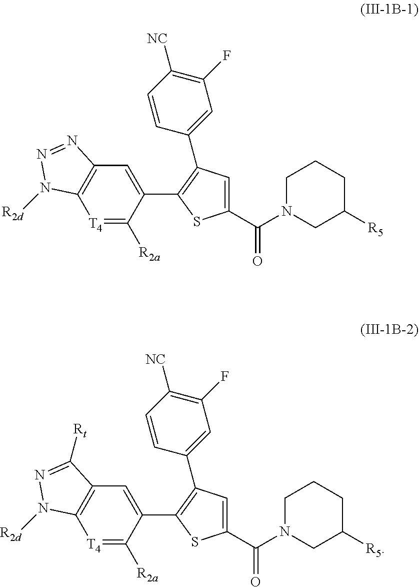

38. A compound of the following formula or a pharmaceutically acceptable salt thereof, selected from,

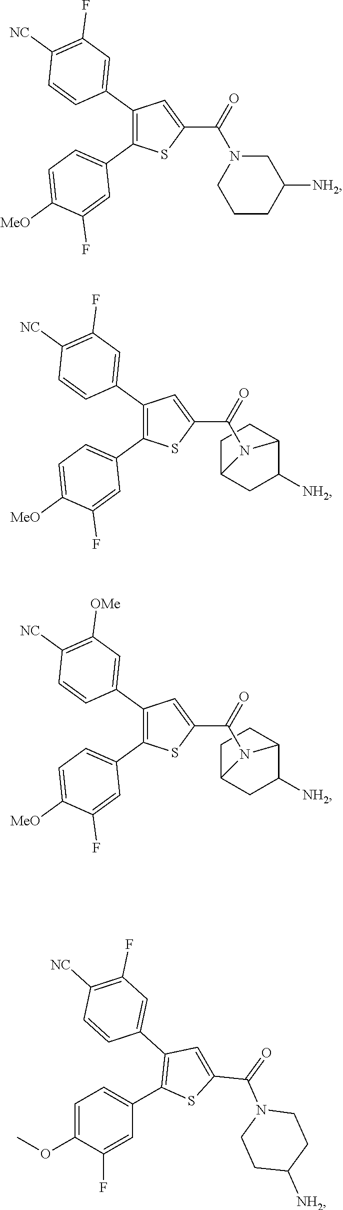 [SMILES: COc1ccc(-c2sc(C(=O)N3CCCC(N)C3)cc2-c2ccc(C#N)c(F)c2)cc1F ; COc1ccc(-c2sc(C(=O)N3C4CCC3C(N)C4)cc2-c2ccc(C#N)c(F)c2)cc1F ; COc1ccc(-c2sc(C(=O)N3C4CCC3C(N)C4)cc2-c2ccc(C#N)c(OC)c2)cc1F ; COc1ccc(-c2sc(C(=O)N3CCC(N)CC3)cc2-c2ccc(C#N)c(F)c2)cc1F]

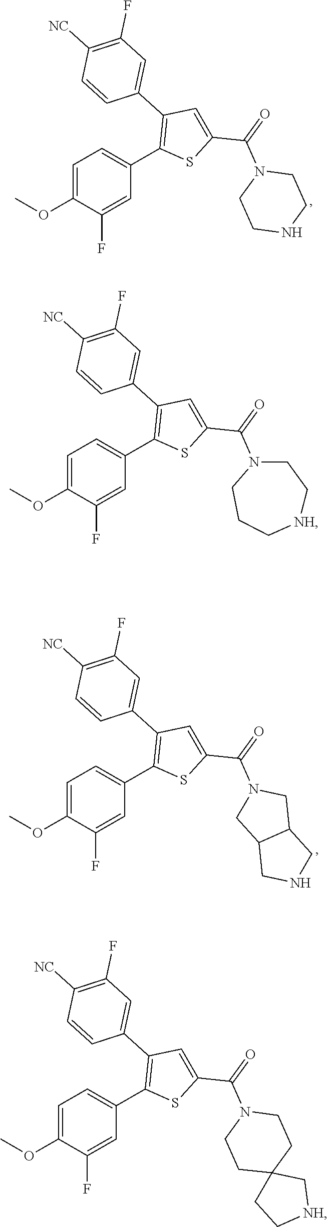 [SMILES: COc1ccc(-c2sc(C(=O)N3CCNCC3)cc2-c2ccc(C#N)c(F)c2)cc1F ; COc1ccc(-c2sc(C(=O)N3CCCNCC3)cc2-c2ccc(C#N)c(F)c2)cc1F ; COc1ccc(-c2sc(C(=O)N3CC4CNCC4C3)cc2-c2ccc(C#N)c(F)c2)cc1F ; COc1ccc(-c2sc(C(=O)N3CCC4(CCNC4)CC3)cc2-c2ccc(C#N)c(F)c2)cc1F]

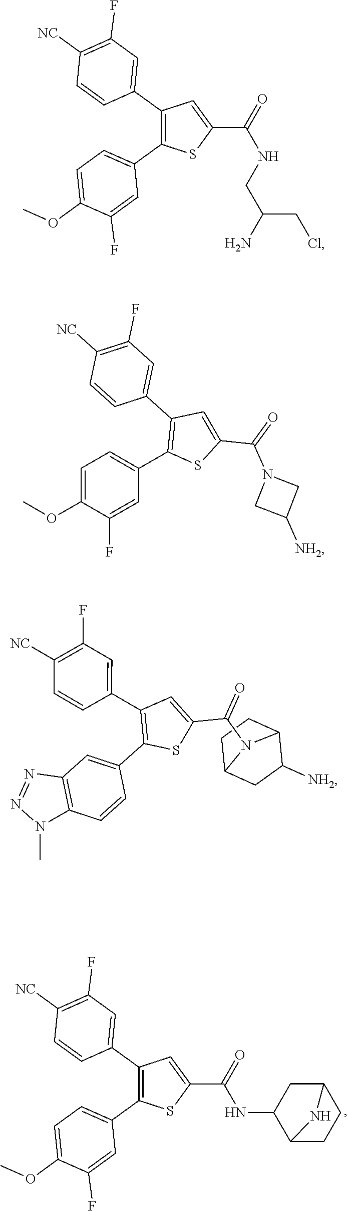 [SMILES: COc1ccc(-c2sc(C(=O)NCC(N)CCl)cc2-c2ccc(C#N)c(F)c2)cc1F ; COc1ccc(-c2sc(C(=O)N3CC(N)C3)cc2-c2ccc(C#N)c(F)c2)cc1F ; Cn1nnc2cc(-c3sc(C(=O)N4C5CCC4C(N)C5)cc3-c3ccc(C#N)c(F)c3)ccc21 ; COc1ccc(-c2sc(C(=O)NC3CC4CCC3N4)cc2-c2ccc(C#N)c(F)c2)cc1F]

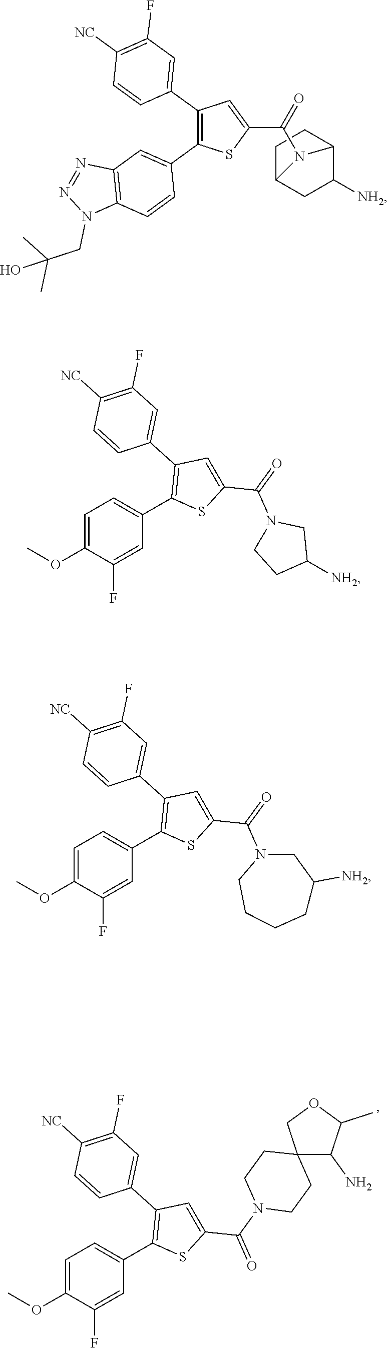

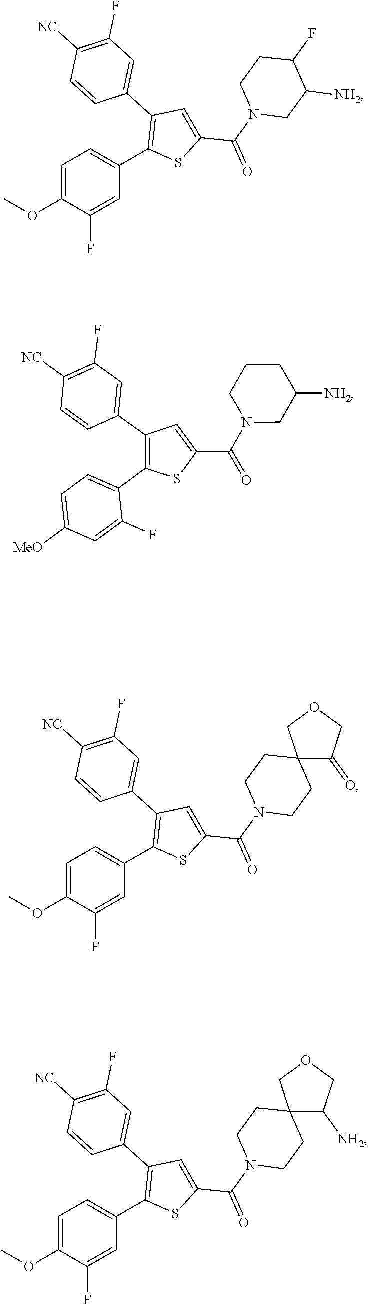 [SMILES: COc1ccc(-c2sc(C(=O)N3CCC(F)C(N)C3)cc2-c2ccc(C#N)c(F)c2)cc1F ; COc1ccc(-c2sc(C(=O)N3CCCC(N)C3)cc2-c2ccc(C#N)c(F)c2)c(F)c1 ; COc1ccc(-c2sc(C(=O)N3CCC4(CC3)COCC4=O)cc2-c2ccc(C#N)c(F)c2)cc1F ; COc1ccc(-c2sc(C(=O)N3CCC4(CC3)COCC4N)cc2-c2ccc(C#N)c(F)c2)cc1F]

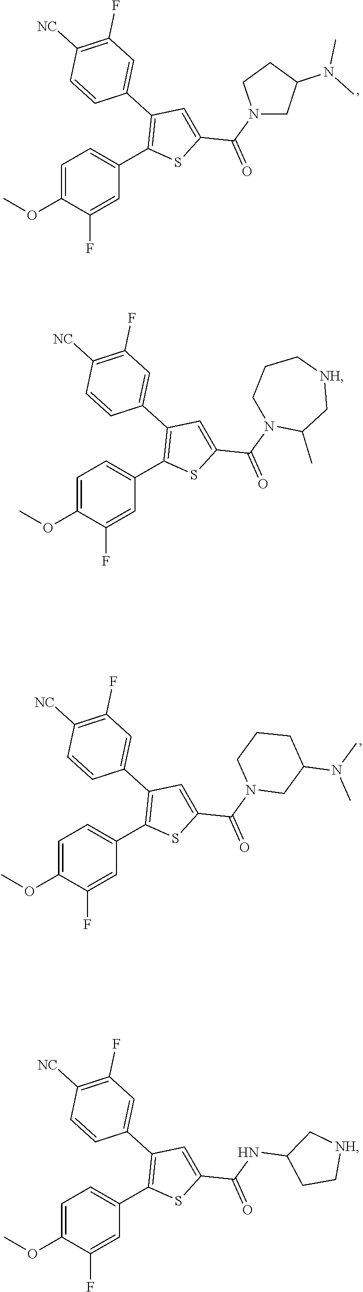

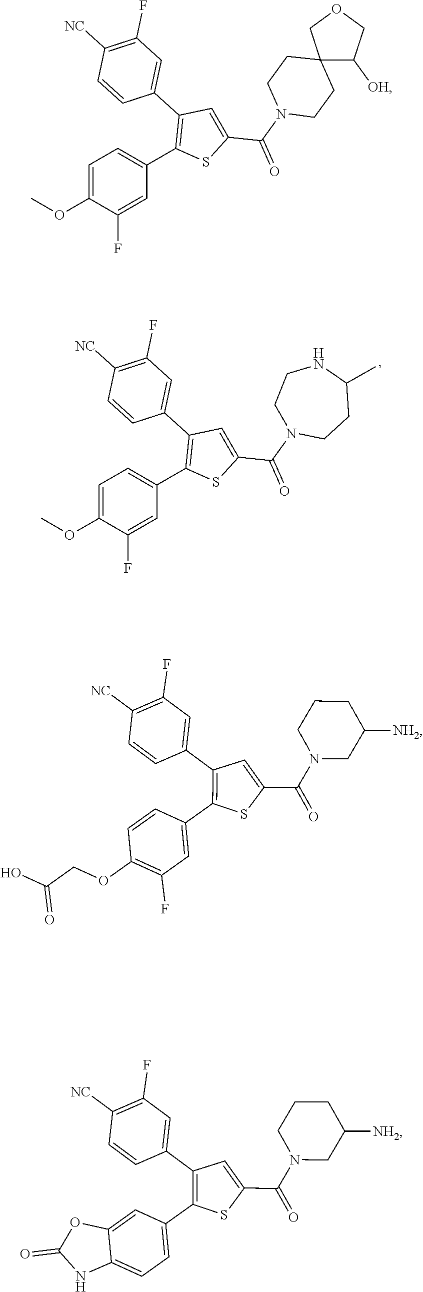

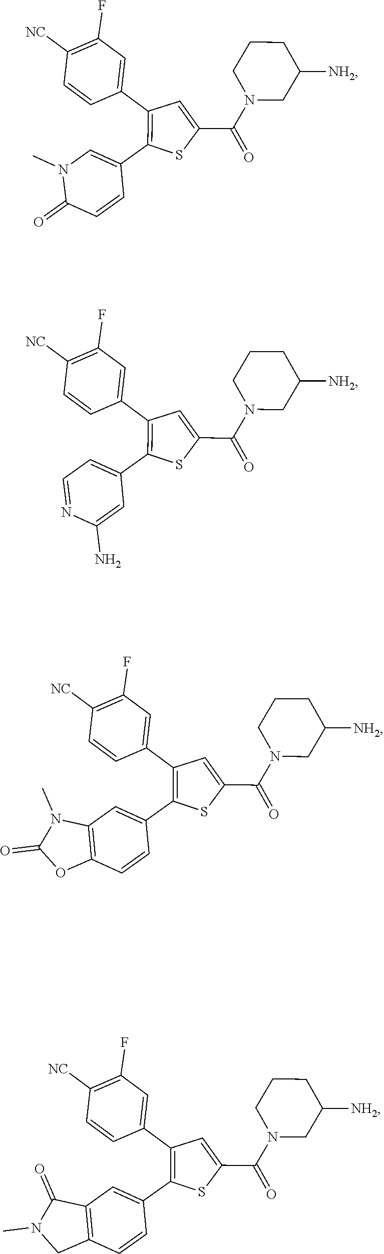 [SMILES: Cn1cc(-c2sc(C(=O)N3CCCC(N)C3)cc2-c2ccc(C#N)c(F)c2)ccc1=O ; N#Cc1ccc(-c2cc(C(=O)N3CCCC(N)C3)sc2-c2ccnc(N)c2)cc1F ; Cn1c(=O)oc2ccc(-c3sc(C(=O)N4CCCC(N)C4)cc3-c3ccc(C#N)c(F)c3)cc21 ; CN1Cc2ccc(-c3sc(C(=O)N4CCCC(N)C4)cc3-c3ccc(C#N)c(F)c3)cc2C1=O]

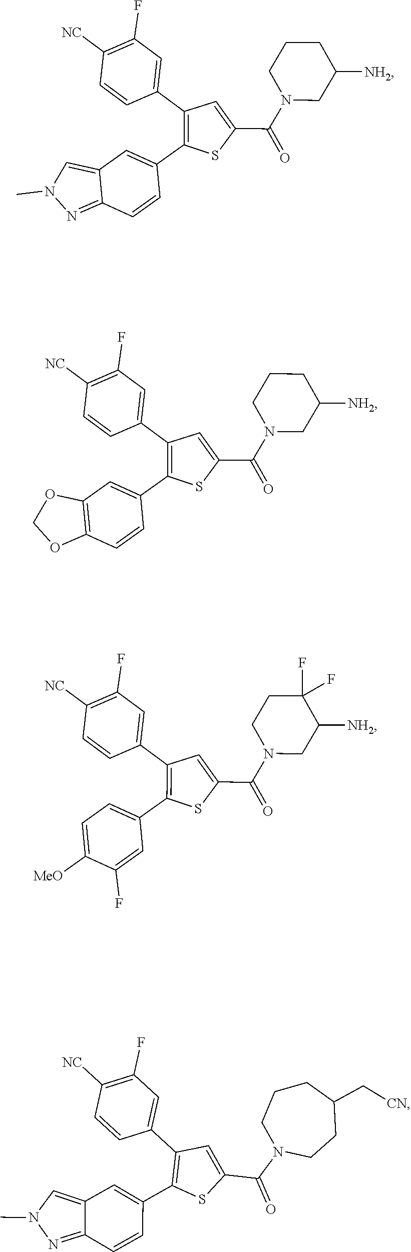 [SMILES: Cn1cc2cc(-c3sc(C(=O)N4CCCC(N)C4)cc3-c3ccc(C#N)c(F)c3)ccc2n1 ; N#Cc1ccc(-c2cc(C(=O)N3CCCC(N)C3)sc2-c2ccc3c(c2)OCO3)cc1F ; COc1ccc(-c2sc(C(=O)N3CCC(F)(F)C(N)C3)cc2-c2ccc(C#N)c(F)c2)cc1F ; Cn1cc2cc(-c3sc(C(=O)N4CCCC(CC#N)CC4)cc3-c3ccc(C#N)c(F)c3)ccc2n1]

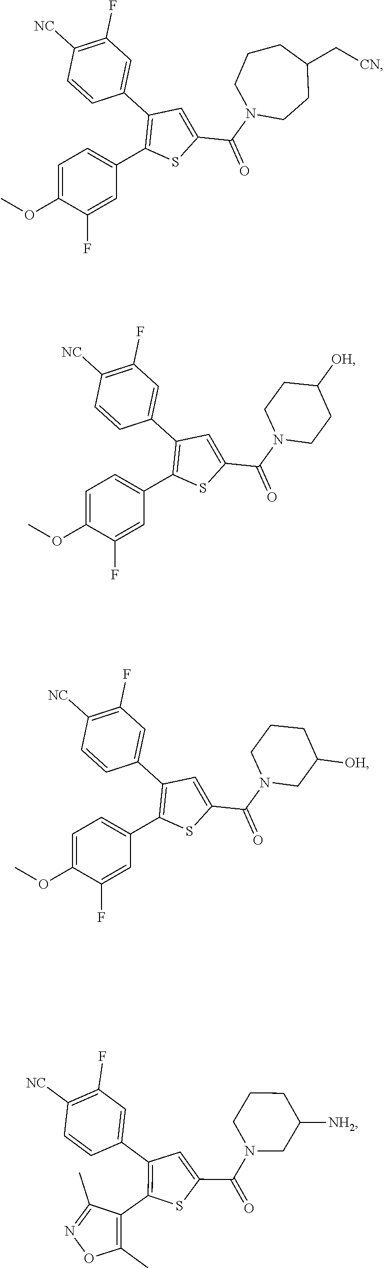 [SMILES: COc1ccc(-c2sc(C(=O)N3CCCC(CC#N)CC3)cc2-c2ccc(C#N)c(F)c2)cc1F ; COc1ccc(-c2sc(C(=O)N3CCC(O)CC3)cc2-c2ccc(C#N)c(F)c2)cc1F ; COc1ccc(-c2sc(C(=O)N3CCCC(O)C3)cc2-c2ccc(C#N)c(F)c2)cc1F ; Cc1noc(C)c1-c1sc(C(=O)N2CCCC(N)C2)cc1-c1ccc(C#N)c(F)c1]

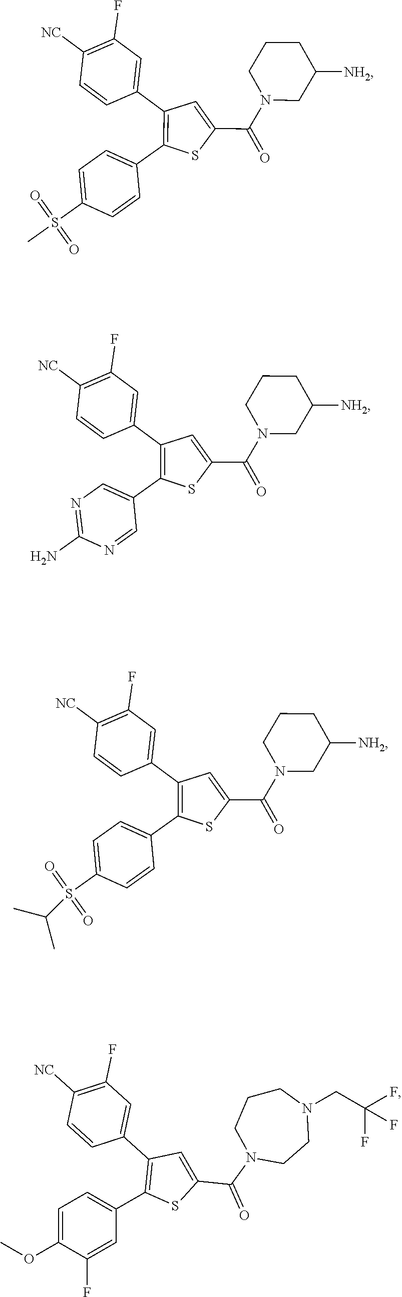 [SMILES: CS(=O)(=O)c1ccc(-c2sc(C(=O)N3CCCC(N)C3)cc2-c2ccc(C#N)c(F)c2)cc1 ; N#Cc1ccc(-c2cc(C(=O)N3CCCC(N)C3)sc2-c2cnc(N)nc2)cc1F ; CC(C)S(=O)(=O)c1ccc(-c2sc(C(=O)N3CCCC(N)C3)cc2-c2ccc(C#N)c(F)c2)cc1 ; COc1ccc(-c2sc(C(=O)N3CCCN(CC(F)(F)F)CC3)cc2-c2ccc(C#N)c(F)c2)cc1F]

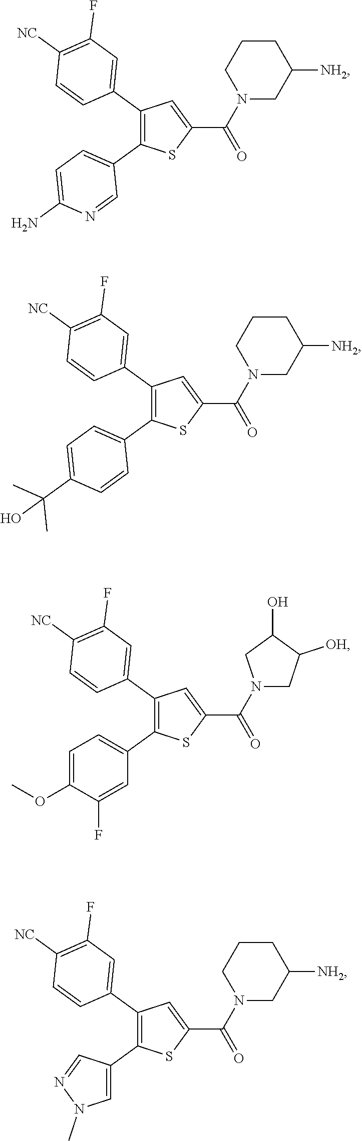 [SMILES: N#Cc1ccc(-c2cc(C(=O)N3CCCC(N)C3)sc2-c2ccc(N)nc2)cc1F ; CC(C)(O)c1ccc(-c2sc(C(=O)N3CCCC(N)C3)cc2-c2ccc(C#N)c(F)c2)cc1 ; COc1ccc(-c2sc(C(=O)N3CC(O)C(O)C3)cc2-c2ccc(C#N)c(F)c2)cc1F ; Cn1cc(-c2sc(C(=O)N3CCCC(N)C3)cc2-c2ccc(C#N)c(F)c2)cn1]

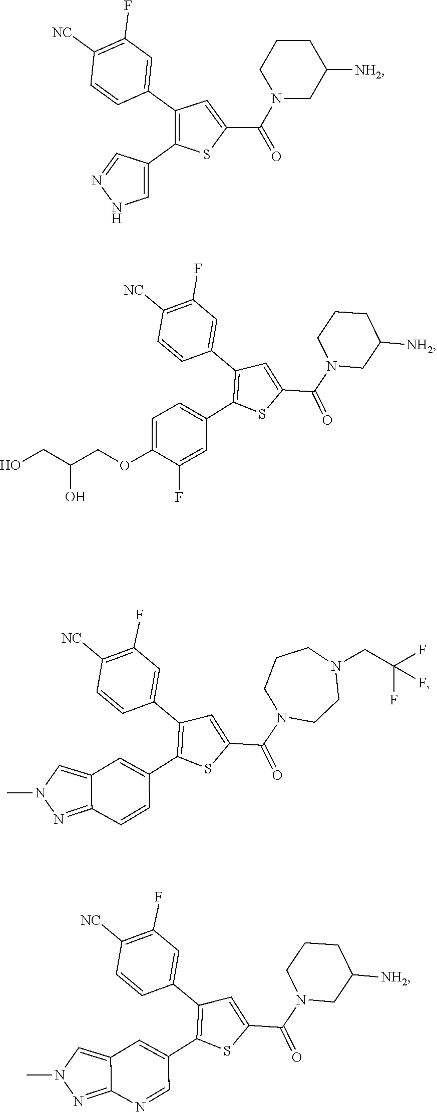 [SMILES: N#Cc1ccc(-c2cc(C(=O)N3CCCC(N)C3)sc2-c2cn[nH]c2)cc1F ; N#Cc1ccc(-c2cc(C(=O)N3CCCC(N)C3)sc2-c2ccc(OCC(O)CO)c(F)c2)cc1F ; Cn1cc2cc(-c3sc(C(=O)N4CCCN(CC(F)(F)F)CC4)cc3-c3ccc(C#N)c(F)c3)ccc2n1 ; Cn1cc2cc(-c3sc(C(=O)N4CCCC(N)C4)cc3-c3ccc(C#N)c(F)c3)cnc2n1]

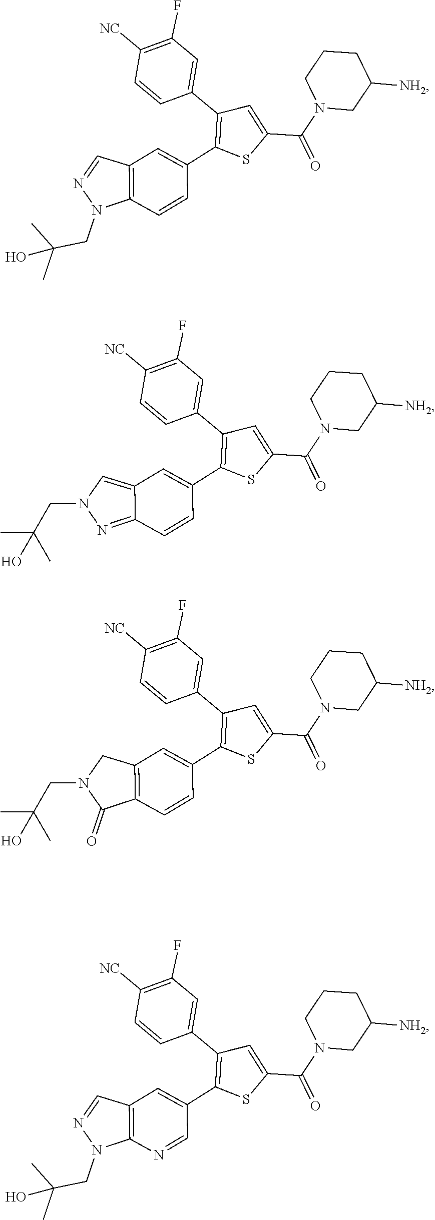 [SMILES: CC(C)(O)Cn1ncc2cc(-c3sc(C(=O)N4CCCC(N)C4)cc3-c3ccc(C#N)c(F)c3)ccc21 ; CC(C)(O)Cn1cc2cc(-c3sc(C(=O)N4CCCC(N)C4)cc3-c3ccc(C#N)c(F)c3)ccc2n1 ; CC(C)(O)CN1Cc2cc(-c3sc(C(=O)N4CCCC(N)C4)cc3-c3ccc(C#N)c(F)c3)ccc2C1=O ; CC(C)(O)Cn1ncc2cc(-c3sc(C(=O)N4CCCC(N)C4)cc3-c3ccc(C#N)c(F)c3)cnc21]

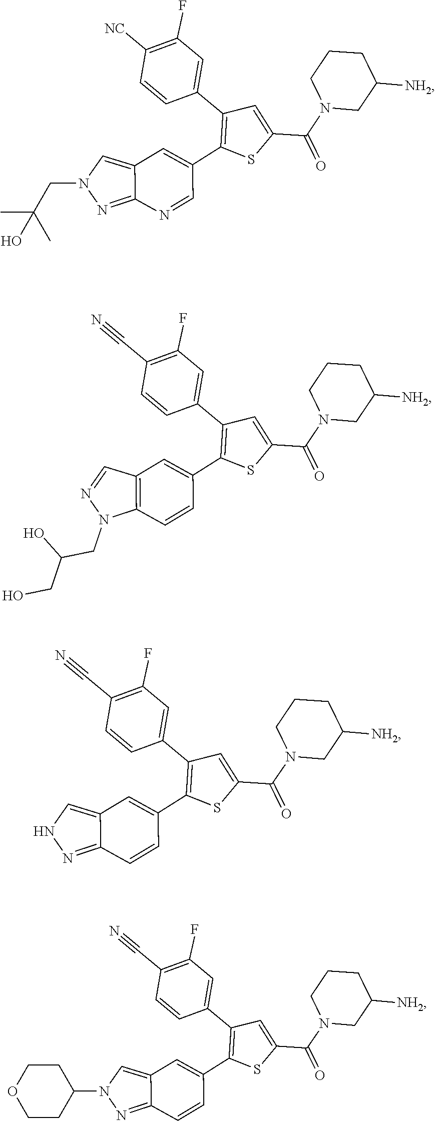 [SMILES: CC(C)(O)Cn1cc2cc(-c3sc(C(=O)N4CCCC(N)C4)cc3-c3ccc(C#N)c(F)c3)cnc2n1 ; N#Cc1ccc(-c2cc(C(=O)N3CCCC(N)C3)sc2-c2ccc3c(cnn3CC(O)CO)c2)cc1F ; N#Cc1ccc(-c2cc(C(=O)N3CCCC(N)C3)sc2-c2ccc3n[nH]cc3c2)cc1F ; N#Cc1ccc(-c2cc(C(=O)N3CCCC(N)C3)sc2-c2ccc3nn(C4CCOCC4)cc3c2)cc1F]

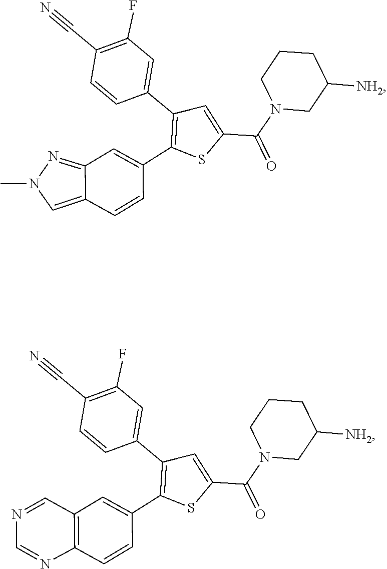 [SMILES: Cn1cc2ccc(-c3sc(C(=O)N4CCCC(N)C4)cc3-c3ccc(C#N)c(F)c3)cc2n1 ; N#Cc1ccc(-c2cc(C(=O)N3CCCC(N)C3)sc2-c2ccc3ncncc3c2)cc1F]

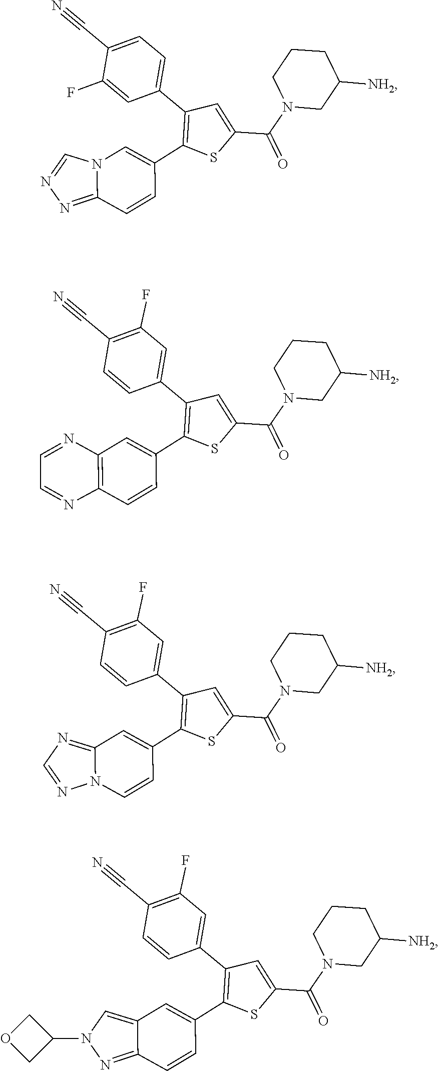 [SMILES: N#Cc1ccc(-c2cc(C(=O)N3CCCC(N)C3)sc2-c2ccc3nncn3c2)cc1F ; N#Cc1ccc(-c2cc(C(=O)N3CCCC(N)C3)sc2-c2ccc3nccnc3c2)cc1F ; N#Cc1ccc(-c2cc(C(=O)N3CCCC(N)C3)sc2-c2ccn3ncnc3c2)cc1F ; N#Cc1ccc(-c2cc(C(=O)N3CCCC(N)C3)sc2-c2ccc3nn(C4COC4)cc3c2)cc1F]

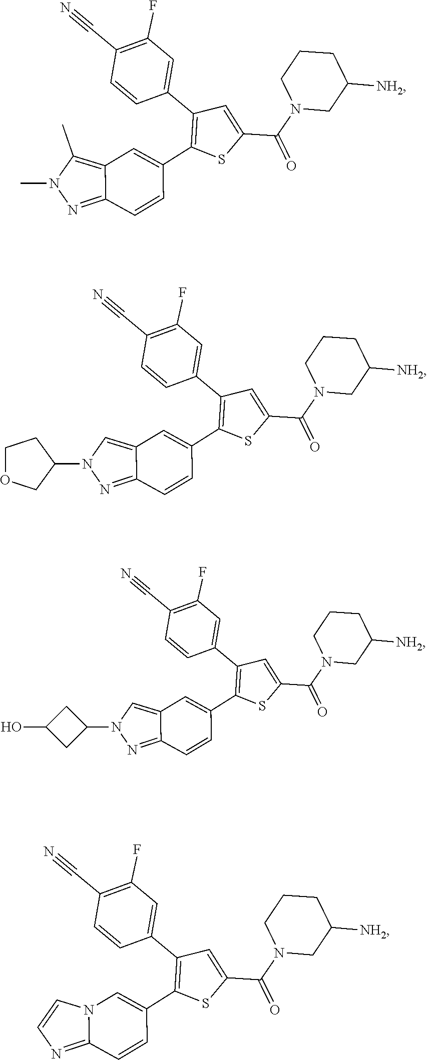 [SMILES: Cc1c2cc(-c3sc(C(=O)N4CCCC(N)C4)cc3-c3ccc(C#N)c(F)c3)ccc2nn1C ; N#Cc1ccc(-c2cc(C(=O)N3CCCC(N)C3)sc2-c2ccc3nn(C4CCOC4)cc3c2)cc1F ; N#Cc1ccc(-c2cc(C(=O)N3CCCC(N)C3)sc2-c2ccc3nn(C4CC(O)C4)cc3c2)cc1F ; N#Cc1ccc(-c2cc(C(=O)N3CCCC(N)C3)sc2-c2ccc3nccn3c2)cc1F]

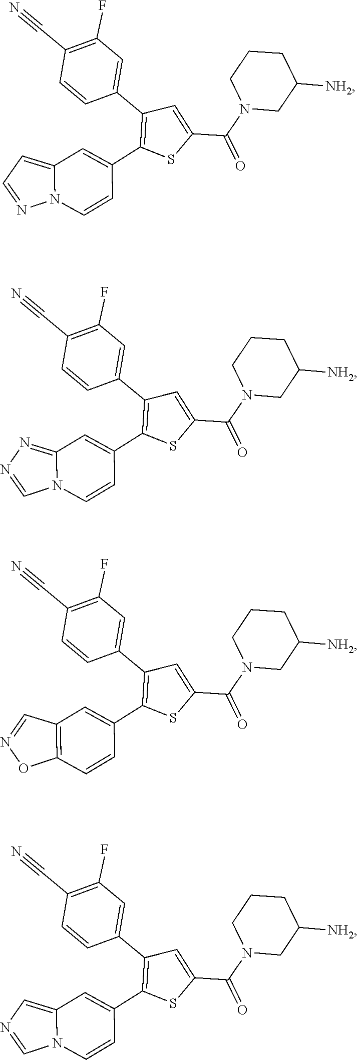 [SMILES: N#Cc1ccc(-c2cc(C(=O)N3CCCC(N)C3)sc2-c2ccn3nccc3c2)cc1F ; N#Cc1ccc(-c2cc(C(=O)N3CCCC(N)C3)sc2-c2ccn3cnnc3c2)cc1F ; N#Cc1ccc(-c2cc(C(=O)N3CCCC(N)C3)sc2-c2ccc3oncc3c2)cc1F ; N#Cc1ccc(-c2cc(C(=O)N3CCCC(N)C3)sc2-c2ccn3cncc3c2)cc1F]

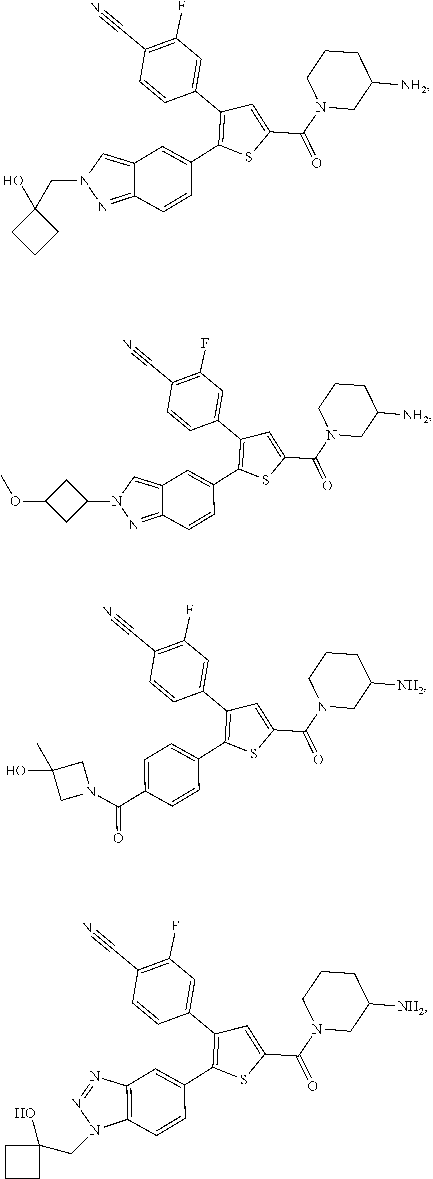 [SMILES: N#Cc1ccc(-c2cc(C(=O)N3CCCC(N)C3)sc2-c2ccc3nn(CC4(O)CCC4)cc3c2)cc1F ; COC1CC(n2cc3cc(-c4sc(C(=O)N5CCCC(N)C5)cc4-c4ccc(C#N)c(F)c4)ccc3n2)C1 ; CC1(O)CN(C(=O)c2ccc(-c3sc(C(=O)N4CCCC(N)C4)cc3-c3ccc(C#N)c(F)c3)cc2)C1 ; N#Cc1ccc(-c2cc(C(=O)N3CCCC(N)C3)sc2-c2ccc3c(c2)nnn3CC2(O)CCC2)cc1F]

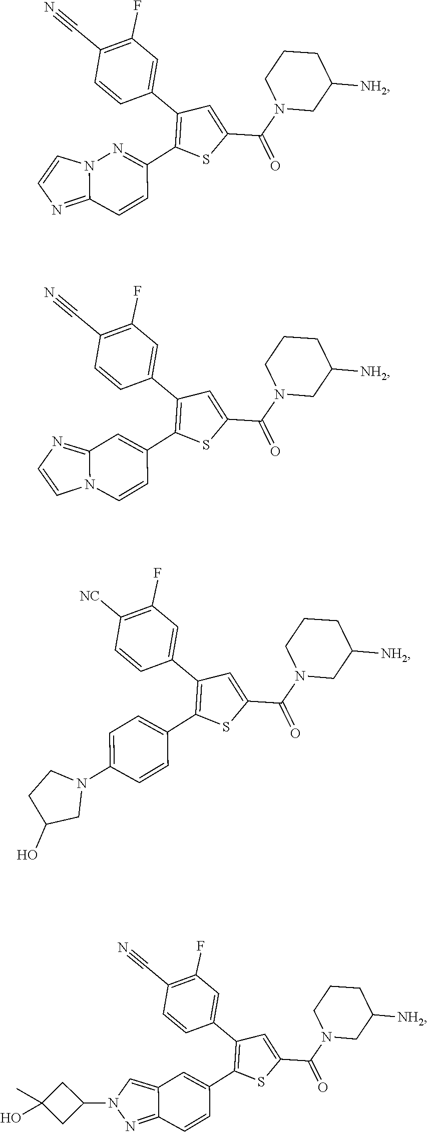 [SMILES: N#Cc1ccc(-c2cc(C(=O)N3CCCC(N)C3)sc2-c2ccc3nccn3n2)cc1F ; N#Cc1ccc(-c2cc(C(=O)N3CCCC(N)C3)sc2-c2ccn3ccnc3c2)cc1F ; N#Cc1ccc(-c2cc(C(=O)N3CCCC(N)C3)sc2-c2ccc(N3CCC(O)C3)cc2)cc1F ; CC1(O)CC(n2cc3cc(-c4sc(C(=O)N5CCCC(N)C5)cc4-c4ccc(C#N)c(F)c4)ccc3n2)C1]

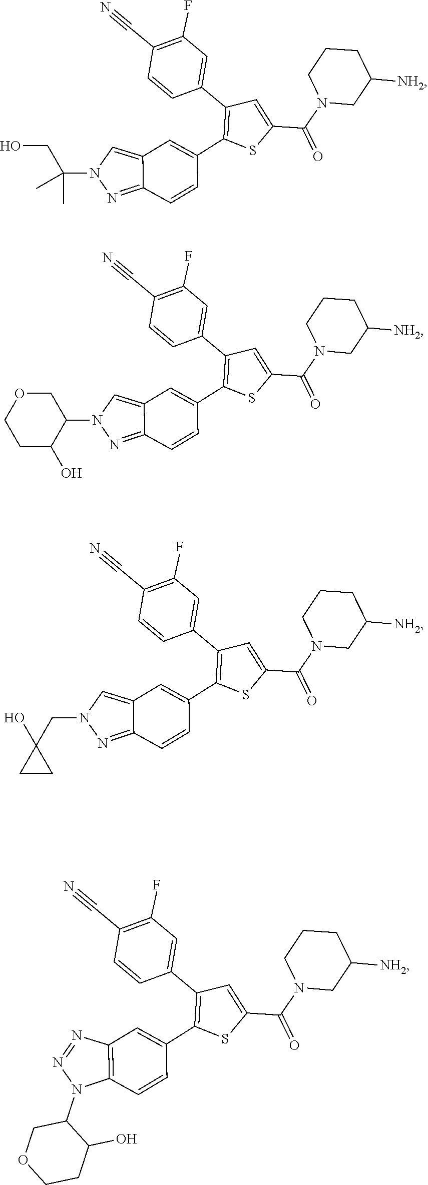 [SMILES: CC(C)(CO)n1cc2cc(-c3sc(C(=O)N4CCCC(N)C4)cc3-c3ccc(C#N)c(F)c3)ccc2n1 ; N#Cc1ccc(-c2cc(C(=O)N3CCCC(N)C3)sc2-c2ccc3nn(C4COCCC4O)cc3c2)cc1F ; N#Cc1ccc(-c2cc(C(=O)N3CCCC(N)C3)sc2-c2ccc3nn(CC4(O)CC4)cc3c2)cc1F ; N#Cc1ccc(-c2cc(C(=O)N3CCCC(N)C3)sc2-c2ccc3c(c2)nnn3C2COCCC2O)cc1F]

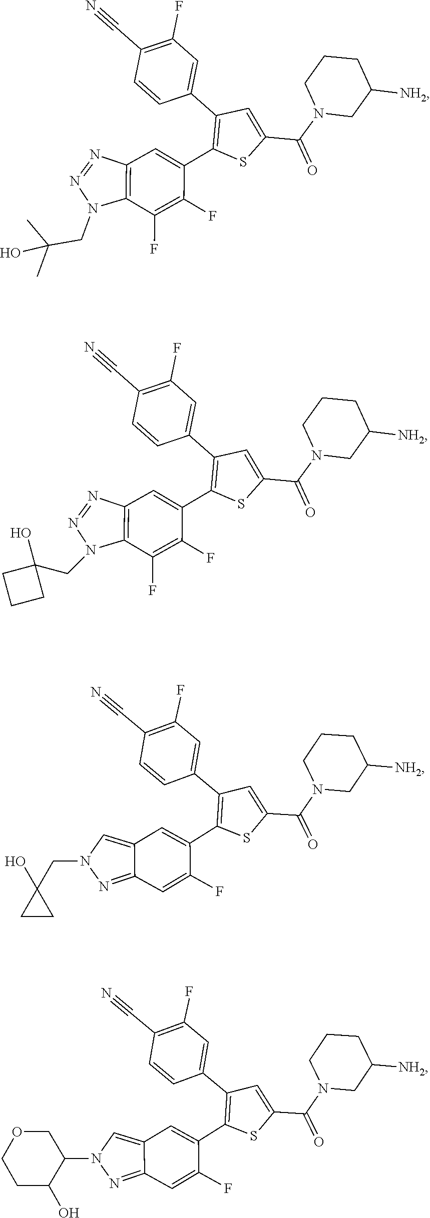 [SMILES: CC(C)(O)Cn1nnc2cc(-c3sc(C(=O)N4CCCC(N)C4)cc3-c3ccc(C#N)c(F)c3)c(F)c(F)c21 ; N#Cc1ccc(-c2cc(C(=O)N3CCCC(N)C3)sc2-c2cc3nnn(CC4(O)CCC4)c3c(F)c2F)cc1F ; N#Cc1ccc(-c2cc(C(=O)N3CCCC(N)C3)sc2-c2cc3cn(CC4(O)CC4)nc3cc2F)cc1F ; N#Cc1ccc(-c2cc(C(=O)N3CCCC(N)C3)sc2-c2cc3cn(C4COCCC4O)nc3cc2F)cc1F]

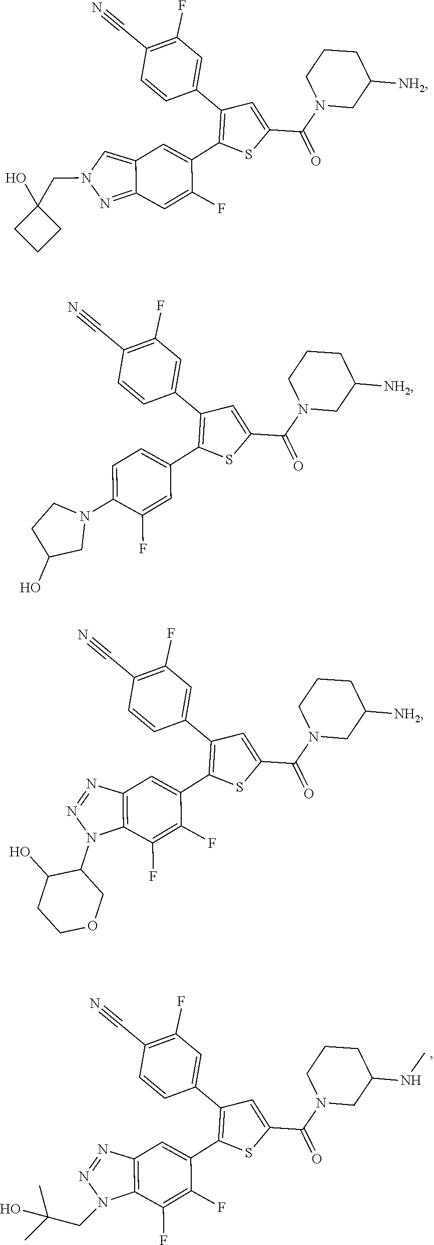 [SMILES: N#Cc1ccc(-c2cc(C(=O)N3CCCC(N)C3)sc2-c2cc3cn(CC4(O)CCC4)nc3cc2F)cc1F ; N#Cc1ccc(-c2cc(C(=O)N3CCCC(N)C3)sc2-c2ccc(N3CCC(O)C3)c(F)c2)cc1F ; N#Cc1ccc(-c2cc(C(=O)N3CCCC(N)C3)sc2-c2cc3nnn(C4COCCC4O)c3c(F)c2F)cc1F ; CNC1CCCN(C(=O)c2cc(-c3ccc(C#N)c(F)c3)c(-c3cc4nnn(CC(C)(C)O)c4c(F)c3F)s2)C1]

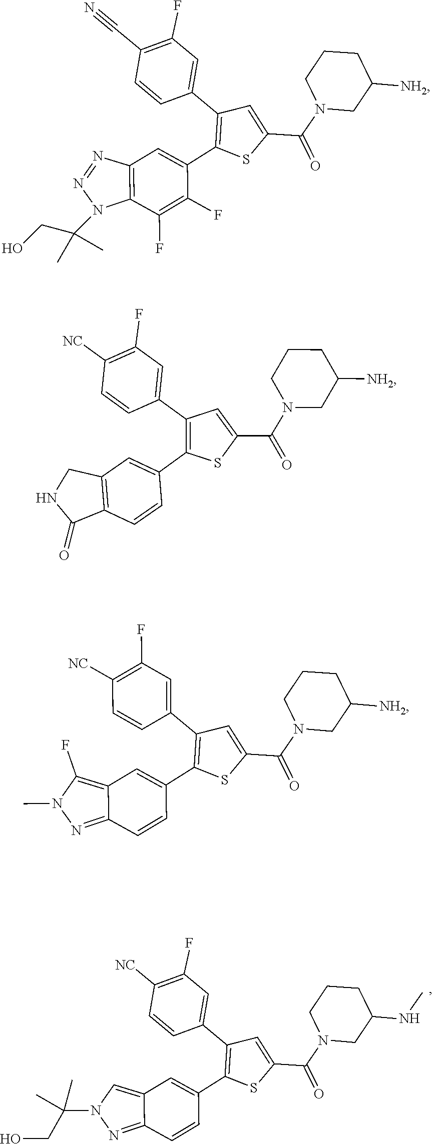 [SMILES: CC(C)(CO)n1nnc2cc(-c3sc(C(=O)N4CCCC(N)C4)cc3-c3ccc(C#N)c(F)c3)c(F)c(F)c21 ; N#Cc1ccc(-c2cc(C(=O)N3CCCC(N)C3)sc2-c2ccc3c(c2)CNC3=O)cc1F ; Cn1nc2ccc(-c3sc(C(=O)N4CCCC(N)C4)cc3-c3ccc(C#N)c(F)c3)cc2c1F ; CNC1CCCN(C(=O)c2cc(-c3ccc(C#N)c(F)c3)c(-c3ccc4nn(C(C)(C)CO)cc4c3)s2)C1]

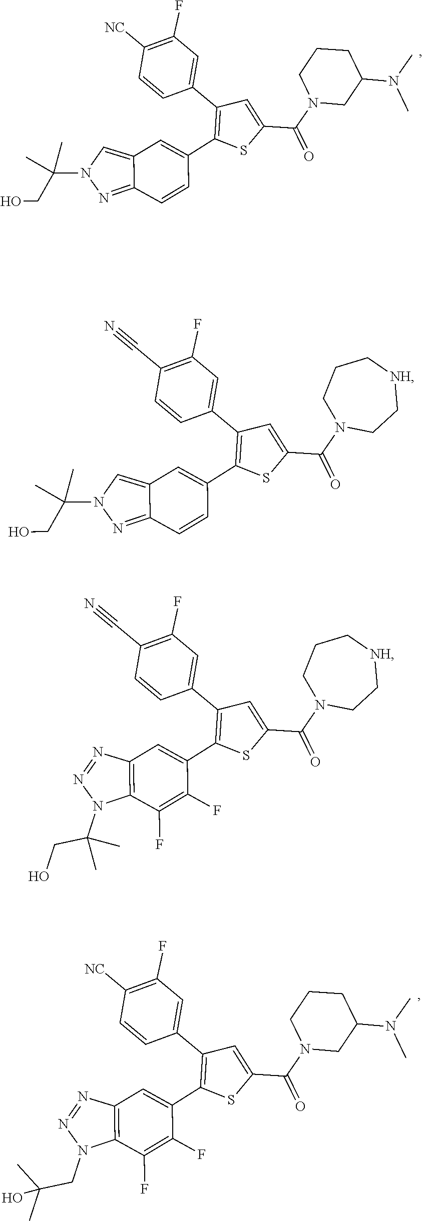 [SMILES: CN(C)C1CCCN(C(=O)c2cc(-c3ccc(C#N)c(F)c3)c(-c3ccc4nn(C(C)(C)CO)cc4c3)s2)C1 ; CC(C)(CO)n1cc2cc(-c3sc(C(=O)N4CCCNCC4)cc3-c3ccc(C#N)c(F)c3)ccc2n1 ; CC(C)(CO)n1nnc2cc(-c3sc(C(=O)N4CCCNCC4)cc3-c3ccc(C#N)c(F)c3)c(F)c(F)c21 ; CN(C)C1CCCN(C(=O)c2cc(-c3ccc(C#N)c(F)c3)c(-c3cc4nnn(CC(C)(C)O)c4c(F)c3F)s2)C1]

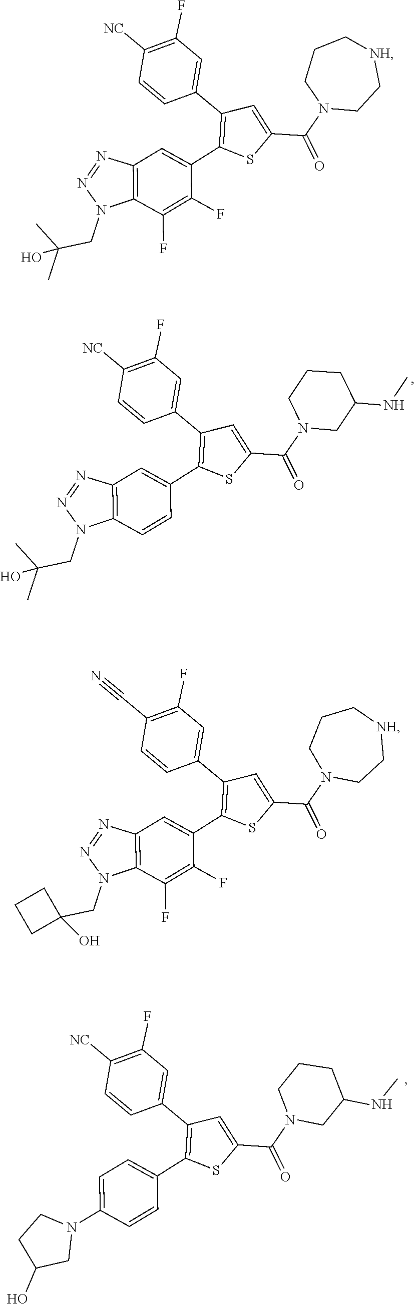 [SMILES: CC(C)(O)Cn1nnc2cc(-c3sc(C(=O)N4CCCNCC4)cc3-c3ccc(C#N)c(F)c3)c(F)c(F)c21 ; CNC1CCCN(C(=O)c2cc(-c3ccc(C#N)c(F)c3)c(-c3ccc4c(c3)nnn4CC(C)(C)O)s2)C1 ; N#Cc1ccc(-c2cc(C(=O)N3CCCNCC3)sc2-c2cc3nnn(CC4(O)CCC4)c3c(F)c2F)cc1F ; CNC1CCCN(C(=O)c2cc(-c3ccc(C#N)c(F)c3)c(-c3ccc(N4CCC(O)C4)cc3)s2)C1]

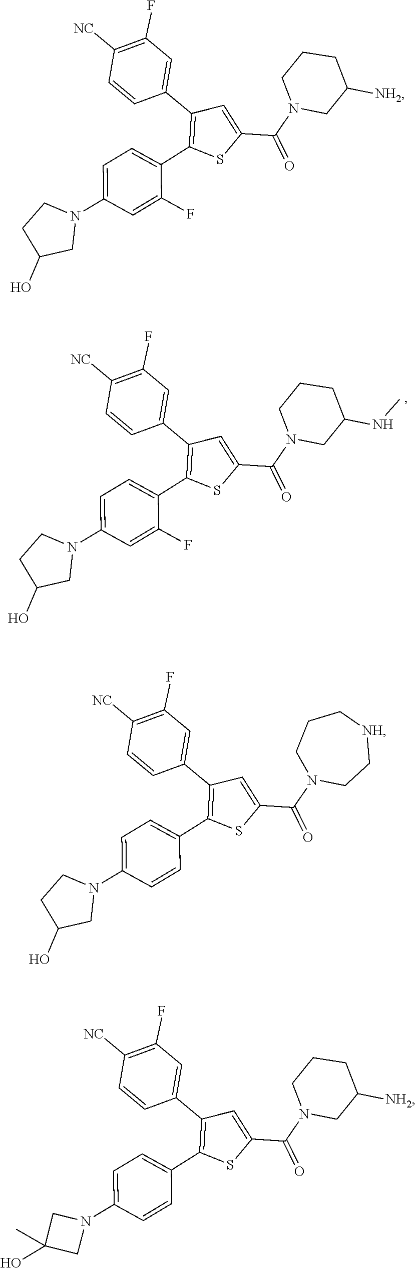

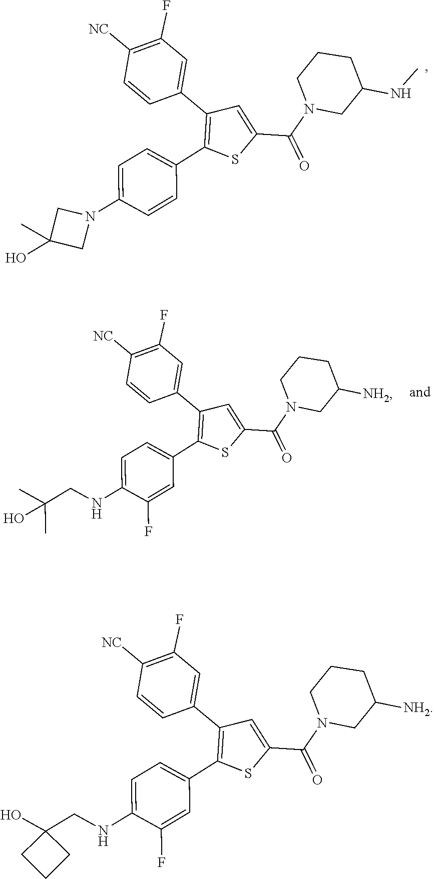

39. The compound or the pharmaceutically acceptable salt thereof according to claim 38, selected from,

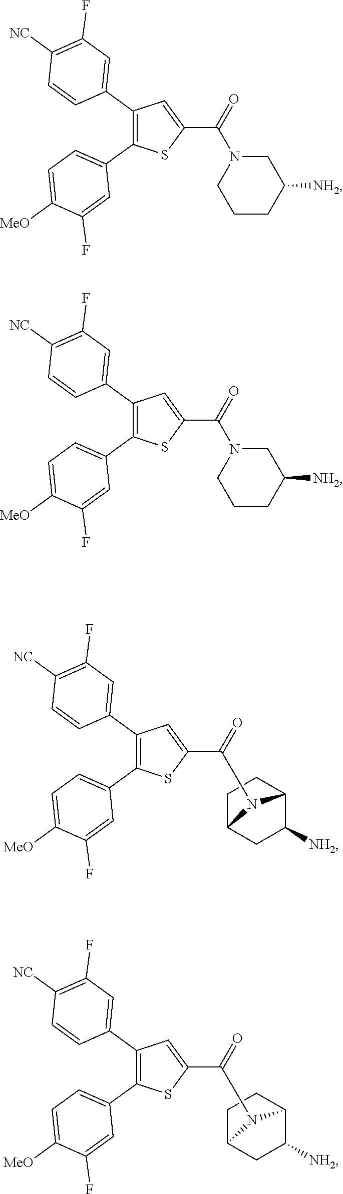 [SMILES: COc1ccc(-c2sc(C(=O)N3CCC[C@@H](N)C3)cc2-c2ccc(C#N)c(F)c2)cc1F ; COc1ccc(-c2sc(C(=O)N3CCC[C@H](N)C3)cc2-c2ccc(C#N)c(F)c2)cc1F ; COc1ccc(-c2sc(C(=O)N3[C@H]4CC[C@@H]3[C@@H](N)C4)cc2-c2ccc(C#N)c(F)c2)cc1F ; COc1ccc(-c2sc(C(=O)N3[C@@H]4CC[C@H]3[C@H](N)C4)cc2-c2ccc(C#N)c(F)c2)cc1F]

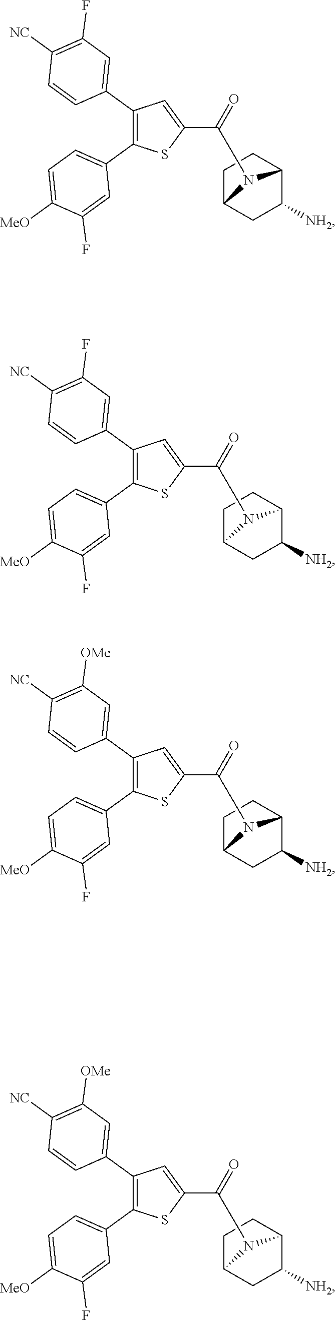 [SMILES: COc1ccc(-c2sc(C(=O)N3[C@H]4CC[C@@H]3[C@H](N)C4)cc2-c2ccc(C#N)c(F)c2)cc1F ; COc1ccc(-c2sc(C(=O)N3[C@@H]4CC[C@H]3[C@@H](N)C4)cc2-c2ccc(C#N)c(F)c2)cc1F ; COc1ccc(-c2sc(C(=O)N3[C@H]4CC[C@@H]3[C@@H](N)C4)cc2-c2ccc(C#N)c(OC)c2)cc1F ; COc1ccc(-c2sc(C(=O)N3[C@@H]4CC[C@H]3[C@H](N)C4)cc2-c2ccc(C#N)c(OC)c2)cc1F]

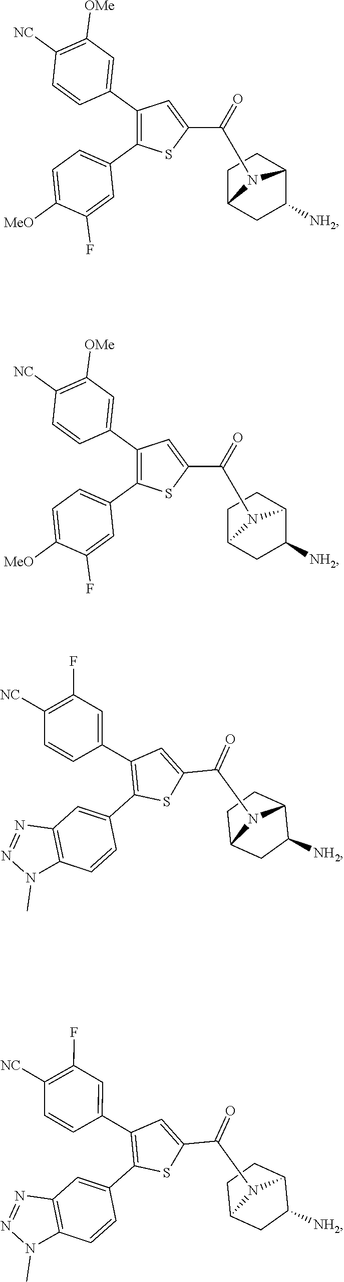 [SMILES: COc1ccc(-c2sc(C(=O)N3[C@H]4CC[C@@H]3[C@H](N)C4)cc2-c2ccc(C#N)c(OC)c2)cc1F ; COc1ccc(-c2sc(C(=O)N3[C@@H]4CC[C@H]3[C@@H](N)C4)cc2-c2ccc(C#N)c(OC)c2)cc1F ; Cn1nnc2cc(-c3sc(C(=O)N4[C@H]5CC[C@@H]4[C@@H](N)C5)cc3-c3ccc(C#N)c(F)c3)ccc21 ; Cn1nnc2cc(-c3sc(C(=O)N4[C@@H]5CC[C@H]4[C@H](N)C5)cc3-c3ccc(C#N)c(F)c3)ccc21]

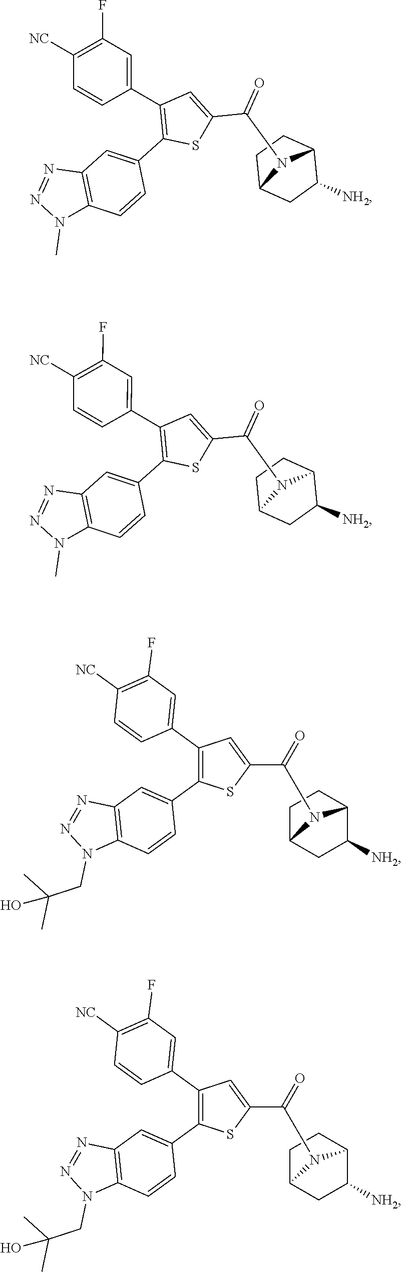 [SMILES: Cn1nnc2cc(-c3sc(C(=O)N4[C@H]5CC[C@@H]4[C@H](N)C5)cc3-c3ccc(C#N)c(F)c3)ccc21 ; Cn1nnc2cc(-c3sc(C(=O)N4[C@@H]5CC[C@H]4[C@@H](N)C5)cc3-c3ccc(C#N)c(F)c3)ccc21 ; CC(C)(O)Cn1nnc2cc(-c3sc(C(=O)N4[C@H]5CC[C@@H]4[C@@H](N)C5)cc3-c3ccc(C#N)c(F)c3)ccc21 ; CC(C)(O)Cn1nnc2cc(-c3sc(C(=O)N4[C@@H]5CC[C@H]4[C@H](N)C5)cc3-c3ccc(C#N)c(F)c3)ccc21]

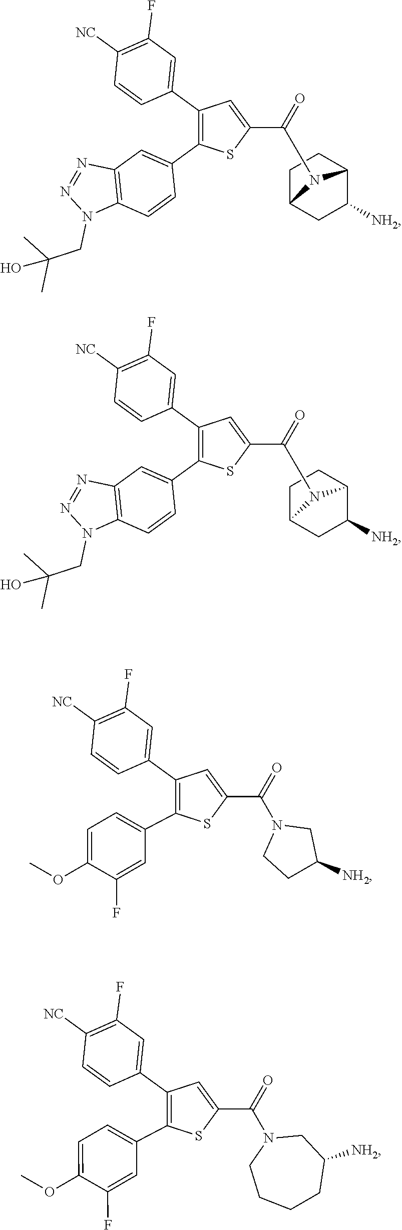 [SMILES: CC(C)(O)Cn1nnc2cc(-c3sc(C(=O)N4[C@H]5CC[C@@H]4[C@H](N)C5)cc3-c3ccc(C#N)c(F)c3)ccc21 ; CC(C)(O)Cn1nnc2cc(-c3sc(C(=O)N4[C@@H]5CC[C@H]4[C@@H](N)C5)cc3-c3ccc(C#N)c(F)c3)ccc21 ; COc1ccc(-c2sc(C(=O)N3CC[C@H](N)C3)cc2-c2ccc(C#N)c(F)c2)cc1F ; COc1ccc(-c2sc(C(=O)N3CCCC[C@@H](N)C3)cc2-c2ccc(C#N)c(F)c2)cc1F]

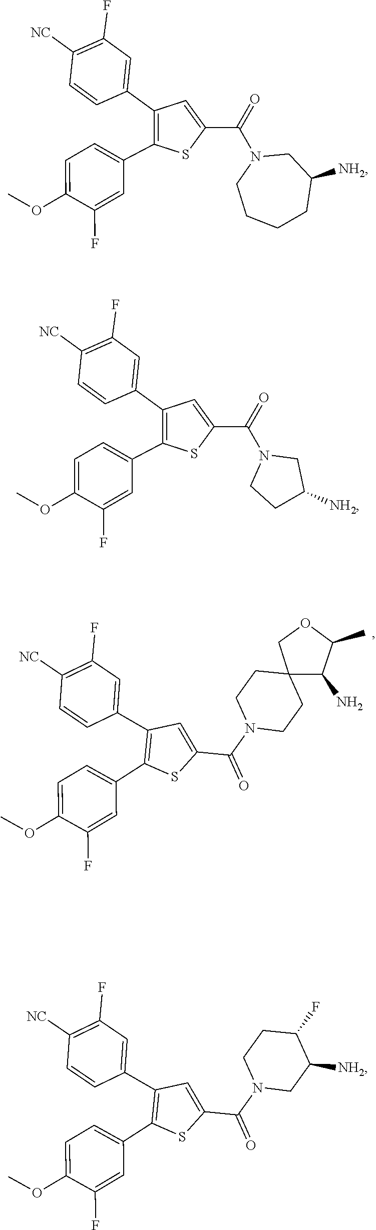

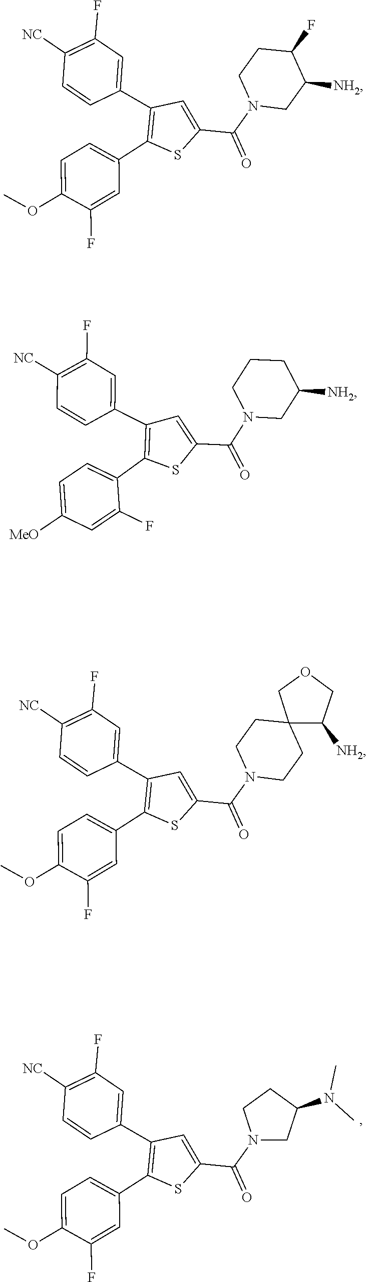

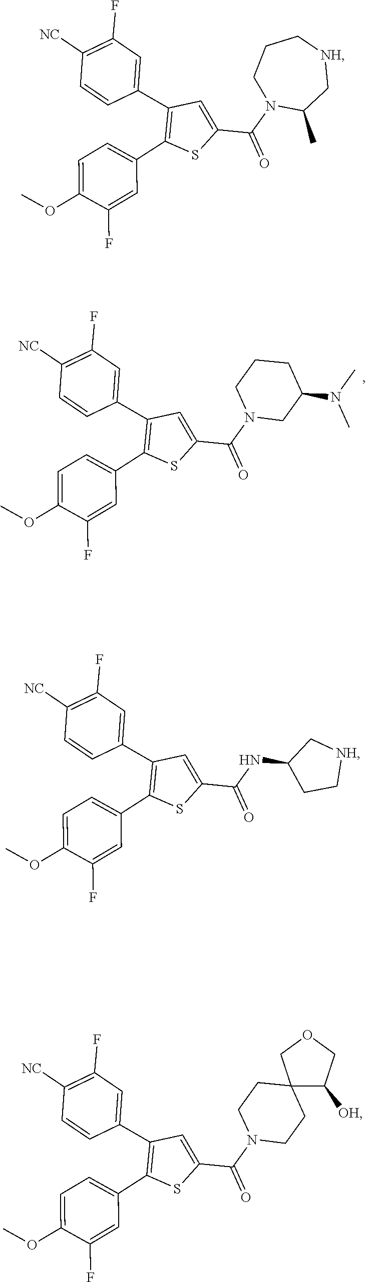

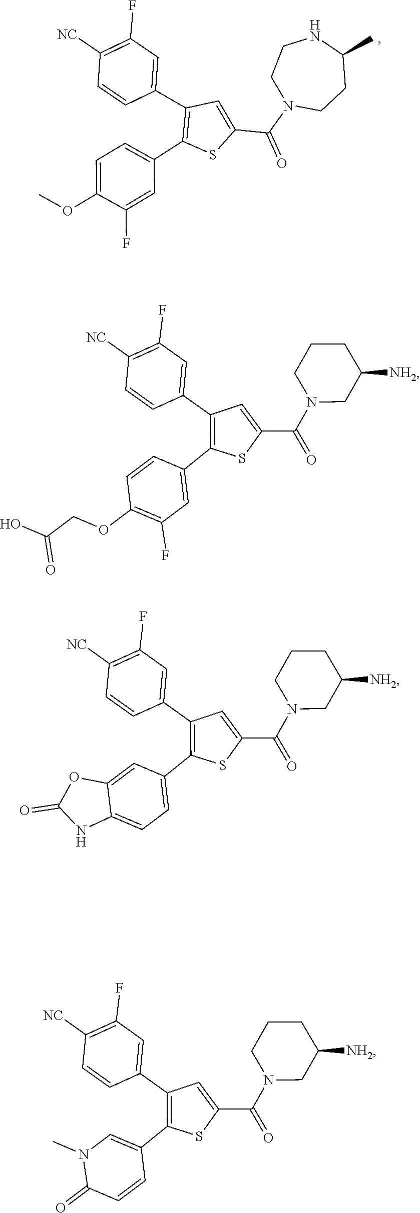

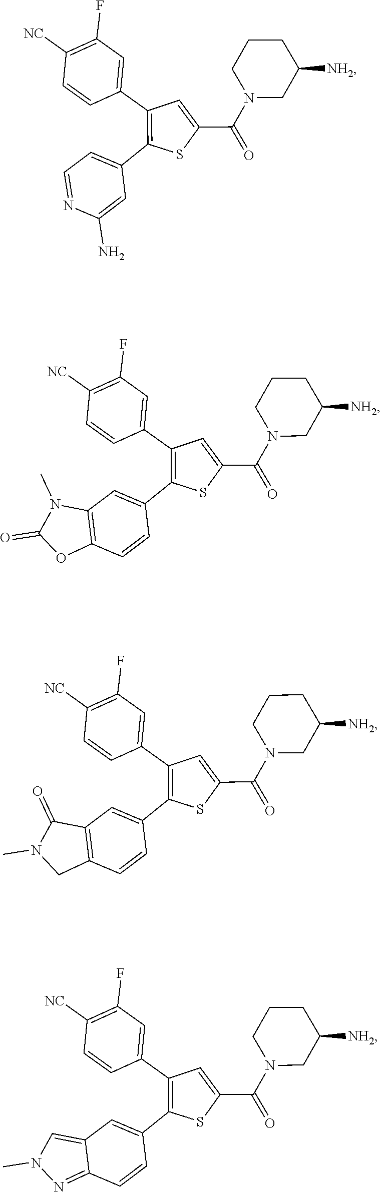 [SMILES: N#Cc1ccc(-c2cc(C(=O)N3CCC[C@@H](N)C3)sc2-c2ccnc(N)c2)cc1F ; Cn1c(=O)oc2ccc(-c3sc(C(=O)N4CCC[C@@H](N)C4)cc3-c3ccc(C#N)c(F)c3)cc21 ; CN1Cc2ccc(-c3sc(C(=O)N4CCC[C@@H](N)C4)cc3-c3ccc(C#N)c(F)c3)cc2C1=O ; Cn1cc2cc(-c3sc(C(=O)N4CCC[C@@H](N)C4)cc3-c3ccc(C#N)c(F)c3)ccc2n1]

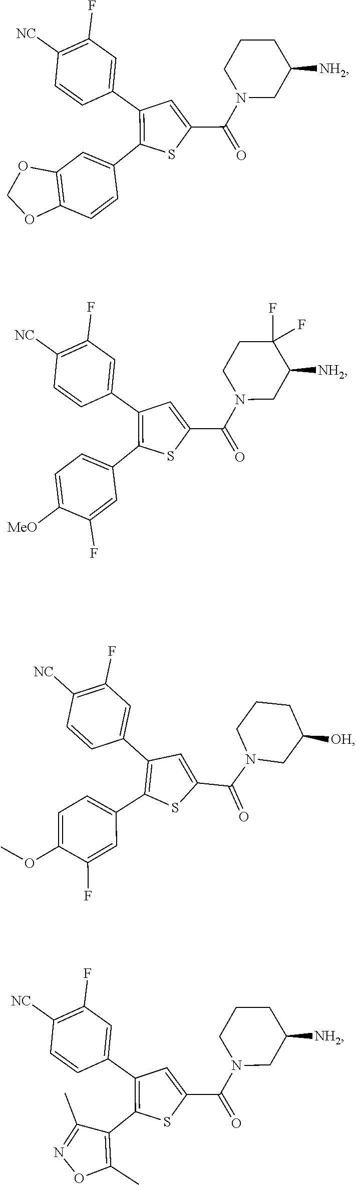 [SMILES: N#Cc1ccc(-c2cc(C(=O)N3CCC[C@@H](N)C3)sc2-c2ccc3c(c2)OCO3)cc1F ; COc1ccc(-c2sc(C(=O)N3CCC(F)(F)[C@@H](N)C3)cc2-c2ccc(C#N)c(F)c2)cc1F ; COc1ccc(-c2sc(C(=O)N3CCC[C@@H](O)C3)cc2-c2ccc(C#N)c(F)c2)cc1F ; Cc1noc(C)c1-c1sc(C(=O)N2CCC[C@@H](N)C2)cc1-c1ccc(C#N)c(F)c1]

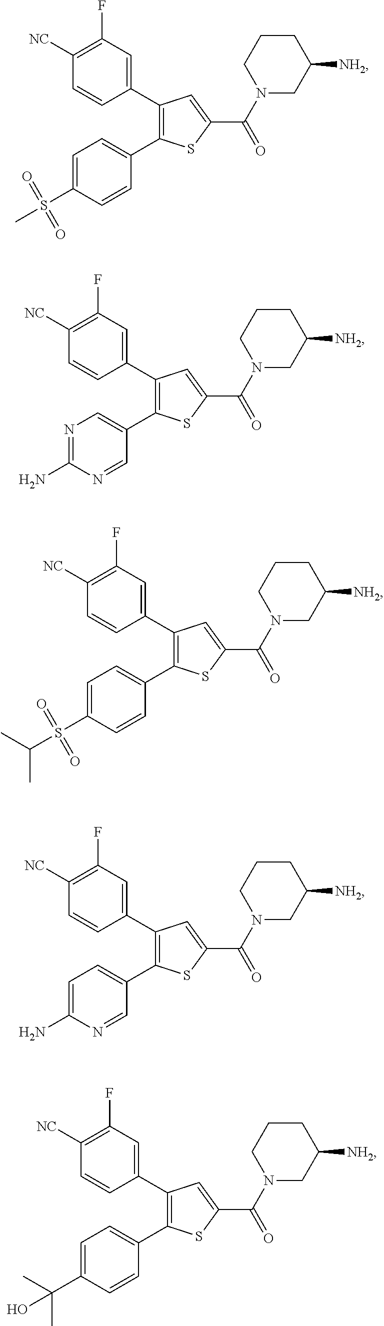 [SMILES: CS(=O)(=O)c1ccc(-c2sc(C(=O)N3CCC[C@@H](N)C3)cc2-c2ccc(C#N)c(F)c2)cc1 ; N#Cc1ccc(-c2cc(C(=O)N3CCC[C@@H](N)C3)sc2-c2cnc(N)nc2)cc1F ; CC(C)S(=O)(=O)c1ccc(-c2sc(C(=O)N3CCC[C@@H](N)C3)cc2-c2ccc(C#N)c(F)c2)cc1 ; N#Cc1ccc(-c2cc(C(=O)N3CCC[C@@H](N)C3)sc2-c2ccc(N)nc2)cc1F ; CC(C)(O)c1ccc(-c2sc(C(=O)N3CCC[C@@H](N)C3)cc2-c2ccc(C#N)c(F)c2)cc1]

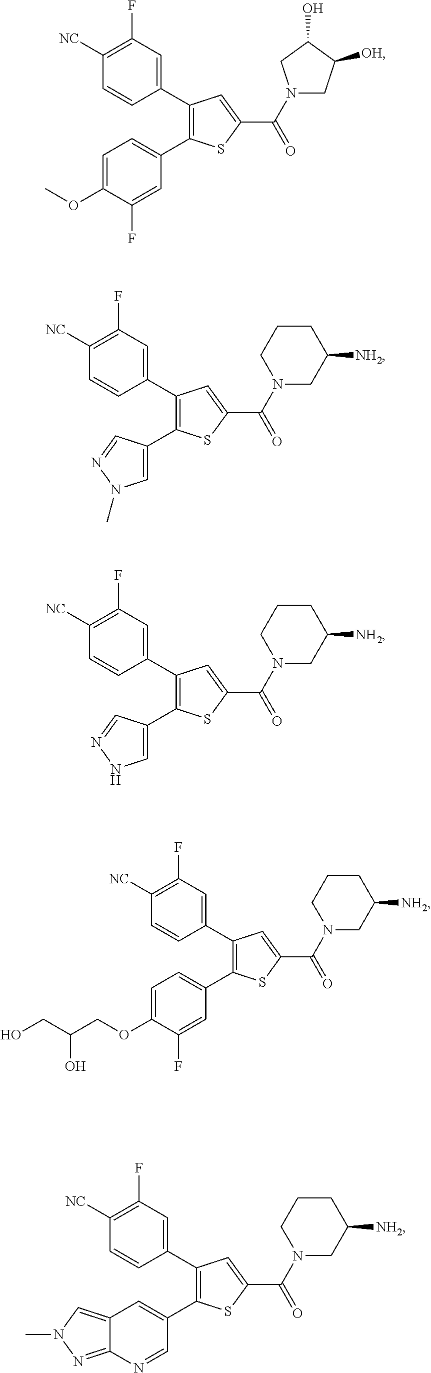 [SMILES: COc1ccc(-c2sc(C(=O)N3C[C@H](O)[C@@H](O)C3)cc2-c2ccc(C#N)c(F)c2)cc1F ; Cn1cc(-c2sc(C(=O)N3CCC[C@@H](N)C3)cc2-c2ccc(C#N)c(F)c2)cn1 ; N#Cc1ccc(-c2cc(C(=O)N3CCC[C@@H](N)C3)sc2-c2cn[nH]c2)cc1F ; N#Cc1ccc(-c2cc(C(=O)N3CCC[C@@H](N)C3)sc2-c2ccc(OCC(O)CO)c(F)c2)cc1F ; Cn1cc2cc(-c3sc(C(=O)N4CCC[C@@H](N)C4)cc3-c3ccc(C#N)c(F)c3)cnc2n1]

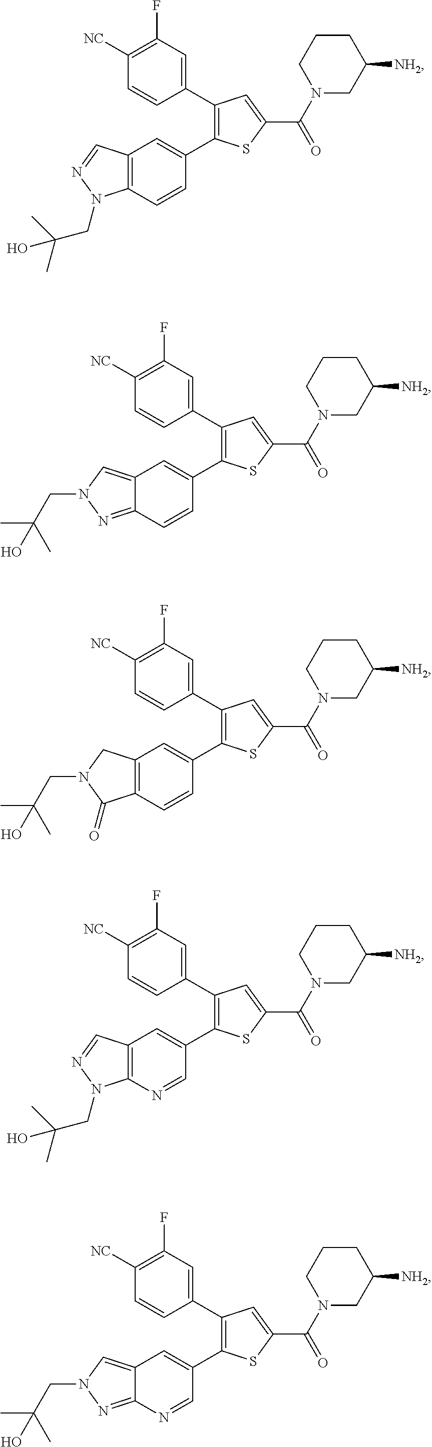 [SMILES: CC(C)(O)Cn1ncc2cc(-c3sc(C(=O)N4CCC[C@@H](N)C4)cc3-c3ccc(C#N)c(F)c3)ccc21 ; CC(C)(O)Cn1cc2cc(-c3sc(C(=O)N4CCC[C@@H](N)C4)cc3-c3ccc(C#N)c(F)c3)ccc2n1 ; CC(C)(O)CN1Cc2cc(-c3sc(C(=O)N4CCC[C@@H](N)C4)cc3-c3ccc(C#N)c(F)c3)ccc2C1=O ; CC(C)(O)Cn1ncc2cc(-c3sc(C(=O)N4CCC[C@@H](N)C4)cc3-c3ccc(C#N)c(F)c3)cnc21 ; CC(C)(O)Cn1cc2cc(-c3sc(C(=O)N4CCC[C@@H](N)C4)cc3-c3ccc(C#N)c(F)c3)cnc2n1]

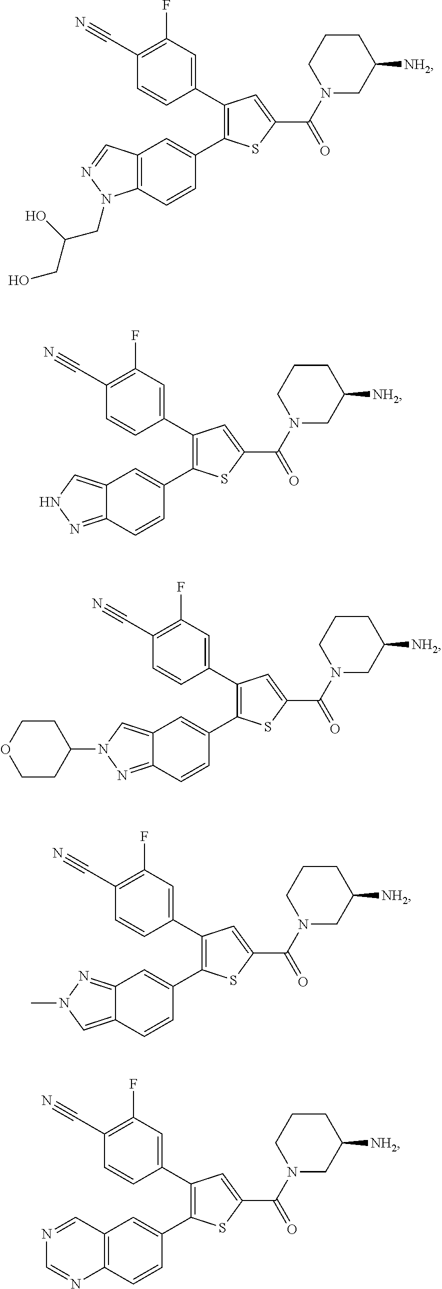 [SMILES: N#Cc1ccc(-c2cc(C(=O)N3CCC[C@@H](N)C3)sc2-c2ccc3c(cnn3CC(O)CO)c2)cc1F ; N#Cc1ccc(-c2cc(C(=O)N3CCC[C@@H](N)C3)sc2-c2ccc3n[nH]cc3c2)cc1F ; N#Cc1ccc(-c2cc(C(=O)N3CCC[C@@H](N)C3)sc2-c2ccc3nn(C4CCOCC4)cc3c2)cc1F ; Cn1cc2ccc(-c3sc(C(=O)N4CCC[C@@H](N)C4)cc3-c3ccc(C#N)c(F)c3)cc2n1 ; N#Cc1ccc(-c2cc(C(=O)N3CCC[C@@H](N)C3)sc2-c2ccc3ncncc3c2)cc1F]

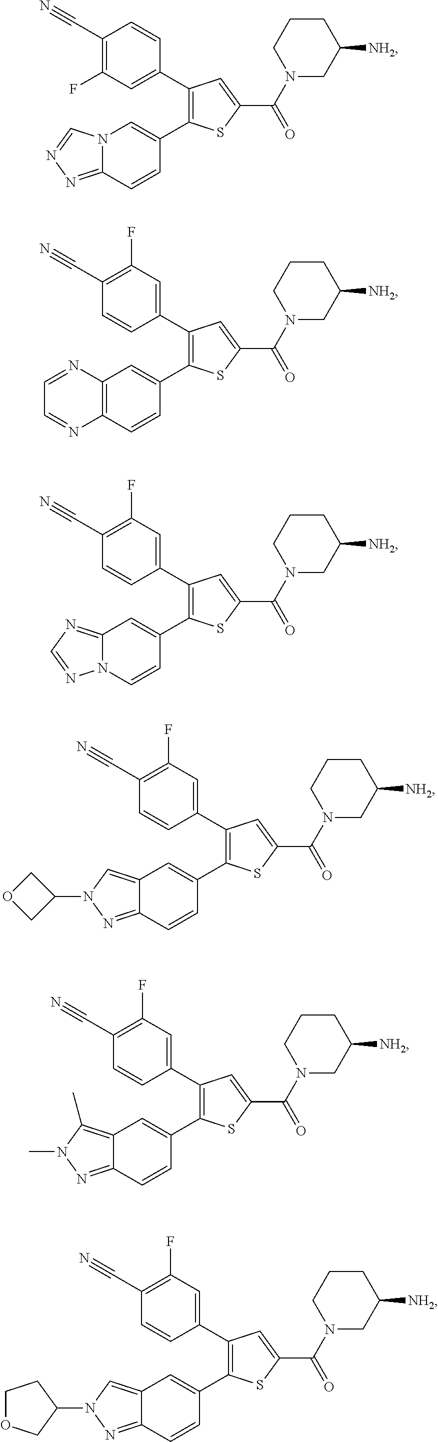

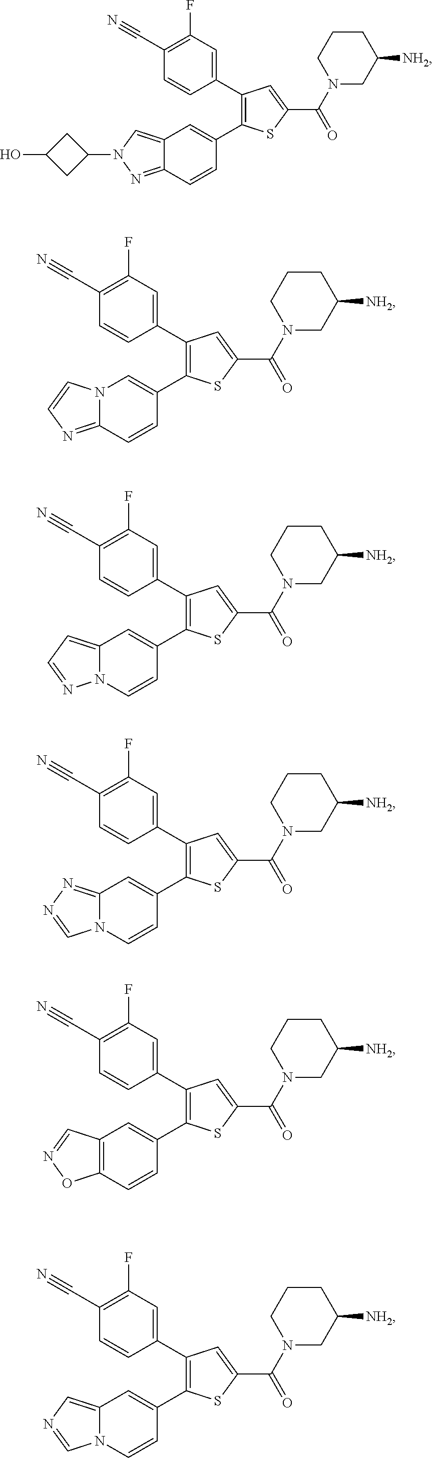

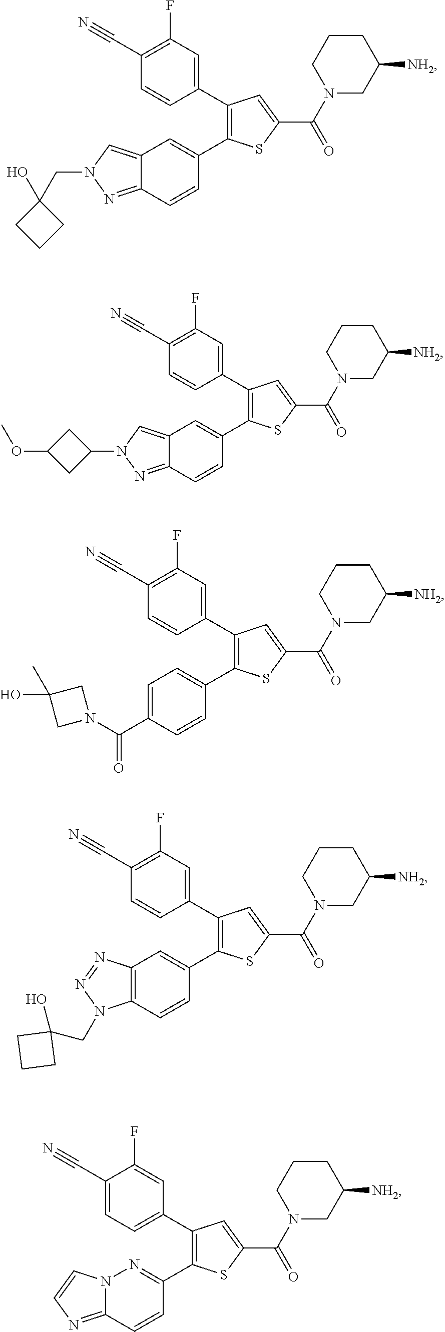 [SMILES: N#Cc1ccc(-c2cc(C(=O)N3CCC[C@@H](N)C3)sc2-c2ccc3nn(CC4(O)CCC4)cc3c2)cc1F ; COC1CC(n2cc3cc(-c4sc(C(=O)N5CCC[C@@H](N)C5)cc4-c4ccc(C#N)c(F)c4)ccc3n2)C1 ; CC1(O)CN(C(=O)c2ccc(-c3sc(C(=O)N4CCC[C@@H](N)C4)cc3-c3ccc(C#N)c(F)c3)cc2)C1 ; N#Cc1ccc(-c2cc(C(=O)N3CCC[C@@H](N)C3)sc2-c2ccc3c(c2)nnn3CC2(O)CCC2)cc1F ; N#Cc1ccc(-c2cc(C(=O)N3CCC[C@@H](N)C3)sc2-c2ccc3nccn3n2)cc1F]

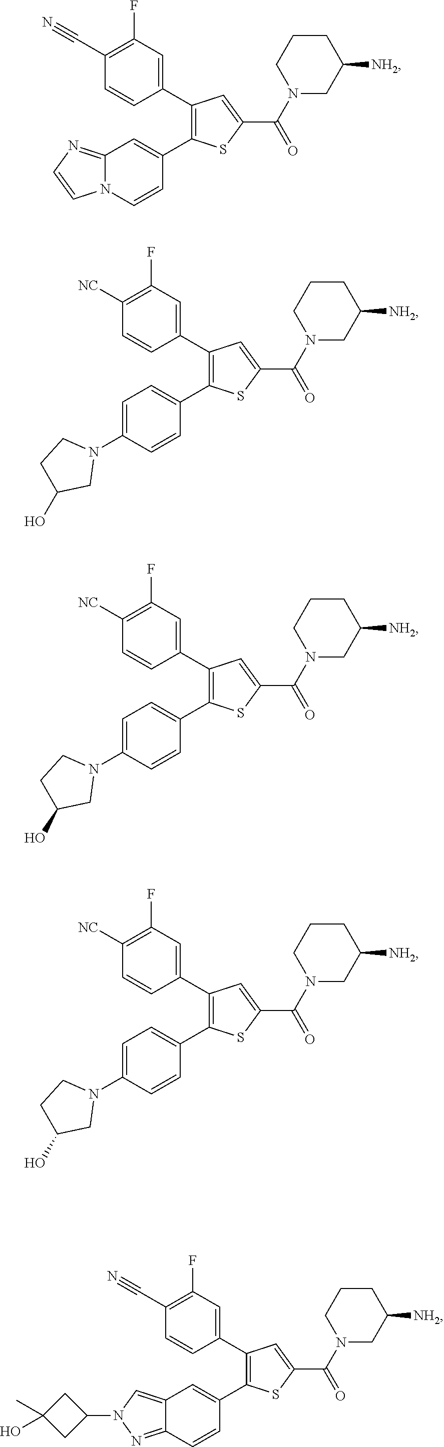 [SMILES: N#Cc1ccc(-c2cc(C(=O)N3CCC[C@@H](N)C3)sc2-c2ccn3ccnc3c2)cc1F ; N#Cc1ccc(-c2cc(C(=O)N3CCC[C@@H](N)C3)sc2-c2ccc(N3CCC(O)C3)cc2)cc1F ; N#Cc1ccc(-c2cc(C(=O)N3CCC[C@@H](N)C3)sc2-c2ccc(N3CC[C@H](O)C3)cc2)cc1F ; N#Cc1ccc(-c2cc(C(=O)N3CCC[C@@H](N)C3)sc2-c2ccc(N3CC[C@@H](O)C3)cc2)cc1F ; CC1(O)CC(n2cc3cc(-c4sc(C(=O)N5CCC[C@@H](N)C5)cc4-c4ccc(C#N)c(F)c4)ccc3n2)C1]

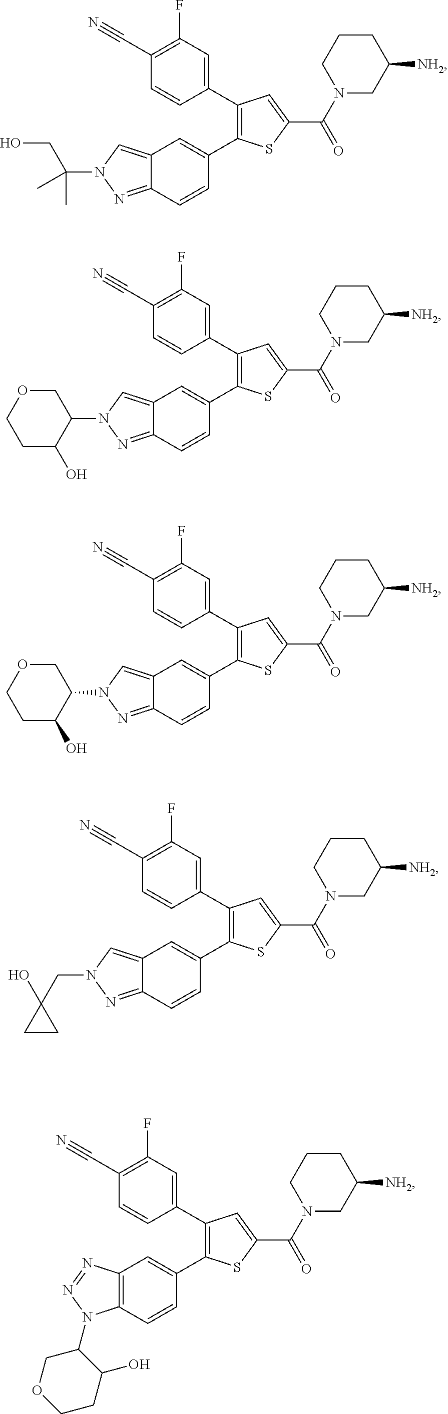 [SMILES: CC(C)(CO)n1cc2cc(-c3sc(C(=O)N4CCC[C@@H](N)C4)cc3-c3ccc(C#N)c(F)c3)ccc2n1 ; N#Cc1ccc(-c2cc(C(=O)N3CCC[C@@H](N)C3)sc2-c2ccc3nn(C4COCCC4O)cc3c2)cc1F ; N#Cc1ccc(-c2cc(C(=O)N3CCC[C@@H](N)C3)sc2-c2ccc3nn([C@H]4COCC[C@@H]4O)cc3c2)cc1F ; N#Cc1ccc(-c2cc(C(=O)N3CCC[C@@H](N)C3)sc2-c2ccc3nn(CC4(O)CC4)cc3c2)cc1F ; N#Cc1ccc(-c2cc(C(=O)N3CCC[C@@H](N)C3)sc2-c2ccc3c(c2)nnn3C2COCCC2O)cc1F]

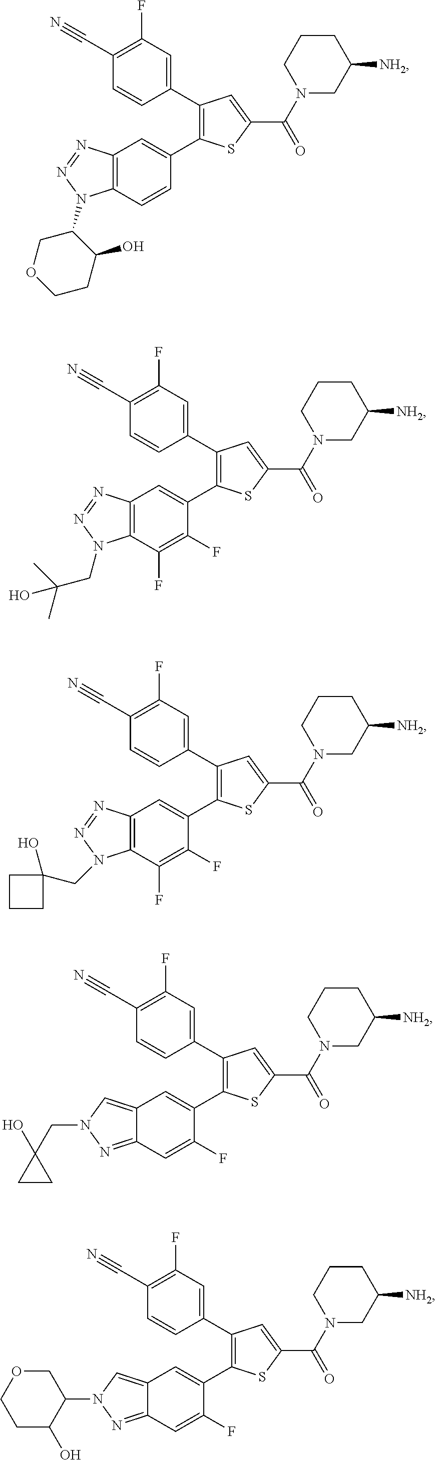 [SMILES: N#Cc1ccc(-c2cc(C(=O)N3CCC[C@@H](N)C3)sc2-c2ccc3c(c2)nnn3[C@H]2COCC[C@@H]2O)cc1F ; CC(C)(O)Cn1nnc2cc(-c3sc(C(=O)N4CCC[C@@H](N)C4)cc3-c3ccc(C#N)c(F)c3)c(F)c(F)c21 ; N#Cc1ccc(-c2cc(C(=O)N3CCC[C@@H](N)C3)sc2-c2cc3nnn(CC4(O)CCC4)c3c(F)c2F)cc1F ; N#Cc1ccc(-c2cc(C(=O)N3CCC[C@@H](N)C3)sc2-c2cc3cn(CC4(O)CC4)nc3cc2F)cc1F ; N#Cc1ccc(-c2cc(C(=O)N3CCC[C@@H](N)C3)sc2-c2cc3cn(C4COCCC4O)nc3cc2F)cc1F]

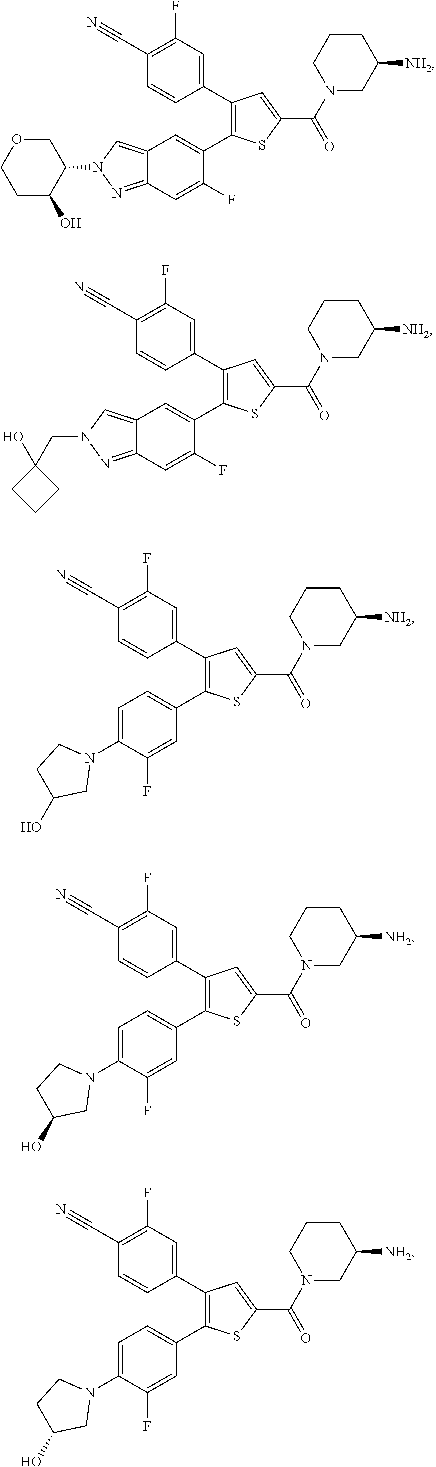 [SMILES: N#Cc1ccc(-c2cc(C(=O)N3CCC[C@@H](N)C3)sc2-c2cc3cn([C@H]4COCC[C@@H]4O)nc3cc2F)cc1F ; N#Cc1ccc(-c2cc(C(=O)N3CCC[C@@H](N)C3)sc2-c2cc3cn(CC4(O)CCC4)nc3cc2F)cc1F ; N#Cc1ccc(-c2cc(C(=O)N3CCC[C@@H](N)C3)sc2-c2ccc(N3CCC(O)C3)c(F)c2)cc1F ; N#Cc1ccc(-c2cc(C(=O)N3CCC[C@@H](N)C3)sc2-c2ccc(N3CC[C@H](O)C3)c(F)c2)cc1F ; N#Cc1ccc(-c2cc(C(=O)N3CCC[C@@H](N)C3)sc2-c2ccc(N3CC[C@@H](O)C3)c(F)c2)cc1F]

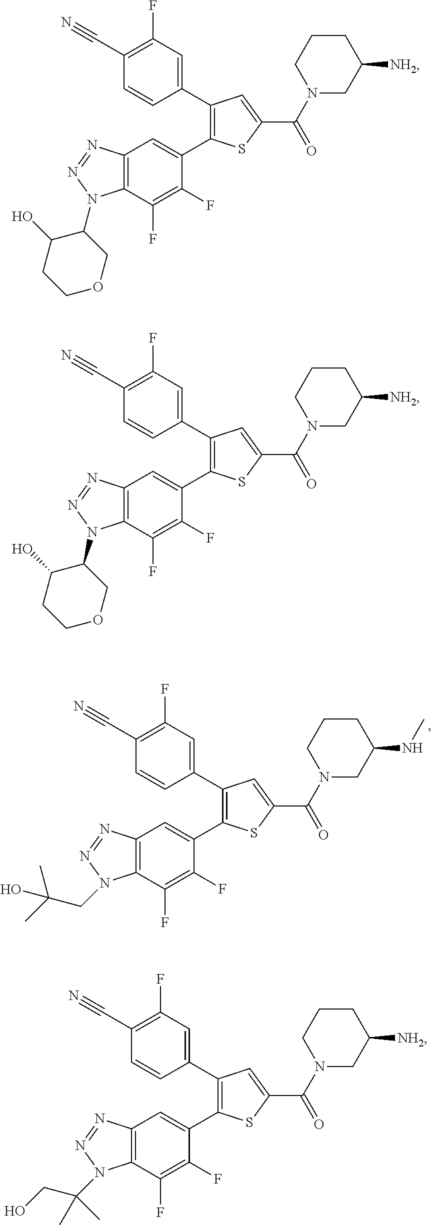 [SMILES: N#Cc1ccc(-c2cc(C(=O)N3CCC[C@@H](N)C3)sc2-c2cc3nnn(C4COCCC4O)c3c(F)c2F)cc1F ; N#Cc1ccc(-c2cc(C(=O)N3CCC[C@@H](N)C3)sc2-c2cc3nnn([C@H]4COCC[C@@H]4O)c3c(F)c2F)cc1F ; CN[C@@H]1CCCN(C(=O)c2cc(-c3ccc(C#N)c(F)c3)c(-c3cc4nnn(CC(C)(C)O)c4c(F)c3F)s2)C1 ; CC(C)(CO)n1nnc2cc(-c3sc(C(=O)N4CCC[C@@H](N)C4)cc3-c3ccc(C#N)c(F)c3)c(F)c(F)c21]

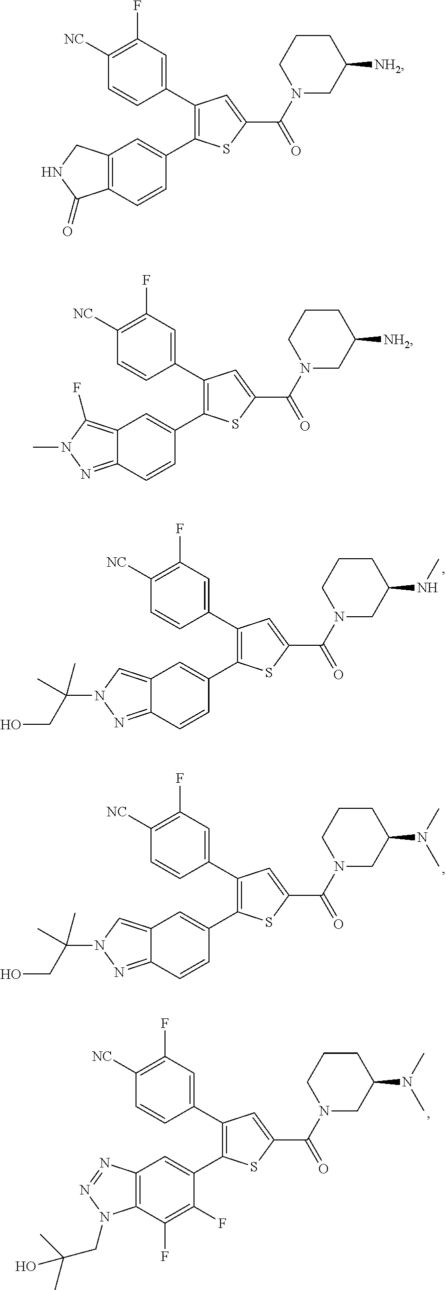 [SMILES: N#Cc1ccc(-c2cc(C(=O)N3CCC[C@@H](N)C3)sc2-c2ccc3c(c2)CNC3=O)cc1F ; Cn1nc2ccc(-c3sc(C(=O)N4CCC[C@@H](N)C4)cc3-c3ccc(C#N)c(F)c3)cc2c1F ; CN[C@@H]1CCCN(C(=O)c2cc(-c3ccc(C#N)c(F)c3)c(-c3ccc4nn(C(C)(C)CO)cc4c3)s2)C1 ; CN(C)[C@@H]1CCCN(C(=O)c2cc(-c3ccc(C#N)c(F)c3)c(-c3ccc4nn(C(C)(C)CO)cc4c3)s2)C1 ; CN(C)[C@@H]1CCCN(C(=O)c2cc(-c3ccc(C#N)c(F)c3)c(-c3cc4nnn(CC(C)(C)O)c4c(F)c3F)s2)C1]

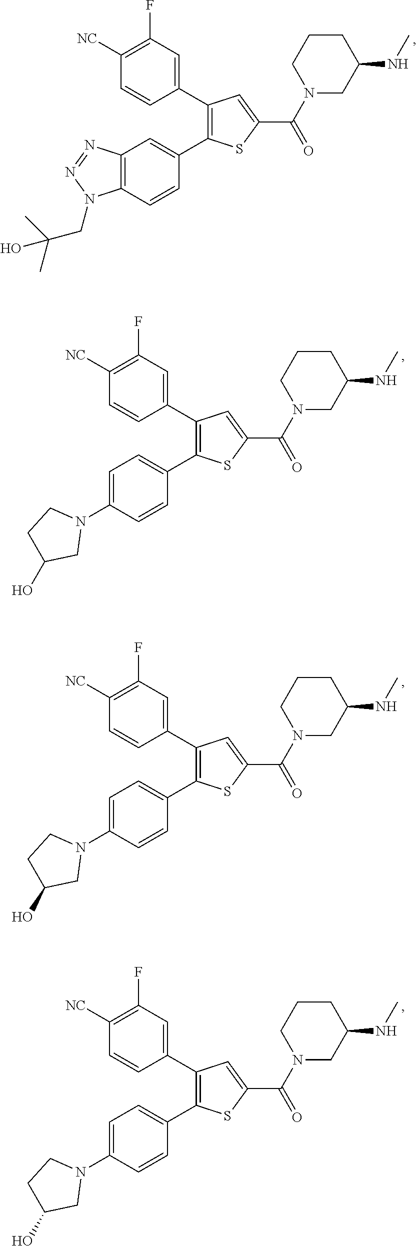 [SMILES: CN[C@@H]1CCCN(C(=O)c2cc(-c3ccc(C#N)c(F)c3)c(-c3ccc4c(c3)nnn4CC(C)(C)O)s2)C1 ; CN[C@@H]1CCCN(C(=O)c2cc(-c3ccc(C#N)c(F)c3)c(-c3ccc(N4CCC(O)C4)cc3)s2)C1 ; CN[C@@H]1CCCN(C(=O)c2cc(-c3ccc(C#N)c(F)c3)c(-c3ccc(N4CC[C@H](O)C4)cc3)s2)C1 ; CN[C@@H]1CCCN(C(=O)c2cc(-c3ccc(C#N)c(F)c3)c(-c3ccc(N4CC[C@@H](O)C4)cc3)s2)C1]

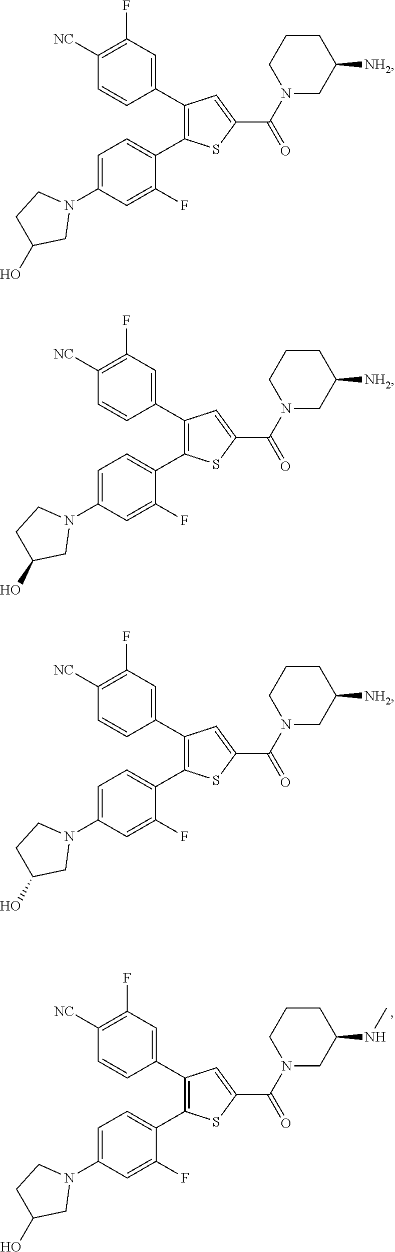 [SMILES: N#Cc1ccc(-c2cc(C(=O)N3CCC[C@@H](N)C3)sc2-c2ccc(N3CCC(O)C3)cc2F)cc1F ; N#Cc1ccc(-c2cc(C(=O)N3CCC[C@@H](N)C3)sc2-c2ccc(N3CC[C@H](O)C3)cc2F)cc1F ; N#Cc1ccc(-c2cc(C(=O)N3CCC[C@@H](N)C3)sc2-c2ccc(N3CC[C@@H](O)C3)cc2F)cc1F ; CN[C@@H]1CCCN(C(=O)c2cc(-c3ccc(C#N)c(F)c3)c(-c3ccc(N4CCC(O)C4)cc3F)s2)C1]

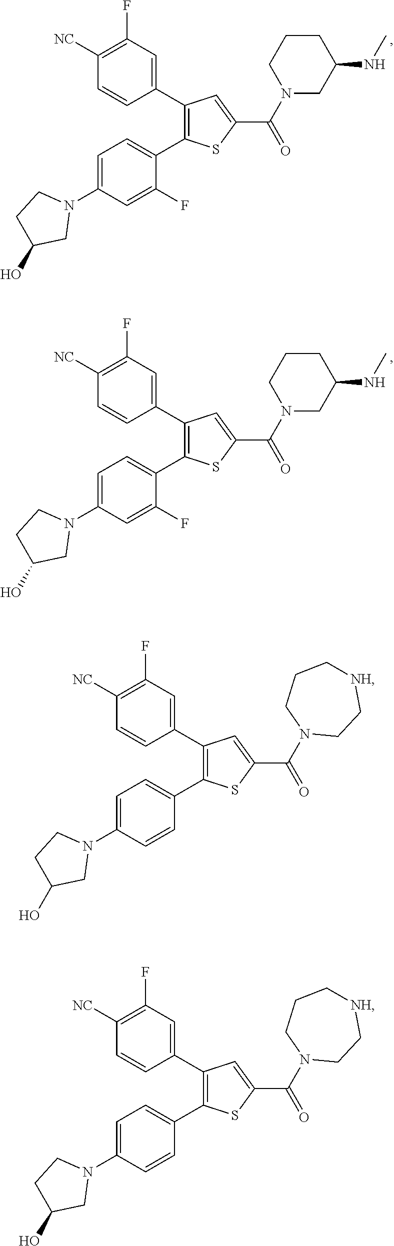 [SMILES: CN[C@@H]1CCCN(C(=O)c2cc(-c3ccc(C#N)c(F)c3)c(-c3ccc(N4CC[C@H](O)C4)cc3F)s2)C1 ; CN[C@@H]1CCCN(C(=O)c2cc(-c3ccc(C#N)c(F)c3)c(-c3ccc(N4CC[C@@H](O)C4)cc3F)s2)C1 ; N#Cc1ccc(-c2cc(C(=O)N3CCCNCC3)sc2-c2ccc(N3CCC(O)C3)cc2)cc1F ; N#Cc1ccc(-c2cc(C(=O)N3CCCNCC3)sc2-c2ccc(N3CC[C@H](O)C3)cc2)cc1F]

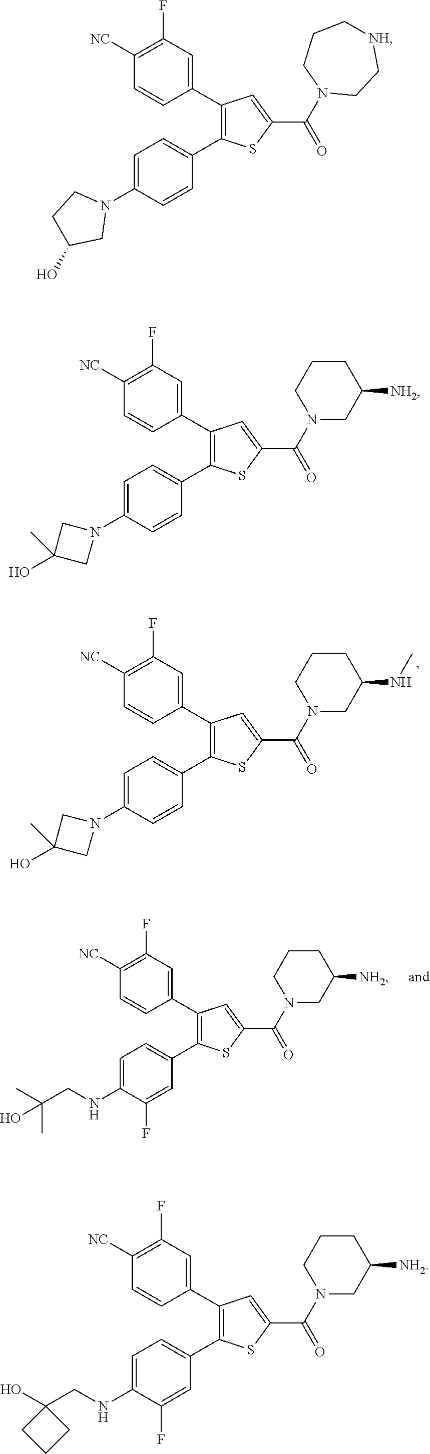

40. (canceled)

41. A method for treating diseases related to LSD1 in a subject in need thereof, comprising administering the compound or the pharmaceutically acceptable salt thereof according to claim 1 to the subject.
